(19) United States
(12) Patent Application Publication
(10) Pub. No.: US 2023/0061432 A1
(43) Pub. Date: Mar. 2, 2023

(54) NEW TECHNOLOGY TO CONJUGATE THE TACCALONOLIDE MICROTUBULE STABILIZERS WITH LINKERS/PAYLOADS

(71) Applicants: Lin Du, Norman, OK (US); April Risinger, San Antonio, TX (US); Samantha Yee, San Antonio, TX (US)

(72) Inventors: Lin Du, Norman, OK (US); April Risinger, San Antonio, TX (US); Samantha Yee, San Antonio, TX (US)

(21) Appl. No.: 17/437,379

(22) PCT Filed: Mar. 6, 2020
(86) PCT No.: PCT/US2020/021522

§ 371 Date: Sep. 8, 2021

Related U.S. Application Data
(60) Provisional application No. 62/815,833, filed on Mar. 8, 2019.

(51) Int. Cl. C07J 53/00; C07J 71/00; A61K 31/585; A61P 35/00

(52) U.S. Cl. CPC C07J 53/002; C07J 71/0005; A61K 31/585; A61P 35/00

(57) ABSTRACT

The present disclosure is concerned with taccalonolide analogs and conjugated taccalonolide analogs useful as cellular probes and in the treatment of, for example, hyperproliferative disorders such as cardiovascular diseases and cancer. This abstract is intended as a scanning tool for purposes of searching in the particular art and is not intended to be limiting of the present invention.

### CROSS-REFERENCE TO RELATED APPLICATIONS

This application claims the benefit of U.S. Application No. 62/815,833, filed on Mar. 8, 2019, the contents of which are hereby incorporated by reference in their entirety.

### STATEMENT REGARDING FEDERALLY SPONSORED RESEARCH

This invention was made with government support under grant number 1R01 CA219948 awarded by the National Institutes of Health. The government has certain rights in the invention.

### REFERENCE TO SEQUENCE LISTING

The Sequence Listing submitted Mar. 6, 2020 as a text file named “21105_0066P1_ST25.txt,” created on Feb. 29, 2020, and having a size of 8,049 bytes is hereby incorporated by reference pursuant to 37 C.F.R. § 1.52(e)(5).

### BACKGROUND

Microtubules are cellular structures important for normal cellular metabolism, cellular transport, and cell division. Interrupting microtubule dependent processes causes cellular effects including inhibition of proliferation and cellular trafficking which, in turn, lead to initiation of cell death pathways. Microtubule disrupting agents such as microtubule stabilizers are one of the most important classes of anticancer therapeutics used in the clinic today. Additionally, microtubule stabilizers are used in other human diseases of hyperproliferation including, but not limited to, cardiovascular disease, where they are used to coat stents. The taxoid microtubule stabilizer paclitaxel (Taxol™) has been widely used in the treatment of solid tumors including breast, ovarian, and lung cancers, for over a decade as a single agent and in combination with targeted therapies. In spite of their clinical utility, the shortcomings of paclitaxel and the second generation semi-synthetic taxoid, docetaxel (Taxotere™), include innate and acquired drug resistance and dose-limiting toxicities (Fojo and Menefee, 2007). Two new microtubule stabilizers have recently been approved for clinical use—the epothilone ixabepilone (Ixempra) and the taxoid cabazitaxel (Jevtana), which circumvent some, but not all, of the shortcomings of first and second generation microtubule stabilizers (Morris and Fornier, 2008; Galsky et al., 2010; Shen et al., 2011). These microtubule stabilizing drugs all bind to the interior lumen of the intact microtubule at the taxoid binding site, which causes a stabilization of microtubule protofilament interactions and thereby decreases the dynamic nature of microtubules (Nogales et al., 1995).

Two additional classes of microtubule stabilizers have been isolated from nature: laulimalides/peloruside A and the taccalonolides. Laulimalide and peloruside A have recently been shown to bind to the exterior of the microtubule at a site distinct from the taxoid binding site, but result in microtubule stabilization effects nearly identical to the toxoids (Bennett et al., 2010). The microtubule stabilizing properties of the taccalonolides A, E, B, and N, together with their ability to overcome multiple clinically relevant mechanisms of drug resistance (Risinger et al. 2008) prompted further interest in identifying new taccalonolides.

Intense efforts over the past three decades have identified a large variety of interesting chemical compounds from the roots and rhizomes of Tacca species, including 25 taccalonolides, denoted as taccalonolides A-Y (Chen et al. 1987; Chen et al. 1988; Shen et al. 1991; Shen et al. 1996; Chen et al. 1997; WO/2001/040256; Huang and Liu 2002; Muhlbauer et al. 2003; Yang et al. 2008). However, there have been limited biological studies on the taccalonolides. In 2003, microtubule stabilizing activities of taccalonolides A and E were reported (Tinley et al. 2003). Follow-up studies showed preliminary structure-activity relationships (SARs) for the antiproliferative activities of taccalonolides A, E, B, and N. The antiproliferative potencies of these four taccalonolides in HeLa cells were all in the mid-nanomolar range (190 nM to 644 nM) (Risinger et al. 2008) and further studies showed that the taccalonolides A, E, and N have in vivo antitumor activity (Peng et al. 2011). The discovery that the C22,23 double bond in the naturally occurring taccalonolides could be epoxidated semi-synthetically led to the identification that this epoxide facilitated the direct, covalent interaction of the taccalonolides with tubulin/microtubules (Li et al. 2011; Risinger et al. 2013; Peng et al. 2014). Taccalonolides bearing this C22,23 epoxide have potency in the low nanomolar range and it is proposed that the activity of non-epoxidated compounds previously reported is due to minor amounts of epoxidated species present. However, a full understanding of the structure-activity relationships of the taccalonolides remains to be elucidated. Given that the biological activity profiles of known taccalonolides vary, and in view of the wide variety of diseases that may be treated or prevented with compounds having potent microtubule stabilization effects, and the high degree of unmet medical need represented within this variety of diseases, it is desirable to synthesize new compounds with diverse structures that may have improved biological activity profiles for the treatment of one or more indications and to generate tagged taccalonolide probes that retain functionality, both for use as a biological probe and as a means to target the taccalonolide to the desired site of action. These needs and others are met by the present invention.

### SUMMARY

In accordance with the purpose(s) of the invention, as embodied and broadly described herein, the invention, in one aspect, relates to taccalonolide microtubule stabilizers useful as cellular probes (e.g., for the detection, visualization, and/or quantification of a target). Such compounds are also useful in the treatment of hyperproliferative disorders including, but not limited to, cardiovascular diseases such as, for example, coronary heart disease, stroke, hypertensive heart disease, inflammatory heart disease, and rheumatic heart disease, and cancers such as, for example, sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma).

Disclosed are compounds having a structure represented by a formula:

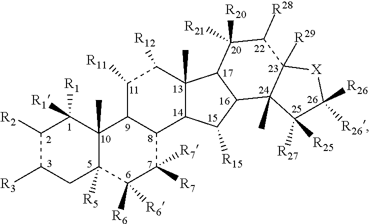

wherein each occurrence of - - - - - - is a single or double covalent bond; wherein X is selected from O, NRx, and C(Rx)2; wherein each occurrence of Rx, when present, is independently selected from hydrogen and C1-C6 alkyl; wherein R1 is selected from hydrogen, halogen, —OH, —CN, —NC, —NCO, —OCN, —NO2, —ONO₂, —ONO, —NO, —N3, —NH2, —NH3, —N═NR31, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR32)₂, —OSO2R33, —C(O)(C1-C12 alkyl), —CO2R34, —C(O)NR35aR35b, —(C1-C12 alkyl)C(O)NR35aR35b, —OC(O)NR35aR35b, —(C1-C12 alkyl)OC(O)NR35aR35b, Cy1, Ar1, (C1-C12 alkyl)Ar1, and —OAr1; wherein each occurrence of R31, R32, R34, R35a, and R35b, when present, is independently selected from hydrogen and C1-C12 alkyl; wherein each occurrence of R33, when present, is independently selected from hydrogen, C1-C12 alkyl, and monocyclic aryl monosubstituted with a methyl group; wherein each occurrence of Cy1, when present, is heterocycloalkyl substituted with 0, 1, 2, or 3 groups independently selected from halogen, —OH, —NH2, C1-C4 alkoxy, C1-C4 hydroxy, C1-C4 aminoalkyl, C1-C4 alkylamino, and (C1-C4)(C1-C4) dialkylamino; wherein each occurrence of Ar1, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH2, C1-C4 alkoxy, C1-C4 hydroxy, C1-C4 aminoalkyl, C1-C4 alkylamino, and (C1-C4)(C1-C4) dialkylamino; and wherein R1′ is hydrogen; or wherein each of R1 and R1′ together comprise ═O or ═NR36; wherein each occurrence of R36, when present, is independently selected from hydrogen and C1-C12 alkyl; wherein each of R2 and R3 is independently selected from hydrogen, —OH, C1-C12 hydroxy, and halogen; or wherein each of R2 and R3 together comprise —O—; wherein R5 is selected from hydrogen, —OH, —NH2, C1-C9 alkyl, C1-C9 hydroxy, C1-C9 alkoxy, C1-C9 aminoalkyl, C1-C6 alkylamino, and (C1-C6)(C1-C6) dialkylamino; or wherein R5 is absent; wherein each of R6 and R6′ is independently selected from hydrogen, halogen, —OH, —CN, —NC, —NCO, —OCN, —NO2, —ONO2, —ONO, —NO, —N3, —NH2, —NH3, —N═NR41, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR32)₂, —OSO2R33, —CO2R34, —C(O)NR35aR35b, —(C1-C30 alkyl)C(O)NR35aR35b, —OC(O)NR35aR35b, —(C1-C30 alkyl)OC(O)NR35aR35b, Cy1, Ar1, —(C1-C30 alkyl)Ar1, —OAr1, —OC(O)(C1-C30 alkyl), —OC(O)Ar2, —OC(O)(C1-C30 alkyl)Ar2, —OC(O)Ar3, —OC(O)(C1-C30 alkyl)NR42C(O)Ar3, —OC(O)(C1-C30 alkyl)OC(O)Ar3, —OC(O)R40, —NR41C(O)(C1-C30 alkyl), —NR41C(O)Ar2, —NR41C(O)(C1-C30 alkyl)Ar2, —NR41C(O)Ar3, —NR41C(O)(C1-C30 alkyl)OC(O)Ar3, —NR41C(O)(C1-C30 alkyl)NR42C(O)Ar3, and —NR41C(O)R40; wherein each occurrence of R40, when present, is independently a C1-C30 alkyl functionalized with a group selected from —N3, —SH, —OH, —NH2, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide; wherein each occurrence of R41 and R42, when present, is independently selected from hydrogen and C1-C12 alkyl; wherein each occurrence of Ar2, when present, is independently selected from aryl and heteroaryl, and is substituted with 0, 1, 2, or 3 groups independently selected from halogen, —OH, —NH2, C1-C4 alkoxy, C1-C4 hydroxy, C1-C4 aminoalkyl, C1-C4 alkylamino, (C1-C4)(C1-C4) dialkylamino, —(C1-C12 alkyl)Ar4, and Ar4; wherein each occurrence of Ar4, when present, is a structure represented by a formula selected from:

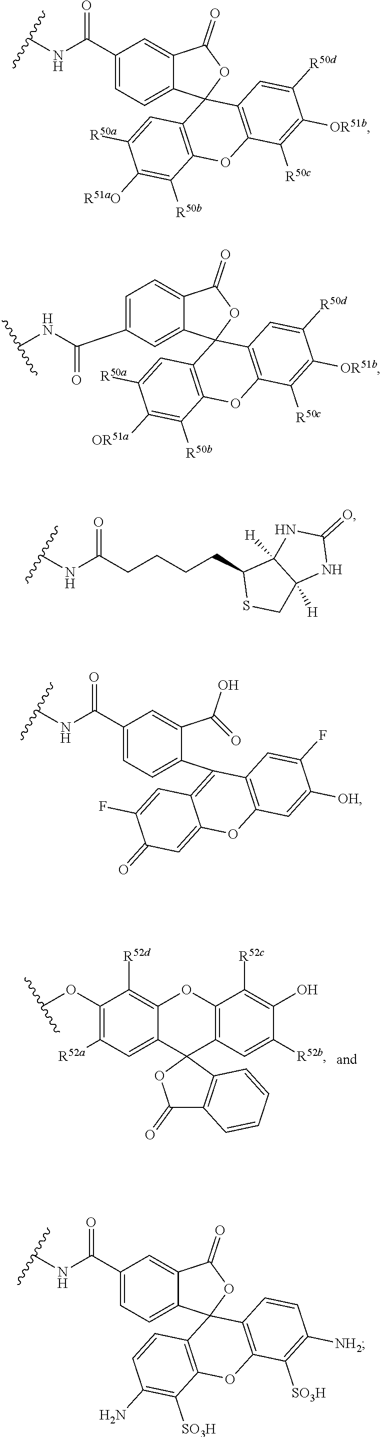

wherein each of R50a, R50b, R50c, and R50d, when present, is independently selected from hydrogen, —F, and —Cl; wherein each of R51a and R51b, when present, is independently selected from hydrogen and —C(O)(C1-C12 alkyl); wherein each of R52a, R52b, R52c, and R52d, when present, is independently selected from hydrogen, —F, and —Cl; wherein each occurrence of Ar3, when present, is a structure represented by a formula selected from:

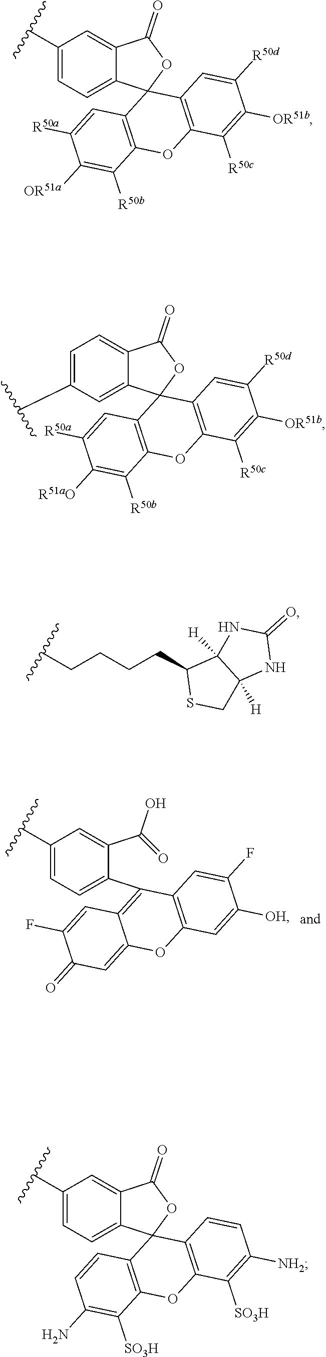

or wherein one of R6 and R6′ is absent; wherein R7 is selected from hydrogen, —OH, C1-C30 hydroxy, C1-C30 alkoxy, —OC(O)(C1-C30 alkyl), and —OC(O)NR35aR35b, and wherein R7′ is selected from hydrogen, —OH, C1-C30 hydroxy, C1-C30 alkoxy, and —OC(O)(C1-C30 alkyl); or wherein each of R7 and R7′ together comprise ═O; or wherein one of R7 and R7′ is absent; wherein each of R11 and R12 is independently selected from hydrogen, —OH, C1-C8 hydroxy, C1-C6 alkyl, C1-C8 alkoxy, and —OC(O)(C1-C8 alkyl); wherein R15 is selected from hydrogen, —OH, C1-C30 hydroxy, C1-C30 alkyl, C1-C30 alkoxy, —OC(O)(C1-C30 alkyl), —OC(O)NR35aR35b, —OC(O)Ar2, —OC(O)(C1-C4 alkyl)Ar2, and —OC(O)(C1-C8 azide); wherein R20 is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R21 is selected from hydrogen and C1-C6 alkyl; wherein R25 is selected from hydrogen, —OH, C1-C8 hydroxy, C1-C8 alkoxy, —OC(O)(C1-18 alkyl), —OC(O)NR35aR35b, —OC(O)Ar5, and —OC(O)(C1-C8 azide); wherein Ar5, when present, is selected from monocyclic 6-membered aryl and anthracene-9,10-dionyl, and is substituted with 0, 1, 2, or 3 groups independently selected from halogen, —OH, —NH2, C1-C4 alkoxy, C1-C4 hydroxy, C1-C4 aminoalkyl, C1-C4 alkylamino, and (C1-C4)(C1-C4) dialkylamino; wherein each of R26 and R26′ is independently selected from hydrogen, —OH, C1-C8 hydroxy, and C1-C8 alkoxy; or wherein each of R26 and R26′ together comprise ═O; wherein R27 is selected from hydrogen and C1-C6 alkyl; wherein each of R28 and R29 is independently selected from hydrogen and halogen; or wherein each of R28 and R29 together comprise —O— or —N(R37)-; wherein R37, when present, is selected from hydrogen, C1-C4 alkyl, —SO2R71, and a structure having a formula:

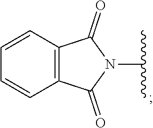 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that at least one of R⁶ and R^{6′} is —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, or —NR⁴¹C(O)R⁴⁰, and provided that when one or both of R²⁸ and R²⁹ is hydrogen then the occurrence of - - - - - - at C-22/C-23 is a double covalent bond, or a pharmaceutically acceptable salt thereof.

Also disclosed are compounds having a structure represented by a formula:

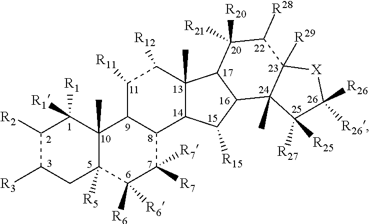

wherein each occurrence of - - - - - - is a single or double covalent bond; wherein X is selected from O, NR^{x} and C(RV)₂; wherein each occurrence of R^{x}, when present, is independently selected from hydrogen and C1-C6 alkyl; wherein R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹; wherein each occurrence of R³¹, R³², R³⁴, R^{35a}, and R^{35b}, when present, is independently selected from hydrogen and C1-C12 alkyl; wherein each occurrence of R³³, when present, is independently selected from hydrogen, C1-C12 alkyl, and monocyclic aryl monosubstituted with a methyl group; wherein each occurrence of Cy1, when present, is heterocycloalkyl substituted with 0, 1, 2, or 3 groups independently selected from halogen, —OH, —NH2, C1-C4 alkoxy, C1-C4 hydroxy, C1-C4 aminoalkyl, C1-C4 alkylamino, and (C1-C4)(C1-C4) dialkylamino; wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and wherein R^{1′} is hydrogen; or wherein each of R¹ and R^{1′} together comprise ═O or ═NR³⁶; wherein each occurrence of R³⁶, when present, is independently selected from hydrogen and C1-C12 alkyl; wherein each of R² and R³ is independently selected from hydrogen, —OH, C1-C12 hydroxy, and halogen; or wherein each of R₂ and R₃ together comprise —O—; wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; wherein each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr¹, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)R⁴⁰, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰; wherein each occurrence of R⁴⁰, when present, is independently a C1-C30 alkyl functionalized with a group selected from —SH, —NH₂, —NHOH, terminal alkyne, haloacetyl, maleimide, isothiocyanate, N-hydroxysuccinimde, succinimidyl ester, tetrafluorophenyl ester, sulfodichlorophenol ester, and hydrazide; wherein each occurrence of R⁴¹ and R⁴², when present, is independently selected from hydrogen and C1-C12 alkyl; wherein each occurrence of Ar2, when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴; wherein each occurrence of Ar⁴, when present, is a structure represented by a formula:

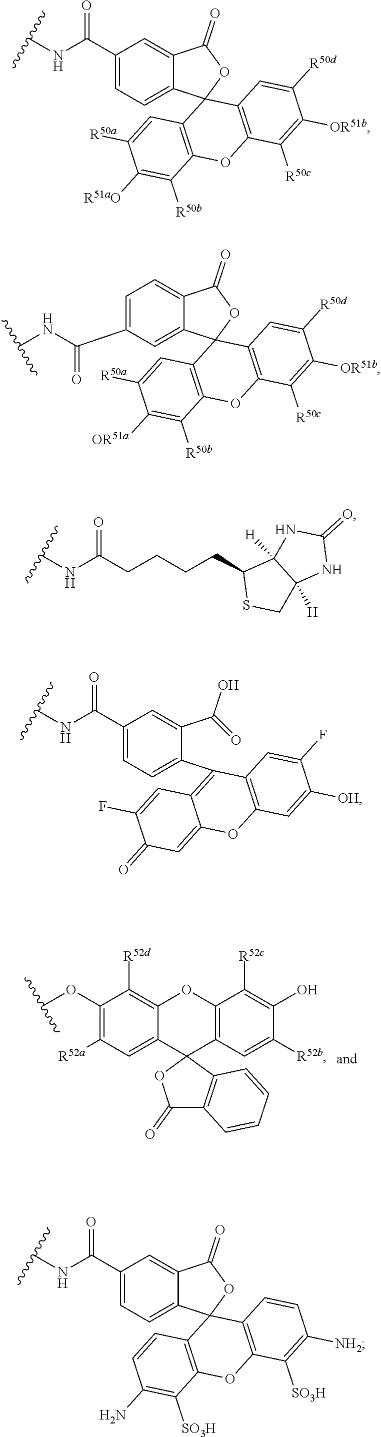

wherein each of R^{50a}, R^{50b}, R^{50c}, and R^{50d}, when present, is independently selected from hydrogen, —F, and —Cl; wherein each of R^{51a} and R^{51b}, when present, is independently selected from hydrogen and —C(O)(C1-C12 alkyl); wherein each of R^{52a}, R^{52b}, R^{52c}, and R^{52d}, when present, is independently selected from hydrogen, —F, and —Cl; wherein each occurrence of Ar³, when present, is a structure represented by a formula selected from:

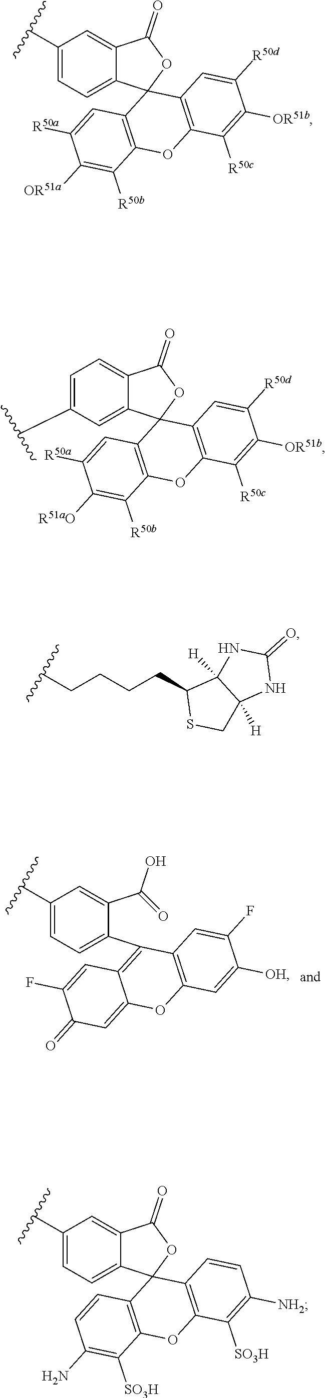

or wherein one of R⁶ and R^{6′} is absent; wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl); or wherein each of R⁷ and R^{7′} together comprise ═O; or wherein one of R⁷ and R^{7′} is absent; wherein each of R¹¹ and R¹² is independently selected from hydrogen, —OH, C1-C8 hydroxy, C1-C6 alkyl, C1-C8 alkoxy, and —OC(O)(C1-C8 alkyl); wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide); wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R²¹ is selected from hydrogen and C1-C6 alkyl; wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide); wherein Ar¹, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each of R²⁶ and R^{26′} is independently selected from hydrogen, —OH, C1-C8 hydroxy, and C1-C8 alkoxy; or wherein each of R²⁶ and R^{26′} together comprise ═O; wherein R²⁷ is selected from hydrogen and C1-C6 alkyl; wherein each of R²⁸ and R²⁹ is independently selected from hydrogen and halogen; or wherein each of R²⁸ and R²⁹ together comprise —O— or —N(R³⁷)—; wherein R³⁷, when present, is selected from hydrogen, C1-C4 alkyl, —SO₂R⁷¹, and a structure having a formula:

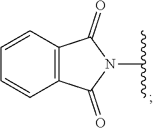 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that at least one of R⁶ and R^{6′} is —OC(O)R⁴⁰ or —NR⁴¹C(O)R⁴⁰, and provided that when one or both of R²⁸ and R²⁹ is hydrogen then the occurrence of at C-22/C-23 is a double covalent bond, or a pharmaceutically acceptable salt thereof.

Also disclosed are compounds having a structure represented by a formula:

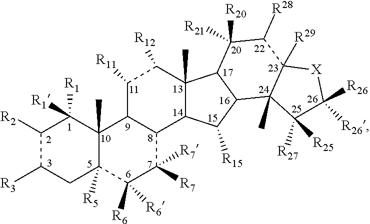

wherein each occurrence of - - - - - - is a single or double covalent bond; wherein X is selected from O, NR^{x} and C(R^{x})₂; wherein each occurrence of R^{x}, when present, is independently selected from hydrogen and C1-C6 alkyl; wherein R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹; wherein each occurrence of R³¹, R³², R³⁴, R^{35a}, and R^{35b}, when present, is independently selected from hydrogen and C1-C12 alkyl; wherein each occurrence of R³³, when present, is independently selected from hydrogen, C1-C12 alkyl, and monocyclic aryl monosubstituted with a methyl group; wherein each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and wherein R^{1′} is hydrogen; or wherein each of R¹ and R^{1′} together comprise ═O or ═NR³⁶; wherein each occurrence of R³⁶, when present, is independently selected from hydrogen and C1-C12 alkyl; wherein each of R² and R³ is independently selected from hydrogen, —OH, C1-C12 hydroxy, and halogen; or wherein each of R₂ and R₃ together comprise —O—; wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl); or wherein each of R⁷ and R^{7′} together comprise ═O; or wherein one of R⁷ and R^{7′} is absent; wherein each of R⁸ and R^{8′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr¹, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, —NR⁴¹C(O)(C1-C30 alkyl)-L-Z, —NR⁴¹C(O)(C1-C30 alkyl)-L-(C1-C30 alkyl)-Z, and —NR⁴¹C(O)-L-(C1-C30 alkyl)-Z; wherein L is a linker; wherein Z is selected from an antibody, an antibody fragment, a vitamin, a hormone, a carbohydrate, a molecular ligand, an aptamer, a non-antibody protein, a peptide, a nucleic acid, a fluorophore, and a drug; wherein each occurrence of R⁴¹ and R⁴², when present, is independently selected from hydrogen and C1-C12 alkyl; wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴; wherein each occurrence of Ar⁴, when present, is a structure represented by a formula:

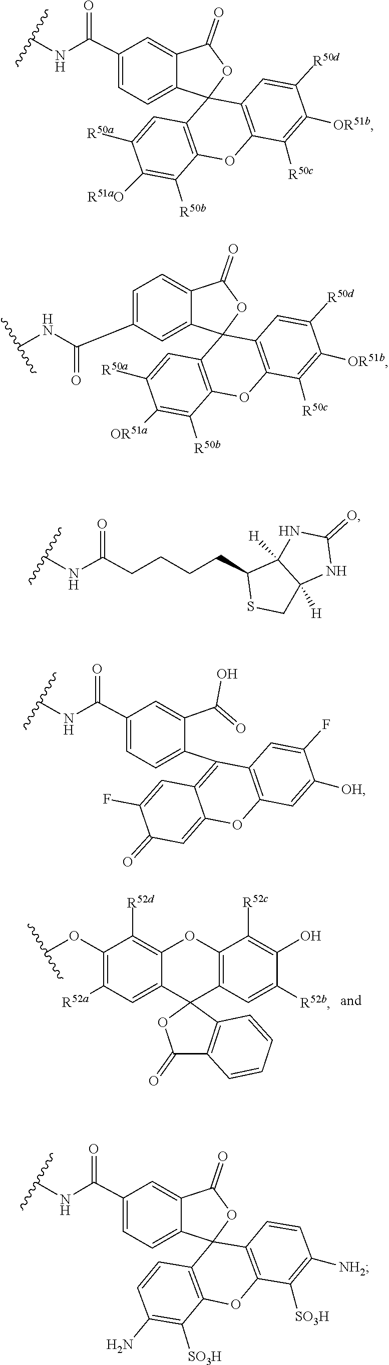

wherein each of R^{50a}, R^{50b}, R^{50c}, and R^{50d}, when present, is independently selected from hydrogen, —F, and —Cl; wherein each of R^{51a} and R^{51b}, when present, is independently selected from hydrogen and —C(O)(C1-C12 alkyl); wherein each of R^{52a}, R^{52b}, R^{52c}, and R^{52d}, when present, is independently selected from hydrogen, —F, and —Cl; wherein each occurrence of Ar³, when present, is a structure represented by a formula selected from:

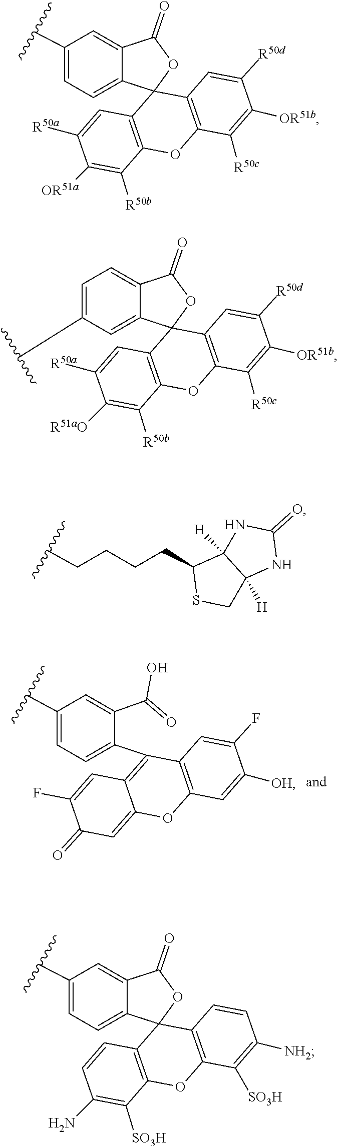

or wherein one of R⁸ and R^{8′} is absent; wherein each of R¹¹ and R¹² is independently selected from hydrogen, —OH, C1-C8 hydroxy, C1-C6 alkyl, C1-C8 alkoxy, and —OC(O)(C1-C8 alkyl); wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide); wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R²¹ is selected from hydrogen and C1-C6 alkyl; wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide); wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each of R²⁶ and R^{26′} is independently selected from hydrogen, —OH, C1-C8 hydroxy, and C1-C8 alkoxy; or wherein each of R²⁶ and R^{26′} together comprise ═O; wherein R²⁷ is selected from hydrogen and C1-C6 alkyl; wherein each of R²⁸ and R²⁹ is independently selected from hydrogen and halogen; or wherein each of R²⁸ and R²⁹ together comprise —O— or N(R³⁷)—; wherein R³⁷, when present, is selected from hydrogen, C1-C4 alkyl, —SO₂R⁷¹, and a structure having a formula:

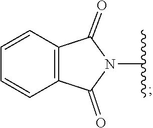 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that one and only one of R⁸ and R^{8′} is —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl)-L-Z, —NR⁴¹C(O)(C1-C30 alkyl)-L-(C1-C30 alkyl)-Z, or —NR⁴¹C(O)-L-(C1-C30 alkyl)-Z, and provided that when one or both of R²⁸ and R²⁹ is hydrogen then the occurrence of - - - - - - at C-22/C-23 is a double covalent bond, or a pharmaceutically acceptable salt thereof.

Also disclosed are compounds having a structure selected from:

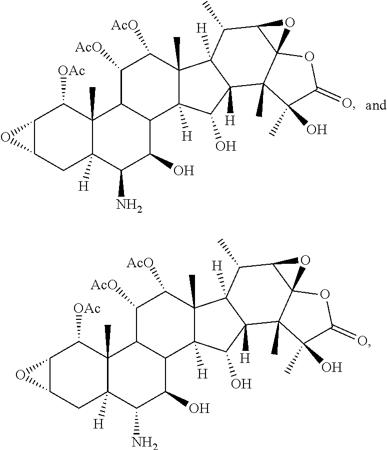 [SMILES: CC(=O)O[C@H]1C2C([C@@H](O)[C@@H](N)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@H](N)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

or a pharmaceutically acceptable salt thereof.

Also disclosed are compounds selected from:

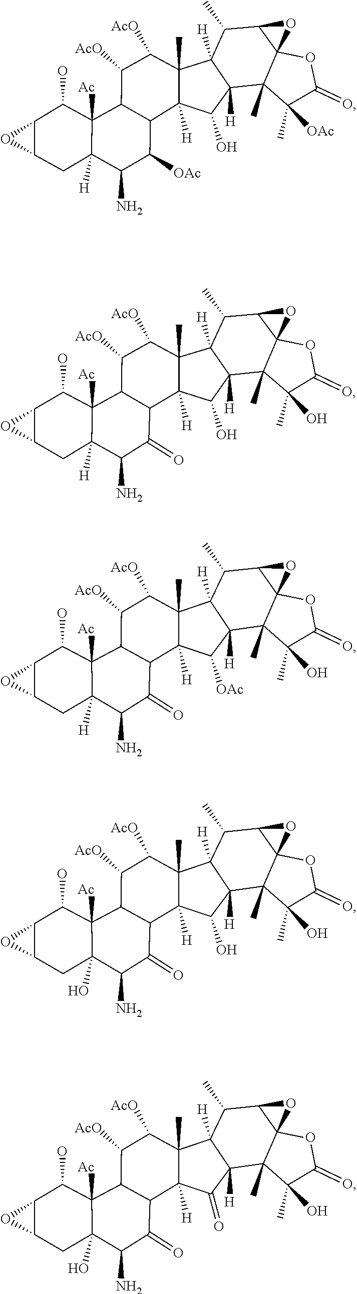

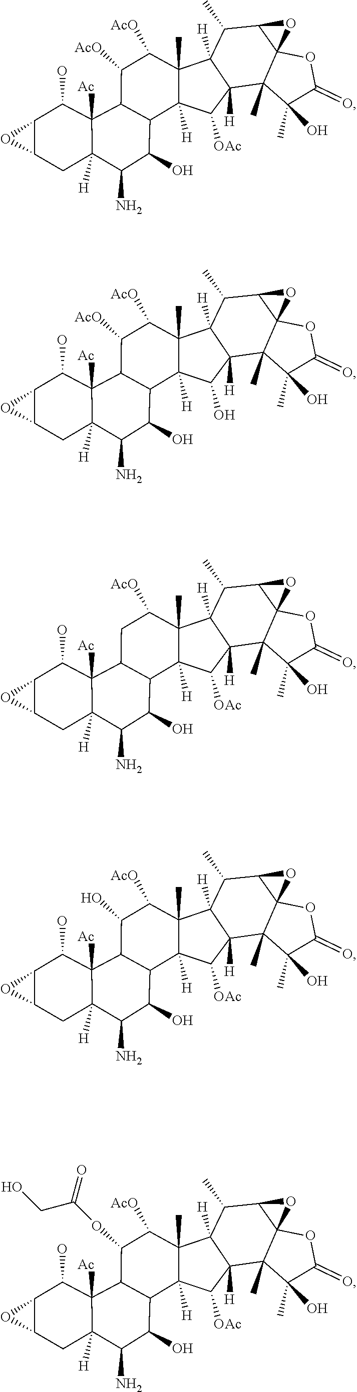

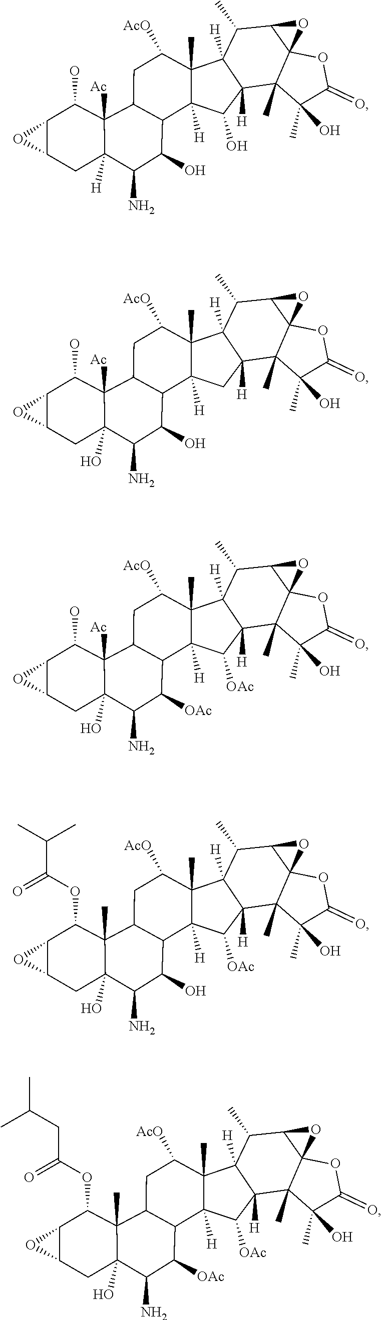

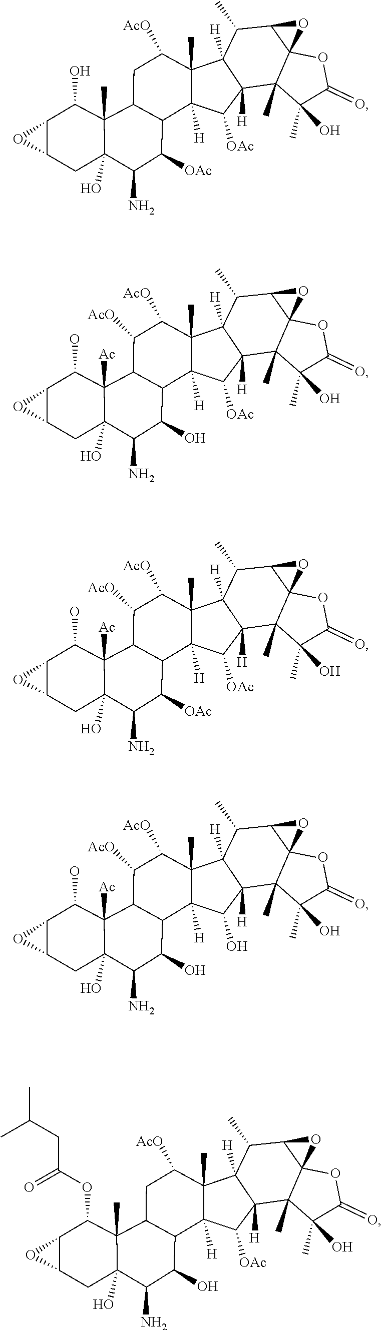

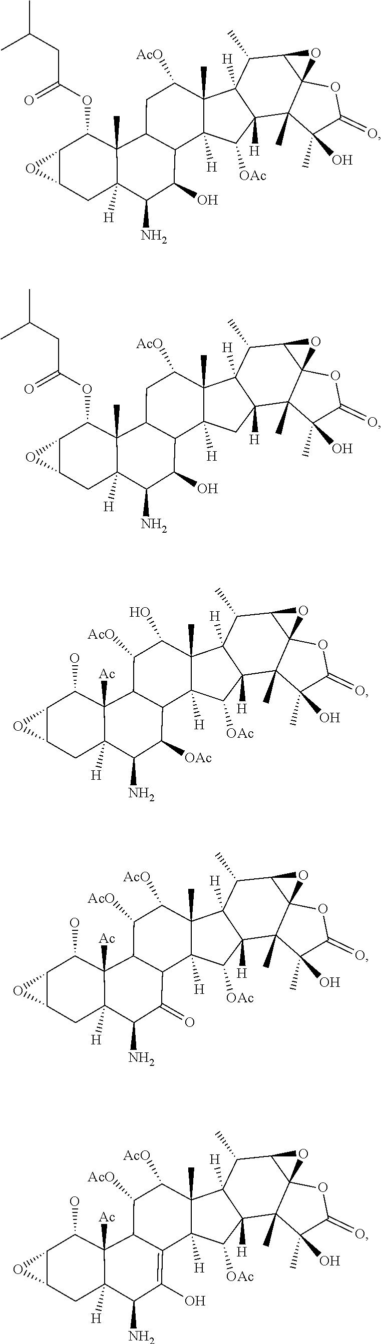

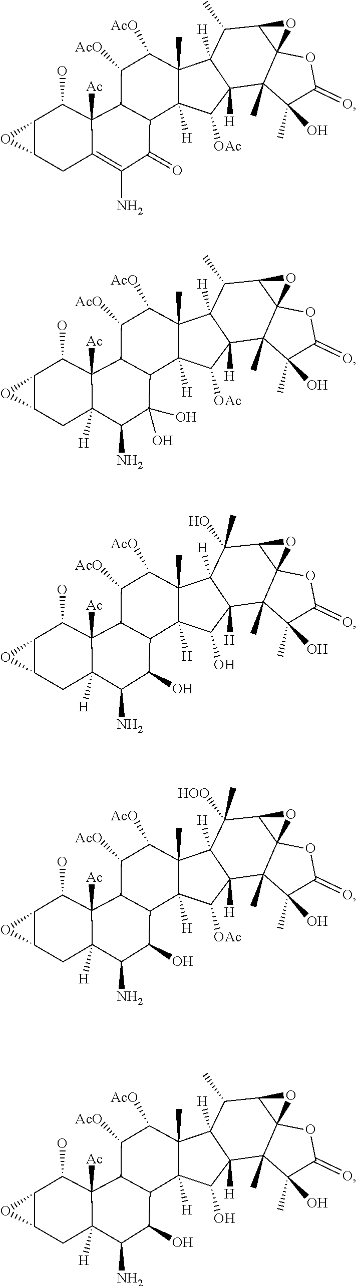

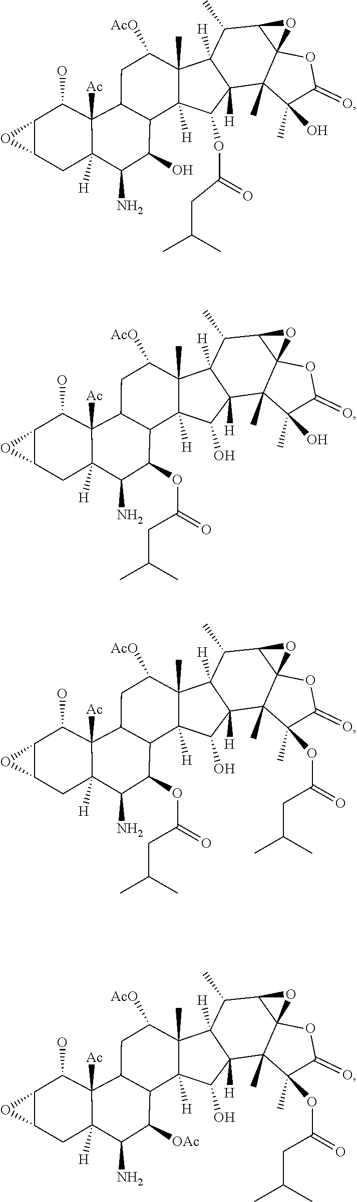

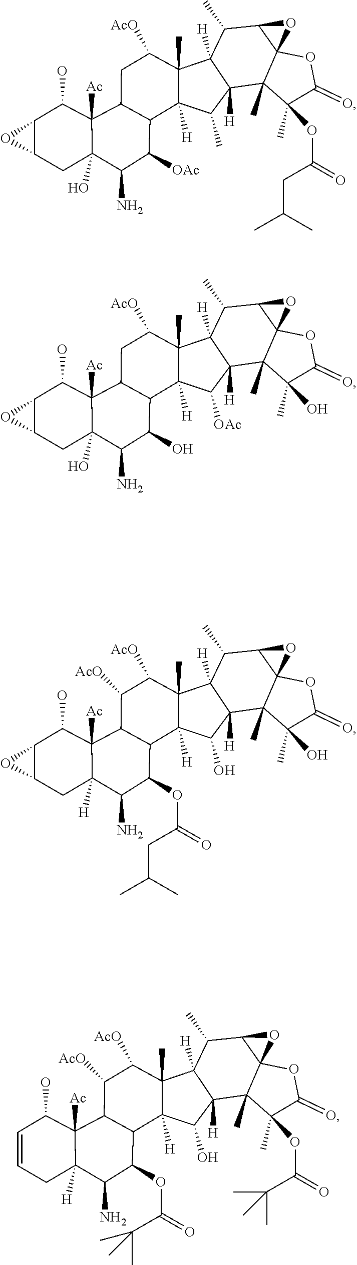

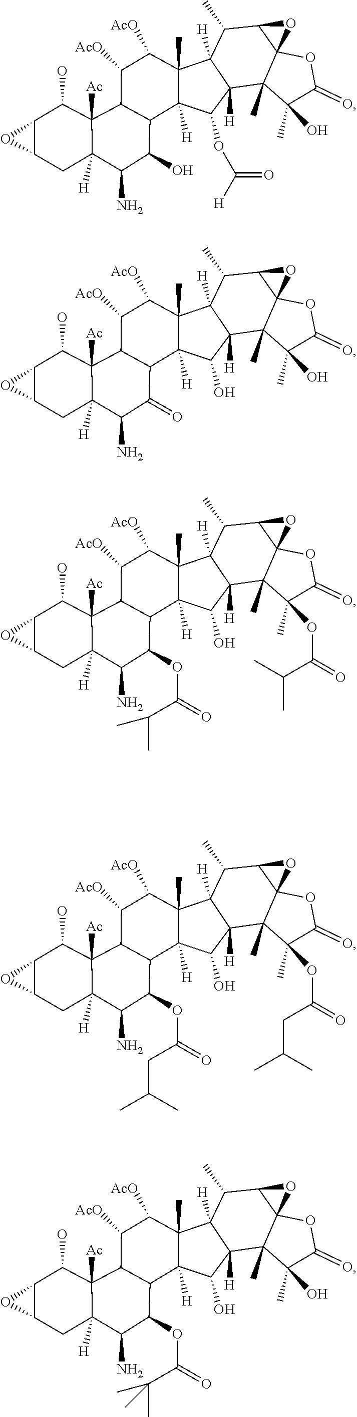

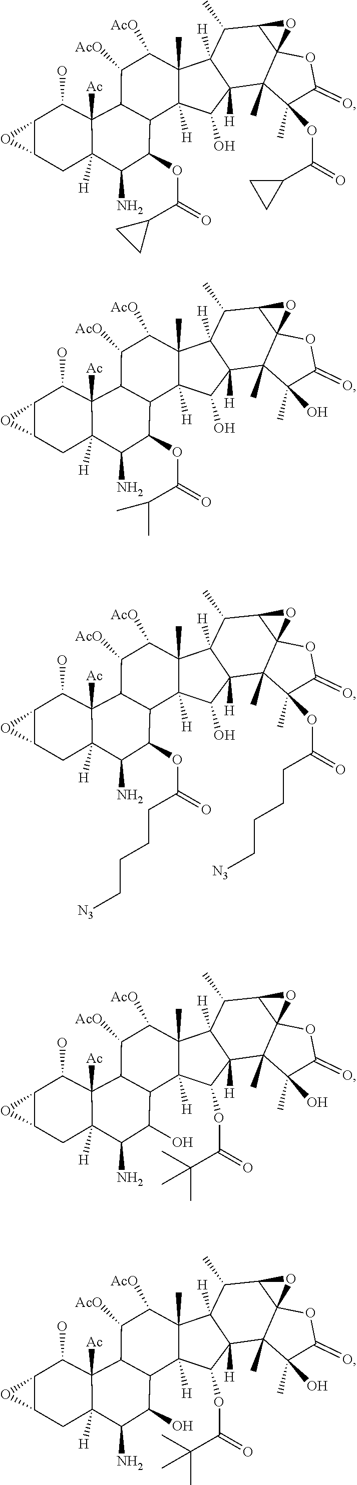

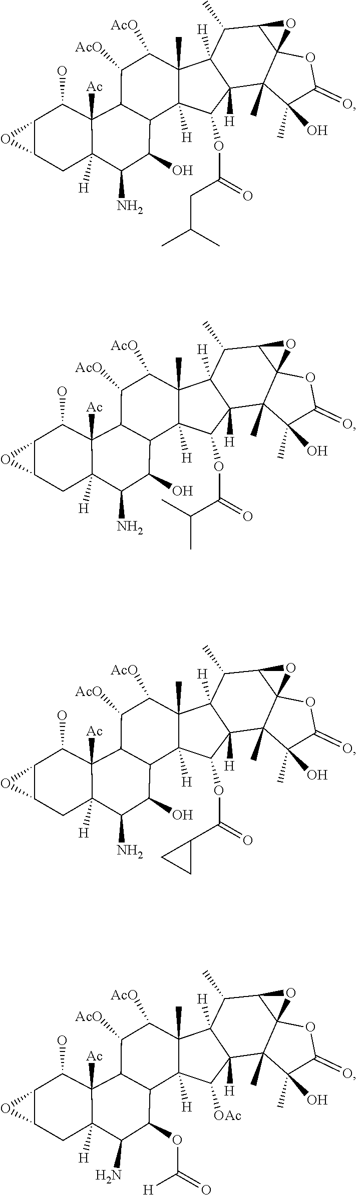

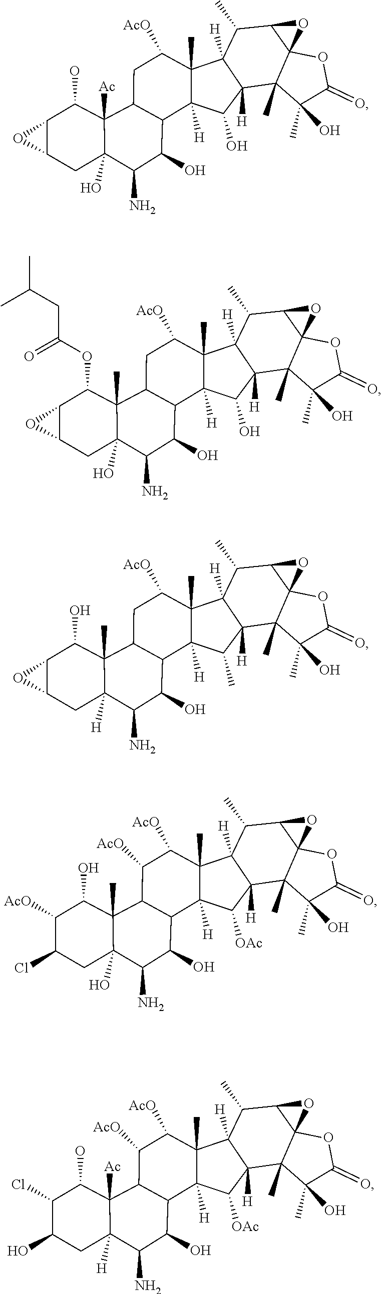

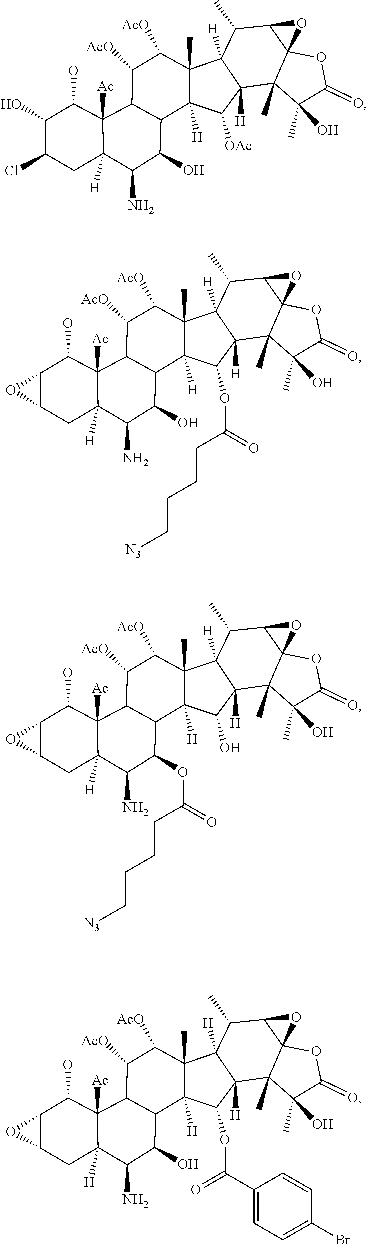

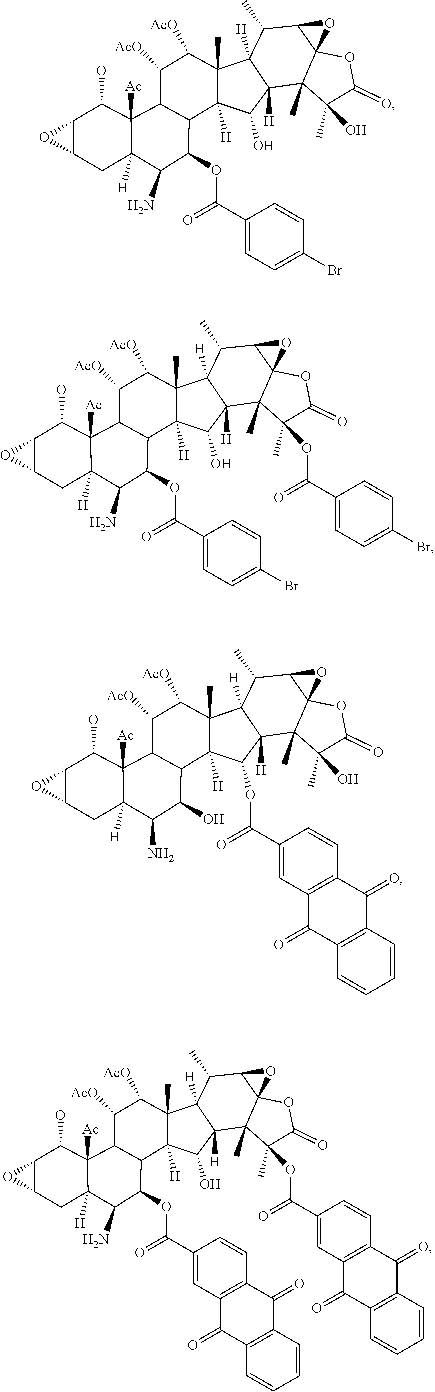

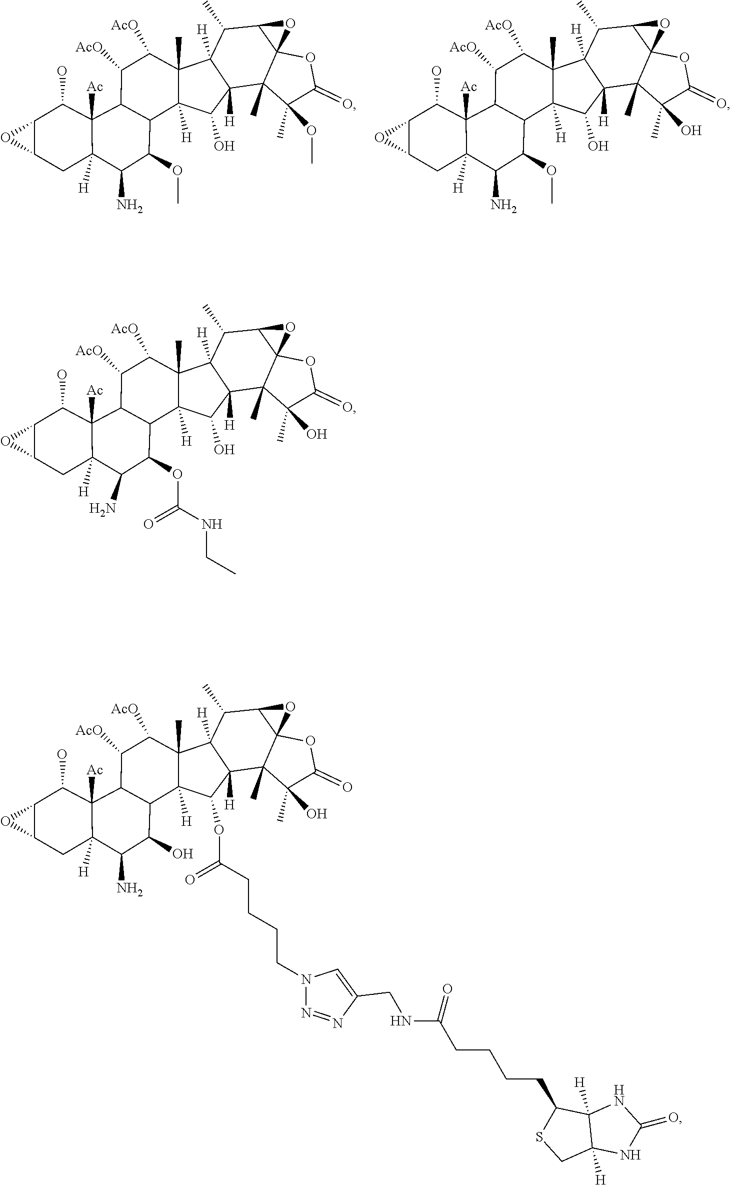

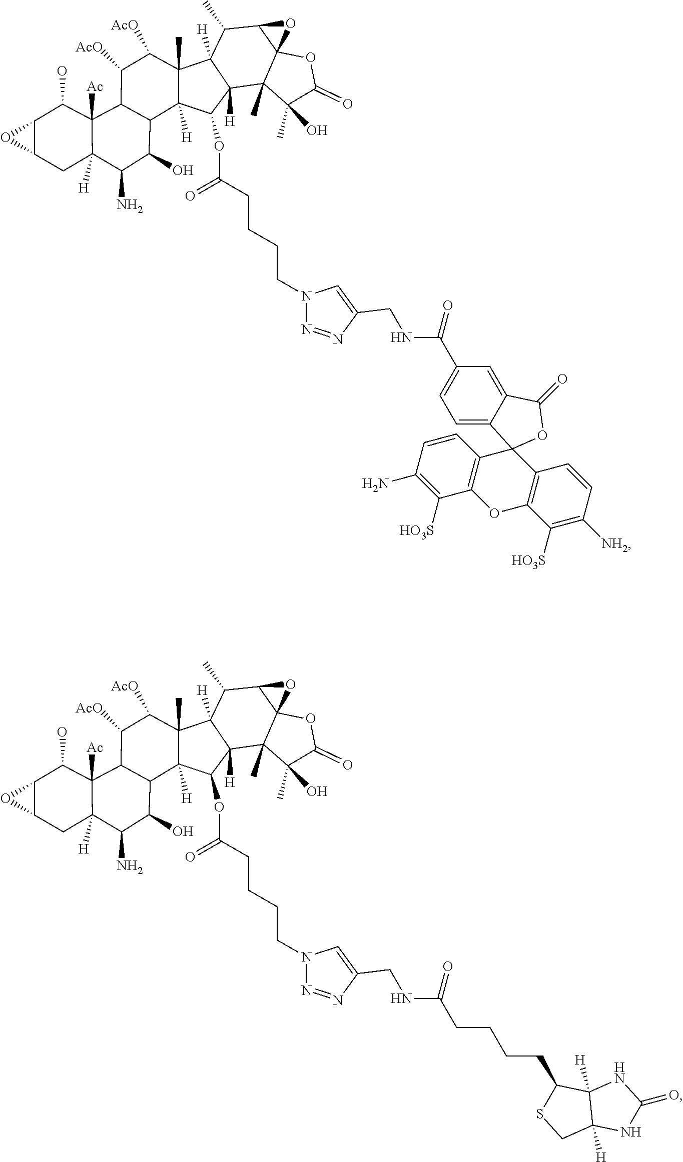

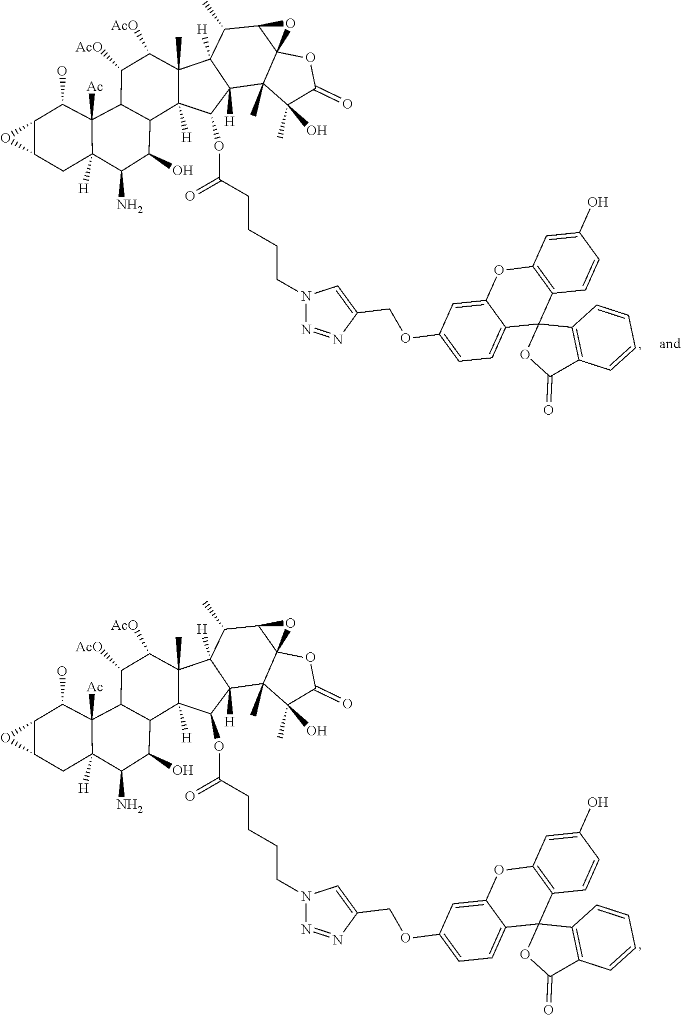 [SMILES: CC(=O)O[C@H]1C2C([C@@H](O)[C@@H](N)[C@H]3C[C@@H]4O[C@@H]4[C@H](O)[C@]23C(C)=O)[C@@H]2[C@@H](OC(=O)CCCCn3cc(COc4ccc5c(c4)Oc4cc(O)ccc4C54OC(=O)c5ccccc54)nn3)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@@H](N)[C@H]3C[C@@H]4O[C@@H]4[C@H](O)[C@]23C(C)=O)[C@@H]2[C@H](OC(=O)CCCCn3cc(COc4ccc5c(c4)Oc4cc(O)ccc4C54OC(=O)c5ccccc54)nn3)[C@@H]3[C@H]([C@H](C)[C@H]4O[C@]45OC(=O)[C@@](C)(O)[C@]35C)[C@@]2(C)[C@H]1OC(C)=O]

or a pharmaceutically acceptable salt thereof.

Also disclosed are compounds selected from:

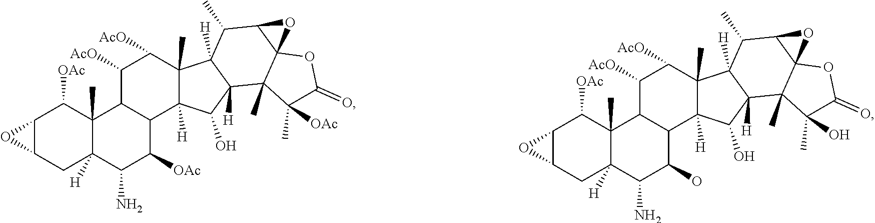 [SMILES: CC(=O)O[C@@H]1C2C([C@H](OC(C)=O)[C@H](OC(C)=O)[C@]3(C)[C@H]4[C@H](C)[C@H]5O[C@]56OC(=O)[C@@](C)(OC(C)=O)[C@]6(C)[C@@H]4[C@H](O)[C@@H]23)[C@]2(C)[C@H](C[C@@H]3O[C@@H]3[C@@H]2OC(C)=O)[C@H]1N ; CC(=O)O[C@H]1C2C([C@@H](O)[C@H](N)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

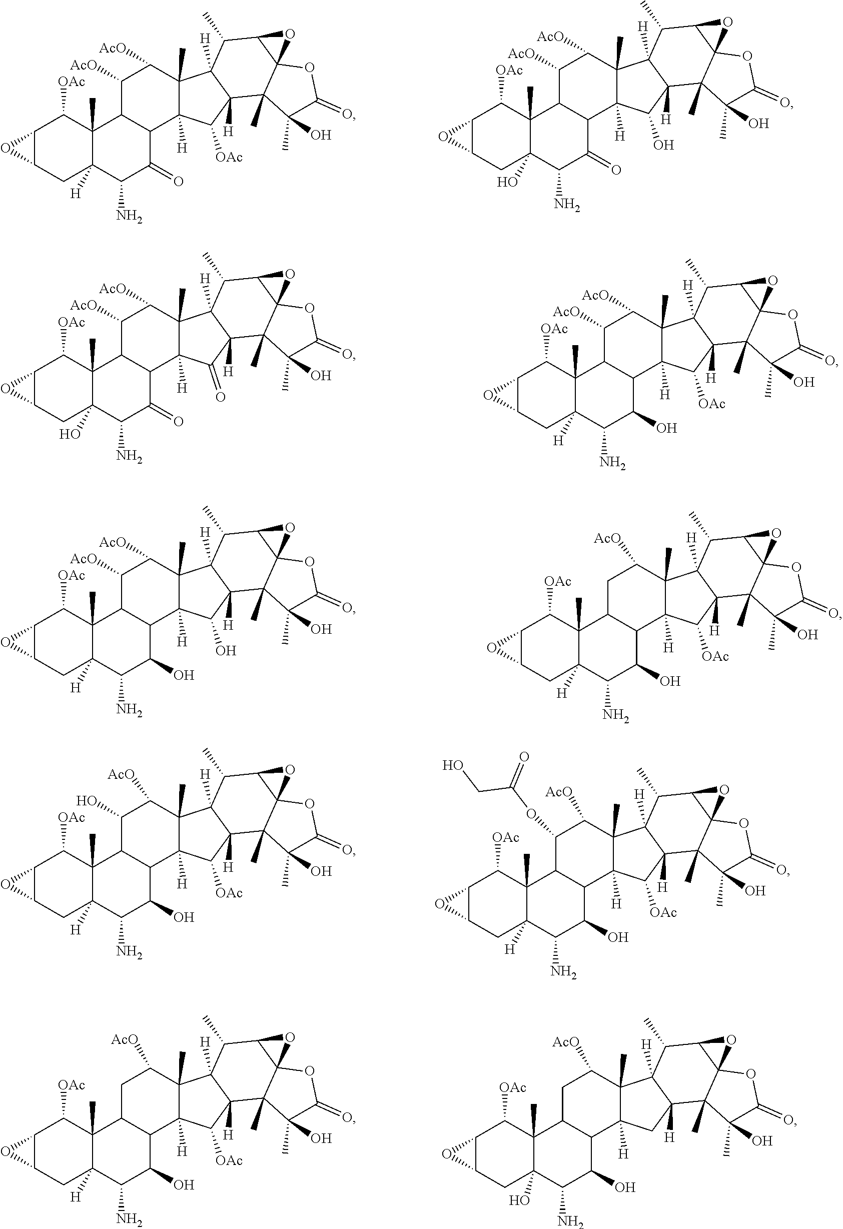

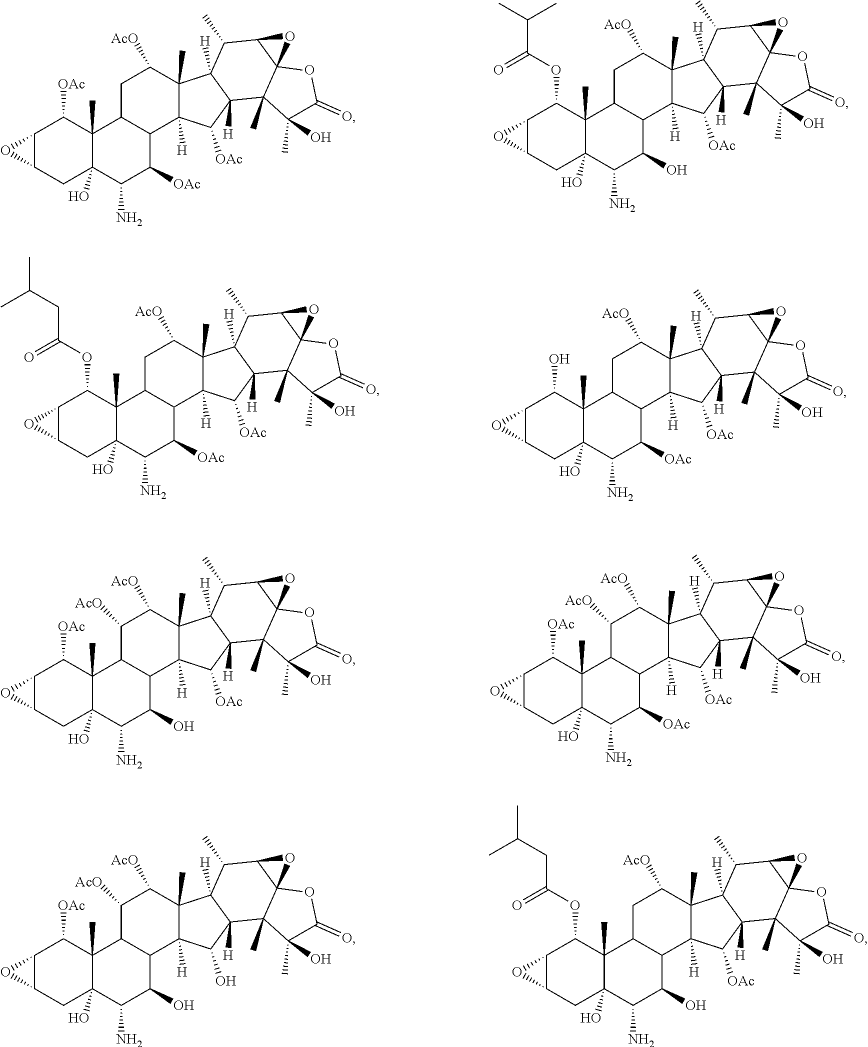

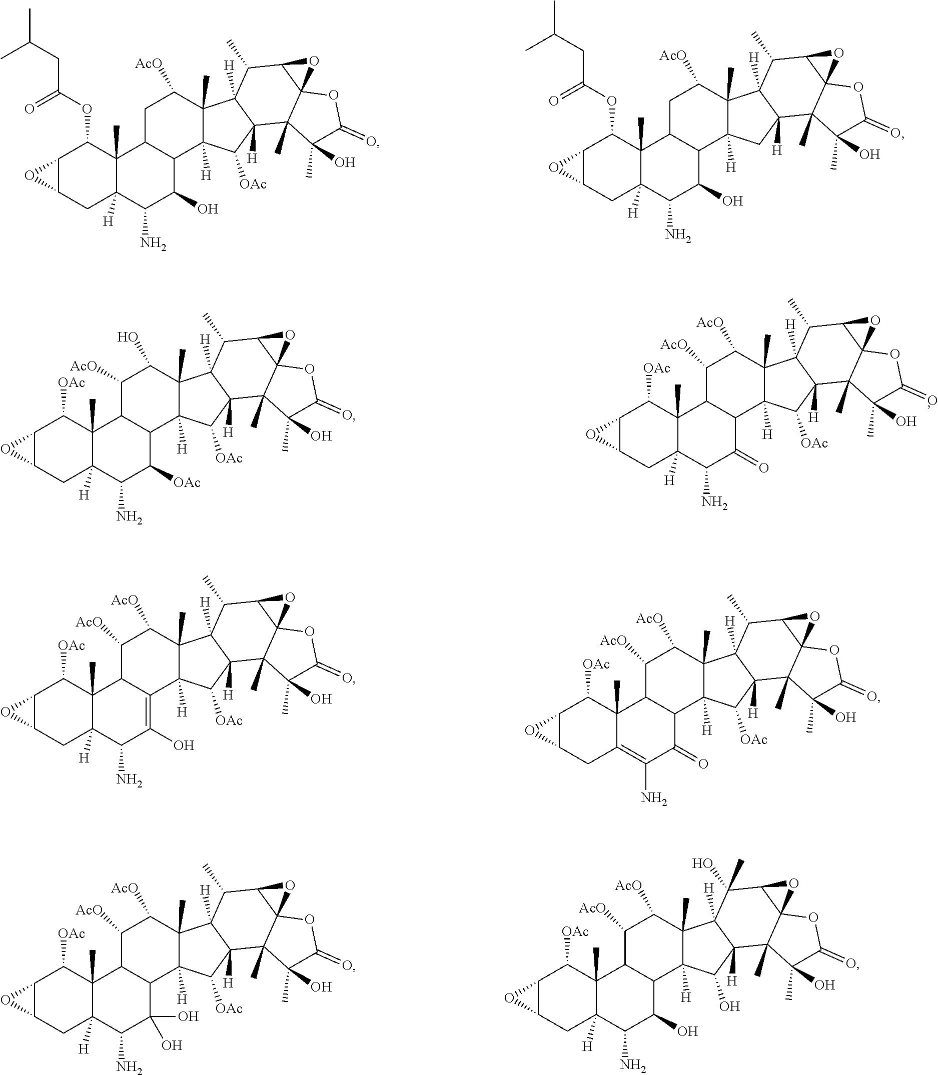

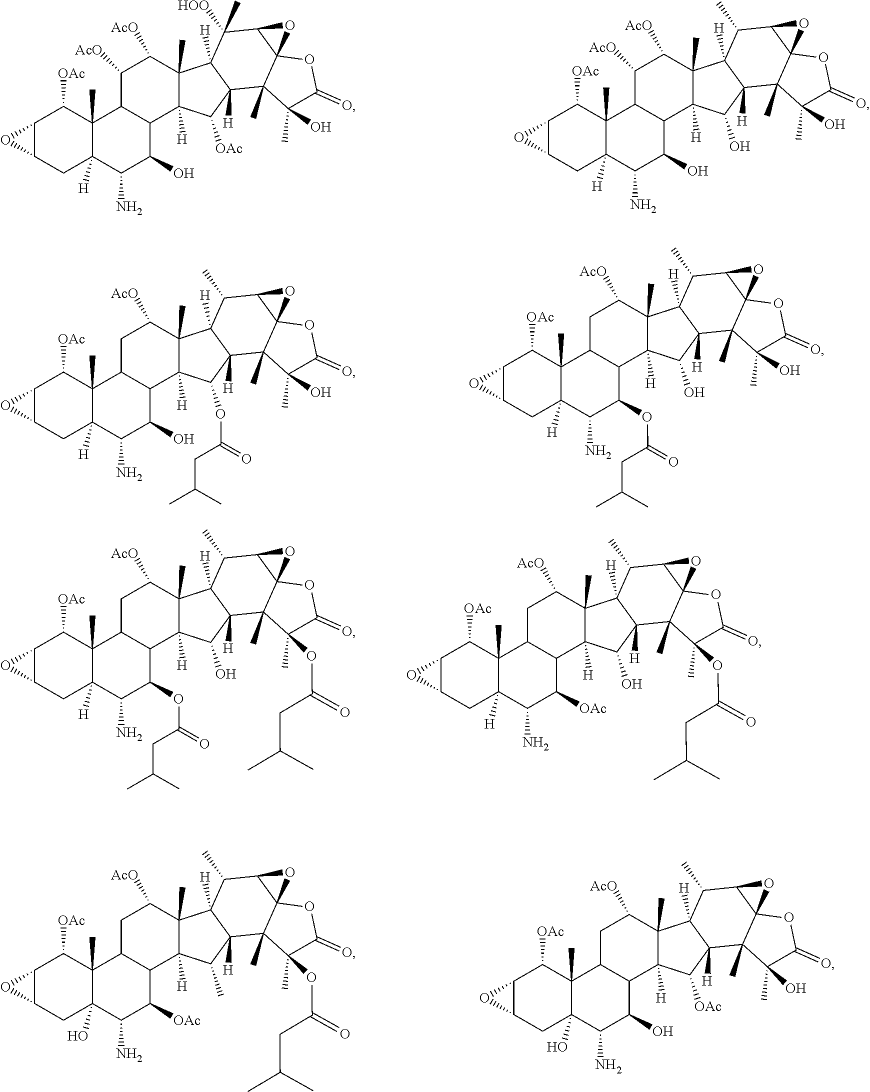

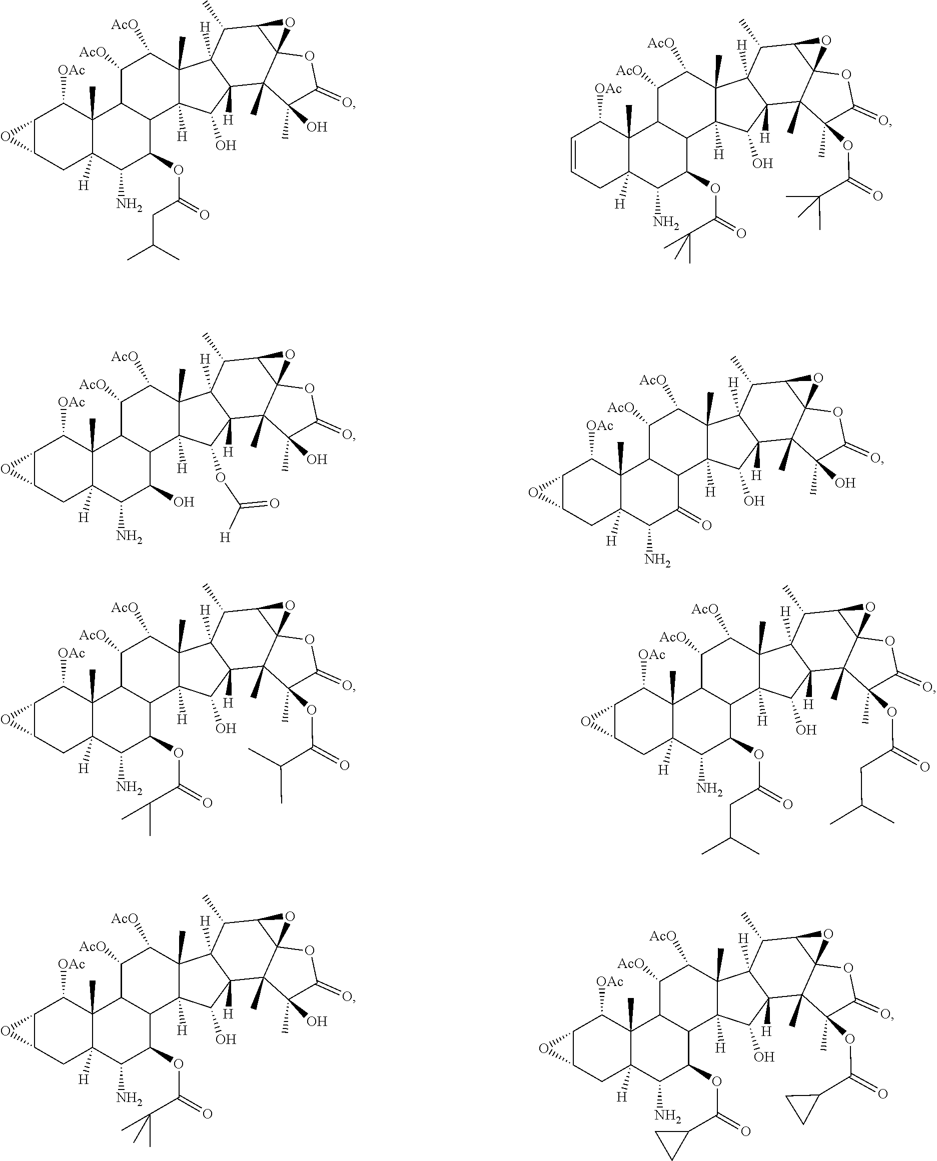

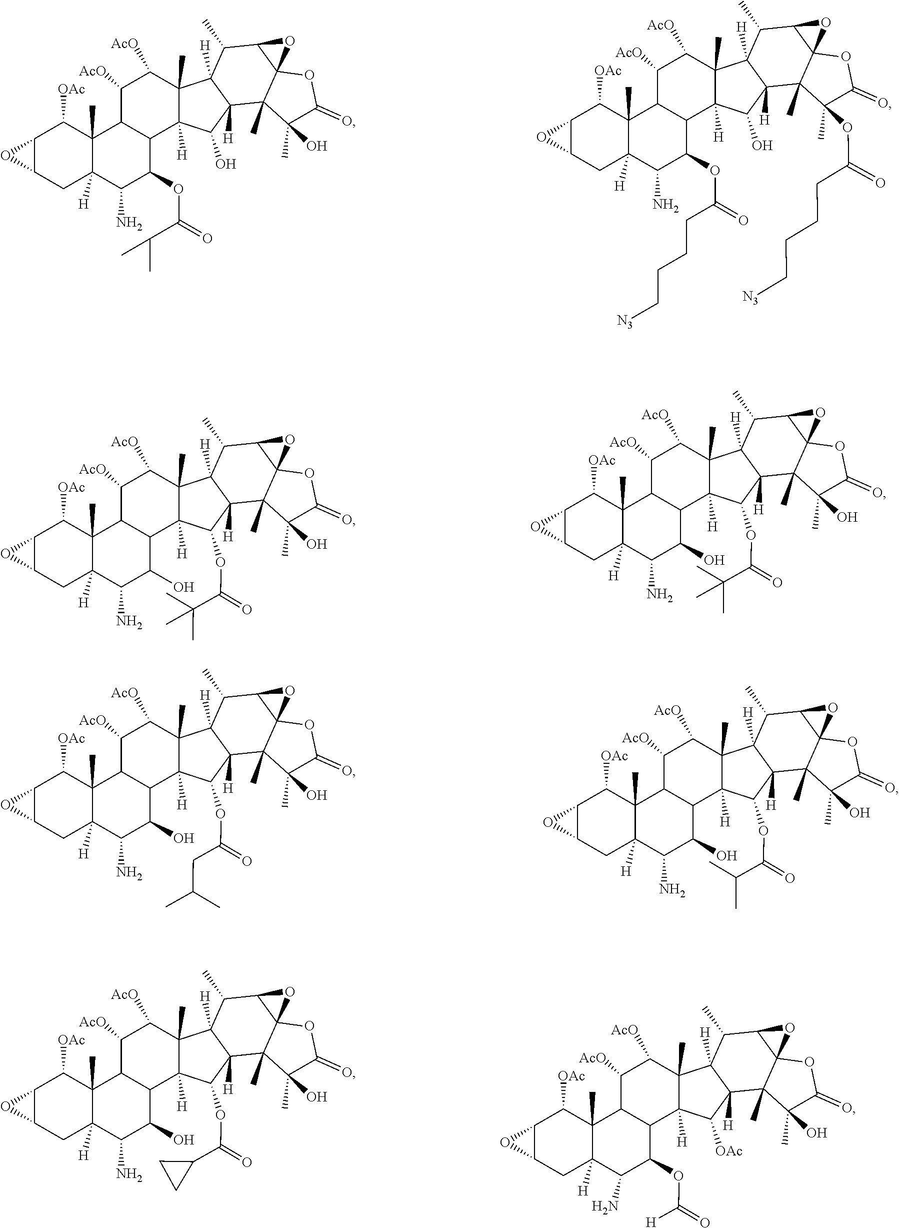

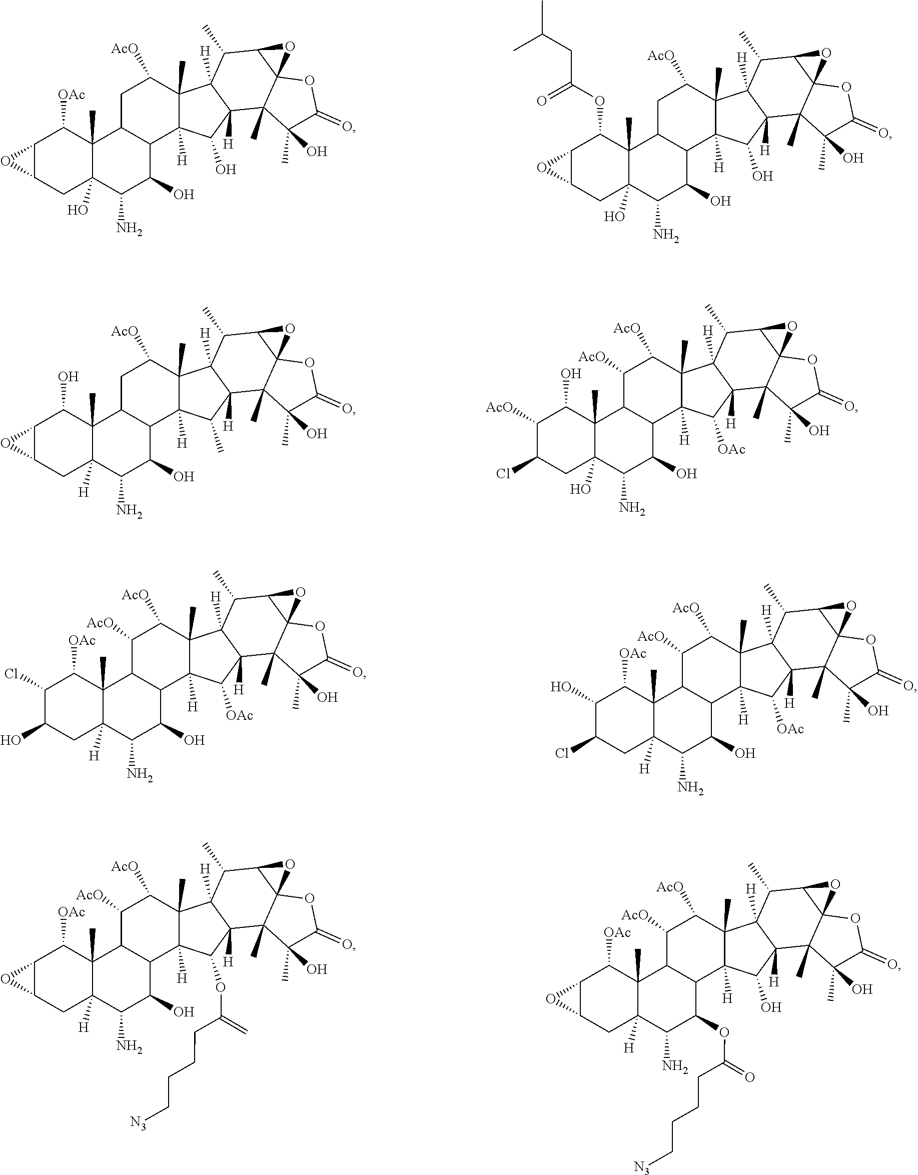

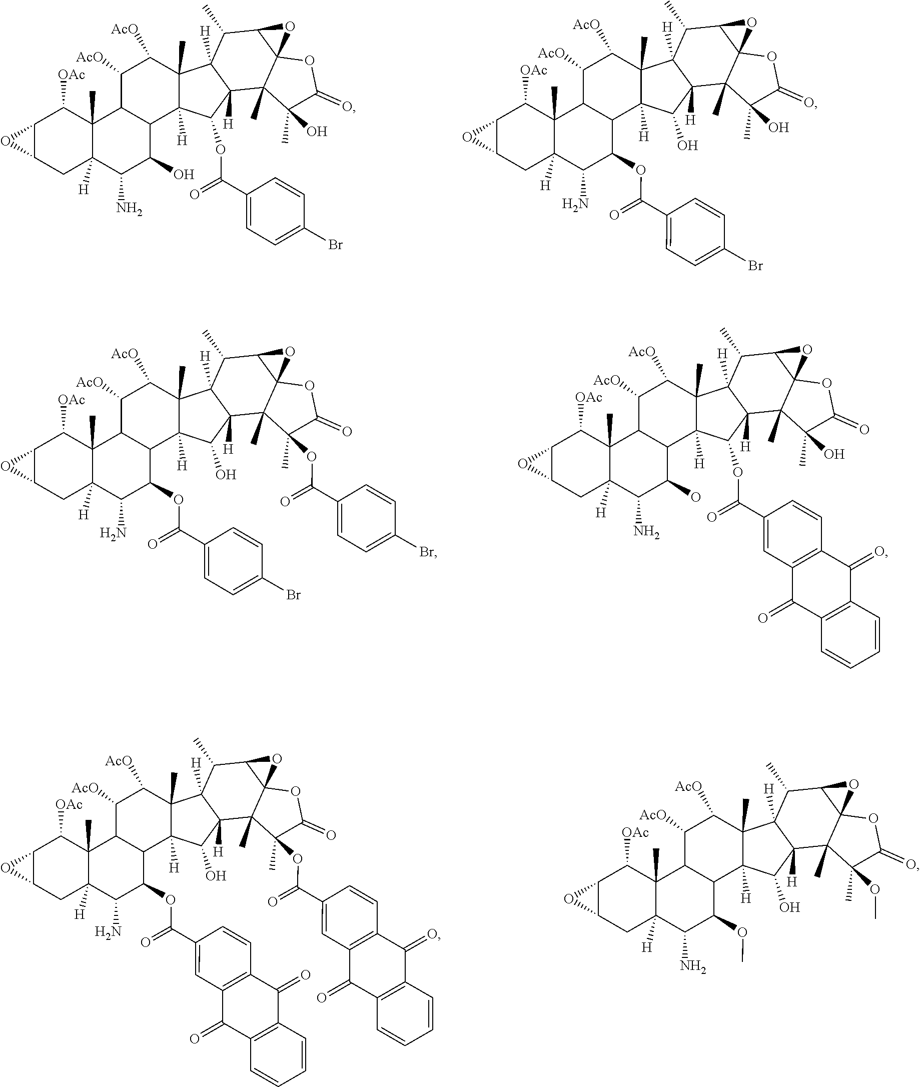

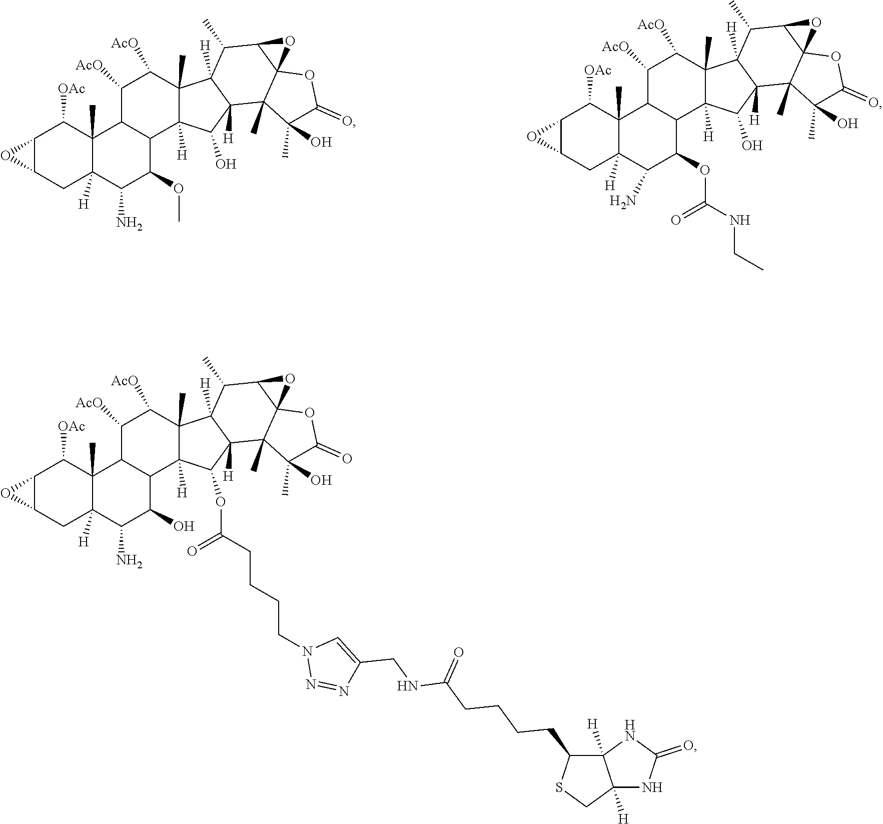

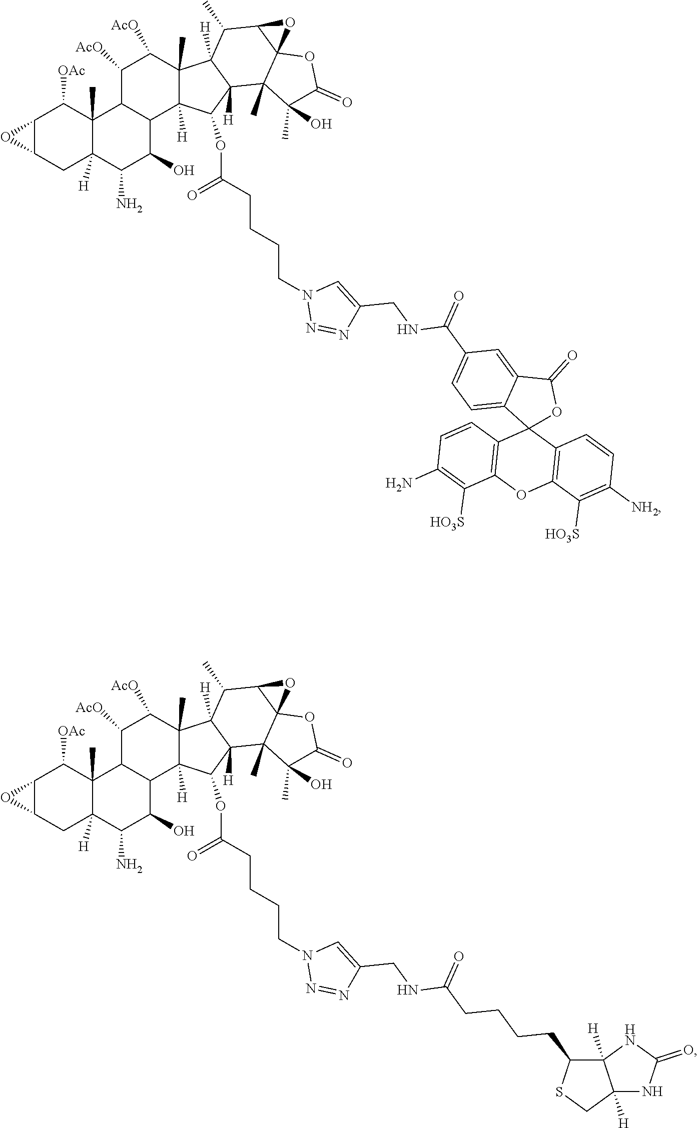

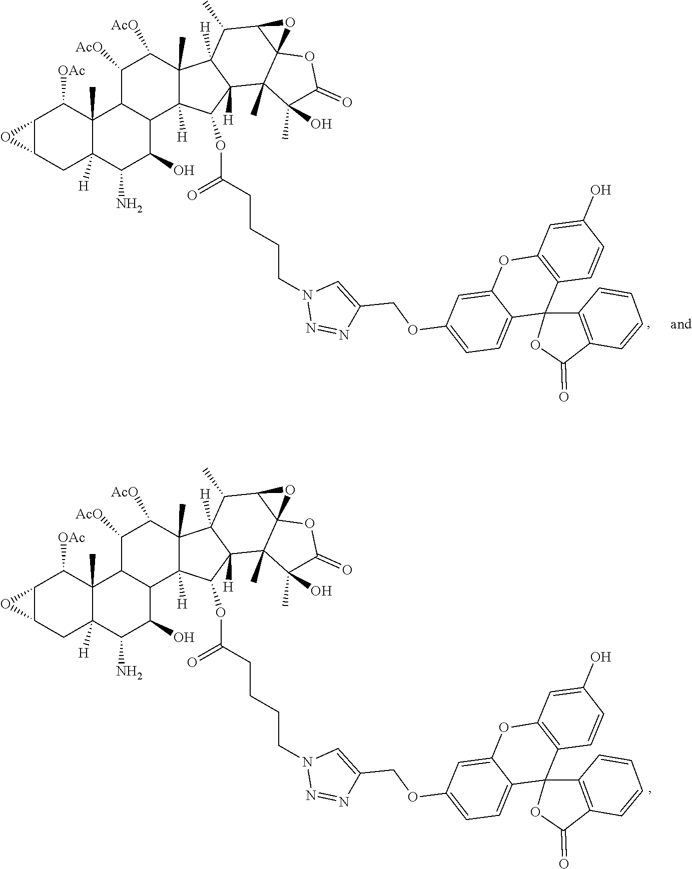 [SMILES: CC(=O)O[C@H]1C2C([C@@H](O)[C@H](N)[C@H]3C[C@@H]4O[C@@H]4[C@H](OC(C)=O)[C@]23C)[C@@H]2[C@@H](OC(=O)CCCCn3cc(COc4ccc5c(c4)Oc4cc(O)ccc4C54OC(=O)c5ccccc54)nn3)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@H](N)[C@H]3C[C@@H]4O[C@@H]4[C@H](OC(C)=O)[C@]23C)[C@@H]2[C@@H](OC(=O)CCCCn3cc(COc4ccc5c(c4)Oc4cc(O)ccc4C54OC(=O)c5ccccc54)nn3)[C@@H]3[C@H]([C@H](C)[C@H]4O[C@]45OC(=O)[C@@](C)(O)[C@]35C)[C@@]2(C)[C@H]1OC(C)=O]

or a pharmaceutically acceptable salt thereof.

Also disclosed are methods of making a disclosed compound.

Also disclosed are pharmaceutical compositions comprising a therapeutically effective amount of a disclosed compound and a pharmaceutically acceptable carrier.

Also disclosed are methods for the treatment of a hyperproliferative disorder in a subject, the method comprising administering to the subject an effective amount of at least one disclosed compound.

Also disclosed are kits comprising at least one disclosed compound and one or more of: (a) at least one agent associated with the treatment of a hyperproliferative disorder; (b) instructions for administering the compound in connection with treating a hyperproliferative disorder; and (c) instructions for treating a hyperproliferative disorder.

Also disclosed are methods of making a conjugated taccalonolide compound having a structure represented by a formula:

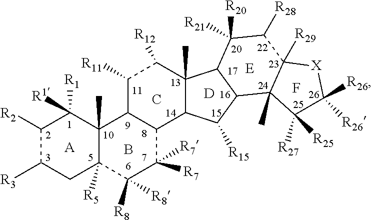

wherein each occurrence of - - - - - - is a single or double covalent bond; wherein X is selected from O, NR^{x} and C(R^{x})₂; wherein each occurrence of R^{x}, when present, is independently selected from hydrogen and C1-C6 alkyl; wherein R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹; wherein each occurrence of R³¹, R³², R³⁴, R^{35a}, and R^{35b}, when present, is independently selected from hydrogen and C1-C12 alkyl; wherein each occurrence of R³³, when present, is independently selected from hydrogen, C1-C12 alkyl, and monocyclic aryl monosubstituted with a methyl group; wherein each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and wherein R^{1′} is hydrogen; or wherein each of R¹ and R^{1′} together comprise ═O or ═NR³⁶; wherein each occurrence of R³⁶, when present, is independently selected from hydrogen and C1-C12 alkyl; wherein each of R² and R³ is independently selected from hydrogen, —OH, C1-C12 hydroxy, and halogen; or wherein each of R₂ and R₃ together comprise —O—; wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; or wherein each of R⁷ and R^{7′} together comprise ═O; or wherein one of R⁷ and R^{7′} is absent; wherein each of R⁸ and R^{8′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr¹, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, —NR⁴¹C(O)(C1-C30 alkyl)-L-Z, —NR⁴¹C(O)(C1-C30 alkyl)-L-(C1-C30 alkyl)-Z, and —NR⁴¹C(O)-L-(C1-C30 alkyl)-Z; wherein L is a linker; wherein Z is selected from an antibody, an antibody fragment, a vitamin, a hormone, a carbohydrate, a molecular ligand, an aptamer, a non-antibody protein, a peptide, a nucleic acid, a fluorophore, and a drug; wherein each occurrence of R⁴¹ and R⁴², when present, is independently selected from hydrogen and C1-C12 alkyl; wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴; wherein each occurrence of Ar⁴, when present, is a structure represented by a formula:

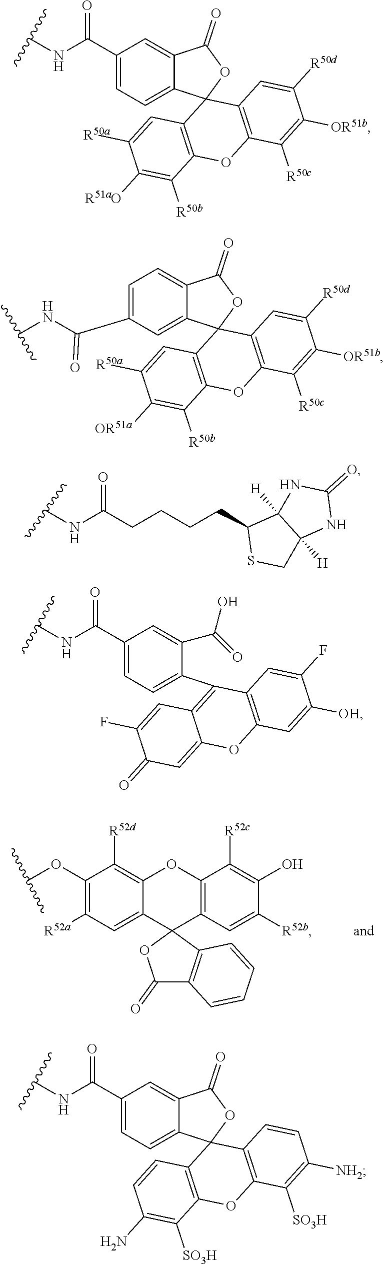

wherein each of R^{50a}, R^{50b}, R^{50c}, and R^{50d}, when present, is independently selected from hydrogen, —F, and —Cl; wherein each of R^{51a} and R^{51b}, when present, is independently selected from hydrogen and —C(O)(C1-C12 alkyl); wherein each of R^{52a}, R^{52b}, R^{52c}, and R^{52d}, when present, is independently selected from hydrogen, —F, and —Cl; wherein each occurrence of Ar³, when present, is a structure represented by a formula selected from:

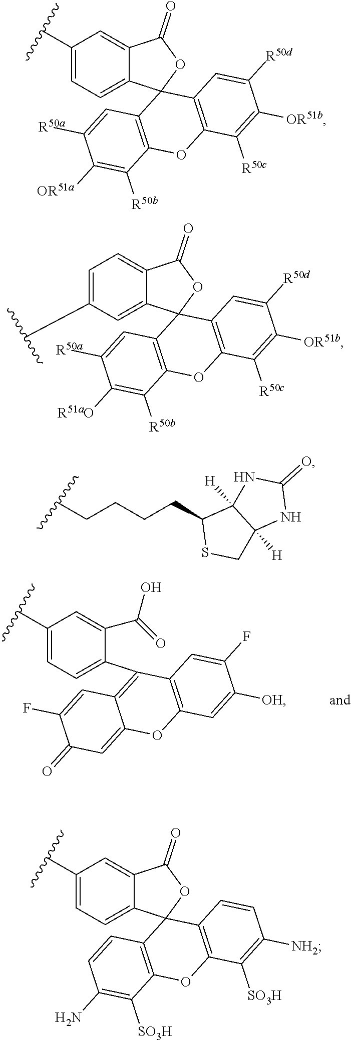

or wherein one of R⁸ and R^{8′} is absent; wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl); wherein each of R¹¹ and R¹² is independently selected from hydrogen, —OH, C1-C8 hydroxy, C1-C6 alkyl, C1-C8 alkoxy, and —OC(O)(C1-C8 alkyl); wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide); wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R²¹ is selected from hydrogen and C1-C6 alkyl; wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide); wherein Ar¹, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each of R²⁶ and R^{26′} is independently selected from hydrogen, —OH, C1-C8 hydroxy, and C1-C8 alkoxy; or wherein each of R²⁶ and R^{26′} together comprise ═O; wherein R²⁷ is selected from hydrogen and C1-C6 alkyl; wherein each of R²⁸ and R²⁹ is independently selected from hydrogen and halogen; or wherein each of R²⁸ and R²⁹ together comprise —O— or —N(R³⁷)—; wherein R³⁷, when present, is selected from hydrogen, C1-C4 alkyl, —SO₂R⁷¹, and a structure having a formula:

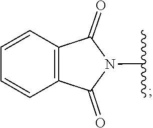 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that one and only one of R⁸ and R^{8′} is —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl)-L-Z, —NR⁴¹C(O)(C1-C30 alkyl)-L-(C1-C30 alkyl)-Z, or —NR⁴¹C(O)-L-(C1-C30 alkyl)-Z, and provided that when one or both of R²⁸ and R²⁹ is hydrogen then the occurrence of - - - - - - at C-22/C-23 is a double covalent bond, or a pharmaceutically acceptable salt thereof, the method comprising reacting a taccalonolide compound having a structure represented by a formula:

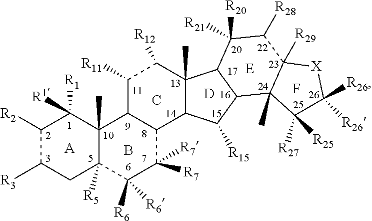

wherein each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr¹, —OC(O)(C1-C30 alkyl), —OC(O)Ar2, —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)R⁴⁰, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰; and wherein R⁴⁰ is a C1-C30 alkyl functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide, provided that one and only one of R⁶ and R^{6′} is —OC(O)R⁴⁰ or —NR⁴¹C(O)R⁴⁰, with a nucleophile having a structure represented by a formula selected from:

H—(C1-C30 alkyl)-L-Z, H-L-(C1-C30 alkyl)-Z, and H—(C1-C30 alkyl)-L-(C1-C30 alkyl)-Z.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF THE DRAWINGS

The accompanying figures, which are incorporated in and constitute a part of this specification, illustrate several aspects and together with the description serve to explain the principles of the invention.

FIG. 1 shows a representative schematic illustrating conjugation of a payload or headgroup (e.g., ADC, fluorescein, headgroup) to a tacca analog.

FIG. 2A-E show representative images illustrating compound no. 158F (FIG. 2A) in cells. Specifically, images illustrating the cellular localization of 10 μM compound after 24 hours in fixed cells where fluorescent tacca was co-localized with microtubules stained by immunofluorescence to β-tubulin (FIG. 2B and FIG. 2C), and after 8 hours in live HCC1937 cells (FIG. 2D). FIG. 2E shows a representative image of a 50 kDa protein (tubulin bound to compound no. 43DL158F) detected by immunoblotting with anti-fluorescein antibody after treatment of HCC1937 cells with 2.5 μM compound or vehicle for 4 hours.

FIG. 3 shows representative data illustrating the results of compound no. 154B in a biochemical purified tubulin polymerization assay as compared to tacca AJ.

FIG. 4A shows a representative image illustrating 10 μM compound no. 155G in HCC1806 cells after 24 hours.

FIG. 4B shows representative data illustrating the results of compound no. 155G in a biochemical purified tubulin polymerization assay.

FIG. 5A shows representative images illustrating 10 μM of compound no. 155F in HCC1806 cells after 24 hours.

FIG. 5B shows representative data illustrating the results of compound no. 155F in a purified biochemical tubulin polymerization assay.

FIG. 6 shows a representative image illustrating 5 μM compound no. 163D in HCC1937 cells after 6 hours in HBSS+Ca+Mg.

FIG. 7 shows a representative image illustrating 10 μM compound no. 164E in HCC1937 cells after 6 hours.

FIG. 8 shows representative images illustrating vehicle (left panel), 39 nM compound no. 164B (center panel), and 78 nM of compound no. 164B in HCC1937 cells after 6 hours.

FIG. 9 shows representative images of vehicle (ethanol), 5 μM of compound nos. 154J, 162C, 163B, 163D, and 163E, PTX-OG, and DMSO in HCC1937 cells with or without 10% pluronic F-127 after 6 hours in HBSS+Ca+Mg, imaged before washing. PTX-OG is commercial fluorescent paclitaxel live imaging reagent (ThermoFisher T34075) that requires the addition of pluronic F-127 and removal of excess dye from the medium for optimal imaging.

FIG. 10 shows representative images of vehicle (ethanol), 5 μM of compound nos. 154J, 162C, 163B, 163D, and 163E, PTX-OG, and DMSO with or without 10% pluronic F-127 in HCC1937 cells after 6 hours in HBSS+Ca+Mg, imaged after washing in HBSS.

FIG. 11 shows representative images of compound nos. 164B or the commercial taxane microtubule probe PTX-OG (ThermoFisher T34075) in SK-OV-3 cells (0.5 μM for 5 hours) at 37° C. or after an additional 20 min of chilling at −20° C., which depolymerizes taxane-stabilized, but not tacca-stabilized microtubules and results in loss of cellular staining for taxane, but not tacca probes. The same frame is shown in at both temperatures for internal reference.

FIG. 12 shows a representative image of compound nos. 164B or the commercial taxane microtubule probe siR-tubulin (Cytoskeleton/Spirochrome CY-SC002) in SK-OV-3 or isogenic P-glycoprotein (Pgp) expressing SK-OV-3-MDR-1-6/6 (SK-OV-3 M6/6) cells after treatment of cells for 5 hours with 0.5 μM in HBSS+Ca+Mg demonstrating that tacca probes, but not taxane probes retain staining in cells with high levels of drug efflux pump expression, even without the addition of Pgp inhibitors like verapamil that have physiological consequences in addition to their Pgp-inhibitory effects.

FIG. 13 shows a representative schematic illustrating synthetic routes to access antibody-drug conjugates (ADCs).

FIG. 14 shows representative images (a)-(e) showing that taccalonolide microtubule stabilizers covalently bind β-tubulin.

FIG. 15 shows representative structures of the semi-synthetic taccalonolide-based fluorescent probes 3-12.

FIG. 16 shows a representative synthetic protocol of Flu-tacca-7 (11).

FIG. 17A-E show representative data illustrating the optimization of taccalonolide-fluorescein probes.

FIG. 18A-H show representative data illustrating that the fluorescein moieties of the taccalonolide probes engage additional β-tubulin contacts and enhance the polymerization of purified tubulin.

FIG. 19 shows representative data illustrating the hydrolytic stability of taccalonolide-based probes.

FIG. 20 shows representative data illustrating concentration-response curves for the growth of cancer cells treated with taccalonolide or taccalonolide probes.

FIG. 21A and FIG. 21B show representative data illustrating the quantification of fluorescent intensity of taccalonolide probes.

FIG. 22 shows representative data illustrating the time course of microtubule polymerization and the binding of taccalonolide probe 12 to purified tubulin.

FIG. 23A shows representative data illustrating the cellular hydrolysis and deprotection of 10.

FIG. 23B shows representative data illustrating the structures of 10 and its deprotected analogue 28.

FIG. 24A-E show representative data illustrating that the 22,23-epoxy moiety of taccalonolides and D226 residue on β-tubulin are critical for the localization and binding of taccalonolides to β-tubulin.

FIG. 25A-D show representative data illustrating that the taccalonolide C-22 epoxide is critical for the localization and binding of taccalonolides to β-tubulin. HCC1937 cells were treated with 5 μM taccalonolide probes with (8) or without (7) the 22,23-epoxide for 6 h.

FIG. 26A-E show representative data illustrating the systematic evaluation of critical β-tubulin residues that mediate the binding affinity of taccalonolides to β-tubulin.

FIG. 27A and FIG. 27B show representative data illustrating the incorporation of GFP-tagged β-tubulin mutants into microtubules. GFP-tagged mutants were visualized in live HeLa cells before (FIG. 27A) and after (FIG. 27B) treatment with 100 nM 2 for 22 h.

FIG. 28A-D show representative data illustrating non-covalent interactions between selected β-tubulin residues and 12.

FIG. 29A and FIG. 29B show representative data illustrating the time course of 11 binding to wild type (WT) or mutant β-tubulin. HeLa cells were transfected with GFP-tagged TUBB1 constructs with indicated mutations then treated with 1 μM 11 for 1-8 h. Probe-treated cells were lysed and subjected to immunoblotting for fluorescein or β-tubulin. Ratio of 11 bound to endogenously expressed tubulin (FIG. 29A) or ectopically expressed tubulin constructs (FIG. 29B), mutant or WT, was normalized to the ratio of the WT form bound at 8 h. Average±SEM for n=2 independent experiments. One-way ANOVA and Tukey's post-hoc test were used to calculate statistical significance between each condition and significant differences between the binding of 11 to the mutants at each time point as compared to wild type control are depicted:*p<0.05, **p<0.01, ***p<0.001.

FIG. 30A and FIG. 30B show representative data illustrating that taccalonolide probes are superior for imaging studies compared to commercial taxane-based microtubule probes.

FIG. 31A and FIG. 31B show representative data illustrating the effect of pluronic F-127 on probe imaging.

FIG. 32 shows representative data illustrating P-glycoprotein (Pgp) expression.

FIG. 33A-C show representative data illustrating that taccalonolide probes retain potency and efficacy in βIII-tubulin expressing cells.

FIG. 34A and FIG. 34B show representative data illustrating that the effect of pluronic F-127 on probe imaging. Specifically, FIG. 34A shows the comparison of Tubulin tracker green in SK-OV-3 or SK-OV-3-MDR-1-M6/6 cells with or without pluronic F-127 which facilitates probe loading and reduces background signal. FIG. 34B shows images of chilled SK-OV-3 cells from FIG. 30A and FIG. 30B were hyper-contrasted to visualize low signal intensity. Scale bars=50 μm.

FIG. 35A-C shows representative data illustrating that taccalonolide probes retain potency and efficacy in βIII-tubulin expressing cells. Specifically, FIG. 35A shows the expression of total β-tubulin and the βIII-isotype of tubulin in HeLa cells and an isogenic line that overexpresses this isotype (βIII-HeLa). FIG. 35B shows that the taccalonolide probe 11 retains antiproliferative and cytotoxic potency and efficacy in the βIII-tubulin expressing cell line (open circle) as compared to the parental HeLa cell line (closed circle). Each point represents mean±SEM from n=4 biologically independent experiments for βIII-HeLa cells and n=3 independent experiments for HeLa cells. FIG. 35C shows that the taccalonolide probe 11 retains the ability to bind cellular microtubules in the βIII-tubulin expressing cell line as compared to the parental HeLa cell line. Cells were treated with 0.5 μM 11 for 5 h and imaged under identical acquisition conditions.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

Listed below are definitions of various terms used to describe this invention. These definitions apply to the terms as they are used throughout this specification, unless otherwise limited in specific instances, either individually or as part of a larger group.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” mean that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. In one aspect, the subject is a mammal. A patient refers to a subject afflicted with a viral infection. The term “patient” includes human and veterinary subjects. In some aspects of the disclosed methods, the subject has been diagnosed with a need for treatment of one or more viral infections prior to the administering step. In various aspects, the one or more disorders is selected from chikungunya, Venezuelan equine encephalitis, dengue, influenza, and zika.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. In some aspects of the disclosed methods, the subject has been diagnosed with a need for treatment of a viral infection prior to the administering step. As used herein, the phrase “identified to be in need of treatment for a disorder,” or the like, refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “treating” refers to relieving the disease, disorder, or condition, i.e., causing regression of the disease, disorder, and/or condition. The term “preventing” refers to preventing a disease, disorder, or condition from occurring in a human or an animal that may be predisposed to the disease, disorder and/or condition, but has not yet been diagnosed as having it; and/or inhibiting the disease, disorder, or condition, i.e., arresting its development.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein.

The term “comprising” (and its grammatical variations) as used herein is used in the inclusive sense of “having” or “including” and not in the exclusive sense of “consisting of.”

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

“Pharmaceutically acceptable salts” refer to derivatives of the disclosed compounds wherein the parent compound is modified by making acid or base salts thereof. The compounds of this disclosure form acid addition salts with a wide variety of organic and inorganic acids and include the physiologically acceptable salts which are often used in pharmaceutical chemistry. Such salts are also part of this disclosure. Typical inorganic acids used to form such salts include hydrochloric, hydrobromic, hydroiodic, nitric, sulfuric, phosphoric, hypophosphoric acid, and the like. Salts derived from organic acids, such as aliphatic mono- and dicarboxylic acids, phenyl substituted alkanoic acids, hydroxyalkanoic and hydroxyalkandioic acids, aromatic acids, aliphatic and aromatic sulfonic acids may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, ρ-hydroxybutyrate, butyne-1,4-dioate, hexyne-1,4-dioate, caprate, caprylate, chloride, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzene-sulfonate, p-bromobenzenesulfonate, chlorobenzenesulfonate, ethanesulfonate, 2-hydroxyethanesulfonate, methanesulfonate, naphthalene-1-sulfonate, naphthalene-2-sulfonate, p-toleunesulfonate, xylenesulfonate, tartarate, and the like.

It is understood that the compounds of the present disclosure relate to all optical isomers and stereo-isomers at the various possible atoms of the molecule, unless specified otherwise. Compounds may be separated or prepared as their pure enantiomers or diastereomers by crystallization, chromatography or synthesis.

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkyl alcohol” is used in another, it is not meant to imply that the term “alkyl” does not also refer to specific terms such as “alkyl alcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes pyridinde, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “hydroxyl” as used herein is represented by the formula —OH.

The term “ketone” as used herein is represented by the formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by the formula A'S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C1-4 straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N3, —(CH2)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C1-4 straight or branched alkylene)C(O)OR^{●}, or —SSR^{●} wherein each R^{●} is unsubstituted or where preceded by “halo” is substituted only with one or more halogens, and is independently selected from C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

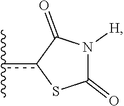

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non- isotopically labeled reagent.

The compounds described in the invention can be present as a solvate. “Solvates” refers to the compound formed by the interaction of a solvent and a solute and includes hydrates. Solvates are usually crystalline solid adducts containing solvent molecules within the crystal structure, in either stoichiometric or nonstoichiometric proportions. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

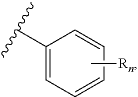

which is understood to be equivalent to a formula:

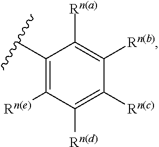

wherein n is typically an integer. That is, Rⁿ is understood to represent five independent substituents, R^{n(a)}, R^{n(b)}, R^{n(c)}, R^{n(d)}, R^{n(e)}. In each such case, each of the five Rⁿ can be hydrogen or a recited substituent. By “independent substituents,” it is meant that each R substituent can be independently defined. For example, if in one instance R^{n(a)} is halogen, then R^{n(b)} is not necessarily halogen in that instance.

In some yet further aspects, a structure of a compound can be represented by a formula:

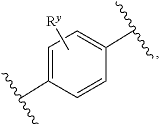

wherein RY represents, for example, 0-2 independent substituents selected from A¹, A², and A³, which is understood to be equivalent to the groups of formulae:
wherein RY represents 0 independent substituents

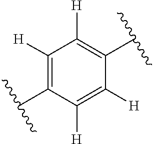 [SMILES: *c1ccc(*)cc1]

wherein RY represents 1 independent substituent

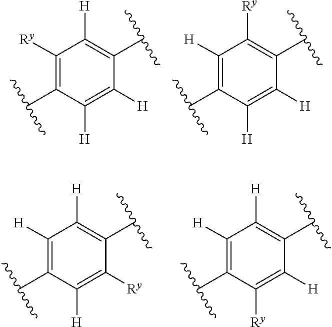

wherein RY represents 2 independent substituents

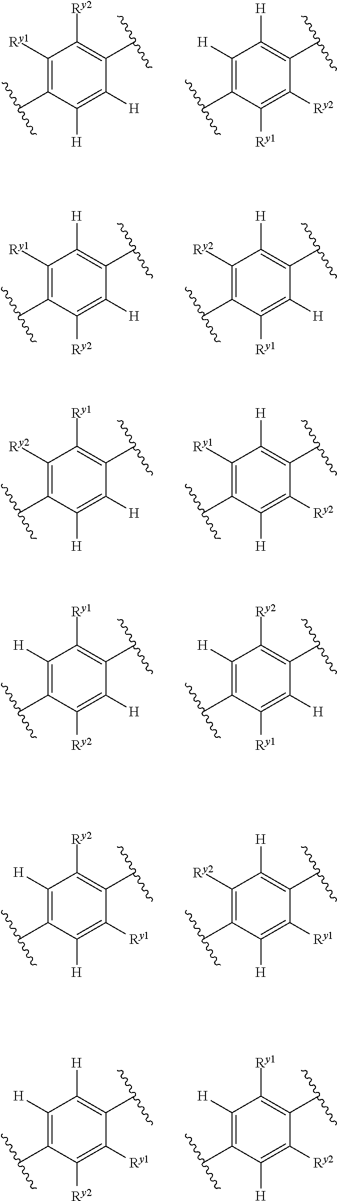

Again, by “independent substituents,” it is meant that each R substituent can be independently defined. For example, if in one instance R^{y1} is A¹, then R^{y2} is not necessarily A¹ in that instance.

In some further aspects, a structure of a compound can be represented by a formula,

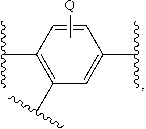

wherein, for example, Q comprises three substituents independently selected from hydrogen and A, which is understood to be equivalent to a formula:

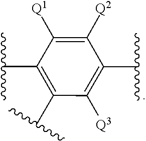

Again, by “independent substituents,” it is meant that each Q substituent is independently defined as hydrogen or A, which is understood to be equivalent to the groups of formulae:

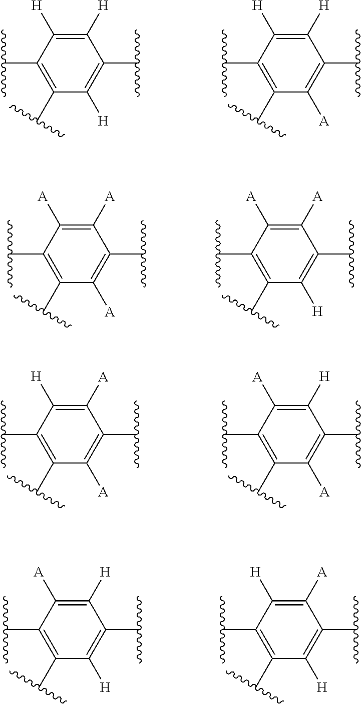

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, the invention relates to taccalonolide microtubule stabilizers useful as cellular probes (e.g., for the detection, visualization, and/or quantification of a target). The disclosed compounds are also useful in the treatment of hyperproliferative disorders including, but not limited to, cardiovascular diseases such as, for example, coronary heart disease, stroke, hypertensive heart disease, inflammatory heart disease, and rheumatic heart disease, and cancers such as, for example, a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma).

In one aspect, the compounds of the invention are useful as a cellular probe such as, for example, a tubulin-labeling probe.

In one aspect, the compounds of the invention are useful as ADCs.

In one aspect, the compounds of the invention are useful in the treatment of cancers, as further described herein.

In one aspect, the compounds of the invention are useful in the treatment of cardiovascular disease, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Structure

In one aspect, disclosed are compounds having a structure represented by a formula:

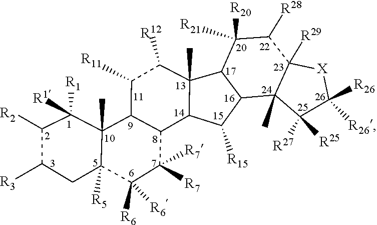

wherein each occurrence of - - - - - - is a single or double covalent bond; wherein X is selected from O, NR^{x}, and C(R^{x})₂; wherein each occurrence of R^{x}, when present, is independently selected from hydrogen and C1-C6 alkyl; wherein R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹; wherein each occurrence of R³¹, R³², R³⁴, R^{35a}, and R^{35b}, when present, is independently selected from hydrogen and C1-C12 alkyl; wherein each occurrence of R³³, when present, is independently selected from hydrogen, C1-C12 alkyl, and monocyclic aryl monosubstituted with a methyl group; wherein each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and wherein R^{1′} is hydrogen; or wherein each of R¹ and R^{1′} together comprise ═O or ═NR³⁶; wherein each occurrence of R³⁶, when present, is independently selected from hydrogen and C1-C12 alkyl; wherein each of R² and R³ is independently selected from hydrogen, —OH, C1-C12 hydroxy, and halogen; or wherein each of R₂ and R₃ together comprise —O—; wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; wherein each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar1, —OAr1, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)R⁴⁰, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰; wherein each occurrence of R⁴⁰, when present, is independently a C1-C30 alkyl functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide; wherein each occurrence of R⁴¹ and R⁴², when present, is independently selected from hydrogen and C1-C12 alkyl; wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴; wherein each occurrence of Ar⁴, when present, is a structure represented by a formula selected from:

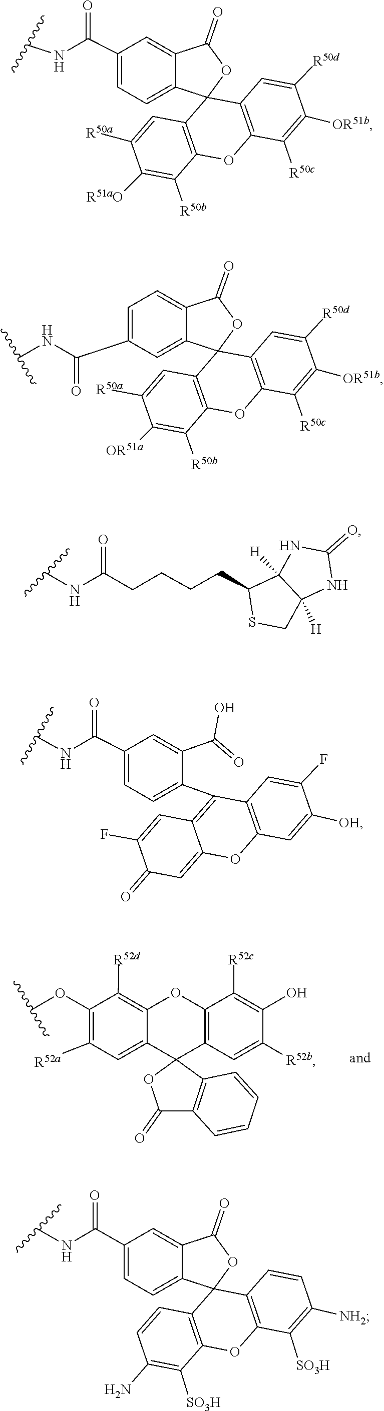

wherein each of R^{50a}, R^{50b}, R^{50c}, and R^{50d}, when present, is independently selected from hydrogen, —F, and —Cl; wherein each of R^{51a} and R^{51b}, when present, is independently selected from hydrogen and —C(O)(C1-C12 alkyl); wherein each of R^{52a}, R^{52b}, R^{52c}, and R^{52d}, when present, is independently selected from hydrogen, —F, and —Cl; wherein each occurrence of Ar³, when present, is a structure represented by a formula selected from:

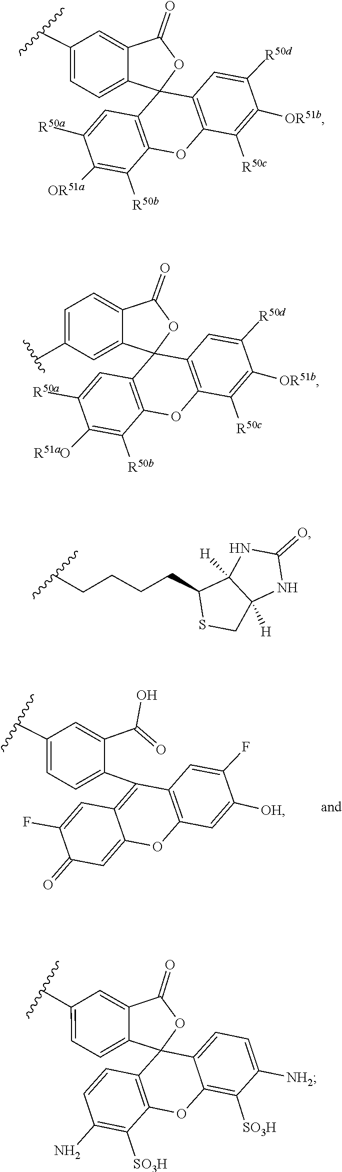

or wherein one of R⁶ and R^{6′} is absent; wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl); or wherein each of R⁷ and R^{7′} together comprise ═O; or wherein one of R⁷ and R^{7′} is absent; wherein each of R¹¹ and R¹² is independently selected from hydrogen, —OH, C1-C8 hydroxy, C1-C6 alkyl, C1-C8 alkoxy, and —OC(O)(C1-C8 alkyl); wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide); wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R²¹ is selected from hydrogen and C1-C6 alkyl; wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide); wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each of R²⁶ and R^{26′} is independently selected from hydrogen, —OH, C1-C8 hydroxy, and C1-C8 alkoxy; or wherein each of R²⁶ and R^{26′} together comprise ═O; wherein R²⁷ is selected from hydrogen and C1-C6 alkyl; wherein each of R²⁸ and R²⁹ is independently selected from hydrogen and halogen; or wherein each of R²⁸ and R²⁹ together comprise —O— or —N(R³⁷)—; wherein R³⁷, when present, is selected from hydrogen, C1-C4 alkyl, —SO₂R⁷¹, and a structure having a formula:

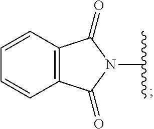 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that at least one of R⁶ and R^{6′} is —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, or —NR⁴¹C(O)R⁴⁰, and provided that when one or both of R²⁸ and R²⁹ is hydrogen then the occurrence of - - - - - - at C-22/C-23 is a double covalent bond, or a pharmaceutically acceptable salt thereof.

In one aspect, disclosed are compounds having a structure represented by a formula:

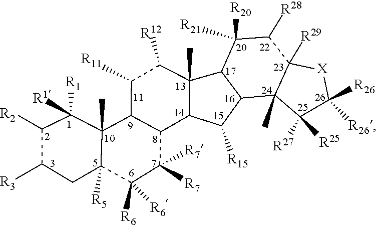

wherein each occurrence of - - - - - - is a single or double covalent bond; wherein X is selected from O, NR^{x} and C(R^{x})₂; wherein each occurrence of R^{x}, when present, is independently selected from hydrogen and C1-C6 alkyl; wherein R¹ is selected from hydrogen, halogen, —OH, —CN, —NC, —NCO, —OCN, —NO₂, —ONO₂, —ONO, —NO, —N₃, —NH₂, —NH₃, —N═NR³, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar1, (C1-C12 alkyl)Ar1, and —OAr¹; wherein each occurrence of R³¹, R³², R³⁴, R^{35a}, and R^{35b}, when present, is independently selected from hydrogen and C1-C12 alkyl; wherein each occurrence of R³³, when present, is independently selected from hydrogen, C1-C12 alkyl, and monocyclic aryl monosubstituted with a methyl group; wherein each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and wherein R^{1′} is hydrogen; or wherein each of R¹ and R^{1′} together comprise ═O or ═NR³⁶; wherein each occurrence of R³⁶, when present, is independently selected from hydrogen and C1-C12 alkyl; wherein each of R² and R³ is independently selected from hydrogen, —OH, C1-C12 hydroxy, and halogen; or wherein each of R₂ and R₃ together comprise —O—; wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; wherein each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar1, —OAr1, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)R⁴⁰, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰; wherein each occurrence of R⁴⁰, when present, is independently a C1-C30 alkyl functionalized with a group selected from —SH, —NH₂, —NHOH, terminal alkyne, haloacetyl, maleimide, isothiocyanate, N-hydroxysuccinimde, succinimidyl ester, tetrafluorophenyl ester, sulfodichlorophenol ester, and hydrazide; wherein each occurrence of R⁴¹ and R⁴², when present, is independently selected from hydrogen and C1-C12 alkyl; wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴; wherein each occurrence of Ar⁴, when present, is a structure represented by a formula:

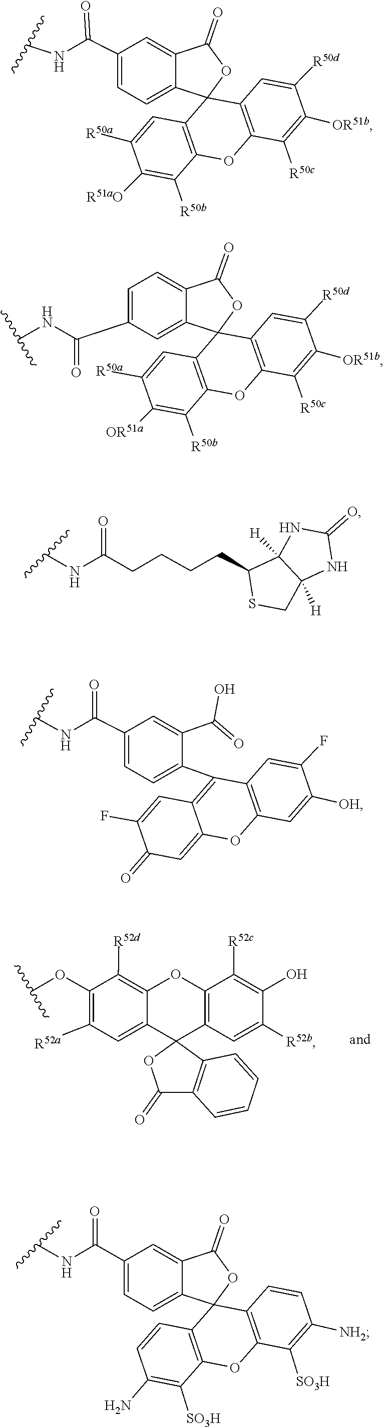

wherein each of R^{50a}, R^{50b}, R^{50c}, and R^{50d}, when present, is independently selected from hydrogen, —F, and —Cl; wherein each of R^{51a} and R^{51b}, when present, is independently selected from hydrogen and —C(O)(C1-C12 alkyl); wherein each of R^{52a}, R^{52b}, R^{52c}, and R^{52d}, when present, is independently selected from hydrogen, —F, and —Cl; wherein each occurrence of Ar³, when present, is a structure represented by a formula selected from:

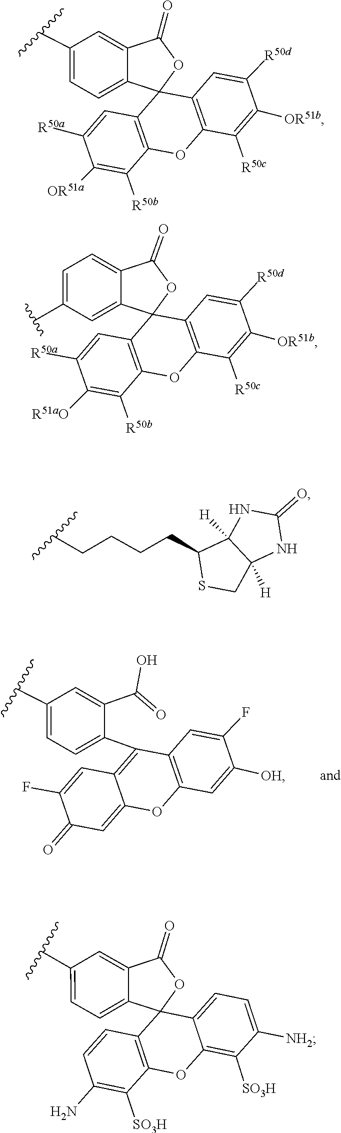

or wherein one of R⁶ and R^{6′} is absent; wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl); or wherein each of R⁷ and R^{7′} together comprise ═O; or wherein one of R⁷ and R^{7′} is absent; wherein each of R¹¹ and R¹² is independently selected from hydrogen, —OH, C1-C8 hydroxy, C1-C6 alkyl, C1-C8 alkoxy, and —OC(O)(C1-C8 alkyl); wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide); wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R²¹ is selected from hydrogen and C1-C6 alkyl; wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide); wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each of R²⁶ and R^{26′} is independently selected from hydrogen, —OH, C1-C8 hydroxy, and C1-C8 alkoxy; or wherein each of R²⁶ and R^{26′} together comprise ═O; wherein R²⁷ is selected from hydrogen and C1-C6 alkyl; wherein each of R²⁸ and R²⁹ is independently selected from hydrogen and halogen; or wherein each of R²⁸ and R²⁹ together comprise —O— or —N(R³⁷)—; wherein R³⁷, when present, is selected from hydrogen, C1-C4 alkyl, —SO₂R⁷¹, and a structure having a formula:

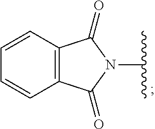 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that at least one of R⁶ and R^{6′} is —OC(O)R⁴⁰ or —NR⁴¹C(O)R⁴⁰, and provided that when one or both of R²⁸ and R²⁹ is hydrogen then the occurrence of - - - - - - at C-22/C-23 is a double covalent bond, or a pharmaceutically acceptable salt thereof.

In one aspect, disclosed are compounds having a structure represented by a formula:

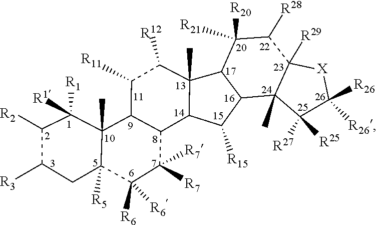

wherein each occurrence of - - - - - - is a single or double covalent bond; wherein X is selected from O, NR^{x} and C(R^{x})₂; wherein each occurrence of R^{x}, when present, is independently selected from hydrogen and C1-C6 alkyl; wherein R^{x} is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R_{35b}, —(C1-C12 alkyl)OC(O)NR^{35a}R^{35b}, Cy¹, Ar¹, (C1-C12 alkyl)Ar¹, and —OAr¹; wherein each occurrence of R³¹, R³², R³⁴, R^{35a}, and R^{35b}, when present, is independently selected from hydrogen and C1-C12 alkyl; wherein each occurrence of R³³, when present, is independently selected from hydrogen, C1-C12 alkyl, and monocyclic aryl monosubstituted with a methyl group; wherein each occurrence of Cy1, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and wherein R^{1′} is hydrogen; or wherein each of R¹ and R^{1′} together comprise ═O or ═NR³⁶; wherein each occurrence of R³⁶, when present, is independently selected from hydrogen and C1-C12 alkyl; wherein each of R² and R³ is independently selected from hydrogen, —OH, C1-C12 hydroxy, and halogen; or wherein each of R₂ and R₃ together comprise —O—; wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl); or wherein each of R⁷ and R^{7′} together comprise ═O; or wherein one of R⁷ and R^{7′} is absent; wherein each of R⁸ and R^{8′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr¹, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, —NR⁴¹C(O)(C1-C30 alkyl)-L-Z, —NR⁴¹C(O)(C1-C30 alkyl)-L-(C1-C30 alkyl)-Z, and —NR⁴¹C(O)-L-(C1-C30 alkyl)-Z; wherein L is a linker; wherein Z is selected from an antibody, an antibody fragment, a vitamin, a hormone, a carbohydrate, a molecular ligand, an aptamer, a non-antibody protein, a peptide, a nucleic acid, a fluorophore, and a drug; wherein each occurrence of R⁴¹ and R⁴², when present, is independently selected from hydrogen and C1-C12 alkyl; wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴; wherein each occurrence of Ar⁴, when present, is a structure represented by a formula:

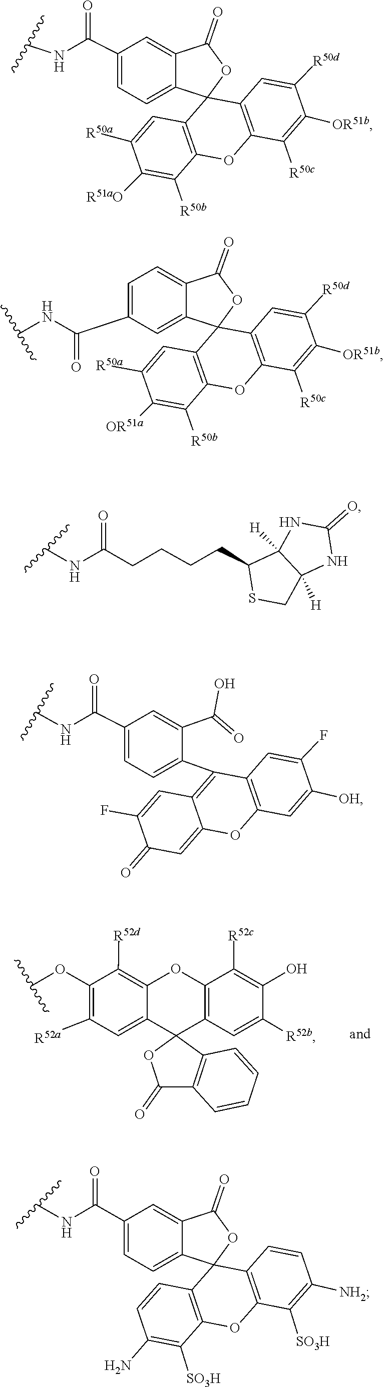

wherein each of R^{50a}, R^{50b}, R^{50c}, and R^{50d}, when present, is independently selected from hydrogen, —F, and —Cl; wherein each of R^{51a} and R^{51b}, when present, is independently selected from hydrogen and —C(O)(C1-C12 alkyl); wherein each of R^{52a}, R^{52b}, R^{52c}, and R^{52d}, when present, is independently selected from hydrogen, —F, and —Cl; wherein each occurrence of Ar³, when present, is a structure represented by a formula selected from:

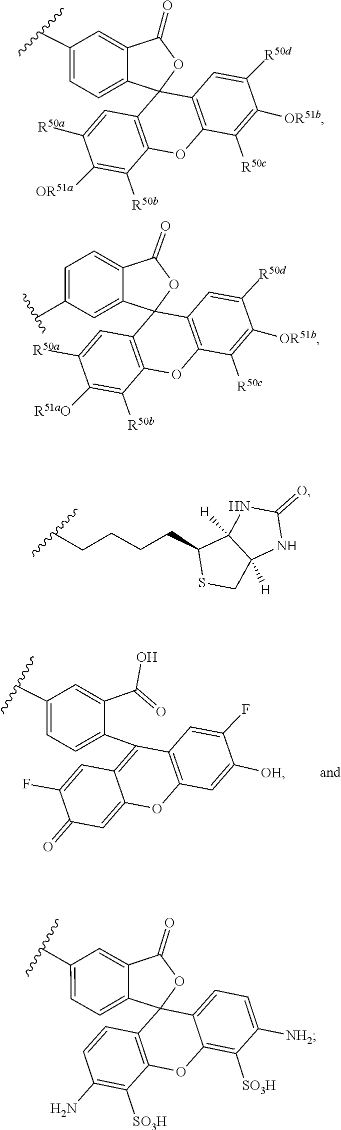

or wherein one of R⁸ and R^{8′} is absent; wherein each of R¹¹ and R¹² is independently selected from hydrogen, —OH, C1-C8 hydroxy, C1-C6 alkyl, C1-C8 alkoxy, and —OC(O)(C1-C8 alkyl); wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide); wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R²¹ is selected from hydrogen and C1-C6 alkyl; wherein R²¹ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide); wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each of R²⁶ and R^{26′} is independently selected from hydrogen, —OH, C1-C8 hydroxy, and C1-C8 alkoxy; or wherein each of R²⁶ and R^{26′} together comprise ═O; wherein R²⁷ is selected from hydrogen and C1-C6 alkyl; wherein each of R²⁸ and R²⁹ is independently selected from hydrogen and halogen; or wherein each of R²⁸ and R²⁹ together comprise —O— or N(R³⁷)—; wherein R³⁷, when present, is selected from hydrogen, C1-C4 alkyl, —SO₂R⁷¹, and a structure having a formula:

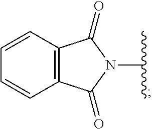 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that one and only one of R⁸ and R^{8′} is —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl)-L-Z, —NR⁴¹C(O)(C1-C30 alkyl)-L-(C1-C30 alkyl)-Z, or —NR⁴¹C(O)-L-(C1-C30 alkyl)-Z, and provided that when one or both of R²⁸ and R²⁹ is hydrogen then the occurrence of - - - - - - at C-22/C-23 is a double covalent bond, or a pharmaceutically acceptable salt thereof.

In one aspect, disclosed are compounds having a structure selected from:

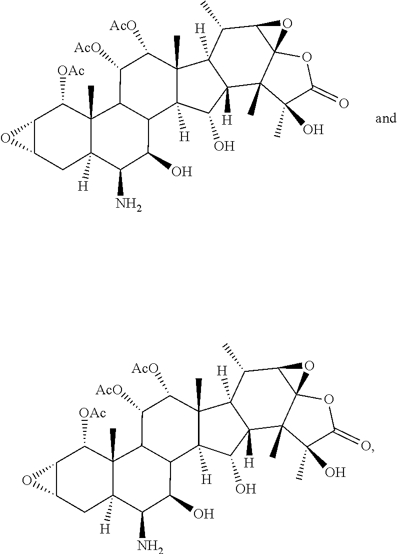 [SMILES: CC(=O)O[C@H]1C2C([C@@H](O)[C@@H](N)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@@H](N)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

or a pharmaceutically acceptable salt thereof.

In one aspect, disclosed are compounds selected from:

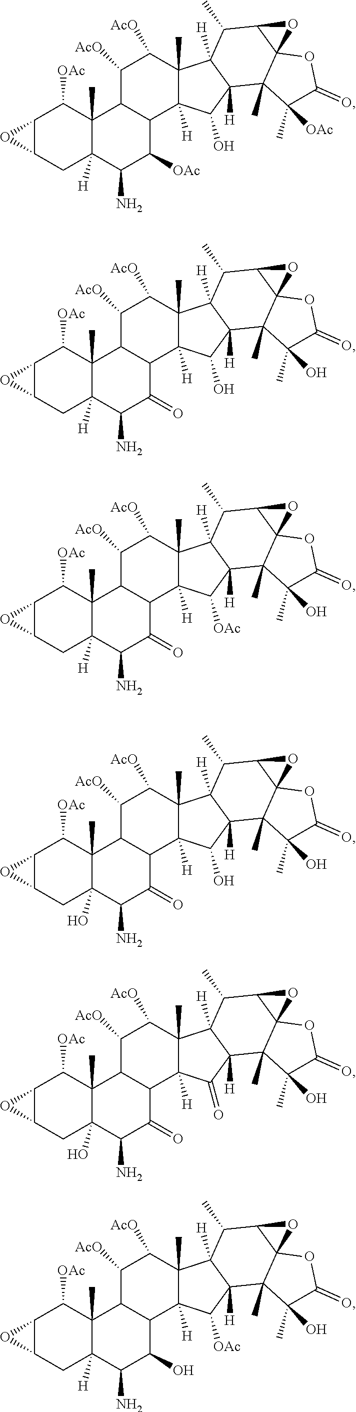

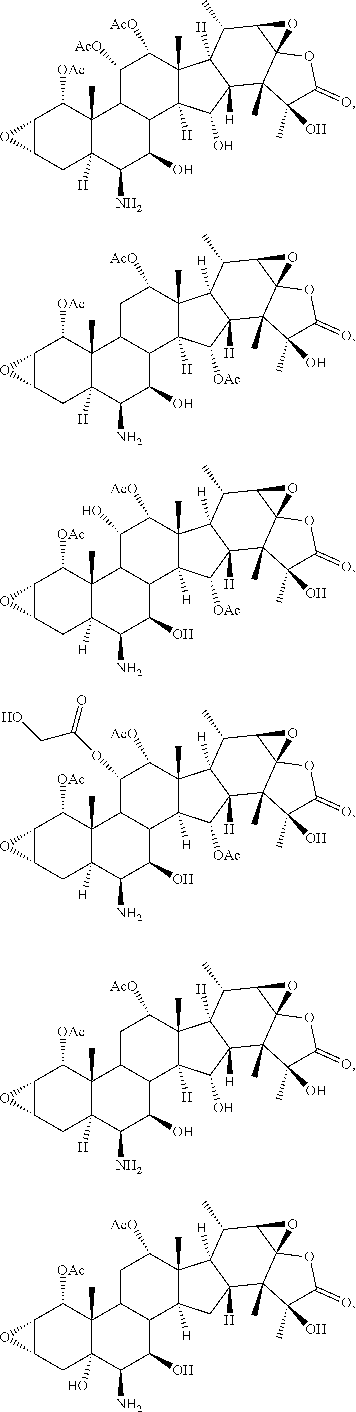

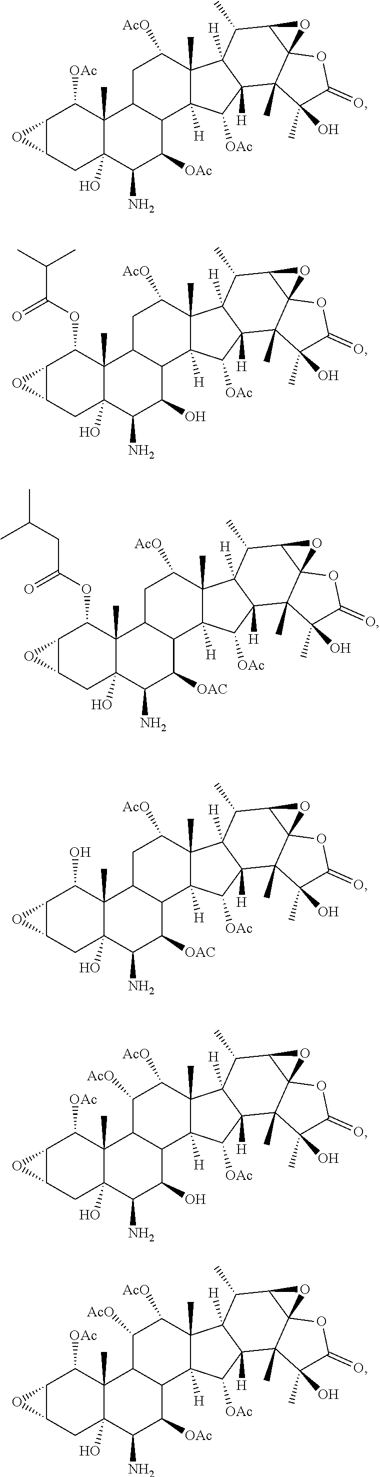

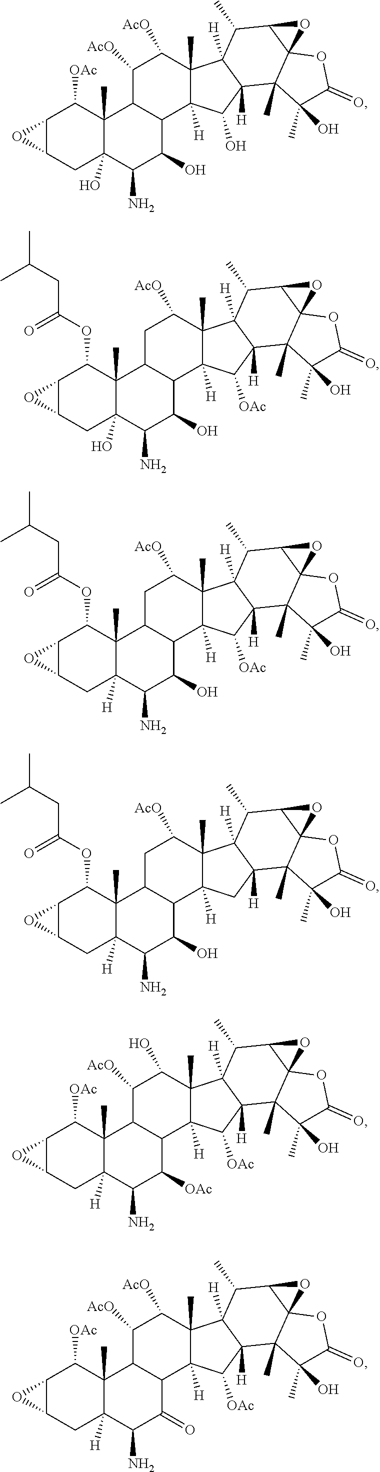

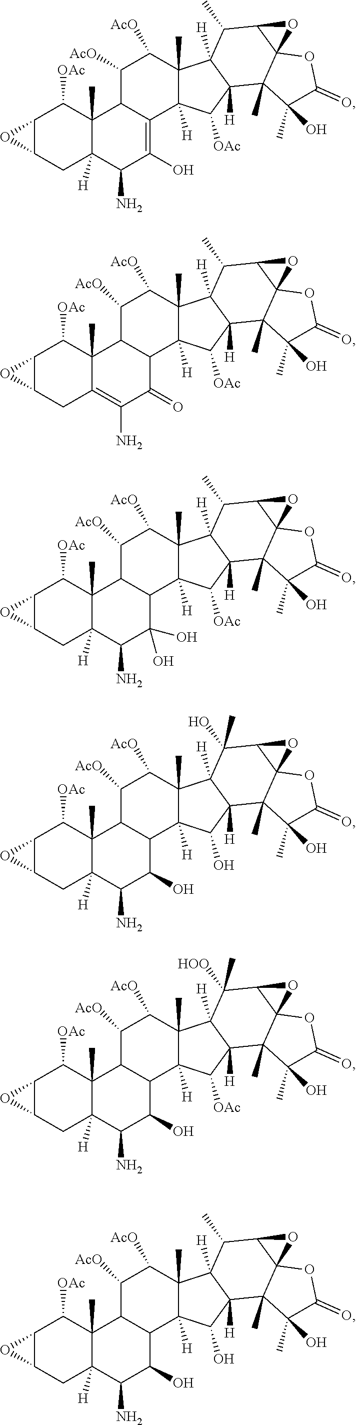

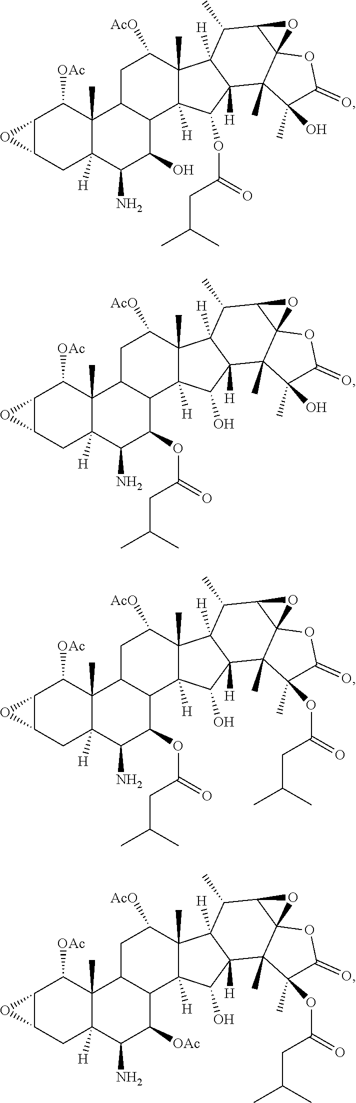

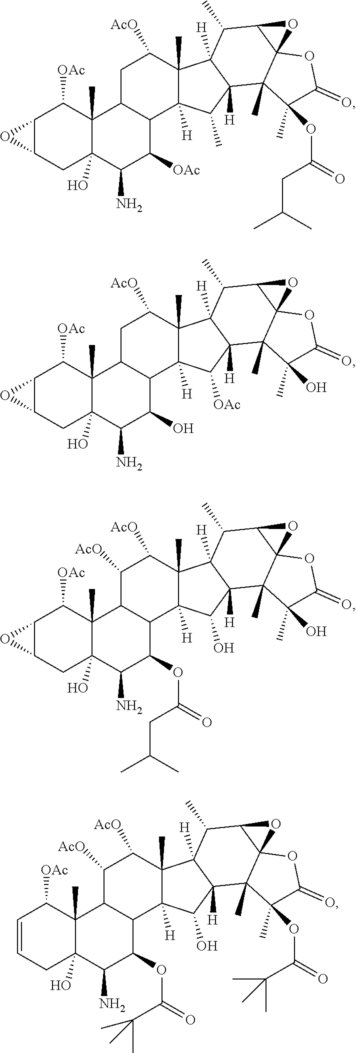

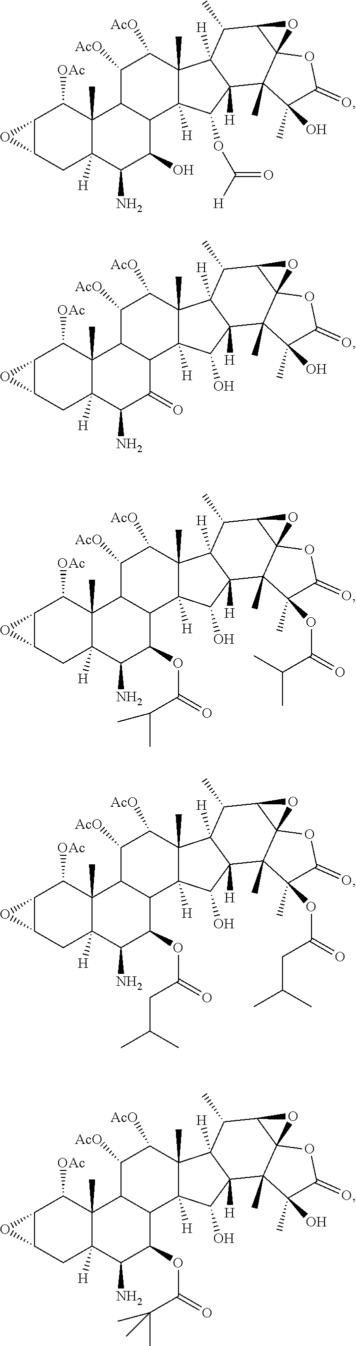

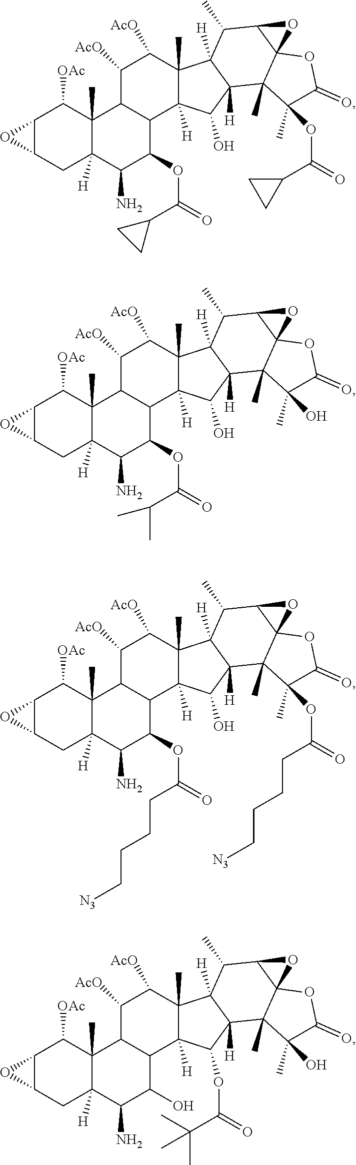

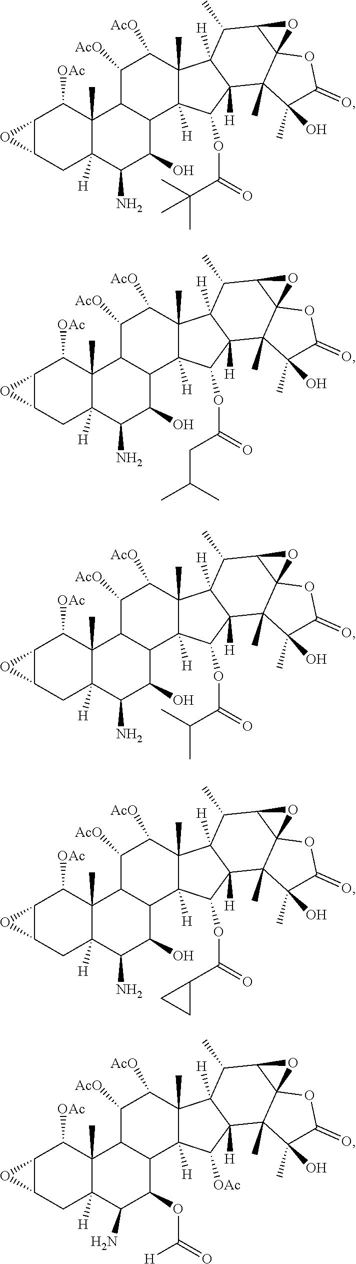

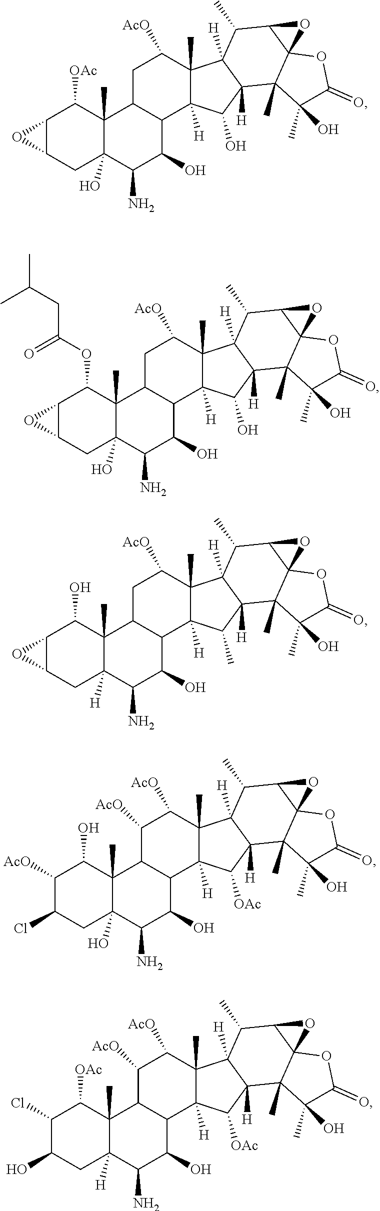

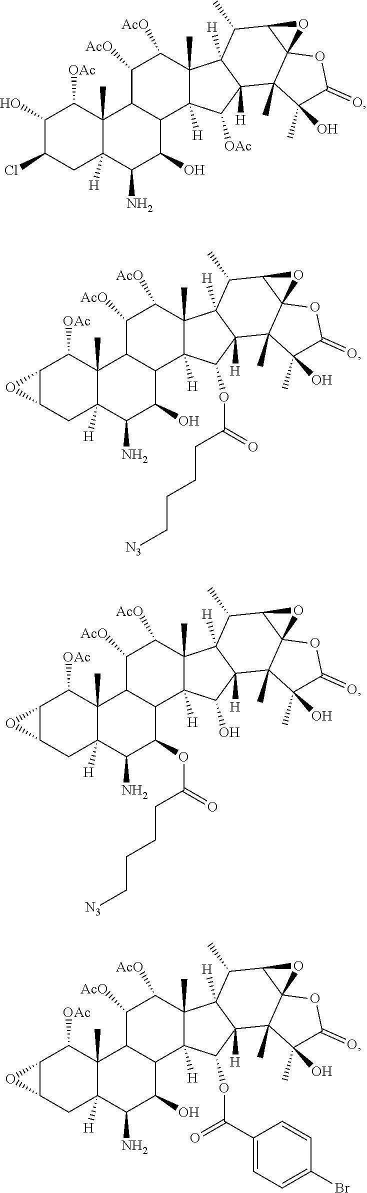

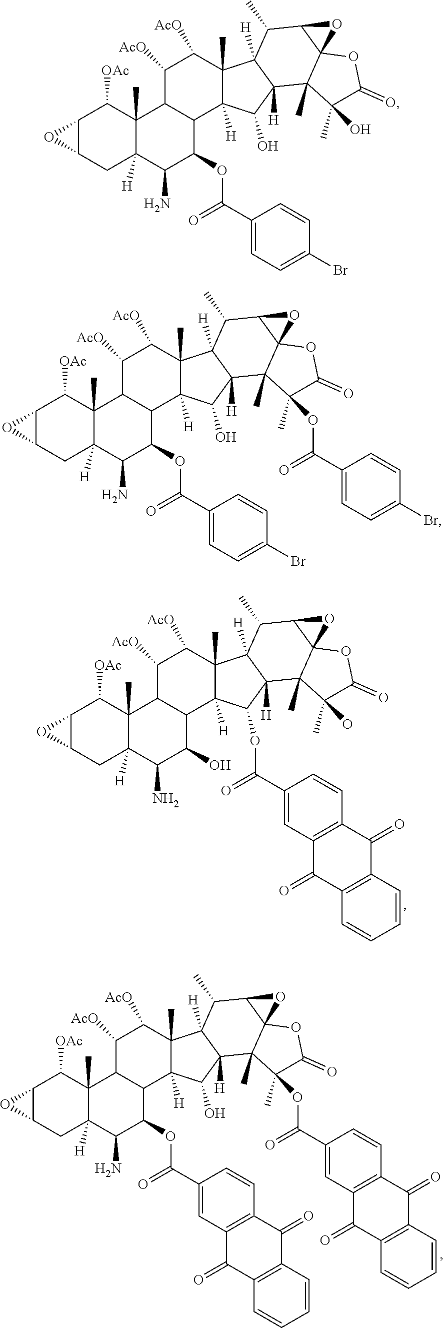

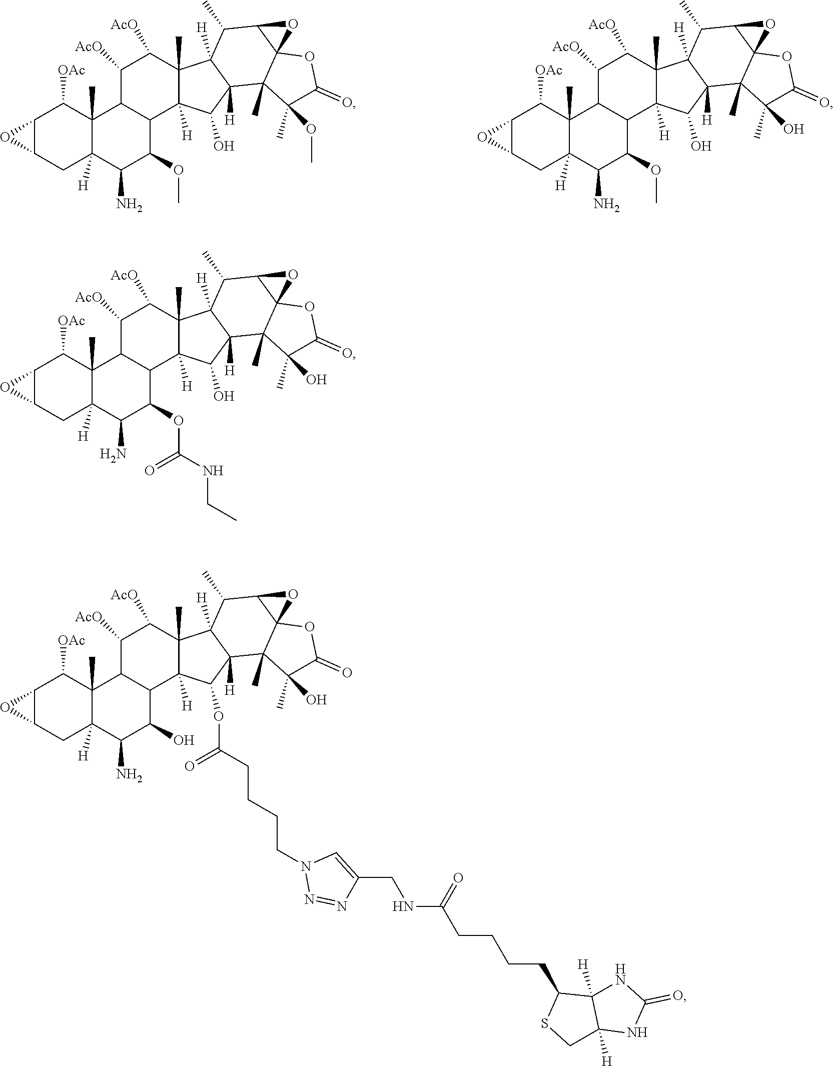

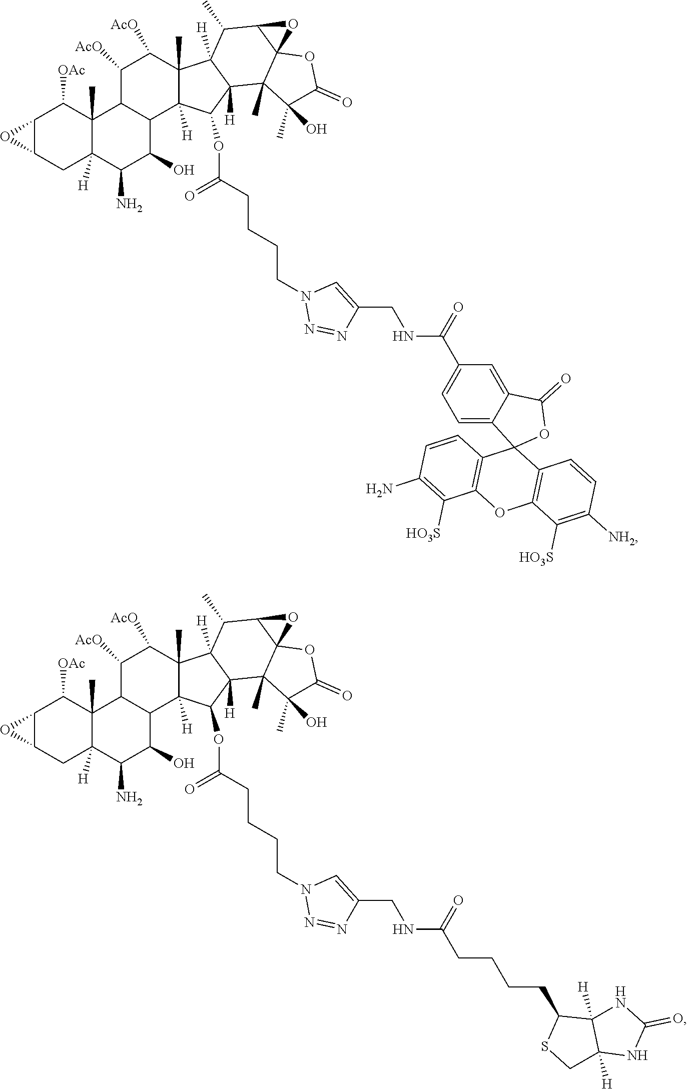

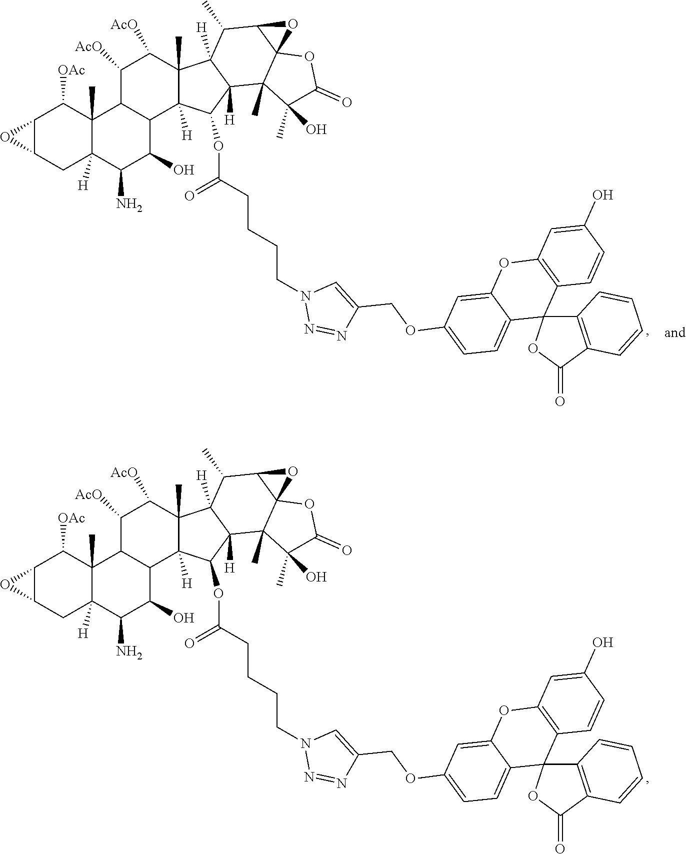 [SMILES: CC(=O)O[C@H]1C2C([C@@H](O)[C@@H](N)[C@H]3C[C@@H]4O[C@@H]4[C@H](OC(C)=O)[C@]23C)[C@@H]2[C@@H](OC(=O)CCCCn3cc(COc4ccc5c(c4)Oc4cc(O)ccc4C54OC(=O)c5ccccc54)nn3)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@@H](N)[C@H]3C[C@@H]4O[C@@H]4[C@H](OC(C)=O)[C@]23C)[C@@H]2[C@H](OC(=O)CCCCn3cc(COc4ccc5c(c4)Oc4cc(O)ccc4C54OC(=O)c5ccccc54)nn3)[C@@H]3[C@H]([C@H](C)[C@H]4O[C@]45OC(=O)[C@@](C)(O)[C@]35C)[C@@]2(C)[C@H]1OC(C)=O]

or a pharmaceutically acceptable salt thereof.

In one aspect, disclosed are compounds selected from:

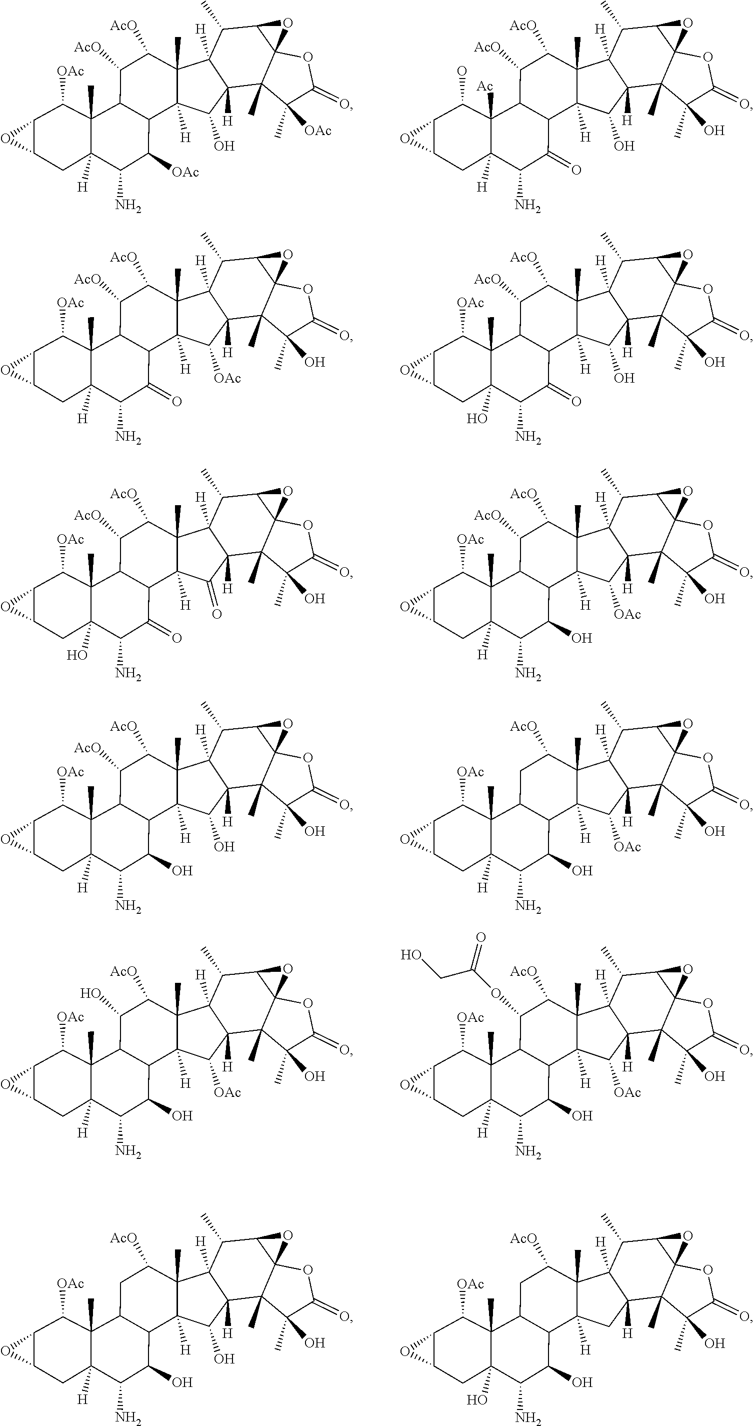

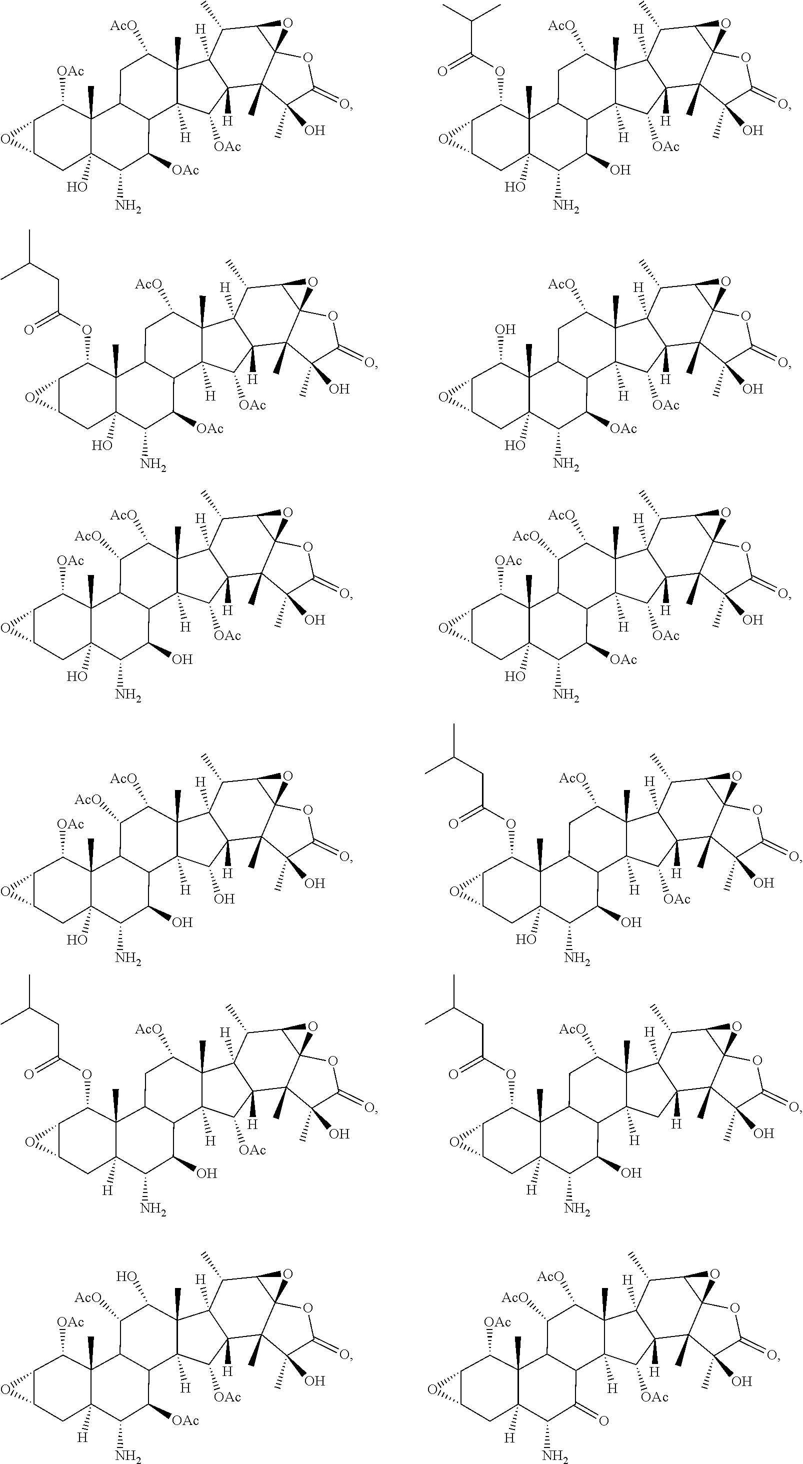

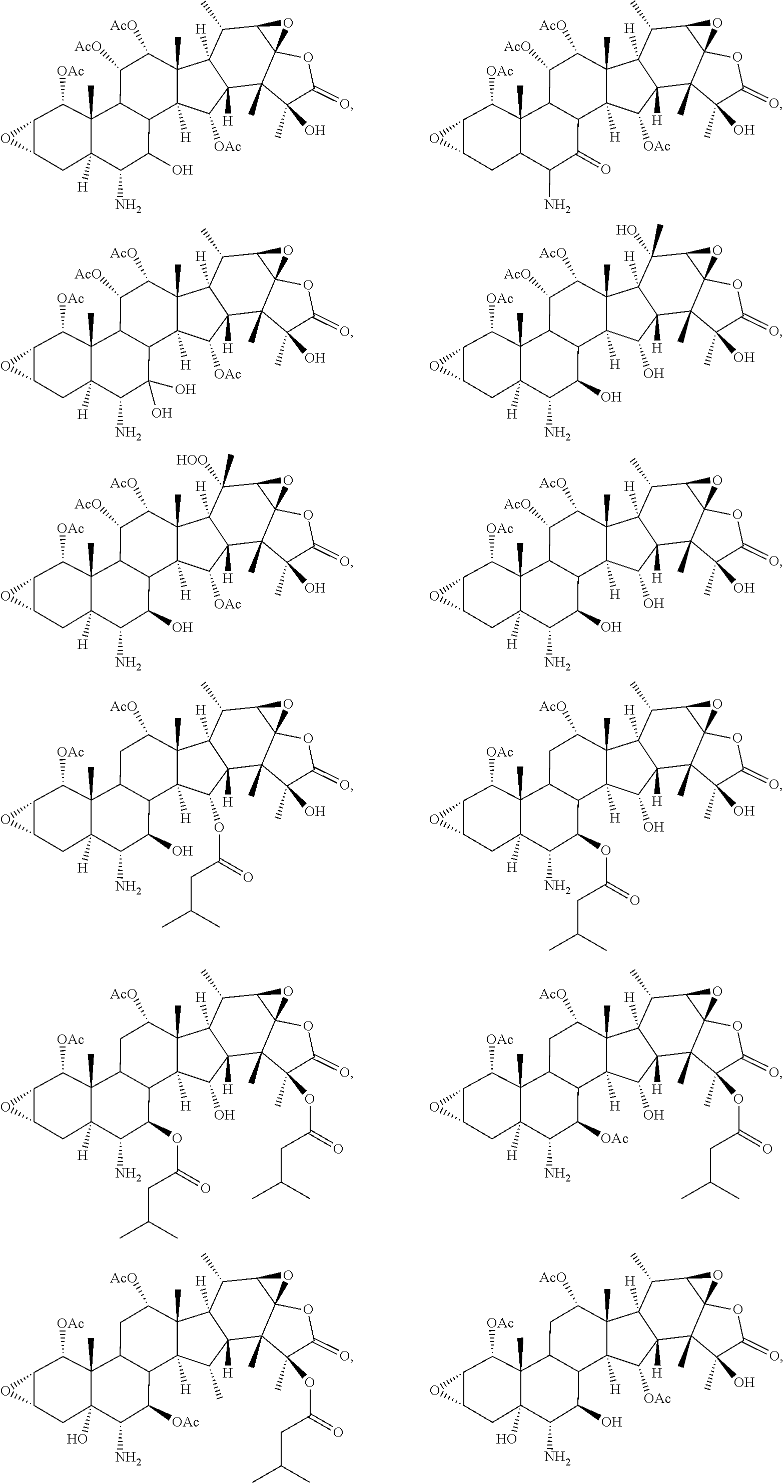

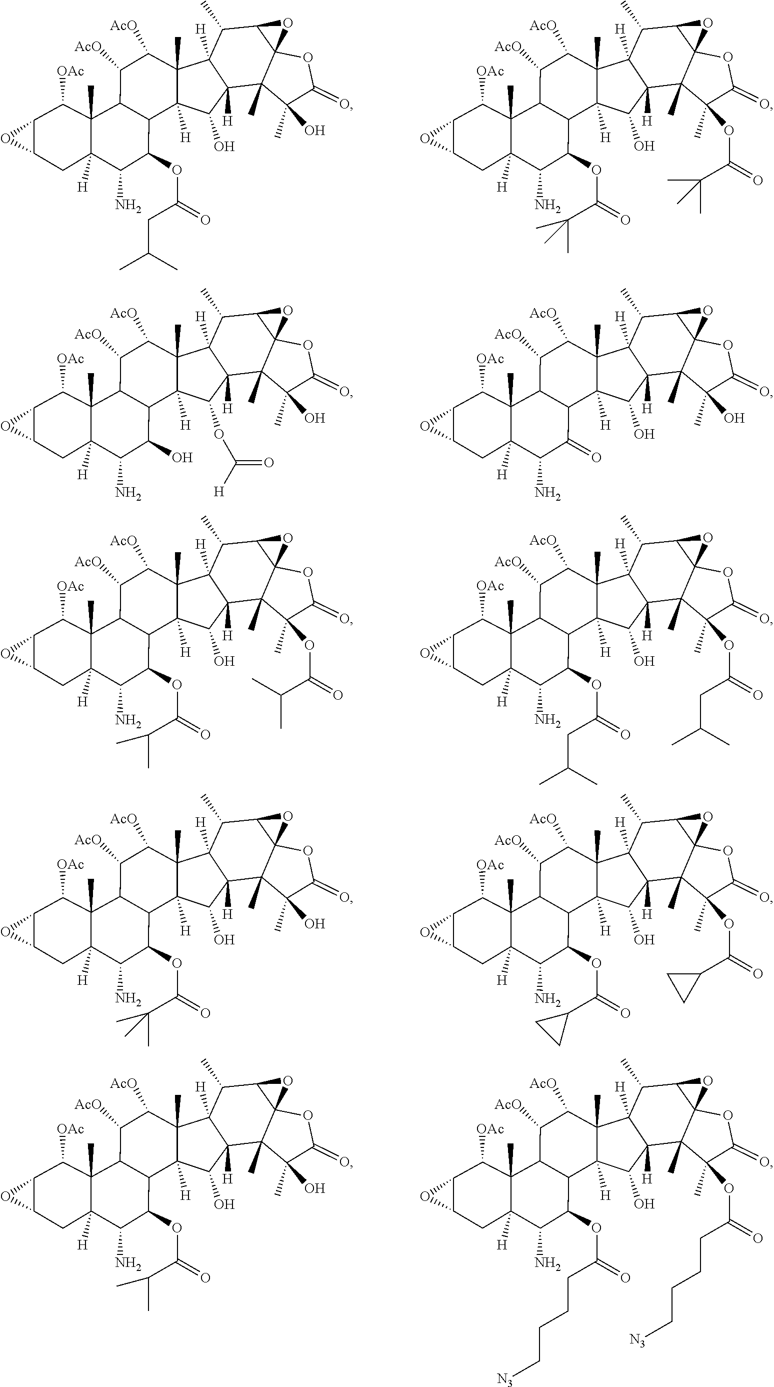

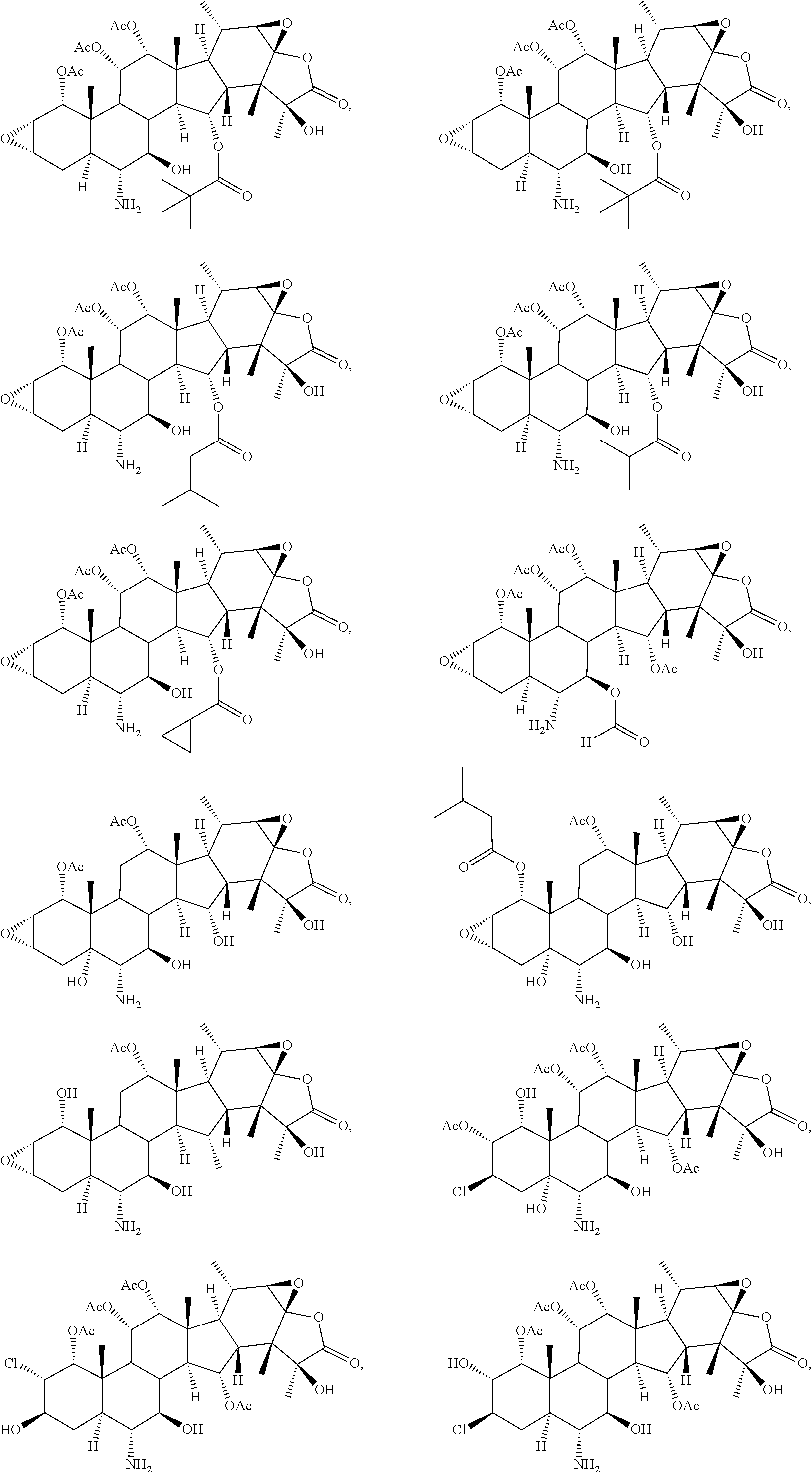

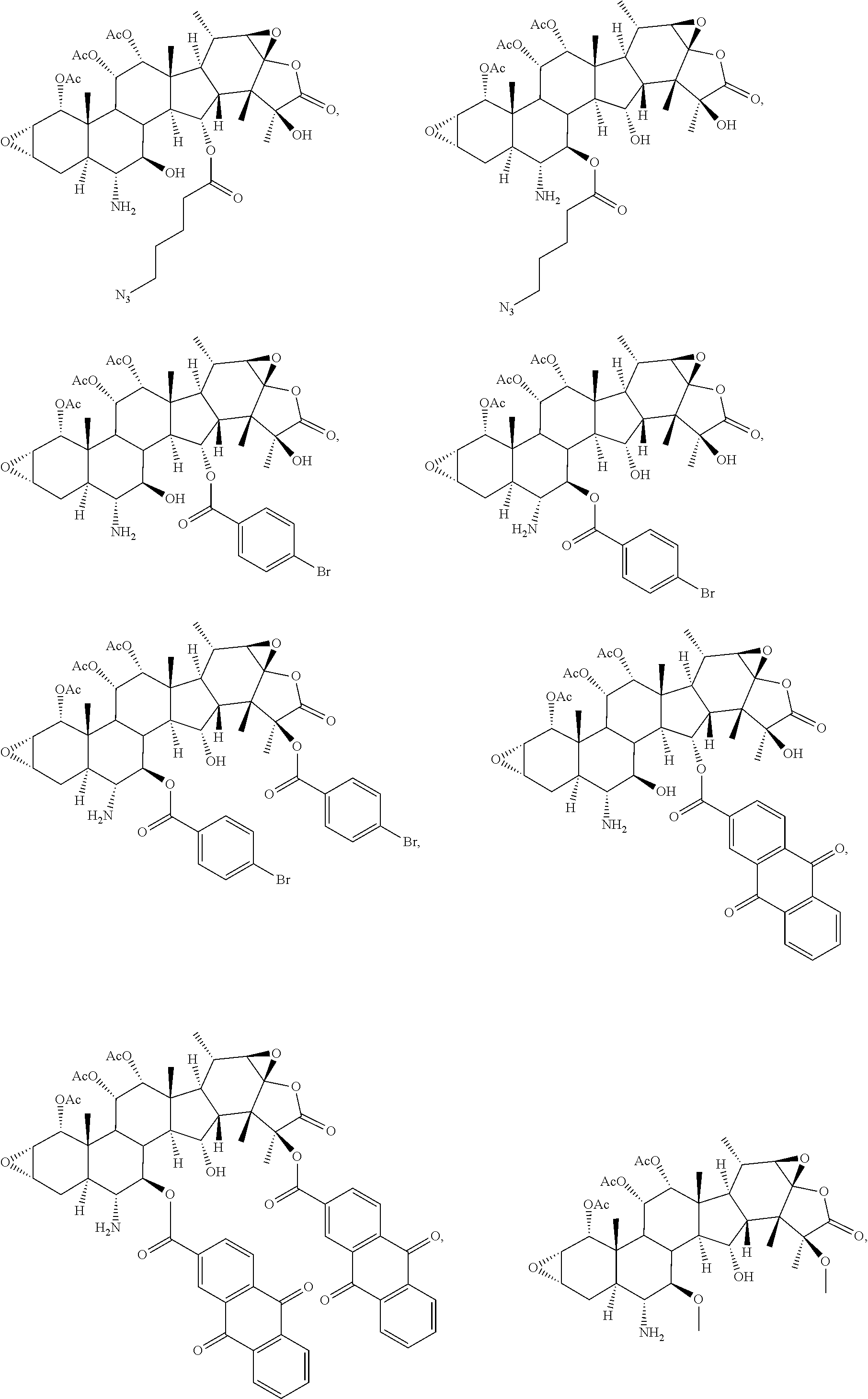

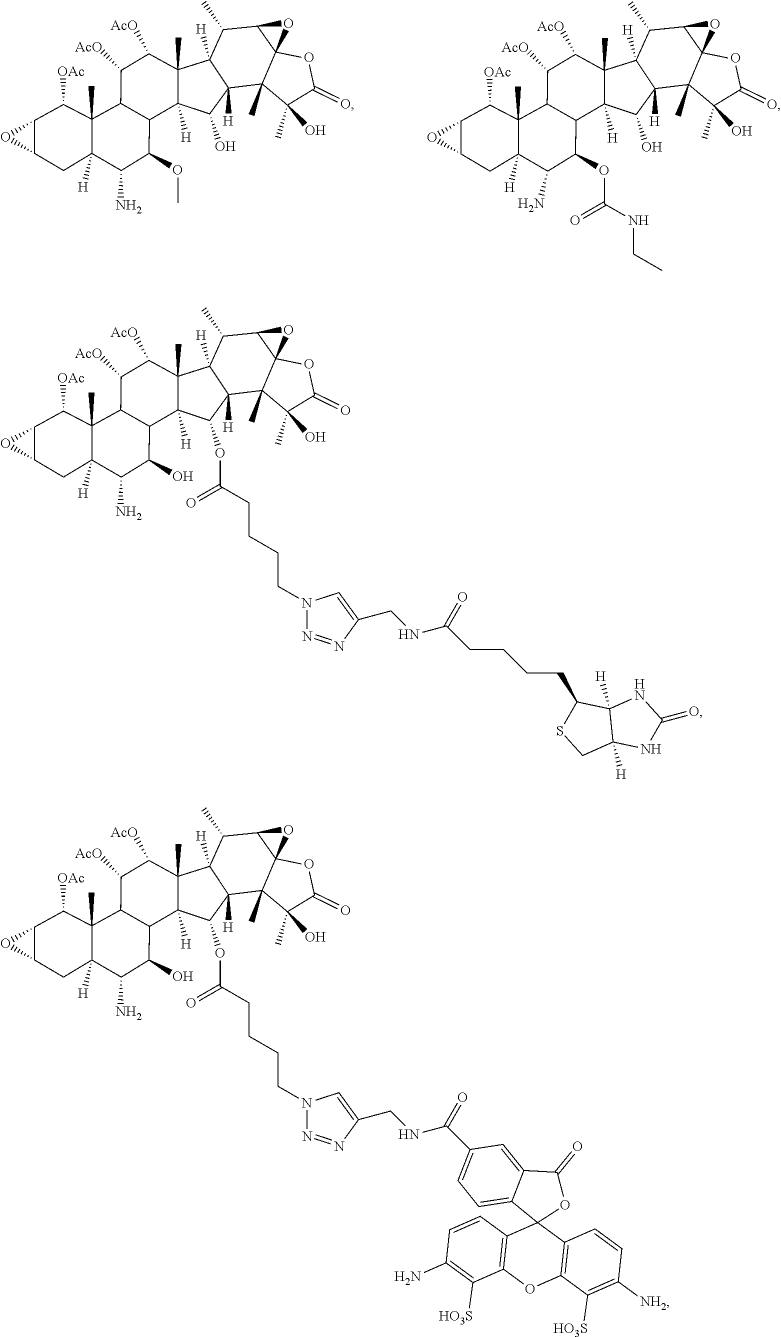

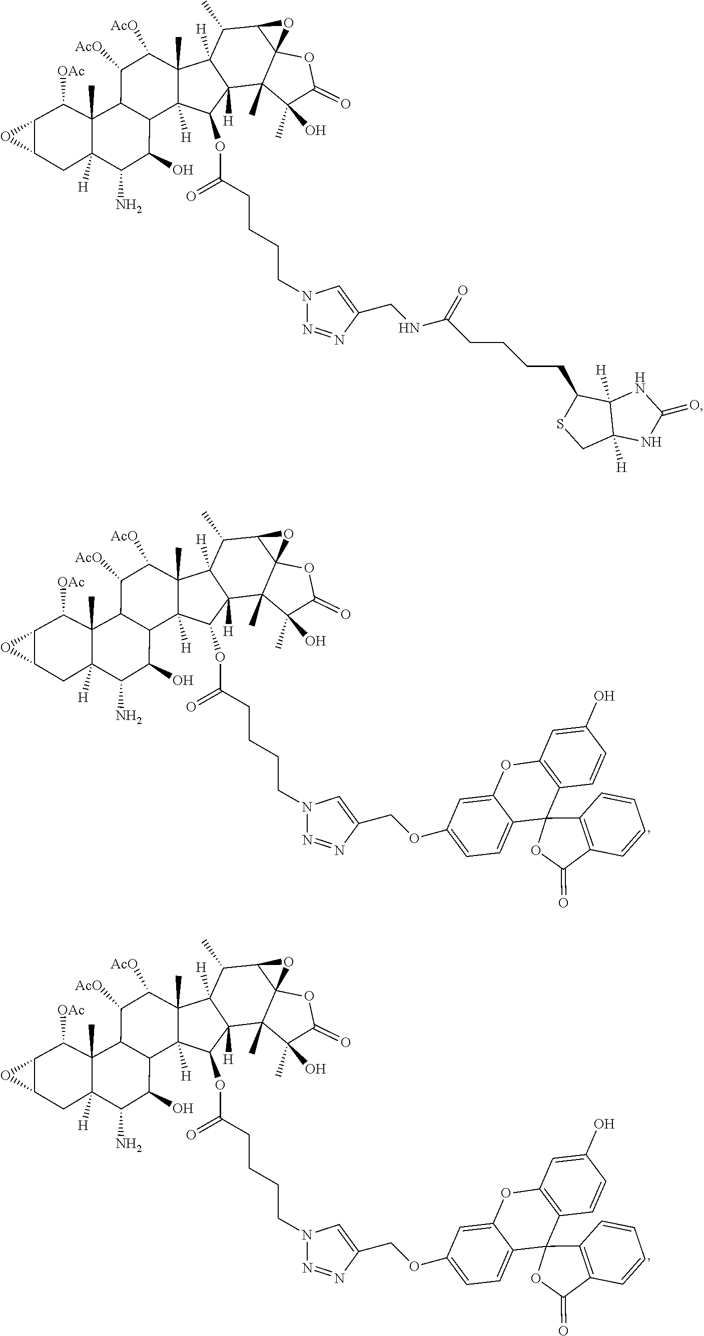

or a pharmaceutically acceptable salt thereof.

In a further aspect, the compound has a structure represented by a formula:

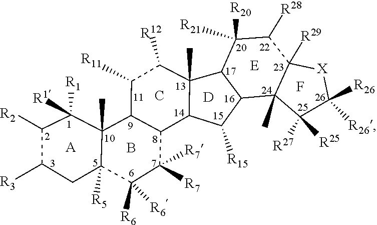

wherein R¹ is selected from OH, C1-C12 alkoxy, and —OC(O)(C1-C12 alkyl); wherein each of R² and R³ is independently selected from —OH and halogen; or wherein each of R₂ and R₃ together comprise —O—; wherein R⁵ is selected from hydrogen, —OH, —NH₂, C1-C9 alkoxy, C1-C6 alkylamino, and (C1-C6)(C1-C6) dialkylamino; or wherein R⁵ is absent; wherein each of R⁶ and R^{6′} is independently selected from hydrogen, —OH, C1-C30 alkoxy, —OC(O)(C1-C30 alkyl), —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰; or wherein one of R⁶ and R^{6′} is absent; wherein each of R⁷ and R^{7′} is independently selected from hydrogen, —OH, C1-C30 alkoxy, and —OC(O)(C1-C30 alkyl); or wherein each of R⁷ and R^{7′} together comprise ═O; or wherein one of R⁷ and R^{7′} is absent; wherein each of R¹¹ and R¹² is independently selected from hydrogen, —OH, C1-C6 alkyl, C1-C8 alkoxy, and —OC(O)(C1-C8 alkyl); wherein R¹⁵ is selected from hydrogen, —OH, C1-C30 alkyl, C1-C30 alkoxy, and —OC(O)(C1-C30 alkyl); wherein R²⁰ is selected from hydrogen, —OH, —OOH, C1-C8 alkyl, C1-C8 alkoxy, and —OC(O)(C1-C8 alkyl); wherein R²⁵ is selected from hydrogen, —OH, C1-C8 alkoxy, and —OC(O)(C1-C18 alkyl); and wherein each of R²⁶ and R^{26′} is independently selected from hydrogen, —OH, and C1-C8 alkoxy; or wherein each of R²⁶ and R^{26′} together comprise ═O, provided that at least one of R⁶ and R^{6′} is —NR⁴¹C(O)(C1-C30 alkyl), —NR⁴¹C(O)Ar², —NR⁴¹C(O)(C1-C30 alkyl)Ar², —NR⁴¹C(O)Ar³, —NR4′C(O)(C1-C30 alkyl)OC(O)Ar³, —NR⁴¹C(O)(C1-C30 alkyl)NR⁴²C(O)Ar³, or —NR⁴¹C(O)R⁴⁰, or a pharmaceutically acceptable salt thereof.

In a further aspect, the compound has structure represented by a formula:

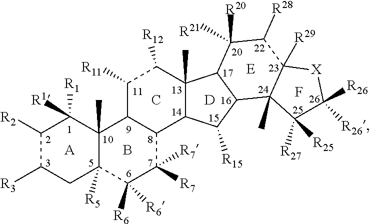

wherein R¹ is selected from —OH, C1-C12 hydroxy, C1-C12 alkoxy, and —OC(O)(C1-C12 alkyl); wherein R⁵ is selected from hydrogen, —OH, —NH₂, C1-C6 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; wherein each of R⁶ and R^{6′} is independently selected from hydrogen, —OH, C1-C30 hydroxy, C1-C30 alkoxy, —OC(O)(C1-C30 alkyl), —OC(O)Ar², —OC(O)(C1-C30 alkyl)Ar², —OC(O)R⁴⁰, —NR⁴¹C(O)(C1-C30 alkyl), —NR4′C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR4′C(O)R⁴⁰; or wherein one of R⁶ and R^{6′} is absent; wherein each of R⁷ and R^{7′} is independently selected from hydrogen, —OH, C1-C30 hydroxy, C1-C30 alkoxy, and —OC(O)(C1-C30 alkyl); or wherein each of R⁷ and R^{7′} together comprise ═O; or wherein one of R⁷ and R^{7′} is absent, provided that at least one of R⁶ and R^{6′} is —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, or —NR⁴¹C(O)R⁴⁰, or a pharmaceutically acceptable derivative thereof.

In a further aspect, the compound has a structure represented by a formula:

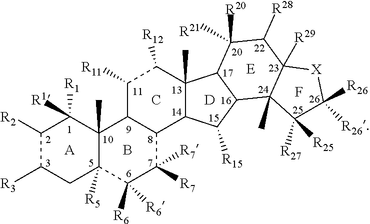

In a further aspect, the compound has a structure represented by a formula:

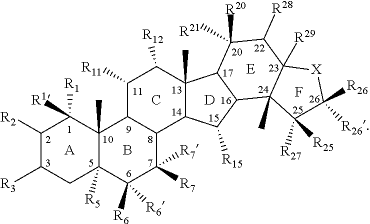

In a further aspect, the compound has a structure represented by a formula:

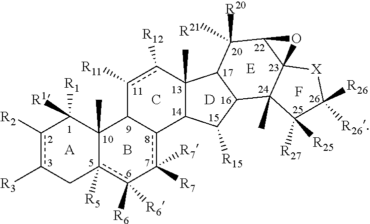

In a further aspect, the compound has a structure represented by a formula:

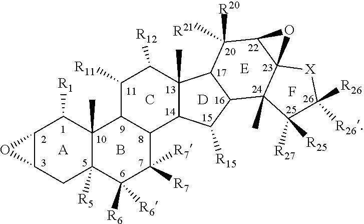

In a further aspect, the compound has a structure represented by a formula:

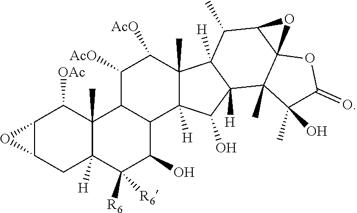

In a further aspect, the compound has a structure represented by a formula:

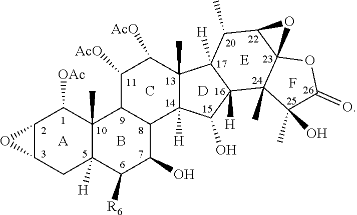

In a further aspect, the compound has a structure represented by a formula:

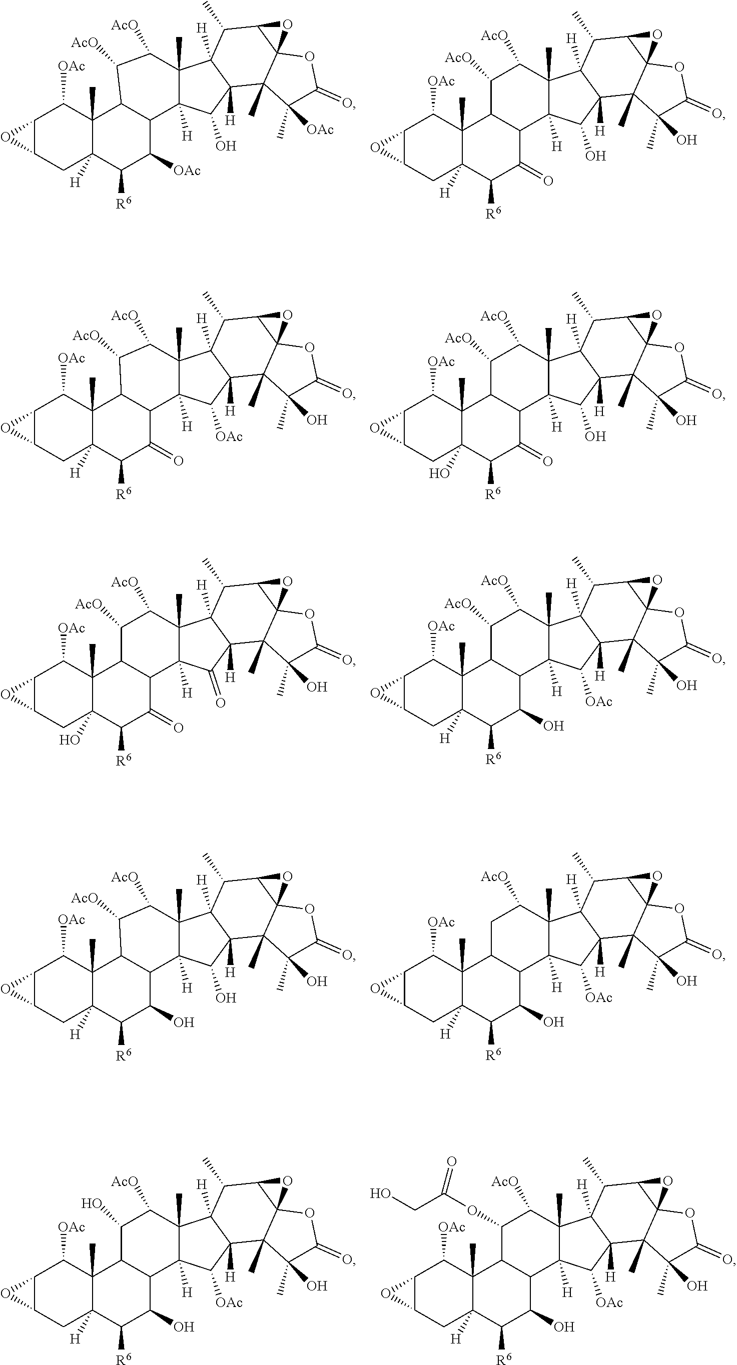

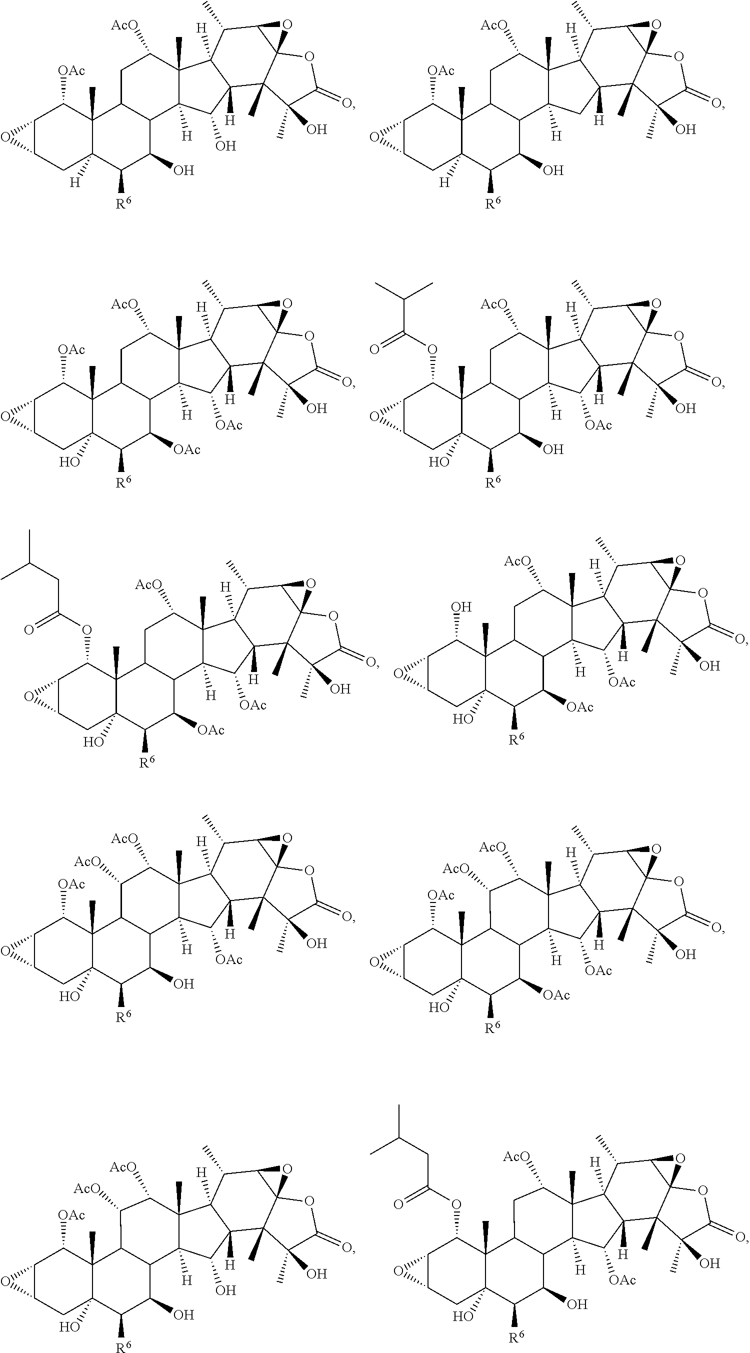

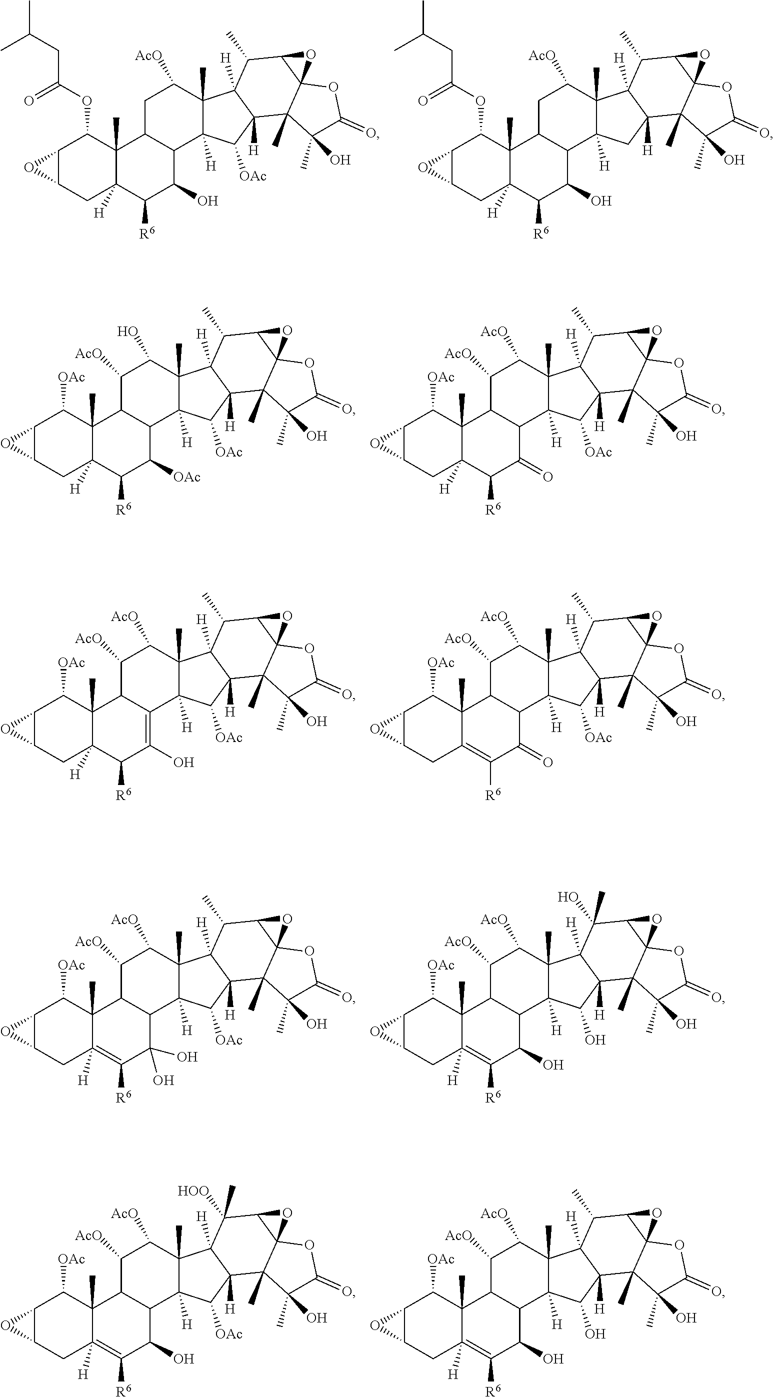

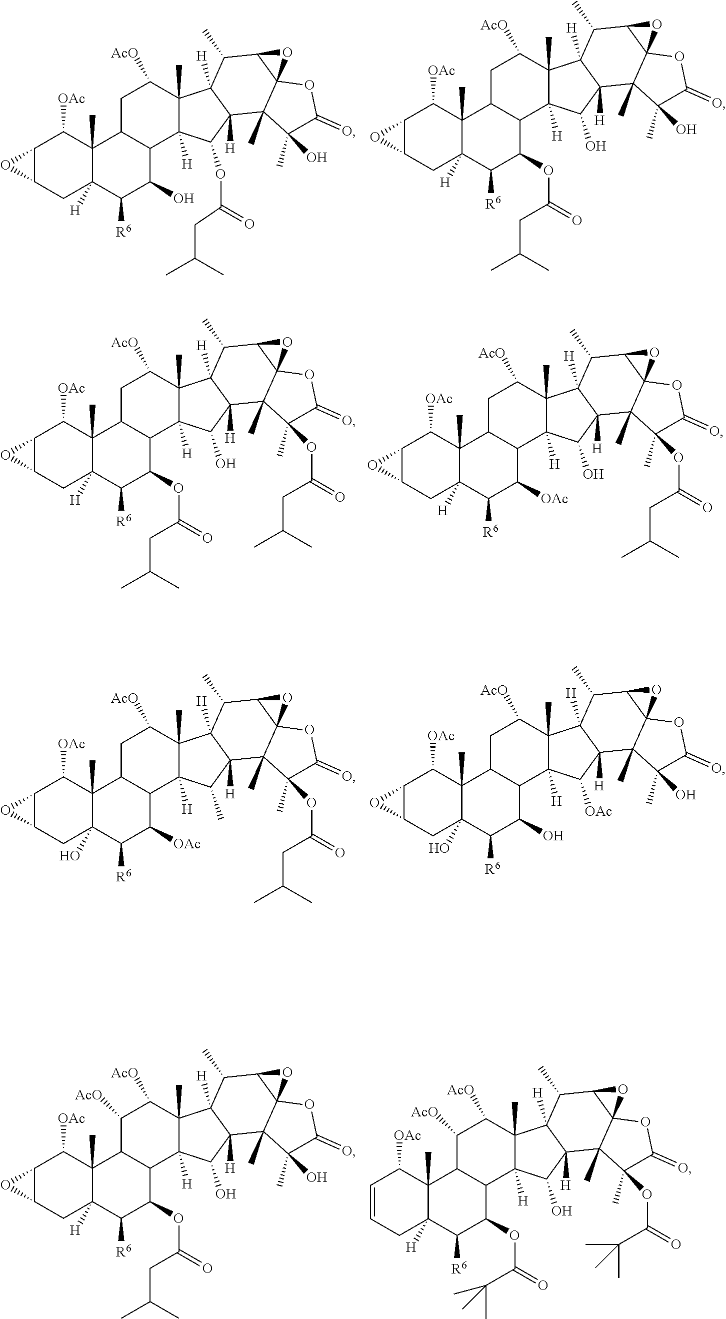

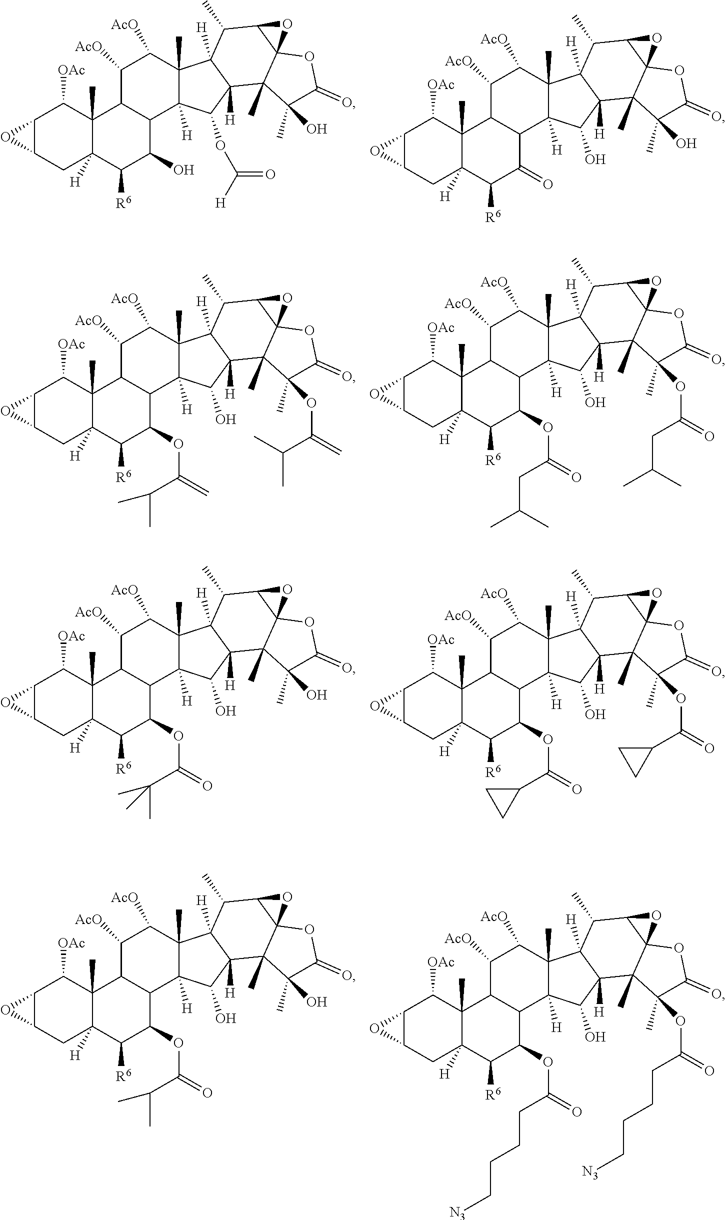

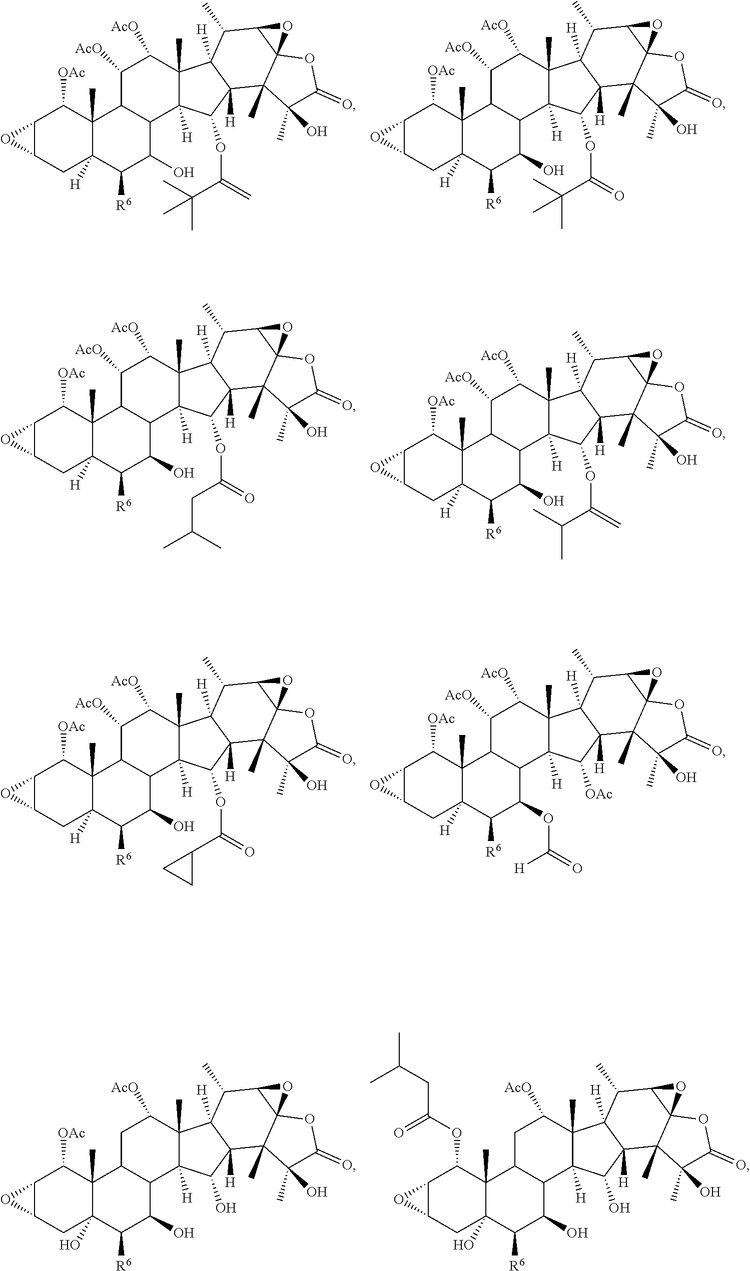

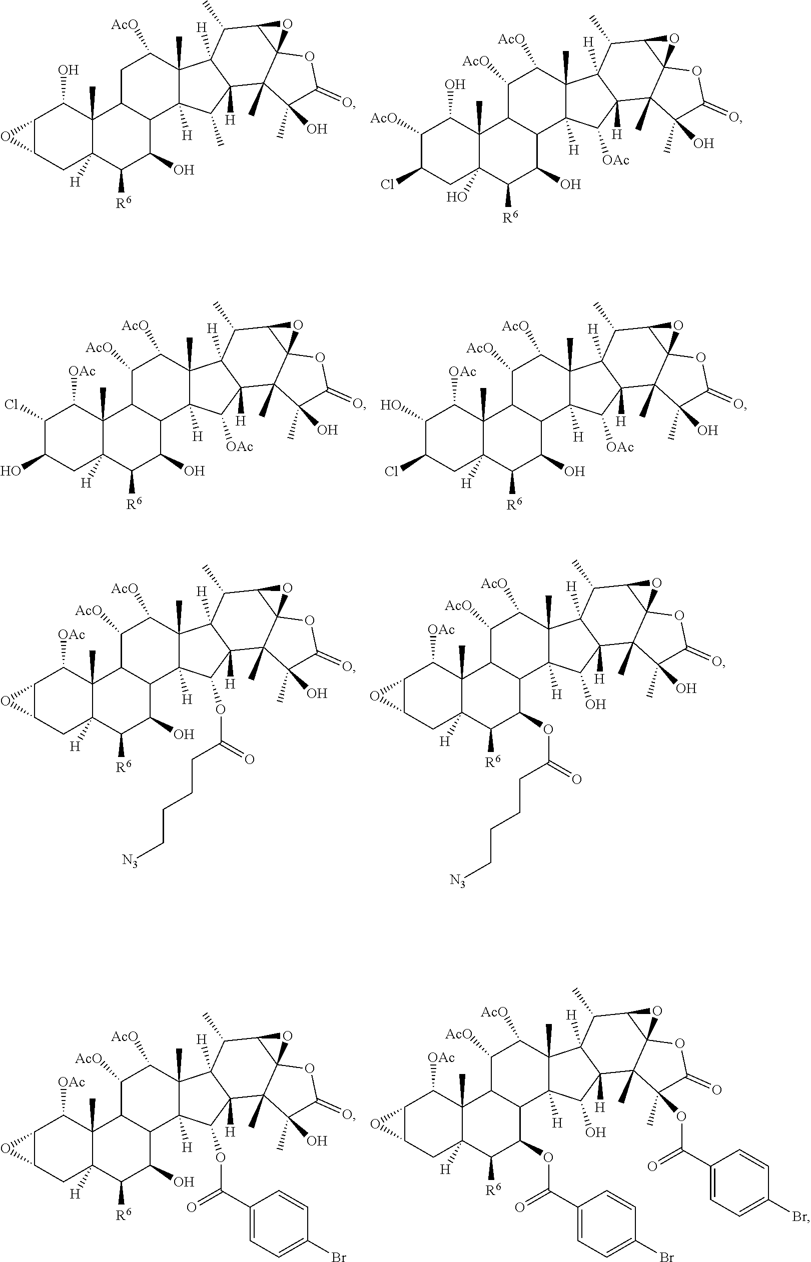

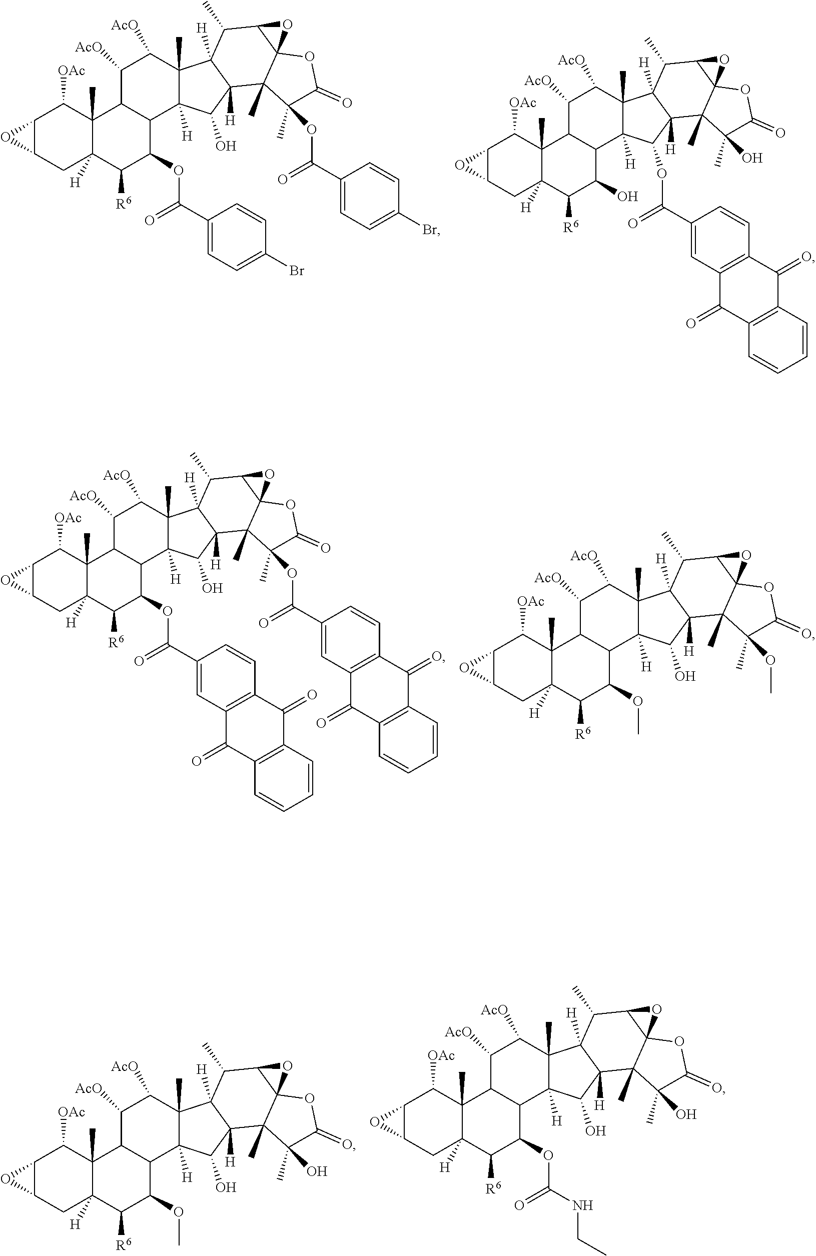

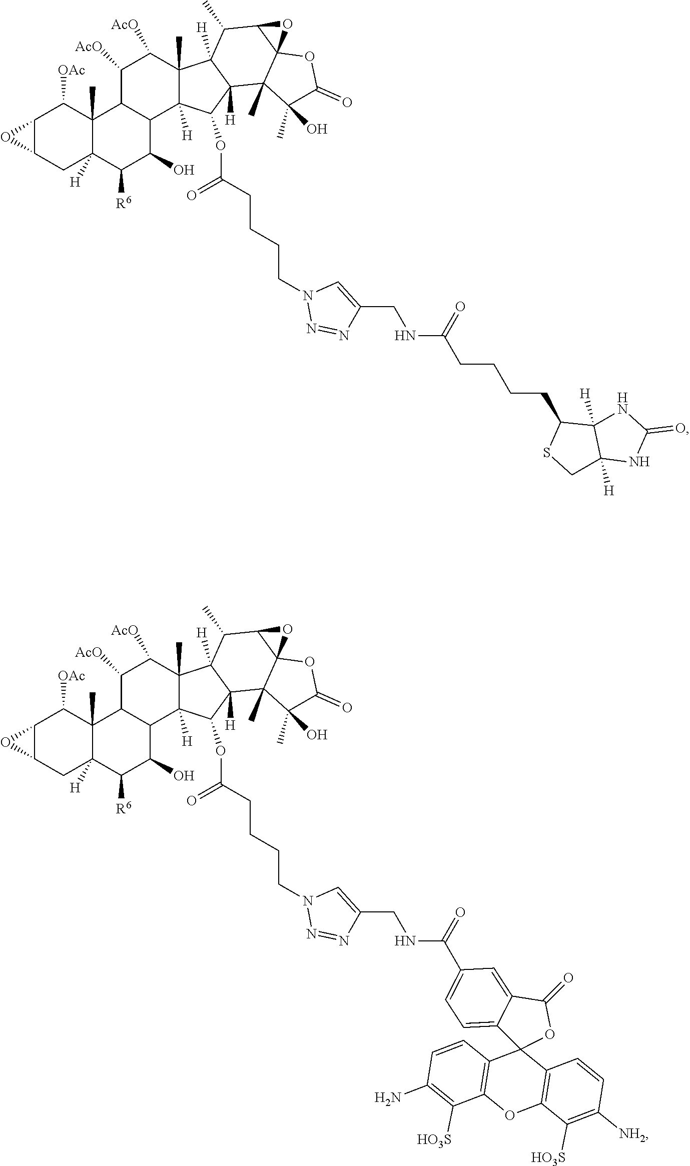

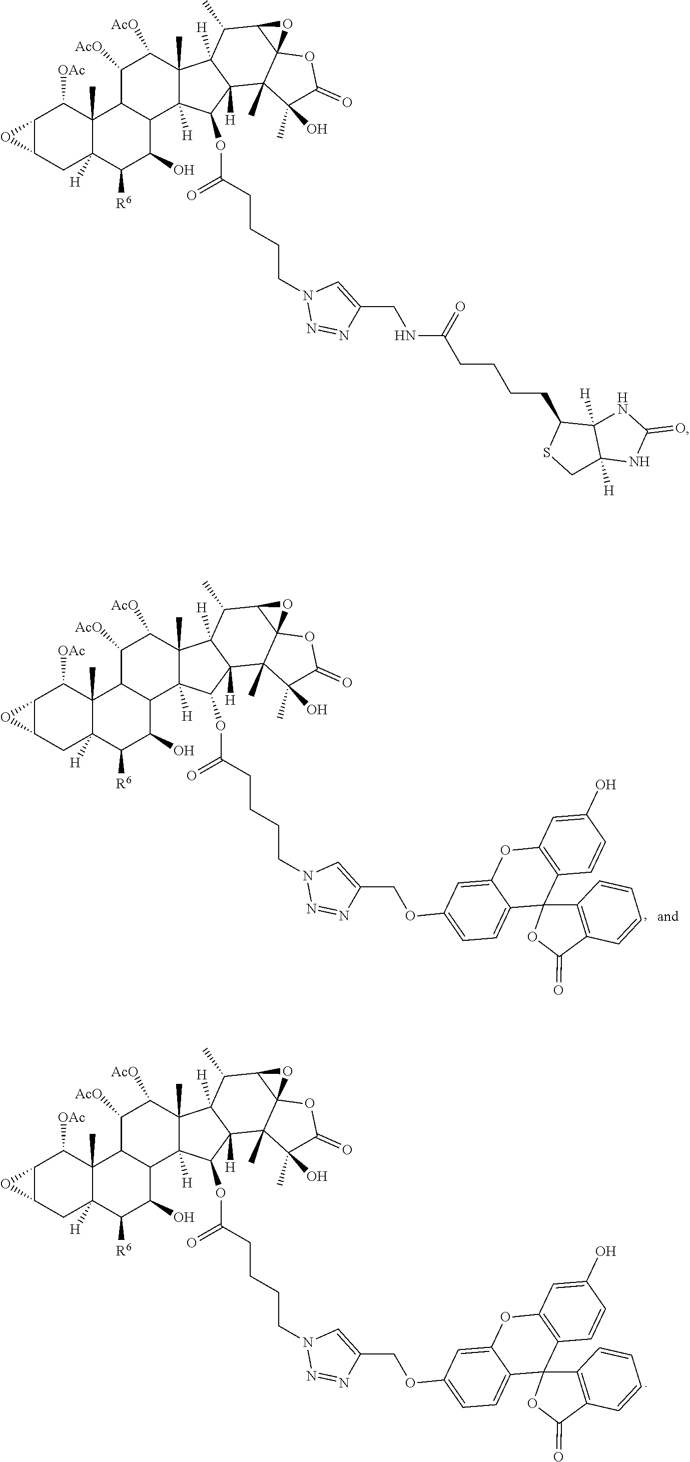

In a further aspect, the compound has a structure represented by a formula:

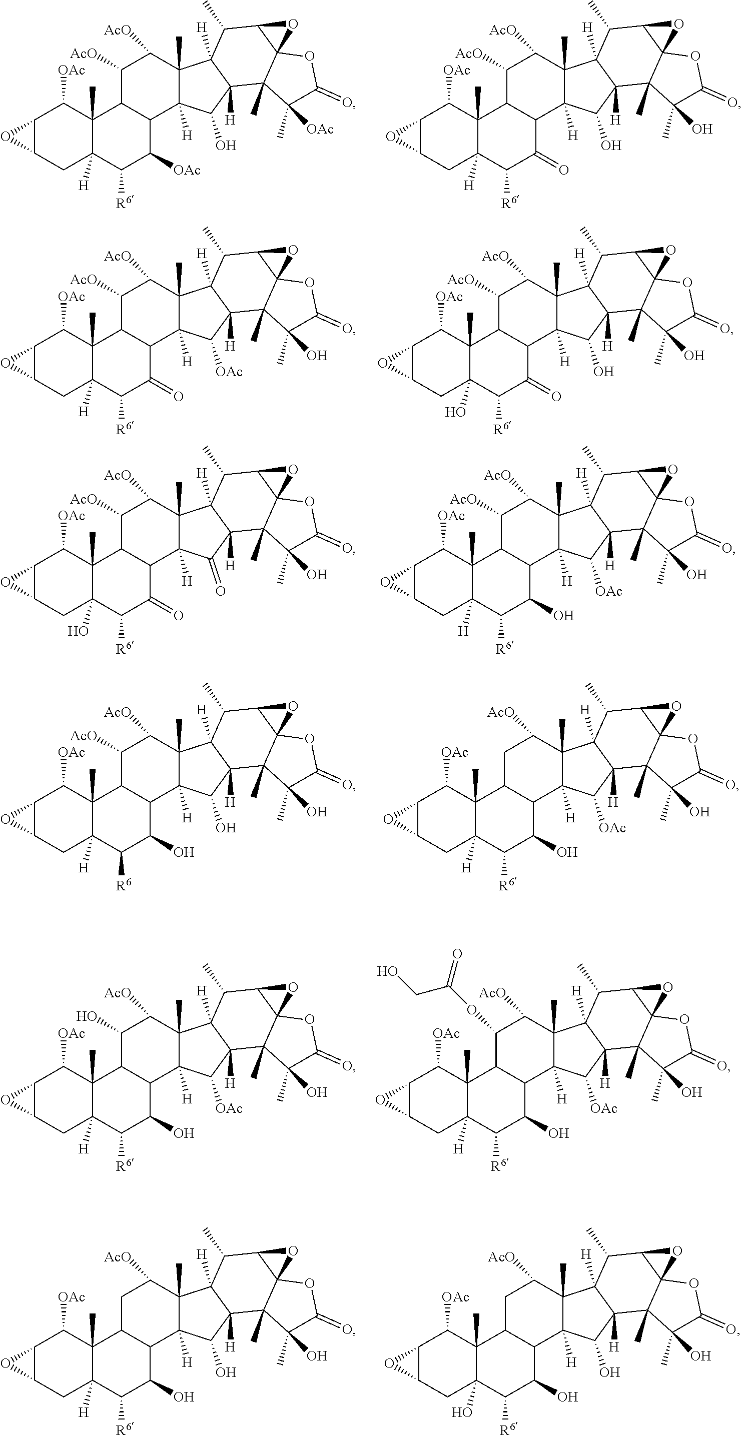

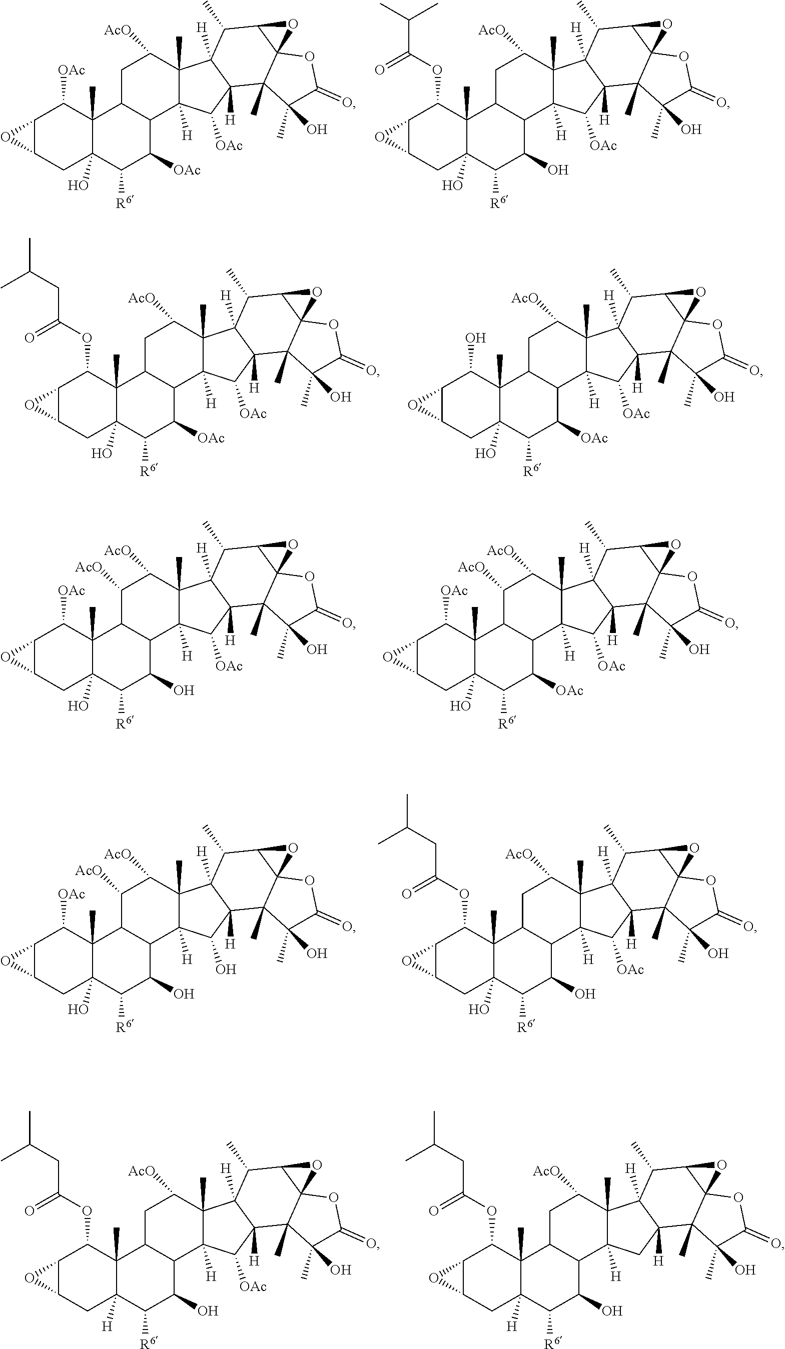

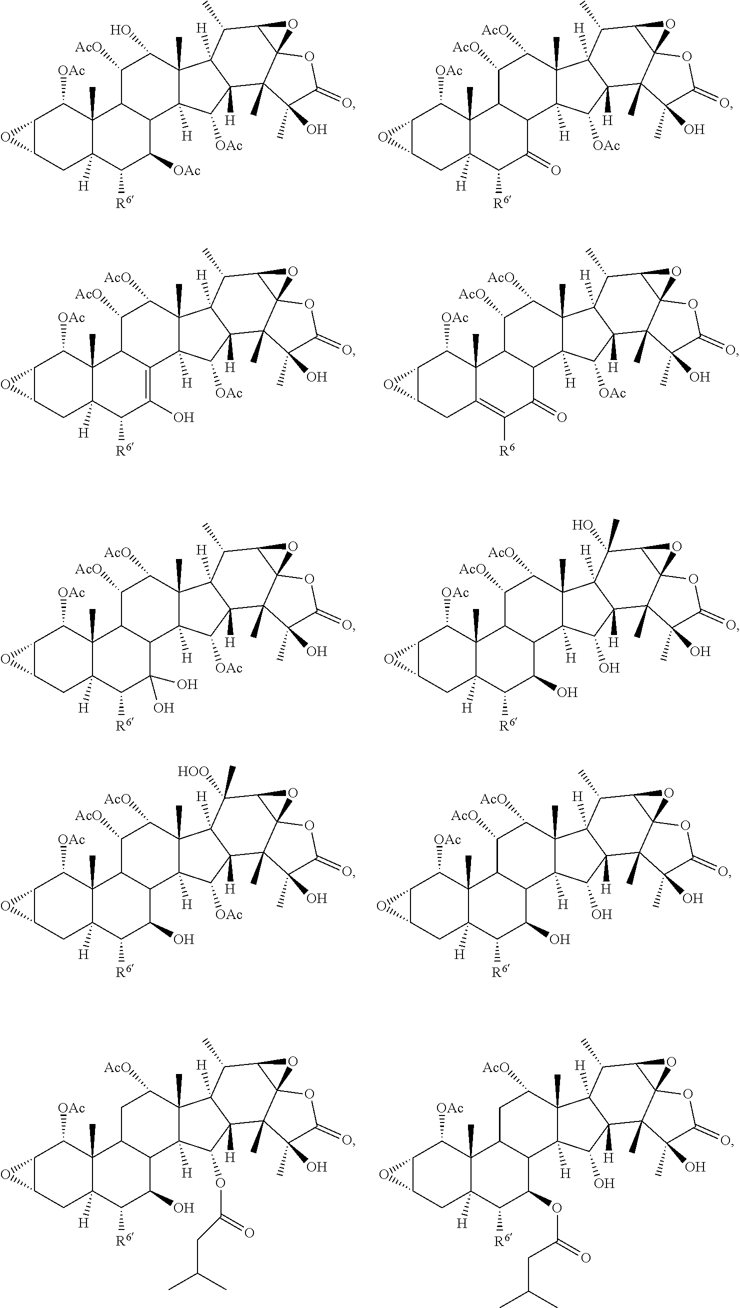

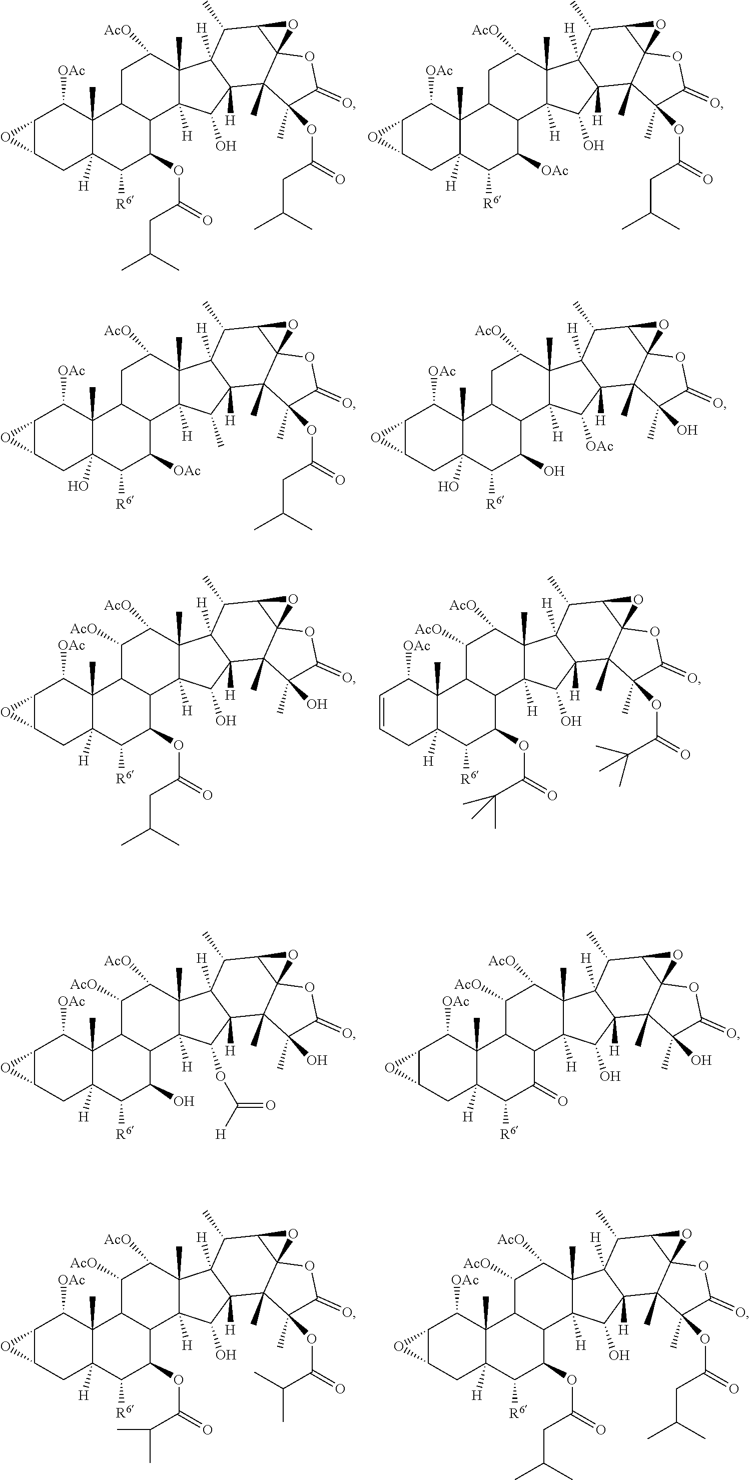

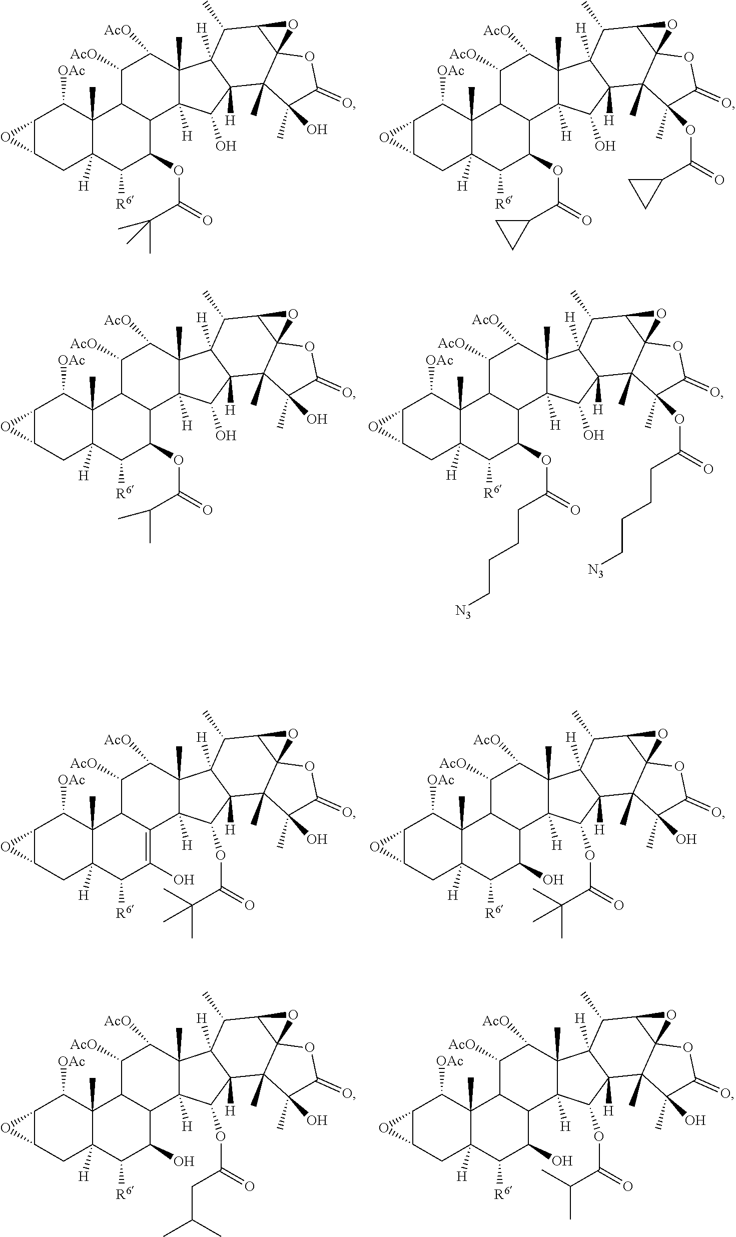

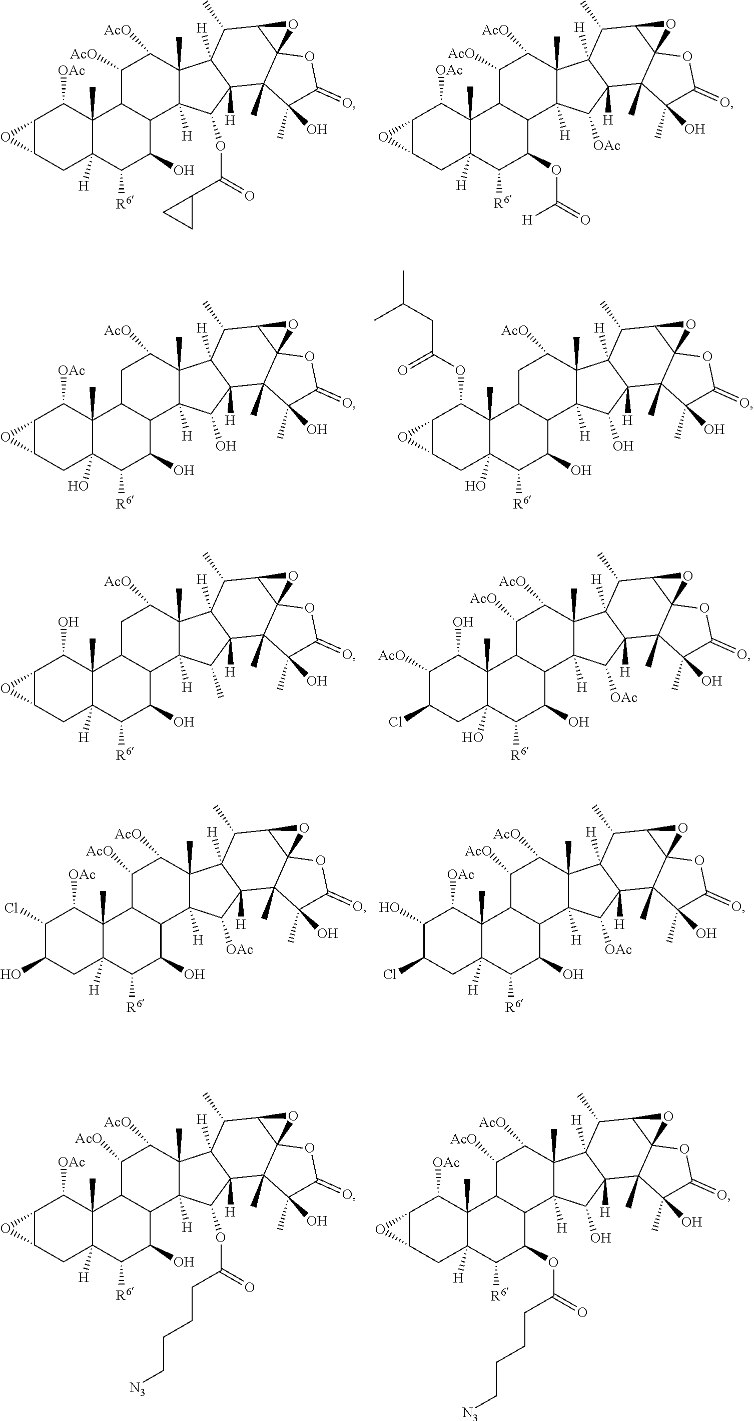

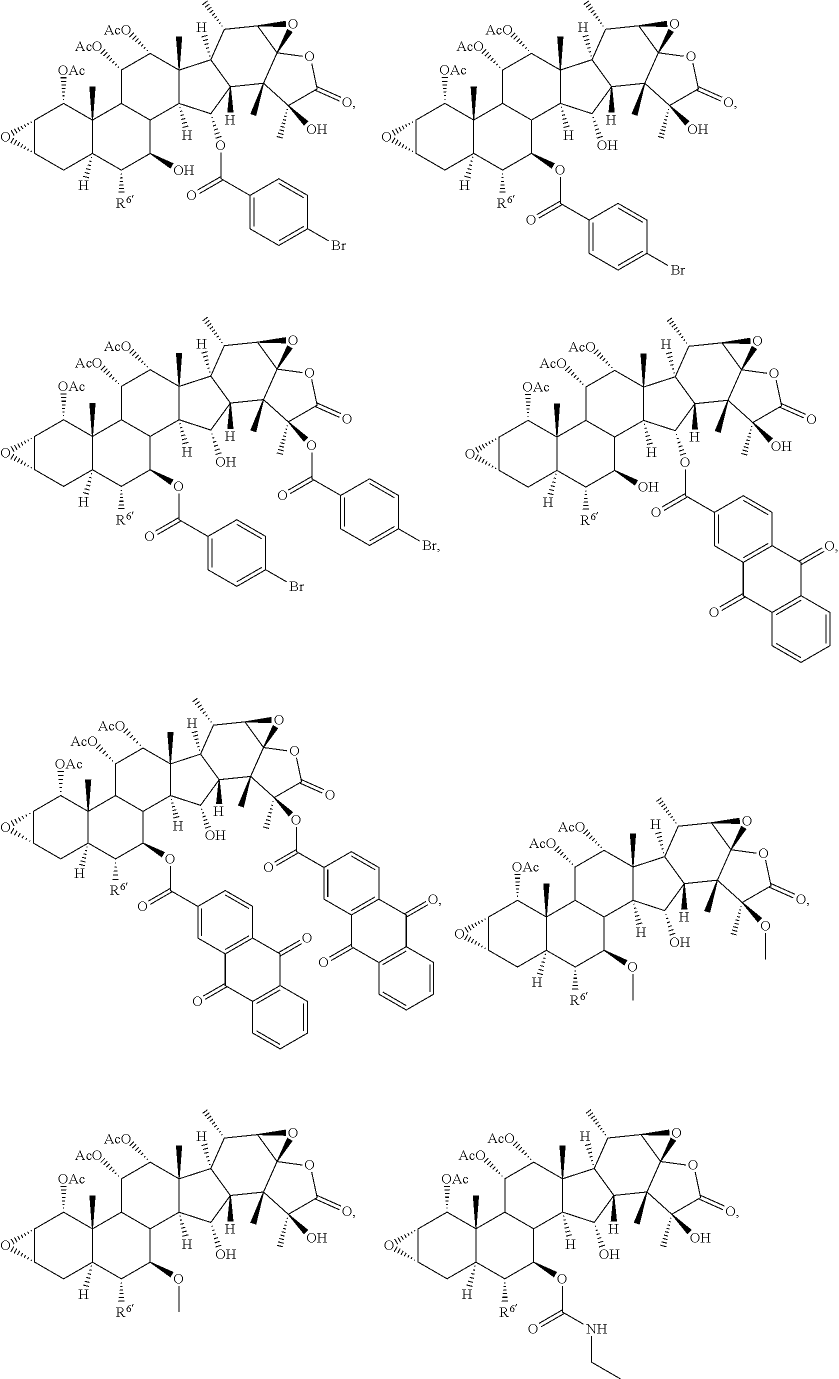

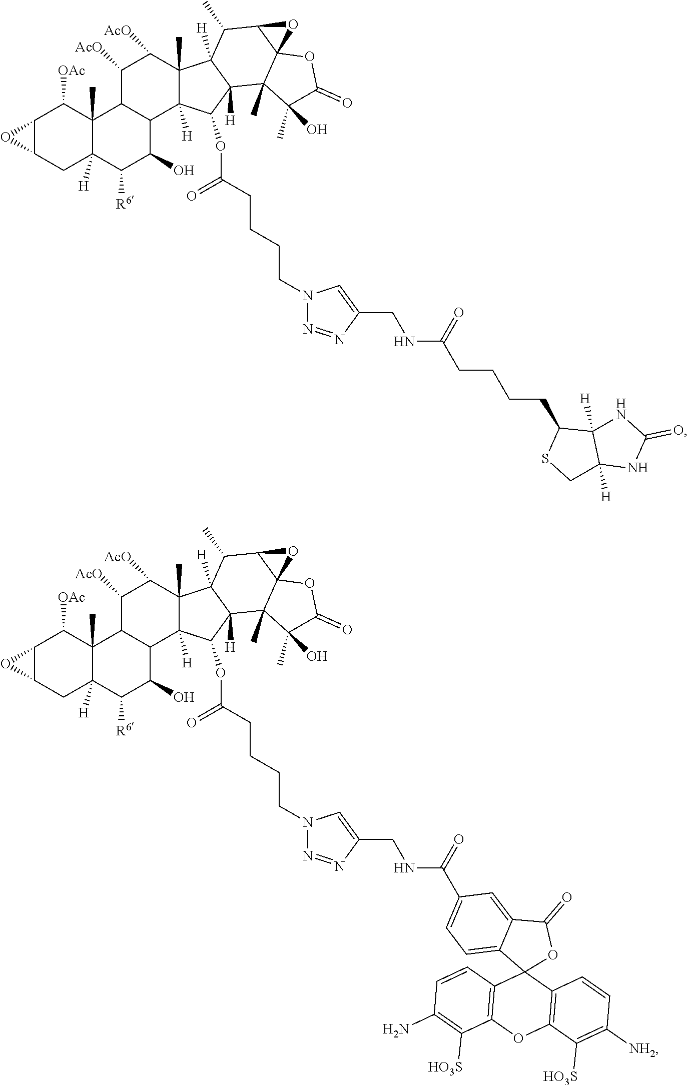

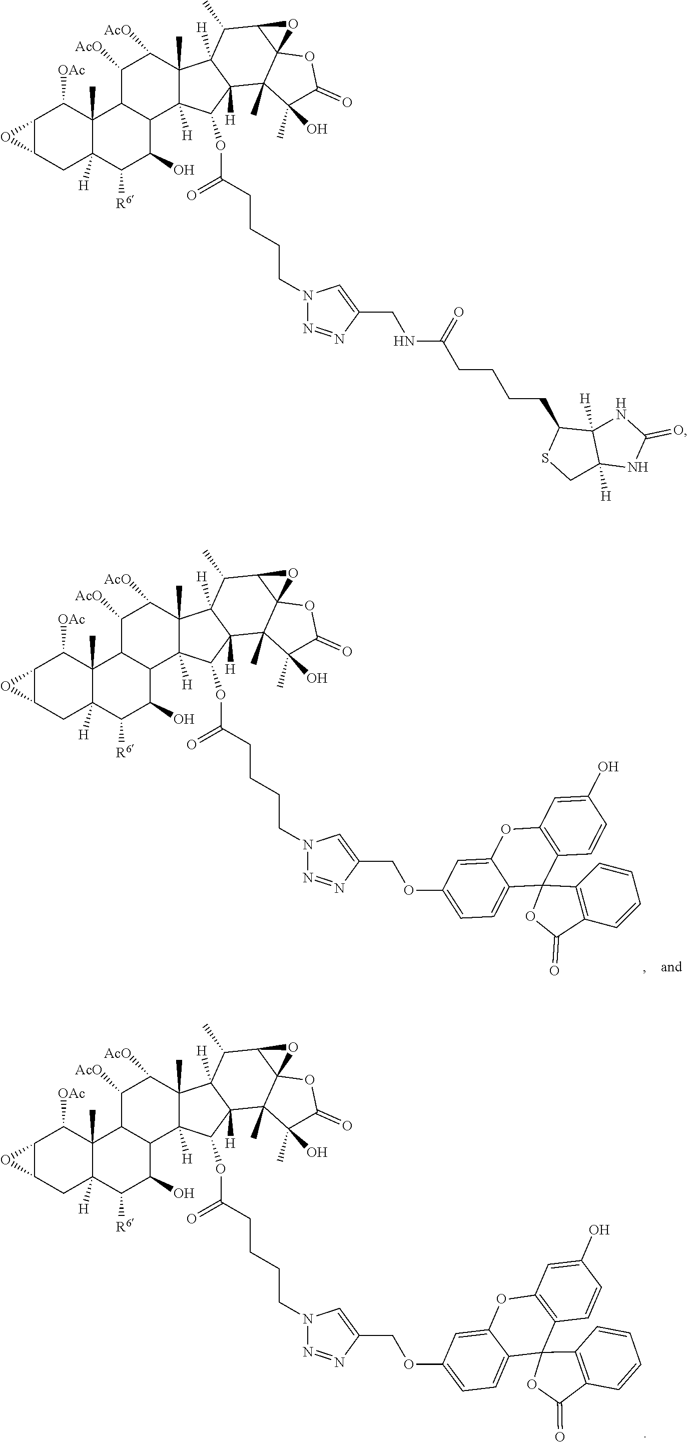

In a further aspect, the compound has a structure selected from:

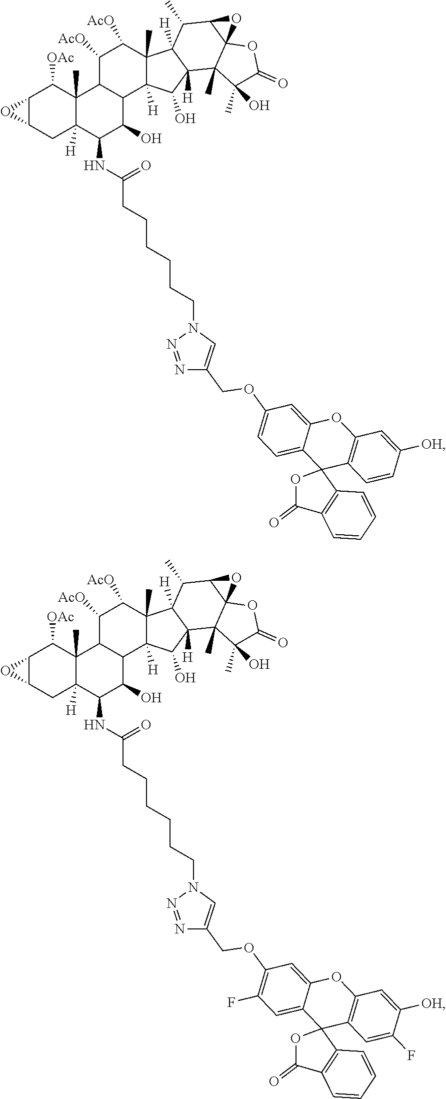 [SMILES: CC(=O)O[C@H]1C2C([C@@H](O)[C@@H](NC(=O)CCCCCCn3cc(COc4ccc5c(c4)Oc4cc(O)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@@H](NC(=O)CCCCCCn3cc(COc4cc5c(cc4F)C4(OC(=O)c6ccccc64)c4cc(F)c(O)cc4O5)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

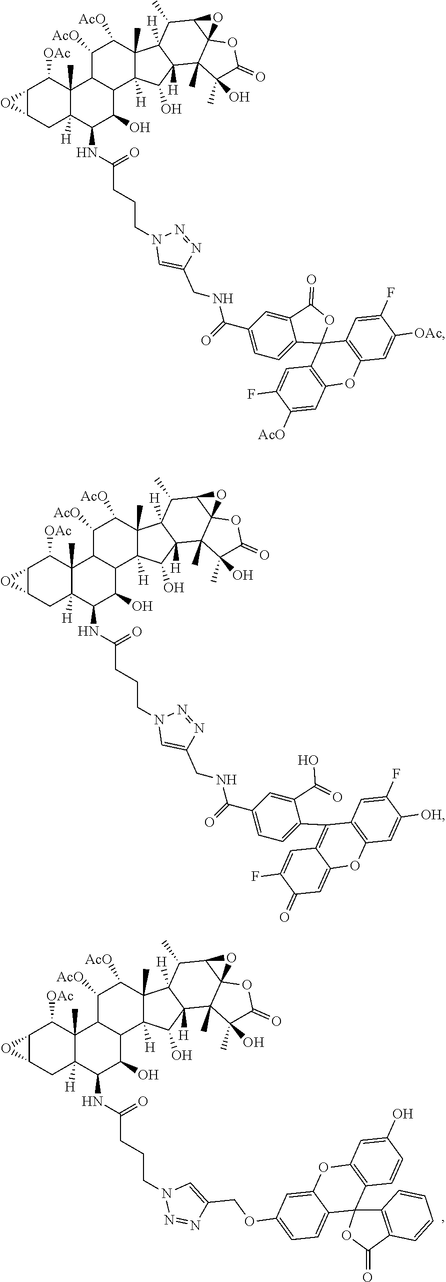

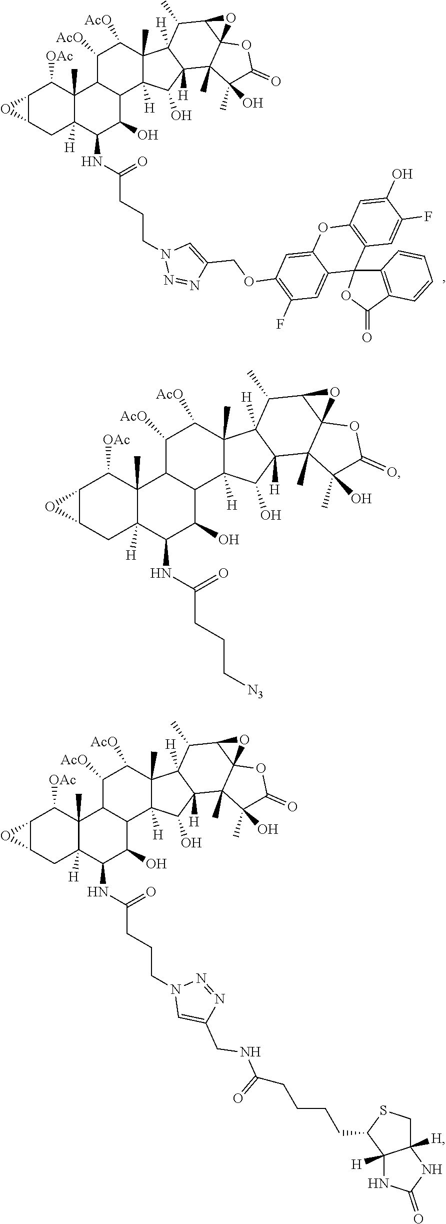

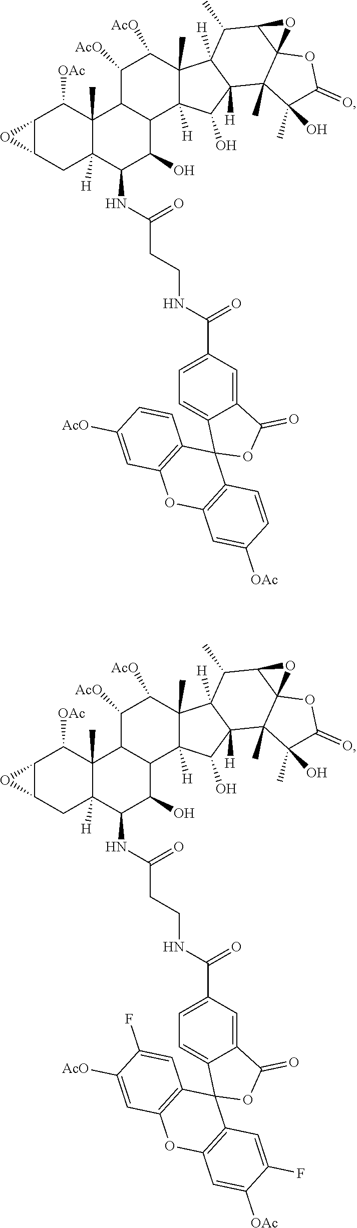

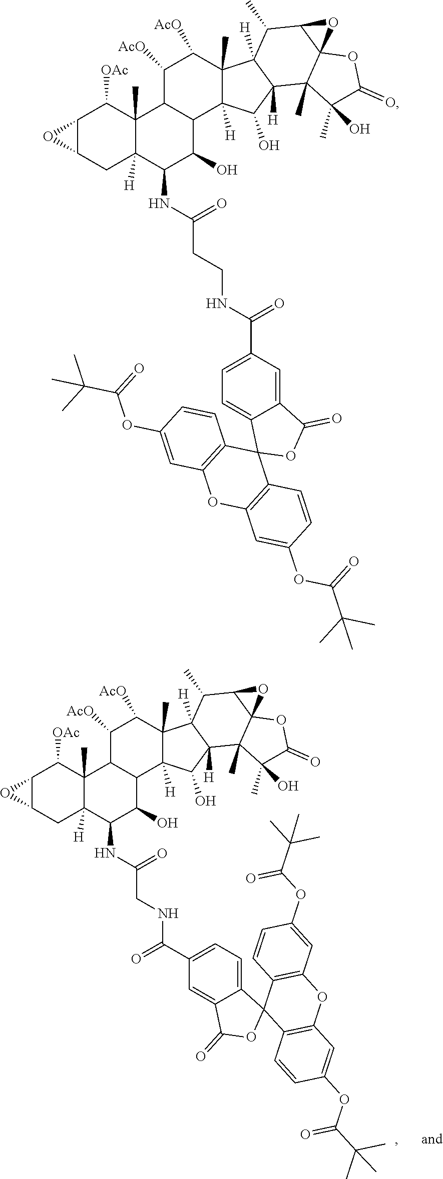

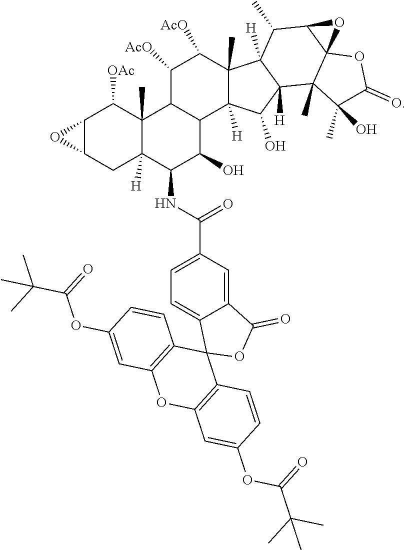 [SMILES: CC(=O)O[C@H]1C2C([C@@H](O)[C@@H](NC(=O)c3ccc4c(c3)C(=O)OC43c4ccc(OC(=O)C(C)(C)C)cc4Oc4cc(OC(=O)C(C)(C)C)ccc43)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

In a further aspect, the compound has a structure selected from:

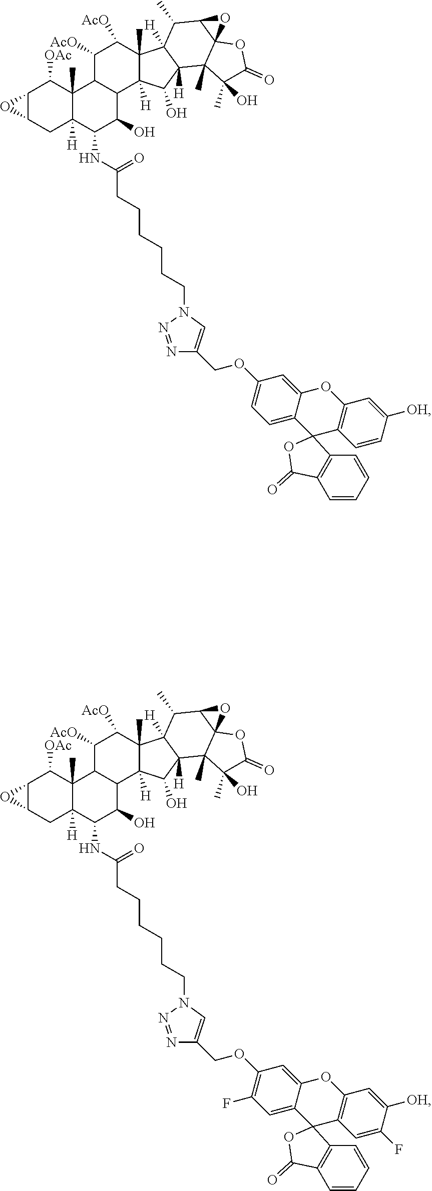 [SMILES: CC(=O)O[C@H]1C2C([C@@H](O)[C@H](NC(=O)CCCCCCn3cc(COc4ccc5c(c4)Oc4cc(O)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@H](NC(=O)CCCCCCn3cc(COc4cc5c(cc4F)C4(OC(=O)c6ccccc64)c4cc(F)c(O)cc4O5)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

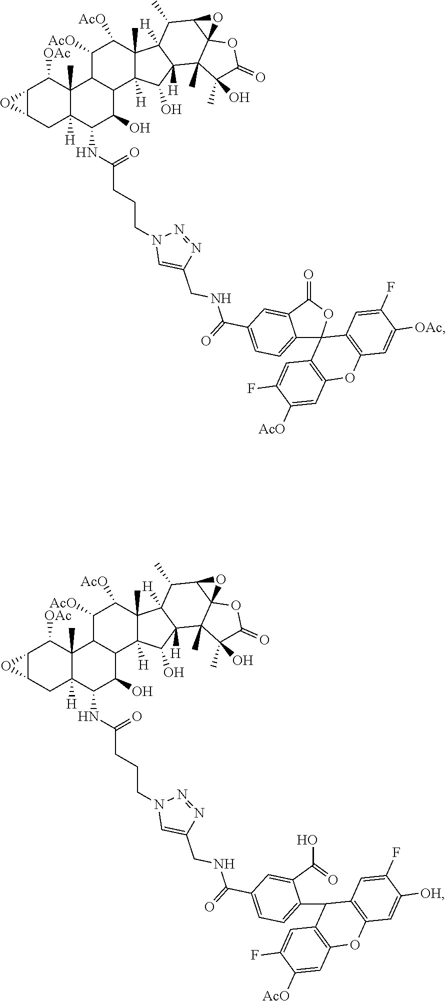

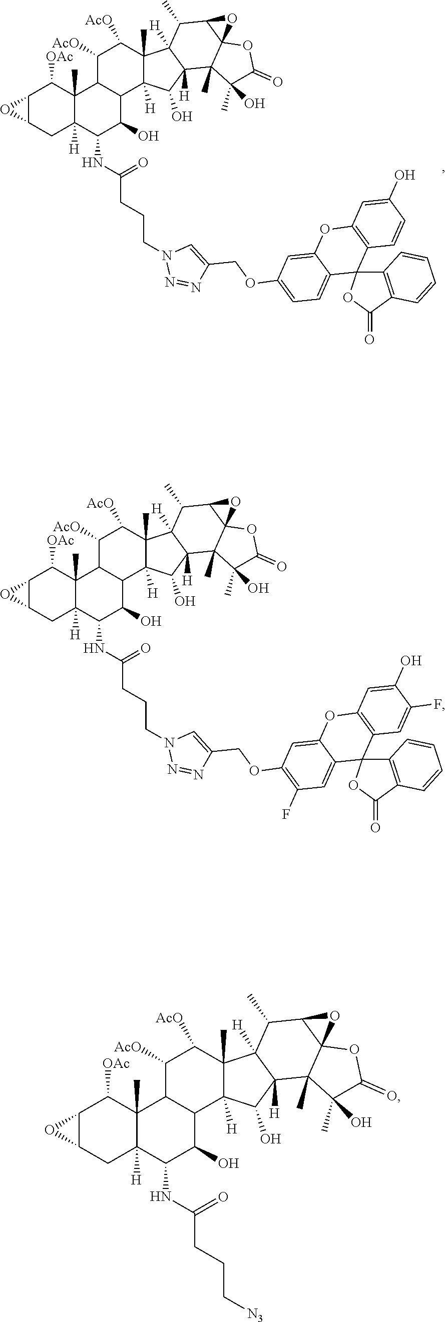

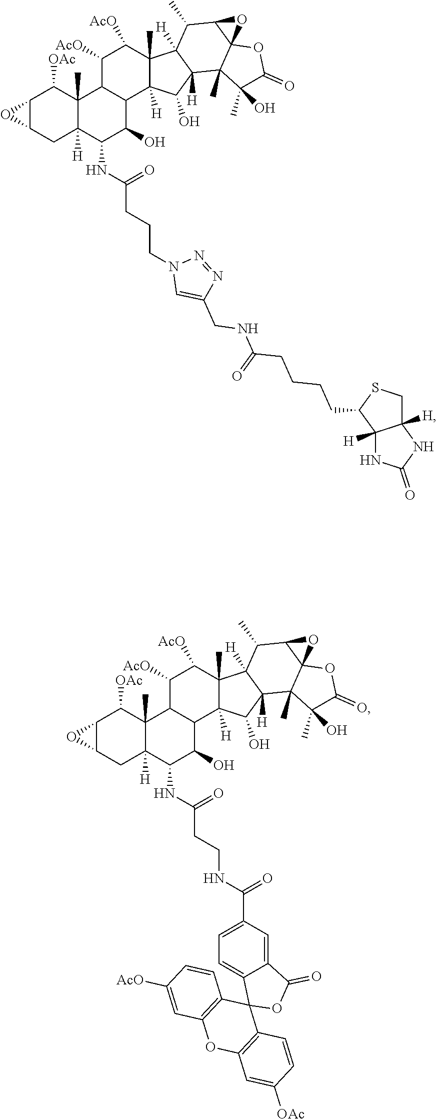

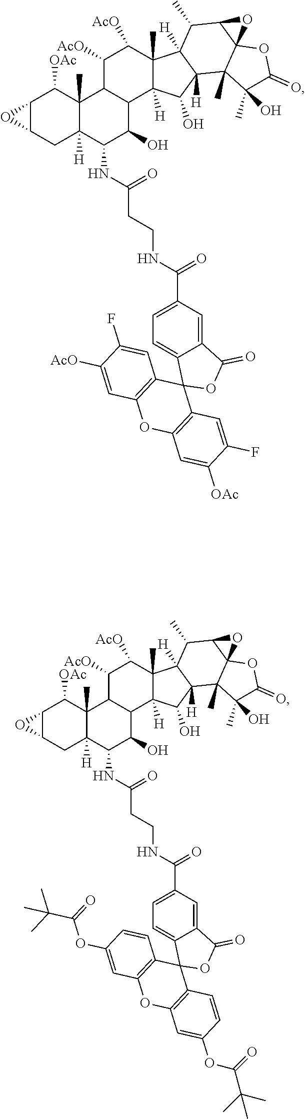

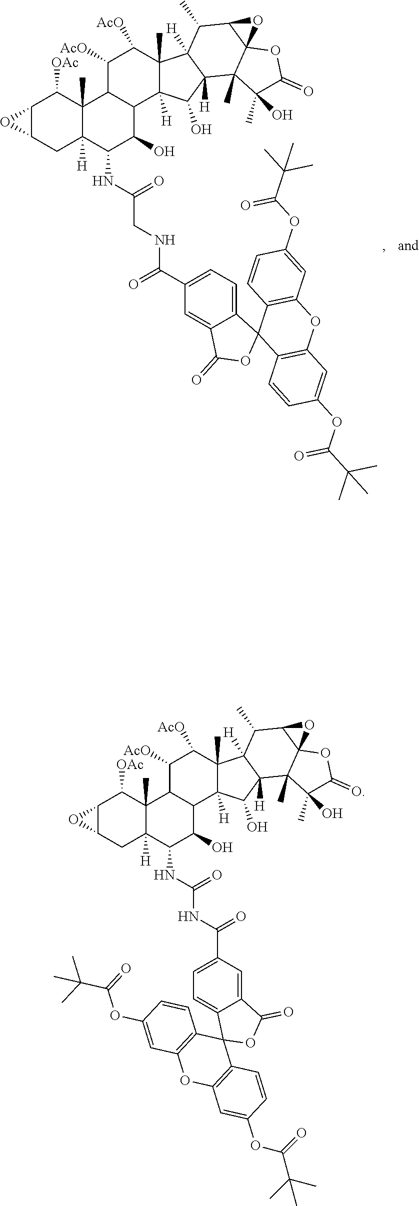

In a further aspect, the compound has a structure represented by a formula:

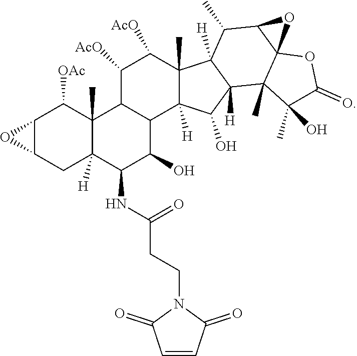 [SMILES: CC(=O)O[C@H]1C2C([C@@H](O)[C@@H](NC(=O)CCN3C(=O)C=CC3=O)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

In a further aspect, the compound has a structure represented by a formula:

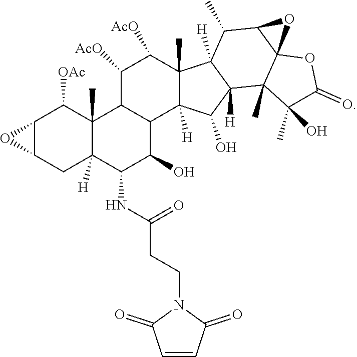 [SMILES: CC(=O)O[C@H]1C2C([C@@H](O)[C@H](NC(=O)CCN3C(=O)C=CC3=O)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

In a further aspect, the compound has a structure selected from:

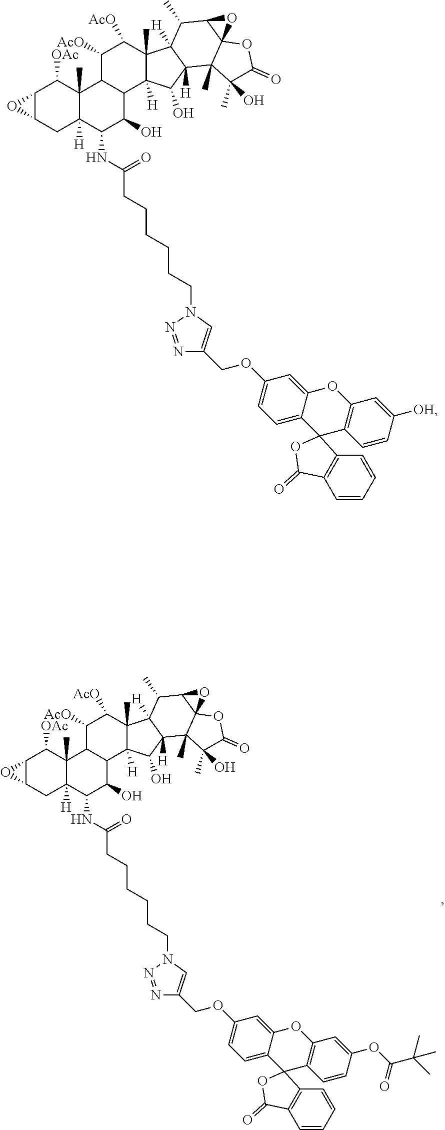 [SMILES: CC(=O)O[C@H]1C2C([C@@H](O)[C@H](NC(=O)CCCCCCn3cc(COc4ccc5c(c4)Oc4cc(O)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@H](NC(=O)CCCCCCn3cc(COc4ccc5c(c4)Oc4cc(OC(=O)C(C)(C)C)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; ","]

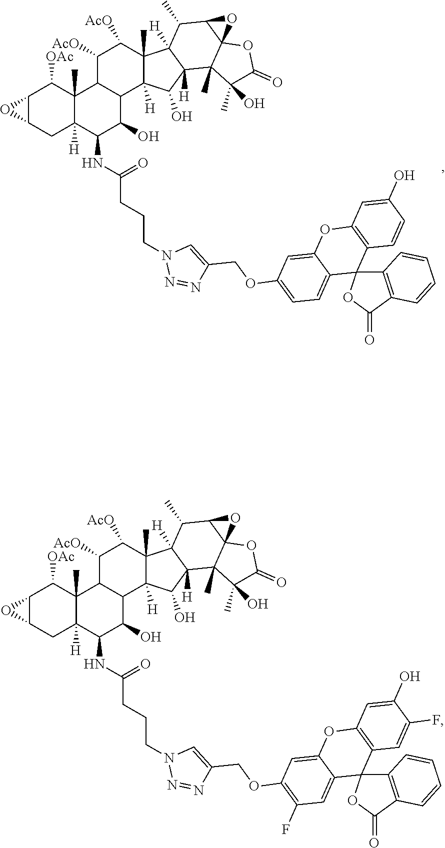 [SMILES: CC(=O)O[C@H]1C2C([C@@H](O)[C@@H](NC(=O)CCCn3cc(COc4ccc5c(c4)Oc4cc(O)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; "," ; CC(=O)O[C@H]1C2C([C@@H](O)[C@@H](NC(=O)CCCn3cc(COc4cc5c(cc4F)C4(OC(=O)c6ccccc64)c4cc(F)c(O)cc4O5)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

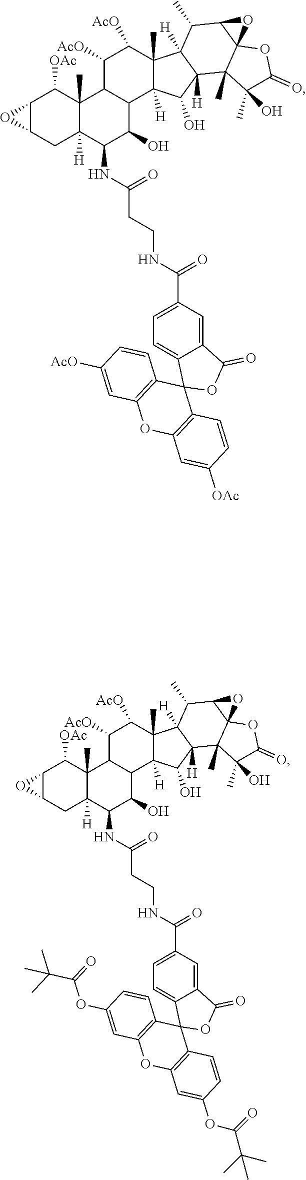

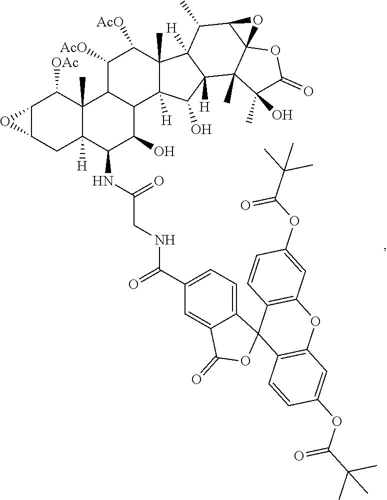 [SMILES: CC(=O)O[C@H]1C2C([C@@H](O)[C@@H](NC(=O)CNC(=O)c3ccc4c(c3)C(=O)OC43c4ccc(OC(=O)C(C)(C)C)cc4Oc4cc(OC(=O)C(C)(C)C)ccc43)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; ","]

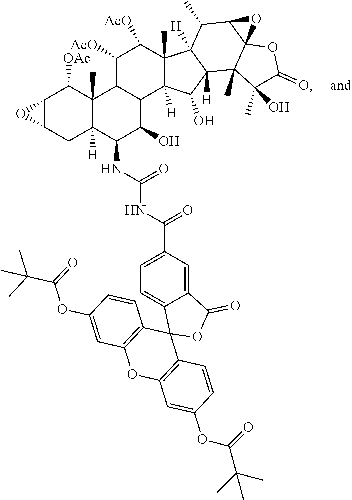 [SMILES: CC(=O)O[C@H]1C2C([C@@H](O)[C@@H](NC(=O)NC(=O)c3ccc4c(c3)C(=O)OC43c4ccc(OC(=O)C(C)(C)C)cc4Oc4cc(OC(=O)C(C)(C)C)ccc43)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; "and"]

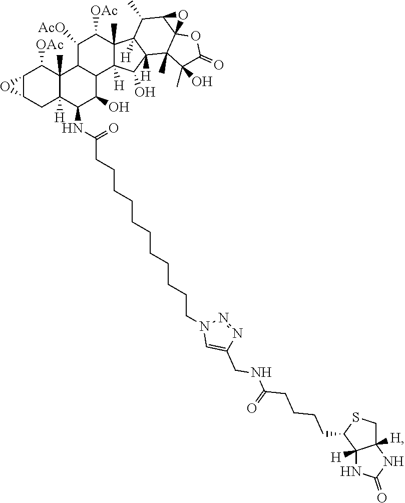

In a further aspect, the compound has a structure selected from:

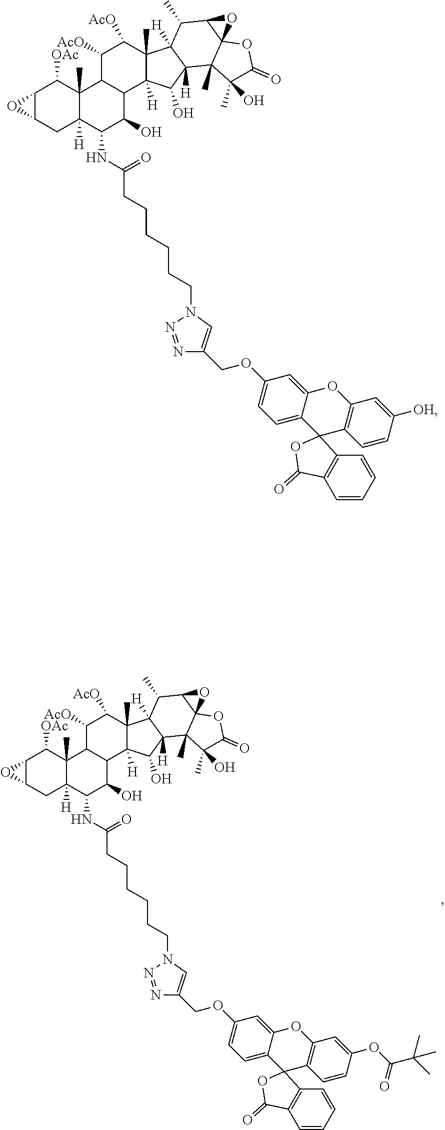 [SMILES: CC(=O)O[C@H]1C2C([C@@H](O)[C@H](NC(=O)CCCCCCn3cc(COc4ccc5c(c4)Oc4cc(O)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@H](NC(=O)CCCCCCn3cc(COc4ccc5c(c4)Oc4cc(OC(=O)C(C)(C)C)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; ","]

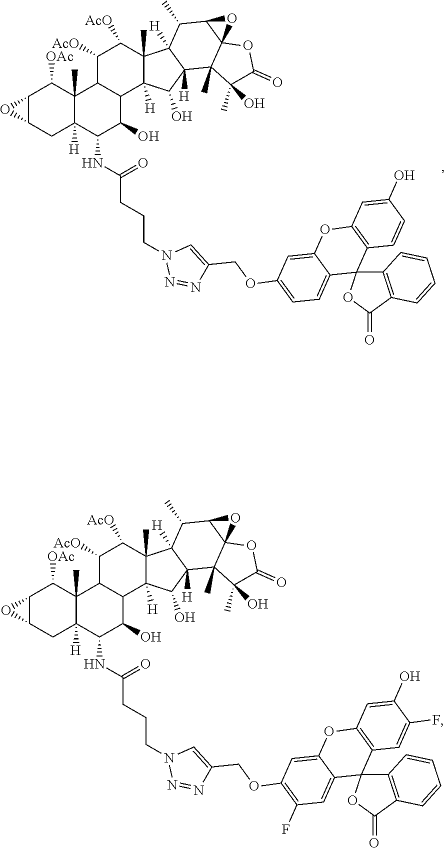 [SMILES: CC(=O)O[C@H]1C2C([C@@H](O)[C@H](NC(=O)CCCn3cc(COc4ccc5c(c4)Oc4cc(O)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; "," ; CC(=O)O[C@H]1C2C([C@@H](O)[C@H](NC(=O)CCCn3cc(COc4cc5c(cc4F)C4(OC(=O)c6ccccc64)c4cc(F)c(O)cc4O5)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

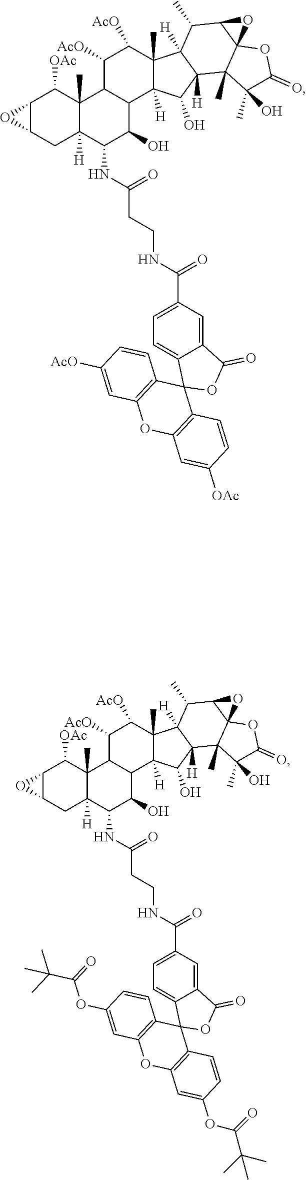

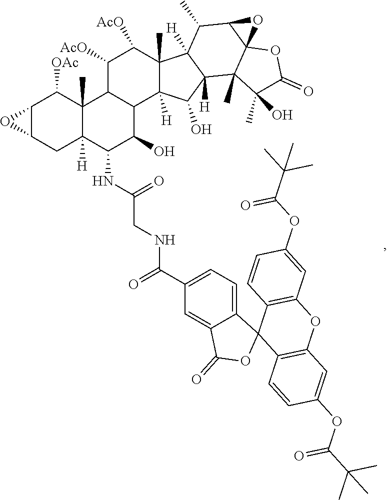 [SMILES: CC(=O)O[C@H]1C2C([C@@H](O)[C@H](NC(=O)CNC(=O)c3ccc4c(c3)C(=O)OC43c4ccc(OC(=O)C(C)(C)C)cc4Oc4cc(OC(=O)C(C)(C)C)ccc43)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; ","]

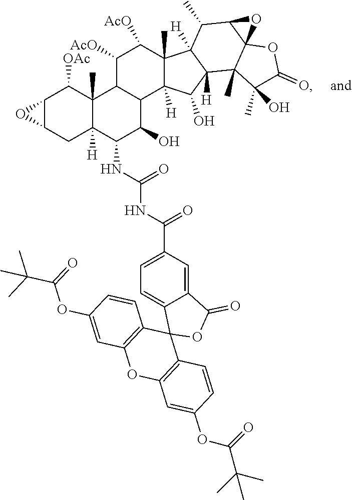 [SMILES: CC(=O)O[C@H]1C2C([C@@H](O)[C@H](NC(=O)NC(=O)c3ccc4c(c3)C(=O)OC43c4ccc(OC(=O)C(C)(C)C)cc4Oc4cc(OC(=O)C(C)(C)C)ccc43)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; "and"]

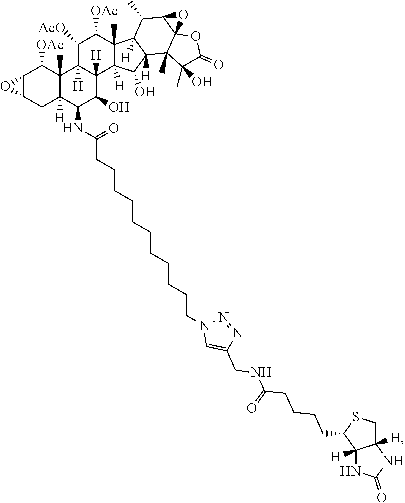

In a further aspect, the compound has a structure selected from:

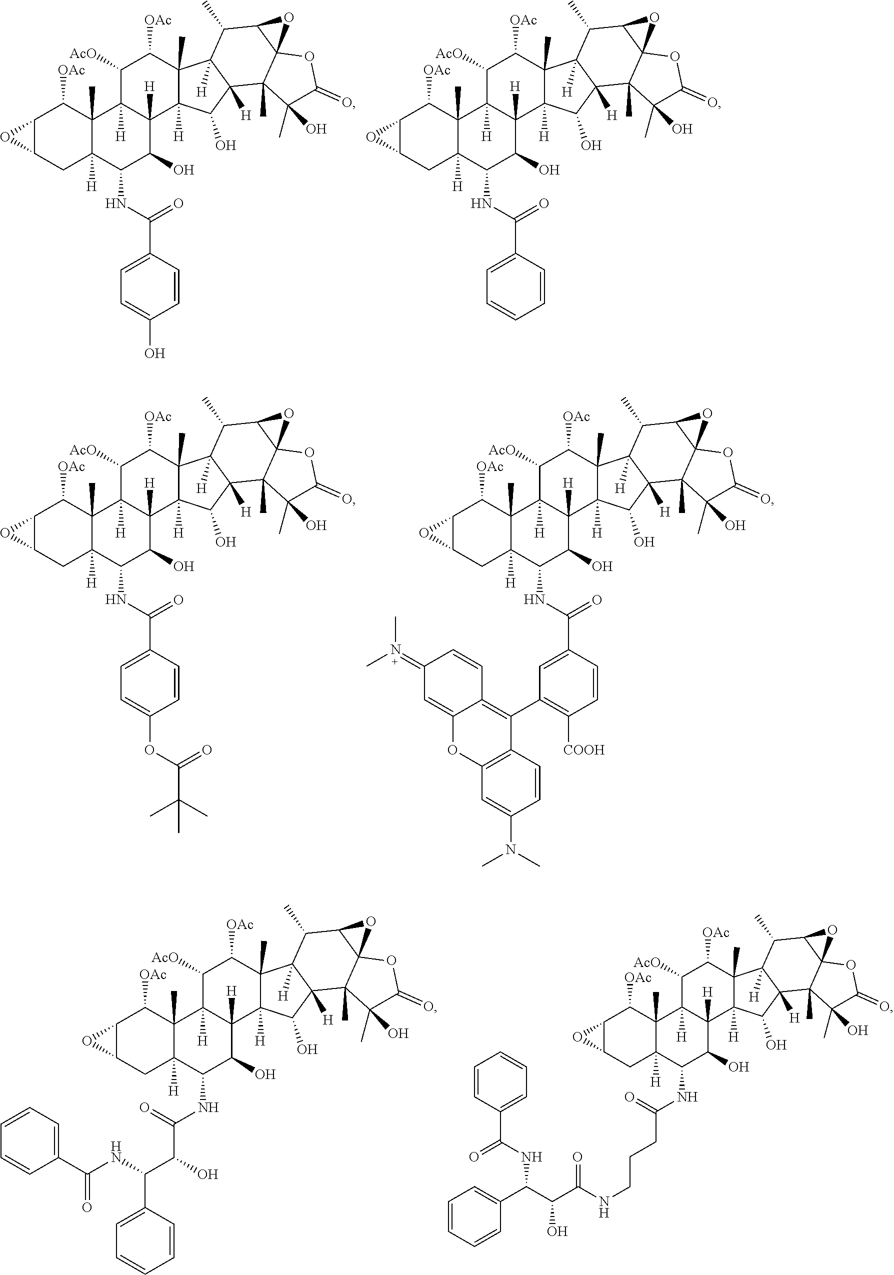

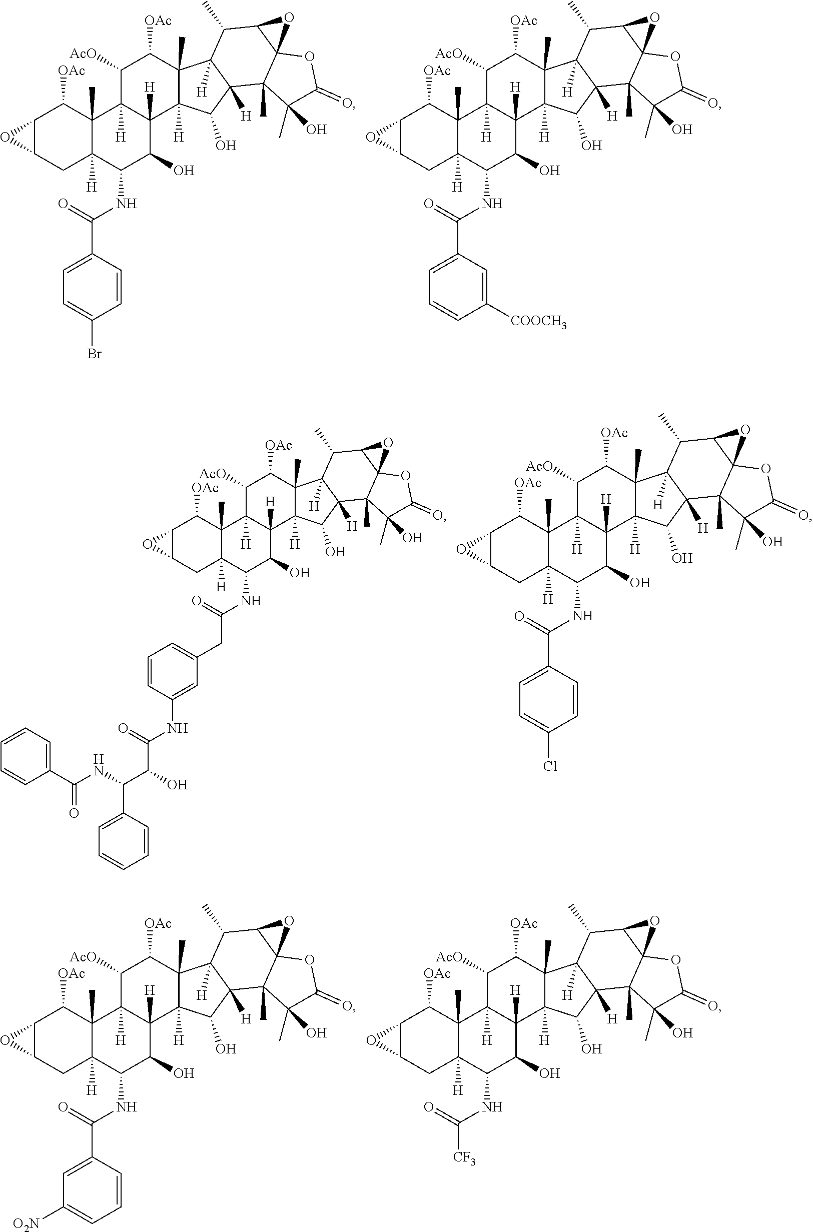

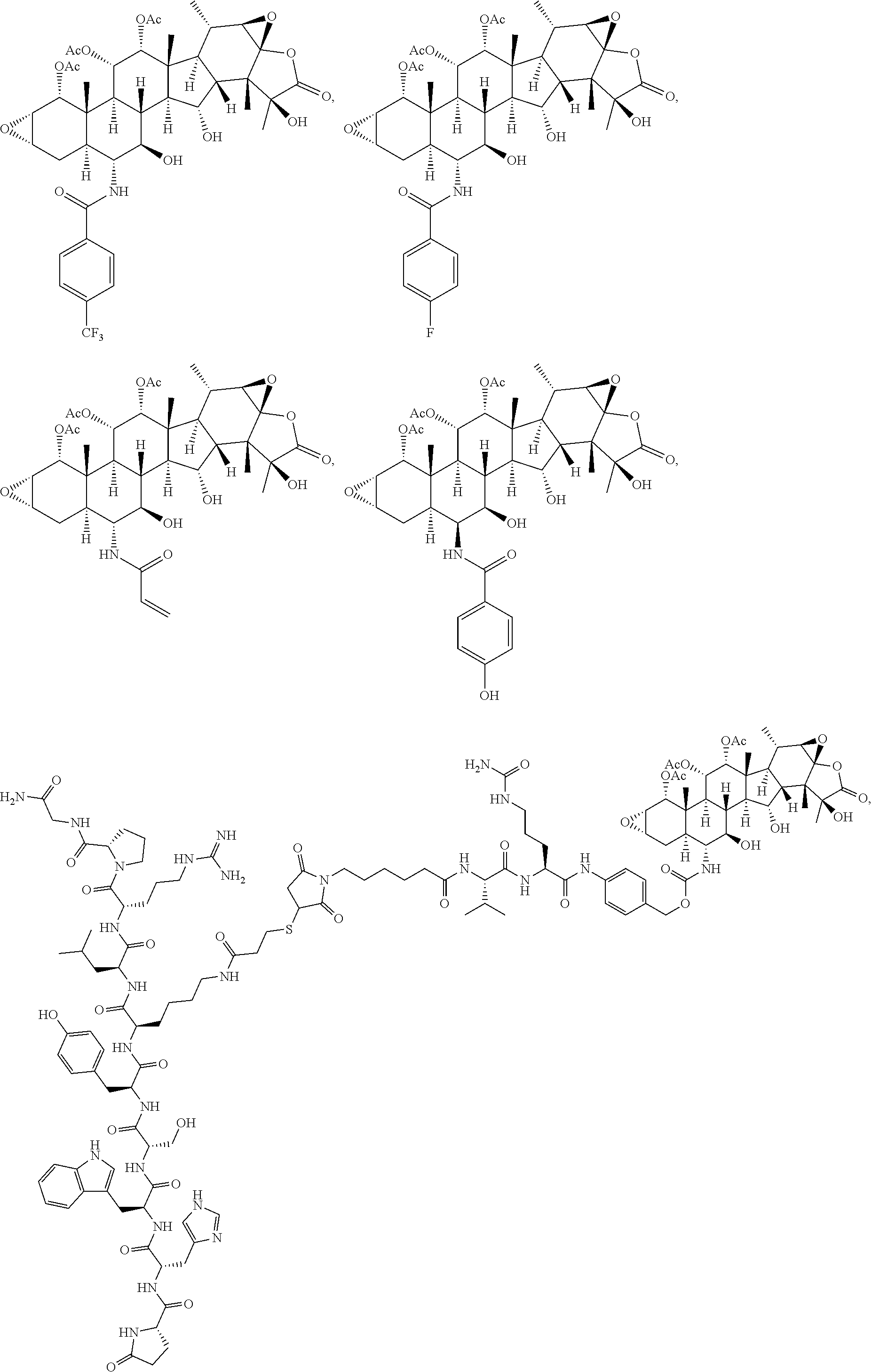

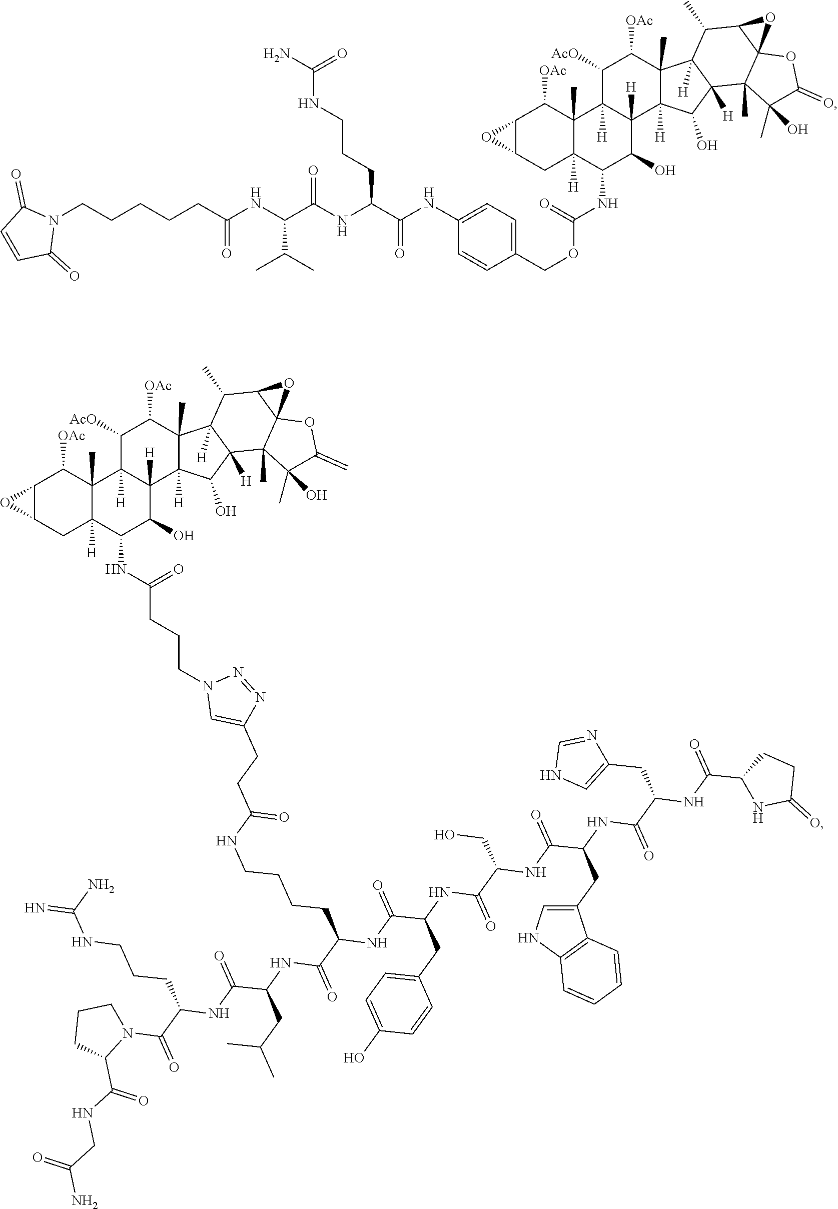

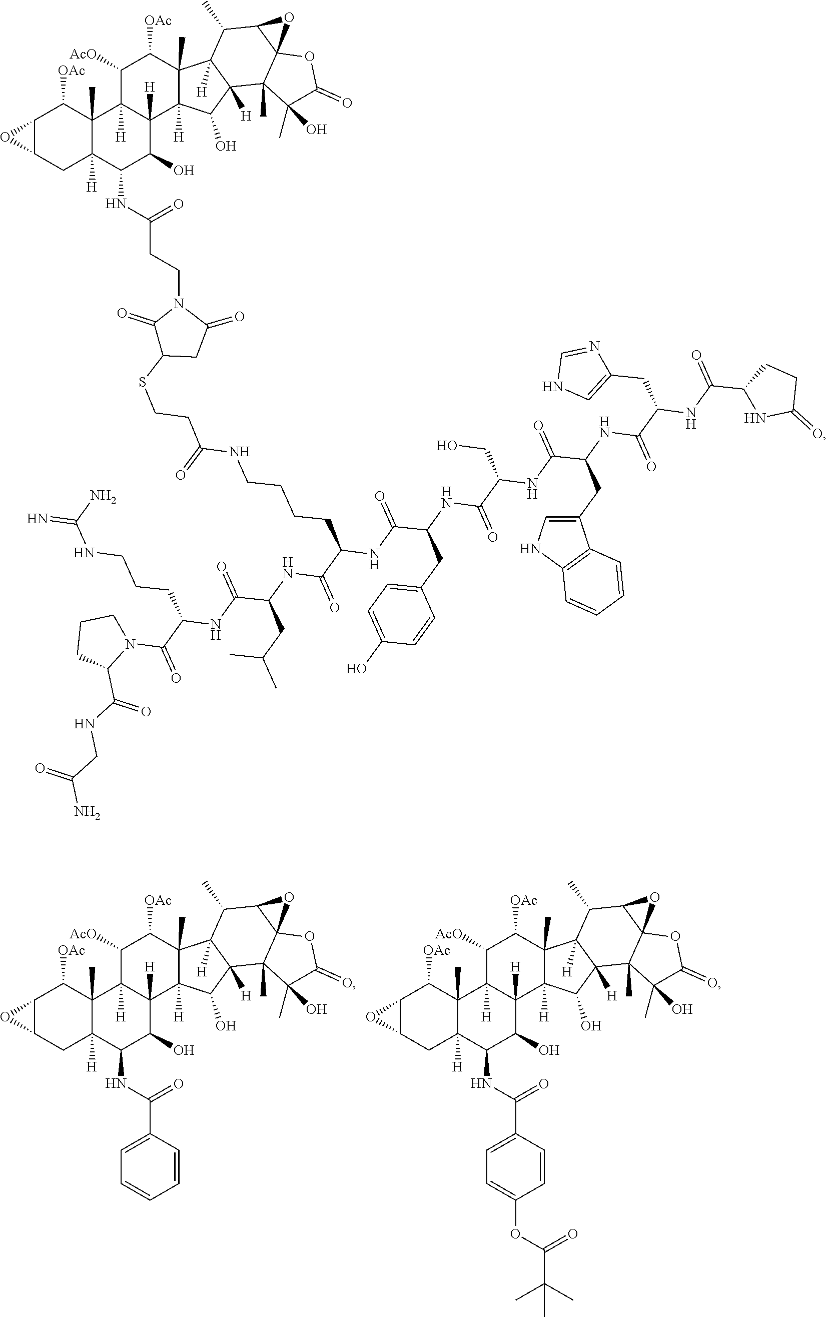

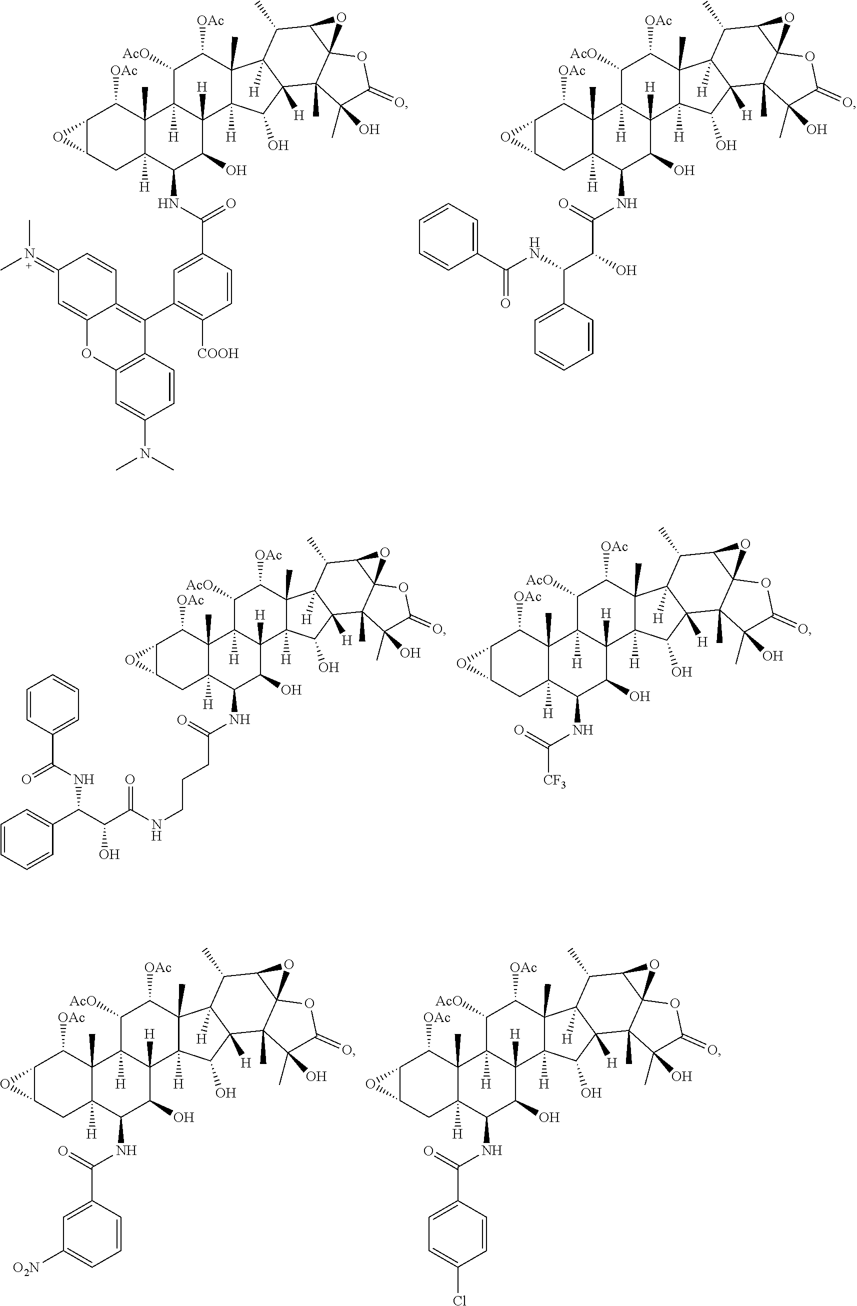

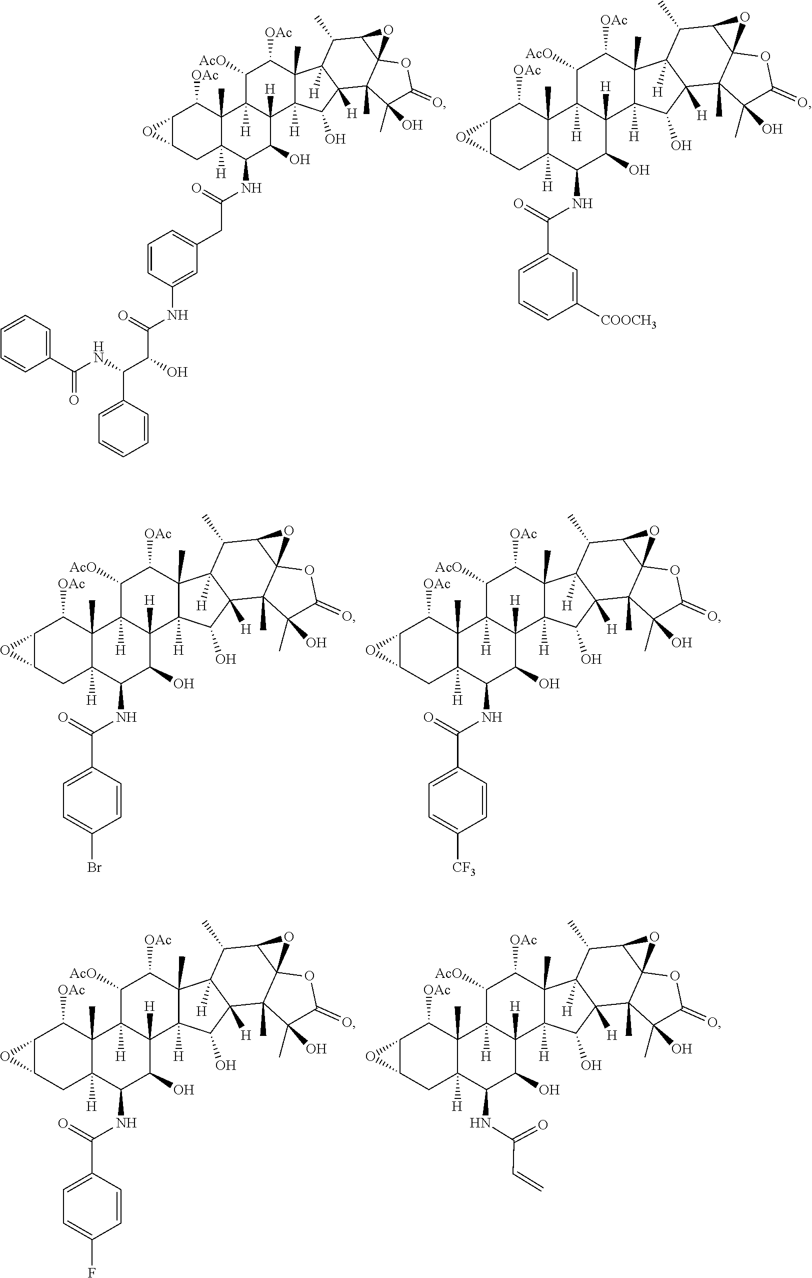

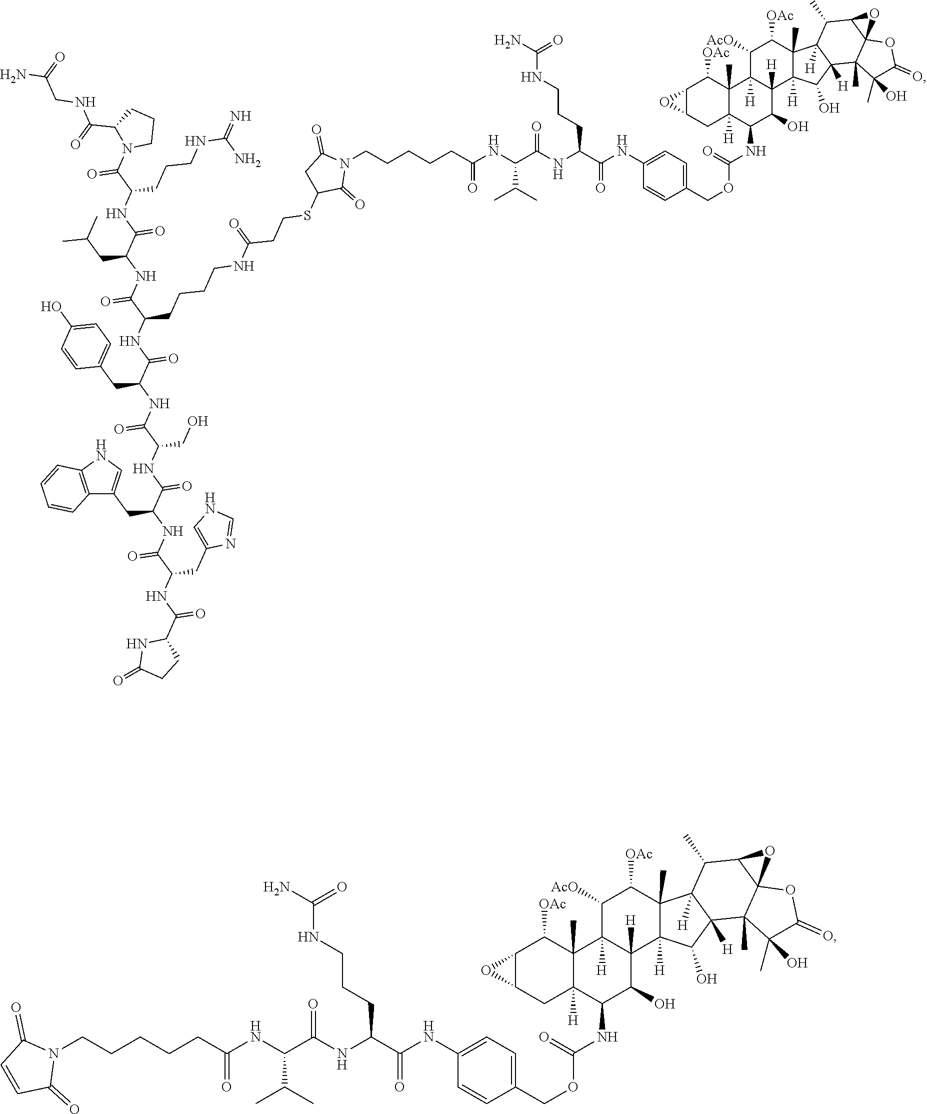

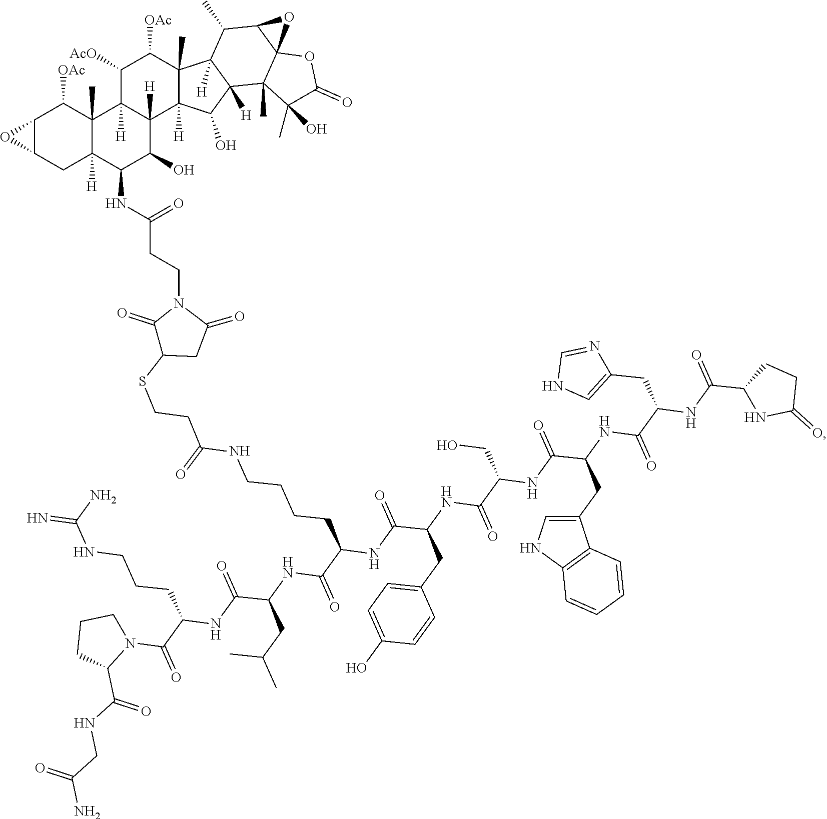

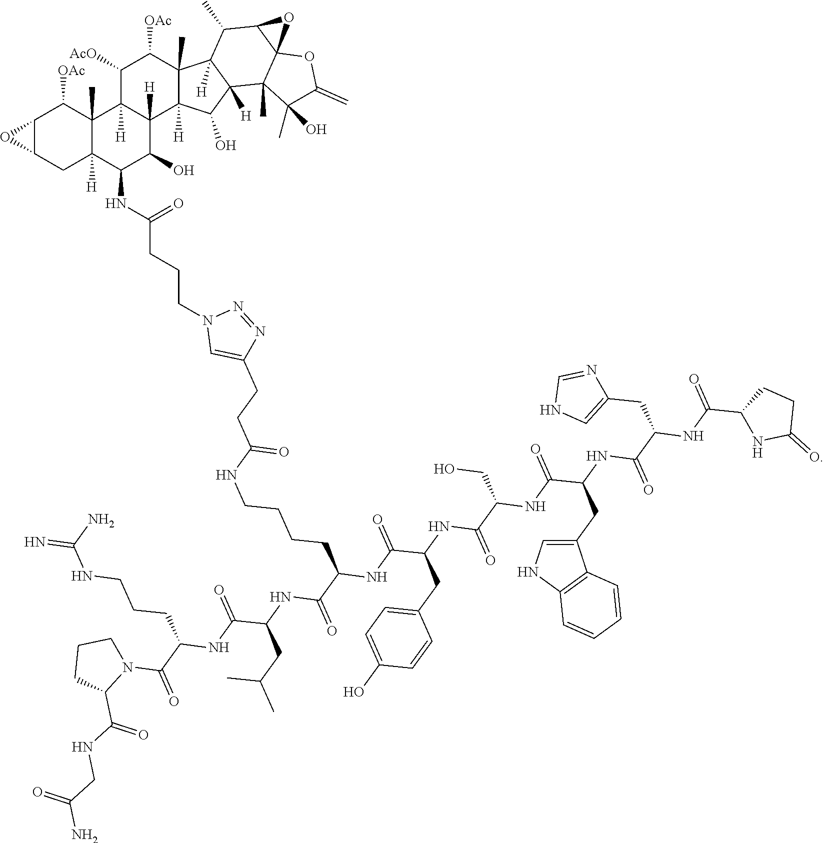

In a further aspect, the compound has a structure selected from:

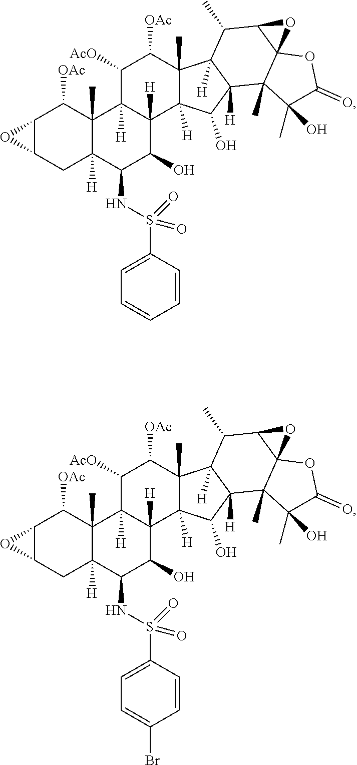

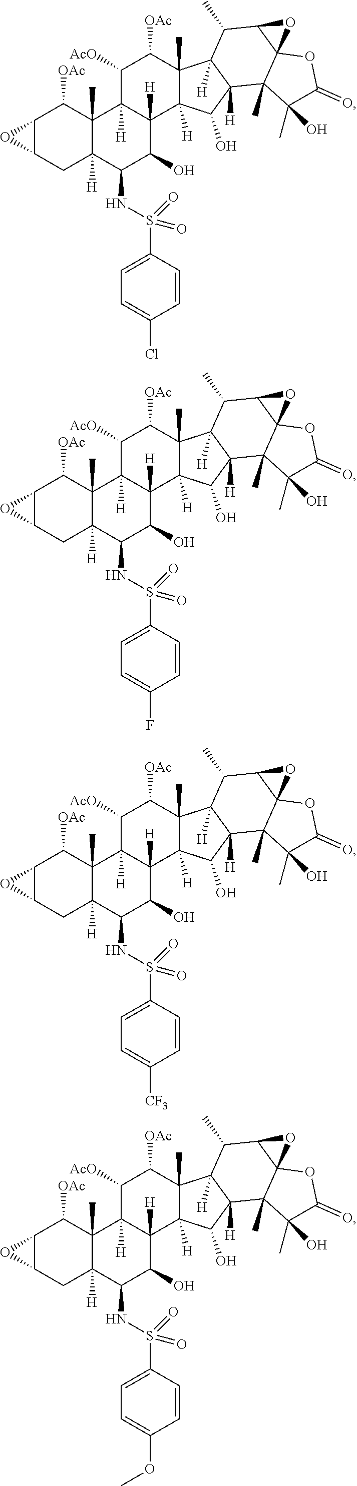

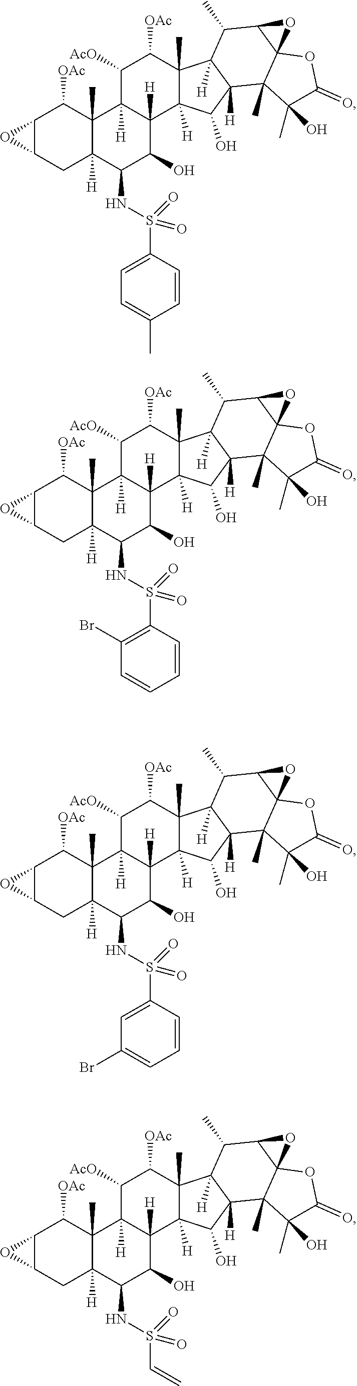

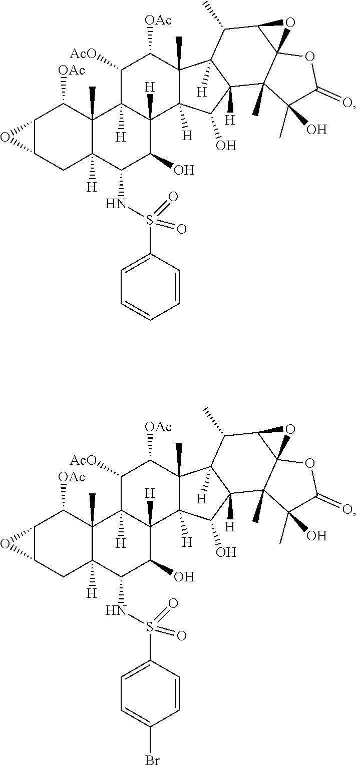

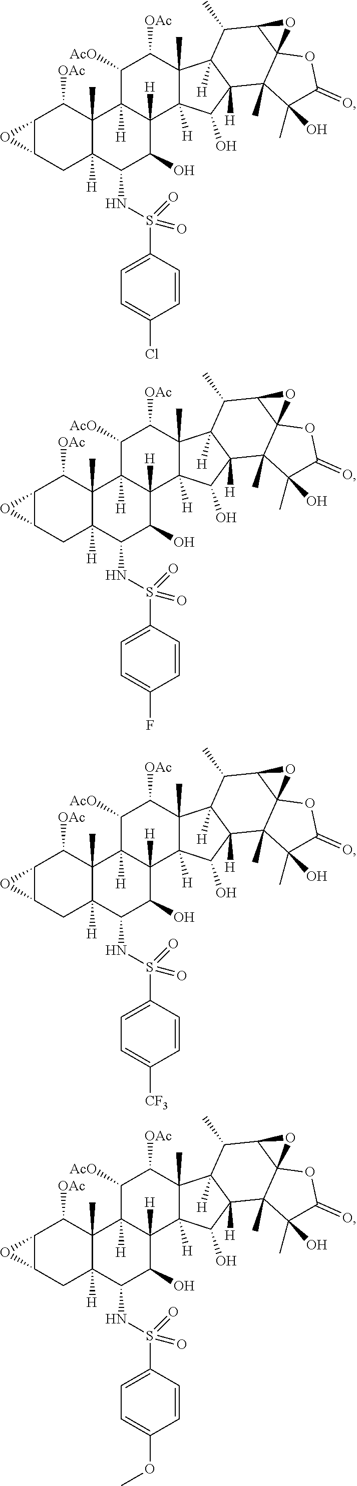

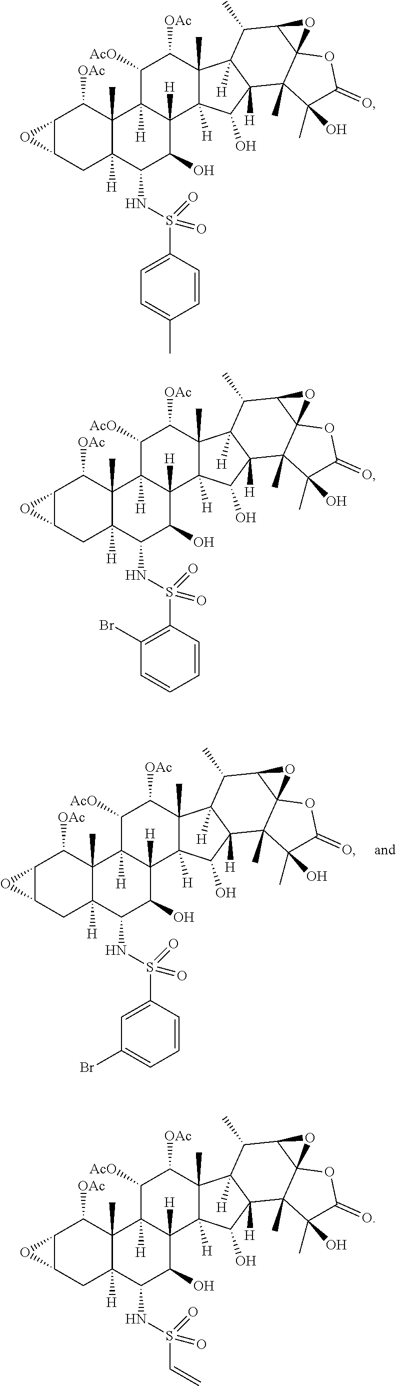

In a further aspect, the compound has a structure selected from:

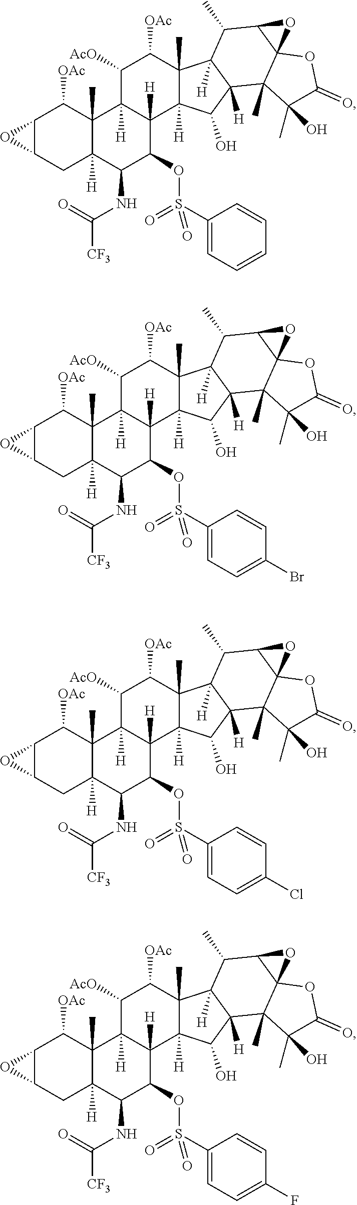

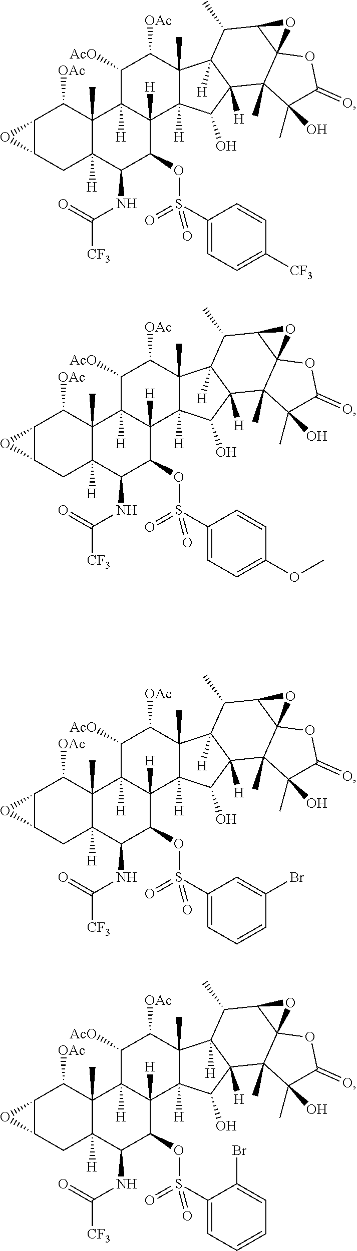

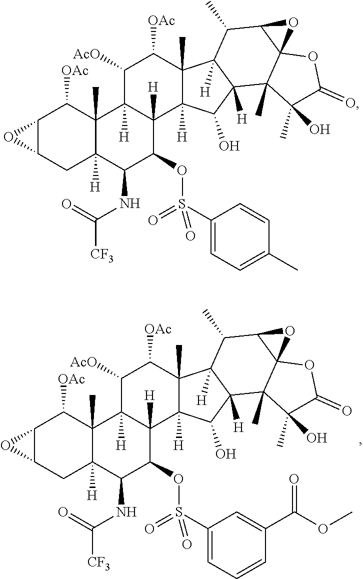

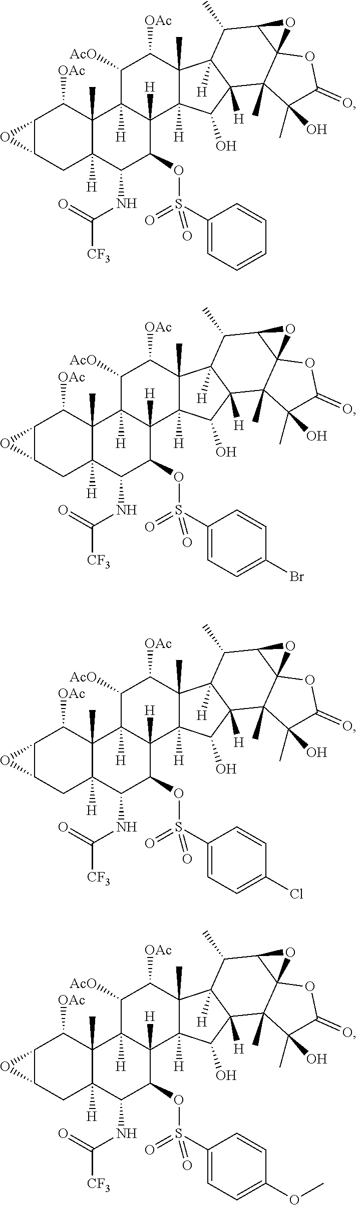

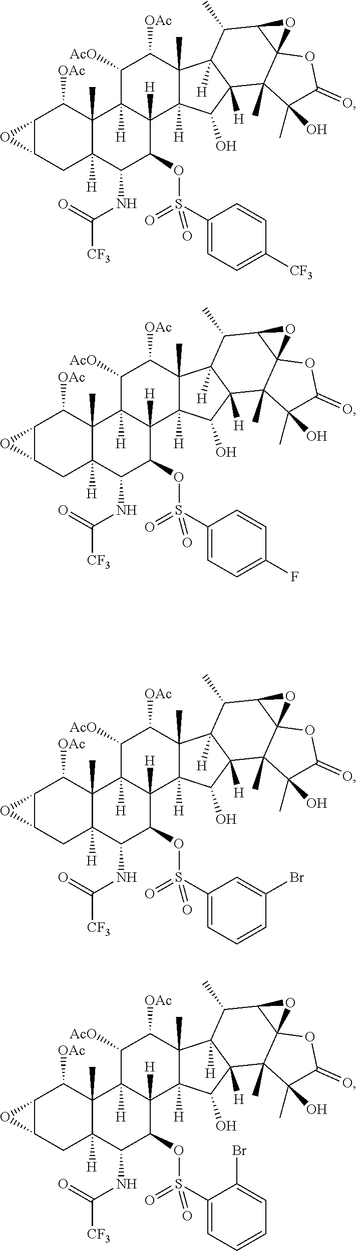

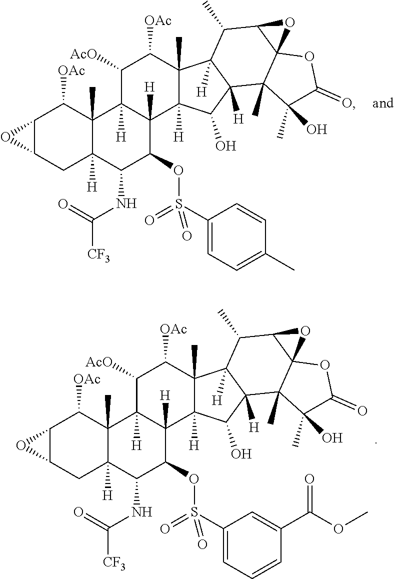

In a further aspect, R²⁰ is methyl and wherein R²¹ is hydrogen.

In a further aspect, L is selected from —NR⁶¹C(O)—, —C(O)NR⁶¹—, —NR⁶¹C(S)NR⁶²—, —SCH₂C(O)—, —C(O)SCH₂—,

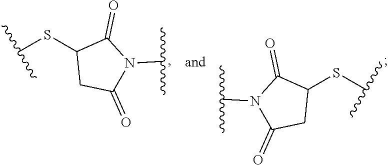 [SMILES: *SC1CC(=O)N(*)C1=O ; *SC1CC(=O)N(*)C1=O]

and each of R⁶¹ and R⁶², when present, is independently selected from hydrogen and C1-C12 alkyl.

In a further aspect, each occurrence of - - - - - - is a single covalent bond.

In a further aspect, the occurrence of - - - - - - at C-2/C-3 is a double covalent bond. In a still further aspect, the occurrence of - - - - - - at C-2/C-3 is a single covalent bond.

In a further aspect, the occurrence of - - - - - - at C-5/C-6 is a double covalent bond. In a still further aspect, the occurrence of - - - - - - at C-5/C-6 is a single covalent bond.

In a further aspect, the occurrence of - - - - - - at C-7/C-8 is a double covalent bond. In a still further aspect, the occurrence of - - - - - - at C-7/C-8 is a single covalent bond.

In a further aspect, the occurrence of - - - - - - at C-1l/C-12 is a double covalent bond. In a still further aspect, the occurrence of - - - - - - at C-11/C-12 is a single covalent bond.

In a further aspect, the occurrence of - - - - - - at C-22/C-23 is a double covalent bond. In a still further aspect, the occurrence of - - - - - - at C-22/C-23 is a single covalent bond.

a. L Groups

In one aspect, L is a linker. Examples of linkers include, but are not limited to, polyethers, small aryl groups (e.g., 1,4-linked benzyl), disulfides, ethers, thioethers, esters, sulfonamides, dipeptides, maleimidocaproyl, hydrazines, hydrazones, acylhydrazines, acylhydrazones, and 1,2,3-triazoles. In various aspects, the linker is a chemical linker known in relation to antibody-drug conjugates (ADCs). Thus, in a further aspect, the linker connects the Z group (i.e., the antibody, the antibody fragment, the vitamin, the hormone, the carbohydrate, the molecular ligand, the aptamer, the non-antibody protein, the peptide, the nucleic acid, the fluorophore, or the drug) to the compound (e.g., the central core of the compound). Desirable qualities of the linker include, but are not limited to, providing stability prior to entering a target cell, providing efficient payload release once inside the target cell (e.g., via endosomoal or lysosomal degradation), and compatibility with the Z group and the compound.

Examples of compounds having a variety of different linkers are shown below. Such examples are not meant to be limiting.

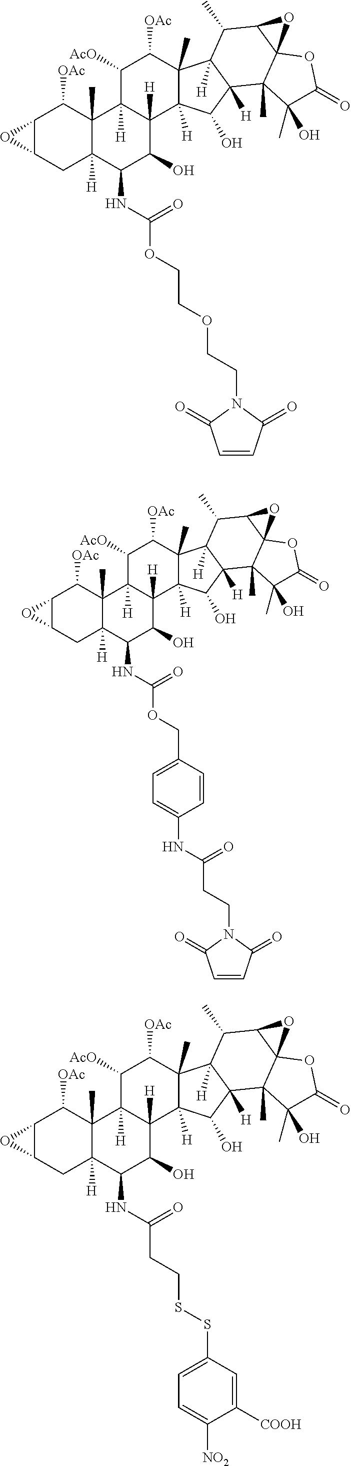

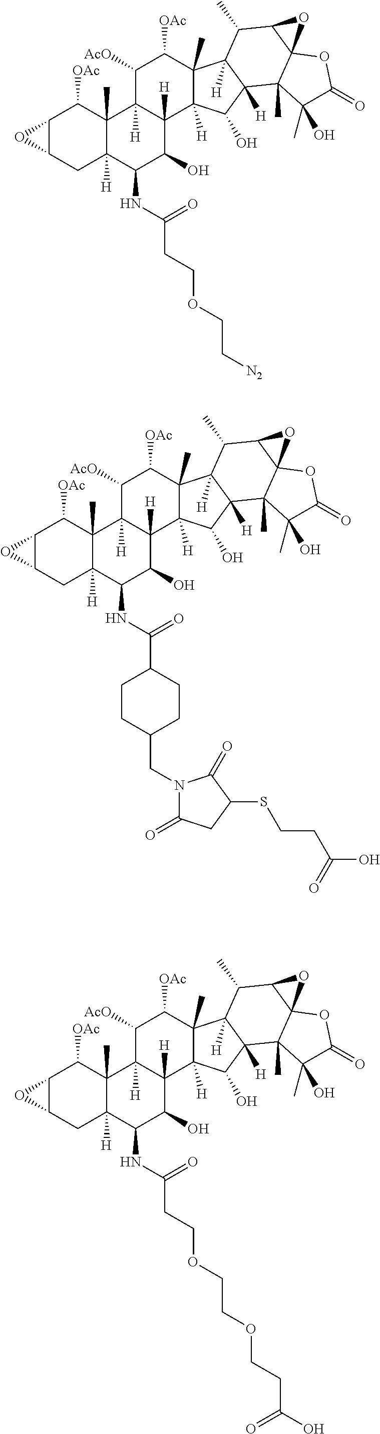

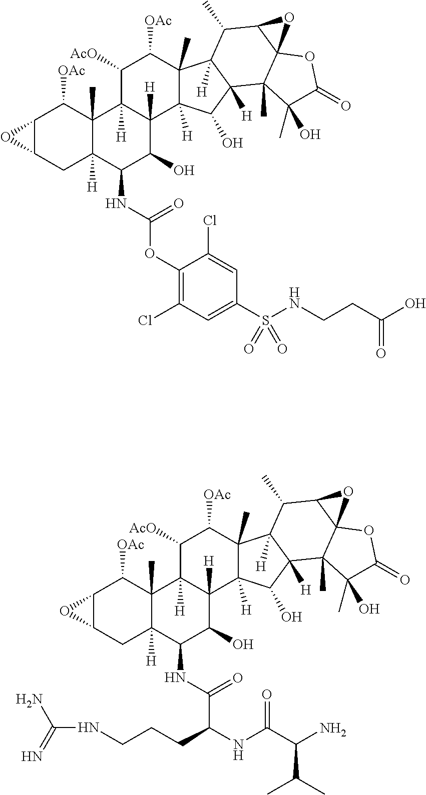

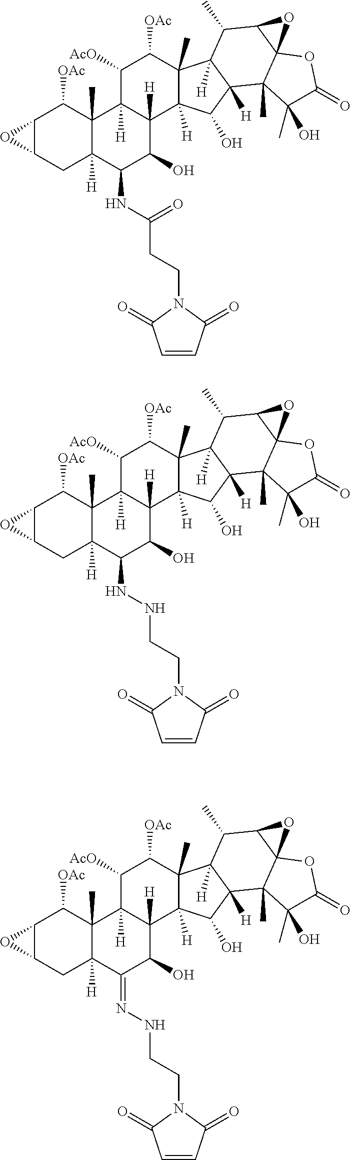

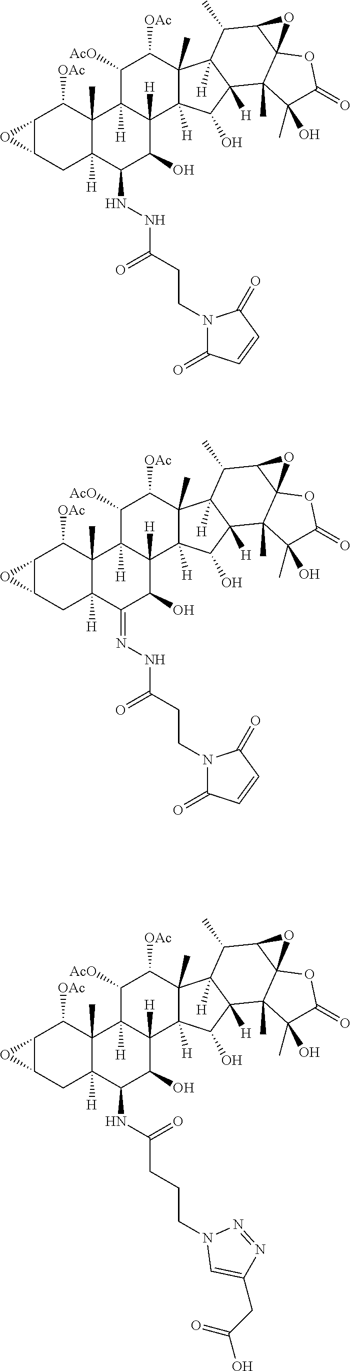

In a further aspect, the linker is cleavable (i.e., the linker relies on the physiological environment and releases a payload via hydrolyzation or proteoloysis in the target cells). Examples of cleavable linkers include, but are not limited to, chemically labile linkers (i.e., acid cleavable linkers such as hydrazines and silyl ethers and reducible linkers) and enzyme cleavable linkers (i.e., linkers that rely on the presence of hydrolytic enzymes in the cell). Enzyme cleavable linkers include, but are not limited to, peptide-based linkers (e.g. valine-citrulline) dipeptide linkers and phenylalanine-lysine dipeptide linkers) and beta-glucuronide linkers. In various aspects, a cleavable linker is broken down in the cells to release a compound.

In a further aspect, the linker is non-cleavable (i.e., the linker cannot be broken down outside a target cell). Advantages of a non-cleavable linker include, but are not limited to, increased plasma stability and larger therapeutic windows. In various aspects, a non-cleavable linker remains attached to a compound in cells.

In a further aspect, the linker is a tertiary amine linker (e.g., monomethyl auristatin E).

In various aspects, the linker is humanized IgG4, hP67/6 hydrazone; humanized IgG4, G5/44 hydrazone; milatuzamab hydrazine; humanized IgG1, huC242 disulfide; humanized IgG1, DS6 disulfide; anti-CD138 chimeric IgG4 disulfide, anti-mesothelin fully human IgG1 disulfide; anti-integrin, IgG1 disulfide; anti-cripto IgG1 disulfide; hu134, humanized IgG1 disulfide; anti-CD30 dipeptide; anti-CR011 dipeptide; anti-CD70 dipeptide; anti-nectin fully human IgG dipeptide; anti-PSMA fully human IgG1 dipeptide; trastuzumab, humanized IgG1 thioether; Ch1gG1 thioether; K7153A humanized IgG1 thioether; or anti-EGFRvIII fully human IgG1 thioether.

In a further aspect, the linker is a disulfide linker. Examples of disulfide linkers include, but are not limited to:

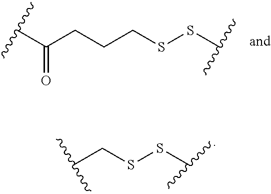 [SMILES: *SSCCCC(*)=O ; *CSS*]

In a further aspect, the linker is a thioether linker. An example of a thioether linker is, but is not limited to:

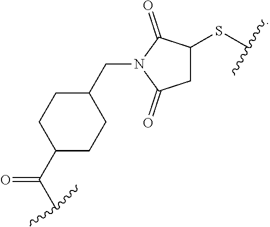 [SMILES: *SC1CC(=O)N(CC2CCC(C(*)=O)CC2)C1=O]

In a further aspect, the linker is a dipeptide linker. An example of a dipeptide linker is, but is not limited to:

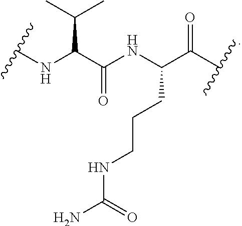

In a further aspect, the linker is a maleimidocaproyl linker. An example of a maleimidocaproyl linker is, but is not limited to:

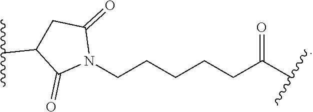 [SMILES: *C(=O)CCCCCN1C(=O)CC(*)C1=O]

In a further aspect, the linker is a hydrazone. Examples of hydrazone linkers include, but are not limited to:

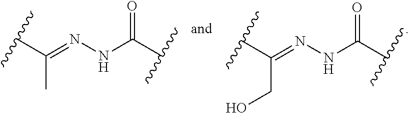

In a further aspect, L is selected from —NR⁶¹C(O)—, —C(O)NR⁶¹—, —NR⁶¹C(S)NR⁶²—, —SCH₂C(O)—, —C(O)SCH₂—,

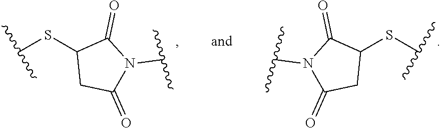 [SMILES: *SC1CC(=O)N(*)C1=O ; *SC1CC(=O)N(*)C1=O]

In a further aspect, L is selected from

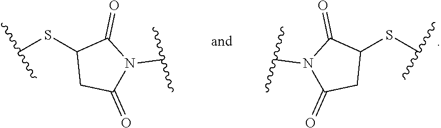 [SMILES: *SC1CC(=O)N(*)C1=O ; *SC1CC(=O)N(*)C1=O]

In a still further aspect, L is

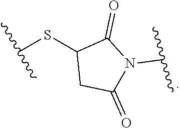 [SMILES: *SC1CC(=O)N(*)C1=O]

In yet a further aspect, L is

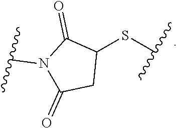 [SMILES: *SC1CC(=O)N(*)C1=O]

In a further aspect, L is selected from —NR⁶¹C(O)—, —C(O)NR⁶¹—, —NR⁶¹C(S)NR⁶², —SCH₂C(O)—, and —C(O)SCH₂—. In a still further aspect, L is selected from —NR⁶¹C(O)— and —C(O)NR⁶¹—. In yet a further aspect, L is —NR⁶¹C(O)—. In an even further aspect, L is —C(O)NR⁶¹—.

In a further aspect, L is selected from —NR⁶¹C(S)NR⁶²—, —SCH₂C(O)—, and —C(O)SCH₂—. In a still further aspect, L is selected from —SCH₂C(O)— and —C(O)SCH₂—. In yet a further aspect, L is —NR⁶¹C(S)NR⁶²—. In an even further aspect, L is —SCH₂C(O)—. In a still further aspect, L is —C(O)SCH₂—.

b. X Groups

In one aspect, X is selected from O, NR^{x}, and C(R^{x})₂. In a further aspect, X is selected from O and NR^{x}. In a still further aspect, X is selected from O and C(R^{x})₂. In yet a further aspect, X is selected from NR^{x} and C(R^{x})₂. In an even further aspect, X is O. In a still further aspect, X is NR^{x}. In yet a further aspect, X is C(R^{x})₂.

c. Z Groups

In one aspect, Z is selected from an antibody, an antibody fragment, a vitamin, a hormone, a carbohydrate, a molecular ligand, an aptamer, a non-antibody protein, a peptide, a nucleic acid, a fluorophore, and a drug.

In a further aspect, Z is an antibody. As used herein, the term “antibody” means a protein made by plasma cells in response to an antigen that typically consist of four subunits including two heavy chains and two light chains. Examples of antibodies include, but are not limited to, bevacizumab, trastuzumab, rituximab, abciximab, adalimumab, alemtuzumab, basiliximab, belimumab, brentuximab vedotin, canakinumab, cetuximab, certolizumab pegol, daclizumab, denosumab, eculizumab, efalizumab, gemtuzumab, golimumab, ibritumomab tiuxetan, infliximab, ipilimumab, muromnonab-CD3, natalizumab, ofatumumab, omalizumab, palivizumab, panitumumab, ranibizumab, raxibacumab, tocilizumab, tositumomab and ustekinumab. Other examples of antibodies include, but are not limited to, 3F8, abagovomab, abatacept, acz885, adecatumumab, afelimomab, aflibercept, afutuzumab, alacizumab, altumomab, anatumomab, anrukinzumab, apolizumab, arcitumomab, aselizumab, atlizumab, atorolimumab, bapineuzumab, bavituximab, bectumomab, belatacept, bertilimumab, besilesomab, biciromab, bivatuzumab, blinatumomab, cantuzumab, capromab, catumaxomab, cedelizumab, citatuzumab, cixutumumab, clenoliximab, cnto1275(=ustekinumab), cntol48(=golimumab), conatumumab, dacetuzumab, detumomab, dorlimomab, dorlixizumab, ecromeximab, edobacomab, edrecolomab, efungumab, elsilimomab, enlimomab, epitumomab, epratuzumab, erlizumab, ertumaxomab, etanercept, etaracizumab, exbivirumab, fanolesomab, faralimomab, felvizumab, figitumumab, fontolizumab, foravirumab, galiximab, gantenerumab, gavilimomab, gomiliximab, ibalizumab, igovomab, imciromab, inolimomab, inotuzumab ozogamicin, iratumumab, keliximab, labetuzumab, lebrilizunab, lemalesomab, lerdelimumab, lexatumumab, libivirurnab, lintuzunab, lucatumumab, lumiliximab, mapatumumab, maslimomab, matuzumab, mepolizumab, metelimumab, milatuzumab, minretumomab, mitumomab, morolimumab, motavizumab, myo-029, nacolomab, naptumomab, nebacumab, necitumumab, nerelimomab, nimotuzumab, nofetumomab, ocrelizumab, odulimomab, oportuzumab, oregovomab, otelixizumab, pagibaximab, panobacumab, pascolizumab, pemtumomab, pertuzumab, pexelizumab, pintumomab, priliximab, pritumumab, pro-140, rafivirumab, ramucirumab, regavirumab, reslizumab, rilonacept, robatumumab, rovelizumab, rozrolimupab, ruplizumab, satumomab, sevirmmab, sibrotuzumab, siltuximab, siplizumab, solanezumab, sonepcizumab, sontuzumab, stamulumab, sulesomab, tacatuzumab, tadocizumab, talizumab, tanezumab, tapliturnomab, tefibazumab, telimomab, tenatumomab, teneliximab, teplizumab, tgn1412, ticilimumab (=tremelimumab), tigatuzumab, tnx-355 (=ibalizumab), tnx-650, tnx-901 (=talizumab), toralizumab, tremelimumab, tucotuzumab, tuvirumab, urtoxazumab, vapaliximab, vedolizumab, veltuzumab, vepalimomab, visilizumab, volociximab, votumumab, zalutumumab, zanolimumab, ziralimumab, and zolimomab.

In a further aspect, Z is an antibody fragment. As used herein, the term “antibody fragment” means a component derived from antigen-specific fragments of antibodies produced by recombinant processes. Three general types of fragments were observed, antigen-binding fragments (Fab), single chain variable fragments (scFv) and “third generation” (3G). Examples of antibody fragments include, but are not limited to, anti-HER2 scFv, Fv, Fab, Fab′, F(ab′)₂, Fab′-SH, and scFv.

In a further aspect, Z is a vitamin. As used herein, the term “vitamin” means any of a group of organic compounds that are essential for normal growth and nutrition and are required in small quantities in the diet because they cannot be synthesized by the body. Examples of vitamins include, but are not limited to, vitamin A, vitamin BI, vitamin B2, vitamin B6, vitamin K, vitamin C, vitamin D, niacin, biotin, pantothenic acid, folic acid, and vitamin B12.

In a further aspect, Z is a hormone. As used herein, the term “hormone” means a chemical substance produced in the body that controls and regulates the activity of certain cells or organs. Examples of hormones include, but are not limited to, estrogen, testosterone, insulin, androgen, progestogen, corticosteroids, growth hormone, androgens, melatonin, throxine, eicosanoids, adrenaline, glucagen, and steroids.

In a further aspect, Z is a carbohydrate. As used herein, the term “carbohydrate” means a naturally occurring organic compound that occurs in foods and living tissues. Examples of carbohydrates include, but are not limited to, sugars such as, for example, sucrose, glucose, fructose, maltose, xylitol, trehalose, galactose, dextrates, and maltodextrins; starches such as, for example, corn, wheat, potato, tapioca, barley, arrowroot, and rice; and celluloses such as, for example, methylcellulose, ethyl cellulose, cellulose acetate, cellulose acetate-butyrate, cellulose acetate-propionate, and cellulose propionate.

In a further aspect, Z is a molecular ligand, although it is noted that other possible options of Z may include some molecular ligands, as well. As used herein, the term “molecular ligand” means a small molecule that transmits signals in-between or within cells. Examples of molecular ligands include, but are not limited to, epidermal growth factor, angiopoietin, bone morphogenetic proteins, insulin like growth factor, adenosine triphosphate, and nicotinamide-adenine-dinucleotide, Flavin mononucleotide.

In a further aspect, Z is an aptamer. As used herein, the term “aptamer” means an oligonucleotide or peptide molecule that binds to a specific target molecule, Examples of aptamers include, but are not limited to, EpCAM aptamer, nucleic acid aptamers (e.g., DNA aptamers and RNA aptamers) and peptide aptamers.

In a further aspect, Z is a non-antibody protein. As used herein, the term “non-antibody protein” means a large molecule composed of one or more chains of amino acids in a specific order that is not an antibody as defined herein above. Examples of non-antibody proteins include, but are not limited to, albumin, insulin, receptors, actin, and tubulin.

In a further aspect, Z is a peptide. As used herein, the term “peptide” means a molecule consisting of from about 2 to about 50 amino acids. Examples of peptides include, but are not limited to, somatostatin peptide, luteinizing hormone releasing hormone, fusion proteins, receptors, ligands of cell surface proteins, secreted proteins, and enzymes.

In a still further aspect, Z is a nucleic acid. As used herein, the term “nucleic acid” means a residue consisting of either one or two long chains of repeating units of a nitrogen base (i.e., a purine or a pyrimidine base) attached to a sugar phosphate. Examples of nucleic acids include, but are not limited to, E2 RNA, cyclic adenosine monophosphate, nucleoside triphosphates, Flavin adenine dinucleotide, and nicotinamide adenine dinucleotide phosphate.

In a further aspect, Z is a fluorophore. Examples of fluorophores include, but are not limited to, fluorescein, Oregon green, rhoadmine, eosin, Texas red, indocarbocyanine, oxacarbocyanine, thiacarbocyanine, merocyanine, a squaraine derivative, a naphthalene derivative (e.g., a dansyl or prodan derivative), a coumarin derivative, an oxadiazole derivative (e.g., pyridyloxazole, nitrobenzoxadiazole, benzoxadiazole), an anthracene derivative (e.g., an anthraquinone such as DRAQ5, DRAQ7, and CyTRAK Orange), cascade blue, Nile red, Nile blue, cresyl violate, oxazine 170, proflavin, acridine orange, acridine yellow, auramine, crystal violet, malachite green, prophin, phthalocyanine, and bilirubin.

In a further aspect, Z is a drug or a therapeutic agent. Examples of drugs or therapeutic agents include, but are not limited to, taxanes such as paclitaxel, monomethyl auristatin E, mertansine and calicheamicin.

In a further aspect, Z is selected from an antibody and an antibody fragment.

d. R^{x} Groups

In one aspect, each occurrence of R, when present, is independently selected from hydrogen and C1-C6 alkyl. In a further aspect, each occurrence of R^{x}, when present, is independently selected from hydrogen and C1-C4 alkyl. In a still further aspect, each occurrence of R^{x}, when present, is hydrogen.

In a further aspect, each occurrence of R^{x}, when present, is independently selected from hydrogen, methyl, ethyl, n-propyl, isopropyl, n-butyl, isobutyl, sec-butyl, and tert-butyl. In a still further aspect, each occurrence of R^{x}, when present, is independently selected from hydrogen, methyl, ethyl, n-propyl, and isopropyl. In yet a further aspect, each occurrence of R^{x}, when present, is independently selected from hydrogen, methyl, and ethyl. In an even further aspect, each occurrence of R^{x}, when present, is independently selected from hydrogen and ethyl. In a still further aspect, each occurrence of R^{x}, when present, is independently selected from hydrogen and methyl.

In a further aspect, each occurrence of R, when present, is independently selected from C1-C6 alkyl. In a still further aspect, each occurrence of R^{x}, when present, is independently selected from methyl, ethyl, n-propyl, isopropyl, n-butyl, isobutyl, sec-butyl, and tert-butyl. In yet a further aspect, each occurrence of R, when present, is independently selected from methyl, ethyl, n-propyl, and isopropyl. In an even further aspect, each occurrence of R^{x}, when present, is independently selected from methyl and ethyl. In a still further aspect, each occurrence of R^{x}, when present, is ethyl. In yet a further aspect, each occurrence of R^{x}, when present, is methyl.

e. R¹ and R^{1′} Groups

In one aspect. R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁷, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹ and R^{1′} is hydrogen. In a further aspect, each of R¹ and R^{1′} is hydrogen.

In one aspect, each of R¹ and R^{1′} together comprise ═O or ═NR³⁶. In a further aspect, each of R¹ and R^{1′} together comprise ═O. In a still further aspect, each of R¹ and R^{1′} together comprise ═NR³⁶.

In a further aspect, R¹ is selected from hydrogen, halogen, —OH, —CN, —NC, —NCO, —OCN, —NO₂, —ONO₂, —ONO, —NO, —N₃, —NH₂, —NH₃, —N═NR^{3′}, —NHOH, C1-C8 alkyl, C2-C8 alkenyl, C2-C8 alkynyl, C1-C8 hydroxy, C1-C8 alkoxy, C1-C8 thioalkyl, C1-C8 alkylthiol, C1-C8 aminoalkyl, C1-C8 alkylamino, (C1-C8)(C1-C8) dialkylamino, —OC(O)(C1-C8 alkyl), —OP(O)(OR³²)₂, —OSO₂R³³, —C(O)(C1-C8 alkyl), —CO₂R³⁴, —C(O)NR^{35a}R^{35b}, —(C1-C8 alkyl)C(O)NR^{35a}R^{35b}, —OC(O)NR^{35a}R^{35b}, —(C1-C8 alkyl)OC(O)NR^{35a}R^{35b}, Cy¹, Ar¹, (C1-C8 alkyl)Ar¹, and —OAr¹. In a still further aspect, R¹ is selected from hydrogen, halogen, —OH, —CN, —NC, —NCO, —OCN, —NO₂, —ONO₂, —ONO, —NO, —N₃, —NH₂, —NH₃, —N═NR³, —NHOH, C1-C4 alkyl, C2-C4 alkenyl, C2-C4 alkynyl, C1-C4 hydroxy, C1-C4 alkoxy, C1-C4 thioalkyl, C1-C4 alkylthiol, C1-C4 aminoalkyl, C1-C4 alkylamino, (C1-C4)(C1-C4) dialkylamino, —OC(O)(C1-C4 alkyl), —OP(O)(OR³²)₂, —OSO₂R³³, —C(O)(C1-C4 alkyl), —CO₂R³⁴, —C(O)NR^{35a}R^{35b}, —(C1-C8 alkyl)C(O)NR^{35a}R^{35b}, —OC(O)NR^{35a}R^{35b}, —(C1-C4 alkyl)OC(O)NR^{35a}R^{35b}, Cy¹, Ar¹, (C1-C4 alkyl)Ar¹, and —OAr¹, In yet a further aspect, R¹ is selected from hydrogen, —F, —Cl, —OH, —CN, —NC, —NCO, —OCN, —NO₂, —ONO₂, —ONO, —NO, —N₃, —NH₂, —NH₃, —N═NR³¹, —NHOH, methyl, ethyl, n-propyl, isopropyl, ethenyl, n-propenyl, isopropenyl, ethynyl, propynyl, —CH₂OH, —CH₂CH₂OH, —CH(CH₃)CH₂OH, —CH₂CF₂CH₂CH₂OH, —OCH₃, —OCH₂CH₃, —OCH(CH₃)₂, —OCH₂CH₂CH₃, —SCH₃, —SCH₂CH₃, —SCH(CH₃)₂, —SCH₂CH₂CH₃, —CH₂SH, —CH₂CH₂SH, —CH(CH₃)CH₂SH, —CH₂CH₂CH₂SH, —CH₂NH₂, —CH₂CH₂NH₂, —CH(CH₃)CH₂NH₂, —CH₂CH₂CH₂NH₂—, —NHCH₃, —NHCH₂CH₃, —NHCH(CH₃), —NHCH₂CH₂CH₃, —N(CH₃)₂, —N(CH₃)CH₂CH₃N, —N(CH₃)CH(CH3)₂, —N(CH₃)CH₂CH₂CH₃, —OC(O)CH₃, —OC(O)CH₂CH₃, —OC(O)CH(CH₃)₂, —OC(O)CH₂CH₂CH₃, —OP(O)(OR³²)₂, —OSO₂R³³, —C(O)CH₃, —C(O)CH₂CH₃, —C(O)CH(CH₃)₂, —C(O)CH₂CH₂CH₃, —CO₂R³⁴, —C(O)NR^{35a}R^{35b}, —CH₂C(O)NR^{35a}R^{35b}, —CH₂CH₂C(O)NR^{35a}R^{35b}, —CH(CH₃)CH₂C(O)NR^{35a}R^{35b}, —CH₂CH₂CH₂C(O)NR^{35a}R^{35b}, —OC(O)NR^{35a}R^{35b}, —CH₂OC(O)NR^{35a}R^{35b}, —CH₂CH₂OC(O)NR^{35a}R^{35b}, —CH(CH₃)CH₂OC(O)NR^{35a}R^{35b}, —CH₂CH₂CH₂OC(O)NR^{35a}R^{35b}, Cy¹, Ar¹, —CH₂Ar¹, —CH₂CH₂Ar¹, —CH(CH₃)CH₂Ar¹, —CH₂CH₂CH₂Ar¹, and —OAr¹. In an even further aspect. R¹ is selected from hydrogen, —F, —Cl, —OH, —CN, —NC, —NCO, —OCN, —NO₂, —ONO₂, —ONO, —NO, —N₃, —NH, —NH₃, —N═NR³¹, —NHOH, methyl, ethyl, ethenyl, ethynyl, —CH₂OH, —CH₂CH₂OH, —OCH₃, —OCH₂CH₃, —SCH₃, —SCH₂CH₃, —CH₂SH, —CH₂CH₂SH, —CH₂NH₂, —CH₂CH₂NH₂, —NHCH₃, —NHCH₂CH₃, —N(CH₃)₂, —N(CH₃)CH₂CH₃, —OC(O)CH₃, —OC(O)CH₂CH₃, —OP(O)(OR³)₂, —OSO₂R³³, —C(O)CH₃, —C(O)CH₂CH₃, —CO₂R³⁴, —C(O)NR^{35a}R^{35b}, —CH₂C(O)NR^{35a}R^{35b}, —CH₂CH₂C(O)NR^{35a}R^{35b}, —OC(O)NR^{35a}R^{35b}, —CH₂OC(O)NR^{35a}R^{35b}, —CH₂CH₂C(O)NR^{35a}R^{35b}, Cy¹, Ar¹, —CH₂Ar¹, —CH₂CH₂Ar¹, and —OAr¹. In a still further aspect, R¹ is selected from hydrogen, —F, —Cl, —OH, —CN, —NC, —NCO, —OCN, —NO₂, —ONO₂, —ONO, —NO, —N₃, —NH₂, —NH₃, —N═NR³¹, —NHOH, methyl, —CH₂OH, —OCH₃, —SCH₃, —CH₂SH, —CH₂NH₂, —NHCH₃, —N(CH₃)₂, —OC(O)CH₃, —OP(O)(OR³²)₂, —OSO₂R³³, —C(O)CH₃, —CO₂R³⁴, —C(O)NR^{35a}R^{35b}, —CH₂C(O)NR^{35a}R^{35b}, —OC(O)NR^{35a}R^{35b}, —CH₂OC(O)NR^{35a}R^{35b}, Cy¹, Ar¹, —CH₂Ar¹, and —OAr¹.

In various aspects, R¹ is selected from hydrogen, halogen. —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹. In a further aspect, R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C8 alkyl, C2-C8 alkenyl, C2-C8 alkynyl, —OC(O)(C1-C8 alkyl), —OP(O)(OR³²)₂, —OS₂R³³, —C(O)(C1-C8 alkyl), —CO₂R³⁴, —C(O)NR^{35a}R^{35b}, —(C1-C8 alkyl)C(O)NR^{35a}R^{35b}, —OC(O)NR^{35a}R^{35b}, —(C1-C8 alkyl)OC(O)NR^{35a}R^{35b}, Cy¹, Ar¹, (C1-C8 alkyl)Ar¹, and —OAr¹. In a still further aspect, R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C4 alkyl, C2-C4 alkenyl, C2-C4 alkynyl, —OC(O)(C1-C4 alkyl), —OP(O)(OR³²)₂, —OSO₂R³³, —C(O)(C1-C4 alkyl), —CO₂R³⁴, —C(O)NR^{35a}R^{35b}, (C1-C8 alkyl)C(O)NR^{35a}R^{35b}, —OC(O)NR^{35a}R^{35b}, —(C1-C4 alkyl)OC(O)NR^{35a}R^{35b}, Cy¹, Ar¹, (C1-C4 alkyl)Ar¹, and —OAr¹. In yet a further aspect, R¹ is selected from hydrogen, —F, —Cl, —OH, —CN, —NC, —NCO, —OCN, —NO₂, —ONO₂, —ONO, —NO, —N₃, —NH₂, —NH₃, —N═NR³¹, —NHOH, methyl, ethyl, n-propyl, isopropyl, ethenyl, n-propenyl, isopropenyl, ethynyl, propynyl, —OC(O)CH₃, —OC(O)CH₂CH₃, —OC(O)CH(CH₃)₂, —OC(O)CH₂CH₂CH₃, —OP(O)(OR³²)₂, —OSO₂R³³, —C(O)CH₃, —C(O))CH₂CH₃, —C(O)CH(CH₃)₂, —C(O)CH₂CH₂CH₃, —CO₂R³⁴, —C(O)NR^{35a}R^{35b}, —CH₂C(O)NR^{35a}R^{35b}, —CH₂CH₂C(O)NR^{35a}R^{35b}, —CH(CH₃)CH₂C(O)NR^{35a}R^{35b}, —CH₂C(O)NR^{35a}R^{35b}, —OC(O)NR^{35a}R^{35b}, —CH₂OC(O)NR^{35a}R^{35b}, —CH₂CH₂OC(O)NR^{35a}R^{35b}, —CH(CH₃)CH₂OC(O)NR^{35a}R^{35b}, —CH₂CH₂CH₂OC(O)NR^{35a}R^{35b}, Cy¹, Ar¹, —CH₂Ar¹, —CH₂CH₂Ar¹, —CH(CH₃)CH₂Ar¹, —CH₂CH₂Ar¹, and —OAr¹. In an even further aspect, R¹ is selected from hydrogen, —F, —Cl, —OH, —CN, —NC, —NCO, —OCN, —NO₂, —ONO₂, —ONO, —NO, —N₃, —NH₂, —NH₃, —N═NR³¹, —NHOH, methyl, ethyl, ethenyl, ethynyl, —OC(O)CH₃, —OC(O)CH₂CH₃, —OP(O)(OR³²)₂, —OSO₂R³³, —C(O)CH₃, —C(O)CH₂CH₃, —CO₂R³⁴, —C(O)NR^{35a}R^{35b}, —CH₂C(O)NR^{35a}R^{35b}, —CH₂CH₂C(O)NR^{35a}R^{35b}, —OC(O)NR^{35a}R^{35b}, —CH₂OC(O)NR^{35a}R^{35b}, —CH₂CH₂OC(O)NR^{35a}R^{35b}, Cy¹, Ar², —CH₂Ar¹, —CH₂CH₂Ar¹, and —OAr¹. In a still further aspect, R¹ is selected from hydrogen, —F, —Cl, —OH, —CN, —NC, —NCO, —OCN, —NO₂, —ONO₂, —ONO, —NO, —N₃, —NH₂, —NH₃, —N═NR, —NHOH, methyl, —OC(O)CH₃, —OP(O)(OR³²)₂, —OSO₂R³³, —C(O)CH₃, —CO₂R³⁴, —C(O)NR^{35a}R_{35b}, —CH₂C(O)NR^{35a}R^{35b}, —OC(O)NR^{35a}R^{35b}, —CH₂OC(O)NR^{35a}R^{35b}, Cy¹, Ar¹, —CH₂Ar¹, and —OAr¹.

In various aspects, R¹ is selected from hydrogen, halogen, —OH, —CN, —NC, —NCO, —OCN, —NO₂, —ONO₂, —ONO, —NO, —N₃, —NH₂, —NH₃, —N═NR_, —NHOH, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, (C1-C12 alkyl)OC(O)NR^{35a}R^{35b}, Cy¹, Ar¹, (C1-C12 alkyl)Ar¹, and —OAr¹. In a further aspect, R¹ is selected from hydrogen, halogen, —OH, —CN, —NC, —NCO, —OCN, —NO2, —ONO₂, —ONO, —NO, —N3, —NH2, —NH3, —N═NR³, —NHOH, C1-C8 hydroxy, C1-C8 alkoxy, C1-C8 thioalkyl, C1-C8 allylthiol, C1-C8 aminoalkyl, C1-C8 alkylamino, (C1-C8)(C1-C8) dialkylamino, —OC(O)(C1-C8 alkyl), —OP(O)(OR³²)₂, —OSO₂R³³, —C(O)(C1-C8 alkyl), —CO₂R³⁴, —C(O)NR^{35a}R^{35b}, —(C1-C8 alkyl)C(O)NR^{35a}R^{35b}, —OC(O)NR^{35a}R^{35b}, —(C1-C8 alkyl)OC(O)NR^{35a}R^{35b}, Cy¹, Ar¹, (C1-C8 alkyl)Ar¹, and —OAr¹. In a still further aspect, R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C4 hydroxy, C1-C4 alkoxy, C1-C4 thioalkyl, C1-C4 allylthiol, C1-C4 aminoalkyl, C1-C4 alkylamino, (C1-C4)(C1-C4) dialkylamino, —OC(O)(C1-C4 alkyl), —OP(O)(OR³²)₂, —OSO₂R³³, —C(O)(C1-C4 alkyl), —CO₂R³⁴, —C(O)NR^{35a}R^{35b}, —(C1-C8 alkyl)C(O)NR^{35a}R^{35b}, —OC(O)NR^{35a}R^{35b}, —(C1-C4 alkyl)OC(O)NR^{35a}R^{35b}, Cy¹, Ar¹, (C1-C4 alkyl)Ar¹, and —OAr¹. In yet a further aspect, R¹ is selected from hydrogen, —F, —Cl, —OH, —CN, —NC, —NCO, —OCN, —NO₂, —ONO₂, —ONO, —NO, —N₃, —NH₂, —NH₃, —N═NR³¹, —NHOH, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —SCH₃, —SCH₂CH₃, —SCH(CH₃)₂, —SCH₂CH₂CH₃, —CH₂SH, —CH₂CH₂SH, —CH(CH₃)CH₂SH, —CH₂CH₂CH₂SH, —CH₂NH₂, —CH₂CH₂NH₂, —CH(CH₃)CH₂NH₂, —CH₂CH₂CH₂NH₂, —NHCH₃, —NHCH₂CH₃, —NHCH(CH₃)₂, —NHCH₂CH₂CH₃, —N(CH₃)₂, —N(CH₃)CH₂CH₃, —N(CH₃)CH(CH₃)₂, —N(CH₃)CH₂CH₂CH₃, —OC(O)CH₃, —OC(O)CH₂CH₃, —OC(O)CH(CH₃)₂, —OC(O)CH₂CH₂CH₃, —OP(O)(OR³²)₂, —OSO₂R³³, —C(O)CH₃, —C(O)CH₂CH₃, —C(O)CH(CH₃)₂, —C(O)CH₂CH₂CH₃, —CO₂R³⁴, —C(O)NR^{35a}R^{35b}, —CH₂C(O)NR^{35a}R^{35b}, —CH₂CH₂C(O)NR^{35a}R^{35b}, —CH(CH₃)CH₂C(O)NR^{35a}R^{35b}, —CH₂CH₂CH₂C(O)NR^{35a}R^{35b}, —OC(O)NR^{35a}R^{35b}, —CH₂OC(O)NR^{35a}R^{35b}, —CH₂CH₂OC(O)NR^{35a}R^{35b}, —CH(CH₃)CH₂OC(O)NR^{35a}R^{35b}, —CH₂CH₂CH₂OC(O)NR^{35a}R^{35b}, Cy¹, Ar¹, —CH₂Ar¹, —CH₂CH₂Ar¹, —CH(CH₃)CH₂Ar¹, —CH₂CH₂CH₂Ar¹, and —OAr¹. In an even further aspect, R¹ is selected from hydrogen, —F, —Cl, —OH, —CN, —NC, —NCO, —OCN, —NO₂, —ONO₂, —ONO, —NO, —N₃, —NH₂, —NH₃, —N═NR³, —NHOH, —CH₂OH, —CH₂CH₂OH, —OCH₃, —OCH₂CH₃, —SCH₃, —SCH₂CH₃, —CH₂SH, —CH₂CH₂SH, —CH₂NH₂, —CH₂CH₂NH₂, —NHCH₃, —NHCH₂CH₃, —N(CH₃)₂, —N(CH₃)CH₂CH₃, —OC(O)CH₃, —OC(O)CH₂CH₃, —OP(O)(OR³²)₂, —OSO₂R³³, —C(O)CH₃, —C(O)CH₂CH₃, —CO₂R³⁴, —C(O)NR^{35a}R^{35b}, —CH₂C(O)NR^{35a}R^{35b}, —CH₂CH₂C(O)NR^{35a}R^{35b}, —OC(O)NR^{35a}R^{35b}, —CH₂OC(O)NR^{35a}R^{35b}, —CH₂CH₂OC(O)NR^{35a}R^{35b}, Cy¹, Ar¹, —CH₂Ar¹, —CH₂CH₂Ar¹, and —OAr¹. In a still further aspect, R¹ is selected from hydrogen, —F, —Cl, —OH, —CN, —NC, —NCO, —OCN, —NO₂, —ONO₂, —ONO, —NO, —N₃, —NH₂, —NH₃, —NHOH, —CH₂OH, —OCH₃, —SCH₃, —CH₂SH, —CH₂NH₂, —NHCH₃, —N(CH₃)₂, —OC(O)CH₃, —OP(O)(OR³²)₂, —OSO₂R³³, —C(O)CH₃, —CO₂R³⁴, C(O)NR^{35a}R^{35b}, —CH₂C(O)NR^{35a}R^{35b}, —OC(O)NR^{35a}R^{35b}, —CH₂OC(O)NR^{35a}R^{35b}, Cy¹, Ar¹, —CH₂Ar¹, and —OAr¹.

In various aspects, R¹ is selected from hydrogen, halogen, C1-C12 alkyl, C2-CH₂ alkenyl, C2-C12 alkynyl, C1-C12 hydroxy. C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹. In a further aspect, R¹ is selected from hydrogen, halogen, C1-C8 alkyl, C2-C8 alkenyl. C2-C8 alkynyl, C1-C8 hydroxy, C1-C8 alkoxy, C1-C8 thioalkyl, C1-C8 alkylthiol, C1-C8 aminoalkyl, C1-C8 alkylamino, (C1-C8)(C1-C8) dialkylamino, —OC(O)(C1-C8 alkyl), —OP(O)(OR³²)₂, —OSO₂R³³, —C(O)(C1-C8 alkyl), —CO₂R³⁴, —C(O)NR^{35a}R^{35b}, —(C1-C8 alkyl)C(O)NR^{35a}R^{35b}, —OC(O)NR^{35a}R^{35b}, —(C1-C8 alkyl)OC(O)NR^{35a}R^{35b}, Cy¹, Ar¹, (C1-C8 alkyl)Ar¹, and —OAr¹. In a still further aspect, R¹ is selected from hydrogen, halogen, C1-C4 alkyl, C2-C4 alkenyl, C2-C4 alkynyl, C1-C4 hydroxy, C1-C4 alkoxy, C1-C4 thioalkyl, C1-C4 alkylthiol, C1-C4 aminoalkyl, C1-C4 alkylamino, (C1-C4)(C1-C4) dialkylamino, —OC(O)(C1-C4 alkyl), —OP(O)(OR³²)₂, —OSO₂R³³, —C(O)(C1-C4 alkyl), —CO₂R³⁴, —C(O)NR^{35a}R^{35b}, —(C1-C8 alkyl)C(O)NR^{35a}R^{35b}, —OC(O)NR^{35a}R^{35b}, —(C1-C4 alkyl)OC(O)NR^{35a}R^{35b}, Cy¹, Ar¹, (C1-C4 alkyl)Ar¹, and —OAr¹. In yet a further aspect, R¹ is selected from hydrogen, —F, —Cl, methyl, ethyl, n-propyl, isopropyl, ethenyl, n-propenyl, isopropenyl, ethynyl, propynyl, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —SCH₃, —SCH₂CH₃, —SCH(CH₃)₂, —SCH₂CH₂CH₃, —CH₂SH—, —CH₂CH₂SH, —CH(CH₃)CH₂SH, —CH₂CH₂CH₂SH, —CH₂NH₂, —CH₂CH₂NH₂, —CH(CH₃)CH₂NH₂, —CH₂CH₂CH₂NH₂, —NHCH₃, —NiCH₂CH₃, —NHCH(CH₃)₂, —NHCH₂CH₂CH₃, —N(CH₃)₂, —N(CH₃)CH₂CH₃, —N(CH₃)CH(CH₃)₂, —N(CH₃)CH₂CH₂CH₃, —OC(O)CH₃, —OC(O)CH₂CH₃, —OC(O)CH(CH₃)₂, —OC(O)CH₂CH₂CH₃, —OP(O)(OR³²)₂, —OSO₂R³³, —C(O)CH₃, —C(O)CH₂CH₃, —C(O)CH(CH₃)₂, —C(O)CH₂CH₂CH₃, —CO₂R³⁴, —C(O)NR₃₅ₐR^{35b}, —CH₂C(O)NR^{35a}R^{35b}, —CH₂CH₂C(O)NR^{35a}R^{35b}, —CH(CH₃)CH₂C(O)NR^{35a}R^{35b}, —CH₂CH₂CH₂C(O)NR^{35a}R^{35b}, —OC(O)NR^{35a}R^{35b}, —CH₂OC(O)NR^{35a}R^{35b}, —CH₂CH₂OC(O)NR^{35a}R^{35b}, —CH(CH₃)CH₂OC(O)NR^{35a}R^{35b}, —CH₂CH₂CH₂OC(O)NR^{35a}R^{35b}, Cy¹, Ar¹, —CH₂Ar¹, —CH₂CH₂Ar¹, —CH(CH₃)CH₂Ar¹, —CH₂CH₂CH₂Ar¹, and —OAr¹. In an even further aspect, R¹ is selected from hydrogen, —F, —Cl, methyl, ethyl, ethenyl, ethynyl, —CH₂OH, —CH₂CH₂OH, —OCH₃, —OCH₂CH₃, —SCH₃, —SCH₂CH₃, —CH₂SH, —CH₂CH₂SH, —CH₂NH₂, —CH₂CH₂NH₂, —NHCH₃, —NHCH₂CH₃, —N(CH₃)₂, —N(CH₃)CH₂CH₃, —OC(O)CH₃, —OC(O)CH₂CH₃, —OP(O)(OR³²)₂, —OSO₂R³³, —C(O)CH₃, —C(O)CH₂CH₃, —CO₂R³⁴, —C(O)NR^{35a}R^{35b}, —CH₂C(O)NR^{35a}R^{35b}, —CH₂CH₂C(O)NR^{35a}R^{35b}, —OC(O)NR^{35a}R^{35b}, —CH₂OC(O)NR^{35a}R_{35b}, —CH₂H₂OC(O)NR^{35a}R^{35b}, Cy¹, Ar¹, —CH₂Ar¹, —CH₂CH₂Ar¹, and —OAr¹. In a still further aspect, R¹ is selected from hydrogen, —F, —Cl, methyl, —CH₂OH, —OCH₃, —SCH₃, —CH₂SH, —CH₂NH₂, —NHCH₃, —N(CH₃)₂, —OC(O)CH₃, —OP(O)(OR³²)₂, —OSO₂R³³, —C(O)CH₃, —CO₂R³⁴, —C(O)NR^{35a}R_{35b}, —CH₂C(O)NR^{35a}R^{35b}, —OC(O)NR^{35a}R^{35b}, —CH₂OC(O)NR^{35a}R^{35b}, Cy¹, Ar¹, —CH₂Ar¹, and —OAr¹.

In various aspects, R¹ is selected from hydrogen, —OH, C1-C12 hydroxy, C1-C12 alkoxy, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹. In a further aspect, R¹ is selected from hydrogen, —OH, C1-C8 hydroxy, C1-C8 alkoxy, —OC(O)(C1-C8 alkyl), —OP(O)(OR³²)₂, —OSO₂R³³, —C(O)(C1-C8 alkyl), —CO₂R³⁴, —C(O)NR^{35a}R^{35b}, —(C1-C8 alkyl)C(O)NR₃₅ₐR^{35b}, —OC(O)NR^{35a}R^{35b}, —(C1-C8 alkyl)OC(O)NR₃₅ₐR^{35b}, Cy¹, Ar¹, (C1-C8 alkyl)Ar¹, and —OAr¹. In a still further aspect, R¹ is selected from hydrogen, —OH, C1-C4 hydroxy, C1-C4 alkoxy, —OC(O)(C1-C4 alkyl), —OP(O)(OR³²)₂, —OSO₂R³, —C(O)(C1-C4 alkyl), —CO₂R³⁴, —C(O)NR^{35a}R^{35b}, —(C1-C8 alkyl)C(O)NR^{35a}R^{35b}, —OC(O)NR^{35a}R^{35b}, —(C1-C4 alkyl)OC(O)NR^{35a}R^{35b}, Cy¹, Ar¹, (C1-C4 alkyl)Ar¹, and —OAr¹. In yet a further aspect, R¹ is selected from hydrogen, —OH, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, —C(O)CH(CH₃)₂, —C(O)CH₂CH₂CH₃, —OP(O)(OR³²)₂, —OSO₂R³³, —C(O)CH₃, —C(O)CH₂CH₃, —C(O)CH(CH₃)₂, —C(O)CH₂CH₂CH₃, —CO₂R³⁴, —C(O)NR^{35a}R^{35b}, —CH₂CH₂CH₂C(O)NR^{35a}R^{35b}, —CH₂CH₂C(O)NR^{35a}R^{35b}, —CH(CH₃)CH₂C(O)NR^{35a}R^{35b}, —CH₂CH₂CH₂C(O)NR^{35a}R^{35b}, —OC(O)NR^{35a}R^{35b}, —CH₂OC(O)NR^{35a}R^{35b}, —CH₂CH₂OC(O)NR^{35a}R^{35b}, —CH(CH₃)CH₂OC(O)NR^{35a}R^{35b}, —CH₂CH₂CH₂OC(O)NR^{35a}R^{35b}, Cy¹, Ar¹, —CH₂Ar¹, —CH₂CH₂Ar¹, —CH(CH₃)CH₂Ar¹, —CH₂CH₂CH₂Ar¹, and —OAr¹. In an even further aspect, R¹ is selected from hydrogen, —OH, —CH₂OH, —CH₂CH₂OH, —OCH₃, —OCH₂CH₃, —OC(O)CH₃, —OC(O)CH₂CH₃, —OP(O)(OR³²)₂, —OSO₂R³³, —C(O)CH₃, —C(O)CH₂CH₃, —CO₂R³⁴, —C(O)NR^{35a}R^{35b}, —CH₂C(O)NR^{35a}R^{35b}, —CH₂CH₂C(O)NR^{35a}R^{35b}, —OC(O)NR^{35a}R^{35b}, —CH₂OC(O)NR^{35a}R^{35b}, —CH₂CH₂OC(O)NR^{35a}R^{35b}, Cy¹, Ar¹, —CH₂Ar¹, —CH₂CH₂Ar¹, and —OAr¹. In a still further aspect, R¹ is selected from hydrogen, —OH, —CH₂OH, —OCH, —OC(O)CH₃, —OP(O)(OR³²)₂, —OSO₂R³³, —C(O)CH₃, —CO₂R³⁴, —C(O)NR)NR^{35a}R^{35b}, —CH₂C(O)NR^{35a}R^{35b}, —OC(O)NR^{35a}R^{35b}, —CH₂OC(O)NR^{35a}R^{35b}, Cy¹, Ar¹, —CH₂Ar¹, and —OAr¹.

In various aspects, R¹ is selected from hydrogen, —OH, C1-C12 hydroxy, C1-C12 alkoxy, —OC(O)(C1-C12 alkyl), —OP(O)(OR³²), —OSO₂R³³, —OC(O)NR^{35a}R^{35b}, and —OAr¹. In a further aspect, R¹ is selected from hydrogen, —OH, C1-C8 hydroxy, C1-C8 alkoxy, —OC(O)(C1-C8 alkyl), —OP(O)(OR³²)₂, —OSO₂R³³, —OC(O)NR^{35a}R^{35b}, and —OAr¹. In a still further aspect, R¹ is selected from hydrogen, —OH, C1-C4 hydroxy, C1-C4 alkoxy, —OC(O)(C1-C4 alkyl), —OP(O)(OR³²)₂, —OSO₂R³³, —OC(O)NR^{35a}R^{35b}, and —OAr¹. In yet a further aspect, R¹ is selected from hydrogen, —OH, —CH₂OH, —CH₂CH₂OH, —CH(CH₃)CH₂OH, —CH₂CH₂CH₂OH, —OCH₃, —OCH₂CH₃, —OCH(CH₃), —OCH₂CH₂CH₃, —OC(O)CH₃, —OC(O)CH₂—CH₃, —OC(O)CH(CH₃)₂, —OC(O)CH₂CH₂CH₃, —OP(O)(OR³²)₂, —OSO₂R³³, —OC(O)NR^{35a}R^{35b}, and —OAr¹. In an even further aspect, R¹ is selected from hydrogen, —OH, —CH₂OH, —CH₂CH₂CH₂OH, —OCH₃, —OCH₂CH₃, —OC(O)CH₃, —OC(O)CH₂CH₃, —OP(O)(OR³²)₂, —OSO₂R³³, —OC(O)NR^{35a}R^{35b}, and —OAr¹. In a still further aspect, R¹ is selected from hydrogen, —OH, —CH₂OH, —OCH₃—OC(O)CH₃, —OP(O)(OR³²)₂, —OSO₂R³³, —OC(O)NR^{35a}R^{35b}, and —OAr¹.

In various aspects, R¹ is selected from —OH, C1-C12 hydroxy, C1-C12 alkoxy, and —OC(O)(C1-C12 alkyl). In a further aspect, R¹ is selected from —OH, C1-C₈ hydroxy, C1-C8 alkoxy, and —OC(O)(C1-C8 alkyl). In a still further aspect, R¹ is selected from —OH, C1-C4 hydroxy, C1-C4 alkoxy, and —OC(O)(C1-C4 alkyl). In yet a further aspect, R¹ is selected from —OH, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In an even further aspect, R¹ is selected from —OH, —CH₂OH, —CH₂CH₂OH, —OCH₃, —OCH₂CH₃, —OC(O)CH₃, and —OC(O)CH₂CH₃. In a still further aspect, R¹ is selected from —OH, —CH₂OH, —OCH₃, and —OC(O)CH₃.

In various aspects, R¹ is selected from —OH and —OC(O)(C1-C12 alkyl). In a further aspect, R¹ is selected from —OH and —OC(O)(C1-C8 alkyl). In a still further aspect, R¹ is selected from —OH and —OC(O)(C1-C4 alkyl). In yet a further aspect, R¹ is selected from —OH, —OC(O)CH₃, —C(O)CH₂CH₃, —OC(O)CH(CH₃)₂, and —OC(O)CH₂CH₂CH₃. In an even further aspect, R¹ is selected from —OH, —OC(O)CH₃, and —OC(O)CH₂CH₃. In a still further aspect, R¹ is selected from —OH and —OC(O)CH₃.

In various aspects, R¹ is selected from —OC(O)(C1-C12 alkyl). In a further aspect, R¹ is selected from —OC(O)(C1-C8 alkyl). In a still further aspect, R¹ is selected from —OC(O)(C1-C4 alkyl). In yet a further aspect, R¹ is selected from —OC(O)CH₃, —OC(O)CH₂CH₃, —OC(O)CH(CH₃), and —OC(O)CH₂CH₂CH₂CH₃. In an even further aspect, R¹ is selected from —OC(O)CH₃ and —OC(O)CH₂CH₃. In a still further aspect, R¹ is —OC(O)CH₃.

f. R² and R³ Groups

In one aspect, each of R² and R³ is independently selected from hydrogen, —OH. C1-C12 hydroxy, and halogen. In a further aspect, each of R² and R³ is independently selected from hydrogen, —OH, C1-C6 hydroxy, and halogen. In a still further aspect, each of R₂ and R₃ is independently selected from hydrogen, —OH, C1-C4 hydroxy, and halogen. In yet a further aspect, each of R² and R³ is hydrogen.

In one aspect, each of R₂ and R₃ together comprise —O—.

In a further aspect, each of R² and R³ is independently selected from hydrogen, —OH, and C1-C12 hydroxy. In a still further aspect, each of R² and R³ is independently selected from hydrogen, —OH, and C1-C6 hydroxy. In yet a further aspect, each of R² and R³ is independently selected from hydrogen, —OH, and C1-C4 hydroxy. In an even further aspect, each of R² and R³ is independently selected from hydrogen, —OH, methoxy, ethoxy, n-propoxy, and isopropoxy. In a still further aspect, each of R² and R³ is independently selected from hydrogen, —OH, methoxy, and ethoxy. In yet a further aspect, each of R² and R₃ is independently selected from hydrogen, —OH, and ethoxy. In an even further aspect, each of R² and R³ is independently selected from hydrogen, —OH, and methoxy.

In a further aspect, each of R² and R³ is independently selected from hydrogen, —OH, and halogen. In a still further aspect, each of R² and R³ is independently selected from hydrogen, —OH, —F, —Cl, and —Br. In yet a further aspect, each of R² and R³ is independently selected from hydrogen, —OH, —F, and —Cl. In an even further aspect, each of R² and R³ is independently selected from hydrogen, —OH, and —F.

In a further aspect, each of R² and R³ is independently selected from —OH and C1-C12 hydroxy. In a still further aspect, each of R² and R³ is independently selected from —OH and C1-C6 hydroxy. In yet a further aspect, each of R² and R³ is independently selected from —OH and C1-C4 hydroxy. In an even further aspect, each of R² and R³ is independently selected from —OH, methoxy, ethoxy, n-propoxy, and isopropoxy. In a still further aspect, each of R² and R³ is independently selected from —OH, methoxy, and ethoxy. In yet a further aspect, each of R² and R³ is independently selected from —OH and ethoxy. In an even further aspect, each of R² and R³ is independently selected from —OH and methoxy.

In a further aspect, each of R² and R³ is independently selected from hydrogen and halogen. In a still further aspect, each of R² and R³ is independently selected from hydrogen, —F, —Cl, and —Br. In yet a further aspect, each of R² and R³ is independently selected from hydrogen, —F, and —Cl. In an even further aspect, each of R² and R³ is independently selected from hydrogen and —Cl. In a still further aspect, each of R² and R³ is independently selected from hydrogen and —F.

g. R⁵ Groups

In one aspect, R⁵ is selected from hydrogen, —OH, —NH₂, C1-C9 alkyl, C1-C9 hydroxy, C1-C9 alkoxy, C1-C9 aminoalkyl, C1-C9 alkylamino, and (C1-C9)(C1-C9) dialkylamino. In a further aspect, R⁵ is selected from hydrogen, —OH, —NH₂, C1-C6 alkyl, C1-C6 hydroxy, C1-C6 alkoxy, C1-C6 aminoalkyl, C1-C6 alkylamino, and (C1-C6)(C1-C6) dialkylamino. In a still further aspect, R is selected from hydrogen, —OH, —NH₂, C1-C4 alkyl, C1-C4 hydroxy, C1-C4 alkoxy, C1-C4 aminoalkyl, C1-C4 alkylamino, and (C1-C4)(C1-C4) dialkylamino. In yet a further aspect, R is selected from hydrogen, —OH, —NIH, methyl, ethyl, n-propyl, isopropyl, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —CH₂NH₂, —CH₂CH₂NH₂, —CH(CH₃)CH₂NH₂, —CH₂CH₂CH₂NH₂, —NHCH₃, —NHCH₂CH₃, —NHCH(CH₃)₂, —NHCH₂CH₂CH₃, —N(CH₃)₂, —N(CH₃)CH₂CH₃, —N(CH₃)CH(CH₃), and —N(CH₃)CH₂CH₂CH₃. In an even further aspect, R is selected from hydrogen, —OH, —NH₂, methyl, ethyl, —CH₂OH, —CH₂CH₂OH, —OCH₃, —OCH₂CH₃, —CH₂NH₂, —CH₂CH₂NH₂, —NHCH₃, —NHCH₂CH₃, —N(CH₃), and —N(CH₃)CH₂CH₃. In a still further aspect, R is selected from hydrogen, —OH, —NH₂, methyl, —CH₂OH, —OCH₃, —CH₂NH₂, —NHCH₃, and —N(CH₃)₂.

In one aspect, R⁵ is absent.

In various aspects, R⁵ is selected from hydrogen, —OH, —NH₂, C1-C9 alkyl, C1-C9 hydroxy, and C1-C9 alkoxy. In a further aspect, R is selected from hydrogen, —OH, —NH₂, C1-C6 alkyl, C1-C6 hydroxy, and C1-C6 alkoxy. In a still further aspect, R is selected from hydrogen, —OH, —NH₂, C1-C4 alkyl, C1-C4 hydroxy, and C1-C4 alkoxy. In yet a further aspect, R⁵ is selected from hydrogen, —OH, —NH₂, methyl, ethyl, n-propyl, isopropyl, —CH₂OH, —CH₂CH₂OH, —CH(CH₃)CH₂OH, —CH₂CH₂CH₂OH, —OCH₃, —OCH₂CH₃, —OCH(CH₃)₂, and —OCH₂CH₂CH₃. In an even further aspect, R⁵ is selected from hydrogen, —OH, —NH₂, methyl, ethyl, —CH₂OH, —CH₂CH₂OH, —OCH₃, and —OCH₂CH₃. In a still further aspect, R⁵ is selected from hydrogen, —OH, —NH₂, methyl, —CH₂OH, and —OCH₃.

In various aspects, R⁵ is selected from hydrogen, —OH, —NH₂, C1-C9 aminoalkyl, C1-C9 alkylamino, and (C1-C9)(C1-C9) dialkylamino. In a further aspect, R⁵ is selected from hydrogen, —OH, —NH₂, C1-C6 aminoalkyl, C1-C6 alkylamino, and (C1-C6)(C1-C6) dialkylamino. In a still further aspect, R is selected from hydrogen, —OH, —NH₂, C1-C4 aminoalkyl, C1-C4 alkylamino, and (C1-C4)(C1-C4) dialkylamino. In yet a further aspect, R⁵ is selected from hydrogen, —OH, —NH, —CH₂NH, —CH₂CH₂NH₂, —CH(CH₃)CH₂NH₂, —CH₂CH₂CH₂NH₂, —NHCH₃, —NHCH₂CH₃, —NHCH(CH₃)₂, —NHCH₂CH₂CH₃, —N(CH₃)₂, —N(CH₃)CH₂CH₃, —N(CH₃)CH(CH₃)₂, and —N(CH₃)CH₂CH₂CH₃. In an even further aspect, R⁵ is selected from hydrogen, —OH, —NH₂, —CH₂NH₂, —CH₂CH₂NH₂, —NHCH₃, —NHCH₂CH₃, —N(CH₃)₂, and —N(CH₃)CH₂CH₃. In a still further aspect, R⁵ is selected from hydrogen, —OH, —NH₂, —CH₂NH₂, —NHCH₃, and —N(CH₃)₂.

In various aspects, R⁵ is selected from hydrogen, —OH, —NH₂, and C1-C9 alkyl. In a further aspect, R⁵ is selected from hydrogen, —OH, —NH₂, and C1-C6 alkyl. In a still further aspect, RP is selected from hydrogen, —I, —NH2, and C1-C4 alkyl. In yet a further aspect, R⁵ is selected from hydrogen, —OH, —NH₂, methyl, ethyl, n-propyl, and isopropyl. In an even further aspect, R⁵ is selected from hydrogen, —OH, —NH₂, methyl, and ethyl. In a still further aspect, R⁵ is selected from hydrogen, —OH, —NH₂, and methyl.

In various aspects, R⁵ is selected from hydrogen and C1-C9 alkyl. In a further aspect, R⁵ is selected from hydrogen and C1-C6 alkyl. In a still further aspect, R⁵ is selected from hydrogen and C1-C4 alkyl. In yet a further aspect, R⁵ is selected from hydrogen, methyl, ethyl, n-propyl, and isopropyl. In an even further aspect, R⁵ is selected from hydrogen, methyl, and ethyl. In a still further aspect, R⁵ is selected from hydrogen and ethyl. In yet a further aspect, R⁵ is selected from hydrogen and methyl.

In a further aspect, R⁵ is hydrogen.

h. R⁶ and R^{6′} Groups

In one aspect, each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C0 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr¹, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)R⁴⁰, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰. In a further aspect, each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C15 alkyl, C2-C15 alkenyl, C2-C15 alkynyl, C1-C15 hydroxy, C1-C15 alkoxy, C1-C15 thioallyl, C1-C15 alkylthiol, C1-C15 aminoalkyl, C1-C15 alkylamino, (C1-C15)(C1-C15) dialkylamino, —C(O)(C1-C15 alkyl), —OP(O)(OR³²)₂, —OSO₂R³³, —CO₂R³⁴, —C(O)NR^{35a}R^{35b}, —(C1-C15 alkyl)C(O)NR^{35a}R^{35b}, —OC(O)NR^{35a}R^{35b}, —(C1-C15 alkyl)OC(O)NR^{35a}R^{35b}, Cy¹, Ar¹, —(C1-C15 alkyl)Ar¹, —OAr¹, —OC(O)(C1-C15 alkyl), —OC(O)Ar², —OC(O)(C1-C15 alkyl)Ar², —OC(O)Ar³, —OC(O)(C1-C15 alkyl)NR⁴²C(O)Ar³, —OC(O)(C1-C15 alkyl)OC(O)Ar³, —OC(O)R⁴⁰, —NR⁴¹C(O)(C1-C15 alkyl), —NR⁴¹C(O)Ar², —NR⁴¹C(O)(C1-C15 alkyl)Ar², —NR⁴¹C(O)Ar³, —NR⁴¹C(O)(C1-C15 alkyl)OC(O)Ar³, —NR⁴¹C(O)(C1-C15 alkyl)NR⁴²C(O)Ar³, and —NR⁴¹C(O)R⁴⁰. In a still further aspect, each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C8 alkyl, C2-C8 alkenyl, C2-C8 alkynyl, C1-C8 hydroxy, C1-C8 alkoxy, C1-C8 thioallyl, C1-C8 alkylthiol, C1-C8 aminoallyl, C1-C8 alkylamino, (C1-C8)(C1-C8) dialkylamino, —C(O)(C1-C8 allyl), —OP(O)(OR³²)₂, —OSO₂R³³, —CO₂R³⁴, —C(O)NR^{35a}R^{35b}, —(C1-C8 alkyl)C(O)NR^{35a}R^{35b}, —OC(O)NR^{35a}R^{35b}, —(C1-C8 alkyl)OC(O)NR^{35a}R^{35b}, Cy¹, Ar¹, —(C1-C8 alkyl)Ar¹, —OAr¹, —OC(O)(C1-C8 alkyl), —OC(O)Ar², —OC(O)(C1-C8 alkyl)Ar², —OC(O)Ar³, —OC(O)(C1-C8 alkyl)NR⁴²C(O)Ar³, —OC(O)(C1-C8 alkyl)OC(O)Ar³, —OC(O)R⁴⁰, —NR⁴¹C(O)(C1-C8 alkyl), —NR⁴¹C(O)Ar², —NR⁴¹C(O)(C1-C8 alkyl)Ar², —NR⁴¹C(O)Ar³, —NR⁴¹C(O)(C1-C8 alkyl)OC(O)Ar³, —NR⁴¹C(O)(C1-C8 alkyl)NR⁴²C(O)Ar³, and —NR⁴¹C(O)R⁴⁰. In yet a further aspect, each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C4 allyl, C2-C4 alkenyl, C2-C4 alkynyl, C1-C4 hydroxy, C1-C4 alkoxy, C1-C4 thioalkyl, C1-C4 alkylthiol, C1-C4 aminoalkyl, C1-C4 alkylamino, (C1-C4)(C1-C4) diallylamino, —C(O)(C1-C4 alkyl), —OP(O)(OR³²)₂, —OSO₂R³³, —CO₂R³⁴, —C(O)NR^{35a}R^{35b}, —(C1-C4 alkyl)C(O)NR^{35a}R^{35b}, —OC(O)NR^{35a}R^{35b}, —(C1-C4 alkyl)OC(O)NR^{35a}R^{35b}, Cy¹, Ar¹, —(C1-C4 alkyl)Ar¹, —OAr¹, —OC(O)(C1-C4 allyl), —OC(O)Ar², —OC(O)(C1-C4 alkyl)Ar², —OC(O)Ar³, —OC(O)(C1-C4 alkyl)NR⁴²C(O)Ar³, —OC(O)(C1-C4 alkyl)OC(O)Ar³, —OC(O)R⁴⁰, —NR⁴¹C(O)(C1-C4 alkyl), —NR⁴¹C(O)Ar², —NR⁴¹C(O)(C1-C4 alkyl)Ar², —NR⁴¹C(O)Ar³, —NR⁴¹C(O)(C1-C4 alkyl)OC(O)Ar³, —NR⁴¹C(O)(C1-C4 alkyl)NR⁴²C(O)Ar³, and —NR⁴¹C(O)R⁴⁰. In an even further aspect, each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, methyl, ethyl, n-propyl, isopropyl, ethenyl, n-propenyl, isopropenyl, ethynyl, propynyl, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —SCH₃, —SCH₂CH₃, —SCH(CH₃)₂, —SCH₂CH₂CH₃, —CH₂SH, —CH₂CH₂SH, —CH(CH₃)CH₂SH, —CH₂CH₂CH₂SH, —CH₂NH₂, —CH₂CH₂NH₂, —CH(CH₃)CH₂NH₂, —CH₂CH₂CH₂NH₂, —NHCH₃, —NHCH₂CH₃, —NHCH(CH₃)₂, —NHCH₂CH₂CH₃, —N(CH₃)₂, —N(CH₃)CH₂CH₃, —N(CH₃)CH(CH₃)₂, —N(CH₃)CH₂CH₂CH₃, —C(O)CH₃, —C(O)CH₂CH₃, —C(O)CH(CH₃)₂, —C(O)CH₂CH₂CH₃, —OP(O)(OR³²)₂, —OSO₂R³³, —CO₂R³⁴, —C(O)NR^{35a}R^{35b}, —CH₂C(O)NR^{35a}R^{35b}, —CH₂CH₂C(O)NR^{35a}R^{35b}, —CH(CH₃)CH₂C(O)NR^{35a}R^{35b}, —CH₂CH₂CH₂C(O)NR^{35a}R^{35b}, —OC(O)NR^{35a}R^{35b}, —CH₂OC(O)NR^{35a}R^{35b}, —CH₂CH₂OC(O)NR^{35a}R^{35b}, —CH(CH₃)CH₂OC(O)NR^{35a}R^{35b}, —CH₂CH₂CH₂OC(O)NR^{35a}R^{35b}, Cy¹, Ar¹, —CH₂Ar¹, —CH₂CH₂Ar¹, —CH(CH₃)CH₂Ar¹, —CH₂CH₂CH₂Ar¹, —OAr¹, —OC(O)CH₃, —OC(O)CH₂CH₃, —OC(O)CH(CH₃)₂, —OC(O)CH₂CH₂CH₃, —OC(O)Ar², —OC(O)CH₂Ar², —OC(O)CH₂CH₂Ar², —OC(O)CH(CH₃)CH₂Ar², —OC(O)CH₂CH₂CH₂Ar², —OC(O)Ar³, —OC(O)CH₂NR⁴²C(O)Ar³, —OC(O)CH₂CH₂NR⁴²C(O)Ar³, —OC(O)CH(CH₃)CH₂NR⁴²C(O)Ar³, —OC(O)CH₂CH₂CH₂NR⁴²C(O)Ar³, —OC(O)CH₂OC(O)Ar³, —OC(O)CH₂CH₂OC(O)Ar³, —OC(O)CH(CH₃)CH₂OC(O)Ar³, —OC(O)CH₂CH₂CH₂OC(O)Ar³, —OC(O)R⁴⁰, —NR⁴¹C(O)CH₃, —NR⁴¹C(O)CH₂CH₃, —NR⁴¹C(O)CH(CH₃)₂, —NR⁴¹C(O)CH₂CH₂CH₃, —NR⁴¹C(O)Ar², —NR⁴¹C(O)CH₂Ar², —NR⁴¹C(O)CH₂CH₂Ar², —NR⁴¹C(O)CH(CH₃)CH₂Ar², —NR⁴¹C(O)CH₂CH₂CH₂Ar², —NR⁴¹C(O)Ar³, —NR⁴¹C(O)CH₂OC(O)Ar³, —NR⁴¹C(O)CH₂CH₂OC(O)Ar³, —NR⁴¹C(O)CH(CH₃)CH₂OC(O)Ar³, —NR⁴¹C(O)CH₂CH₂CH₂OC(O)Ar³, —NR⁴¹C(O)CH₂NR⁴²C(O)Ar³, —NR⁴¹C(O)CH₂CH₂NR⁴²C(O)Ar³, —NR⁴¹C(O)CH(CH₃)CH₂NR⁴²C(O)Ar³, —NR⁴¹C(O)CH₂CH₂CH₂NR⁴²C(O)Ar³, and —NR⁴¹C(O)R⁴⁰. In a still further aspect, each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, methyl, ethyl, ethenyl, ethynyl, —CH₂OH, —CH₂CH₂OH, —OCH₃, —OCH₂CH₃, —SCH₃, —SCH₂CH₃, —CH₂SH, —CH₂CH₂SH, —CH₂NH₂, —CH₂CH₂NH₂, —NHCH₃, —NHCH₂CH₃, —N(CH₃)₂, —N(CH₃)CH₂CH₃, —C(O)CH₃, —C(O)CH₂CH₃, —OP(O)(OR³²)₂, —OSO₂R³³, —CO₂R³⁴, —C(O)NR^{35a}R^{35b}, —CH₂C(O)NR^{35a}R^{35b}, —CH₂CH₂C(O)NR^{35a}R^{35b}, —OC(O)NR^{35a}R^{35b}, —CH₂OC(O)NR^{35a}R^{35b}, —CH₂CH₂OC(O)NR^{35a}R^{35b}, Cy¹, Ar¹, —CH₂Ar¹, —CH₂CH₂Ar¹, —OAr¹, —OC(O)CH₃, —OC(O)CH₂CH₃, —OC(O)Ar², —OC(O)CH₂Ar², —OC(O)CH₂CH₂Ar², —OC(O)Ar³, —OC(O)CH₂NR⁴²C(O)Ar², —OC(O)CH₂CH₂NR⁴²C(O)Ar³, —OC(O)CH₂OC(O)Ar³, —OC(O)CH₂CH₂OC(O)Ar³, —OC(O)R⁴⁰, —NR⁴¹C(O)CH₃, —NR⁴¹C(O)CH₂CH₃, —NR⁴¹C(O)Ar², —NR⁴¹C(O)CH₂Ar², —NR⁴¹C(O)CH₂CH₂Ar², —NR⁴¹C(O)Ar³, —NR⁴¹C(O)CH₂OC(O)Ar³, —NR⁴¹C(O)CH₂CH₂OC(O)Ar³, —NR⁴¹C(O)CH₂NR⁴²C(O)Ar³, —NR⁴¹C(O)CH₂CH₂NR⁴²C(O)Ar³, and —NR⁴¹C(O)R⁴⁰. In yet a further aspect, each of R⁶ and R^{6′} is independently selected from hydrogen, halogen, —OH, —CN, —NC, —NCO, —OCN, —NO₂, —ONO₂, —ONO, —NO, —N, —NH₂, —NH₃, —N═NR⁴¹, —NHOH, methyl, —CH₂OH, —OCH₃, —SCH₃, —CH₂SH, —CH₂NH₂, —NHCH₃, —N(CH₃)₂, —C(O)CH₃, —OP(O)(OR³²)₂, —OSO₂R³³, —CO₂R³⁴, —C(O)NR^{35a}R^{35b}, —CH₂C(O)NR^{35a}R^{35b}, —OC(O)NR^{35a}R^{35b}, —CH₂OC(O)NR^{35a}R^{35b}, Cy¹, Ar¹, —CH₂Ar¹, —OAr¹, —OC(O)CH₃, —OC(O)Ar², —OC(O)CH₂Ar², —OC(O)Ar², —OC(O)CH₂NR⁴²C(O)Ar², —OC(O)CH₂OC(O)Ar³, —OC(O)R⁴⁰, —NR⁴¹C(O)CH₃, —NR⁴¹C(O)Ar², —NR⁴¹C(O)CH₂Ar², —NR⁴¹C(O)Ar³, —NR⁴¹C(O)CH₂OC(O)Ar³, —NR⁴¹C(O)CH₂NR⁴²C(O)Ar³, and —NR⁴¹C(O)R⁴⁰.

In one aspect, one of R⁶ and R^{6′} is absent.

In a further aspect, one of R⁶ and R^{6′} is —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, or —NR⁴¹C(O)R⁴⁰. In a still further aspect, one of R⁶ and R^{6′} is —NR⁴¹C(O)(C1-C15 alkyl), —NR⁴¹C(O)Ar², —NR⁴¹C(O)(C1-C15 alkyl)Ar², —NR⁴¹C(O)Ar³, —NR⁴¹C(O)(C1-C15 alkyl)OC(O)Ar³, —NR⁴¹C(O)(C1-C15 alkyl)NR⁴²C(O)Ar³, or —NR⁴¹C(O)R⁴⁰. In yet a further aspect, one of R⁶ and R^{6′} is —NR⁴¹C(O)(C1-C8 alkyl), —NR⁴¹C(O)Ar³, —NR⁴¹C(O)(C1-C8 alkyl)Ar², —NR⁴¹C(O)Ar³, —NR⁴¹C(O)(C1-C8 alkyl)OC(O)Ar³, —NR⁴¹C(O)(C1-C8 alkyl)NR⁴²C(O)Ar³, or —NR⁴¹C(O)R⁴⁰. In an even further aspect, one of R⁶ and R^{6′} is —NR⁴¹C(O)(C1-C4 alkyl), —NR⁴¹C(O)Ar², —NR⁴¹C(O)(C1-C4 alkyl)Ar², —NR₄₁C(O)Ar³, —NR⁴¹C(O)(C1-C4 alkyl)OC(O)Ar³, —NR⁴¹C(O)(C1-C4 alkyl)NR⁴²—C(O)Ar³, or —NR⁴¹C(O)R⁴⁰. In a still further aspect, one of R⁶ and R^{6′} is —NR⁴¹C(O)CH₃, —NR⁴¹C(O)CH₂CH₃, —NR⁴¹C(O)CH(CH₃)₂, —NR⁴¹C(O)CH₂CH₂CH₃, —NR⁴¹C(O)Ar², —NR⁴¹C(O)CH₂Ar², —NR⁴¹C(O)CH₂CH₂Ar², —NR⁴¹C(O)CH(CH₃)CH₂Ar³, —NR⁴¹C(O)CH₂CH₂CH₂Ar², —NR⁴¹C(O)Ar³, —NR⁴¹C(O)CH₂OC(O)Ar³, —NR⁴¹C(O)CH₂CH₂OC(O)Ar³, —NR⁴¹C(O)CH(CH₃)CH₂OC(O)Ar³, —NR⁴¹C(O)CH₂CH₂CH₂OC(O)Ar³, —NR⁴¹C(O)CH₂NR⁴²C(O)Ar³, —NR⁴¹C(O)CH₂CH₂NR⁴²C(O)Ar³, —NR⁴¹C(O)CH(CH₃)CH₂NR⁴²C(O)Ar³, —NR⁴¹C(O)CH₂CH₂CH₂NR⁴²C(O)Ar³, or —NR⁴¹C(O)R⁴⁰. In yet a further aspect, one of R⁶ and R^{6′} is —NR⁴¹C(O)CH₃, —NR⁴¹C(O)CH₂CH₃, —NRC(O)Ar², —NR⁴¹C(O)CH₂Ar², —NR⁴¹C(O)CH₂CH₂Ar², —NR⁴¹C(O)Ar³, —NR⁴¹C(O)CH₂OC(O)Ar³, —NR⁴¹C(O)CH₂CH₂OC(O)Ar³, —NR⁴¹C(O)CH₂NR⁴²C(O)Ar³, —NR⁴¹C(O)CH₂CH₂NR⁴²C(O)Ar³, or —NR⁴¹C(O)R⁴⁰. In an even further aspect, one of R⁶ and R^{6′} is —NR⁴¹C(O)CH₃, —NR⁴¹C(O)Ar², —NR⁴¹C(O)CH₂Ar², —NR⁴¹C(O)Ar³, —NR⁴¹C(O)CH₂OC(O)Ar³, —NR⁴¹C(O)CH₂NR⁴²C(O)Ar³, or —NR⁴¹C(O)R⁴⁰.

In a further aspect, R⁶ is selected from —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, or —NR⁴¹C(O)R⁴⁰ and R^{6′} is hydrogen. In a still further aspect, R⁶ is —NR⁴¹C(O)(C1-C15 alkyl), —NR⁴¹C(O)Ar², —NR⁴¹C(O)(C1-C15 alkyl)Ar², —NR⁴¹C(O)Ar³, —NR⁴¹C(O)(C1-C15 alkyl)OC(O)Ar³, —NR⁴¹C(O)(C1-C15 alkyl)NR⁴²C(O)Ar³, or —NR⁴¹C(O)R⁴⁰ and R^{6′} is hydrogen. In yet a further aspect, R⁶ is —NR⁴¹C(O)(C1-C8 alkyl), —NR C(O)Ar³, —NR⁴¹C(O)(C1-C8 alkyl)Ar², —NR⁴¹C(O)Ar³, —NR⁴¹C(O)(C1-C8 alkyl)OC(O)Ar³, —NR⁴¹C(O)(C1-C8 alkyl)NR⁴²C(O)Ar³, or —NR⁴¹C(O)R⁴⁰ and R^{6′} is hydrogen. In an even further aspect, R⁶ is —NR⁴¹C(O)(C1-C4 alkyl), —NR⁴¹C(O)Ar², —NR⁴¹C(O)(C1-C4 alkyl)Ar², —NR⁴¹C(O)Ar³, —NR⁴¹C(O)(C1-C4 alkyl)OC(O)Ar³, —NR⁴¹C(O)(C1-C4 alkyl)NR⁴²C(O)Ar³, or —NR⁴¹C(O)R⁴⁰ and R^{6′} is hydrogen. In a still further aspect, R⁶ is —NR⁴¹C(O)CH₃, —NR⁴¹C(O)CH₂CH₃, —NR⁴¹C(O)CH(CH₃)₂, —NR⁴¹C(O)CH₂CH₂CH₃, —NR⁴¹C(O)Ar², —NR⁴¹C(O)CH₂Ar², —NR⁴¹C(O)CH₂CH₂Ar², —NR⁴¹C(O)CH(CH₃)CH₂Ar², —NR⁴¹C(O)CH₂CH₂CH₂Ar², —NR⁴¹C(O)Ar³, —NR⁴¹C(O)CH₂OC(O)Ar³, —NR⁴¹C(O)CH₂CH₂OC(O)Ar³, —NR⁴¹C(O)CH₂(CH₃)CH₂OC(O)Ar³, —NR⁴¹C(O)CH₂CH₂CH₂OC(O)Ar³, —NR⁴¹C(O)CH₂NR⁴²C(O)Ar³, —NR⁴¹C(O)CH₂CH₂NR⁴²C(O)Ar³, —NR⁴¹C(O)CH(CH₃)CH₂NR⁴²C(O)Ar³, —NR⁴¹C(O)CH₂CH₂CH₂NR⁴²C(O)Ar³, or —NR⁴¹C(O)R⁴⁰ and R^{6′} is hydrogen. In yet a further aspect, R⁶ is —NR⁴¹C(O)CH₃, —NR⁴¹C(O)CH₂CH₃, —NR⁴¹C(O)Ar², —NR⁴¹C(O)CH₂Ar², —NR⁴¹C(O)CH₂CH₂Ar², —NR⁴¹C(O)Ar³, —NR⁴¹C(O)CH₂OC(O)Ar³, —NR⁴¹C(O)CH₂CH₂OC(O)Ar³, —NR⁴¹C(O)CH₂NR⁴²C(O)Ar³, —NR⁴¹C(O)CH₂CH₂NR⁴²C(O)Ar³, or —NR⁴¹C(O)R⁴⁰ and R6′ is hydrogen. In an even further aspect, R⁶ is —NR⁴¹C(O)CH₃, —NR⁴¹C(O)Ar², —NR⁴¹C(O)CH₂Ar², —NR⁴¹C(O)Ar³, —NR⁴¹C(O)CH₂OC(O)Ar³, —NR⁴¹C(O)CH₂NR⁴²C(O)Ar³, or —NR⁴¹C(O)R⁴⁰ and R^{6′} is hydrogen.

In a further aspect, R⁶ is selected from —NR⁴¹C(O)(C1-C30 alkyl)Ar², —NR⁴¹C(O)(C1-C30 alkyl)NR⁴²C(O)Ar³, or —NR⁴¹C(O)R⁴⁰ and wherein R^{6′} is hydrogen. In a still further aspect, R⁶ is —NR⁴¹C(O)(C1-C15 alkyl)Ar², —NR⁴¹C(O)(C1-C15 alkyl)NR⁴²C(O)Ar³, or —NR⁴¹C(O)R⁴⁰ and R^{6′} is hydrogen. In yet a further aspect, R⁶ is —NR⁴¹C(O)(C1-C5 alkyl)Ar², —NR⁴¹C(O)(C1-C8 alkyl)NR⁴²C(O)Ar³, or —NR⁴¹C(O)R⁴⁰ and R^{6′} is hydrogen. In an even further aspect, R⁶ is —NR⁴¹C(O)(C1-C4 alkyl)Ar², —NR⁴¹C(O)(C1-C4 alkyl)NR⁴²C(O)Ar³, or —NR⁴¹C(O)R⁴⁰ and R^{6′} is hydrogen. In a still further aspect, R⁶ is —NR⁴¹C(O)CH₂Ar², —NR⁴¹C(O)CH₂CH₂NR⁴²C(O)Ar³, —NR⁴¹C(O)CH(CH₃)CH₂Ar², —NR⁴¹C(O)CH₂CH₂CH₂Ar², —NR⁴¹C(O)CH₂NR⁴²C(O)Ar³, —NR⁴¹C(O)CH₂CH₂NR⁴²C(O)Ar³, —NR⁴¹C(O)CH(CH₃)CH₂NR⁴²C(O)Ar³, —NR⁴¹C(O)CH₂CH₂CH₂NR⁴²C(O)Ar³, or —NR⁴¹C(O)R⁴⁰ and R^{6′} is hydrogen. In yet a further aspect, R⁶ is —NR⁴¹C(O)CH₂Ar², —NR⁴¹C(O)CH₂CH₂Ar², —NR⁴¹C(O)CH₂NR⁴²C(O)Ar³, —NR⁴¹C(O)CH₂CH₂NR⁴²C(O)Ar³, or —NR⁴¹C(O)R⁴⁰ and R^{6′} is hydrogen. In an even further aspect, R⁶ is —NR⁴¹C(O)CH₂Ar², —NR⁴¹C(O)CH₂NR⁴²C(O)Ar³, or —NR⁴¹C(O)R⁴⁰ and R^{6′} is hydrogen.

In a further aspect, R⁶ is selected from —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰. In a still further aspect, R⁶ is —NR⁴¹C(O)(C1-C15 alkyl), —NR⁴¹C(O)Ar², —NR⁴¹C(O)(C1-C15 alkyl)Ar², —NR⁴¹C(O)Ar³, —NR⁴¹C(O)(C1-C15 alkyl)OC(O)Ar³, —NR⁴¹C(O)(C1-C15 alkyl)NR⁴²C(O)Ar³, or —NR⁴¹C(O)R⁴⁰. In yet a further aspect, R⁶ is —NR⁴¹C(O)(C1-C8 alkyl), —NR⁴¹C(O)Ar², —NR⁴¹C(O)(C1-C8 alkyl)Ar², —NR⁴¹C(O)Ar³, —NR⁴¹C(O)(C1-C8 alkyl)OC(O)Ar³, —NR⁴¹C(O)(C1-C8 alkyl)NR⁴²C(O)Ar³, or —NR⁴¹C(O)R⁴⁰. In an even further aspect, R⁶ is —NR⁴¹C(O)(C1-C4 alkyl), —NR⁴¹C(O)Ar², —NR⁴¹C(O)(C1-C4 alkyl)Ar², —NR⁴¹C(O)Ar³, —NR⁴¹C(O)(C1-C4 alkyl)OC(O)Ar³, —NR⁴¹C(O)(C1-C4 alkyl)NR⁴²C(O)Ar³, or —NR⁴¹C(O)R⁴⁰. In a still further aspect, R⁶ is —NR⁴¹C(O)CH₃, —NR⁴¹C(O)CH₂CH₃, —NR⁴¹C(O)CH(CH₃)₂, —NR⁴¹C(O)CH₂CH₂CH₃, —NR⁴¹C(O)Ar², —NR⁴¹C(O)CH₂Ar², —NR⁴¹C(O)CH₂CH₂Ar², —NR⁴¹C(O)CH(CH₃)CH₂Ar², —NR⁴¹C(O)CH₂CH₂CH₂Ar², —NR⁴¹C(O)Ar³, —NR⁴¹C(O)CH₂OC(O)Ar³, —NR⁴¹C(O)CH₂CH₂OC(O)Ar³, —NR⁴¹C(CO)CH(CH₃)CH₂OC(O)Ar³, —NR⁴¹C(O)CH₂CH₂CH₂OC(O)Ar³, —NR⁴¹C(O)CH₂NR⁴²C(O)Ar³, —NR⁴¹C(O)CH₂CH₂NR⁴²C(O)Ar³, —NR⁴¹C(O)CH(CH₃)CH₂NR⁴²C(O)Ar³, —NR⁴¹C(O)CH₂CH₂CH₂NR⁴²C(O)Ar³, or —NR⁴¹C(O)R⁴⁰. In yet a further aspect, R⁶ is —NR⁴¹C(O)CH₃, —NR⁴¹C(O)CH₂CH₃, —NR⁴¹C(O)Ar², —NR⁴¹C(O)CH₂Ar², —NR⁴¹C(O)CH₂CH₂Ar², —NR⁴¹C(O)Ar³, —NR⁴¹C(O)CH₂OC(O)Ar³, —NR⁴¹C(O)CH₂CH₂OC(O)Ar³, —NR⁴¹C(O)CH₂NR⁴²C(O)Ar³, —NR⁴¹C(O)CH₂CH₂NR⁴²C(O)Ar³, or —NR⁴¹C(O)R⁴⁰. In an even further aspect, R⁶ is —NR⁴¹C(O)CH₃, —NR⁴¹C(O)Ar², —NR⁴¹C(O)CH₂Ar², —NR⁴¹C(O)Ar³, —NR⁴¹C(O)CH₂OC(O)Ar³, —NR⁴¹C(O)CH₂NR⁴²C(O)Ar³, or —NR⁴¹C(O)R⁴⁰.

In a further aspect, R⁶ is —NR⁴¹C(O)(C1-C30 alkyl)Ar². In a still further aspect, R⁶ is —NR⁴¹C(O)(C1-C15 alkyl)Ar². In yet a further aspect, R⁶ is —NR⁴¹C(O)(C1-C8 alkyl)Ar². In an even further aspect, R⁶ is —NR⁴¹C(O)(C1-C4 alkyl)Ar². In a still further aspect, R⁶ is —NR⁴¹C(O)CH₂Ar², —NR⁴¹C(O)CH₂CH₂Ar², —NR⁴¹C(O)CH(CH₃)CH₂Ar², or —NR⁴¹C(O)CH₂CH₂CH₂Ar². In yet a further aspect, R⁶ is —NR⁴¹C(O)CH₂Ar² or —NR⁴¹C(O)CH₂CH₂Ar². In an even further aspect, R⁶ is —NRC(O)CH₂Ar².

In a further aspect, R⁶ is —NR⁴¹C(O)(C1-C30 alkyl)NR⁴²C(O)Ar³. In a still further aspect, R⁶ is —NR⁴¹C(O)(C1-C15 alkyl)NR⁴¹C(O)Ar³. In yet a further aspect, R⁶ is —NR⁴¹C(O)(C1-C8 alkyl)NR⁴²C(O)Ar³. In an even further aspect, R⁶ is —NR⁴¹C(O)(C1-C4 alkyl)NR⁴²C(O)Ar³. In a still further aspect, R⁶ is —NR⁴¹C(O)CH₂CH₂NR⁴²C(O)Ar³, —NR⁴¹C(O)CH(CH₃)CH₂NR⁴²C(O)Ar³, or —NR⁴¹C(O)CH₂CH₂CH₂NR⁴¹C(O)Ar³. In yet a further aspect, R⁶ is —NR⁴¹C(O)CH₂NR⁴¹C(O)Ar³ or —NR⁴¹C(O)CH₂CH₂NR⁴²C(O)Ar³. In an even further aspect, R⁶ is —NR⁴¹C(O)CH₂NR⁴²C(O)Ar³.

In a further aspect, one of R⁶ and R^{6′} is —OC(O)R⁴⁰ or —NR⁴¹C(O)R⁴⁰. In a still further aspect, one of R⁶ and R^{6′} is —OC(O)R⁴⁰. In yet a further aspect, one of R⁶ and R^{6′} is —NR⁴¹C(O)R⁴⁰.

In a further aspect, R⁶ is —NR⁴¹C(O)Ar³. In a still further aspect, R⁶ is —NR⁴¹C(O)R⁴⁰.

In a further aspect, R^{6′} is hydrogen.

i. R⁷ and R^{7′} Groups

In one aspect, R⁷ is selected from hydrogen, —OH, C1-C30 hydroxy, C1-C30 alkoxy, —OC(O)(C1-C30 alkyl), and —OC(O)NR^{35a}R^{35b}, and R^{7′} is selected from hydrogen, —OH, C1-C30 hydroxy, C1-C30 alkoxy, and —OC(O)(C1-C30 alkyl). In a further aspect, R⁷ is selected from hydrogen, —OH, C1-C15 hydroxy, C1-C1S alkoxy, —OC(O)(C1-C15 alkyl), and —OC(O)NR^{35a}R^{35b}, and R^{7′} is selected from hydrogen, —OH, C1-C15 hydroxy, C1-C15 alkoxy, and —OC(O)(C1-C15 alkyl). In a still further aspect, R⁷ is selected from hydrogen, —OH, C1-C8 hydroxy, C1-C8 alkoxy, —OC(O)(C1-C8 alkyl), and —OC(O)NR^{35a}R^{35b}, and R^{7′} is selected from hydrogen, —OH, C1-C8 hydroxy, C1-C8 alkoxy, and —OC(O)(C1-C8 alkyl). In yet a further aspect, R⁷ is selected from hydrogen, —OH, C1-C4 hydroxy, C1-C4 alkoxy, —OC(O)(C1-C4 alkyl), and —OC(O)NR^{35a}R^{35b}, and R^{7′} is selected from hydrogen, —OH, C1-C4 hydroxy, C1-C4 alkoxy, and —OC(O)(C1-C4 alkyl). In an even further aspect, R⁷ is selected from hydrogen, —OH, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, —OC(O)CH₂CH₂CH₃, and —OC(O)NR^{35a}R^{35b}, and R^{7′} is selected from hydrogen, —OH, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In a still further aspect, R⁷ is selected from hydrogen, —OH, —CH₂OH, —CH₂CH₂OH, —OCH₃, —OCH₂CH₃, —OC(O)CH₃, —OC(O)CH₂CH₃, and —OC(O)NR^{35a}R^{35b}, and R^{7′} is selected from hydrogen, —OH, —CH₂OH, —CH₂CH₂OH, —OCH₃, —OCH₂CH₃, —OC(O)CH₃, and —OC(O)CH₂CH₃. In yet a further aspect, R⁷ is selected from hydrogen, —OH, —CH₂OH, —OCH₃, —OC(O)CH₃, and —OC(O)NR^{35a}R^{35b}, and R^{7′} is selected from hydrogen, —OH, —CH₂OH, —OCH₃, and —OC(O)CH₃.

In one aspect, each of R⁷ and R^{7′} together comprise ═O.

In one aspect, one of R⁷ and R^{7′} is absent.

In various aspects, R⁷ is selected from hydrogen, —OH, and —OC(O)(C1-C30 alkyl), and R^{7′} is selected from hydrogen, —OH, and —OC(O)(C1-C30 alkyl). In a further aspect, R⁷ is selected from hydrogen, —OH, and —OC(O)(C1-C15 alkyl), and R^{7′} is selected from hydrogen, —OH, and —OC(O)(C1-C15 alkyl). In a still further aspect, R⁷ is selected from hydrogen, —OH, and —OC(O)(C1-C8 alkyl, and R^{7′} is selected from hydrogen, —OH, and —OC(O)(C1-C8 alkyl). In yet a further aspect, R⁷ is selected from hydrogen, —OH, and —OC(O)(C1-C4 alkyl), and R^{7′} is selected from hydrogen, —OH, and —OC(O)(C1-C4 alkyl). In an even further aspect, R⁷ is selected from hydrogen, —OH, —OC(O)CH₃, —OC(O)CH₂CH₃, —OC(O)CH(CH₃)₂, and —OC(O)CH₂CH₂CH₃, and R^{7′} is selected from hydrogen, —OH, —OC(O)CH₃, —OC(O)CH₂CH₃, —OC(O)CH(CH₃)₂, and —OC(O)CH₂CH₂CH₃. In a still further aspect, R⁷ is selected from hydrogen, —OH, —OC(O)CH₃, and —OC(O)CH₂CH₃, and R^{7′} is selected from hydrogen, —OH, —OC(O)CH₃, and —OC(O)CH₂CH₃. In yet a further aspect, R⁷ is selected from hydrogen, —OH, and —OC(O)CH₃, and R^{7′} is selected from hydrogen, —OH, and —OC(O)CH₃.

In various aspects, R⁷ is selected from hydrogen, —OH, C1-C30 hydroxy, C1-C30 alkoxy, —OC(O)(C1-C30 alkyl), and —OC(O)NR^{35a}R^{35b}, and R^{7′} is hydrogen. In a further aspect, R⁷ is selected from hydrogen, —OH, C1-C15 hydroxy, C1-C15 alkoxy, —OC(O)(C1-C15 alkyl), and —OC(O)NR^{35a}R^{35b}, and R^{7′} is hydrogen. In a still further aspect, R⁷ is selected from hydrogen, —OH, C1-C8 hydroxy, C1-C8 alkoxy, —OC(O)(C1-C8 alkyl), and —OC(O)NR^{35a}R^{35b}, and R^{7′} is hydrogen. In yet a further aspect, R⁷ is selected from hydrogen, —OH, C1-C4 hydroxy, C1-C4 alkoxy, —OC(O)(C1-C4 alkyl), and —OC(O)NR^{35a}R^{35b}, and R^{7′} is hydrogen. In an even further aspect, R⁷ is selected rom hydrogen, —OH, —CH₂OH, —CH₂CH₂OH, —CH(CH₃)CH₂OH, —CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, —OC(O)CH₂CH₂CH₃, and —OC(O)NR^{35a}R^{35b}, and R^{7′} is hydrogen. In a still further aspect, R⁷ is selected from hydrogen. —OH, —CH₂OH, —CH₂CH₂OH, —OCH₃, —OCH₂CH₃, —OC(O)CH₃, —OC(O)CH₂CH₃, and —OC(O)NR^{35a}R^{35b}, and R^{7′} is hydrogen. In yet a further aspect, R⁷ is selected from hydrogen, —OH, —CH₂OH, —OCH₃, —OC(O)CH₃, and —OC(O)NR^{35a}R^{35b}, and R^{7′} is hydrogen.

In various aspects, R⁷ is selected from —OH and —OC(O)(C1-C30 alkyl), and R^{7′} is hydrogen. In a further aspect, R⁷ is selected from —OH and —OC(O)(C1-C15 alkyl), and R^{7′} is hydrogen. In a still further aspect, R⁷ is selected from —OH and —OC(O)(C1-C8 alkyl), and R^{7′} is hydrogen. In yet a further aspect, R⁷ is selected from —OH and —OC(O)(C1-C4 alkyl), and R^{7′} is hydrogen. In an even further aspect. R⁷ is selected from —OH, —OC(O)CH₃, —OC(O)CH₂CH₃, —OC(O)CH(CH₃)₂, and —OC(O)CH₂CH₂CH₃, and R^{7′} is hydrogen. In a still further aspect, R⁷ is selected from —OH, —OC(O)CH₃, and —OC(O)CH₂CH₃, and R^{7′} is hydrogen. In yet a further aspect, R⁷ is selected from —OH and —OC(O)CH₃, and R^{7′} is hydrogen.

In various aspects, R^{7′} is selected from hydrogen, —OH, C1-C30 hydroxy, C1-C30 alkoxy, and —OC(O)(C1-C30 alkyl), and R⁷ is hydrogen. In a further aspect, R^{7′} is selected from hydrogen, —OH, C1-C15 hydroxy, C1-C15 alkoxy, —OC(O)(C1-C15 alkyl), and R⁷ is hydrogen. In a still further aspect, R^{7′} is selected from hydrogen, —OH, C1-C8 hydroxy, C1-C8 alkoxy, and —OC(O)(C1-C8 alkyl), and R⁷ is hydrogen. In yet a further aspect, R^{7′} is selected from hydrogen, —OH, C1-C4 hydroxy, C1-C4 alkoxy, and —OC(O)(C1-C4 alkyl), and R⁷ is hydrogen. In an even further aspect, R^{7′} is selected from hydrogen, —OH, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃, and wherein R⁷ is hydrogen. In a still further aspect, R^{7′} is selected from hydrogen, —OH, —CH₂OH, —CH₂CH₂OH, —OCH₃, —OCH₂CH₃, —OC(O)CH₃, and —OC(O)CH₂CH₃, and R⁷ is hydrogen. In yet a further aspect, R^{7′} is selected from hydrogen, —OH, —CH₂OH, —OCH₃, and —OC(O)CH₃, and R⁷ is hydrogen.

In various aspects, R^{7′} is selected from —OH and —OC(O)(C1-C30 alkyl), and R⁷ is hydrogen. In a further aspect, R^{7′} is selected from —OH and —OC(O)(C1-C15 alkyl), and R⁷ is hydrogen. In a still further aspect, R^{7′} is selected from —OH and —OC(O)(C1-C8 alkyl), and R⁷ is hydrogen. In yet a further aspect, R^{7′} is selected from —OH and —OC(O)(C1-C4 alkyl), and R⁷ is hydrogen. In an even further aspect, R^{7′} is selected from —OH, —OC(O)CH₃, —OC(O)CH₂CH₃, —C(O)CH(CH₃)₂, and —OC(O)CH₂CH₂CH₃, and R⁷ is hydrogen. In a still further aspect, R^{7′} is selected from —OH, —OC(O)CH₃, and —OC(O)CH₂CH₃, and R⁷ is hydrogen. In yet a further aspect, R^{7′} is selected from —OH and —OC(O)CH₃, and R⁷ is hydrogen.

In a further aspect, R⁷ is —OH and R^{7′} is hydrogen. In a still further aspect, R^{7′} is —OH and R⁷ is hydrogen.

j. R⁸ and R^{8′} Groups

In one aspect, each of R⁸ and R^{8′} is independently selected from hydrogen, halogen, —OH, —CN, —NC, —NCO, —CN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr¹, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)(C1-C30 alkyl)-L-Z, —OC(O)(C1-C30 alkyl)-L-(C1-C30 alkyl)-Z, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, —NR⁴¹C(O)(C1-C30 alkyl)-L-Z, and —NR⁴¹C(O)(C1-C30 alkyl)-L-(C1-C30 alkyl)-Z. In a further aspect, each of R⁸ and R^{8′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C15 alkyl, C2-C15 alkenyl, C2-C15 alkynyl, C1-C15 hydroxy, C1-C15 alkoxy, C1-C15 thioalkyl, C1-C15 alkylthiol, C1-C15 aminoalkyl, C1-C15 alkylamino, (C1-C15)(C1-C15) dialkylamino, —C(O)(C1-C15 alkyl), —OP(O)(OR³²)₂, —OSO₂R³³, —CO₂R³⁴, —C(O)NR^{35a}R^{35b}, —(C1-C15 alkyl)C(O)NR^{35a}R^{35b}, —OC(O)NR^{35a}R^{35b}, —(C1-C15 alkyl)OC(O)NR^{35a}R^{35b}, Cy¹, Ar¹, —(C1-C15 alkyl)Ar¹, —OAr¹, —OC(O)(C1-C15 alkyl), —OC(O)Ar², —OC(O)(C1-C15 alkyl)Ar², —OC(O)Ar³, —OC(O)(C1-C15 alkyl)NR⁴²C(O)Ar³, —OC(O)(C1-C15 alkyl)OC(O)Ar³, —OC(O)(C1-C15 alkyl)-L-Z, —OC(O)(C1-C15 alkyl)-L-(C1l-C15 alkyl)-Z, —NR⁴¹C(O)(C1-C₁₅ alkyl), —NR⁴¹C(O)Ar², —NR⁴¹C(O)(C1-C15 alkyl)Ar², —NR⁴¹C(O)Ar³, —NR⁴¹C(O)(C1-C15 alkyl)OC(O)Ar³, —NR⁴¹C(O)(C1-C15 alkyl)NR⁴²C(O)Ar³, —NR⁴¹C(O)(C1-C15 alkyl)-L-Z, and —NR⁴¹C(O)(C1-C15 alkyl)-L-(C1-C15 alkyl)-Z. In a still further aspect, each of R⁸ and R^{8′} is independently selected from hydrogen, halogen, —OH, —CN, —NC, —NCO, —OCN, —NO2, —ONO₂, —ONO, —NO, —N₃, —NH₂, —NH₃, —N═NR⁴¹, —NHOH, C1-C8 alkyl, C2-C8 alkenyl, C2-C8 alkynyl, C1-C8 hydroxy, C1-C8 alkoxy, C1-C8 thioallyl, C1-C8 alkylthiol, C1-C8 aminoallyl, C1-C8 alkylamino, (C1-C8)(C1-C8) dialkylamino, —C(O)(C1-C8 allyl), —OP(O)(OR³²)₂, —OSO₂R³³, —CO₂R³⁴, —C(O)NR^{35a}R^{35b}, —(C1-C8 alkyl)C(O)NR^{35a}R^{35b}, —OC(O)NR^{35a}R^{35b}, —(C1-C8 alkyl)OC(O)NR^{35a}R^{35b}, Cy1, Ar1, —(C1-C8 alkyl)Ar1, —OAr1, —OC(O)(C1-C8 alkyl), —OC(O)Ar², —OC(O)(C1-C8 alkyl)Ar², —OC(O)Ar³, —OC(O)(C1-C8 alkyl)NR⁴²C(O)Ar³, —OC(O)(C1-C8 alkyl)OC(O)Ar³, —OC(O)(C1-C8 alkyl)-L-Z, —OC(O)(C1-C8 alkyl)-L-(C1-C8 alkyl)-Z, —NR⁴¹C(O)(C1-C8 allyl), —NR⁴¹C(O)Ar², —NR⁴¹C(O)(C1-C8 alkyl)Ar², —NR⁴¹C(O)Ar³, —NR⁴¹C(O)(C1-C8 alkyl)OC(O)Ar³, —NR⁴¹C(O)(C1-C8 alkyl)NR⁴²C(O)Ar³, —NR⁴¹C(O)(C1-C8 alkyl)-L-Z, and —NR⁴¹C(O)(C1-C8 alkyl)-L-(C1-C8 alkyl)-Z. In yet a further aspect, each of R⁸ and R^{8′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C4 alkyl, C2-C4 alkenyl, C2-C4 alkynyl, C1-C4 hydroxy, C1-C4 alkoxy, C1-C4 thioalkyl, C1-C4 alkylthiol, C1-C4 aminoalkyl, C1-C4 alkylamino, (C1-C4)(C1-C4) dialkylamino, —C(O)(C1-C4 alkyl), —OP(O)(OR³²)₂, —OSO₂R³³, —CO₂R³⁴, —C(O)NR^{35a}R^{35b}, —(C1-C4 alkyl)C(O)NR^{35a}R^{35b}, —OC(O)NR^{35a}R^{35b}, —(C1-C4 alkyl)OC(O)NR^{35a}R^{35b}, Cy¹, Ar¹, —(C1-C4 alkyl)Ar¹, —OAr¹, —OC(O)(C1-C4 alkyl), —OC(O)Ar², —OC(O)(C1-C4 alkyl)Ar², —OC(O)Ar³, —OC(O)(C1-C4 alkyl)NR⁴²C(O)Ar³, —OC(O)(C1-C4 alkyl)OC(O)Ar³, —OC(O)(C1-C4 alkyl)-L-Z, —OC(O)(C1-C4 alkyl)-L-(C1-C4 alkyl)-Z, —NR⁴¹C(O)(C1-C4 alkyl), —NR⁴¹C(O)Ar², —NR⁴¹C(O)(C1-C4 alkyl)Ar², —NR⁴¹C(O)Ar³, —NR⁴¹C(O)(C1-C4 alkyl)OC(O)Ar³, —NR⁴¹C(O)(C1-C4 alkyl)NR⁴²C(O)Ar³, —NR⁴¹C(O)(C1-C4 alkyl)-L-Z, and —NR⁴¹C(O)(C1-C4 alkyl)-L-(C1-C4 alkyl)-Z. In an even further aspect, each of R⁸ and R^{8′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, methyl, ethyl, n-propyl, isopropyl, ethenyl, n-propenyl, isopropenyl, ethynyl, propynyl, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —SCH₃, —SCH₂CH₃, —SCH(CH₃)₂, —SCH₂CH₂CH₃, —CH₂SH, —CH₂CH₂SH, —CH(CH₃)CH₂SH, —CH₂CH₂CH₂SH, —CH₂NH₂, —CH₂CH₂NH₂, —CH(CH₃)CH₂NH₂, —CH₂CH₂CH₂NH₂, —NHCH₃, —NHCH₂CH₃, —NHCH(CH₃)₂, —NHCH₂CH₂CH₃, —N(CH₃)₂, —N(CH₃)CH₂CH₃, —N(CH₃)CH(CH₃)₂, —N(CH₃)CH₂CH₂CH₃, —C(O)CH₃, —C(O)CH₂CH₃, —C(O)CH(CH₃)₂, —C(O)CH₂CH₂CH₃, —OP(O)(OR³²)₂, —OSO₂R³³, —CO₂R³⁴, —C(O)NR^{35a}R^{35b}, —CH₂C(O)NR^{35a}R^{35b}, —CH₂CH₂C(O)NR^{35a}R^{35b}, —CH(CH₃)CH₂C(O)NR^{35a}R^{35b}, —CH₂CH₂CH₂C(O)NR^{35a}R^{35b}, —OC(O)NR^{35a}R^{35b}, —CH₂OC(O)NR^{35a}R^{35b}, —CH₂CH₂OC(O)NR^{35a}R^{35b}, —CH(CH₃)CH₂OC(O)NR^{35a}R^{35b}, —CH₂CH₂CH₂OC(O)NR^{35a}R^{35b}, Cy¹, Ar¹, —CH₂Ar¹, —CH₂CH₂Ar¹, —CH(CH₃)CH₂Ar¹, —CH₂CH₂CH₂Ar¹, —OAr¹, —OC(O)CH₃, —OC(O)CH₂CH₃, —OC(O)CH(CH₃)₂, —OC(O)CH₂CH₂CH₃, —OC(O)Ar², —OC(O)CH2Ar², —OC(O)CH2CH2Ar², —OC(O)CH(CH₃)CH₂Ar², —OC(O)CH₂CH₂CH₂Ar², —OC(O)Ar³, —OC(O)CH₂NR⁴²C(O)Ar³, —OC(O)CH2CH2NR⁴²C(O)Ar³, —OC(O)CH(CH3)CH2NR⁴²C(O)Ar³, —OC(O)CH₂CH₂CH₂NR⁴²C(O)Ar³, —OC(O)CH₂OC(O)Ar³, —OC(O)CH₂CH₂OC(O)Ar³, —OC(O)CH(CH₃)CH₂OC(O)Ar³, —OC(O)CH₂CH₂CH₂OC(O)Ar³, —OC(O)CH₂-L-Z, —OC(O)CH₂CH₂-L-Z, —OC(O)CH(CH₃)CH₂-L-Z, —OC(O)CH₂CH₂CH₂-L-Z, —OC(O)CH₂-L-CH₂—Z, —OC(O)CH₂CH₂-L-CH₂CH₂—Z, —OC(O)CH₂CH₂-L-CH(CH₃)CH₂—Z, —OC(O)CH₂CH₂-L-CH₂CH₂CH₂—Z, —NR⁴¹C(O)CH₃, —NR⁴¹C(O)CH₂CH₃, —NR⁴¹C(O)CH(CH₃)₂, —NR⁴¹C(O)CH₂CH₂CH₃, —NR⁴¹C(O)Ar², —NR⁴¹C(O)CH2Ar², —NR⁴¹C(O)CH₂CH₂Ar², —NR⁴¹C(O)CH(CH₃)CH₂Ar², —NR⁴¹C(O)CH₂CH₂CH₂Ar², —NR⁴¹C(O)Ar³, —NR⁴¹C(O)CH₂OC(O)Ar³, —NR⁴¹C(O)CH₂CH₂OC(O)Ar³, —NR⁴¹C(O)CH(CH₃)CH₂OC(O)Ar³, —NR⁴¹C(O)CH₂CH₂CH₂OC(O)Ar³, —NR⁴¹C(O)CH₂NR⁴²C(O)Ar³, —NR⁴¹C(O)CH₂CH₂NR⁴²C(O)Ar³, —NR⁴¹C(O)CH(CH₃)CH₂NR⁴²C(O)Ar³, —NR⁴¹C(O)CH₂CH₂CH₂NR⁴²C(O)Ar³, —NR⁴¹C(O)CH₂-L-Z, —NR⁴¹C(O)CH₂CH₂-L-Z, —NR⁴¹C(O)CH(CH₃)CH₂-L-Z, —NR⁴¹C(O)CH₂CH₂CH₂-L-Z, —NR⁴¹C(O)CH₂-L-CH₂—Z, —NR⁴¹C(O)CH₂CH₂-L-CH₂CH₂—Z, —NR⁴¹C(O)CH₂CH₂-L-CH(CH₃)CH₂—Z, and —NR⁴¹C(O)CH₂CH₂-L-CH₂CH₂CH₂—Z. In a still further aspect, each of R⁸ and R^{8′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, methyl, ethyl, ethenyl, ethynyl, —CH₂OH, —CH₂CH₂OH, —OCH₃, —OCH₂CH₃, —SCH₃, —SCH₂CH₃, —CH₂SH, —CH₂CH₂SH, —CH₂NH₂, —CH₂CH₂NH₂, —NHCH₃, —NHCH₂CH₃, —N(CH₃)₂, —N(CH₃)CH₂CH₃, —C(O)CH₃, —C(O)CH₂CH₃, —OP(O)(OR³²)₂, —OSO₂R³³, —CO₂R³⁴, —C(O)NR^{35a}R^{35b}, —CH₂C(O)NR^{35a}R^{35b}, —CH₂CH₂C(O)NR^{35a}R^{35b}, —OC(O)NR^{35a}R^{35b}, —CH₂OC(O)NR^{35a}R^{35b}, —CH₂CH₂OC(O)NR^{35a}R^{35b}, Cy¹, Ar¹, —CH₂Ar¹, —CH₂CH₂Ar¹, —OAr¹, —OC(O)CH₃, —OC(O)CH₂CH₃, —OC(O)Ar², —OC(O)CH₂Ar², —OC(O)CH₂CH₂Ar², —OC(O)Ar³, —OC(O)CH₂NR⁴²C(O)Ar³, —OC(O)CH₂CH₂NR⁴²C(O)Ar³, —OC(O)CH₂OC(O)Ar³, —OC(O)CH₂CH₂OC(O)Ar³, —OC(O)CH₂-L-Z, —OC(O)CH₂CH₂-L-Z, —OC(O)CH₂-L-CH₂—Z, —OC(O)CH₂CH₂-L-CH₂CH₂—Z, —NR⁴¹C(O)CH₃, —NR⁴¹C(O)CH₂CH₃, —NR⁴¹C(O)Ar², —NR⁴¹C(O)CH₂Ar², —NR⁴¹C(O)CH₂CH₂Ar², —NR⁴¹C(O)Ar³, —NR⁴¹C(O)CH₂OC(O)Ar³, —NR⁴¹C(O)CH₂CH₂OC(O)Ar³, —NR⁴¹C(O)CH₂NR⁴²C(O)Ar³, —NR⁴¹C(O)CH₂CH₂NR⁴²C(O)Ar³, —NR⁴¹C(O)CH₂-L-Z, —NR⁴¹C(O)CH₂CH₂-L-Z, —NR⁴¹C(O)CH₂-L-CH₂—Z, and —NR⁴¹C(O)CH₂CH₂-L-CH₂CH₂—Z. In yet a further aspect, each of R⁸ and R^{8′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, methyl, —CH₂OH, —OCH₃, —SCH₃, —CH₂SH, —CH₂NH₂, —NHCH₃, —N(CH₃)₂, —C(O)CH₃, —OP(O)(OR³²)₂, —OSO₂R³³, —CO₂R³⁴, —C(O)NR^{35a}R^{35b}, —CH₂C(O)NR^{35a}R^{35b}, —OC(O)NR^{35a}R^{35b}, —CH₂OC(O)NR^{35a}R^{35b}, Cy¹, Ar¹, —CH₂Ar¹, —OAr¹, —OC(O)CH₃, —OC(O)Ar², —OC(O)CH₂Ar², —OC(O)Ar³, —OC(O)CH₂NR⁴²C(O)Ar³, —OC(O)CH₂OC(O)Ar³, —OC(O)CH₂-L-Z, —OC(O)CH₂-L-CH₂—Z, —NR⁴¹C(O)CH₃, —NR⁴¹C(O)Ar², —NR⁴¹C(O)CH₂Ar², —NR⁴¹C(O)Ar³, —NR⁴¹C(O)CH₂OC(O)Ar³, —NR⁴¹C(O)CH₂NR⁴²C(O)Ar³, —NR⁴¹C(O)CH₂-L-Z, and —NR⁴¹C(O)CH₂-L-CH₂—Z.

In one aspect, one of R⁸ and R^{8′} is absent.

In a further aspect, one of R⁸ and R^{8′} is —OC(O)(C1-C30 alkyl)-L-Z, —OC(O)(C1-C30 alkyl)-L-(C1-C30 alkyl)-Z, —NR⁴¹C(O)(C1-C30 alkyl)-L-Z, or —NR⁴¹C(O)(C1-C30 alkyl)-L-(C1-C30 alkyl)-Z. In a still further aspect, one of R⁸ and R^{8′} is —OC(O)(C1-C15 alkyl)-L-Z, —OC(O)(C1-C15 alkyl)-L-(C1-C15 alkyl)-Z, —NR⁴¹C(O)(C1-C15 alkyl)-L-Z, or —NR⁴¹C(O)(C1-C15 alkyl)-L-(C1-C15 alkyl)-Z. In yet a further aspect, one of R⁸ and R^{8′} is —OC(O)(C1-C8 alkyl)-L-Z, —OC(O)(C1-C8 alkyl)-L-(C1-C8 alkyl)-Z, —NR⁴¹C(O)(C1-C8 alkyl)-L-Z, or —NR⁴¹C(O)(C1-C8 alkyl)-L-(C1-C8 alkyl)-Z. In an even further aspect, one of R⁸ and R^{8′} is —OC(O)(C1-C4 alkyl)-L-Z, —OC(O)(C1-C4 alkyl)-L-(C1-C4 alkyl)-Z, —NR⁴¹C(O)(C1-C4 alkyl)-L-Z, or —NR⁴¹C(O)(C1-C4 alkyl)-L-(C1-C4 alkyl)-Z. In a still further aspect, one of R⁸ and R^{8′} is —OC(O)CH₂-L-Z, —OC(O)CH₂CH₂-L-Z, —OC(O)CH(CH3)CH-L-Z, —OC(O)CH₂CH₂-L-Z, —OC(O)CH₂-L-CH2-Z, —OC(O)CH₂CH₂-L-CH₂CH₂—Z, —OC(O)CH₂CH₂-L-CH(CH₃)CH₂—Z, —OC(O)CH₂CH₂-L-CH₂CH₂CH₂—Z, —NR⁴¹C(O)CH₂-L-Z, —NR⁴¹C(O)CH₂CH₂-L-Z, —NR⁴¹C(O)CH(CH₃)CH₂-L-Z, —NR⁴¹C(O)CH₂CH₂CH₂-L-Z, —NR⁴¹C(O)CH₂-L-CH₂—Z, —NR⁴¹C(O)CH₂CH₂-L-CH₂CH₂—Z, —NR⁴¹C(O)CH₂CH₂-L-CH(CH₃)CH₂—Z, or —NR⁴¹C(O)CH₂CH₂-L-CH₂CH₂CH₂—Z. In yet a further aspect, one of R⁸ and R^{8′} is —OC(O)CH₂-L-Z, —OC(O)CH₂CH₂-L-Z, —OC(O)CH₂-L-CH₂—Z, —OC(O)CH₂CH₂-L-CH₂CH₂—Z, —NR⁴¹C(O)CH₂-L-Z, —NR⁴¹C(O)CH₂CH₂-L-Z, —NR⁴¹C(O)CH₂-L-CH₂—Z, or —NR⁴¹C(O)CH₂CH₂-L-CH₂CH₂—Z. In a still further aspect, one of R⁸ and R^{8′} is —OC(O)CH₂-L-Z, —OC(O)CH₂-L-CH₂—Z, —NR⁴¹C(O)CH₂-L-Z, or —NR⁴¹C(O)CH₂-L-CH₂—Z.

In a further aspect, one of R⁸ and R^{8′} is —OC(O)(C1-C30 alkyl)-L-Z or —OC(O)(C1-C30 alkyl)-L-(C1-C30 alkyl)-Z. In a still further aspect, one of R⁸ and R^{8′} is —OC(O)(C1-C15 alky)-L-Z or —OC(O)(C1-C15 alkyl)-L-(C1-C15 alkyl)-Z. In yet a further aspect, one of R⁸ and R^{8′} is —OC(O)(C1-C8 alkyl)-L-Z or —OC(O)(C1-C8 alkyl)-L-(C1-C8 alkyl)-Z. In an even further aspect, one of R⁸ and R^{8′} is —OC(O)(C1-C4 alkyl)-L-Z or —OC(O)(C1-C4 alkyl)-L-(C1-C4 alkyl)-Z. In a still further aspect, one of R⁸ and R^{8′} is —OC(O)CH₂-L-Z, —OC(O)CH₂CH₂-L-Z, —OC(O)CH(CH₃)CH₂-L-Z, —OC(O)CH₂CH₂CH₂-L-Z, —OC(O)CH₂-L-CH₂—Z, —OC(O)CH₂CH₂-L-CH₂CH₂—Z, —OC(O)CH₂CH₂-L-CH(CH₃)CH₂—Z, or —OC(O)CH₂CH₂-L-CH₂CH₂CH₂—Z. In yet a further aspect, one of R⁸ and R^{8′} is —OC(O)CH₂-L-Z, —OC(O)CH₂CH₂-L-Z, —OC(O)CH₂-L-CH₂—Z, or —OC(O)CH₂CH₂-L-CH₂CH₂—Z. In a still further aspect, one of R⁸ and R^{8′} is —OC(O)CH₂-L-Z or —OC(O)CH₂-L-CH₂—Z.

In a further aspect, one of R⁸ and R^{8′} is —NR⁴¹C(O)(C1-C30 alkyl)-L-Z or —NR⁴¹C(O)(C1-C30 alkyl)-L-(C1-C30 alkyl)-Z. In a still further aspect, one of R⁸ and R^{8′} is —NR⁴¹C(O)(C1-C15 alkyl)-L-Z or —NR⁴¹C(O)(C1-C15 alkyl)-L-(C1-C15 alkyl)-Z. In yet a further aspect, one of R⁸ and R^{8′} is —NR⁴¹C(O)(C1-C8 alkyl)-L-Z or —NR⁴¹C(O)(C1-C8 alkyl)-L-(C1-C8 alkyl)-Z. In an even further aspect, one of R⁸ and R^{8′} is —NR⁴¹C(O)(C1-C4 alkyl)-L-Z or —NR⁴¹C(O)(C1-C4 alkyl)-L-(C1-C4 alkyl)-Z. In a still further aspect, one of R⁸ and R^{8′} is —NR⁴¹C(O)CH₂-L-Z, —NR⁴¹C(O)CH₂CH₂-L-Z, —NR⁴¹C(O)CH(CH₃)CH₂-L-Z, —NR⁴¹C(O)CH₂CH₂CH₂-L-Z, —NR⁴¹C(O)CH₂-L-CH₂—Z, —NR⁴¹C(O)CH₂CH₂-L-CH₂CH₂—Z, —NR⁴¹C(O)CH₂CH₂-L-CH(CH)CH₂—Z, or —NR⁴¹C(O)CH₂CH₂-L-CH₂CH₂CH₂—Z. In yet a further aspect, one of R⁸ and R^{1′} is —NR⁴¹C(O)CH₂-L-Z, —NR⁴¹C(O)CH₂CH₂-L-Z, —NR⁴¹C(O)CH₂-L-CH₂—Z, or —NR⁴¹C(O)CH₂CH₂-L-CH₂CH₂—Z. In a still further aspect, one of R⁸ and R^{8′} is —NR⁴¹C(O)CH₂-L-Z or —NR⁴¹C(O)CH₂-L-CH₂—Z.

In a further aspect, R⁸ is —OC(O)(C1-C30 alkyl)-L-Z, —OC(O)(C1-C30 alkyl)-L-(C1-C30 alkyl)-Z, —NR⁴¹C(O)(C1-C30 alkyl)-L-Z, or —NR⁴¹C(O)(C1-C30 alkyl)-L-(C1-C30 alkyl)-Z and R^{8′} is hydrogen. In a still further aspect, R⁸ is —OC(O)(C1-C15 alkyl)L-Z, —OC(O)(C1-C15 alkyl)-L-(C1-C15 alkyl)-Z, —NR⁴¹C(O)(C1-C15 alkyl)-L-Z, or —NR⁴¹C(O)(C1-C15 alkyl)-L-(C1-C15 alkyl)-Z and R^{8′} is hydrogen. In yet a further aspect, R⁸ is —OC(O)(C1-C8 alkyl)-L-Z, —OC(O)(C1-C8 alkyl)-L-(C1-C8 alkyl)-Z, —NR⁴¹C(O)(C1-C8 alkyl)-L-Z, or —NR⁴¹C(O)(C1-C8 alkyl)-L-(C1-C8 alkyl)-Z and R^{8′} is hydrogen. In an even further aspect, R⁸ is —OC(O)(C1-C4 alkyl)-L-Z, —OC(O)(C1-C4 alkyl)-L-(C1-C4 alkyl)-Z, —NR⁴¹C(O)(C1-C4 alkyl)-L-Z, or —NR⁴¹C(O)(C1-C4 alkyl)-L-(C1-C4 alkyl)-Z and R^{8′} is hydrogen. In a still further aspect, R⁸ is —OC(O)CH₂-L-Z, —OC(O)CH₂CH₂-L-Z, —OC(O)CH(CH₃)CH₂-L-Z, —OC(O)CH₂CH₂CH₂-L-Z, —OC(O)CH₂-L-CH₂—Z, —OC(O)CH₂CH₂-L-CH₂CH₂—Z, —OC(O)CH₂CH₂-L-CH(CH₃)CH₂—Z, —OC(O)CH₂CH₂-L-CH₂CH₂CH₂—Z, —NR⁴¹C(O)CH₂-L-Z, —NR⁴¹C(O)CH₂CH₂-L-Z, —NR⁴¹C(O)CH(CH₃)CH₂-L-Z, —NR⁴¹C(O)CH₂CH₂CH₂-L-Z, —NR⁴¹C(O)CH₂-L-CH₂—Z, —NR⁴¹C(O)CH₂CH₂-L-CH₂CH₂—Z, —NR⁴¹C(O)CH₂CH₂-L-CH(CH₃)CH₂—Z, or —NR⁴¹C(O)CH₂CH₂-L-CH₂CH₂CH₂—Z and R^{8′} is hydrogen. In yet a further aspect, R⁸ is —OC(O)CH₂-L-Z, —OC(O)CH₂CH₂-L-Z, —OC(O)CH₂-L-CH₂—Z, —OC(O)CH₂CH₂-L-CH₂CH₂—Z, —NR⁴¹C(O)CH₂-L-Z, NR⁴C(O)CH₂CH₂-L-Z, —NR⁴¹C(O)CH₂-L-CH₂—Z, or —NR⁴¹C(O)CH₂CH₂-L-CH₂CH₂—Z and R^{8′} is hydrogen. In a still further aspect, R⁸ is —OC(O)CH₂-L-Z, —OC(O)CH₂-L-CH₂—Z, —NR⁴¹C(O)CH₂-L-Z, or —NR⁴¹C(O)CH₂-L-CH₂—Z and R^{8′} is hydrogen.

In a further aspect, R^{8′} is hydrogen.

k. R¹¹ and R¹² Groups

In one aspect, each of R¹¹ and R¹² is independently selected from hydrogen, —OH, C1-C8 hydroxy, C1-C6 alkyl, C1-C8 alkoxy, and —OC(O)(C1-C8 alkyl). In a further aspect, each of R¹¹ and R¹² is independently selected from hydrogen, —OH, C1-C4 hydroxy, C1-C4 alkyl, C1-C4 alkoxy, and —OC(O)(C1-C4 alkyl). In a still further aspect, each of R¹¹ and R¹² is independently selected from hydrogen, —OH, methyl, ethyl, n-propyl, isopropyl, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH—, —OC(O)CH₂CH₃, —OC(O)CH(CH₃), and —OC(O)CH₂CH₂CH₃. In yet a further aspect, each of R¹¹ and R¹² is independently selected from hydrogen, —OH, methyl, ethyl, —CH₂OH, —CH₂OH, —OCH₃, —OCH₂CH₃, —OC(O)CH₃, and —OC(O)CH₂CH₃. In an even further aspect, each of R¹¹ and R¹² is independently selected from hydrogen, —OH, methyl, —CH₂OH, —OCH₃, and —OC(O)CH₃.

In a further aspect, each of R¹¹ and R¹² is the same. In a still further aspect, each of R¹¹ and R¹² is different.

In various aspects, each of R¹¹ and R¹² is independently selected from hydrogen, —OH, and —OC(O)(C1-C8 alkyl). In a further aspect, each of R¹¹ and R¹² is independently selected from hydrogen, —OH, and —OC(O)(C1-C4 alkyl). In a still further aspect, each of R¹¹ and R¹² is independently selected from hydrogen, —OH, —OC(O)CH₃, —OC(O)CH₂CH₃, —OC(O)CH(CH₃)₂, and —OC(O)CH₂CH₂CH₃. In yet a further aspect, each of R¹¹ and R¹² is independently selected from hydrogen, —OH, —OC(O)CH₃, and —OC(O)CH₂CH₃. In an even further aspect, each of R¹¹ and R¹² is independently selected from hydrogen, —OH, and —OC(O)CH₃.

In various aspects, each of R¹¹ and R² is independently selected from hydrogen and —OC(O)(C1-C8 alkyl). In a further aspect, each of R¹¹ and R¹² is independently selected from hydrogen and —OC(O)(C1-C4 alkyl). In a still further aspect, each of R¹¹ and R¹² is independently selected from hydrogen, —OC(O)CH₃, —OC(O)CH₂CH₃, —OC(O)CH(CH₃)₂, and —OC(O)CH₂CH₂CH₃. In yet a further aspect, each of R¹¹ and R¹² is independently selected from hydrogen, —OC(O)CH₃, and —OC(O)CH₂CH₃. In an even further aspect, each of R¹¹ and R¹² is independently selected from hydrogen and —OC(O)CH₃.

In various aspects, each of R¹¹ and R¹² is independently selected from —OH and —OC(O)(C1-C8 alkyl). In a further aspect, each of R¹¹ and R¹² is independently selected from —OH and —OC(O)(C1-C4 alkyl). In a still further aspect, each of R¹¹ and R¹² is independently selected from —OH, —OC(O)CH₃, —OC(O)CH₂CH₃, —OC(O)CH(CH₃)₂, and —OC(O)CH₂CH₂CH₃. In yet a further aspect, each of R¹¹ and R¹² is independently selected from —OH, —OC(O)CH₃, and —OC(O)CH₂CH₃. In an even further aspect, each of R¹¹ and R¹² is independently selected from —OH and —OC(O)CH₃.

In various aspects, each of R¹¹ and R¹² is independently selected from —OC(O)(C1-C8 alkyl). In a further aspect, each of R¹¹ and R¹² is independently selected from —OC(O)(C1-C4 alkyl). In a still further aspect, each of R¹¹ and R¹² is independently selected from —OC(O)CH₃, —OC(O)CH₂CH₃, —OC(O)CH(CH₃)₂, and —OC(O)CH₂CH₂CH₃. In yet a further aspect, each of R¹¹ and R¹² is independently selected from —OC(O)CH₃ and —OC(O)CH₂CH₃. In an even further aspect, each of R¹¹ and R¹² is —OC(O)CH₃.

l. R¹⁵ Groups

In one aspect, R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide). In a further aspect, R¹⁵ is selected from hydrogen, —OH, C1-C15 hydroxy, C1-C15 alkyl, C1-C15 alkoxy, —OC(O)(C1-C15 alkyl), —OC(O)NR^{35a}R^{35b}, —OC(O)Ar², —OC(O)(C1-C4 alkyl)Ar², and —OC(O)(C1-C8 azide). In a still further aspect, R¹⁵ is selected from hydrogen, —OH, C1-C8 hydroxy, C1-C8 alkyl, C1-C8 alkoxy, —OC(O)(C1-C8 alkyl), —OC(O)NR^{35a}R^{35b}, —OC(O)Ar², —OC(O)(C1-C4 alkyl)Ar², and —OC(O)(C1-C8 azide). In yet a further aspect, R¹⁵ is selected from hydrogen, —OH, C1-C4 hydroxy, C1-C4 alkyl, C1-C4 alkoxy, —OC(O)(C1-C4 alkyl), —OC(O)NR^{35a}R^{35b}, —OC(O)Ar², —OC(O)(C1-C4 alkyl)Ar², and —OC(O)(C1-C4 azide). In an even further aspect, R¹⁵ is selected from hydrogen, —OH, methyl, ethyl, n-propyl, isopropyl, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, —OC(O)CH₂CH₂CH₃, —OC(O)NR^{35a}R^{35b}, —OC(O)Ar², —OC(O)CH₂Ar², —OC(O)CH₂CH₂Ar², —OC(O)CH(CH₃)CH₂Ar², —OC(O)CH₂CH₂CH₂Ar², —CH₂N₃, —CH₂CH₂N₃, —CH(CH₃)CH₂N₃, and —CH₂CH₂CH₂N₃. In a still further aspect, R¹⁵ is selected from hydrogen, —OH, methyl, ethyl, —CH₂OH, —CH₂CH₂OH, —OCH₃, —OCH₂CH₃, —OC(O)CH₃, —OC(O)CH₂CH₃, —OC(O)NR^{35a}R^{35b}, —OC(O)Ar², —OC(O)CH₂Ar², —OC(O)CH₂CH₂Ar², —CH₂N₃, and —CH₂CH₂N₃. In yet a further aspect, R¹⁵ is selected from hydrogen, —OH, methyl, —CH₂OH, —OCH₃, —OC(O)CH₃, —OC(O)NR^{35a}R^{35b}, —OC(O)Ar², —OC(O)CH₂Ar², and —CH₂N₃.

In various aspects, R¹⁵ is selected from hydrogen, —OH, —OC(O)(C1-C30 alkyl), —OC(O)NR^{35a}R^{35b}, —OC(O)Ar², —OC(O)(C1-C4 alkyl)Ar², and —OC(O)(C1-C8 azide). In a further aspect, R¹⁵ is selected from hydrogen, —OH, —OC(O)(C1-C15 alkyl), —OC(O)NR^{35a}R^{35b}, —OC(O)Ar², —OC(O)(C1-C4 alkyl)Ar², and —OC(O)(C1-C8 azide). In a still further aspect, R¹⁵ is selected from hydrogen, —OH, —OC(O)(C1-C8 alkyl), —OC(O)NR^{35a}R^{35b}, —OC(O)Ar², —OC(O)(C1-C4 alkyl)Ar², and —OC(O)(C1-C8 azide). In yet a further aspect, R¹⁵ is selected from hydrogen, —OH, —OC(O)(C1-C4 alkyl), —OC(O)NR^{35a}R^{35b}, —OC(O)Ar², —OC(O)(C1-C4 alkyl)Ar², and —OC(O)(C1-C4 azide). In an even further aspect, R¹⁵ is selected from hydrogen, —OH, —OC(O)CH₃, —OC(O)CH₂CH₃, —OC(O)CH(CH3)₂, —OC(O)CH₂CH₂CH₃, —OC(O)NR^{35a}R^{35b}, —OC(O)Ar², —OC(O)CH₂Ar², —OC(O)CH₂CH₂Ar², —OC(O)CH(CH₃)CH2Ar², —OC(O)CH₂CH₂CH₂Ar², —CH₂N₃, —CH₂CH₂N₃, —CH(CH₃)CH₂N₃, and —CH₂CH₂CH₂N₃. In a still further aspect, R¹⁵ is selected from hydrogen, —OH, —OC(O)CH₃, —OC(O)CH₂CH₃, —OC(O)NR^{35a}R^{35b}, —OC(O)Ar², —OC(O)CH₂Ar², —OC(O)CH₂CH₂Ar², —CH₂N₃, and —CH₂CH₂N₃. In yet a further aspect, R¹⁵ is selected from hydrogen, —OH, —OC(O)CH₃, —OC(O)NR^{35a}R^{35b}, —OC(O)Ar², —OC(O)CH₂Ar², and —CH₂N₃.

In various aspects, R¹⁵ is selected from hydrogen, —OH, and —OC(O)(C1-C30 alkyl). In a further aspect, R¹⁵ is selected from hydrogen, —OH, and —OC(O)(C1-C15 alkyl). In a still further aspect, R¹⁵ is selected from hydrogen, —OH, and —OC(O)(C1-C8 alkyl). In yet a further aspect, R¹⁵ is selected from hydrogen, —OH, and —OC(O)(C1-C4 alkyl). In an even further aspect, R¹⁵ is selected from hydrogen, —OH, —OC(O)CH₃, —OC(O)CH₂CH₃, —OC(O)CH(CH₃)₂, and —OC(O)CH₂CH₂CH₃. In a still further aspect, R¹⁵ is selected from hydrogen, —OH, —OC(O)CH₃, and —OC(O)CH₂CH₃. In yet a further aspect, R¹⁵ is selected from hydrogen, —OH, and —OC(O)CH₃.

In various aspects, R¹⁵ is selected from —OH and —OC(O)(C1-C30 alkyl). In a further aspect, R¹⁵ is selected from —OH and —OC(O)(C1-C15 alkyl). In a still further aspect, R¹⁵ is selected from —OH and —OC(O)(C1-C8 alkyl). In yet a further aspect, R¹⁵ is selected from —OH and —OC(O)(C1-C4 alkyl). In an even further aspect, R¹⁵ is selected from —OH, —OC(O)CH₃, —OC(O)CH₂CH₃, —OC(O)CH(CH3)₂, and —OC(O)CH₂CH₂CH₃. In a still further aspect, R¹⁵ is selected from —OH, —OC(O)CH₃, and —OC(O)CH₂CH₃. In yet a further aspect, R¹⁵ is selected from —OH and —OC(O)CH₃.

In various aspects, R¹⁵ is selected from —OC(O)(C1-C30 alkyl). In a further aspect, R¹⁵ is selected from —OC(O)(C1-C15 alkyl). In a still further aspect, R¹⁵ is selected from —OC(O)(C1-C8 alkyl). In yet a further aspect, R¹⁵ is selected from —OC(O)(C1-C4 alkyl). In an even further aspect, R¹⁵ is selected from —OC(O)CH₃, —OC(O)CH₂CH₃, —OC(O)CH(CH3)₂, and —OC(O)CH₂CH₂CH₃. In a still further aspect, R¹⁵ is selected from —OC(O)CH₃ and —OC(O)CH₂CH₃. In yet a further aspect, R¹⁵ is —OC(O)CH₃.

In a further aspect, R¹⁵ is —OH.

m. R²⁰ Groups

In one aspect, R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl). In a further aspect, R²⁰ is selected from hydrogen, —OH, —OOH, C1-C4 alkyl, C1-C4 hydroxy, C1-C4 alkoxy, C1-C4 hydroperoxy, and —OC(O)(C1-C4 alkyl). In a still further aspect, R²⁰ is selected from hydrogen, —OH, —OOH, methyl, ethyl, n-propyl, isopropyl, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —CH₂OOH, —CH₂CH₂OOH, —CH(CH₃)CH₂OOH, —CH₂CH₂CH₂OOH, —OC(O)CH₃, —OC(O)CH₂CH₃, —OC(O)CH(CH₃)₂, and —OC(O)CH₂CH₂CH₃. In yet a further aspect, R²⁰ is selected from hydrogen, —OH, —OOH, methyl, ethyl, —CH₂OH, —CH₂CH₂OH, —OCH₃, —OCH₂CH₃, —CH₂OOH, —CH₂CH₂OOH, —OC(O)CH₃, and —OC(O)CH₂CH₃. In an even further aspect, R²⁰ is selected from hydrogen, —OH, —OOH, methyl, —CH₂OH, —OCH₃, —CH₂OOH, and —OC(O)CH₃.

In various aspects, R²⁰ is selected from hydrogen, —OH, C1-C8 alkyl, C1-C8 hydroxy, C1-C8 alkoxy, and —OC(O)(C1-C8 alkyl). In a further aspect, R²⁰ is selected from hydrogen, —OH, C1-C4 alkyl, C1-C4 hydroxy, C1-C4 alkoxy, and —OC(O)(C1-C4 alkyl). In a still further aspect, R²⁰ is selected from hydrogen, —OH, methyl, ethyl, n-propyl, isopropyl, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and OC(O)CH₂CH₂CH₃. In yet a further aspect, R²⁰ is selected from hydrogen, —OH, methyl, ethyl, —CH₂OH, —CH₂CH₂OH, —OCH₃, —OCH₂CH₃, —OC(O)CH₃, and —OC(O)CH₂CH₃. In an even further aspect, R²⁰ is selected from hydrogen, —OH, methyl, —CH₂OH, —OCH₃, and —OC(O)CH₃.

In various aspects, R²⁰ is selected from hydrogen, —OH, C1-C8 alkyl, and —OC(O)(C1-C8 alkyl). In a further aspect, R²⁰ is selected from hydrogen, —OH, C1-C4 alkyl, and —OC(O)(C1-C4 alkyl). In a still further aspect, R²⁰ is selected from hydrogen, —OH, methyl, ethyl, n-propyl, isopropyl, —OC(O)CH₂CH₃, —OC(O)CH(CH₃)₂, and —OC(O)CH₂CH₂CH₃. In yet a further aspect, R²⁰ is selected from hydrogen, —OH, methyl, ethyl, —OC(O)CH₃, and —OC(O)CH₂CH₃. In an even further aspect, R²⁰ is selected from hydrogen, —OH, methyl, and —OC(O)CH₃.

In various aspects, R²⁰ is selected from hydrogen and C1-C8 alkyl. In a further aspect, R²⁰ is selected from hydrogen and C1-C4 alkyl. In a still further aspect, R²⁰ is selected from hydrogen, methyl, ethyl, n-propyl, and isopropyl. In yet a further aspect, R²⁰ is selected from hydrogen, methyl, and ethyl. In an even further aspect, R²⁰ is selected from hydrogen and ethyl. In a still further aspect, R²⁰ is selected from hydrogen and methyl.

In various aspects, R²⁰ is selected from C1-C8 alkyl. In a further aspect, R²⁰ is selected from C1-C4 alkyl. In a still further aspect, R²⁰ is selected from methyl, ethyl, n-propyl, and isopropyl. In yet a further aspect, R²⁰ is selected from methyl and ethyl. In an even further aspect, R²⁰ is ethyl. In a still further aspect, R²⁰ is methyl.

n. R²¹ Groups

In one aspect, R²¹ is selected from hydrogen and C1-C6 alkyl. In a further aspect, R²¹ is selected from hydrogen and C1-C4 alkyl. In a still further aspect, R²¹ is selected from hydrogen, methyl, ethyl, n-propyl, and isopropyl. In yet a further aspect, R²¹ is selected from hydrogen, methyl, and ethyl. In an even further aspect, R²¹ is selected from hydrogen and ethyl. In a still further aspect, R²¹ is selected from hydrogen and methyl.

In a further aspect, R²¹ is hydrogen.

In a further aspect, R²¹ is C1-C6 alkyl. In a still further aspect, R²¹ is C1-C4 alkyl. In yet a further aspect, R21 is selected from methyl, ethyl, n-propyl, and isopropyl. In an even further aspect, R²¹ is selected from methyl and ethyl. In a still further aspect, R²¹ is ethyl. In yet a further aspect, R²¹ is methyl.

o. R²⁵ Groups

In one aspect, R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide). In a further aspect, R²⁵ is selected from hydrogen, —OH, C1-C15 hydroxy, C1-C15 alkoxy, —OC(O)(C1-C15 alkyl), —OC(O)NR^{35a}R^{35b}, —OC(O)Ar⁵, and —OC(O)(C1-C8 azide). In a still further aspect, R²⁵ is selected from hydrogen, —OH, C1-C8 hydroxy, C1-C8 alkoxy, —OC(O)(C1-C8 alkyl), —OC(O)NR^{35a}R^{35b}, —OC(O)Ar⁵, and —OC(O)(C1-C8 azide). In yet a further aspect, R²⁵ is selected from hydrogen, —OH, C1-C4 hydroxy, C1-C4 alkoxy, —OC(O)(C1-C4 alkyl), —OC(O)NR^{35a}R^{35b}, —OC(O)Ar⁵, and —OC(O)(C1-C4 azide). In an even further aspect, R²⁵ is selected from hydrogen, —OH, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, —OC(O)CH₂CH₂CH₃, —OC(O)NR^{35a}R^{35b}, —OC(O)Ar⁵, —CH₂N₃, —CH₂CH₂N₃, —CH(CH₃)CH₂N₃, and —CH₂CH₂CH₂N₃. In a still further aspect, R²⁵ is selected from hydrogen, —OH, —CH₂OH, —CH₂CH₂OH, —OCH₃, —OCH₂CH₃, —OC(O)CH₃, —OC(O)CH₂CH₃, —OC(O)NR^{35a}R^{35b}, —OC(O)Ar⁵, —CH₂N₃, and —CH₂CH₂N₃. In yet a further aspect, R²⁵ is selected from hydrogen, —OH, —CH₂OH, —OCH₃, —OC(O)CH₃, —OC(O)NR^{35a}R^{35b}, —OC(O)Ar⁵, and —CH₂N₃.

In various aspects, R²⁵ is selected from hydrogen, —OH, —OC(O)(C1-C30 alkyl), —OC(O)NR^{35a}R^{35b}, —OC(O)Ar5, and —OC(O)(C1-C8 azide). In a further aspect, R²⁵ is selected from hydrogen, —OH, —OC(O)(C1-C15 alkyl), —OC(O)NR^{35a}R^{35b}, —OC(O)Ar⁵, and —OC(O)(C1-C8 azide). In a still further aspect, R²⁵ is selected from hydrogen, —OH, —OC(O)(C1-C8 alkyl), —OC(O)NR^{35a}R³⁵6, —OC(O)Ar⁵, and —OC(O)(C1-C8 azide). In yet a further aspect, R²⁵ is selected from hydrogen, —OH, —OC(O)(C1-C4 alkyl), —OC(O)NR^{35a}R^{35b}, —OC(O)Ar2, —OC(O)(C1-C4 alkyl)Ar², and —OC(O)(C1-C4 azide). In an even further aspect, R²⁵ is selected from hydrogen, —OH, —OC(O)CH₃, —OC(O)CH₂CH₃, —OC(O)CH(CH₃)₂, —OC(O)CH₂CH₂CH₃, —OC(O)NR^{35a}R^{35b}, —OC(O)Ar⁵, —CH₂N₃, —CH₂CH₂N₃, —CH(CH₃)CH₂N₃, and —CH₂CH₂CH₂N₃. In a still further aspect, R²⁵ is selected from hydrogen, —OH, —OC(O)CH₃, —OC(O)CH₂CH₃, —OC(O)NR^{35a}R^{35b}, —OC(O)Ar⁵, —CH₂N₃, and —CH₂CH₂N₃. In yet a further aspect, R²⁵ is selected from hydrogen, —OH, —OC(O)CH₃, —OC(O)NR^{35a}R^{35b}, —OC(O)Ar⁵, and —CH₂N₃.

In various aspects, R²⁵ is selected from hydrogen, —OH, and —OC(O)(C1-C30 alkyl). In a further aspect, R²⁵ is selected from hydrogen, —OH, and —OC(O)(C1-C15 alkyl). In a still further aspect, R²⁵ is selected from hydrogen, —OH, and —OC(O)(C1-C8 alkyl). In yet a further aspect, R²⁵ is selected from hydrogen, —OH, and —OC(O)(C1-C4 alkyl). In an even further aspect, R²⁵ is selected from hydrogen, —OH, —OC(O)CH₃, —OC(O)CH₂CH₃, —OC(O)CH(CH₃)₂, and —OC(O)CH₂CH₂CH₃. In a still further aspect, R²⁵ is selected from hydrogen, —OH, —OC(O)CH₃, and —OC(O)CH₂CH₃. In yet a further aspect, R²⁵ is selected from hydrogen, —OH, and —OC(O)CH₃.

In various aspects, R²⁵ is selected from —OH and —OC(O)(C1-C30 alkyl). In a further aspect, R²⁵ is selected from —OH and —OC(O)(C1-C15 alkyl). In a still further aspect, R²⁵ is selected from —OH and —OC(O)(C1-C8 alkyl). In yet a further aspect, R²⁵ is selected from —OH and —OC(O)(C1-C4 alkyl). In an even further aspect, R²⁵ is selected from —OH, —OC(O)CH₃, —OC(O)CH₂CH₃, —OC(O)CH(CH₃)₂, and —OC(O)CH₂CH₂CH₃. In a still further aspect, R²⁵ is selected from —OH, —OC(O)CH₃, and —OC(O)CH₂CH₃. In yet a further aspect, R²⁵ is selected from —OH and —OC(O)CH₃.

In various aspects, R²⁵ is selected from —OC(O)(C1-C30 alkyl). In a further aspect, R²⁵ is selected from —OC(O)(C1-C15 alkyl). In a still further aspect, R²⁵ is selected from —OC(O)(C1-C8 alkyl). In yet a further aspect, R²⁵ is selected from —OC(O)(C1-C4 alkyl). In an even further aspect, R²⁵ is selected from —OC(O)CH₃, —OC(O)CH₂CH₃, —OC(O)CH(CH₃)₂, and —OC(O)CH₂CH₂CH₃. In a still further aspect, R²⁵ is selected from OC(O)CH₃ and —OC(O)CH₂CH₃. In yet a further aspect, R²⁵ is —OC(O)CH₃.

In a further aspect, R²⁵ is —OH.

p. R²⁶ and R^{26′} Groups

In one aspect, each of R²⁶ and R^{26′} is independently selected from hydrogen, —OH, C1-C8 hydroxy, and C1-C8 alkoxy. In a further aspect, each of R²⁶ and R^{26′} is independently selected from hydrogen, —OH, C1-C4 hydroxy, and C1-C4 alkoxy. In a still further aspect, each of R²⁶ and R^{26′} is independently selected from hydrogen, —OH, —CH₂OH, —CH₂CH₂OH, —CH(CH₃)CH₂OH, —CH₂CH₂CH₂OH, —OCH₃, —OCH₂CH₃, —OCH(CH₃)₂, and —OCH₂CH₂CH₃. In yet a further aspect, each of R²⁶ and R^{26′} is independently selected from hydrogen, —OH, —CH₂OH, —CH₂CH₂OH, —OCH₃, and —OCH₂CH₃. In an even further aspect, each of R²⁶ and R^{26′} is independently selected from hydrogen, —OH, —CH₂OH, and —OCH₃.

In various aspects, each of R²⁶ and R^{26′} is independently selected from hydrogen, —OH, and C1-C8 hydroxy. In a further aspect, each of R²⁶ and R^{26′} is independently selected from hydrogen, —OH, and C1-C4 hydroxy. In a still further aspect, each of R²⁶ and R26′ is independently selected from hydrogen, —OH, —CH₂OH, —CH₂CH₂OH, —CH(CH₃)CH₂OH, and —CH₂CH₂CH₂OH. In yet a further aspect, each of R²⁶ and R^{26′} is independently selected from hydrogen, —OH, —CH₂OH, and —CH₂CH₂OH. In an even further aspect, each of R²⁶ and R^{26′} is independently selected from hydrogen, —OH, and —CH₂OH.

In a further aspect, each of R²⁶ and R^{26′} is independently selected from hydrogen and —OH.

In one aspect, each of R²⁶ and R^{26′} together comprise ═O.

q. R²⁷ Groups

In one aspect, R²⁷ is selected from hydrogen and C1-C6 alkyl. In a still further aspect, each occurrence of R²⁷ is selected from hydrogen and C1-C4 alkyl. In yet a further aspect, R²⁷ is selected from hydrogen, methyl, ethyl, n-propyl, and isopropyl. In an even further aspect, R²⁷ is selected from hydrogen, methyl, and ethyl. In a still further aspect, R²⁷ is selected from hydrogen and ethyl. In yet a further aspect, R²⁷ is selected from hydrogen and methyl.

In a further aspect, R²⁷ is selected from C1-C6 alkyl. In yet a further aspect, R²⁷ is selected from C1-C4 alkyl. In an even further aspect, R²⁷ is selected from methyl, ethyl, n-propyl, and isopropyl. In a still further aspect, R²⁷ is selected from methyl and ethyl. In yet a further aspect, R²⁷ is ethyl. In an even further aspect, R²⁷ is methyl.

In a further aspect, R²⁷ is hydrogen.

r. R²⁸ and R²⁹ Groups

In one aspect, each of R²⁸ and R²⁹ is independently selected from hydrogen and halogen. In a further aspect, each of R²⁸ and R²⁹ is independently selected from hydrogen, —F, —Cl, and —Br. In a still further, each of R²⁸ and R²⁹ is independently selected from hydrogen, —F, and —Cl. In yet a further aspect, each of R²⁸ and R²⁹ is independently selected from hydrogen and —F. In an even further aspect, each of R²⁸ and R²⁹ is independently selected from hydrogen and —Cl.

In one aspect, each of R²⁸ and R²⁹ together comprise —O— or —N(R³⁷)—. In a further aspect, each of R²⁸ and R²⁹ together comprise —O—. In a still further aspect, each of R²⁸ and R²⁹ together comprise —N(R³⁷)—.

In a further aspect, each of R²⁸ and R²⁹ is hydrogen.

In a further aspect, each of R²⁸ and R²⁹ is independently halogen. In a still further aspect, each of R²⁸ and R²⁹ is independently selected from —F, —Cl, and —Br. In yet a further aspect, each of R²⁸ and R²⁹ is independently selected from —F and —Cl. In an even further aspect, each of R²⁸ and R²⁹ is —F. In a still further aspect, each of R²⁸ and R²⁹ is —Cl.

s. R³¹, R³², R³⁴, R^{35a}, and R^{35b} Groups

In one aspect, each occurrence of R³, R², R³⁴, R^{35a}, and R^{35b}, when present, is independently selected from hydrogen and C1-C12 alkyl. In a further aspect, each occurrence of R³¹, R³², R³⁴, R^{35a}, and R^{35b}, when present, is independently selected from hydrogen and C1-C6 alkyl. In a still further aspect, each occurrence of R³¹, R³², R³⁴, R^{3a}, and R^{35b}, when present, is independently selected from hydrogen and C1-C4 alkyl. In yet a further aspect, each occurrence of R³¹, R³², R³⁴, R^{35a}, and R³Sb, when present, is independently selected from hydrogen, methyl, ethyl, n-propyl, and isopropyl. In an even further aspect, each occurrence of R³¹, R³², R³⁴, R^{35a}, and R^{35b}, when present, is independently selected from hydrogen, methyl, and ethyl. In a still further aspect, each occurrence of R³¹, R³², R³⁴, R^{35a}, and R^{35b}, when present, is independently selected from hydrogen and ethyl. In yet a further aspect, each occurrence of R³¹, R³², R³⁴, R^{35a}, and R^{35b}, when present, is independently selected from hydrogen and methyl.

In a further aspect, each occurrence of R³¹, R³², R³⁴, R^{35a}, and R^{35b}, when present, is independently selected from C1-C12 alkyl. In a still further aspect, each occurrence of R³¹, R³², R³⁴, R^{35a}, and R^{35b}, when present, is independently selected from C1-C6 alkyl. In yet a further aspect, each occurrence of R³¹, R³², R³⁴, R^{35a}, and R^{35b}, when present, is independently selected from C1-C4 alkyl. In an even further aspect, each occurrence of R³¹, R³², R³⁴, R^{35a}, and R^{35b}, when present, is independently selected from methyl, ethyl, n-propyl, and isopropyl. In a still further aspect, each occurrence of R³¹, R³², R³⁴, R^{35a}, and R^{35b}, when present, is independently selected from methyl and ethyl. In yet a further aspect, each occurrence of R³¹, R³, R³⁴, R^{35a}, and R^{35b}, when present, is ethyl. In an even further aspect, each occurrence of R³¹, R³², R³⁴, R^{35b}, and R^{35b}, when present, is methyl.

In a further aspect, each occurrence of R³¹, R³², R³⁴, R^{35a}, and R^{35b}, when present, is hydrogen.

t. R³³ Groups

In one aspect, each occurrence of R³³, when present, is independently selected from hydrogen, C1-C12 alkyl, and monocyclic aryl monosubstituted with a methyl group. In a further aspect, each occurrence of R³³, when present, is independently selected from hydrogen, C1-C8 alkyl, and monocyclic aryl monosubstituted with a methyl group. In a still further aspect, each occurrence of R³³, when present, is independently selected from hydrogen, C1-C4 alkyl, and monocyclic aryl monosubstituted with a methyl group. In yet a further aspect, each occurrence of R³³, when present, is independently selected from hydrogen, methyl, ethyl, n-propyl, isopropyl, and monocyclic aryl monosubstituted with a methyl group. In an even further aspect, each occurrence of R³³, when present, is independently selected from hydrogen, methyl, ethyl, and monocyclic aryl monosubstituted with a methyl group. In a still further aspect, each occurrence of R³³, when present, is independently selected from hydrogen, methyl, and monocyclic aryl monosubstituted with a methyl group.

In various aspects, each occurrence of R³³, when present, is independently selected from hydrogen and C1-C12 alkyl. In a further aspect, each occurrence of R³³, when present, is independently selected from hydrogen and C1-C8 alkyl. In a still further aspect, each occurrence of R³³, when present, is independently selected from hydrogen and C1-C4 alkyl. In yet a further aspect, each occurrence of R³³, when present, is independently selected from hydrogen, methyl, ethyl, n-propyl, and isopropyl. In an even further aspect, each occurrence of R³³, when present, is independently selected from hydrogen, methyl, and ethyl. In a still further aspect, each occurrence of R³³, when present, is independently selected from hydrogen and ethyl. In yet a further aspect, each occurrence of R³³, when present, is independently selected from hydrogen and methyl.

In a further aspect, each occurrence of R³³, when present, is independently selected from hydrogen and monocyclic aryl monosubstituted with a methyl group.

In a further aspect, each occurrence of R³³, when present, is hydrogen.

u. R³⁶ Groups

In one aspect, each occurrence of R³⁶, when present, is independently selected from hydrogen and C1-C12 alkyl. In a further aspect, each occurrence of R³⁶, when present, is independently selected from hydrogen and C1-C6 alkyl. In a still further aspect, each occurrence of R³⁶, when present, is independently selected from hydrogen and C1-C4 alkyl. In yet a further aspect, each occurrence of R³⁶, when present, is independently selected from hydrogen, methyl, ethyl, n-propyl, and isopropyl. In an even further aspect, each occurrence of R³⁶, when present, is independently selected from hydrogen, methyl, and ethyl. In a still further aspect, each occurrence of R³⁶, when present, is independently selected from hydrogen and ethyl. In yet a further aspect, each occurrence of R³⁶, when present, is independently selected from hydrogen and methyl.

In a further aspect, each occurrence of R³⁶, when present, is independently selected from C1-C12 alkyl. In a still further aspect, each occurrence of R³⁶, when present, is independently selected from C1-C6 alkyl. In yet a further aspect, each occurrence of R³⁶, when present, is independently selected from C1-C4 alkyl. In an even further aspect, each occurrence of R³⁶, when present, is independently selected from methyl, ethyl, n-propyl, and isopropyl. In a still further aspect, each occurrence of R³⁶, when present, is independently selected from methyl and ethyl. In yet a further aspect, each occurrence of R³⁶, when present, is ethyl. In an even further aspect, each occurrence of R³⁶, when present, is methyl.

In a further aspect, each occurrence of R³⁶, when present, is hydrogen.

v. R³⁷ Groups

In one aspect, R³⁷, when present, is selected from hydrogen, C1-C4 alkyl, —SO₂R⁵¹, and a structure having a formula:

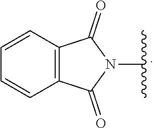 [SMILES: *N1C(=O)c2ccccc2C1=O]

In a further aspect, R³⁷, when present, is selected from hydrogen and C1-C4 alkyl. In a still further aspect, R³⁷, when present, is selected from hydrogen, methyl, ethyl, n-propyl, and isopropyl. In yet a further aspect, R³⁷, when present, is selected from hydrogen, methyl, and ethyl. In an even further aspect, R³⁷, when present, is selected from hydrogen and ethyl. In a still further aspect, R³⁷, when present, is selected from hydrogen and methyl.

In a further aspect, R³⁷, when present, is C1-C4 alkyl. In a still further aspect, R³⁷, when present, is selected from methyl, ethyl, n-propyl, and isopropyl. In yet a further aspect, R³⁷, when present, is selected from methyl and ethyl. In an even further aspect, R³⁷, when present, is ethyl. In a still further aspect, R³⁷, when present, is methyl.

In a further aspect, R³⁷, when present, is hydrogen.

In a further aspect, R³⁷, when present, is selected from hydrogen and —SO₂R⁵¹. In a still further aspect, R³⁷, when present, is —SO₂R⁵¹.

In a further aspect, R³⁷, when present, is a structure having a formula:

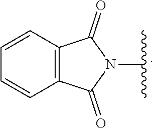 [SMILES: *N1C(=O)c2ccccc2C1=O]

w. R⁴⁰ Groups

In one aspect, each occurrence of R⁴⁰, when present, is independently a C1-C30 alkyl functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide. In a further aspect, each occurrence of R⁴⁰, when present, is independently a C1-C15 alkyl functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide. In a still further aspect, each occurrence of R⁴⁰, when present, is independently a C1-C8 alkyl functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide. In yet a further aspect, each occurrence of R⁴⁰, when present, is independently a C1-C4 alkyl functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide. In an even further aspect, each occurrence of R⁴⁰, when present, is independently a methyl, ethyl, n-propyl, or isopropyl and is functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide. In a still further aspect, each occurrence of R⁴⁰, when present, is independently a methyl or ethyl and is functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide. In yet a further aspect, each occurrence of R⁴⁰, when present, is independently an ethyl functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide. In an even further aspect, each occurrence of R⁴⁰, when present, is independently a methyl functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide.

In one aspect, each occurrence of R⁴⁰, when present, is independently a C1-C30 alkyl functionalized with a group selected from —SH, —NH₂, —NHOH, terminal alkyne, haloacetyl, maleimide, isothiocyanate, N-hydroxysuccinimde, succinimidyl ester, tetrafluorophenyl ester, sulfodichlorophenol ester, and hydrazide. In a further aspect, each occurrence of R⁴⁰, when present, is independently a C1-C15 alkyl functionalized with a group selected from —SH, —NH₂, —NHOH, terminal alkyne, haloacetyl, maleimide, isothiocyanate, N-hydroxysuccinimde, succinimidyl ester, tetrafluorophenyl ester, sulfodichlorophenol ester, and hydrazide. In a still further aspect, each occurrence of R⁴⁰, when present, is independently a C1-C8 alkyl functionalized with a group selected from —SH, —NH₂, —NHOH, terminal alkyne, haloacetyl, maleimide, isothiocyanate, N-hydroxysuccinimde, succinimidyl ester, tetrafluorophenyl ester, sulfodichlorophenol ester, and hydrazide. In yet a further aspect, each occurrence of R⁴⁰, when present, is independently a C1-C4 alkyl functionalized with a group selected from —SH, —NH₂, —NHOH, terminal alkyne, haloacetyl, maleimide, isothiocyanate, N-hydroxysuccinimde, succinimidyl ester, tetrafluorophenyl ester, sulfodichlorophenol ester, and hydrazide. In an even further aspect, each occurrence of R⁴⁰, when present, is independently a methyl, ethyl, n-propyl, or isopropyl and is functionalized with a group selected from —SH, —NH₂, —NHOH, terminal alkyne, haloacetyl, maleimide, isothiocyanate, N-hydroxysuccinimde, succinimidyl ester, tetrafluorophenyl ester, sulfodichlorophenol ester, and hydrazide. In a still further aspect, each occurrence of R⁴⁰, when present, is independently a methyl or ethyl and is functionalized with a group selected from —SH, —NH₂, —NHOH, terminal alkyne, haloacetyl, maleimide, isothiocyanate, N-hydroxysuccinimde, succinimidyl ester, tetrafluorophenyl ester, sulfodichlorophenol ester, and hydrazide. In yet a further aspect, each occurrence of R⁴⁰, when present, is independently an ethyl functionalized with a group selected from —SH, —NH₂, —NHOH, terminal alkyne, haloacetyl, maleimide, isothiocyanate, N-hydroxysuccinimde, succinimidyl ester, tetrafluorophenyl ester, sulfodichlorophenol ester, and hydrazide. In an even further aspect, each occurrence of R⁴⁰, when present, is independently a methyl functionalized with a group selected from —SH, —NH₂, —NHOH, terminal alkyne, haloacetyl, maleimide, isothiocyanate, N-hydroxysuccinimde, succinimidyl ester, tetrafluorophenyl ester, sulfodichlorophenol ester, and hydrazide.

In a further aspect, R⁴⁰ is a C1-C30 alkyl functionalized with a maleimide group. In a still further aspect, R⁴⁰ is a C1-C15 alkyl functionalized with a maleimide group. In yet a further aspect, R⁴⁰ is a C1-C8 alkyl functionalized with a maleimide group. In an even further aspect, R⁴⁰ is a C1-C4 alkyl functionalized with a maleimide group. In a still further aspect, R⁴⁰ is selected from the group consisting of methyl, ethyl, n-propyl, and isopropyl and is functionalized with a maleimide group. In yet a further aspect, R⁴⁰ is selected from the group consisting of methyl and ethyl and is functionalized with a maleimide group. In an even further aspect, R⁴⁰ is ethyl functionalized with a maleimide group. In a still further aspect, R⁴⁰ is methyl functionalized with a maleimide group.

In a further aspect, R⁴⁰ is a structure:

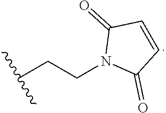 [SMILES: *CCN1C(=O)C=CC1=O]

In a further aspect, R⁴⁰ is functionalized with an ester selected from a succinimidyl ester, a tetrafluorophenyl ester, and a sulfodichlorophenol ester.

x. R⁴¹ and R⁴² Groups

In one aspect, each occurrence of R⁴¹ and R⁴², when present, is independently selected from hydrogen and C1-C12 alkyl. In a further aspect, each occurrence of R⁴¹ and R⁴², when present, is independently selected from hydrogen and C1-C6 alkyl. In a still further aspect, each occurrence of R⁴¹ and R⁴², when present, is independently selected from hydrogen and C1-C4 alkyl. In yet a further aspect, each occurrence of R⁴¹ and R⁴², when present, is independently selected from hydrogen, methyl, ethyl, n-propyl, and isopropyl. In an even further aspect, each occurrence of R⁴¹ and R⁴², when present, is independently selected from hydrogen, methyl, and ethyl. In a still further aspect, each occurrence of R⁴¹ and R⁴², when present, is independently selected from hydrogen and ethyl. In yet a further aspect, each occurrence of R⁴¹ and R⁴², when present, is independently selected from hydrogen and methyl.

In a further aspect, each occurrence of R⁴¹ and R⁴², when present, is independently selected from C1-C12 alkyl. In a still further aspect, each occurrence of R⁴¹ and R⁴², when present, is independently selected from C1-C6 alkyl. In yet a further aspect, each occurrence of R⁴¹ and R⁴², when present, is independently selected from C1-C4 alkyl. In an even further aspect, each occurrence of R⁴¹ and R⁴², when present, is independently selected from methyl, ethyl, n-propyl, and isopropyl. In a still further aspect, each occurrence of R⁴¹ and R⁴², when present, is independently selected from methyl and ethyl. In yet a further aspect, each occurrence of R⁴¹ and R⁴², when present, is ethyl. In an even further aspect, each occurrence of R⁴¹ and R⁴², when present, is methyl.

In a further aspect, each occurrence of R⁴¹ and R⁴², when present, is hydrogen.

y. R^{50a}, R^{50b}, R^{50c}, And R^{50d} Groups

In one aspect, each of R^{50a}, R^{50b}, R^{50c}, and R^{50d}, when present, is independently selected from hydrogen, —F, and —Cl. In a further aspect, each of R₅₀ₐ, R^{50b}, R^{50c}, and R^{50d}, when present, is independently selected from hydrogen and —F. In a still further aspect, each of R^{50a}, R^{50b}, R_{50c}, and R^{50d}, when present, is independently selected from hydrogen and —Cl. In yet a further aspect, each of R^{50a}, R_{50b}, R^{50c}, and R^{50d}, when present, is hydrogen.

In a further aspect, each of R^{50a}, R^{50b}, R^{50c}, and R^{50d}, when present, is independently selected from —F and —Cl. In a still further aspect, each of R^{50a}, R^{50b}, R^{50c}, and R^{50d}, when present, is —F. In yet a further aspect, each of R^{50a}, R^{50b}, R^{50c}, and R^{50d}, when present, is —Cl.

z. R^{51a} and R^{51b} Groups

In one aspect, each of R^{51a} and R^{51b}, when present, is independently selected from hydrogen and —C(O)(C1-C12 alkyl). In a further aspect, each of R^{51a} and R^{51b}, when present, is independently selected from hydrogen and —C(O)(C1-C6 alkyl). In a still further aspect, each of R^{51a} and R^{51b}, when present, is independently selected from hydrogen and —C(O)(C1-C4 alkyl). In yet a further aspect, each of R^{51a} and R^{51b}, when present, is independently selected from hydrogen, —C(O)CH₃, —C(O)CH₂CH₃, —C(O)CH₂CH₂CH₃, and —C(O)CH(CH₃)₂. In a still further aspect, each of R^{51a} and R^{51b}, when present, is independently selected from hydrogen, —C(O)CH₃, and —C(O)CH₂CH₃. In yet a further aspect, each of R^{51a} and R^{51b}, when present, is independently selected from hydrogen and —C(O)CH₂CH₃. In a still further aspect, each of R^{15a} and R^{51b}, when present, is independently selected from hydrogen and —C(O)CH₃. In yet a further aspect, each of R^{51a} and R^{51b}, when present, is hydrogen.

aa. R^{52a}, R^{52b}, R^{52c}, and R^{52d} Groups

In one aspect, each of R^{52a}, R^{52b}, R^{52c}, and R^{52d}, when present, is independently selected from hydrogen, —F, and —Cl. In a further aspect, each of R^{52a}, R^{52b}, R^{52c}, and R^{52d}, when present, is independently selected from hydrogen and —F. In a still further aspect, each of R^{52a}, R^{52b}, R^{52c}, and R^{52d}, when present, is independently selected from hydrogen and —Cl. In yet a further aspect, each of R^{52a}, R^{52b}, R^{52c}, and R^{52d}, when present, is hydrogen.

In a further aspect, each of R^{52a}, R^{52b}, R^{52c}, and R^{52d}, when present, is independently selected from —F and —Cl. In a still further aspect, each of R^{52a}, R^{52b}, R^{52c}, and R^{52d}, when present, is —F. In yet a further aspect, each of R^{52a}, R^{52b}, R_{52c}, and R^{52d}, when present, is —Cl.

bb. R⁶¹ and R⁶² Groups

In one aspect, each of R⁶¹ and R⁶², when present, is independently selected from hydrogen and C1-C12 alkyl. In a further aspect, each of R61 and R⁶², when present, is independently selected from hydrogen and C1-C6 alkyl. In a still further aspect, each of R⁶¹ and R⁶², when present, is independently selected from hydrogen and C1-C4 alkyl. In yet a further aspect, each of R⁶¹ and R⁶², when present, is independently selected from hydrogen, methyl, ethyl, n-propyl, and isopropyl. In an even further aspect, each of R⁶¹ and R⁶², when present, is independently selected from hydrogen, methyl, and ethyl. In a still further aspect, each of R⁶¹ and R⁶², when present, is independently selected from hydrogen and ethyl. In yet a further aspect, each of R⁶¹ and R⁶², when present, is independently selected from hydrogen and methyl.

In a further aspect, each of R⁶¹ and R⁶², when present, is independently selected from C1-C12 alkyl. In a still further aspect, each of R⁶¹ and R⁶², when present, is independently selected from C1-C6 alkyl. In yet a further aspect, each of R⁶¹ and R⁶², when present, is independently selected from C1-C4 alkyl. In an even further aspect, each of R⁶¹ and R⁶², when present, is independently selected from methyl, ethyl, n-propyl, and isopropyl. In a still further aspect, each of R⁶¹ and R⁶², when present, is independently selected from methyl and ethyl. In yet a further aspect, each of R⁶¹ and R⁶², when present, is ethyl. In an even further aspect, each of R⁶¹ and R⁶², when present, is methyl.

In a further aspect, each of R⁶¹ and R⁶², when present, is hydrogen.

cc. R⁷¹ Groups

In one aspect, R⁷¹, when present, is selected from hydrogen, C1-C4 alkyl, —CH₂CH₂Si(CH₃)₃, and monocyclic aryl monosubstituted with a methyl group. In a further aspect, R⁷¹, when present, is selected from hydrogen, methyl, ethyl, n-propyl, isopropyl, —CH₂CH₂Si(CH₃)₃, and monocyclic aryl monosubstituted with a methyl group. In a still further aspect, R⁷¹, when present, is selected from hydrogen, methyl, ethyl, —CH₂CH₂Si(CH₃)₃, and monocyclic aryl monosubstituted with a methyl group. In yet a further aspect, R⁷¹, when present, is selected from hydrogen, methyl, —CH₂CH₂Si(CH₃)₃, and monocyclic aryl monosubstituted with a methyl group.

In various aspects, R⁷¹, when present, is selected from hydrogen and C1-C4 alkyl. In a further aspect, R⁷¹, when present, is selected from hydrogen, methyl, ethyl, n-propyl, and isopropyl. In a still further aspect, R⁷¹, when present, is selected from hydrogen, methyl, and ethyl. In yet a further aspect, R⁷¹, when present, is selected from hydrogen and methyl.

In various aspects, R⁷¹, when present, is C1-C4 alkyl. In a further aspect, R⁷¹, when present, is selected from methyl, ethyl, n-propyl, and isopropyl. In a still further aspect, R⁷¹, when present, is selected from methyl and ethyl. In yet a further aspect, R⁷¹, when present, is methyl.

In a further aspect, R⁷¹, when present, is selected from hydrogen and —CH₂CH₂Si(CH₃)₃. In a still further aspect, R⁷¹, when present, is —CH₂CH₂Si(CH₃)₃.

In a further aspect, R⁷¹, when present, is selected from hydrogen and monocyclic aryl monosubstituted with a methyl group. In a still further aspect, R⁷¹, when present, is monocyclic aryl monosubstituted with a methyl group.

In a further aspect, R⁷¹, when present, is hydrogen.

dd. Cy¹ Groups

In one aspect, each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. Examples of heterocycloalkyls include, but are not limited to, aziridinyl, pyrrolidinyl, pyrrolidino, piperidinyl, piperidino, piperazinyl, piperazino, morpholinyl, morpholino, thiomorpholinyl, thiomorpholino, tetrahydrofuranyl, tetrahydrothiofuranyl, tetrahydropyranyl, and pyranyl. In a further aspect, each occurrence of Cy¹, when present, is heterocycloalkyl substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, each occurrence of Cy¹, when present, is heterocycloalkyl substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, each occurrence of Cy¹, when present, is heterocycloalkyl monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, each occurrence of Cy¹, when present, is unsubstituted heterocycloalkyl.

Ee. Ar¹ Groups

In one aspect, each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a further aspect, each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, or 2 groups independently selected from halogen, —OH, —NH₂, C1-C4 alkoxy, C1-C4 hydroxy, C1-C4 aminoalkyl, CT-C4 alkylamino, and (C1-C4)(C1-C4) dialkylamino. In a still further aspect, each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and is monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and is unsubstituted.

In various aspects, each occurrence of Ar¹, when present, is monocyclic aryl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a further aspect, each occurrence of Ar¹, when present, is monocyclic aryl substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, each occurrence of Ar¹, when present, is monocyclic aryl substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, each occurrence of Ar¹, when present, is monocyclic aryl monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, each occurrence of Ar¹, when present, is unsubstituted monocyclic aryl.

In various aspects, each occurrence of Ar¹, when present, is selected from morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a further aspect, each occurrence of Ar¹, when present, is selected from morpholinyl, anilinyl, indolyl, pyrrolyl, imidazolyl, benzimidazolyl, pyrazolyl, guanidinyl, and piperazinyl and substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, each occurrence of Ar¹, when present, is selected from morpholinyl, anilinyl, indolyl, pyrrolyl, imidazolyl, benzimidazolyl, pyrazolyl, guanidinyl, and piperazinyl and substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, each occurrence of Ar¹, when present, is selected from morpholinyl, anilinyl, indolyl, pyrrolyl, imidazolyl, benzimidazolyl, pyrazolyl, guanidinyl, and piperazinyl and is monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, each occurrence of Ar¹, when present, is selected from morpholinyl, anilinyl, indolyl, pyrrolyl, imidazolyl, benzimidazolyl, pyrazolyl, guanidinyl, and piperazinyl and is unsubstituted.

Ff. Ar² Groups

In one aspect, each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In a further aspect, each occurrence of Ar², when present, is independently selected from aryl and heteroaryl, and is substituted with 0, 1, or 2 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In a still further aspect, each occurrence of Ar², when present, is independently selected from aryl and heteroaryl, and is substituted with 0 or 1 group selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In yet a further aspect, each occurrence of Ar², when present, is independently selected from aryl and heteroaryl, and is monosubstituted with a group selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In an even further aspect, each occurrence of Ar², when present, is independently selected from aryl and heteroaryl, and is unsubstituted.

In various aspects, each occurrence of Ar², when present, is independently selected from aryl substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In a further aspect, each occurrence of Ar², when present, is independently selected from aryl substituted with 0, 1, or 2 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In a still further aspect, each occurrence of Ar², when present, is independently selected from aryl substituted with 0 or 1 group selected from halogen, —OH, —NH₂, C1-C4 alkoxy, C1-C4 hydroxy, C1-C4 aminoalkyl, CT-C4 alkylamino, (C1-C4)(C1-C4) dialkylamino, —(C1-C12 alkyl)Ar⁴, and Ar⁴. In yet a further aspect, each occurrence of Ar², when present, is independently selected from aryl monosubstituted with a group selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In an even further aspect, each occurrence of Ar², when present, is unsubstituted aryl.

In various aspects, each occurrence of Ar², when present, is independently selected from phenyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In a further aspect, each occurrence of Ar², when present, is independently selected from phenyl substituted with 0, 1, or 2 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In a still further aspect, each occurrence of Ar², when present, is independently selected from phenyl substituted with 0 or 1 group selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In yet a further aspect, each occurrence of Ar², when present, is independently selected from phenyl monosubstituted with a group selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In an even further aspect, each occurrence of Ar², when present, is unsubstituted phenyl.

In various aspects, each occurrence of Ar², when present, is independently selected from heteroaryl substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. Examples of heteroaryls include, but are not limited to, pyrrolyl, furanyl, thiophenyl, indolyl, benzofuranyl, benzothiophenyl, triazolyl, imidazolyl, oxazolyl, thiazolyl, pyrazolyl, isoxazolyl, isothiazolyl, pyridinyl, quinolinyl, and isoquinolinyl. In a further aspect, each occurrence of Ar², when present, is independently selected from heteroaryl substituted with 0, 1, or 2 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In a still further aspect, each occurrence of Ar², when present, is independently selected from heteroaryl substituted with 0 or 1 group selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In yet a further aspect, each occurrence of Ar², when present, is independently selected from heteroaryl monosubstituted with a group selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴. In an even further aspect, each occurrence of Ar², when present, is unsubstituted heteroaryl.

In a further aspect, each occurrence of Ar², when present, is triazolyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar³, and Ar³. In a still further aspect, each occurrence of Ar², when present, is triazolyl substituted with 0, 1, or 2 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar³, and Ar³. In yet a further aspect, each occurrence of Ar², when present, is triazolyl substituted with 0 or 1 group selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar³, and Ar³. In an even further aspect, each occurrence of Ar², when present, is triazolyl monosubstituted with a group selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar³, and Ar³. In a still further aspect, each occurrence of Ar², when present, is unsubstituted triazolyl.

In a further aspect, each occurrence of Ar², when present, is triazolyl substituted with 0, 1, 2, or 3 groups independently selected from —(C1-C12 alkyl)Ar³ and Ar³. In a still further aspect, each occurrence of Ar², when present, is triazolyl substituted with 0, 1, or 2 groups independently selected from —(C1-C12 alkyl)Ar³ and Ar³. In yet a further aspect, each occurrence of Ar², when present, is triazolyl substituted with 0 or 1 group selected from —(C1-C12 alkyl)Ar³ and Ar³. In an even further aspect, each occurrence of Ar², when present, is triazolyl monosubstituted with a group selected from —(C1-C12 alkyl)Ar³ and Ar³.

In a further aspect, each occurrence of Ar², when present, is triazolyl substituted with 1 Ar³ group.

Gg. Ar³ Groups

In one aspect, each occurrence of Ar³, when present, is a structure represented by a formula selected from:

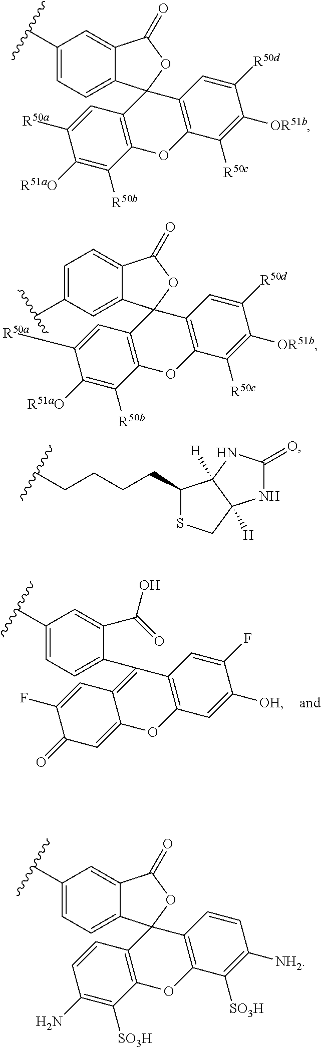

In a further aspect, each occurrence of Ar³, when present, is a structure represented by a formula selected from:

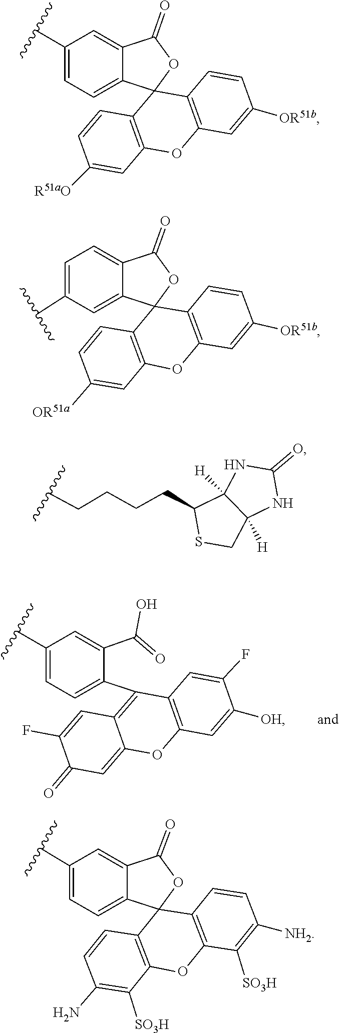

In a further aspect, each occurrence of Ar³, when present, is a structure represented by a formula:

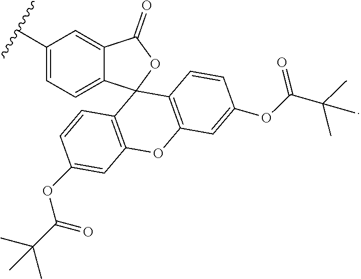 [SMILES: *c1ccc2c(c1)C(=O)OC21c2ccc(OC(=O)C(C)(C)C)cc2Oc2cc(OC(=O)C(C)(C)C)ccc21]

hh. Ar⁴ Groups

In one aspect, each occurrence of Ar⁴, when present, is a structure represented by a formula selected from:

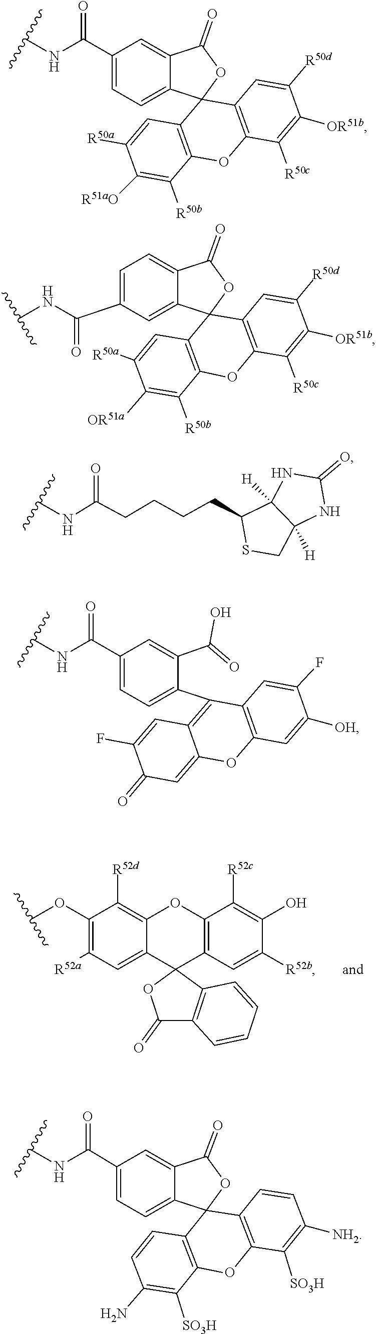

In a further aspect, In one aspect, each occurrence of Ar⁴, when present, is a structure represented by a formula selected from:

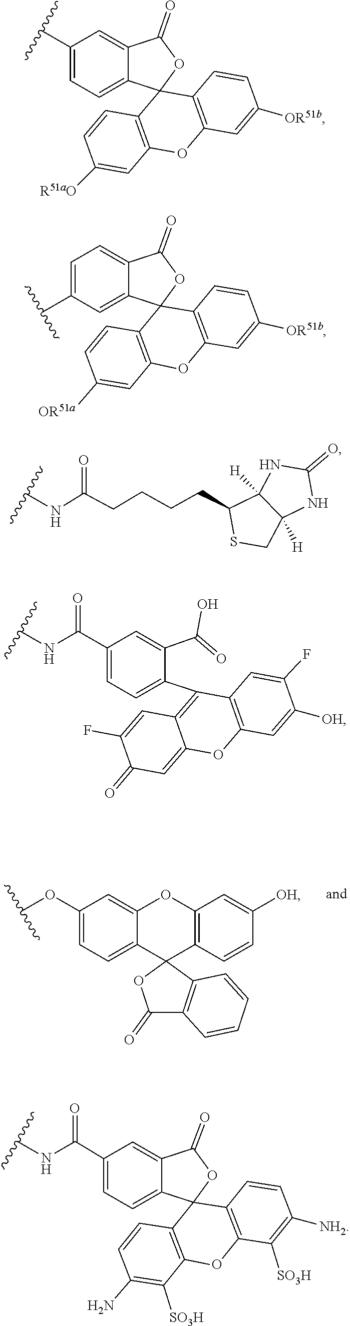

In a further aspect, each occurrence of Ar⁴, when present, is a structure represented by a formula:

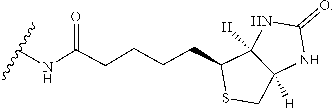 [SMILES: *NC(=O)CCCC[C@@H]1SC[C@@H]2NC(=O)N[C@@H]21]

In a further aspect, each occurrence of Ar⁴, when present, is a structure represented by a formula:

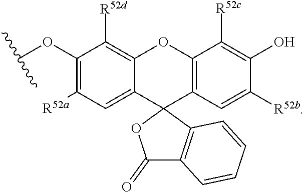

In a further aspect, each occurrence of Ar⁴, when present, is a structure represented by a formula:

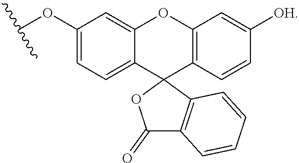 [SMILES: *Oc1ccc2c(c1)Oc1cc(O)ccc1C21OC(=O)c2ccccc21]

II. Ar⁵ Groups

In one aspect, Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a further aspect, Ar⁵, when present, is selected from monocyclic 6-membered aryl and anthracene-9,10-dionyl, and is substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, Ar⁵, when present, is selected from monocyclic 6-membered aryl and anthracene-9,10-dionyl, and is substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, Ar⁵, when present, is selected from monocyclic 6-membered aryl and anthracene-9,10-dionyl, and is monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, Ar⁵, when present, is selected from monocyclic 6-membered aryl and anthracene-9,10-dionyl, and is unsubstituted.

In various aspects, Ar⁵, when present, is monocyclic 6-membered aryl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a further aspect, Ar⁵, when present, is monocyclic 6-membered aryl substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, Ar⁵, when present, is monocyclic 6-membered aryl substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, Ar⁵, when present, is monocyclic 6-membered aryl monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, Ar⁵, when present, is unsubstituted monocyclic 6-membered aryl.

In various aspects, Ar⁵, when present, is anthracene-9,10-dionyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a further aspect, Ar⁵, when present, is anthracene-9,10-dionyl substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, Ar⁵, when present, is anthracene-9,10-dionyl substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, Ar⁵, when present, is anthracene-9,10-dionyl monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, Ar⁵, when present, is unsubstituted anthracene-9,10-dionyl.

2. Example Compounds

In one aspect, a compound can be present as one or more of the following structures:

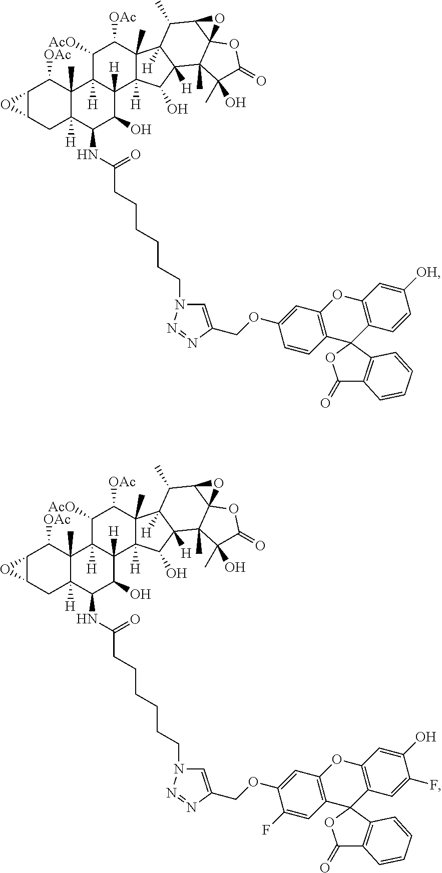

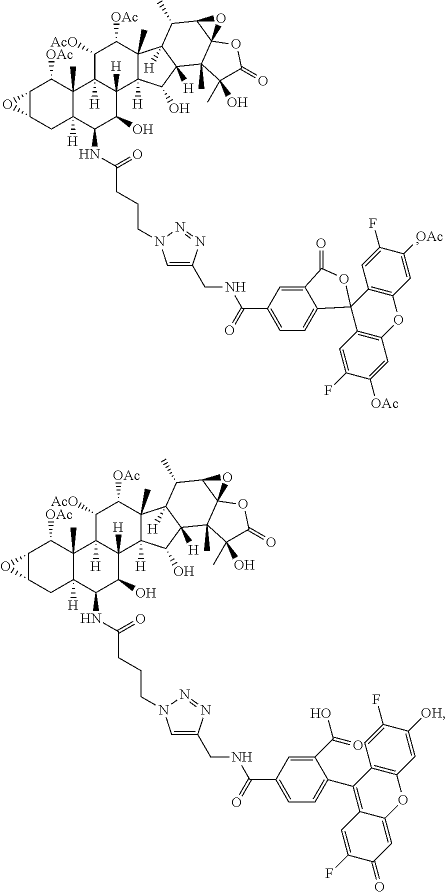

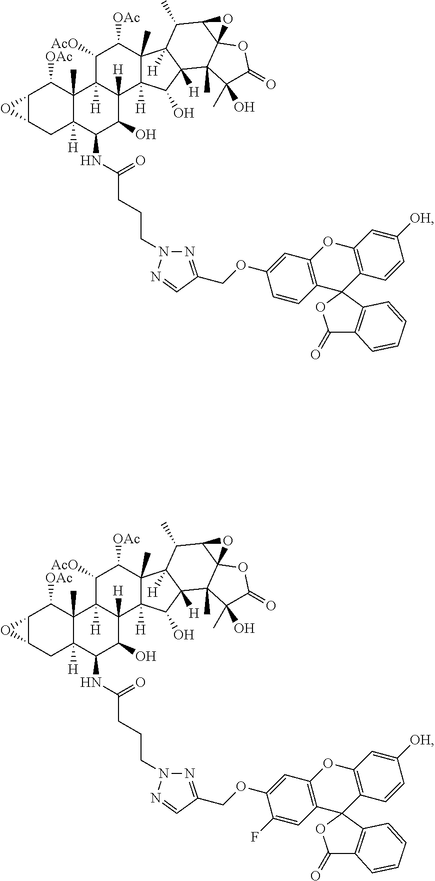

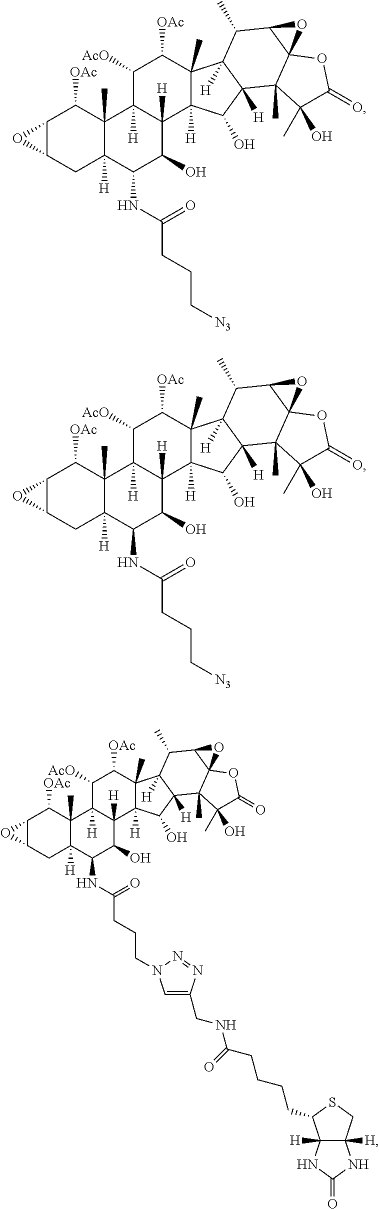

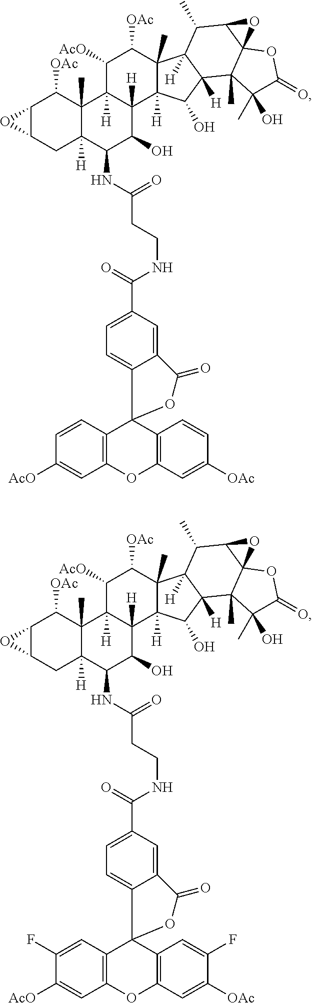

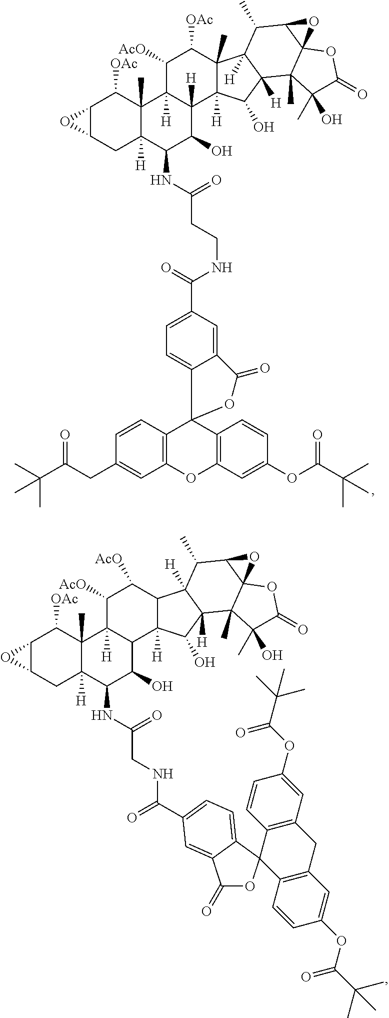

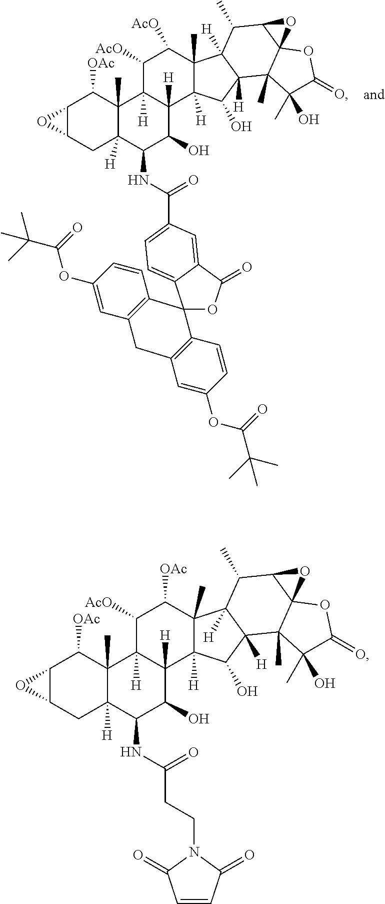

or a pharmaceutically acceptable salt thereof.

In one aspect, a compound can be present as one or more of the following structures:

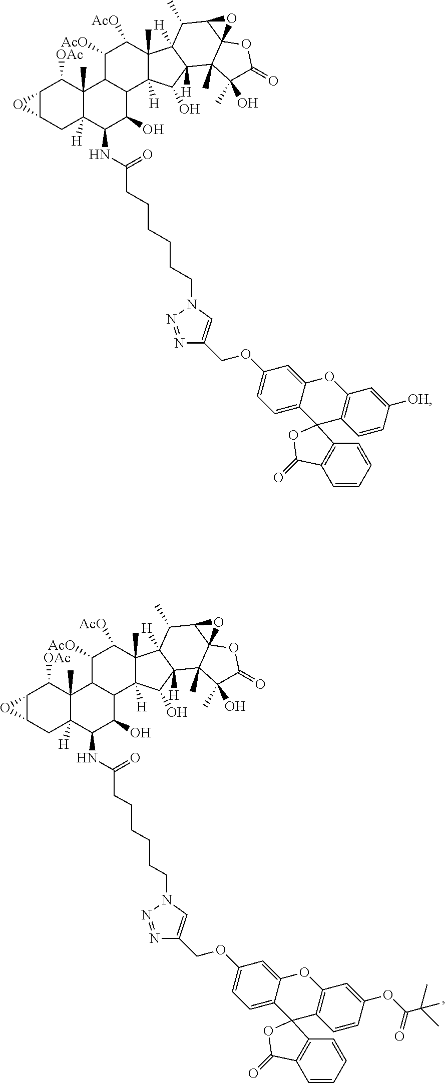 [SMILES: CC(=O)O[C@H]1C2C([C@@H](O)[C@@H](NC(=O)CCCCCCn3cc(COc4ccc5c(c4)Oc4cc(O)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@@H](NC(=O)CCCCCCn3cc(COc4ccc5c(c4)Oc4cc(OC(=O)C(C)(C)C)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

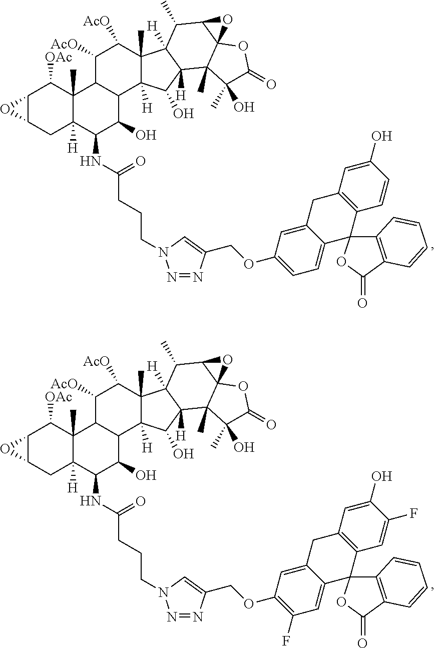 [SMILES: CC(=O)O[C@H]1C2C([C@@H](O)[C@@H](NC(=O)CCCn3cc(COc4ccc5c(c4)Cc4cc(O)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@@H](NC(=O)CCCn3cc(COc4cc5c(cc4F)C4(OC(=O)c6ccccc64)c4cc(F)c(O)cc4C5)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

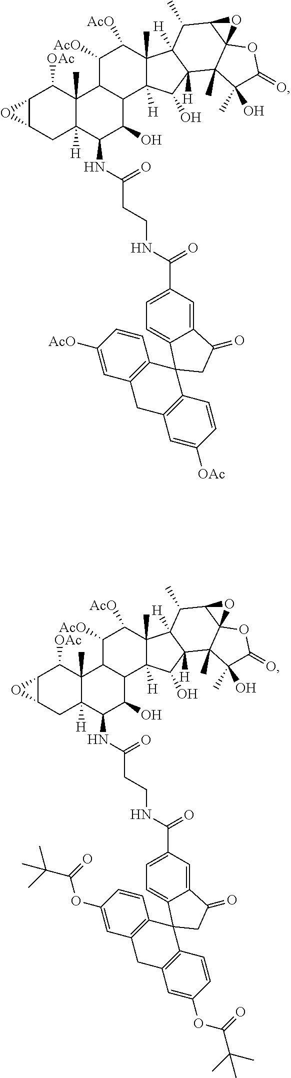

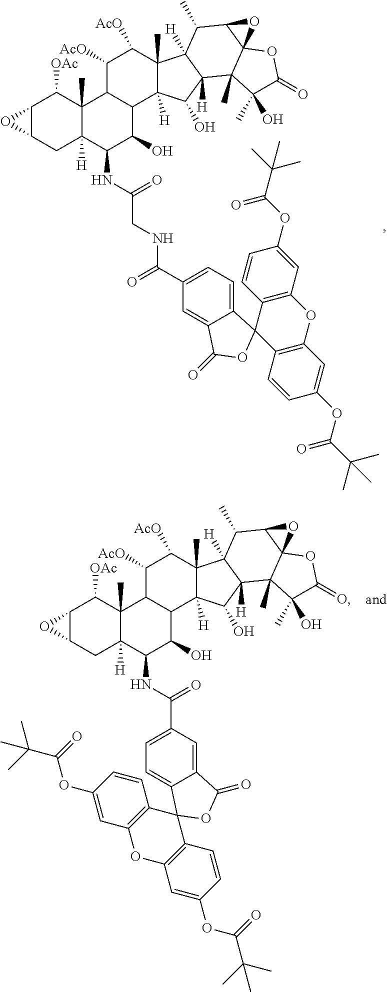

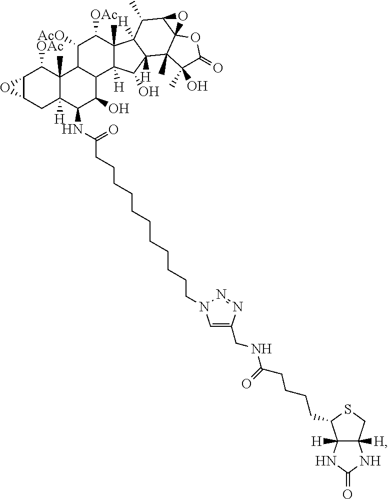

or a pharmaceutically acceptable salt thereof.

In one aspect, a compound can be present as one or more of the following structures:

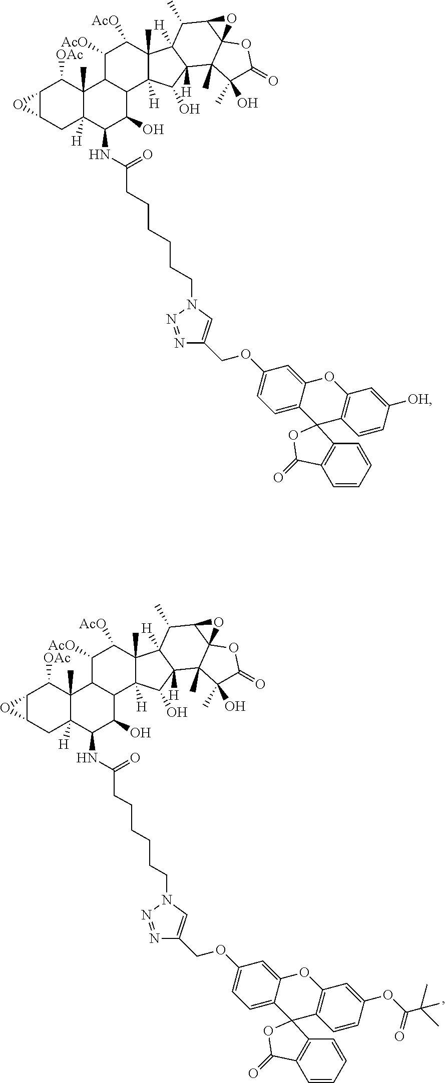 [SMILES: CC(=O)O[C@H]1C2C([C@@H](O)[C@@H](NC(=O)CCCCCCn3cc(COc4ccc5c(c4)Oc4cc(O)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@@H](NC(=O)CCCCCCn3cc(COc4ccc5c(c4)Oc4cc(OC(=O)C(C)(C)C)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

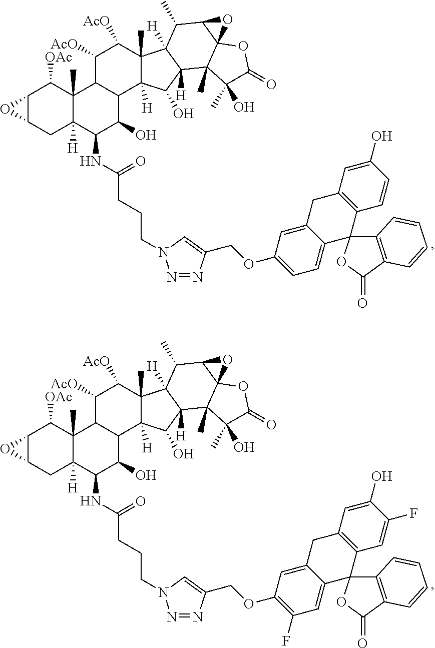 [SMILES: CC(=O)O[C@H]1C2C([C@@H](O)[C@@H](NC(=O)CCCn3cc(COc4ccc5c(c4)Cc4cc(O)ccc4C54OC(=O)c5ccccc54)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@@H](NC(=O)CCCn3cc(COc4cc5c(cc4F)C4(OC(=O)c6ccccc64)c4cc(F)c(O)cc4C5)nn3)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

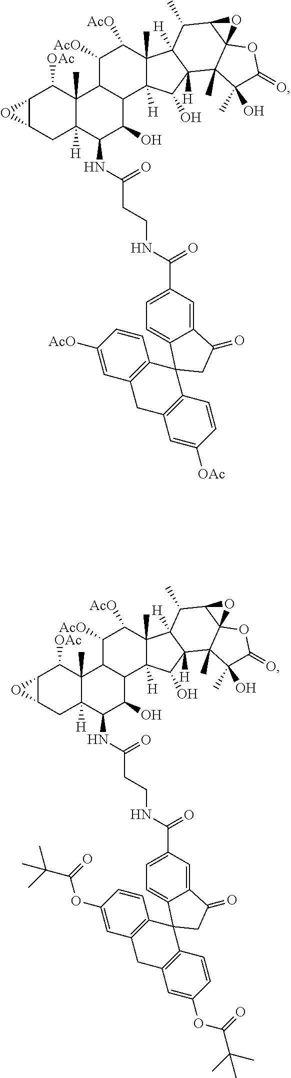

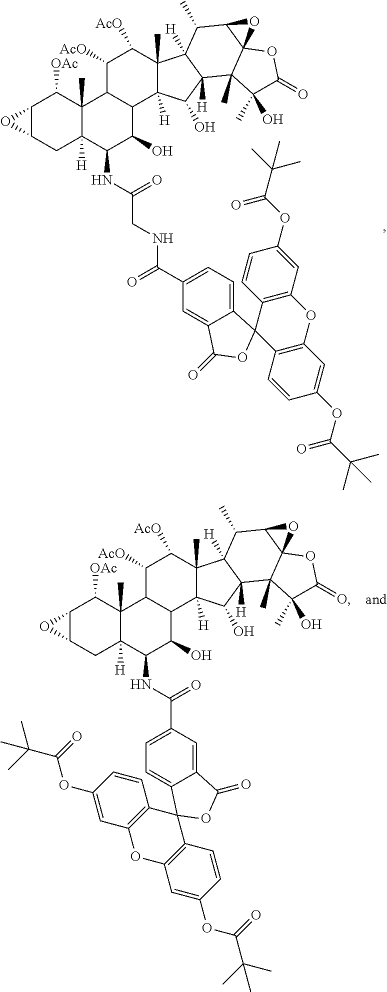

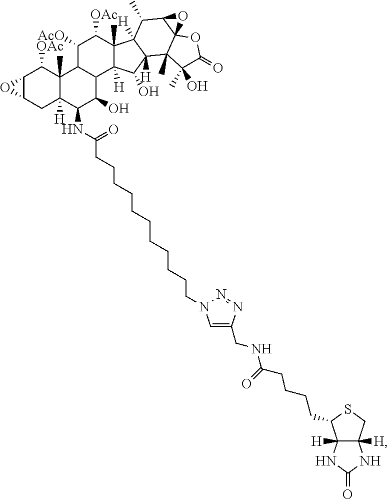

or a pharmaceutically acceptable salt thereof.

In one aspect, a compound can be present as:

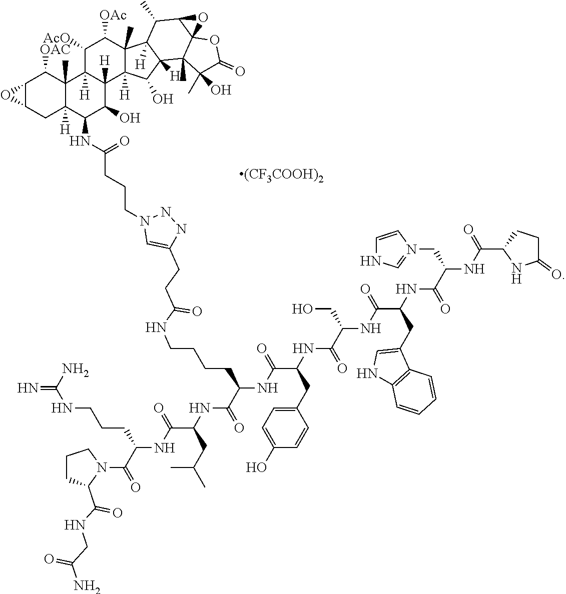

### C. Pharmaceutical Compositions

In one aspect, the invention relates to pharmaceutical compositions comprising a therapeutically effective amount at least one disclosed compound and a pharmaceutically acceptable carrier. In a further aspect, a pharmaceutical composition can be provided comprising a therapeutically effective amount of at least one disclosed compound. In a still further aspect, a pharmaceutical composition can be provided comprising a prophylactically effective amount of at least one disclosed compound. In yet a further aspect, the invention relates to pharmaceutical compositions comprising a pharmaceutically acceptable carrier and a compound, wherein the compound is present in an effective amount.

Thus, in one aspect, disclosed are pharmaceutical compositions comprising a therapeutically effective amount of a compound having a structure represented by a formula:

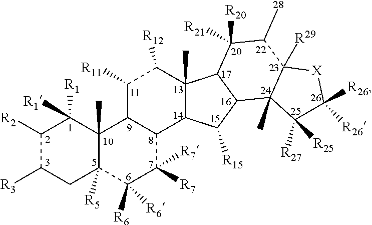

wherein each occurrence of - - - - - - is a single or double covalent bond; wherein X is selected from O, NR^{x}, and C(R^{x})₂; wherein each occurrence of R^{x}, when present, is independently selected from hydrogen and C1-C6 alkyl; wherein R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹; wherein each occurrence of R³¹, R³², R³⁴, R^{35a}, and R^{35b}, when present, is independently selected from hydrogen and C1-C12 alkyl; wherein each occurrence of R³³, when present, is independently selected from hydrogen, C1-C12 alkyl, and monocyclic aryl monosubstituted with a methyl group; wherein each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and wherein R^{1′} is hydrogen; or wherein each of R¹ and R^{1′} together comprise ═O or ═NR³⁶; wherein each occurrence of R³⁶, when present, is independently selected from hydrogen and C1-C12 alkyl; wherein each of R² and R³ is independently selected from hydrogen, —OH, C1-C12 hydroxy, and halogen; or wherein each of R₂ and R₃ together comprise —O—; wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; wherein each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, (C1-C30 alkyl)OC(O)NR^{35a}R^{35b}, Cy¹, Ar¹, —(C1-C30 alkyl)Ar¹, —OAr1, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)R⁴⁰, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰; wherein each occurrence of R⁴⁰, when present, is independently a C1-C30 alkyl functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide; wherein each occurrence of R⁴¹ and R⁴², when present, is independently selected from hydrogen and C1-C12 alkyl; wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴; wherein each occurrence of Ar⁴, when present, is a structure represented by a formula selected from:

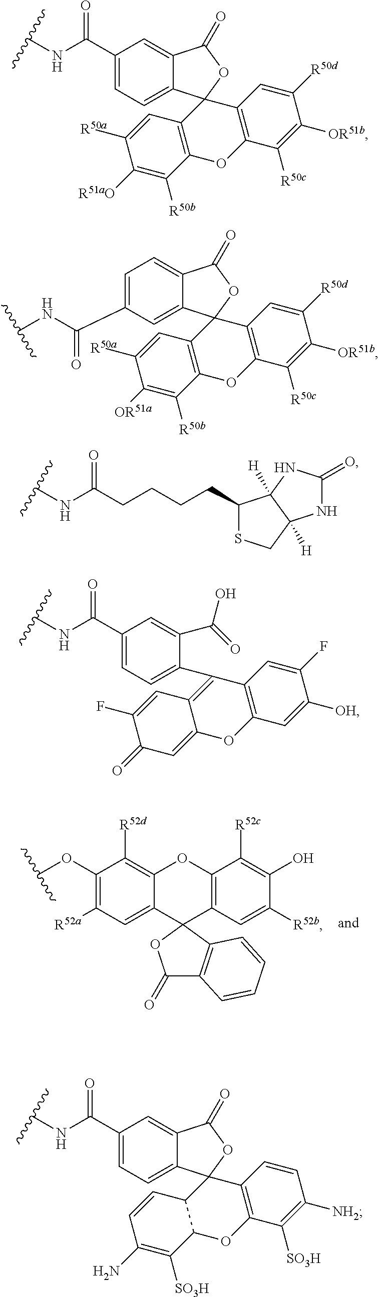

wherein each of R^{50a}, R^{50b}, R^{50c}, and R^{50d}, when present, is independently selected from hydrogen, —F, and —Cl; wherein each of R^{51a} and R^{51b}, when present, is independently selected from hydrogen and —C(O)(C1-C12 alkyl); wherein each of R^{52a}, R^{52b}, R^{52c}, and R^{52d}, when present, is independently selected from hydrogen, —F, and —Cl; wherein each occurrence of Ar³, when present, is a structure represented by a formula selected from:

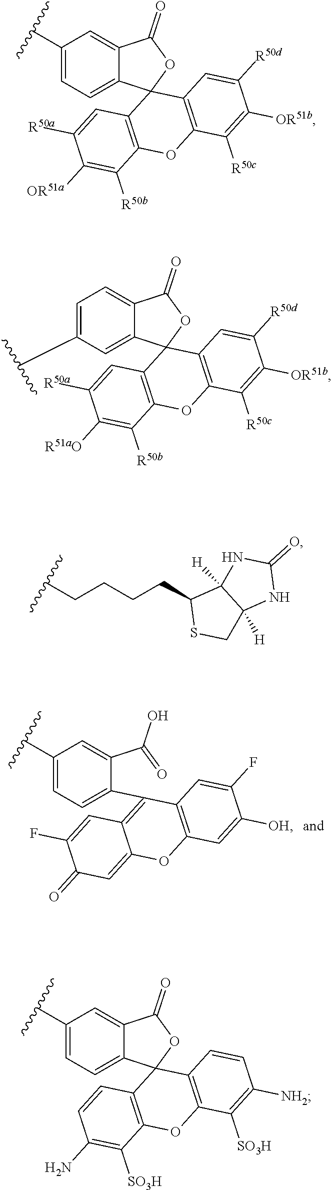

or wherein one of R⁶ and R^{6′} is absent; wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl); or wherein each of R⁷ and R^{7′} together comprise ═O; or wherein one of R⁷ and R^{7′} is absent; wherein each of R¹¹ and R¹² is independently selected from hydrogen, —OH, C1-C8 hydroxy, C1-C6 alkyl, C1-C8 alkoxy, and —OC(O)(C1-C8 alkyl); wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar2, —OC(O)(C1-C4 alkyl)Ar², and —OC(O)(C1-C8 azide); wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R²¹ is selected from hydrogen and C1-C6 alkyl; wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide); wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each of R²⁶ and R^{26′} is independently selected from hydrogen, —OH, C1-C8 hydroxy, and C1-C8 alkoxy; or wherein each of R²⁶ and R^{26′} together comprise ═O; wherein R²⁷ is selected from hydrogen and C1-C6 alkyl; wherein each of R²⁸ and R²⁹ is independently selected from hydrogen and halogen; or wherein each of R²⁸ and R²⁹ together comprise —O— or —N(R³⁷)—; wherein R³⁷, when present, is selected from hydrogen, C1-C4 alkyl, —SO₂R⁷¹, and a structure having a formula:

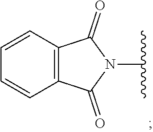 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that at least one of R⁶ and R^{6′} is —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, or —NR⁴¹C(O)R⁴⁰, and provided that when one or both of R²⁸ and R²⁹ is hydrogen then the occurrence of - - - - - - at C-22/C-23 is a double covalent bond, or a pharmaceutically acceptable salt thereof, and a pharmaceutically acceptable carrier.

In one aspect, disclosed are pharmaceutical compositions comprising a therapeutically effective of a compound having a structure represented by a formula:

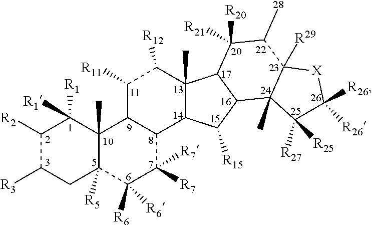

wherein each occurrence of - - - - - - is a single or double covalent bond; wherein X is selected from O, NR^{x} and C(R^{x})₂; wherein each occurrence of R^{x}, when present, is independently selected from hydrogen and C1-C6 alkyl; wherein R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹; wherein each occurrence of R³¹, R³², R³⁴, R^{35a}, and R^{35b}, when present, is independently selected from hydrogen and C1-C12 alkyl; wherein each occurrence of R³³, when present, is independently selected from hydrogen, C1-C12 alkyl, and monocyclic aryl monosubstituted with a methyl group; wherein each occurrence of Cy1, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and wherein R^{1′} is hydrogen; or wherein each of R¹ and R^{1′} together comprise ═O or ═NR³⁶; wherein each occurrence of R³⁶, when present, is independently selected from hydrogen and C1-C12 alkyl; wherein each of R² and R³ is independently selected from hydrogen, —OH, C1-C12 hydroxy, and halogen; or wherein each of R₂ and R₃ together comprise —O—; wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; wherein each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, (C1-C30 alkyl)OC(O)NR^{35a}R^{35b}, Cy¹, Ar¹, —(C1-C30 alkyl)Ar¹, —OAr¹, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)R⁴⁰, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰; wherein each occurrence of R⁴⁰, when present, is independently a C1-C30 alkyl functionalized with a group selected from —SH, —NH₂, —NHOH, terminal alkyne, haloacetyl, maleimide, isothiocyanate, N-hydroxysuccinimde, succinimidyl ester, tetrafluorophenyl ester, sulfodichlorophenol ester, and hydrazide; wherein each occurrence of R⁴¹ and R⁴², when present, is independently selected from hydrogen and C1-C12 alkyl; wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴; wherein each occurrence of Ar⁴, when present, is a structure represented by a formula:

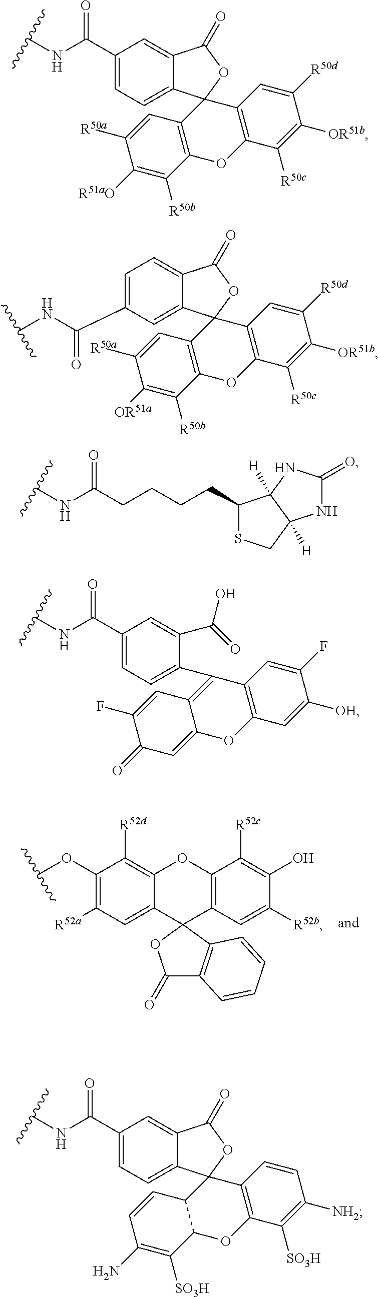

wherein each of R^{50a}, R^{50b}, R^{50c}, and R^{50d}, when present, is independently selected from hydrogen, —F, and —Cl; wherein each of R^{51a} and R^{51b}, when present, is independently selected from hydrogen and —C(O)(C1-C12 alkyl); wherein each of R^{52a}, R^{52b}, R^{52c}, and R^{52d}, when present, is independently selected from hydrogen, —F, and —Cl; wherein each occurrence of Ar³, when present, is a structure represented by a formula selected from:

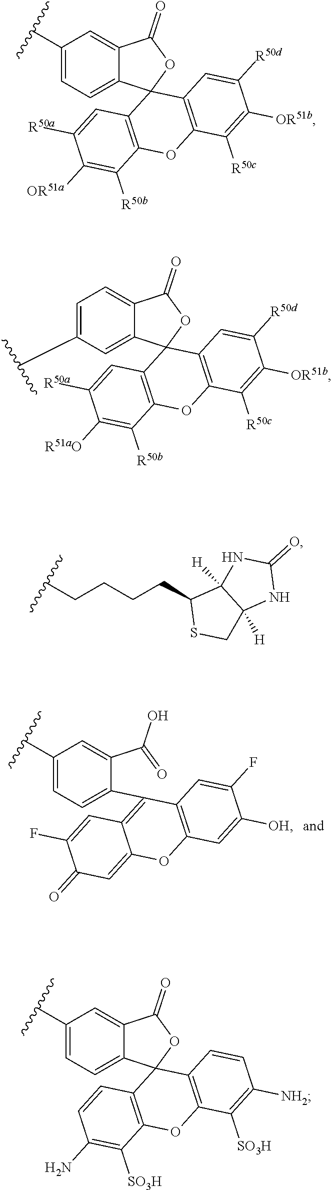

or wherein one of R⁶ and R^{6′} is absent; wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl); or wherein each of R⁷ and R^{7′} together comprise ═O; or wherein one of R⁷ and R^{7′} is absent; wherein each of R¹¹ and R¹² is independently selected from hydrogen, —OH, C1-C8 hydroxy, C1-C6 alkyl, C1-C8 alkoxy, and —OC(O)(C1-C8 alkyl); wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide); wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R²¹ is selected from hydrogen and C1-C6 alkyl; wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide); wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each of R²⁶ and R^{26′} is independently selected from hydrogen, —OH, C1-C8 hydroxy, and C1-C8 alkoxy; or wherein each of R²⁶ and R^{26′} together comprise ═O; wherein R²⁷ is selected from hydrogen and C1-C6 alkyl; wherein each of R²⁸ and R²⁹ is independently selected from hydrogen and halogen; or wherein each of R²⁸ and R²⁹ together comprise —O— or —N(R³⁷)—; wherein R³⁷, when present, is selected from hydrogen, CT-C4 alkyl, —SO₂R⁷¹, and a structure having a formula:

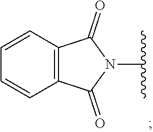 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that at least one of R⁶ and R^{6′} is —OC(O)R⁴⁰ or —NR⁴¹C(O)R⁴⁰, and provided that when one or both of R²⁸ and R²⁹ is hydrogen then the occurrence of - - - - - - at C-22/C-23 is a double covalent bond, or a pharmaceutically acceptable salt thereof, and a pharmaceutically acceptable carrier.

In one aspect, disclosed are pharmaceutical compositions comprising a therapeutically effective amount of a compound having a structure represented by a formula:

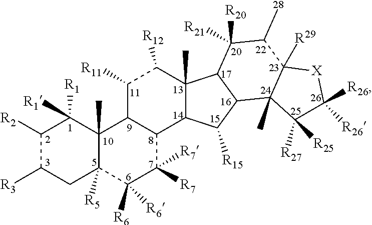

wherein each occurrence of - - - - - - is a single or double covalent bond; wherein X is selected from O, NR^{x} and C(R^{x})₂; wherein each occurrence of R^{x}, when present, is independently selected from hydrogen and C1-C6 alkyl; wherein R^{x} is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar1, and —OAr¹; wherein each occurrence of R³¹, R³², R³⁴, R^{35a}, and R^{35b}, when present, is independently selected from hydrogen and C1-C12 alkyl; wherein each occurrence of R³³, when present, is independently selected from hydrogen, C1-C12 alkyl, and monocyclic aryl monosubstituted with a methyl group; wherein each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and wherein R^{1′} is hydrogen; or wherein each of R¹ and R^{1′} together comprise ═O or ═NR³⁶; wherein each occurrence of R³⁶, when present, is independently selected from hydrogen and C1-C12 alkyl; wherein each of R² and R³ is independently selected from hydrogen, —OH, C1-C12 hydroxy, and halogen; or wherein each of R₂ and R₃ together comprise —O—; wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl); or wherein each of R⁷ and R^{7′} together comprise ═O; or wherein one of R⁷ and R^{7′} is absent; wherein each of R⁸ and R^{8′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy1, Ar¹, —(C1-C30 alkyl)Ar¹, —OAr¹, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, —NR⁴¹C(O)(C1-C30 alkyl)-L-Z, —NR⁴¹C(O)(C1-C30 alkyl)-L-(C1-C30 alkyl)-Z, and —NR⁴¹C(O)-L-(C1-C30 alkyl)-Z; wherein L is a linker; wherein Z is selected from an antibody, an antibody fragment, a vitamin, a hormone, a carbohydrate, a molecular ligand, an aptamer, a non-antibody protein, a peptide, a nucleic acid, a fluorophore, and a drug; wherein each occurrence of R⁴¹ and R⁴², when present, is independently selected from hydrogen and C1-C12 alkyl; wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴; wherein each occurrence of Ar⁴, when present, is a structure represented by a formula:

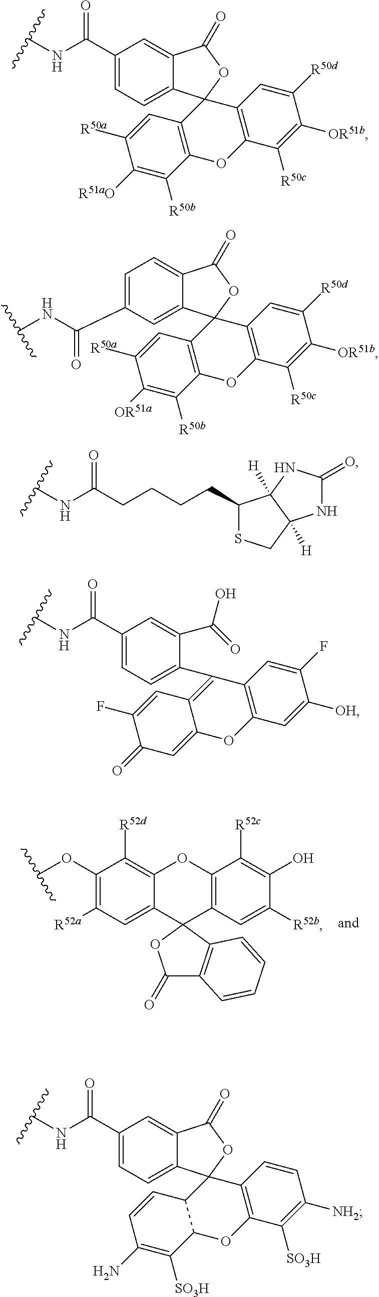

wherein each of R^{50a}, R^{50b}, R^{50c}, and R^{50d}, when present, is independently selected from hydrogen, —F, and —Cl; wherein each of R^{51a} and R^{51b}, when present, is independently selected from hydrogen and —C(O)(C1-C12 alkyl); wherein each of R^{52a}, R^{52b}, R^{52c}, and R^{52d}, when present, is independently selected from hydrogen, —F, and —Cl; wherein each occurrence of Ar³, when present, is a structure represented by a formula selected from:

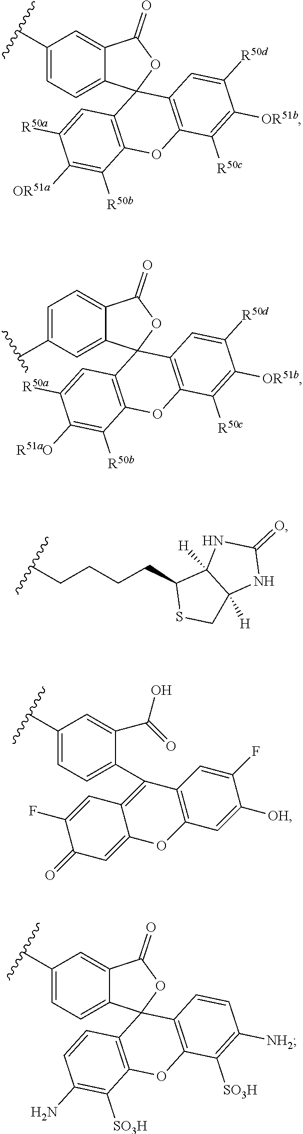

or wherein one of R⁸ and R^{8′} is absent; wherein each of R¹¹ and R¹² is independently selected from hydrogen, —OH, C1-C8 hydroxy, C1-C6 alkyl, C1-C8 alkoxy, and —OC(O)(C1-C8 alkyl); wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide); wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R²¹ is selected from hydrogen and C1-C6 alkyl; wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide); wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each of R²⁶ and R^{26′} is independently selected from hydrogen, —OH, C1-C8 hydroxy, and C1-C8 alkoxy; or wherein each of R²⁶ and R^{26′} together comprise ═O; wherein R²⁷ is selected from hydrogen and C1-C6 alkyl; wherein each of R²⁸ and R²⁹ is independently selected from hydrogen and halogen; or wherein each of R²⁸ and R²⁹ together comprise —O— or —N(R³⁷)—; wherein R³⁷, when present, is selected from hydrogen, C1-C4 alkyl, —SO₂R⁷¹, and a structure having a formula:

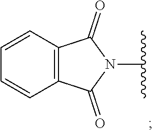 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that one and only one of R⁸ and R^{8′} is —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl)-L-Z, —NR⁴¹C(O)(C1-C30 alkyl)-L-(C1-C30 alkyl)-Z, or —NR⁴¹C(O)-L-(C1-C30 alkyl)-Z, and provided that when one or both of R²⁸ and R²⁹ is hydrogen then the occurrence of - - - - - - at C-22/C-23 is a double covalent bond, or a pharmaceutically acceptable salt thereof, and a pharmaceutically acceptable carrier.

In one aspect, disclosed are pharmaceutical compositions comprising a therapeutically effective amount of a compound having a structure selected from:

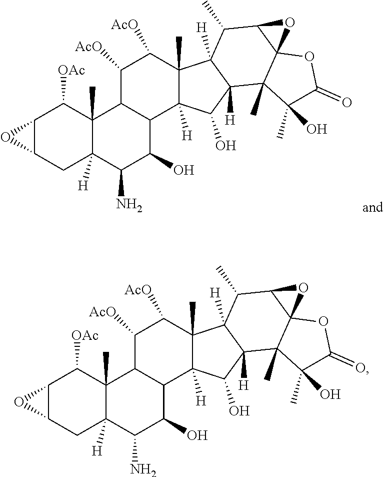 [SMILES: CC(=O)O[C@H]1C2C([C@@H](O)[C@@H](N)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@H](N)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

or a pharmaceutically acceptable salt thereof, and a pharmaceutically acceptable carrier.

In one aspect, disclosed are pharmaceutical compositions comprising a therapeutically effective amount of a compound having a structure selected from:

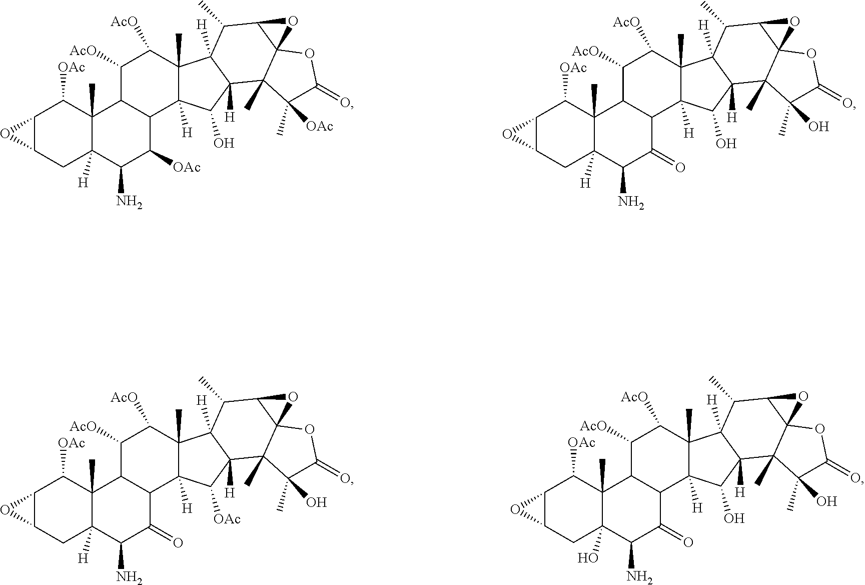

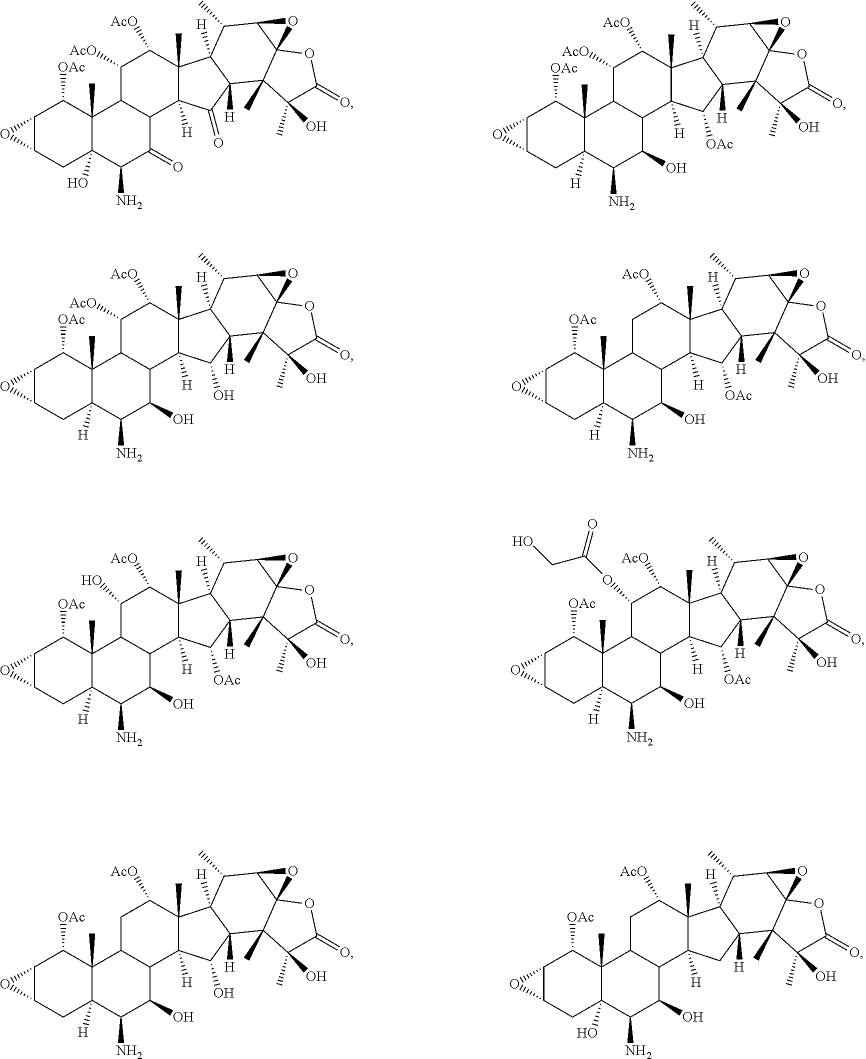

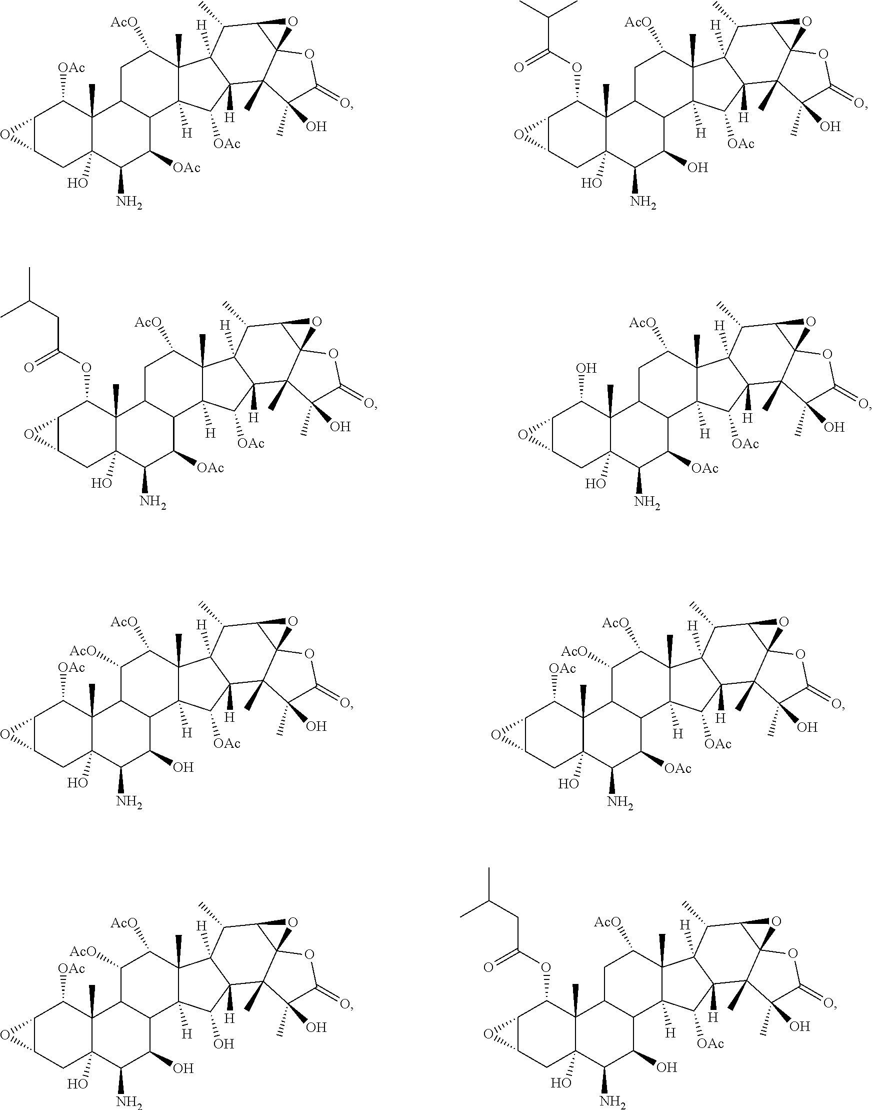

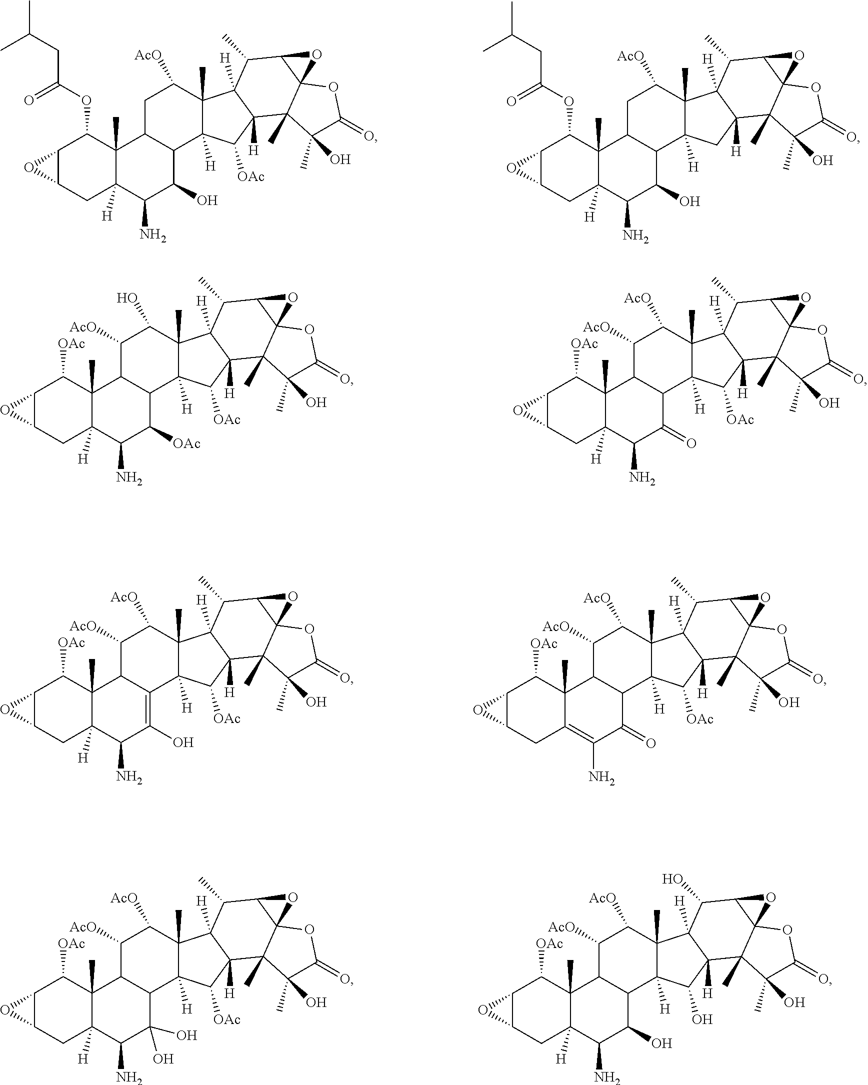

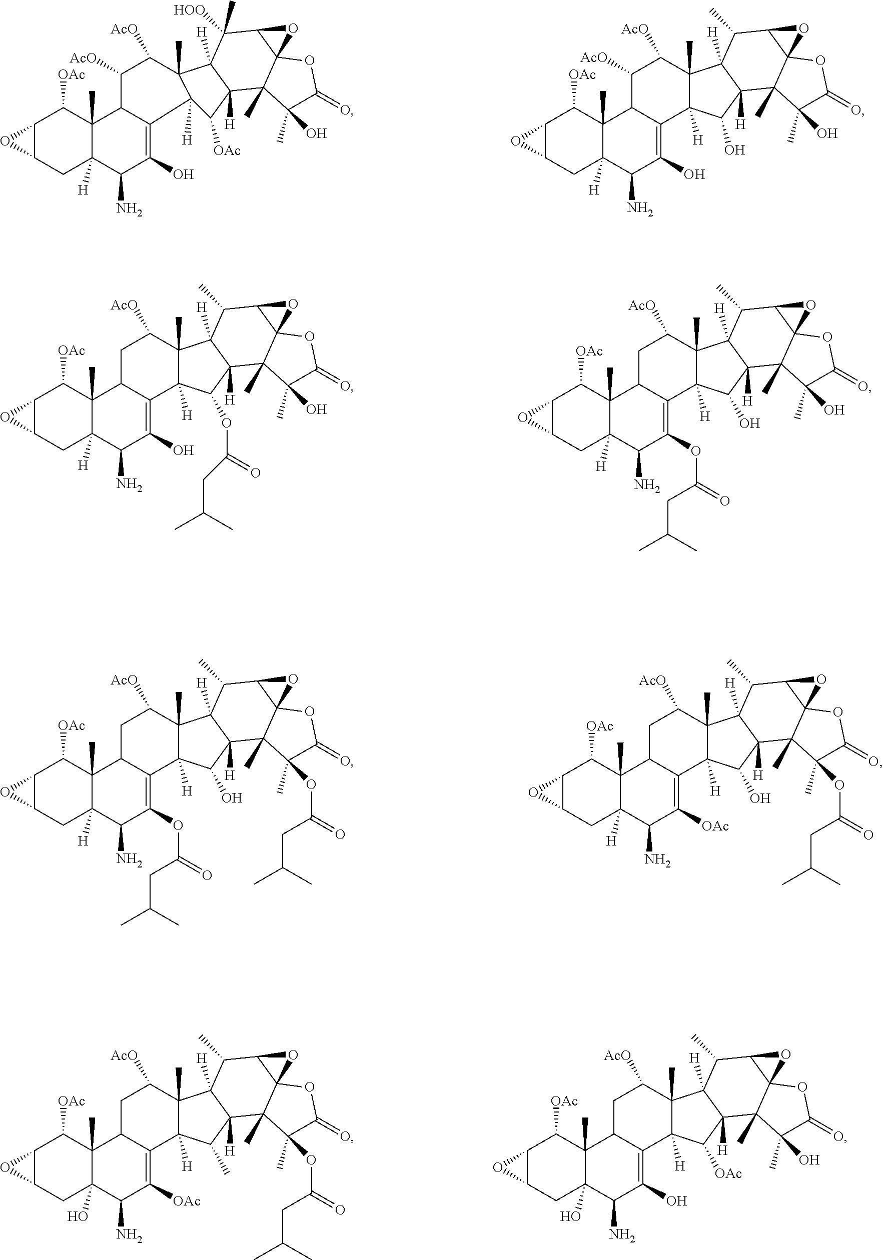

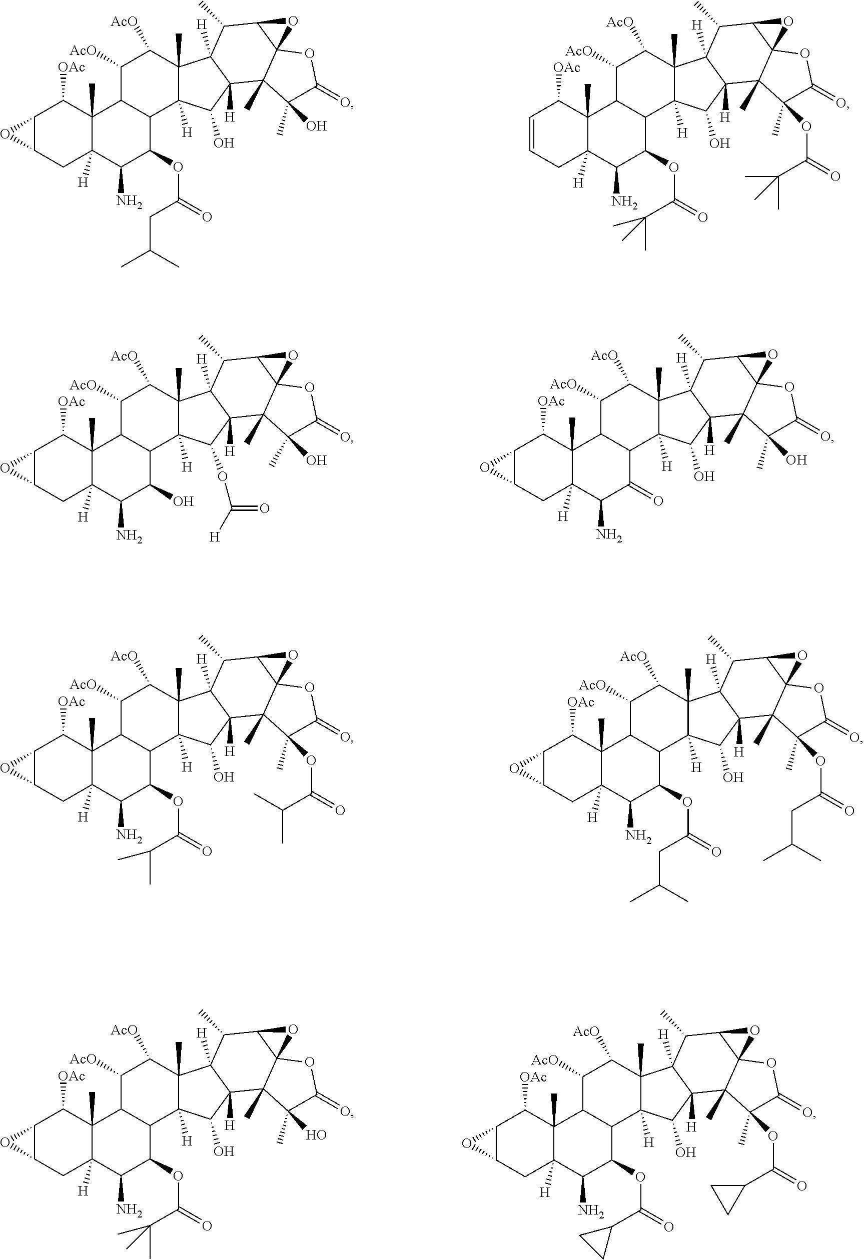

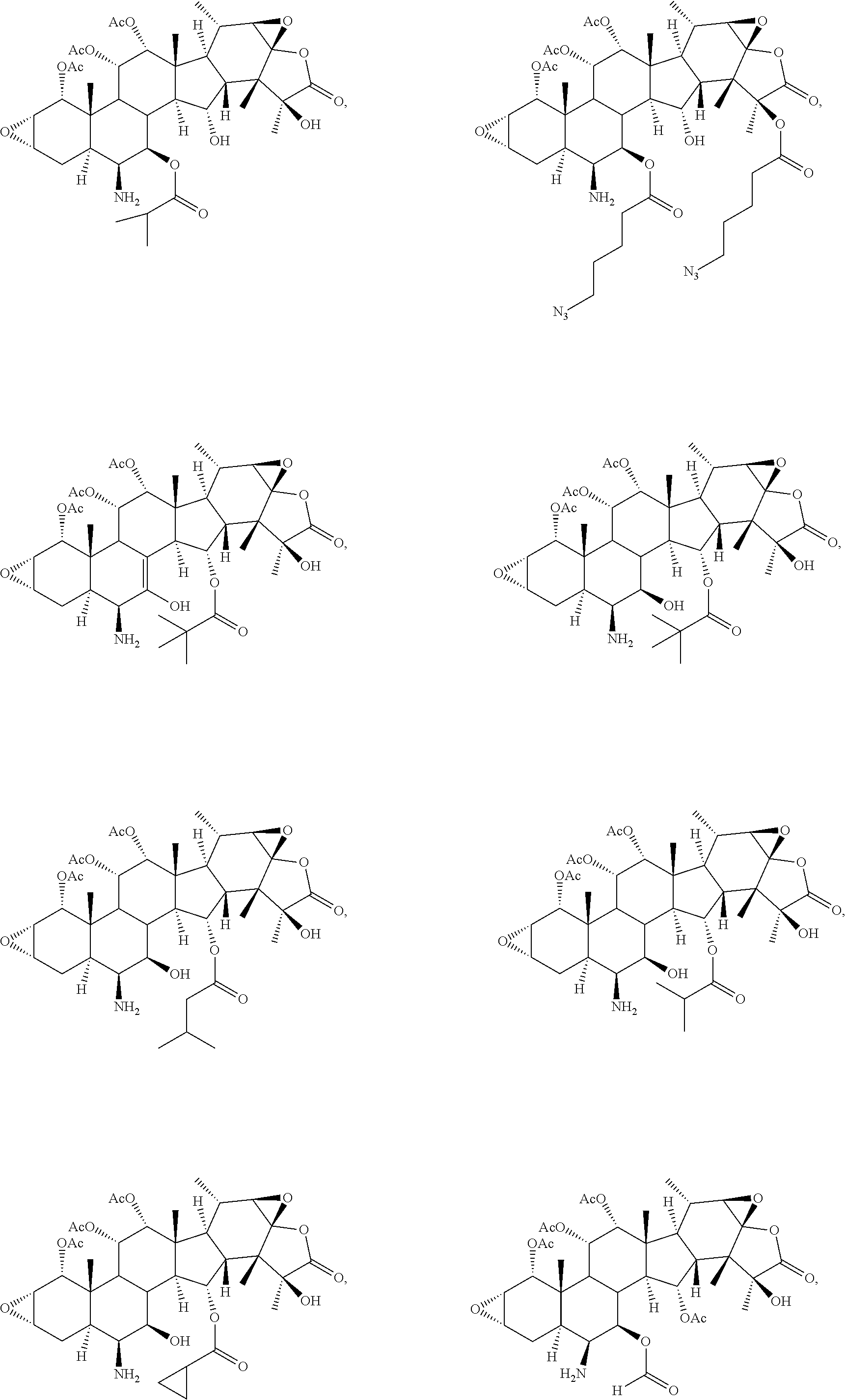

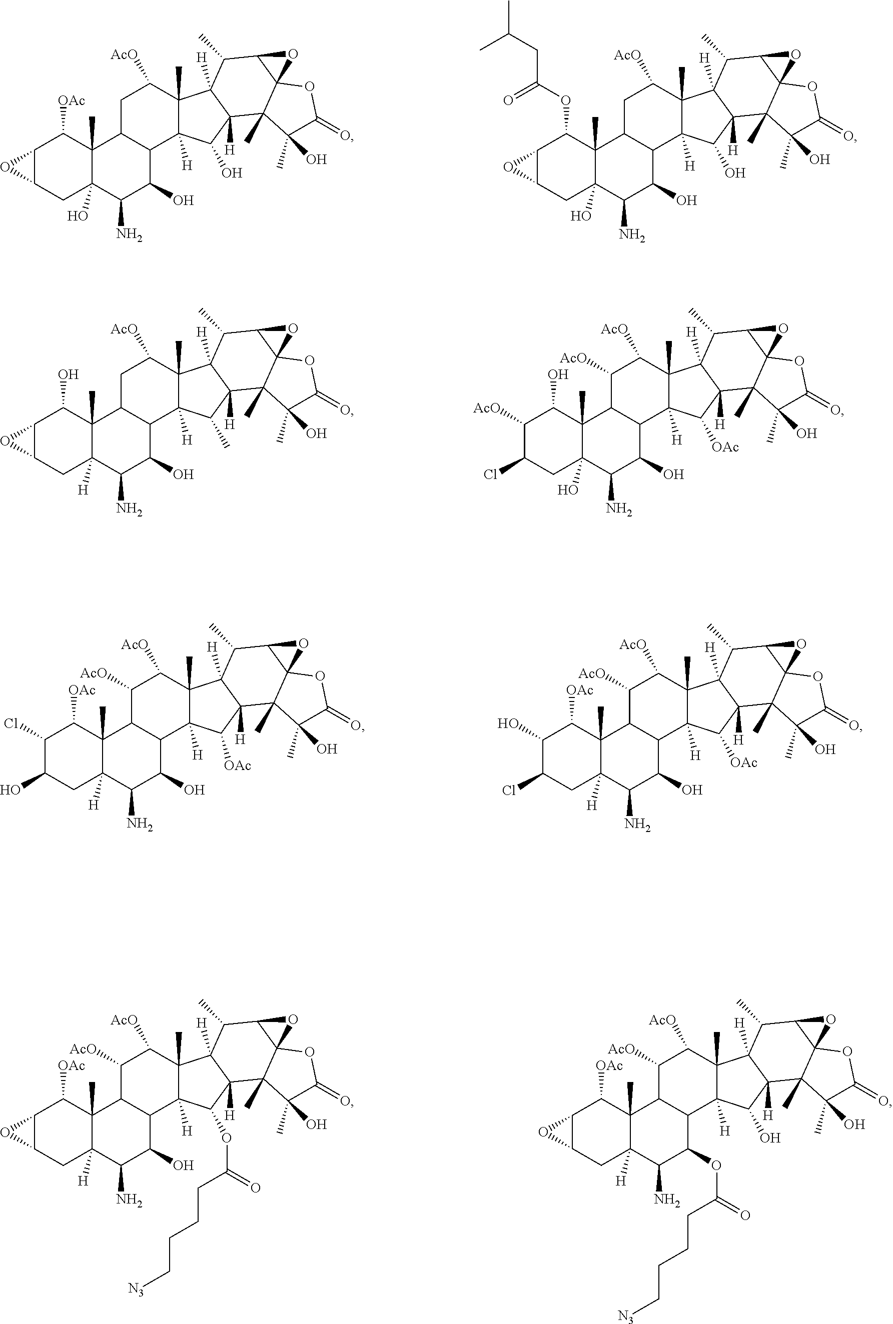

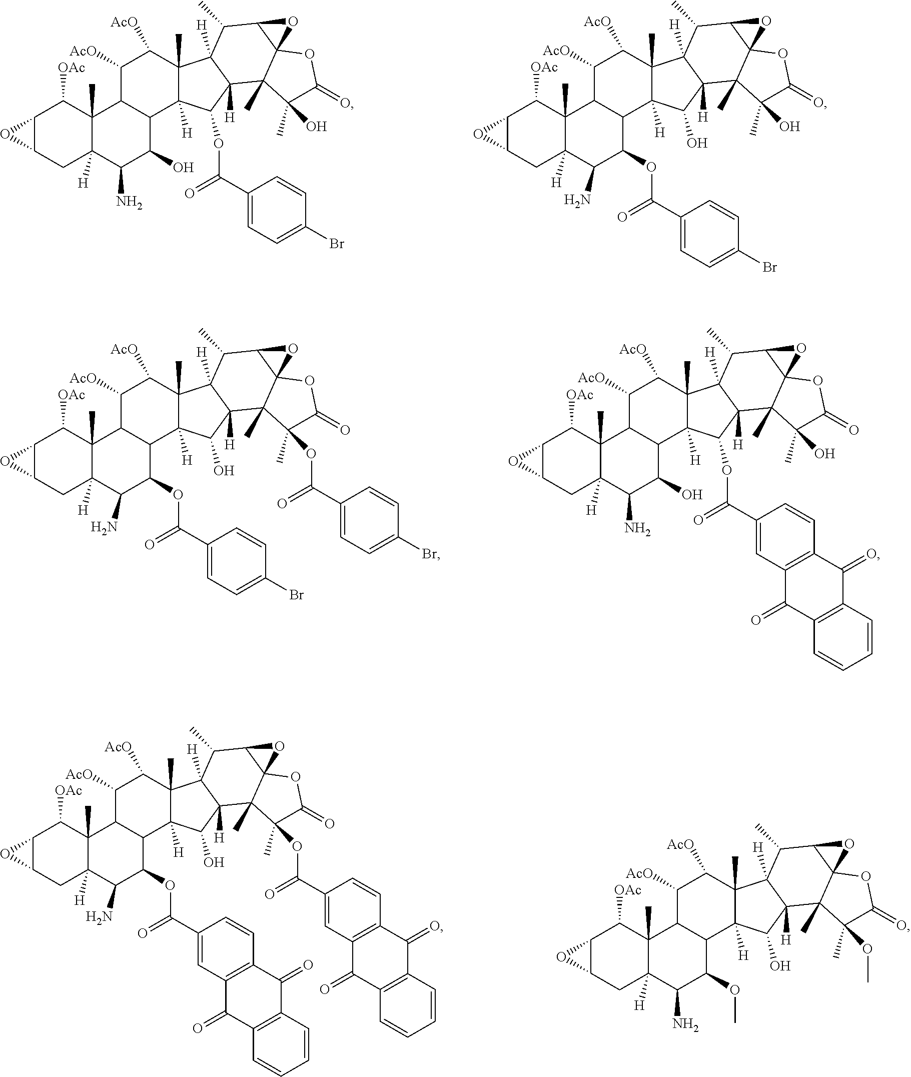

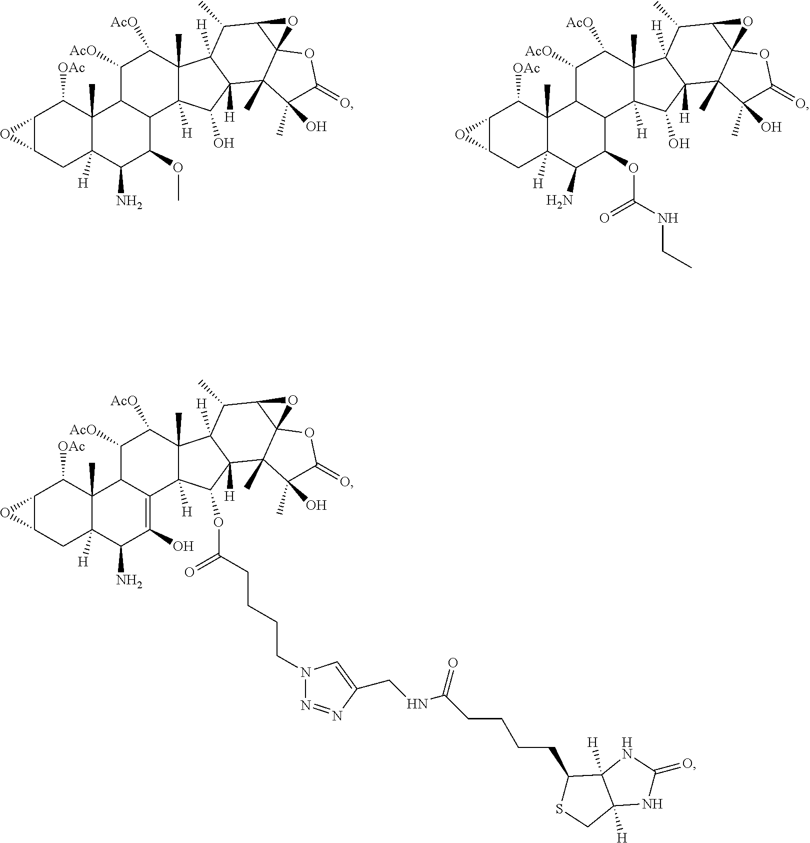

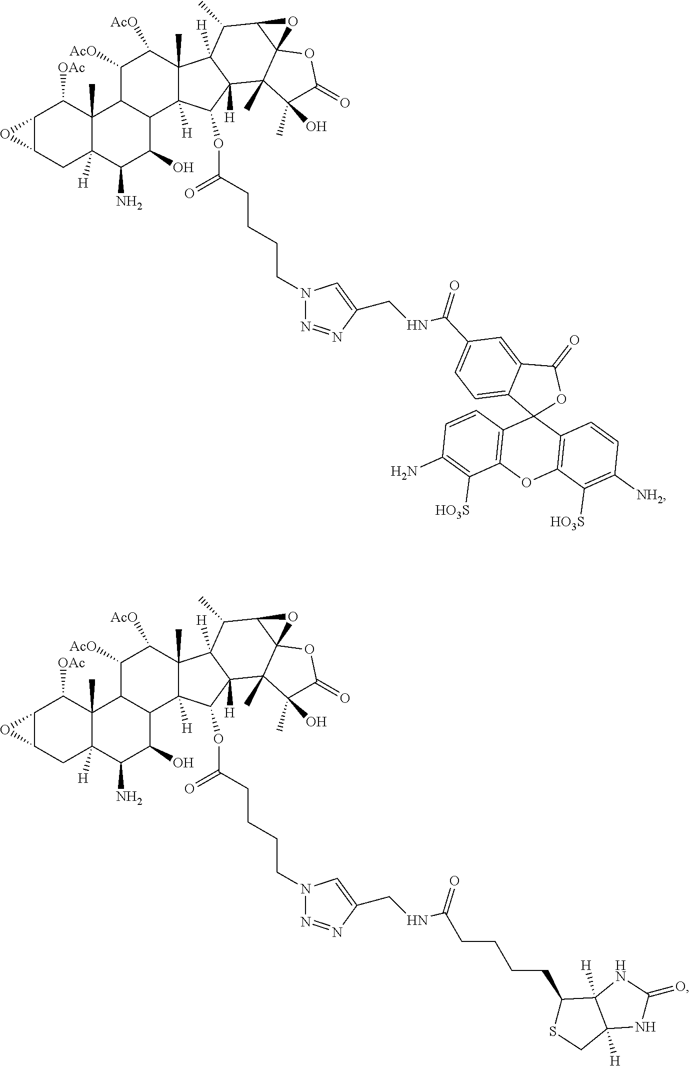

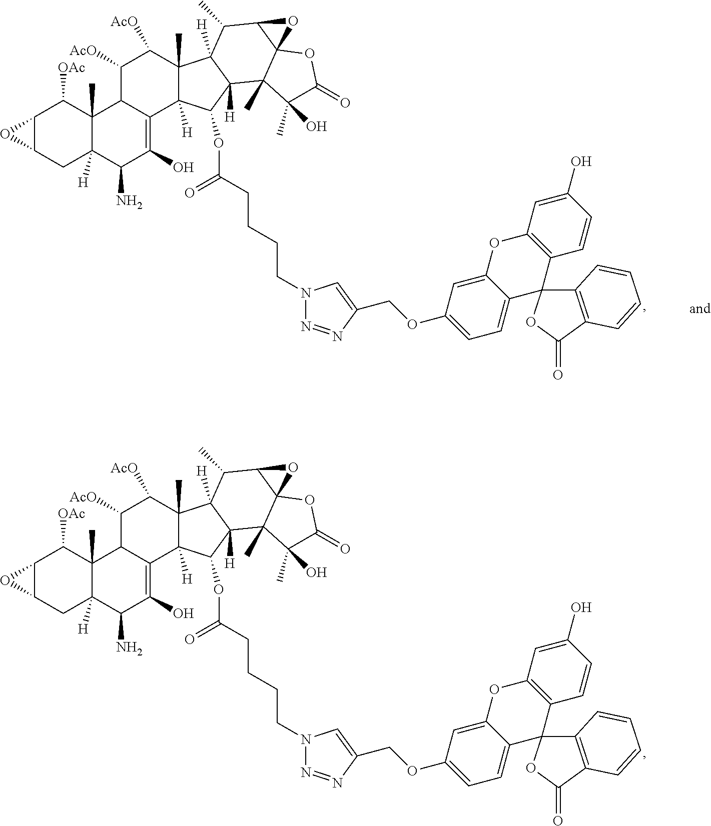

or a pharmaceutically acceptable salt thereof, and a pharmaceutically acceptable carrier.

In one aspect, disclosed are pharmaceutical compositions comprising a therapeutically effective amount of a compound selected from:

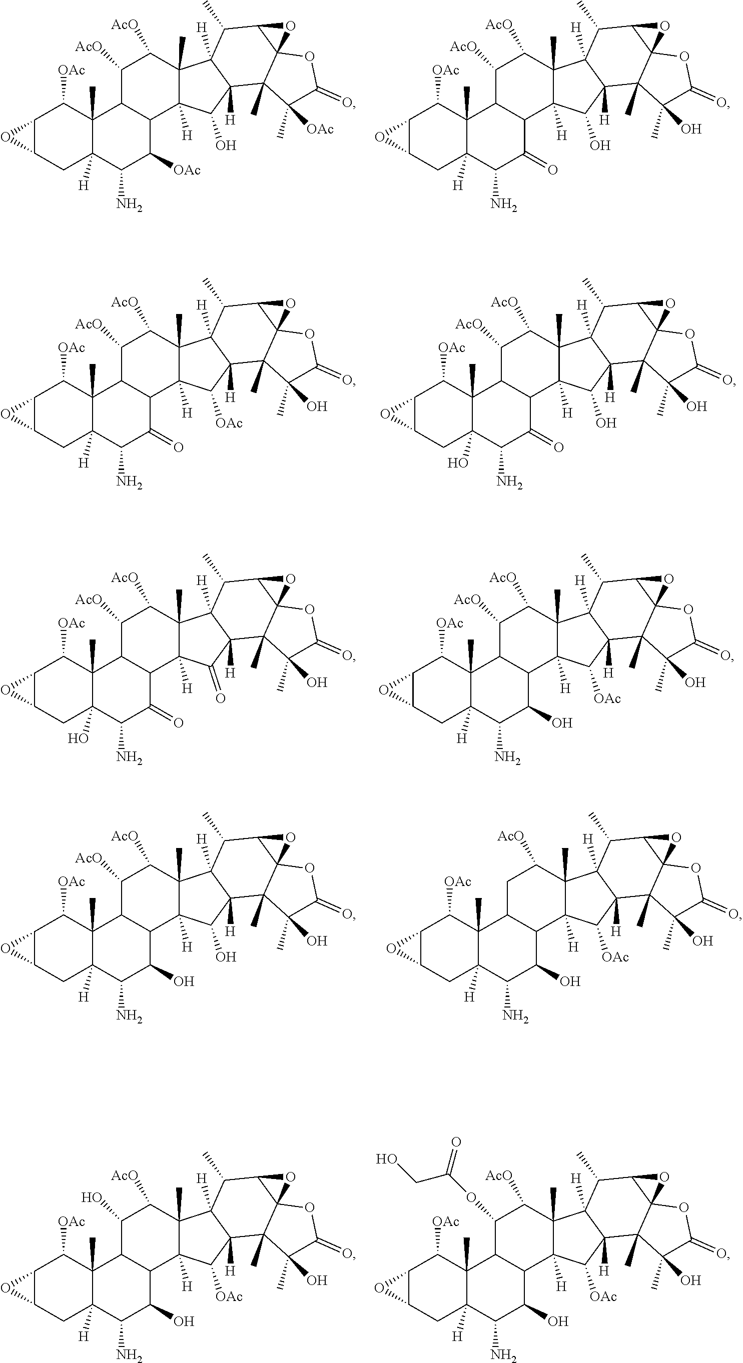

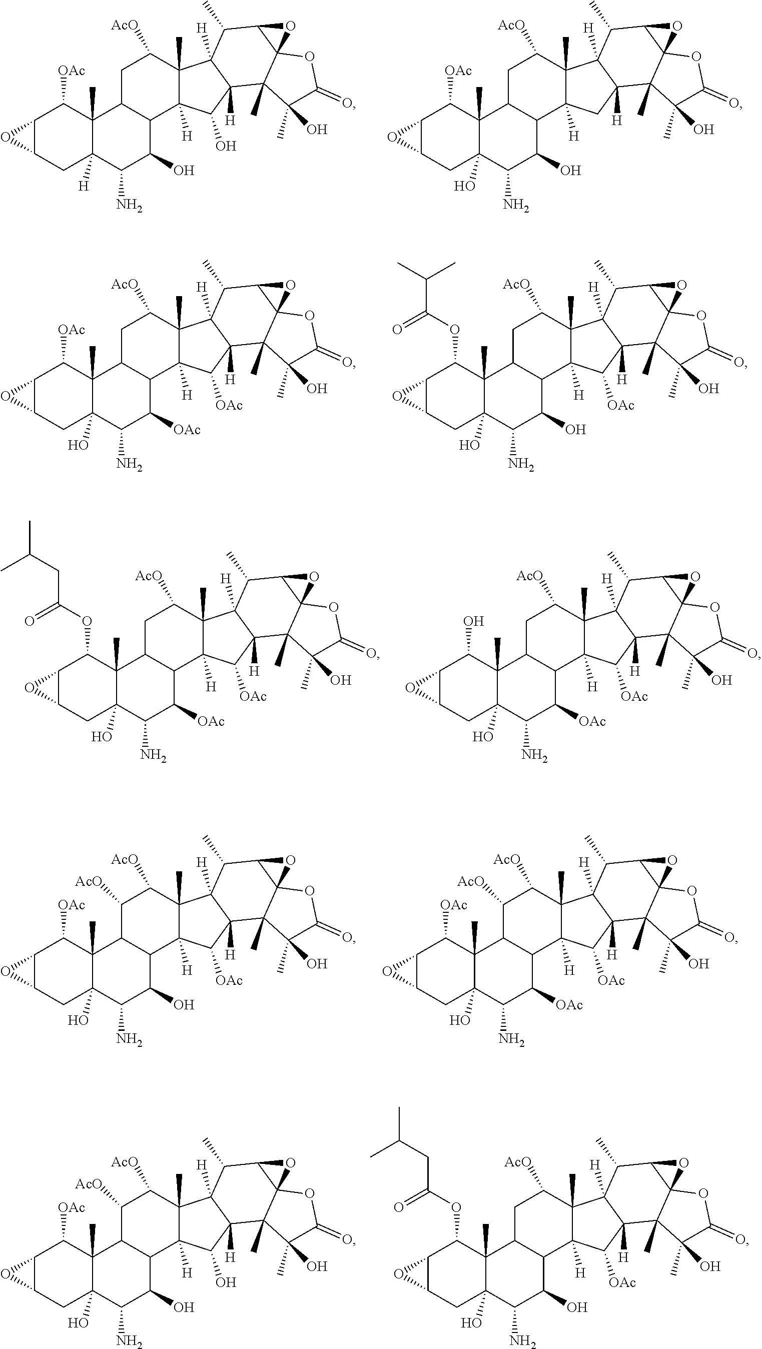

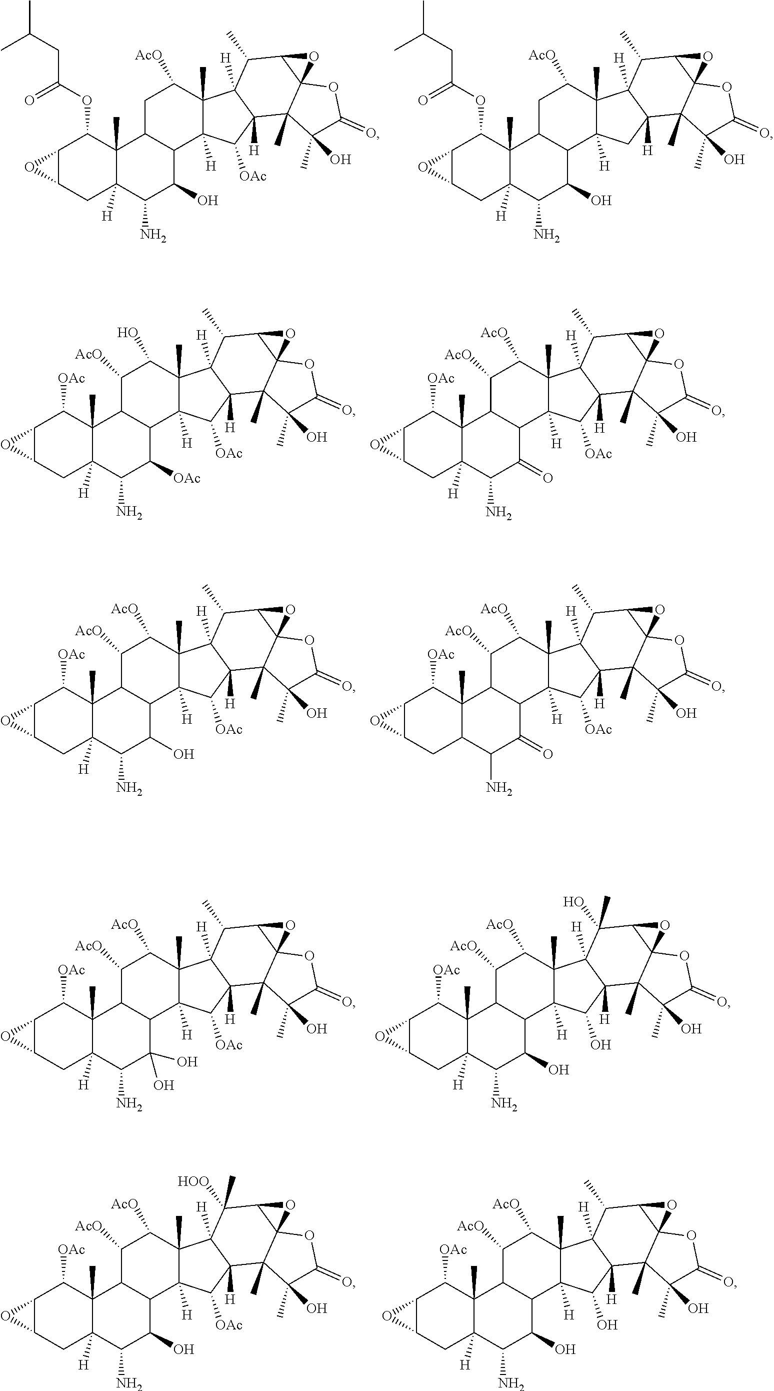

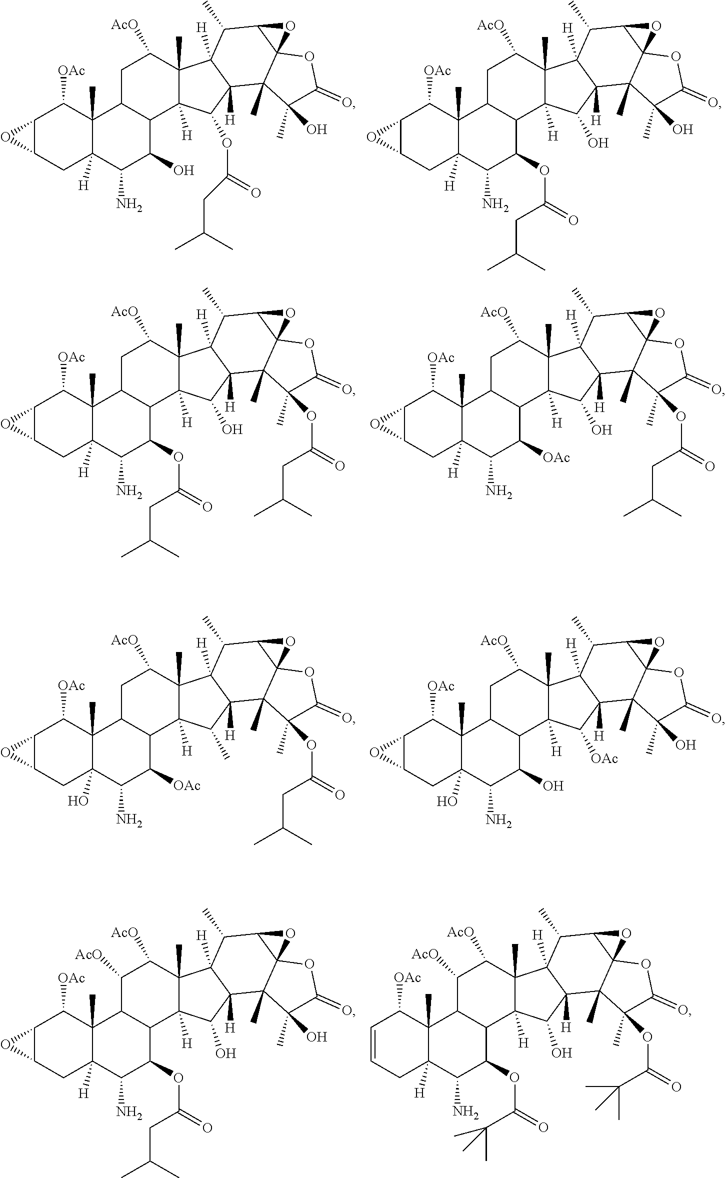

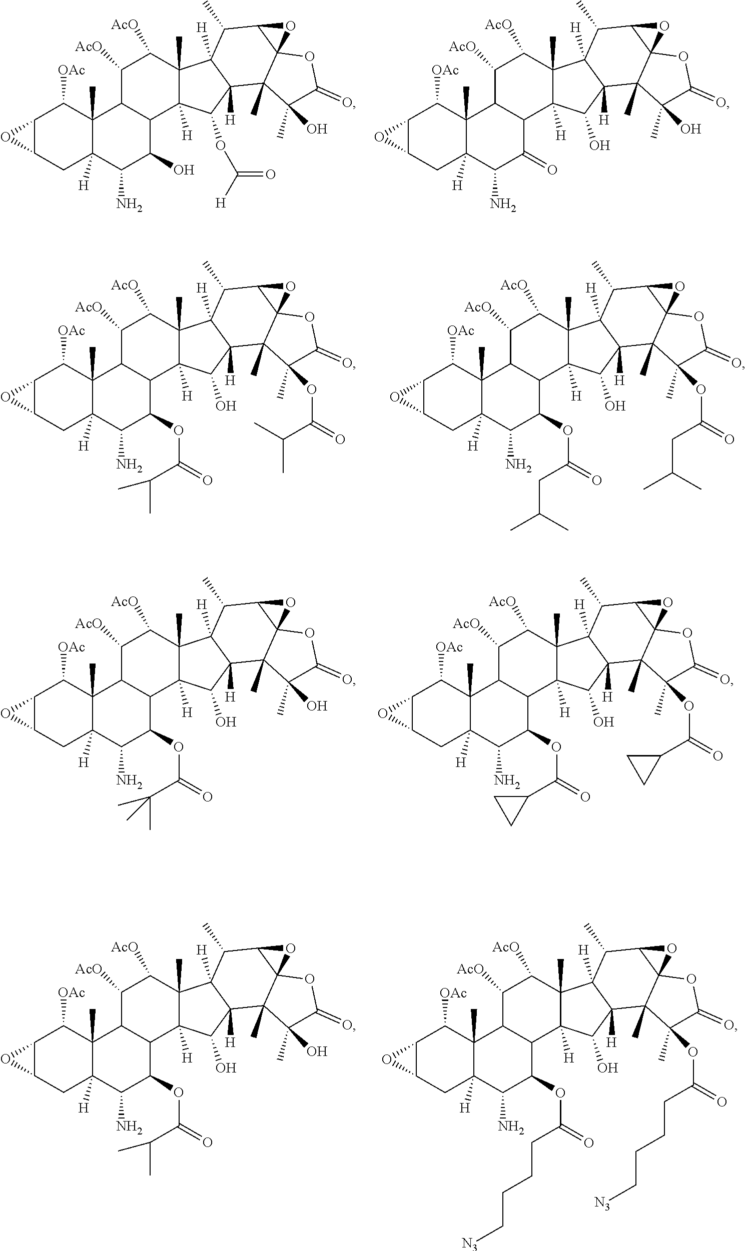

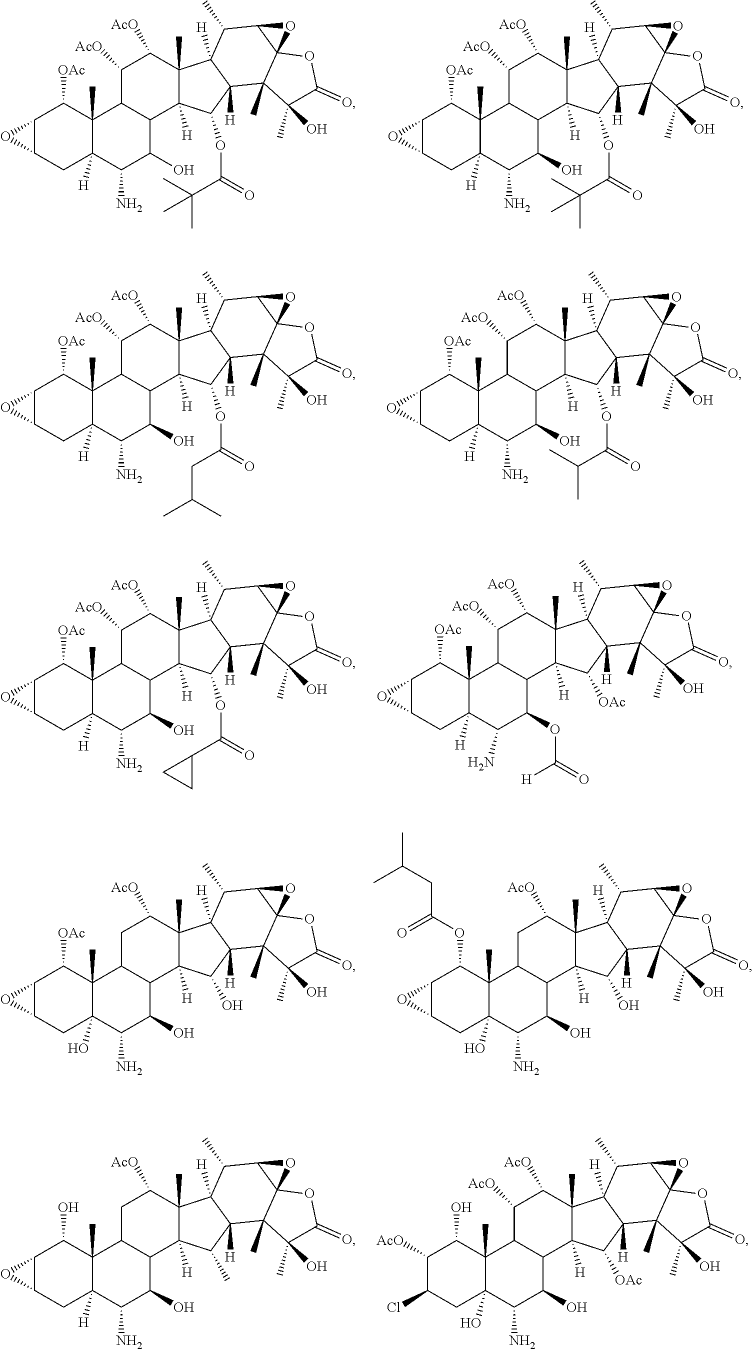

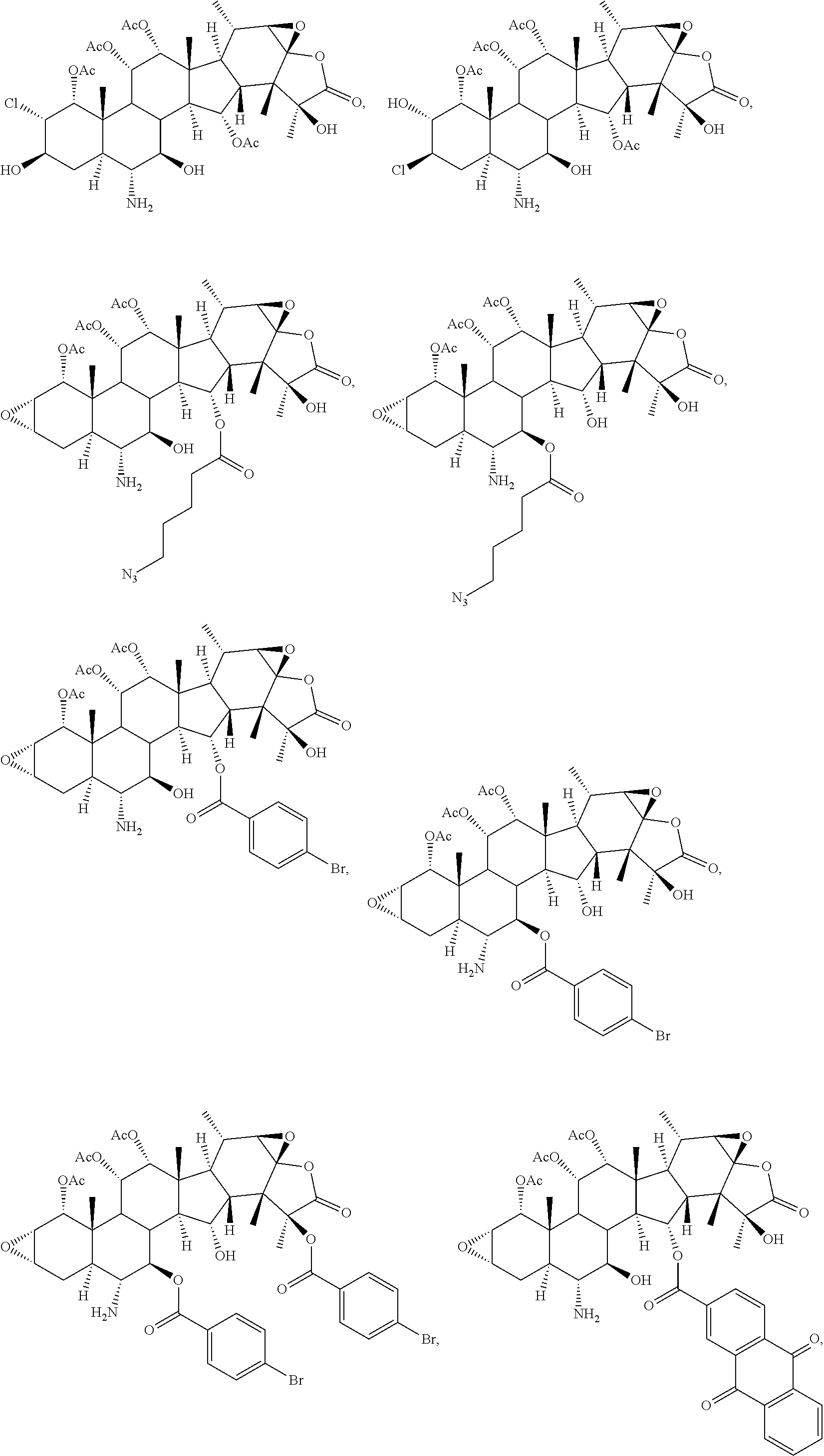

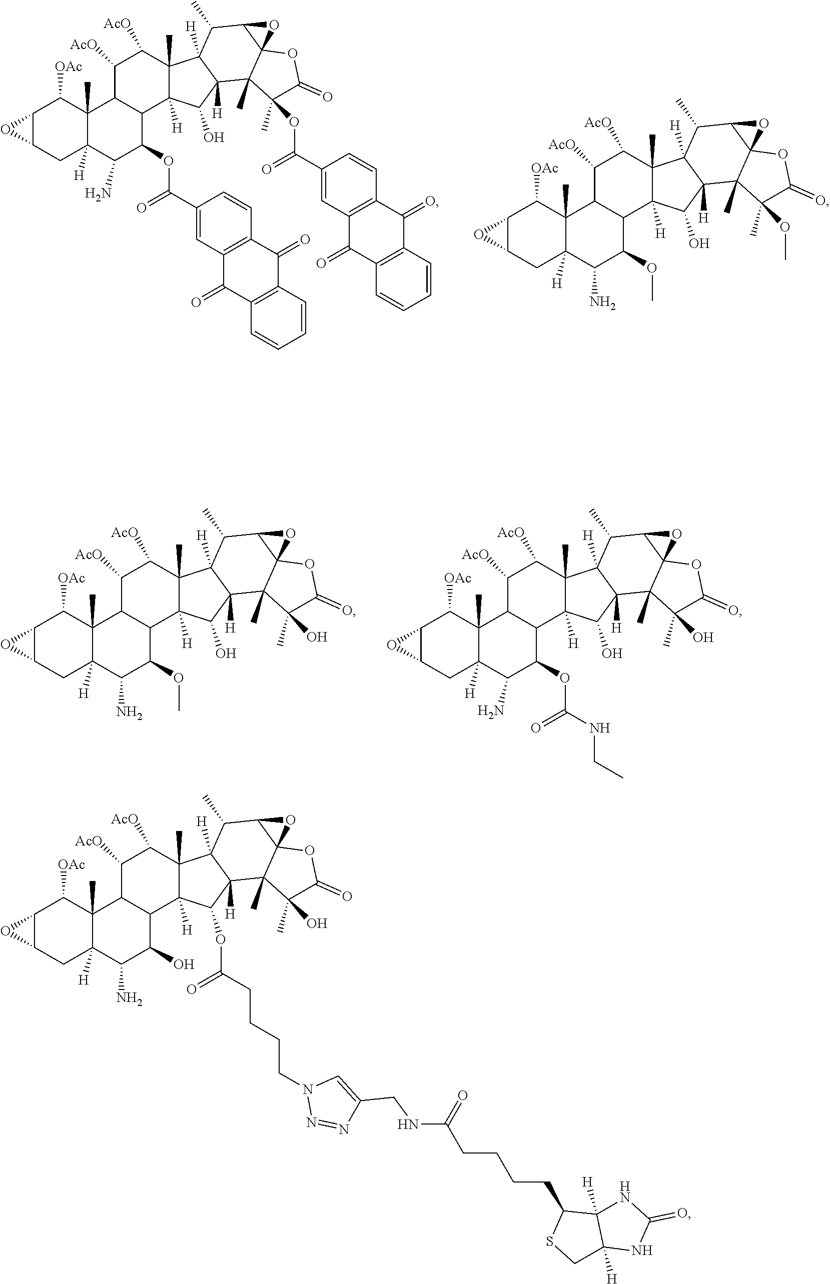

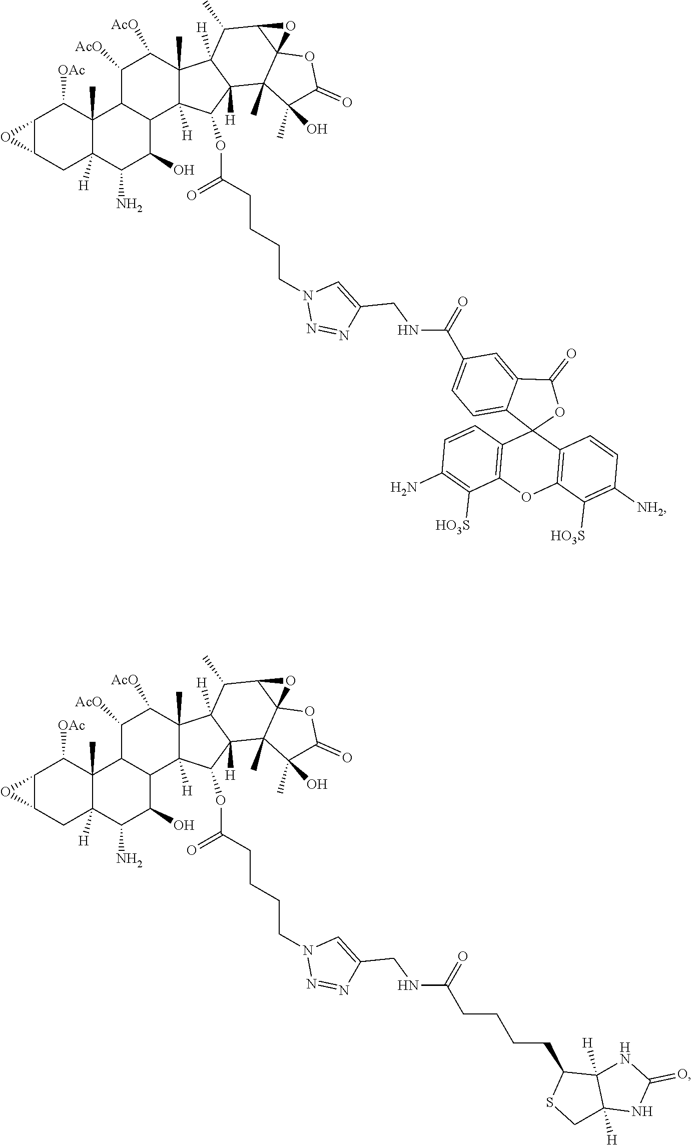

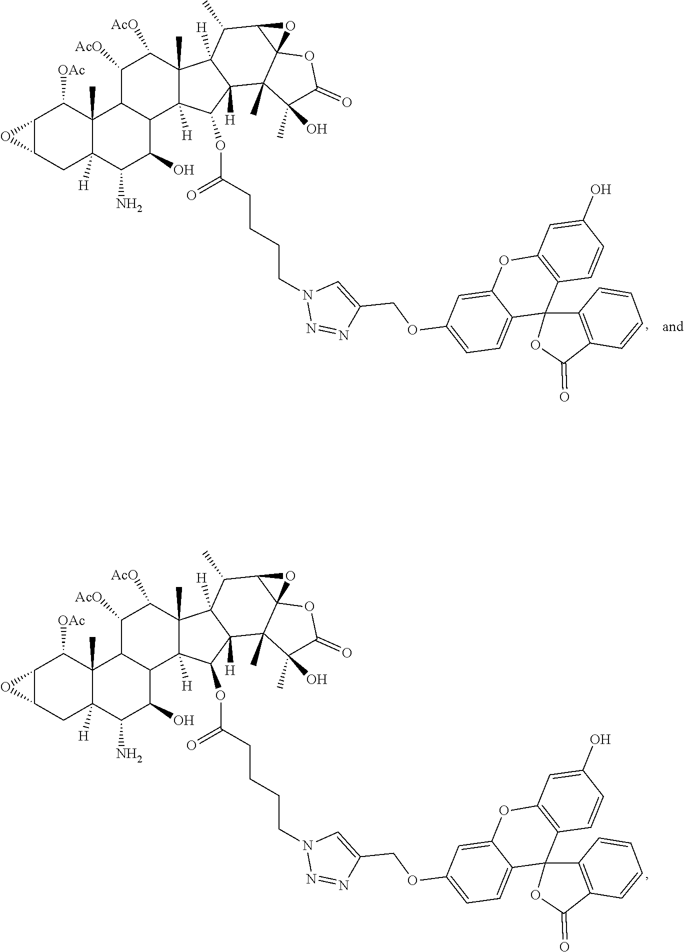 [SMILES: CC(=O)O[C@H]1C2C([C@@H](O)[C@H](N)[C@H]3C[C@@H]4O[C@@H]4[C@H](OC(C)=O)[C@]23C)[C@@H]2[C@@H](OC(=O)CCCCn3cc(COc4ccc5c(c4)Oc4cc(O)ccc4C54OC(=O)c5ccccc54)nn3)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@H](N)[C@H]3C[C@@H]4O[C@@H]4[C@H](OC(C)=O)[C@]23C)[C@@H]2[C@H](OC(=O)CCCCn3cc(COc4ccc5c(c4)Oc4cc(O)ccc4C54OC(=O)c5ccccc54)nn3)[C@@H]3[C@H]([C@H](C)[C@H]4O[C@]45OC(=O)[C@@](C)(O)[C@]35C)[C@@]2(C)[C@H]1OC(C)=O]

or a pharmaceutically acceptable salt thereof, and a pharmaceutically acceptable carrier.

Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, such as for example, tetramethylammonium hydroxide. Chemical modification of a pharmaceutical compound into a salt is a known technique to obtain improved physical and chemical stability, hygroscopicity, flowability and solubility of compounds. See, e.g., H. Ansel et. al., Pharmaceutical Dosage Forms and Drug Delivery Systems (6th Ed. 1995) at pp. 196 and 1456-1457.

The pharmaceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In a further aspect, the pharmaceutical composition is administered to a mammal. In a still further aspect, the mammal is a human. In an even further aspect, the human is a patient.

In a further aspect, the pharmaceutical composition is administered following identification of the mammal in need of treatment of cancer. In a still further aspect, the mammal has been diagnosed with a need for treatment of cancer prior to the administering step.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

The choice of carrier will be determined in part by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, intraperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granule; (c) powders; (d) suspensions in an appropriate liquid; and (e) suitable emulsions. Liquid formulations may include diluents, such as water, cyclodextrin, dimethyl sulfoxide and alcohols, for example, ethanol, benzyl alcohol, propylene glycol, glycerin, and the polyethylene alcohols including polyethylene glycol, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, the addition to the active ingredient in an inert base, such as gelatin and glycerin, or sucrose and acadia, emulsions, and gels containing, in addition to the active ingredient, such carriers as are known in the art.

The compounds of the present disclosure alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1, 3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldiallylammonium halides, and alkylpyridinium halides, (b) anionic detergents such as, for example, alkyl, aryl, and olefin sulfonates, alkyl olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4^{th} ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

One skilled in the art will appreciate that suitable methods of exogenously administering a compound of the present disclosure to an animal are available, and, although more than one route can be used to administer a particular compound, a particular route can provide a more immediate and more effective reaction than another route.

As regards these applications, the present method includes the administration to an animal, particularly a mammal, and more particularly a human, of a therapeutically effective amount of the compound effective in the treatment of a hyperproliferative disorder. The method also includes the administration of a therapeutically effect amount of the compound for the treatment of patient having a predisposition for being afflicted with a hyperproliferative disorder. The dose administered to an animal, particularly a human, in the context of the present invention should be sufficient to affect a therapeutic response in the animal over a reasonable time frame. One skilled in the art will recognize that dosage will depend upon a variety of factors including the condition of the animal, the body weight of the animal, as well as the severity and stage of the virus.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

In a further aspect, the composition further comprises at least one agent known to treat a hyperproliferative disorder. In a still further aspect, the composition further comprises at least one agent known to have a side effect of increasing the risk of a hyperproliferative disorder.

In a further aspect, the composition further comprises at least one agent known to treat cancer. In a still further aspect, the cancer is selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma). In a still further aspect, the cancer is selected from a leukemia, colorectal cancer, pancreatic cancer, ovarian cancer, non-small cell lung carcinoma, and breast cancer.

In a further aspect, the composition further comprises at least one agent known to have a side effect of increasing the risk of cancer.

In a further aspect, the composition further comprises at least one agent known to treat a cardiovascular disease. Examples of cardiovascular diseases include, but are not limited to, coronary heart disease, stroke, hypertensive heart disease, inflammatory heart disease, and rheumatic heart disease,

In a further aspect, the composition further comprises at least one agent known to have a side effect of increasing the risk of cardiovascular disease.

In a further aspect, the composition comprises at least 50 wt % of the compound, based on the total weight of the composition. In a still further aspect, wherein the composition comprises at least 60 wt % of the compound, based on the total weight of the composition. In yet a further aspect, wherein the composition comprises at least 70 wt % of the compound, based on the total weight of the composition. In an even further aspect, wherein the composition comprises at least 80 wt % of the compound, based on the total weight of the composition. In a still further aspect, wherein the composition comprises at least 90 wt % of the compound, based on the total weight of the composition. In yet a further aspect, wherein the composition comprises at least 95 wt % of the compound, based on the total weight of the composition. In an even further aspect, wherein the composition comprises at least 99 wt % of the compound, based on the total weight of the composition.

It is understood that the disclosed compositions can be prepared from the disclosed compounds. It is also understood that the disclosed compositions can be employed in the disclosed methods of using.

### D. Methods of Making the Compounds

In various aspects, the inventions relates to methods of making compounds useful as cellular probes (e.g., fluorescence, biotin) and as antibody-drug conjugates (ADCs). The disclosed compounds are also useful to treat hyperproliferative disorders such as, for example, cardiovascular diseases such as, for example, coronary heart disease, stroke, hypertensive heart disease, inflammatory heart disease, and rheumatic heart disease, and cancers such as, for example, a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma). Thus, in one aspect, disclosed are methods of making a disclosed compound.

Compounds according to the present disclosure can, for example, be prepared by the several methods outlined below. A practitioner skilled in the art will understand the appropriate use of protecting groups [see: Greene and Wuts, Protective Groups in Organic Synthesis] and the preparation of known compounds found in the literature using the standard methods of organic synthesis. There may come from time to time the need to rearrange the order of the recommended synthetic steps, however this will be apparent to the judgment of a chemist skilled in the art of organic synthesis. The following examples are provided so that the invention might be more fully understood, are illustrative only, and should not be construed as limiting.

In one aspect, the disclosed compounds comprise the products of the synthetic methods described herein. In a further aspect, the disclosed compounds comprise a compound produced by a synthetic method described herein. In a still further aspect, the invention comprises a pharmaceutical composition comprising a therapeutically effective amount of the product of the disclosed methods and a pharmaceutically acceptable carrier. In a still further aspect, the invention comprises a method for manufacturing a medicament comprising combining at least one compound of any of disclosed compounds or at least one product of the disclosed methods with a pharmaceutically acceptable carrier or diluent.

1. Route I

In one aspect, taccalonolide analogs can be prepared as shown below.

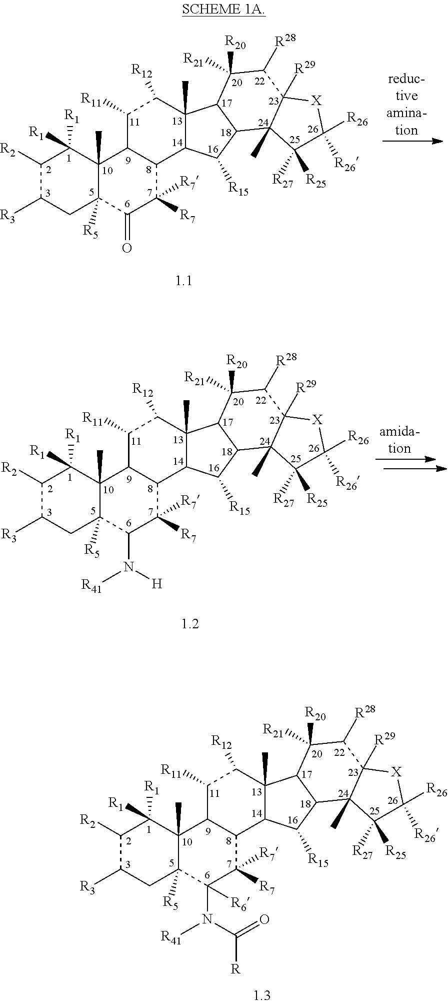

Compounds are represented in generic form, wherein R is C1-C30 alkyl, Ar², —(C1-C30 alkyl)Ar², Ar³, —(C1-C30 alkyl)OC(O)Ar³, —(C1-C30 alkyl)NR⁴²C(O)Ar³, or R⁴⁰ and with other substituents as noted in compound descriptions elsewhere herein. A more specific example is set forth below.

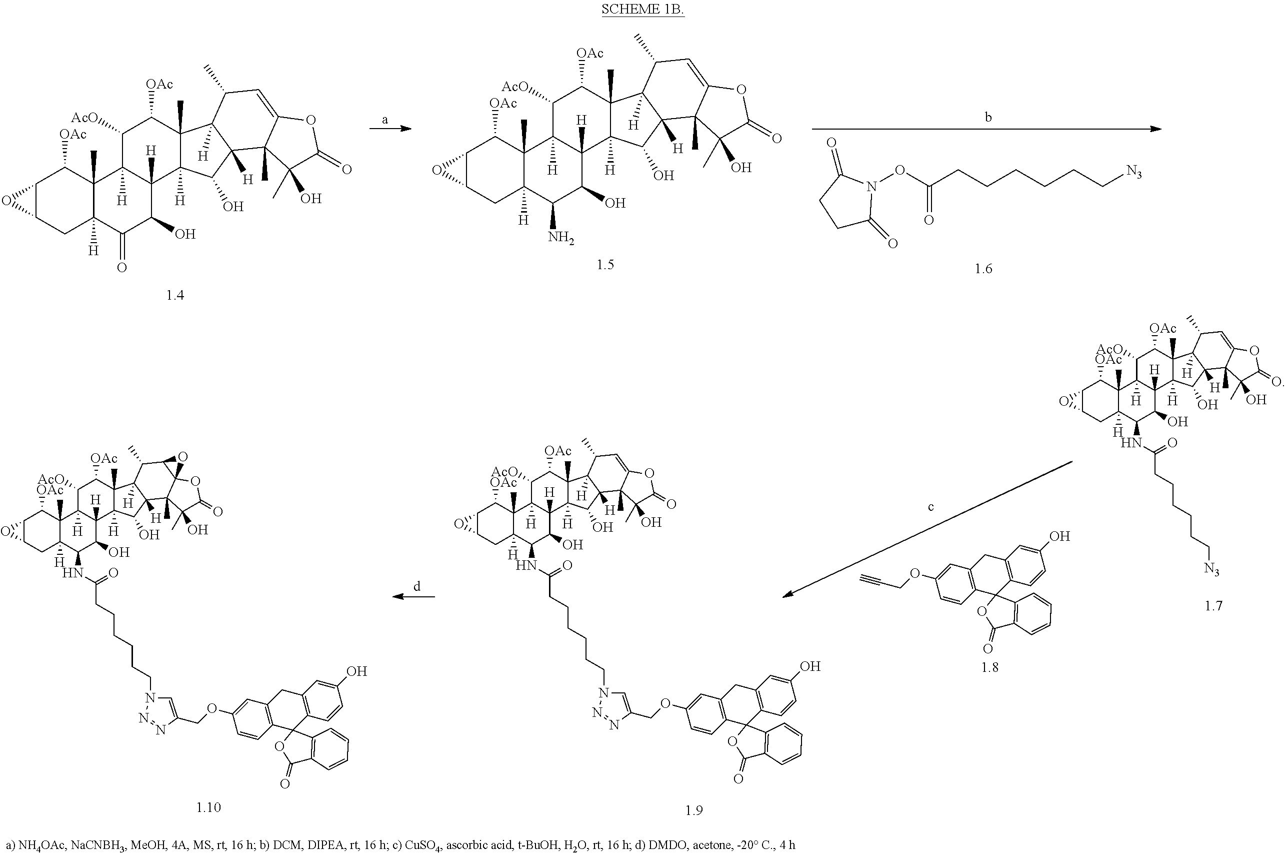

In one aspect, compounds of type 1.10, and similar compounds, can be prepared according to reaction Scheme 1B above. Thus, compounds of type 1.5 can be prepared by reductive amination of an appropriate ketone, e.g., 1.4 as shown above. Appropriate ketones are commercially available or prepared by methods known to one skilled in the art. The reductive amination is carried out in the presence of an appropriate reducing agent, e.g., sodium cyanoborohydride, and appropriate amine source, e.g., ammonium acetate, and an appropriate solvent, e.g., methanol, for an appropriate period of time, e.g., 16 hours. Compounds of type 1.7 can be prepared by amidation of an appropriate amine, e.g., 1.5 as shown above, and an appropriate succinimide, e.g., 1.6 as shown above. Appropriate succinimides are commercially available or prepared by methods known to one skilled in the art. The amidation is carried out in the presence of an appropriate base, e.g., N,N-diisopropylethylamine (DIPEA), in an appropriate solvent, e.g., dichloromethane (DCM), for an appropriate period of time, e.g., 16 hours. Compounds of type 1.9 can be prepared by cyclization of an appropriate azide, e.g., 1.7 as shown above, and an appropriate alkyne, e.g., 1.8 as shown above. Appropriate alkynes are commercially available or prepared by methods known to one skilled in the art. The cyclization is carried out in the presence of an appropriate catalyst, e.g., copper sulfate, and an appropriate acid, e.g., ascorbic acid, in an appropriate solvent system, e.g., t-butanol and water, for an appropriate period of time, e.g., 16 hours. Compounds of type 1.10 can be prepared by epoxidation of an appropriate alkene, e.g., 1.9 as shown above. The epoxidation is carried out in the presence of an appropriate oxidizing agent, e.g., dimethyldioxirane (DMDO) as shown above, in an appropriate solvent, e.g., acetone, at an appropriate temperature, e.g., −20° C., for an appropriate period of time, e.g., 4 hours. As can be appreciated by one skilled in the art, the above reaction provides an example of a generalized approach wherein compounds similar in structure to the specific reactants above (compounds similar to compounds of type 1.1 and 1.2), can be substituted in the reaction to provide substituted taccalonolide analogs similar to Formula 1.3.

2. Route II

In one aspect, taccalonolide analogs can be prepared as shown below.

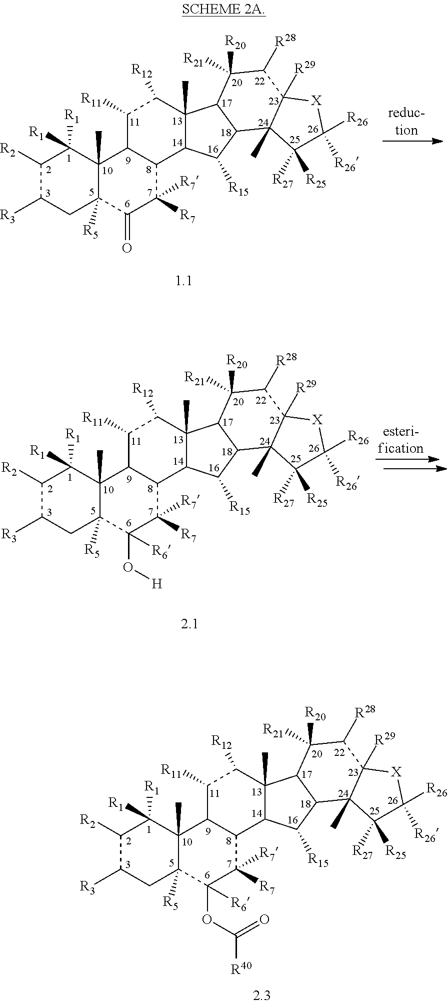

Compounds are represented in generic form, with substituents as noted in compound descriptions elsewhere herein. A more specific example is set forth below.

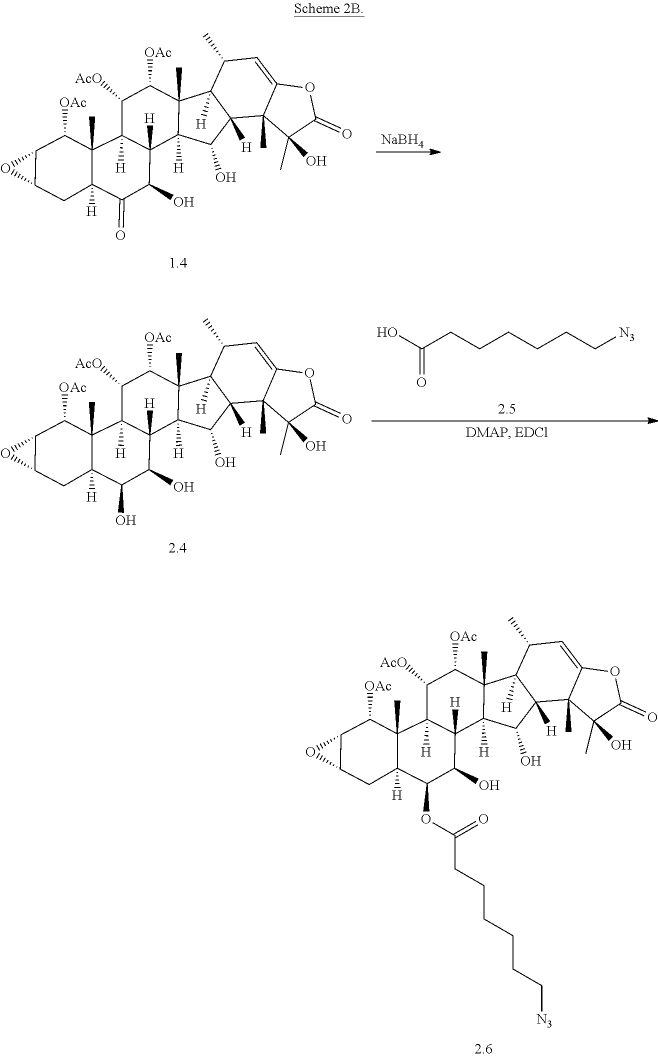

In one aspect, compounds of type 2.6, and similar compounds, can be prepared according to reaction Scheme 2B above. Thus, compounds of type 2.4 can be prepared by reduction of an appropriate ketone, e.g., 1.4 as shown above. Appropriate ketones are commercially available or prepared by methods known to one skilled in the art. The reduction is carried out in the presence of an appropriate reducing agent, e.g., sodium borohydride. Compounds of type 2.6 can be prepared by esterification of an appropriate alcohol, e.g., 2.4 as shown above, using an appropriate carboxylic acid, e.g., 2.5 as shown above. Appropriate carboxylic acids are commercially available or prepared by methods known to one skilled in the art. The esterification is carried out in the presence of an appropriate activating agent, e.g., 4-dimethylaminopyridine (DMAP), and an appropriate coupling agent, e.g., 1-ethyl-3-(3-dimethylaminopropyl)carbodiimide (EDCI). As can be appreciated by one skilled in the art, the above reaction provides an example of a generalized approach wherein compounds similar in structure to the specific reactants above (compounds similar to compounds of type 1.1 and 2.1), can be substituted in the reaction to provide substituted taccalonolide analogs similar to Formula 2.3.

3. Route III

In one aspect, taccalonolide analogs can be prepared as shown below.

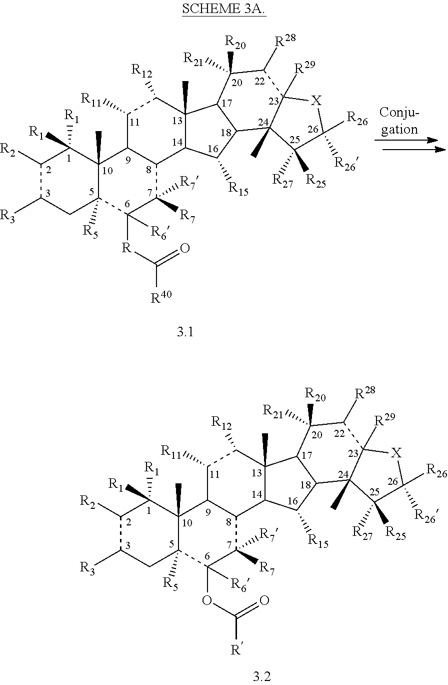

Compounds are represented in generic form, wherein R is O or NR⁴¹, wherein R′ is —(C1-C30 alkyl)-L-Z, —(C1-C30 alkyl)-L-(C1-C30 alkyl)-Z, or -L-(C1-C30)-Z, and with other substituents as noted in compound descriptions elsewhere herein. A more specific example is set forth below.

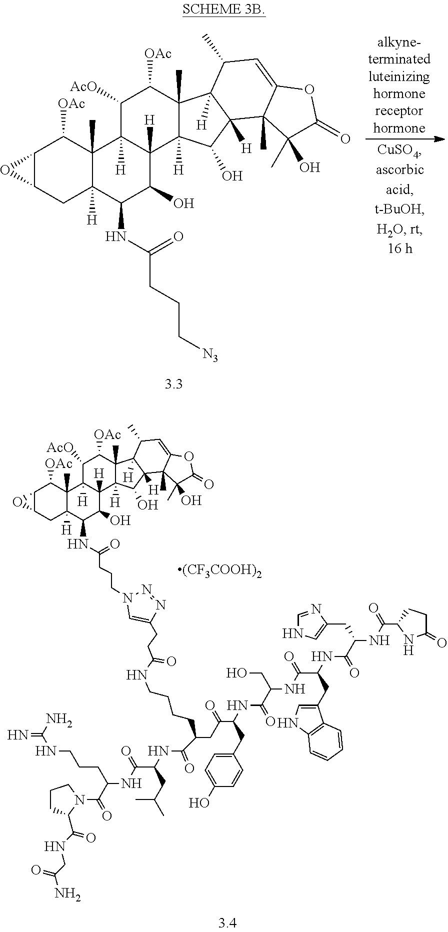

In one aspect, compounds of type 3.4, and similar compounds, can be prepared according to reaction Scheme 3B above. Thus, compounds of type 3.4 can be prepared by cyclization of an appropriate azide, e.g., 3.3 as shown above, and an appropriate alkyne, e.g., alkyne-terminated luteinizing hormone receptor hormone as shown above. Appropriate alkynes are commercially available or prepared by methods known to one skilled in the art. The cyclization is carried out in the presence of an appropriate catalyst, e.g., copper sulfate, and an appropriate acid, e.g., ascorbic acid, in an appropriate solvent system, e.g., t-butanol and water, for an appropriate period of time, e.g., 16 hours). As can be appreciated by one skilled in the art, the above reaction provides an example of a generalized approach wherein compounds similar in structure to the specific reactants above (compounds similar to compounds of type 3.3), can be substituted in the reaction to provide substituted taccalonolide analogs similar to Formula 3.4.

### E. Methods of Using the Compounds

The compounds and pharmaceutical compositions of the invention are useful as cellular probes (e.g., for the detection, visualization, and/or quantification of a target). The disclosed compounds are also useful in treating or controlling hyperproliferative disorders such as, for example, cardiovascular diseases such as, for example, coronary heart disease, stroke, hypertensive heart disease, inflammatory heart disease, and rheumatic heart disease, and cancers such as, for example, a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma).

Examples of cancers for which the compounds and compositions can be useful in treating, include, but are not limited to carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

In various aspects, further examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas.

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a cancer or of a fibrotic disorder.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a cancer, immune dysfunction, or a fibrotic disorder.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

1. Use as Cellular Probes

The compound disclosed herein are useful as cellular probes such as, for example, tubulin-labeling probes (e.g., fluorescence, biotin). Thus, in various aspects, a taccalonolide compound is covalently attached to an antibody, an antibody fragment, a vitamin, a hormone, a carbohydrate, a molecular ligand, an aptamer, a non-antibody protein, a peptide, a nucleic acid, a fluorophore, or a drug. In this way, a target can be “found,” allowing for detection, visualization, and/or quantification.

Thus, for example, in various aspects, a taccalonolide compound is covalently attached to a molecule that facilitates detection (e.g., fluorophore, biotin) to generate a probe. Such probes have found widespread application in the study of cell biological processes in the study of disease indications including, but not limited to, oncological disorders and hematological disorders. The development of probes can be challenging because many factors such as, for example, linkage stability, which can have a significant impact on targeting and detection. One major limitation of current small molecule tubulin-based fluorescent probes is their sensitivity to cold-induced microtubule depolymerization and drug efflux pumps. These needs and others are met by the instant invention.

Thus, in various aspects, disclosed is use of a compound as a cellular probe, wherein the compound has a structure represented by a formula:

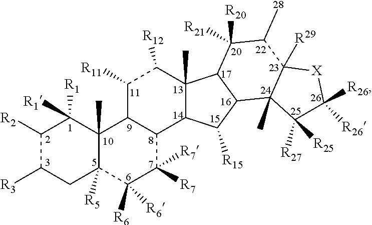

wherein each occurrence of - - - - - - is a single or double covalent bond; wherein X is selected from O, NR^{x} and C(R^{x})₂; wherein each occurrence of R^{x}, when present, is independently selected from hydrogen and C1-C6 alkyl; wherein R^{x} is selected from hydrogen, halogen, —OH, —CN, —NC, —NCO, —OCN, —NO2, —ONO₂, —ONO, —NO, —N3, —NH2, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹; wherein each occurrence of R³¹, R³², R³⁴, R^{35a}, and R^{35b}, when present, is independently selected from hydrogen and C1-C12 alkyl; wherein each occurrence of R³³, when present, is independently selected from hydrogen, C1-C12 alkyl, and monocyclic aryl monosubstituted with a methyl group; wherein each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and wherein R^{1′} is hydrogen; or wherein each of R¹ and R^{1′} together comprise ═O or ═NR³⁶; wherein each occurrence of R³⁶, when present, is independently selected from hydrogen and C1-C12 alkyl; wherein each of R² and R³ is independently selected from hydrogen, —OH, C1-C12 hydroxy, and halogen; or wherein each of R₂ and R₃ together comprise —O—; wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl); or wherein each of R⁷ and R^{7′} together comprise ═O; or wherein one of R⁷ and R^{7′} is absent; wherein each of R⁸ and R^{8′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr1, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, —NR⁴¹C(O)(C1-C30 alkyl)-L-Z, —NR⁴¹C(O)(C1-C30 alkyl)-L-(C1-C30 alkyl)-Z, and —NR⁴¹C(O)-L-(C1-C30 alkyl)-Z; wherein L is a linker; wherein Z is selected from an antibody and an antibody fragment; wherein each occurrence of R⁴¹ and R⁴², when present, is independently selected from hydrogen and C1-C12 alkyl; wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴; wherein each occurrence of Ar⁴, when present, is a structure represented by a formula:

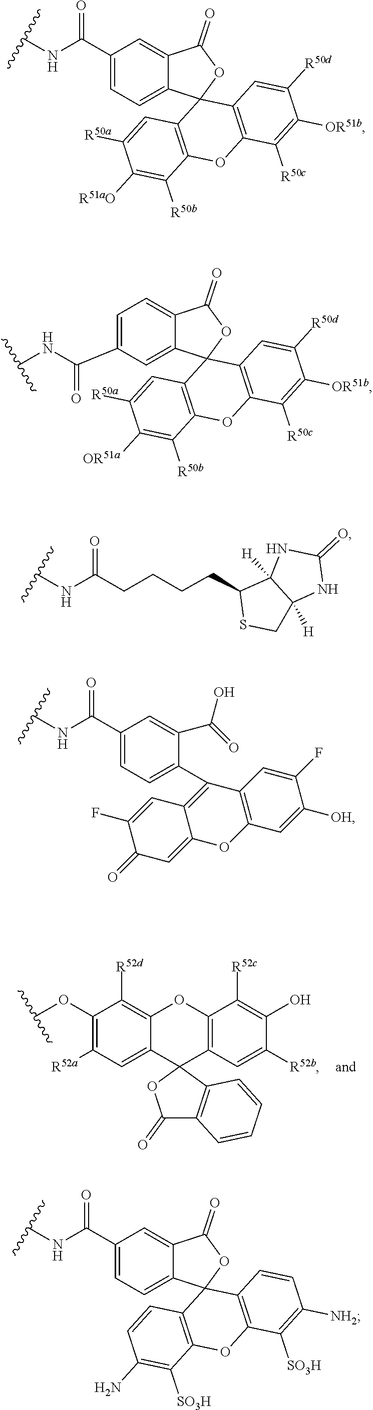

wherein each of R^{50a}, R^{50b}, R^{50c}, and R^{50d}, when present, is independently selected from hydrogen, —F, and —Cl; wherein each of R^{51a} and R^{51b}, when present, is independently selected from hydrogen and —C(O)(C1-C12 alkyl); wherein each of R^{52a}, R^{52b}, R^{52c}, and R^{52d}, when present, is independently selected from hydrogen, —F, and —Cl; wherein each occurrence of Ar³, when present, is a structure represented by a formula selected from:

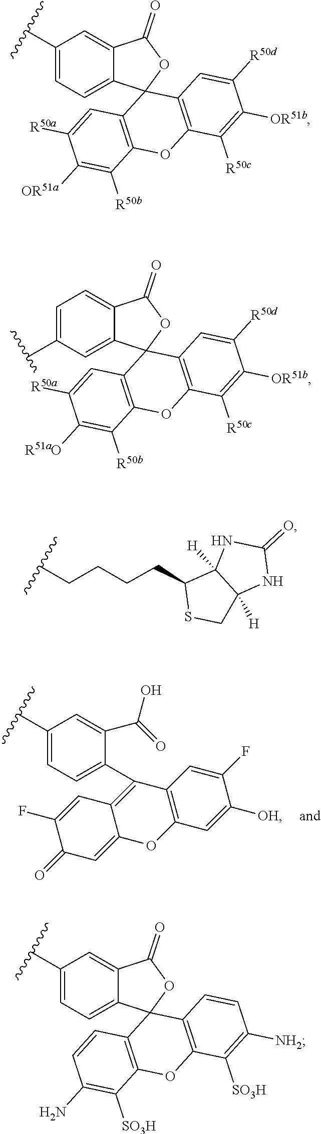

or wherein one of R⁸ and R^{8′} is absent; wherein each of R¹¹ and R¹² is independently selected from hydrogen, —OH, C1-C8 hydroxy, C1-C6 alkyl, C1-C8 alkoxy, and —OC(O)(C1-C8 alkyl); wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide); wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R²¹ is selected from hydrogen and C1-C6 alkyl; wherein R²¹ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide); wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each of R²⁶ and R^{26′} is independently selected from hydrogen, —OH, C1-C8 hydroxy, and C1-C8 alkoxy; or wherein each of R²⁶ and R^{26′} together comprise ═O; wherein R²⁷ is selected from hydrogen and C1-C6 alkyl; wherein each of R²⁸ and R²⁹ is independently selected from hydrogen and halogen; or wherein each of R²⁸ and R²⁹ together comprise —O— or —N(R³⁷)—; wherein R³⁷, when present, is selected from hydrogen, C1-C4 alkyl, —SO₂R⁷¹, and a structure having a formula:

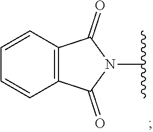 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that one and only one of R⁸ and R^{8′} is —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl)-L-Z, —NR⁴¹C(O)(C1-C30 alkyl)-L-(C1-C30 alkyl)-Z, or —NR⁴¹C(O)-L-(C1-C30 alkyl)-Z, and provided that when one or both of R²⁸ and R²⁹ is hydrogen then the occurrence of - - - - - - at C-22/C-23 is a double covalent bond, or a pharmaceutically acceptable salt thereof.

In a further aspect, disclosed is use of a compound as an antibody-drug conjugate.

2. Treatment Methods

The compounds disclosed herein are useful for treating or controlling hyperproliferative disorders such as, for example, cardiovascular diseases such as, for example, coronary heart disease, stroke, hypertensive heart disease, inflammatory heart disease, and rheumatic heart disease, and cancers such as, for example, a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma). Thus, provided is a method comprising administering a therapeutically effective amount of a composition comprising a disclosed compound to a subject. In a further aspect, the method can be a method for treating cancer. In a still further aspect, the method can be a method for treating a cardiovascular disease.

a. Treating Hyperproliferative Disorders

In one aspect, disclosed are methods of treating a hyperproliferative disorder in a mammal, the method comprising the step of administering to the mammal an effective amount of at least one disclosed compound, or a pharmaceutically acceptable salt thereof.

In one aspect, disclosed are methods for the treatment of a hyperproliferative disorder in a subject, the method comprising administering to the subject an effective amount of at least one compound having a structure represented by a formula:

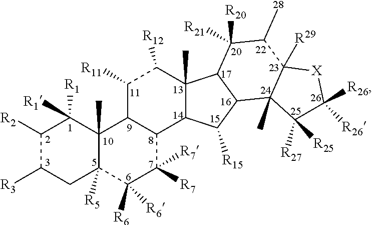

wherein each occurrence of - - - - - - is a single or double covalent bond; wherein X is selected from O, NR^{x}, and C(R^{x})₂; wherein each occurrence of R^{x}, when present, is independently selected from hydrogen and C1-C6 alkyl; wherein R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹; wherein each occurrence of R³¹, R³², R³⁴, R^{35a}, and R^{35b}, when present, is independently selected from hydrogen and C1-C12 alkyl; wherein each occurrence of R³³, when present, is independently selected from hydrogen, C1-C12 alkyl, and monocyclic aryl monosubstituted with a methyl group; wherein each occurrence of Cy1, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and wherein R¹ is hydrogen; or wherein each of R¹ and R^{1′} together comprise ═O or ═NR³⁶; wherein each occurrence of R³⁶, when present, is independently selected from hydrogen and C1-C12 alkyl; wherein each of R² and R³ is independently selected from hydrogen, —OH, C1-C12 hydroxy, and halogen; or wherein each of R₂ and R₃ together comprise —O—; wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; wherein each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, (C1-C30 alkyl)OC(O)NR^{35a}R^{35b}, Cy¹, Ar¹, —(C1-C30 alkyl)Ar1, —OAr1, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)R⁴⁰, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰; wherein each occurrence of R⁴⁰, when present, is independently a C1-C30 alkyl functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide; wherein each occurrence of R⁴¹ and R⁴², when present, is independently selected from hydrogen and C1-C12 alkyl; wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴; wherein each occurrence of Ar⁴, when present, is a structure represented by a formula selected from:

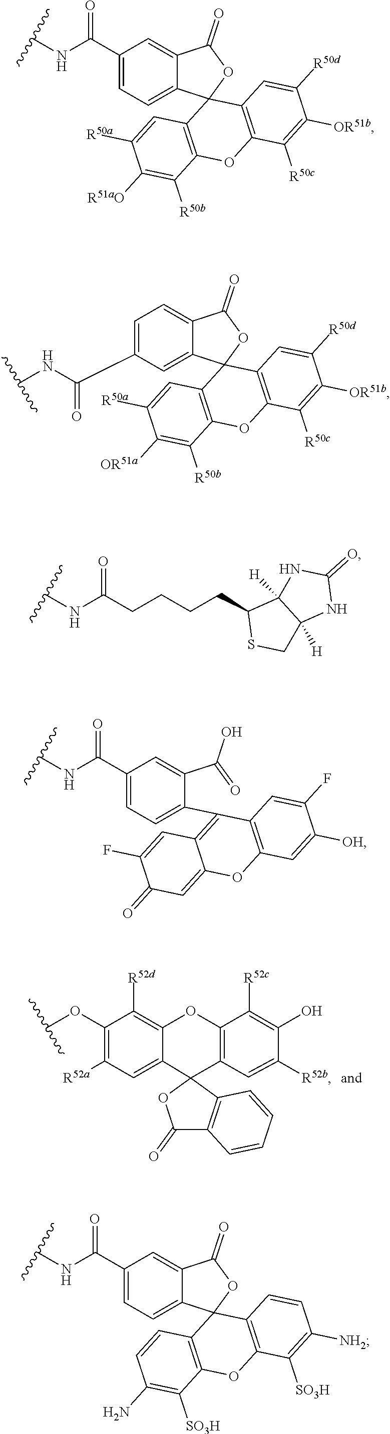

wherein each of R^{50a}, R^{50b}, R^{50c}, and R^{50d}, when present, is independently selected from hydrogen, —F, and —Cl; wherein each of R^{51a} and R^{51b}, when present, is independently selected from hydrogen and —C(O)(C1-C12 alkyl); wherein each of R^{52a}, R^{52b}, R^{52c}, and R^{52d}, when present, is independently selected from hydrogen, —F, and —Cl; wherein each occurrence of Ar³, when present, is a structure represented by a formula selected from:

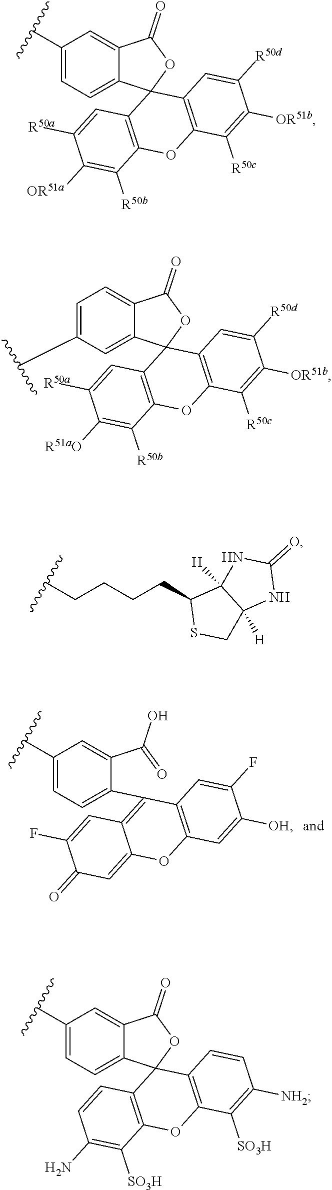

or wherein one of R⁶ and R^{6′} is absent; wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl); or wherein each of R⁷ and R^{7′} together comprise ═O; or wherein one of R⁷ and R^{7′} is absent; wherein each of R¹¹ and R¹² is independently selected from hydrogen, —OH, C1-C8 hydroxy, C1-C6 alkyl, C1-C8 alkoxy, and —OC(O)(C1-C8 alkyl); wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide); wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R²¹ is selected from hydrogen and C1-C6 alkyl; wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide); wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each of R²⁶ and R^{26′} is independently selected from hydrogen, —OH, C1-C8 hydroxy, and C1-C8 alkoxy; or wherein each of R²⁶ and R^{26′} together comprise ═O; wherein R²⁷ is selected from hydrogen and C1-C6 alkyl; wherein each of R²⁸ and R²⁹ is independently selected from hydrogen and halogen; or wherein each of R²⁸ and R²⁹ together comprise —O— or —N(R³⁷)—; wherein R³⁷, when present, is selected from hydrogen, C1-C4 alkyl, —SO₂R⁷¹, and a structure having a formula:

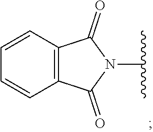 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that at least one of R⁶ and R^{6′} is —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, or —NR⁴¹C(O)R⁴⁰, and provided that when one or both of R²⁸ and R²⁹ is hydrogen then the occurrence of - - - - - - at C-22/C-23 is a double covalent bond, or a pharmaceutically acceptable salt thereof.

In one aspect, disclosed are methods for the treatment of a hyperproliferative disorder in a subject, the method comprising administering to the subject an effective amount of at least one compound having a structure represented by a formula:

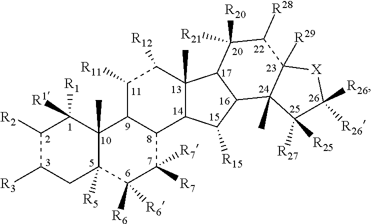

wherein each occurrence of - - - - - - is a single or double covalent bond; wherein X is selected from O, NR^{x} and C(R^{x})₂; wherein each occurrence of R^{x}, when present, is independently selected from hydrogen and C1-C6 alkyl; wherein R¹ is selected from hydrogen, halogen, —OH, —CN, —NC, —NCO, —OCN, —NO₂, —ONO₂, —ONO, —NO, —N₃, —NH₂, —NH₃, —N═NR³¹, N HOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹; wherein each occurrence of R³¹, R³², R³⁴, R^{35a}, and R^{35b}, when present, is independently selected from hydrogen and C1-C12 alkyl; wherein each occurrence of R³³, when present, is independently selected from hydrogen, C1-C12 alkyl, and monocyclic aryl monosubstituted with a methyl group; wherein each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and wherein R^{1′} is hydrogen; or wherein each of R¹ and R^{1′} together comprise ═O or ═NR³⁶; wherein each occurrence of R³⁶, when present, is independently selected from hydrogen and C1-C12 alkyl; wherein each of R² and R³ is independently selected from hydrogen, —OH, C1-C12 hydroxy, and halogen; or wherein each of R₂ and R₃ together comprise —O—; wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; wherein each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, (C1-C30 alkyl)OC(O)NR^{35a}R^{35b}, Cy1, Ar1, —(C1-C30 alkyl)Ar¹, —OAr1, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)R⁴⁰, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰; wherein each occurrence of R⁴⁰, when present, is independently a C1-C30 alkyl functionalized with a group selected from —SH, —NH₂, —NHOH, terminal alkyne, haloacetyl, maleimide, isothiocyanate, N-hydroxysuccinimde, succinimidyl ester, tetrafluorophenyl ester, sulfodichlorophenol ester, and hydrazide; wherein each occurrence of R⁴¹ and R⁴², when present, is independently selected from hydrogen and C1-C12 alkyl; wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴; wherein each occurrence of Ar⁴, when present, is a structure represented by a formula:

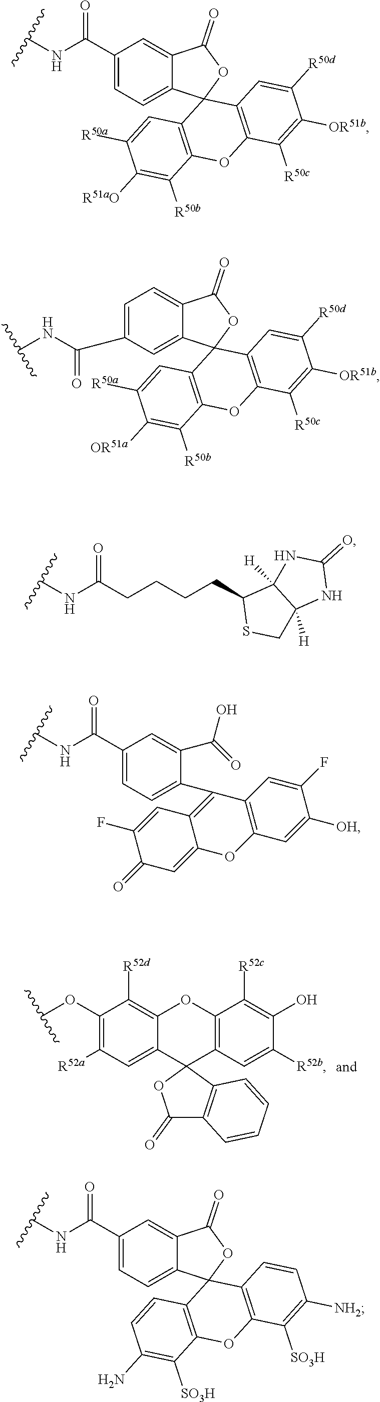

wherein each of R^{50a}, R^{50b}, R^{50c}, and R^{50d}, when present, is independently selected from hydrogen, —F, and —Cl; wherein each of R^{51a} and R^{51b}, when present, is independently selected from hydrogen and —C(O)(C1-C12 alkyl); wherein each of R^{52a}, R^{52b}, R^{52c}, and R^{52d}, when present, is independently selected from hydrogen, —F, and —Cl; wherein each occurrence of Ar³, when present, is a structure represented by a formula selected from:

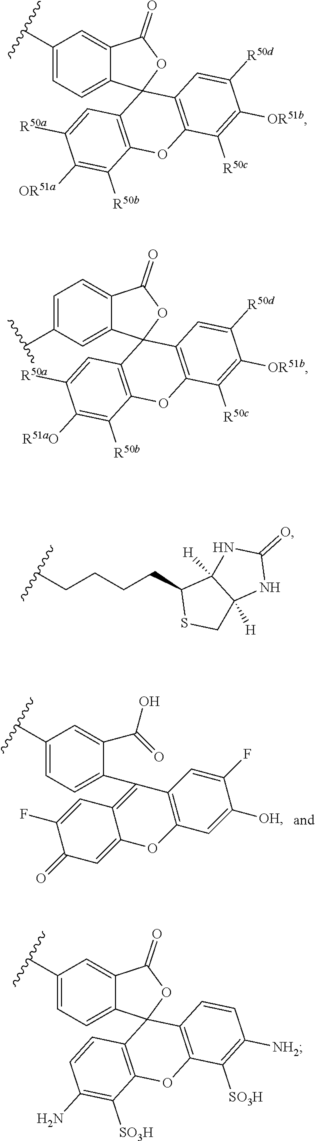

or wherein one of R⁶ and R^{6′} is absent; wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl); or wherein each of R⁷ and R^{7′} together comprise ═O; or wherein one of R⁷ and R^{7′} is absent; wherein each of R¹¹ and R¹² is independently selected from hydrogen, —OH, C1-C8 hydroxy, C1-C6 alkyl, C1-C8 alkoxy, and —OC(O)(C1-C8 alkyl); wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide); wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R²¹ is selected from hydrogen and C1-C6 alkyl; wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide); wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each of R²⁶ and R^{26′} is independently selected from hydrogen, —OH, C1-C8 hydroxy, and C1-C8 alkoxy; or wherein each of R²⁶ and R^{26′} together comprise ═O; wherein R²⁷ is selected from hydrogen and C1-C6 alkyl; wherein each of R²⁸ and R²⁹ is independently selected from hydrogen and halogen; or wherein each of R²⁸ and R²⁹ together comprise —O— or —N(R³⁷)—; wherein R³⁷, when present, is selected from hydrogen, C1-C4 alkyl, —SO₂R⁷¹, and a structure having a formula:

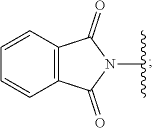 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that at least one of R⁶ and R^{6′} is —OC(O)R⁴⁰ or —NR⁴¹C(O)R⁴⁰, and provided that when one or both of R²⁸ and R²⁹ is hydrogen then the occurrence of - - - - - - at C-22/C-23 is a double covalent bond, or a pharmaceutically acceptable salt thereof.

In one aspect, disclosed are methods for the treatment of a hyperproliferative disorder in a subject, the method comprising administering to the subject an effective amount of at least one compound having a structure represented by a formula:

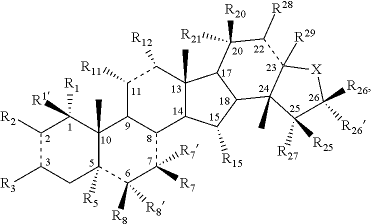

wherein each occurrence of - - - - - - is a single or double covalent bond; wherein X is selected from O, NR^{x} and C(R^{x})₂; wherein each occurrence of R^{x}, when present, is independently selected from hydrogen and C1-C6 alkyl; wherein R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³¹, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, (C1-C12 alkyl)OC(O)NR^{35a}R^{35b}, Cy¹, Ar¹, (C1-C12 alkyl)Ar¹, and —OAr¹; wherein each occurrence of R³¹, R³², R³⁴, R^{35a}, and R^{35b}, when present, is independently selected from hydrogen and C1-C12 alkyl; wherein each occurrence of R³³, when present, is independently selected from hydrogen, C1-C12 alkyl, and monocyclic aryl monosubstituted with a methyl group; wherein each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and wherein R^{1′} is hydrogen; or wherein each of R¹ and R^{1′} together comprise ═O or ═NR³⁶; wherein each occurrence of R³⁶, when present, is independently selected from hydrogen and C1-C12 alkyl; wherein each of R² and R³ is independently selected from hydrogen, —OH, C1-C12 hydroxy, and halogen; or wherein each of R₂ and R₃ together comprise —O—; wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino; or wherein R⁵ is absent; wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl); or wherein each of R⁷ and R^{7′} together comprise ═O; or wherein one of R⁷ and R^{7′} is absent; wherein each of R⁸ and R^{8′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr1, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, —NR⁴¹C(O)(C1-C30 alkyl)-L-Z, —NR⁴¹C(O)(C1-C30 alkyl)-L-(C1-C30 alkyl)-Z, and —NR⁴¹C(O)-L-(C1-C30 alkyl)-Z; wherein L is a linker; wherein Z is selected from an antibody, an antibody fragment, a vitamin, a hormone, a carbohydrate, a molecular ligand, an aptamer, a non-antibody protein, a peptide, a nucleic acid, a fluorophore, and a drug; wherein each occurrence of R⁴¹ and R⁴², when present, is independently selected from hydrogen and C1-C12 alkyl; wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴; wherein each occurrence of Ar⁴, when present, is a structure represented by a formula:

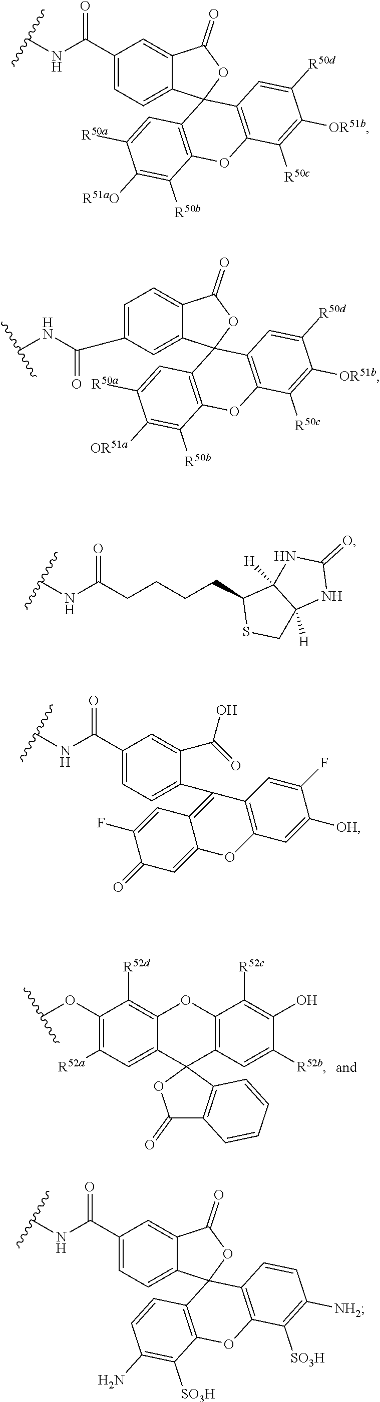

wherein each of R^{50a}, R^{50b}, R^{50c}, and R^{50d}, when present, is independently selected from hydrogen, —F, and —Cl; wherein each of R^{51a} and R^{51b}, when present, is independently selected from hydrogen and —C(O)(C1-C12 alkyl); wherein each of R^{52a}, R^{52b}, R^{52c}, and R^{52d}, when present, is independently selected from hydrogen, —F, and —Cl; wherein each occurrence of Ar³, when present, is a structure represented by a formula selected from:

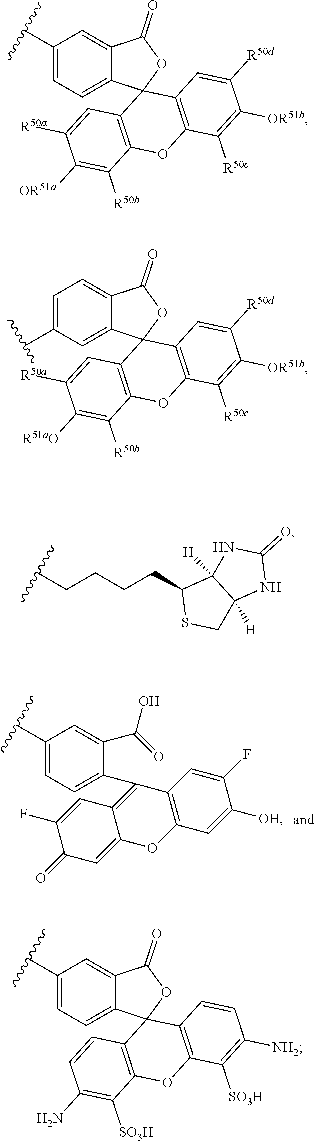

or wherein one of R⁸ and R^{8′} is absent; wherein each of R¹¹ and R¹² is independently selected from hydrogen, —OH, C1-C8 hydroxy, C1-C6 alkyl, C1-C8 alkoxy, and —OC(O)(C1-C8 alkyl); wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar2, —OC(O)(C1-C4 alkyl)Ar², and —OC(O)(C1-C8 azide); wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl); wherein R²¹ is selected from hydrogen and C1-C6 alkyl; wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide); wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein each of R²⁶ and R^{26′} is independently selected from hydrogen, —OH, C1-C8 hydroxy, and C1-C8 alkoxy; or wherein each of R²⁶ and R^{26′} together comprise ═O; wherein R²⁷ is selected from hydrogen and C1-C6 alkyl; wherein each of R²⁸ and R²⁹ is independently selected from hydrogen and halogen; or wherein each of R²⁸ and R²⁹ together comprise —O— or N(R37 wherein R³⁷, when present, is selected from hydrogen, C1-C4 alkyl, —SO₂R⁷¹, and a structure having a formula:

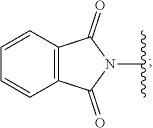 [SMILES: *N1C(=O)c2ccccc2C1=O]

and
wherein R⁷¹, when present, is selected from hydrogen, C1-C4 alkyl, —CH₂CH₂Si(CH₃)₃, and monocyclic aryl monosubstituted with a methyl group, provided that one and only one of R⁸ and R^{8′} is —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl)-L-Z, —NR⁴¹C(O)(C1-C30 alkyl)-L-(C1-C30 alkyl)-Z, or —NR⁴¹C(O)-L-(C1-C30 alkyl)-Z, and provided that when one or both of R²⁸ and R²⁹ is hydrogen then the occurrence of - - - - - - at C-22/C-23 is a double covalent bond, or a pharmaceutically acceptable salt thereof.

In one aspect, disclosed are methods for the treatment of a hyperproliferative disorder in a subject, the method comprising administering to the subject an effective amount of at least one compound having a structure selected from:

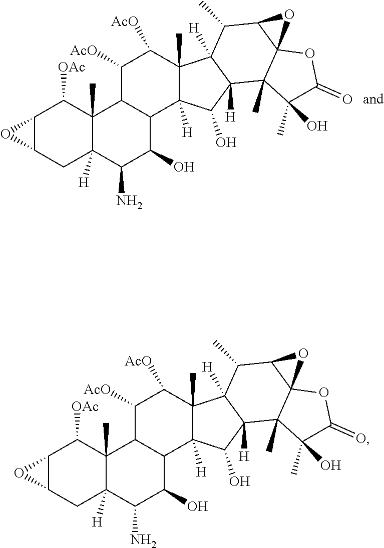 [SMILES: CC(=O)O[C@H]1C2C([C@@H](O)[C@@H](N)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O ; CC(=O)O[C@H]1C2C([C@@H](O)[C@H](N)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

or a pharmaceutically acceptable salt thereof.

In one aspect, disclosed are methods for the treatment of a hyperproliferative disorder in a subject, the method comprising administering to the subject an effective amount of at least one compound selected from:

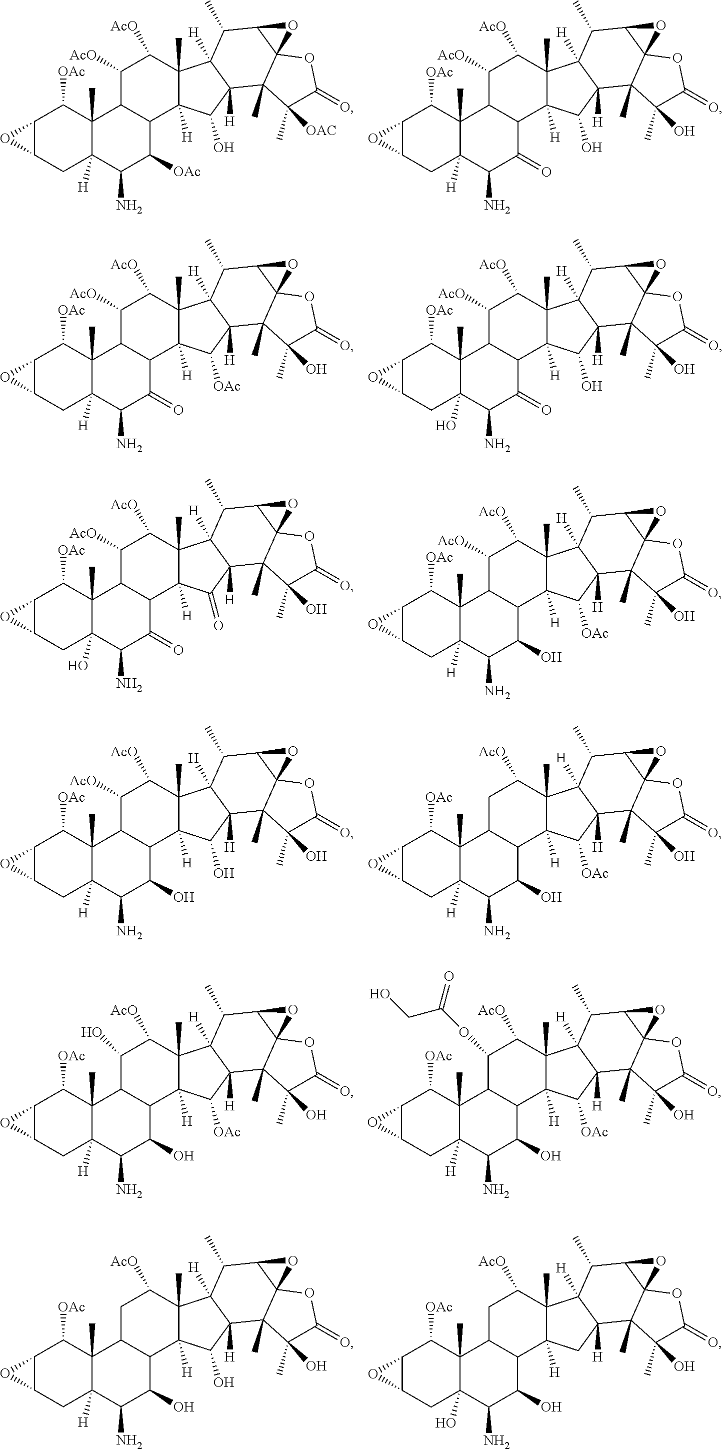

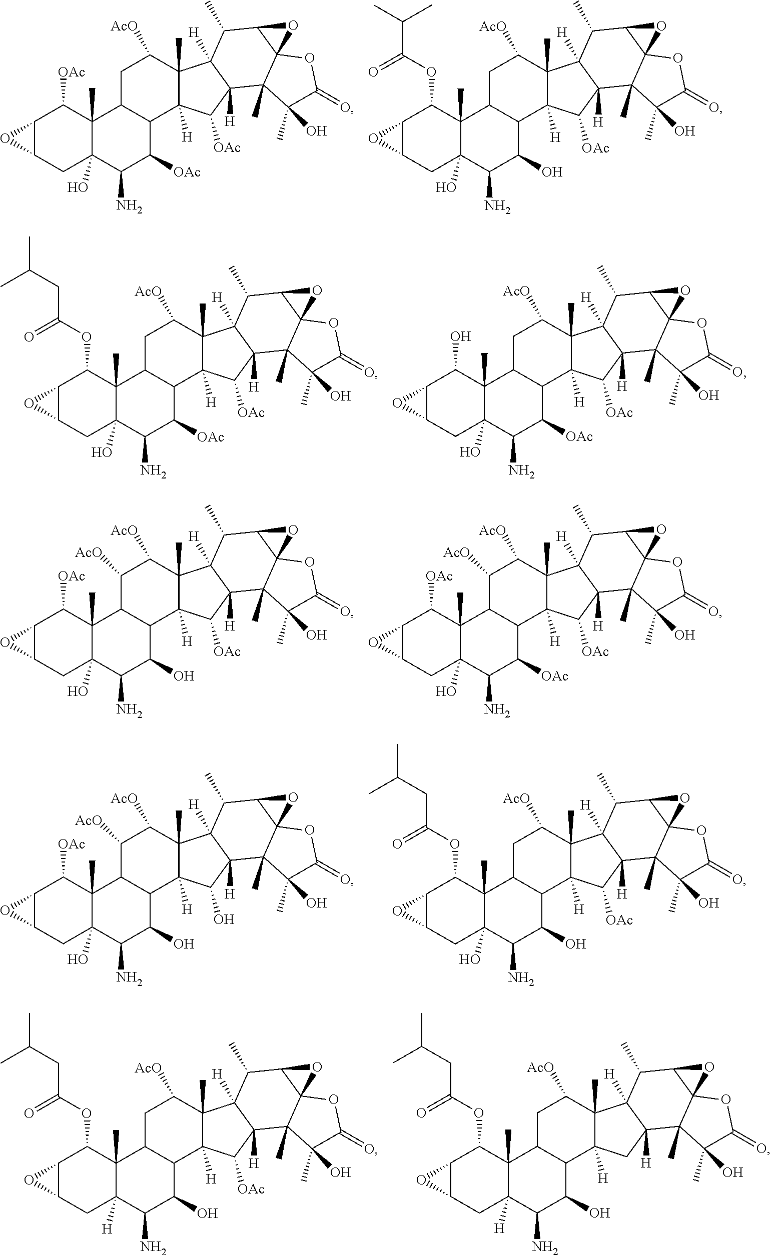

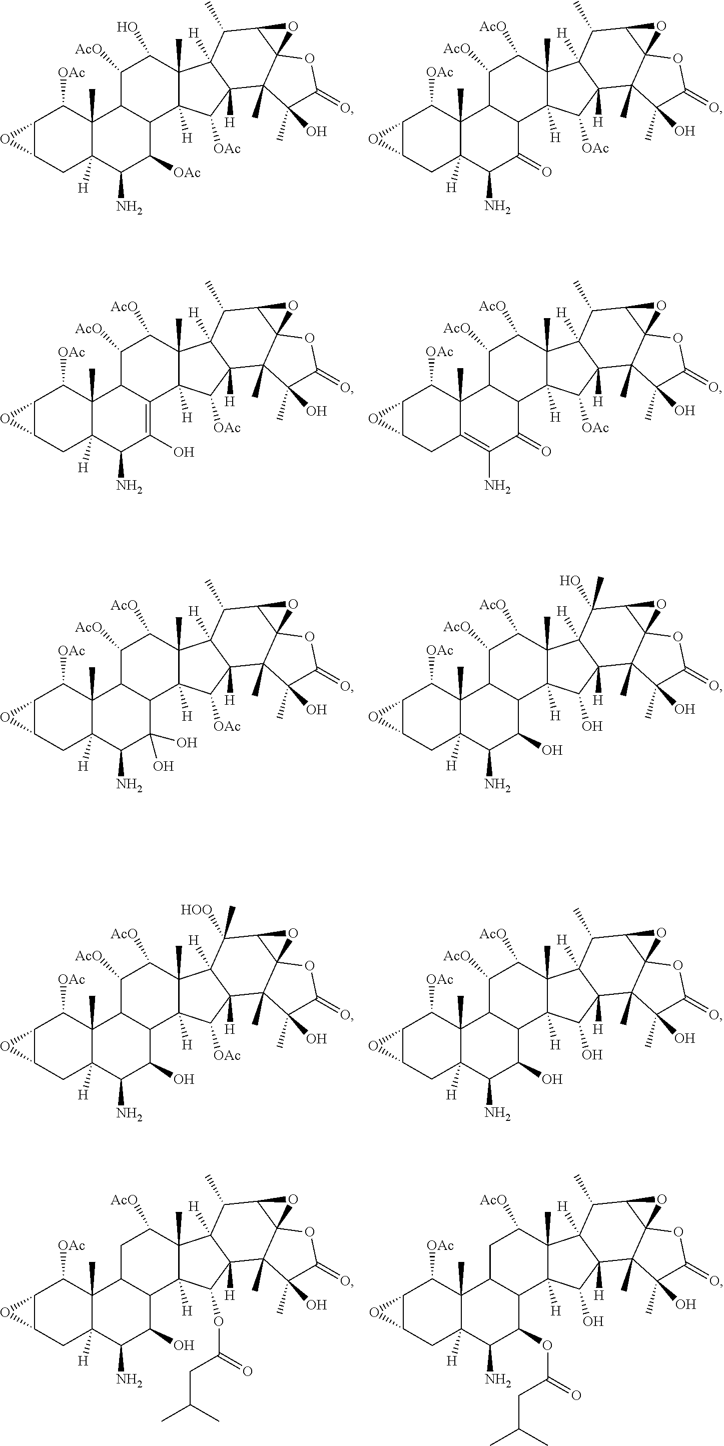

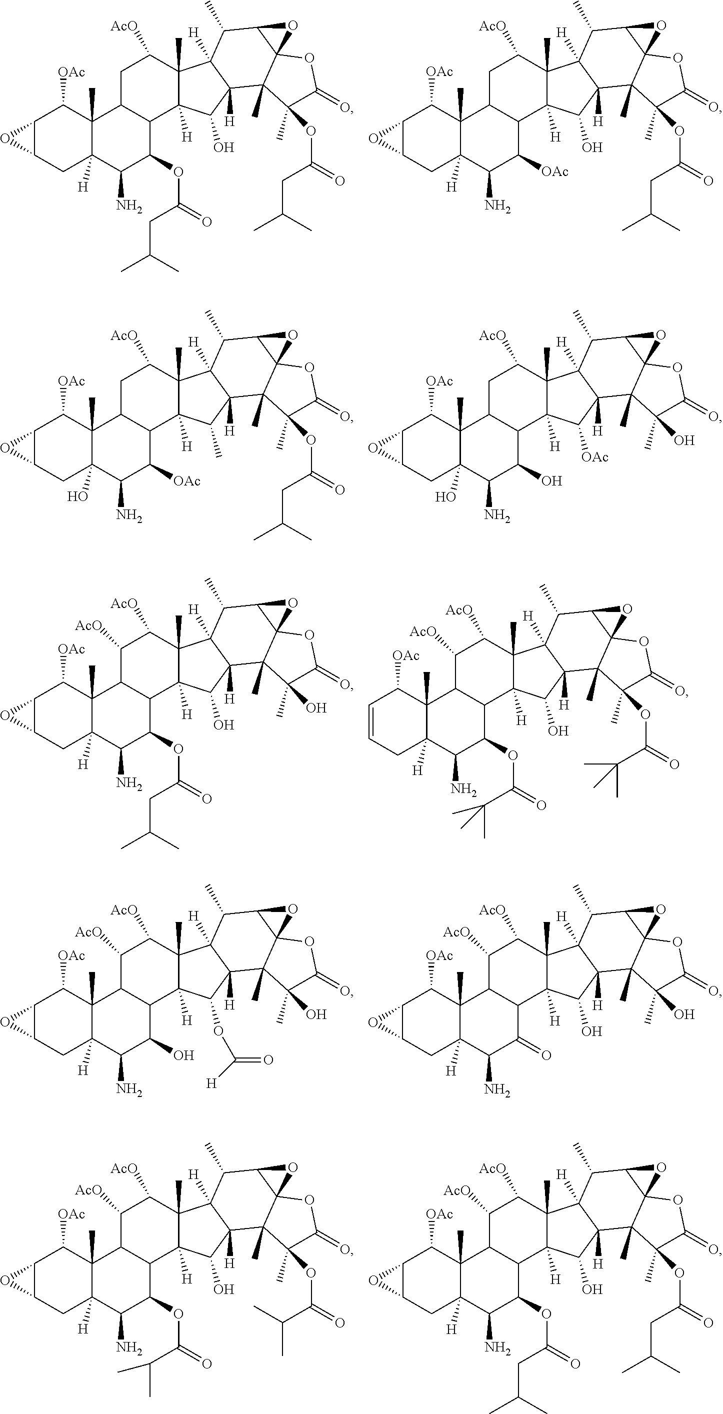

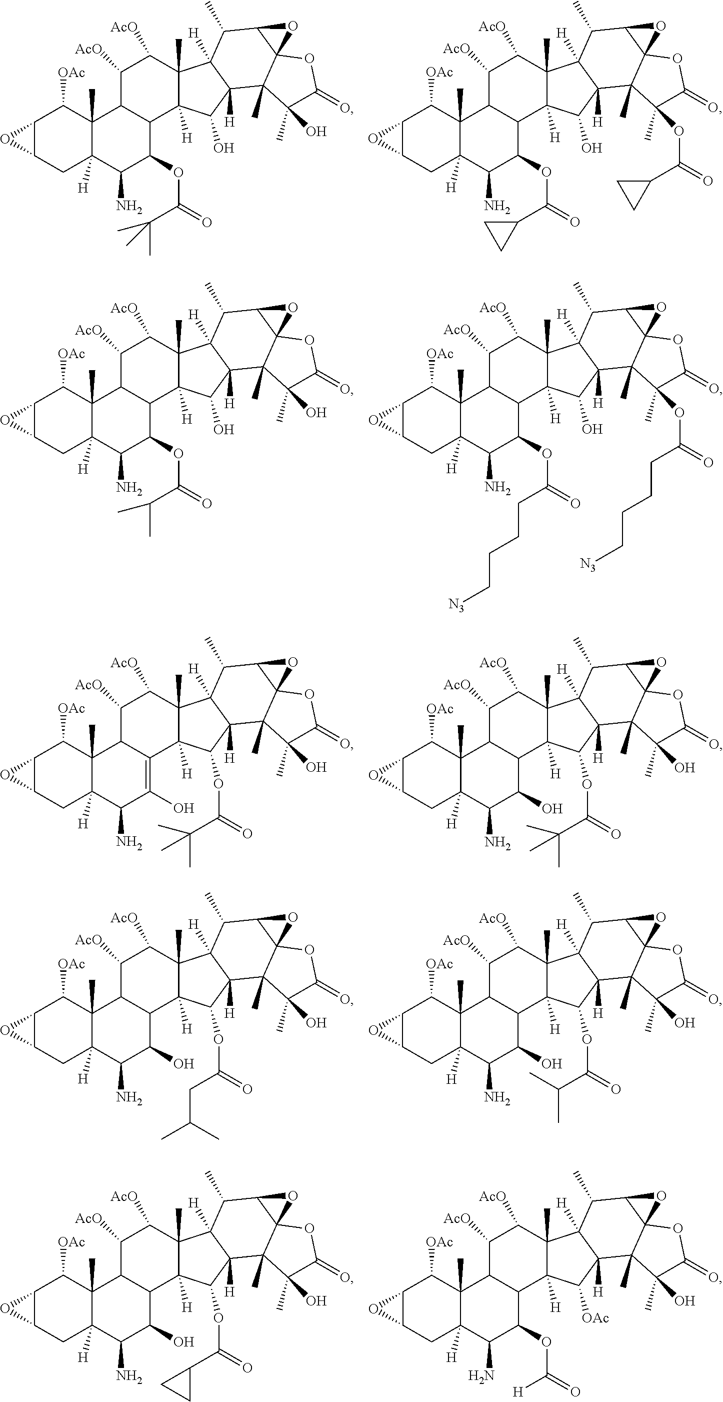

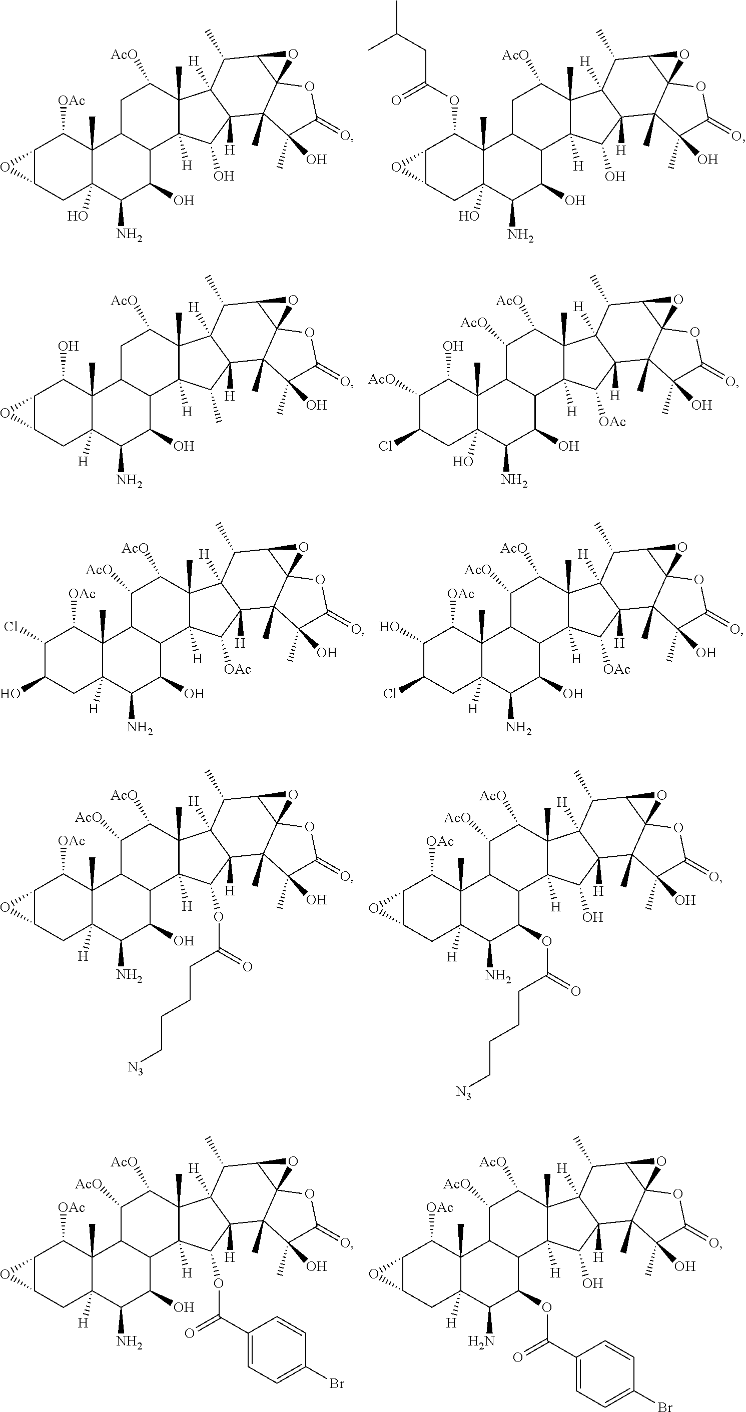

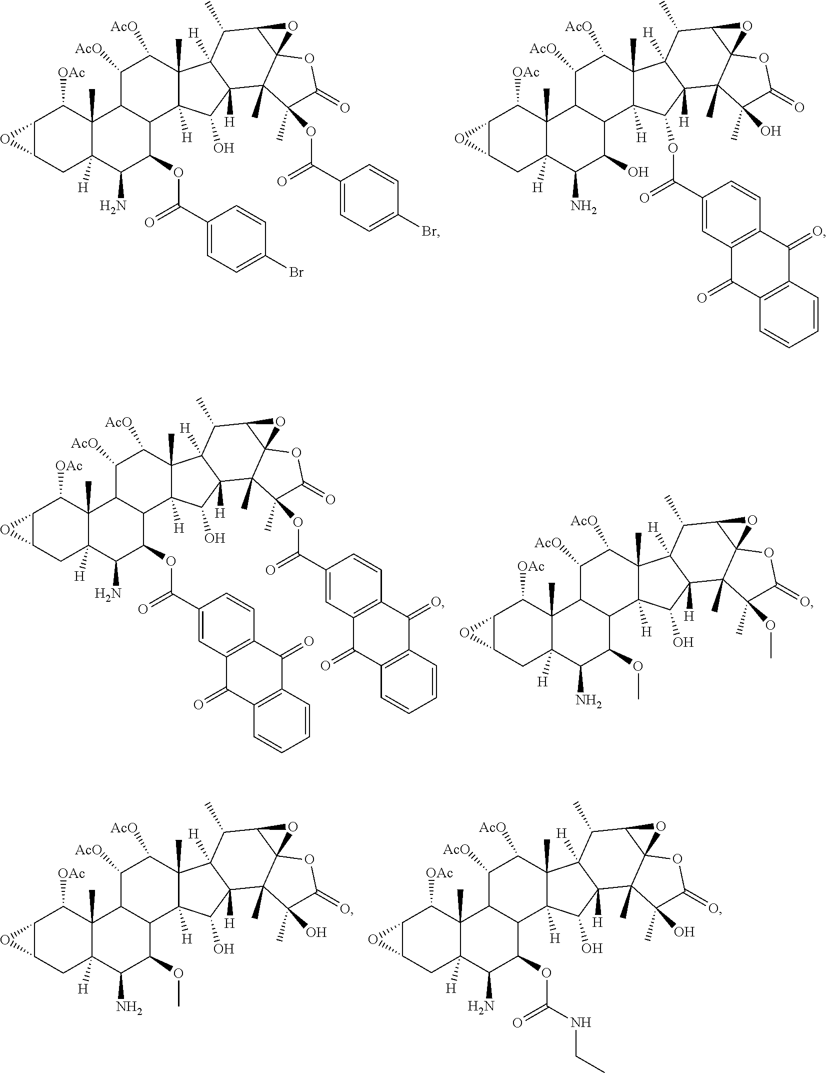

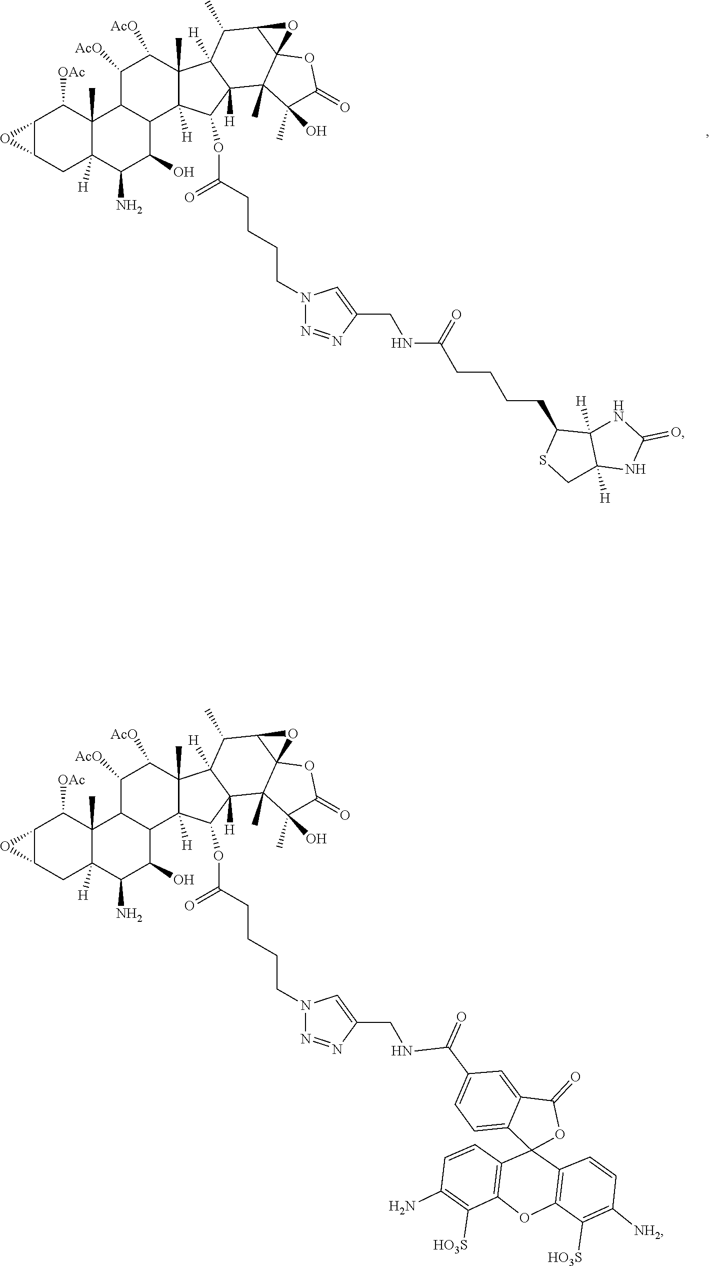

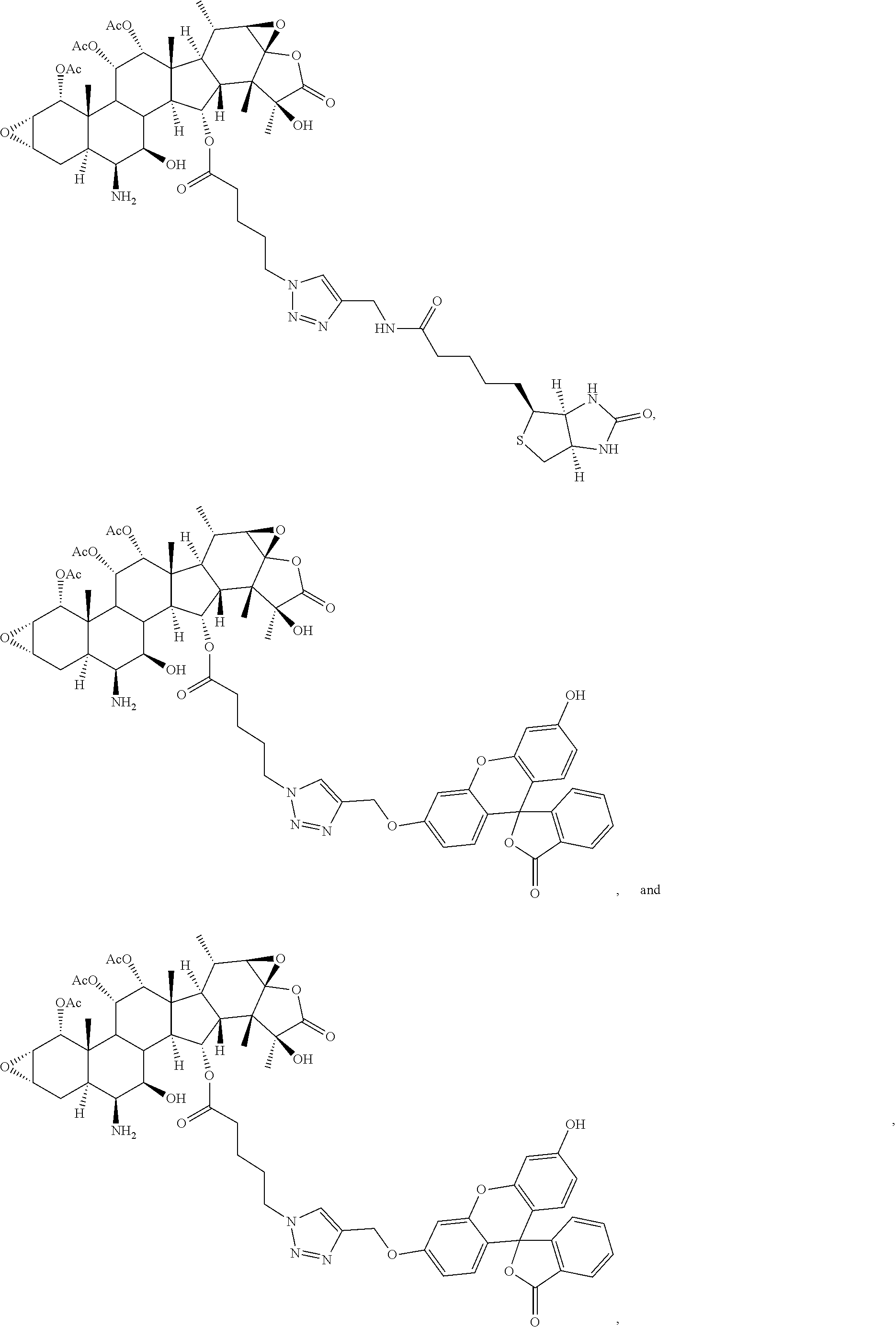

or a pharmaceutically acceptable salt thereof.

In one aspect, disclosed are methods for the treatment of a hyperproliferative disorder in a subject, the method comprising administering to the subject an effective amount of at least one compound having a structure selected from:

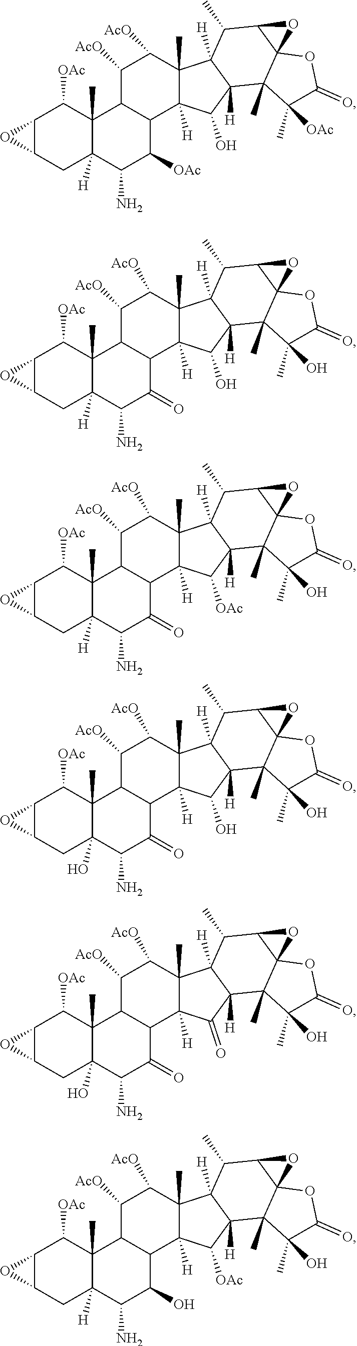

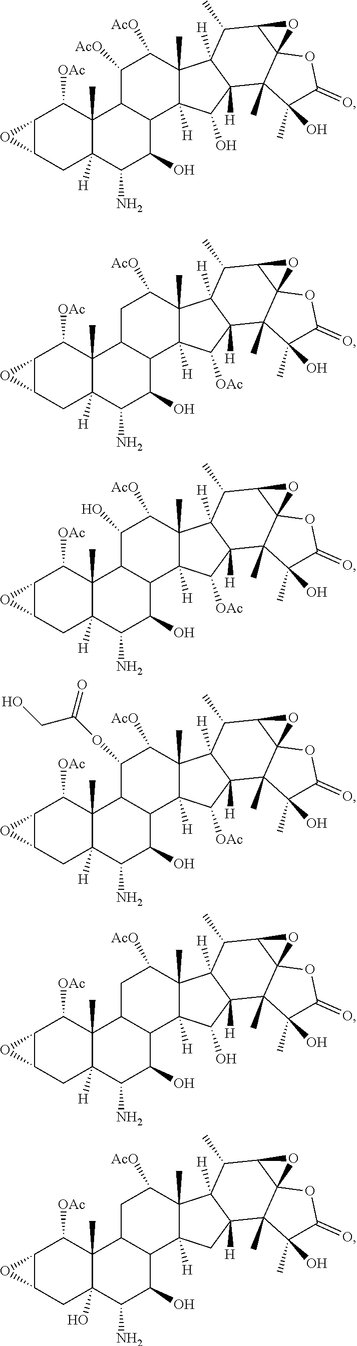

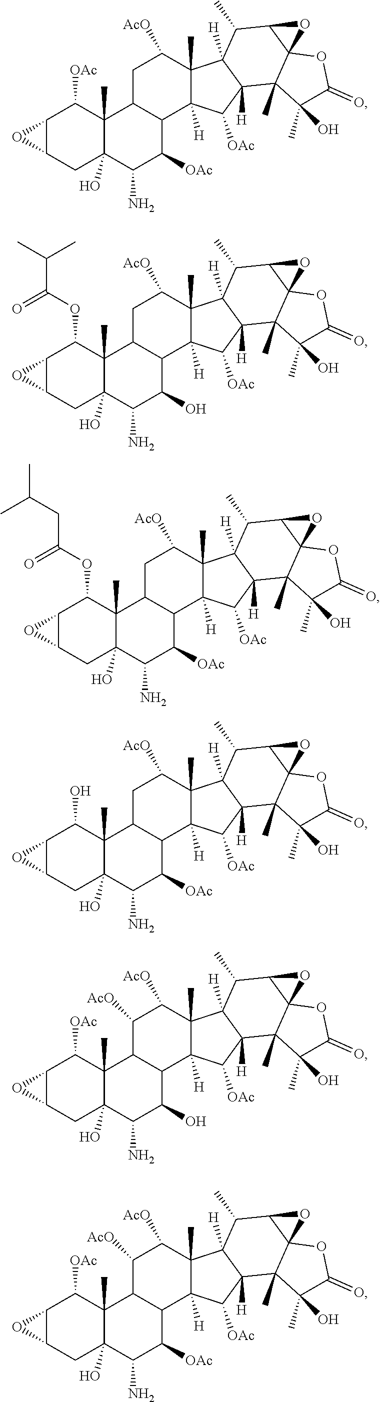

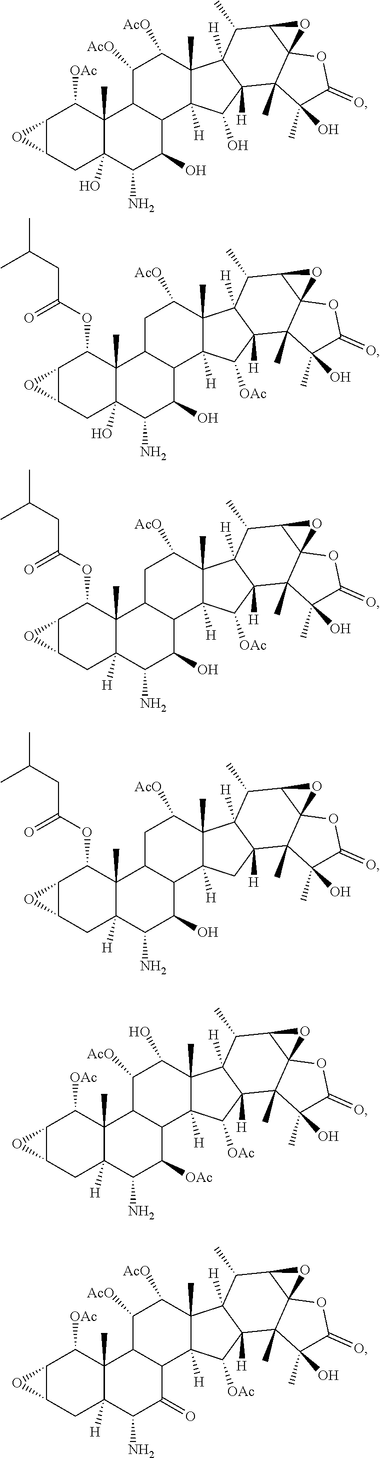

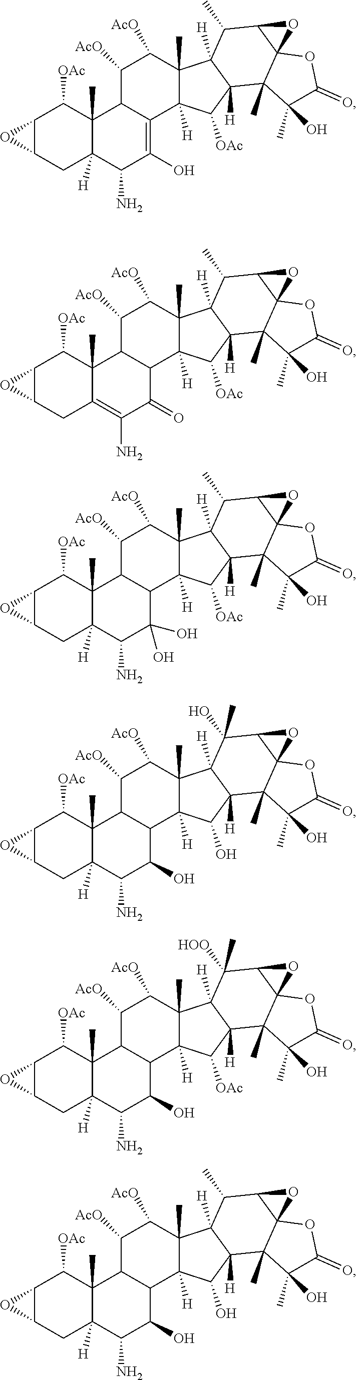

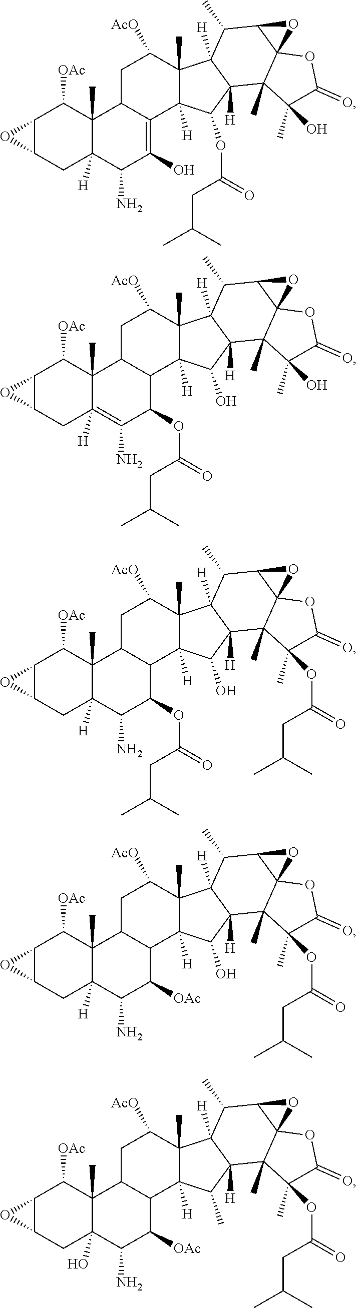

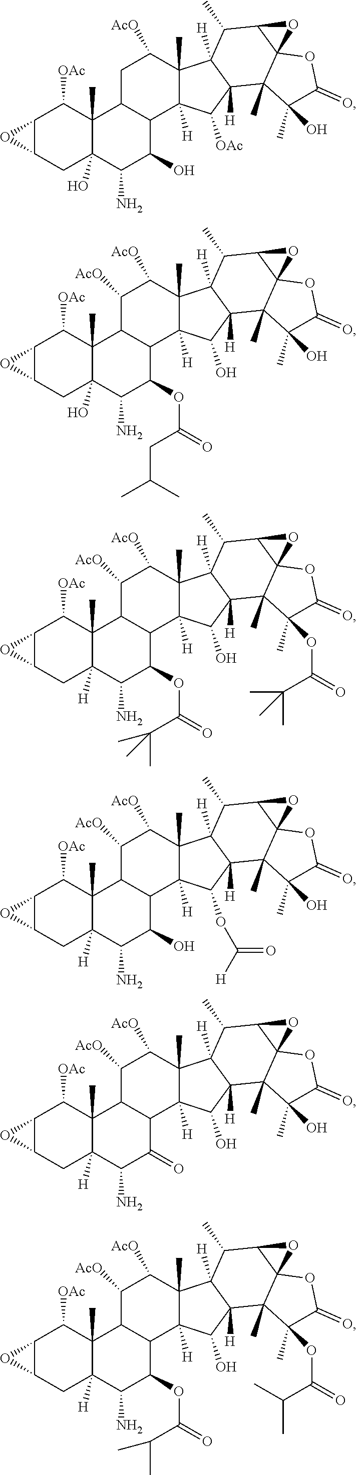

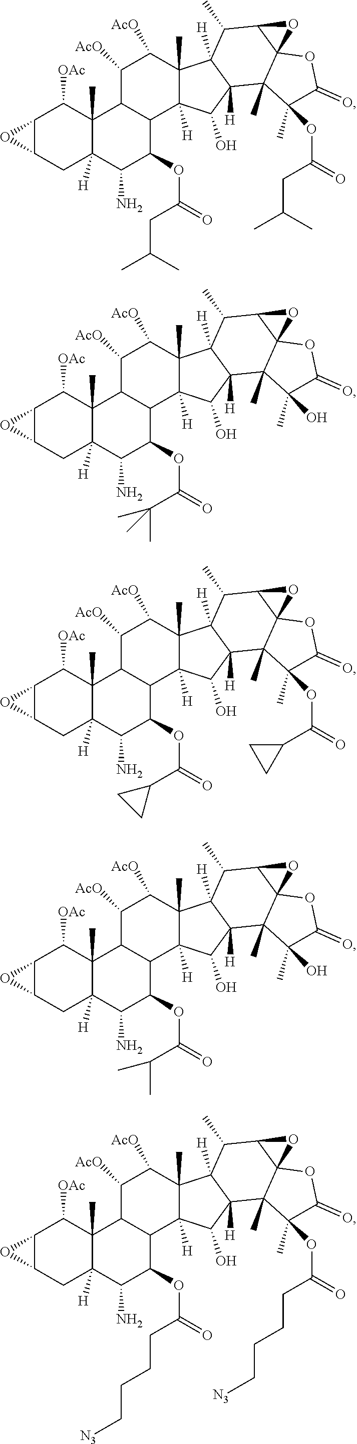

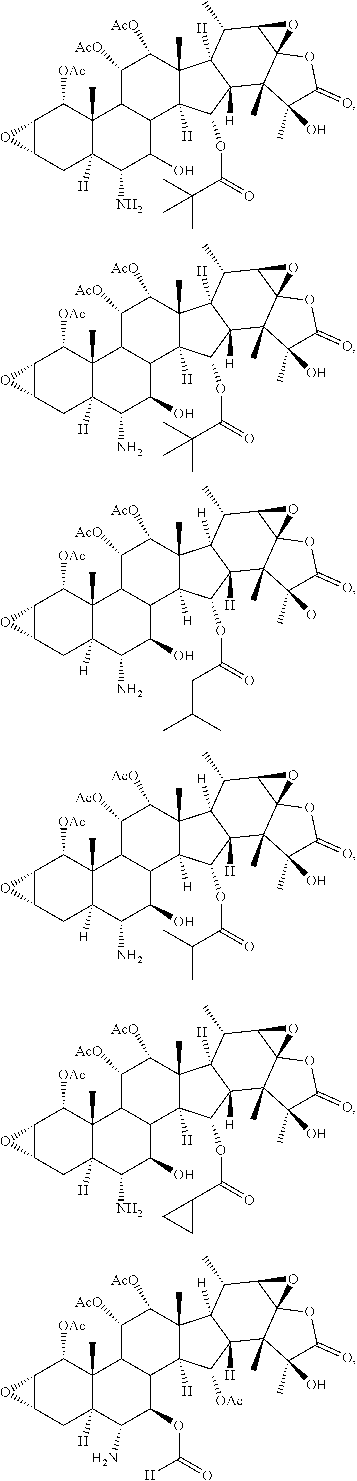

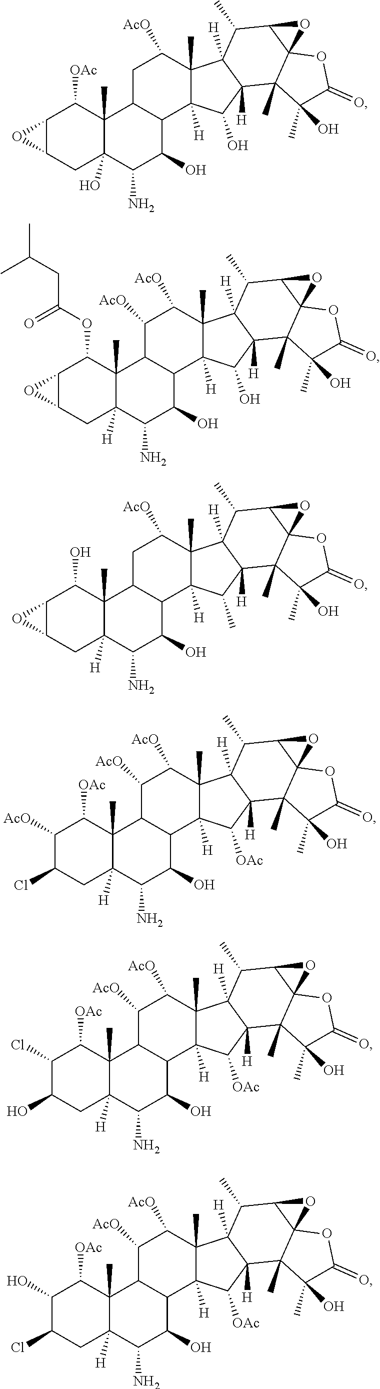

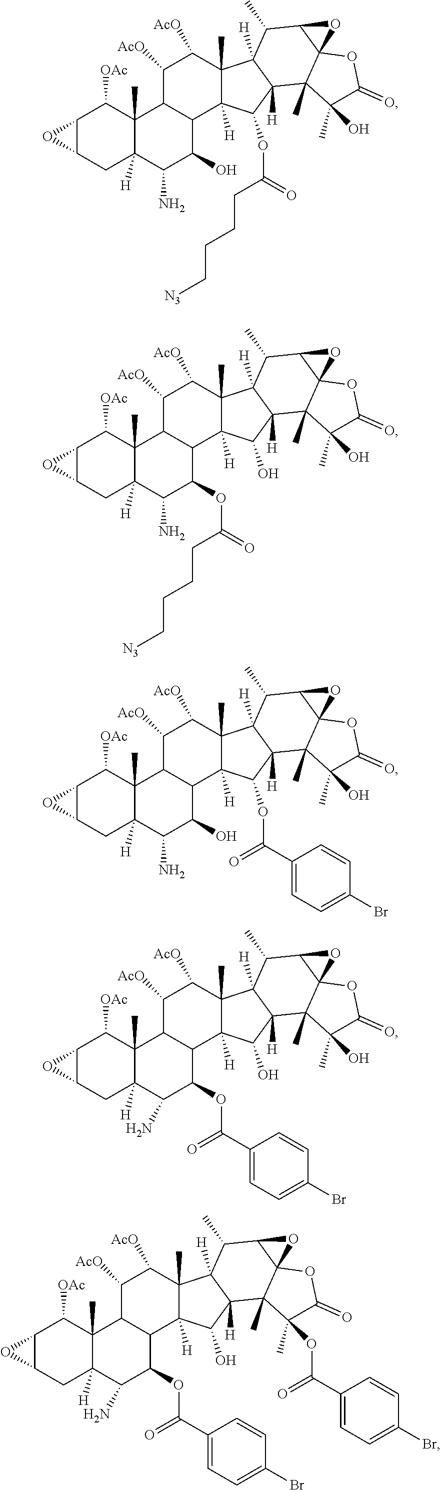

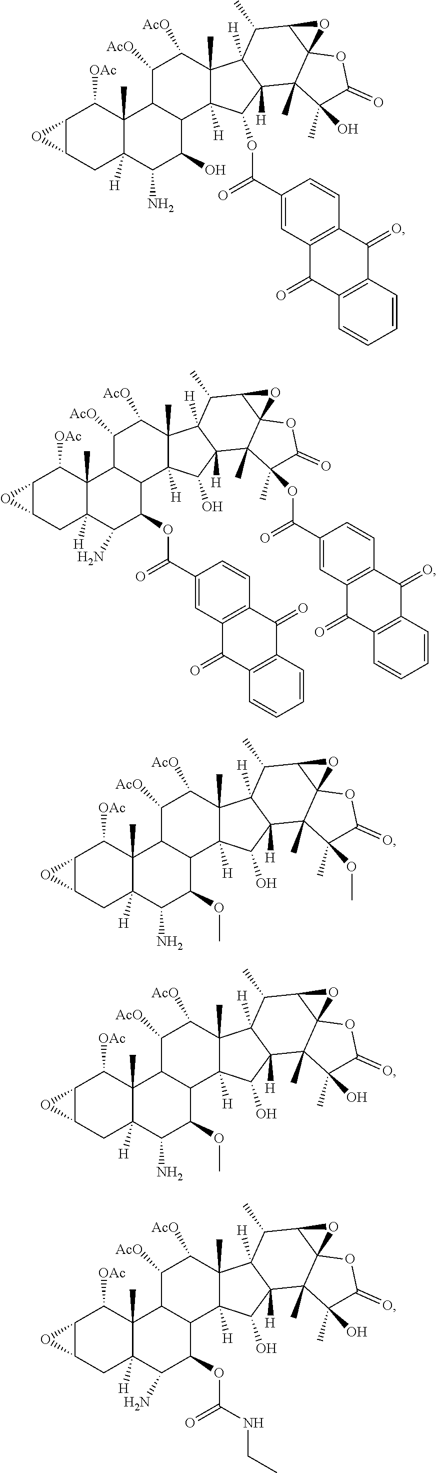

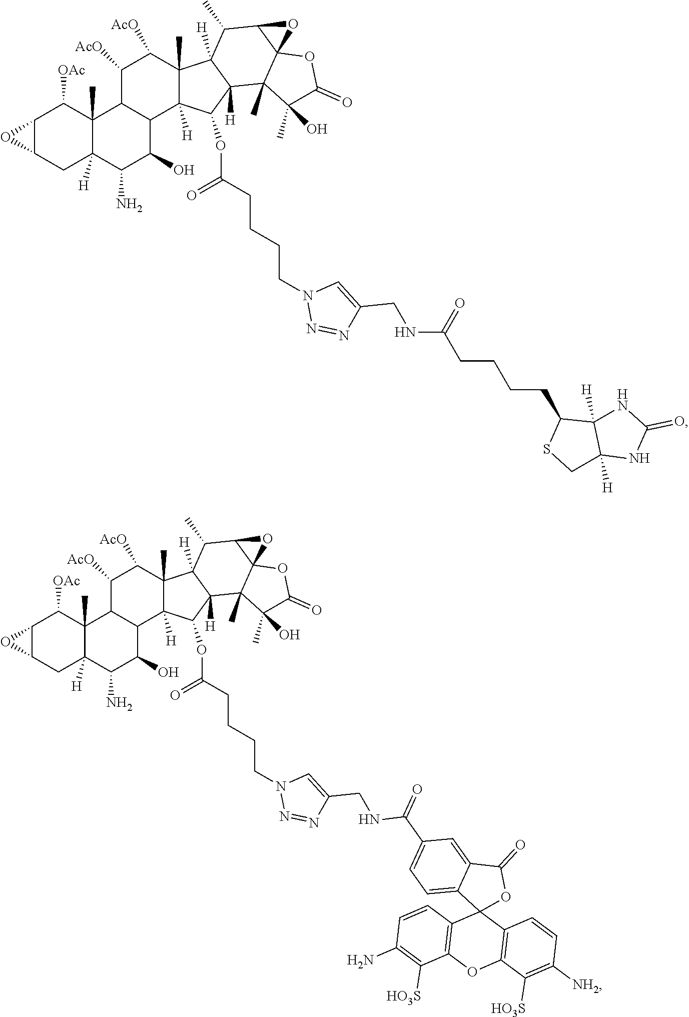

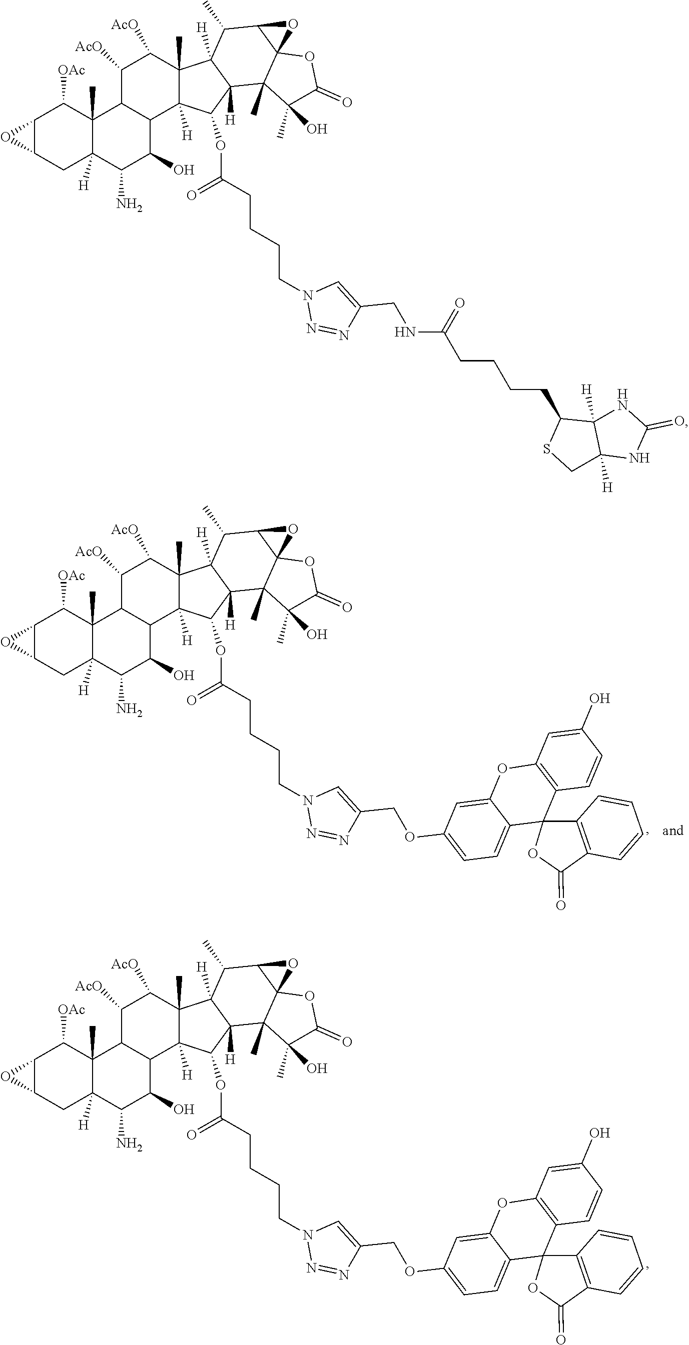

or a pharmaceutically acceptable salt thereof.

In a further aspect, the hyperproliferative disorder is a cardiovascular disease. Examples of cardiovascular disease include, but are not limited to, coronary heart disease, stroke, hypertensive heart disease, inflammatory heart disease, and rheumatic heart disease.

In a further aspect, the hyperproliferative disorder is a cancer. Examples of cancers include, but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

In various aspects, further examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma. In a still further aspect, the cancer is selected from chronic lymphocytic leukemia, small lymphocytic lymphoma, B-cell non-Hodgkin lymphoma, and large B-cell lymphoma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In one aspect, the cancer can be a cancer selected from cancers of the blood, brain, genitourinary tract, gastrointestinal tract, colon, rectum, breast, kidney, lymphatic system, stomach, lung, pancreas, and skin. In a further aspect, the cancer is selected from prostate cancer, glioblastoma multiforme, endometrial cancer, breast cancer, and colon cancer. In a further aspect, the cancer is selected from a cancer of the breast, ovary, prostate, head, neck, and kidney. In a still further aspect, the cancer is selected from cancers of the blood, brain, genitourinary tract, gastrointestinal tract, colon, rectum, breast, liver, kidney, lymphatic system, stomach, lung, pancreas, and skin. In a yet further aspect, the cancer is selected from a cancer of the lung and liver. In an even further aspect, the cancer is selected from a cancer of the breast, ovary, testes, and prostate. In a still further aspect, the cancer is a cancer of the breast. In a yet further aspect, the cancer is a cancer of the ovary. In an even further aspect, the cancer is a cancer of the prostate. In a still further aspect, the cancer is a cancer of the testes.

In a further aspect, the cancer is selected from a cancer of the breast, cervix, gastrointestinal tract, colorectal tract, brain, skin, prostate, ovary, thyroid, testes, genitourinary tract, pancreas, and endometrias. In a still further aspect, the cancer is a cancer of the breast. In yet a further aspect, the cancer of the breast is a hormone resistant cancer. In an even further aspect, the cancer of the breast is a hormone resistant cancer. In a still further aspect, the cancer is a cancer of the cervix. In yet a further aspect, the cancer is a cancer of the ovary. In an even further aspect, the cancer is a cancer of the endometrias. In a still further aspect, the cancer is a cancer of the genitourinary tract. In yet a further aspect, the cancer is a cancer of the colorectal tract. In an even further aspect, the cancer of the colorectal tract is a colorectal carcinoma. In a still further aspect, the cancer is a cancer of the gastrointestinal tract. In yet a further aspect, the cancer of the gastrointestinal tract is a gastrointestinal stromal tumor. In an even further aspect, the cancer is a cancer of the skin. In a still further aspect, the cancer of the skin is a melanoma. In yet a further aspect, the cancer is a cancer of the brain. In an even further aspect, the cancer of the brain is a glioma. In a still further aspect, the glioma is glioblastoma multiforme. In yet a further aspect, glioma is selected from is selected from an ependymoma, astrocytoma, oligodendroglioma, and oligoastrocytoma. In an even further aspect, the cancer of the brain is selected from acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, and hemangiopercytoma. In a still further aspect, the hematological cancer is selected from a leukemia, lymphoma, chronic myeloproliferative disorder, myelodysplastic syndrome, myeloproliferative neoplasm, and plasma cell neoplasm (myeloma). In yet a further aspect, the hematological cancer is leukemia. In an even further aspect, the leukemia is selected from acute leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, and chronic lymphocytic leukemia. In a still further aspect, the leukemia is acute lymphocytic leukemia. In yet a further aspect, the hematological cancer is lymphoma. In an even further aspect, the hematological cancer is myeloma. In a still further aspect, the myeloma is multiple myeloma.

In a further aspect, the carcinoma is selected from colon carcinoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, bile duct carcinoma, choriocarcinoma, seminoma, embryonal carcinoma, lung carcinoma, small cell lung carcinoma, bladder carcinoma, and epithelial carcinoma.

In a further aspect, the cancer is selected from breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma).

In a further aspect, the subject has been diagnosed with a need for treatment of a hyperproliferative disorder prior to the administering step.

In a further aspect, the subject is a mammal. In a still further aspect, the mammal is a human.

In a further aspect, the method further comprises the step of identifying a subject in need of treatment of a hyperproliferative disorder.

In a further aspect, the method further comprises the step of administering a therapeutically effective amount of at least one chemotherapeutic agent. Examples of chemotherapeutic agents include, but are not limited to, alkylating agents such as busulfan, cis-platin, mitomycin C, and carboplatin; antimitotic agents such as colchicine, vinblastine, paclitaxel (e.g., TAXOL®), and docetaxel; topoisomerase I inhibitors such as camptothecin and topotecan; topoisomerase II inhibitors such as doxorubicin and etoposide; RNA/DNA antimetabolites such as 5-azacytidine, 5-fluorouracil and methotrexate; DNA antimetabolites such as 5-fluoro-2′-deoxy-uridine, ara-C, hydroxyurea, gemcitabine, capecitabine and thioguanine; antibodies such as HERCEPTIN® and RITUXAN®, as well as other known chemotherapeutics such as photofrin, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen and alanosine.

In a further aspect, the at least one compound and the at least one agent are administered sequentially. In a still further aspect, the at least one compound and the at least one agent are administered simultaneously.

In a further aspect, the at least one compound and the at least one agent are co-formulated. In a still further aspect, the at least one compound and the at least one agent are co-packaged.

In a further aspect, the effective amount is a therapeutically effective amount. In a still further aspect, the effective amount is a prophylactically effective amount.

3. Additional Uses of Compounds

In one aspect, the invention relates to the use of a disclosed compound or a product of a disclosed method. In a further aspect, a use is as a probe. In a still further aspect, a use is as an ADC. In yet a further aspect, a use relates to the manufacture of a medicament for the treatment of a hyperproliferative disorder in a mammal. In a still further aspect, a use relates to the manufacture of a medicament for the treatment of cancer in a mammal. In yet a further aspect, the use relates to the manufacture of a medicament for the treatment of cardiovascular disease in a mammal.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of a disorder in a mammal. In one aspect, the use is characterized in that the mammal is a human. In one aspect, the use is characterized in that the disorder is a hyperproliferative disorder. In one aspect, the use is characterized in that the disorder is a cancer or a cardiovascular disease.

In a further aspect, the use relates to the manufacture of a medicament for the treatment of a hyperproliferative disorder in a mammal.

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of a hyperproliferative disorder such as, for example, cancer or cardiovascular disease, in a mammal. In a further aspect, the cancer is selected from multiple myeloma and hematologic malignancy.

4. Manufacture of a Medicament

In one aspect, the invention relates to a method for the manufacture of a medicament for treating a hyperproliferative disorder in a mammal, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the treatment of a hyperproliferative disorder such as, for example, cancer or cardiovascular disease. The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

5. Kits

In one aspect, disclosed are kits comprising at least one disclosed compound and one or more of: (a) at least one agent associated with the treatment of a hyperproliferative disorder; (b) instructions for administering the compound in connection with treating a hyperproliferative disorder; and (c) instructions for treating a hyperproliferative disorder.

In a further aspect, the hyperproliferative disorder is a cancer. Examples of cancers for which the compounds and compositions can be useful in treating, include, but are not limited to carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

In various aspects, further examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas

In a further aspect, the agent associated with the treatment of a hyperproliferative disorder is a chemotherapeutic agent. Examples of chemotherapeutic agents include, but are not limited to, alkylating agents such as busulfan, cis-platin, mitomycin C, and carboplatin; antimitotic agents such as colchicine, vinblastine, paclitaxel (e.g., TAXOL®), and docetaxel; topoisomerase I inhibitors such as camptothecin and topotecan; topoisomerase II inhibitors such as doxorubicin and etoposide; RNA/DNA antimetabolites such as 5-azacytidine, 5-fluorouracil and methotrexate; DNA antimetabolites such as 5-fluoro-2′-deoxy-uridine, ara-C, hydroxyurea, gemcitabine, capecitabine and thioguanine; antibodies such as HERCEPTIN® and RITUXAN®, as well as other known chemotherapeutics such as photofrin, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen and alanosine. In a further aspect, the chemotherapeutic agent is selected from an alkylating agent, an antimetabolite agent, an antineoplastic antibiotic agent, a mitotic inhibitor agent, and a mTor inhibitor agent.

In a further aspect, the antineoplastic antibiotic agent is selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In a further aspect, the antimetabolite agent is selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In a further aspect, the alkylating agent is selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof.

In a further aspect, the mitotic inhibitor agent is selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof.

In a further aspect, the mTor inhibitor agent is selected from everolimus, siroliumus, and temsirolimus, or a pharmaceutically acceptable salt, hydrate, solvate, or polymorph thereof.

In a further aspect, the at least one compound and the at least one agent are co-formulated. In a further aspect, the at least one compound and the at least one agent are co-packaged.

In a further aspect, the compound and the agent are administered sequentially. In a still further aspect, the compound and the agent are administered simultaneously.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. Examples

Many successful therapeutics, including aspirin, β-lactam antibiotics, esomeprazole (Nexium), and clopidogrel (Plavix) (Singh, et al. (2011) Nat. Rev. Drug. Discov. 10, 307-317; Robertson, J. G. (2005) Biochemistry 44, 5561-5571) bind covalently to their drug targets. However, the irreversible nature of their binding prompts safety concerns due to potential off-target reactivity and unanticipated side effects. Therefore, one of the most critical steps in the covalent drug discovery process is the effective evaluation of their target specificity and assessment of useful de-risking strategies (Bauer, R. A. (2015) Drug. Discov. Today 20, 1061-1073; Johnson, et al. (2010) Future Med. Chem. 2, 949-964). The development of modern ‘targeted covalent inhibitors’ (TCIs) (Baillie, T. A. (2016) Angew. Chem. Int. Ed. Engl. 55, 13408-13421; Lonsdale, et al. (2018) Chem. Soc. Rev. 47, 3816-3830) has led to significant progress including the successful launch of several preclinical and clinical studies for covalent EGFR inhibitors, such as the FDA approved afatinib (Giltrif) and osimertinib (Tagrisso), which exhibited promising therapeutic effects against resistant cancer models expressing EGFR mutations (Irie, H. et al. (2019) Mol. Cancer Ther. 18, 733-742; Murakami, H. et al. (2017) Ann. Oncol. 28; Ito, K. et al. (2019) Mol. Cancer Ther. 18, 920-928). The systematic studies of TCIs have also revealed that the safety of covalent drugs needs to be evaluated on a case-by-case basis and the complexity of the covalent systems often urge innovative approaches (Ziegler, et al. (2013) Angew. Chem. Int. Edit. 52, 2744-2792; Bottcher, et al. (2010) Angew. Chem. Int. Edit. 49, 2680-2698; Wright, et al. (2016) Nat. Prod. Rep. 33, 681-708) as necessary complements to conventional preclinical and clinical studies.

The taxane class of microtubule stabilizers is a mainstay in the clinical treatment of solid tumors even in the era of targeted therapy and immunotherapy (Broggini-Tenzer, A. et al. (2015) J. Natl. Cancer. Inst. 107, dju504; Jhaveri, K. et al. (2017) Breast Cancer Res. 19, 89; Rohena, et al. (2014) Nat. Prod. Rep. 31, 335-355; Schmid, P. et al. (2018) N. Engl. J. Med. 379, 2108-2121). However, a major limitation of the taxanes is acquired drug resistance. The taccalonolides are a class of microtubule stabilizers that covalently bind β-tubulin (Risinger, A. L. et al. (2013) Cancer Res. 73, 6780-6792; Wang, Y. et al. (2017) Nat. Commun. 8, 15787) and effectively circumvent clinically relevant models of resistance to taxanes both in vitro and in vivo (Tinley, T. L. et al. (2003) Cancer Res. 63, 3211-3220; Risinger, A. L. et al. (2008) Cancer Res. 68, 8881-8888; Ola, A. R. B. et al. (2018) J. Nat. Prod. 81, 579-593). Despite their promising therapeutic potential, the covalent nature of taccalonolide binding has hampered the ability to perform detailed binding studies using conventional approaches (de Jong, L. A. A., et al. (2005) J. Chromatogr. B 829, 1-25; Pollard, T. D. (2010) Mol. Biol. Cell 21, 4061-4067). Consequently, it urged the development of a functional and rigorous activity-based approach to elucidate the target specificity and drug-target interactions of the taccalonolides. Here, the synthesis and optimization of a fluorogenic taccalonolide probe, Flu-tacca-7 (11) is described. This stable, cell-permeable probe is used for activity-based protein profiling (ABPP) in human cancer cell lines to confirm the specificity of covalent binding of the taccalonolides to β-tubulin and evaluate key β-tubulin residues and taccalonolide moieties that mediate taccalonolide-tubulin binding. Flu-tacca-7 (11) represents a class of irreversible microtubule labeling probes that are superior to commercially available options, providing a valuable tool for cellular evaluations of this important drug target.

The following preparations and examples are given to enable those skilled in the art to more clearly understand and to practice the present invention. They should not be considered as limiting the scope of the invention, but merely as being illustrative and representative.

### 1. General Experimental Methods

Optical rotations were measured on a Rudolph Research Autopol III automatic polarimeter. NMR data were obtained on Varian VNMR spectrometers (400 and 500 MHz for ¹H, 100 and 125 MHz for ¹³C) with broad band and triple resonance probes. Preparative HPLC separations were performed on a Shimadzu system using a SCL-10A VP controller and a Gemini 5 μm C18 or a Kinetex F5 5 μm C18 column (110 Å, 250×21.2 mm) with flow rate of 10 mL/min. Semi-preparative HPLC separations were performed on a Waters 1525 system using a 2998 PDA detector and Gemini 5 μm C18 or a Kinetex F5 5 μm C18 column (110 A, 250×10.0 mm) with flow rate of 4 mL/min. All solvents were of ACS grade or better. HRESIMS (high-resolution electrospray ionization mass spectrometry) data were collected on an Agilent 6538 high-mass-resolution QTOF mass spectrometer. X-ray Intensity data were collected using a diffractometer with a Bruker APEX ccd area detector and graphite-monochromated Mo Kα radiation (λ=0.71073 Å).

### 2. Synthesis of Taccalonolide Analogs

a. Compound Nos. 152E and 152D

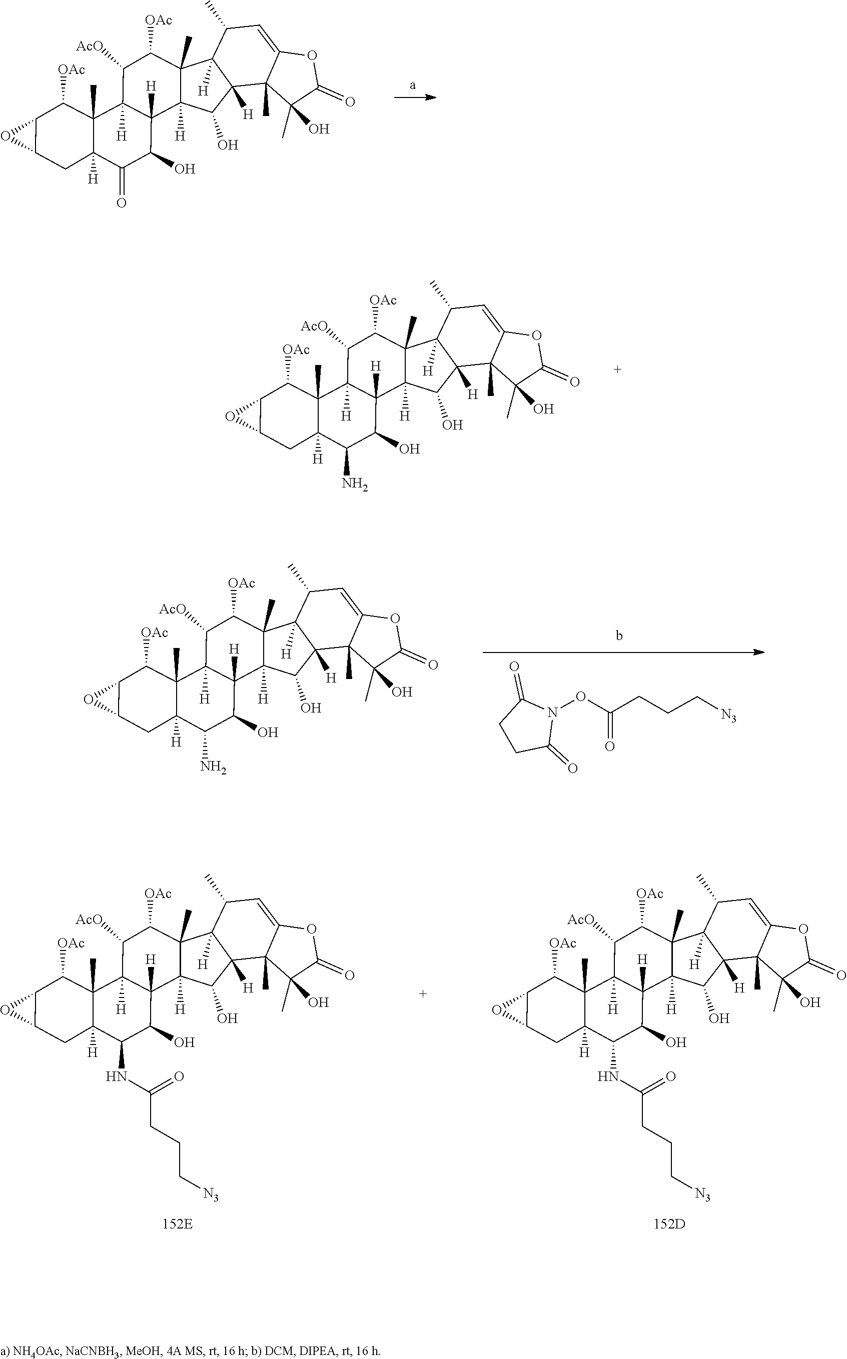

b. Compound No. 154B

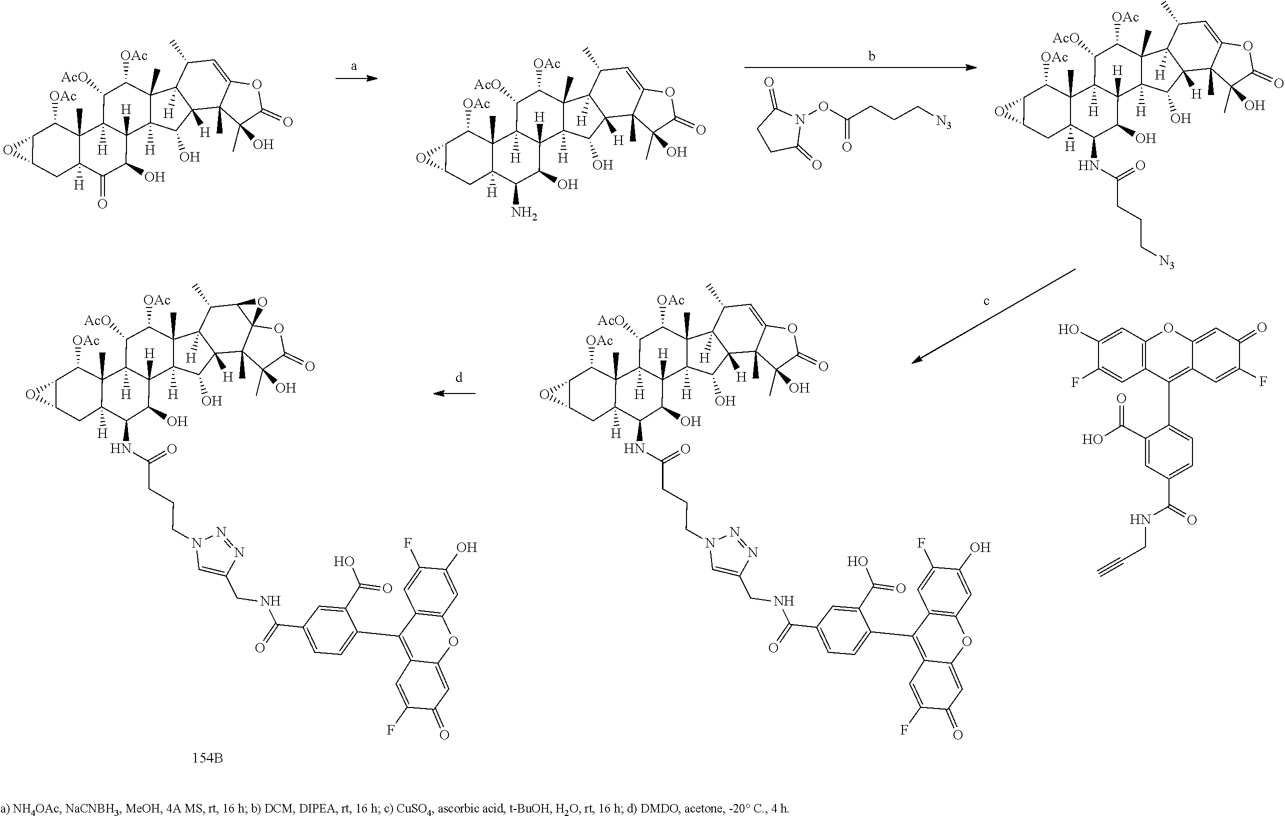

c. Compound No. 154C

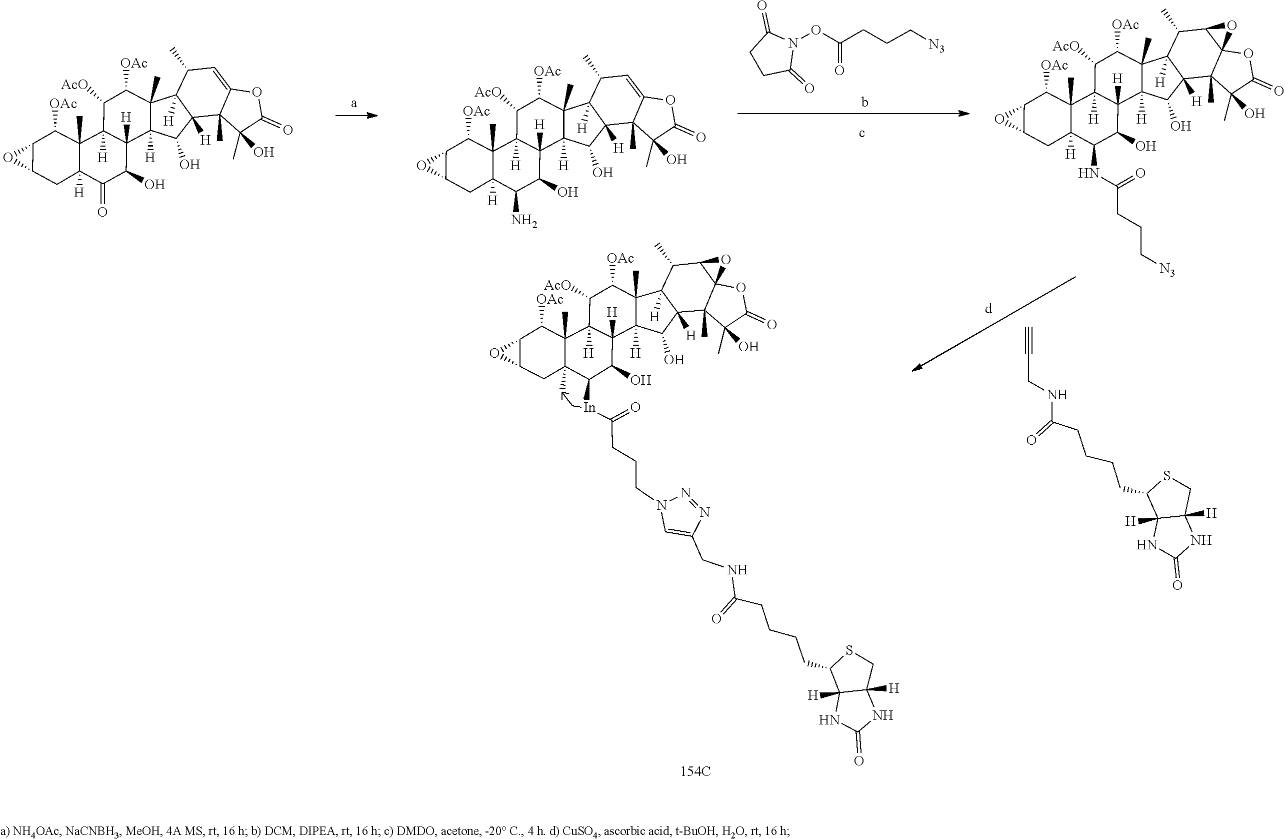

d. Compound No. 154J

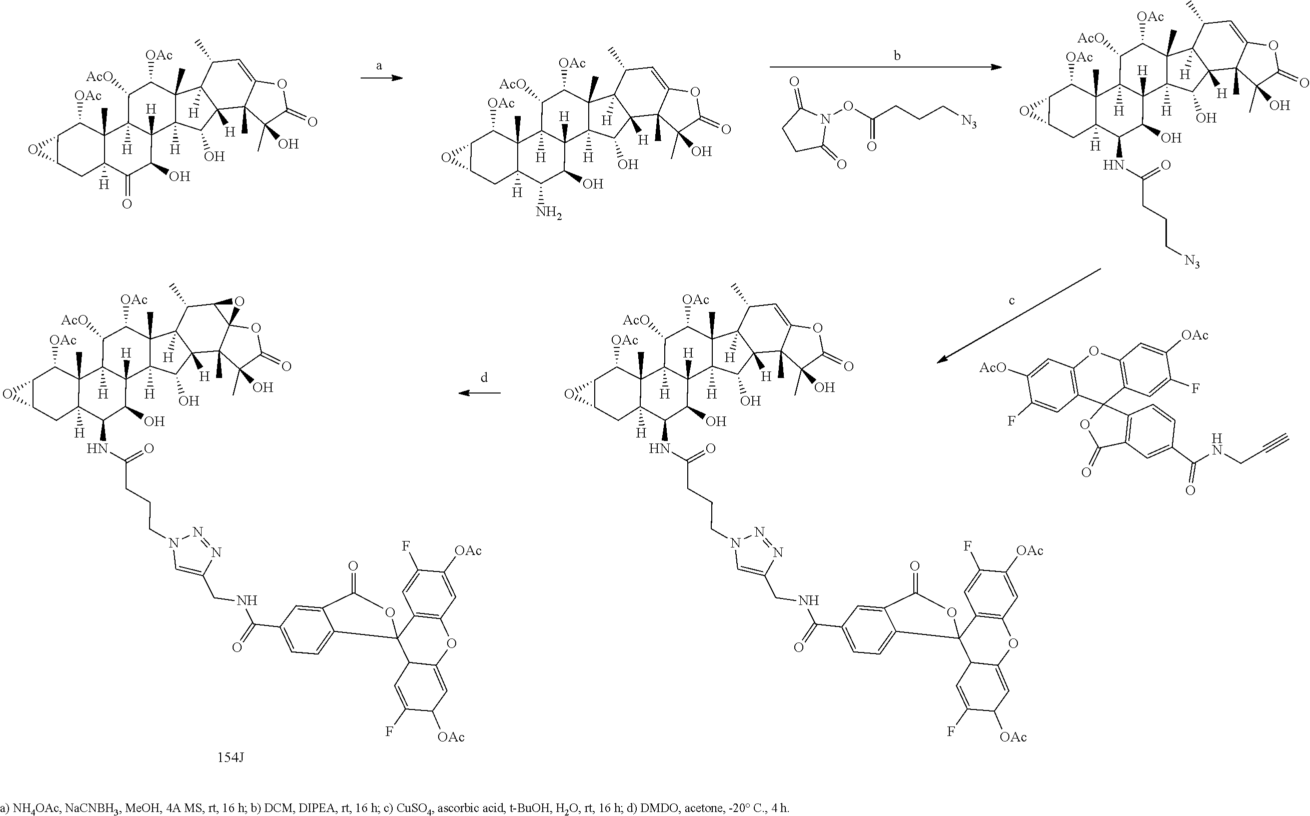

e. Compound No. 155F

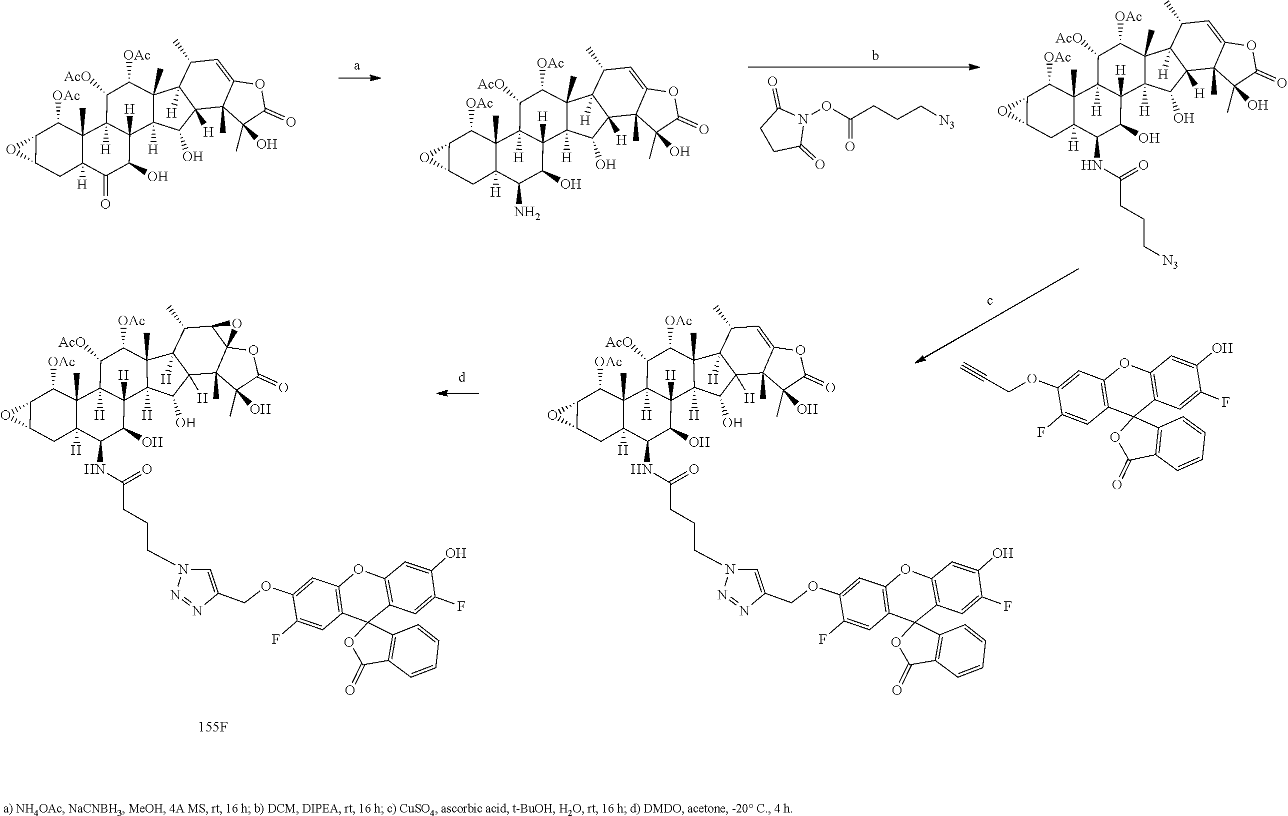

f. Compound No. 155G

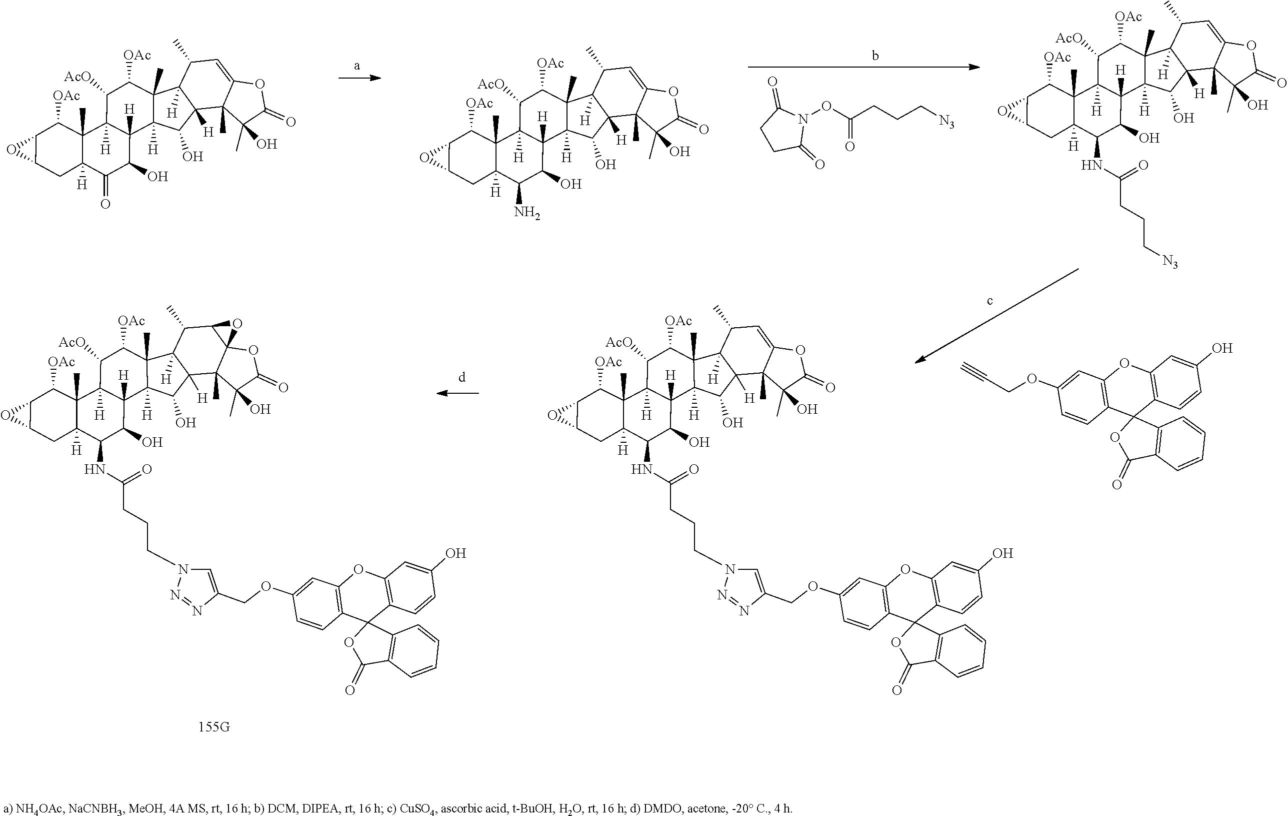

g. Compound No. 158E

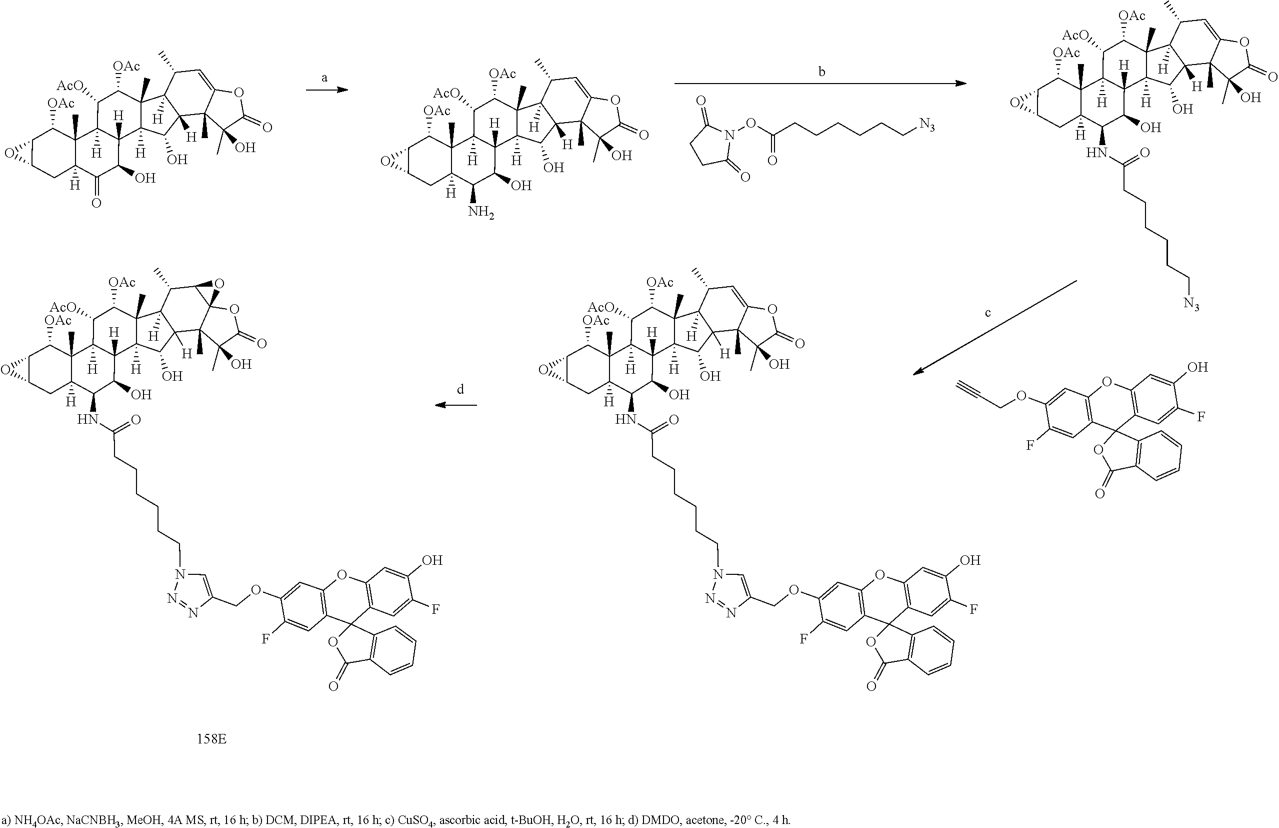

h. Compound No. 158F

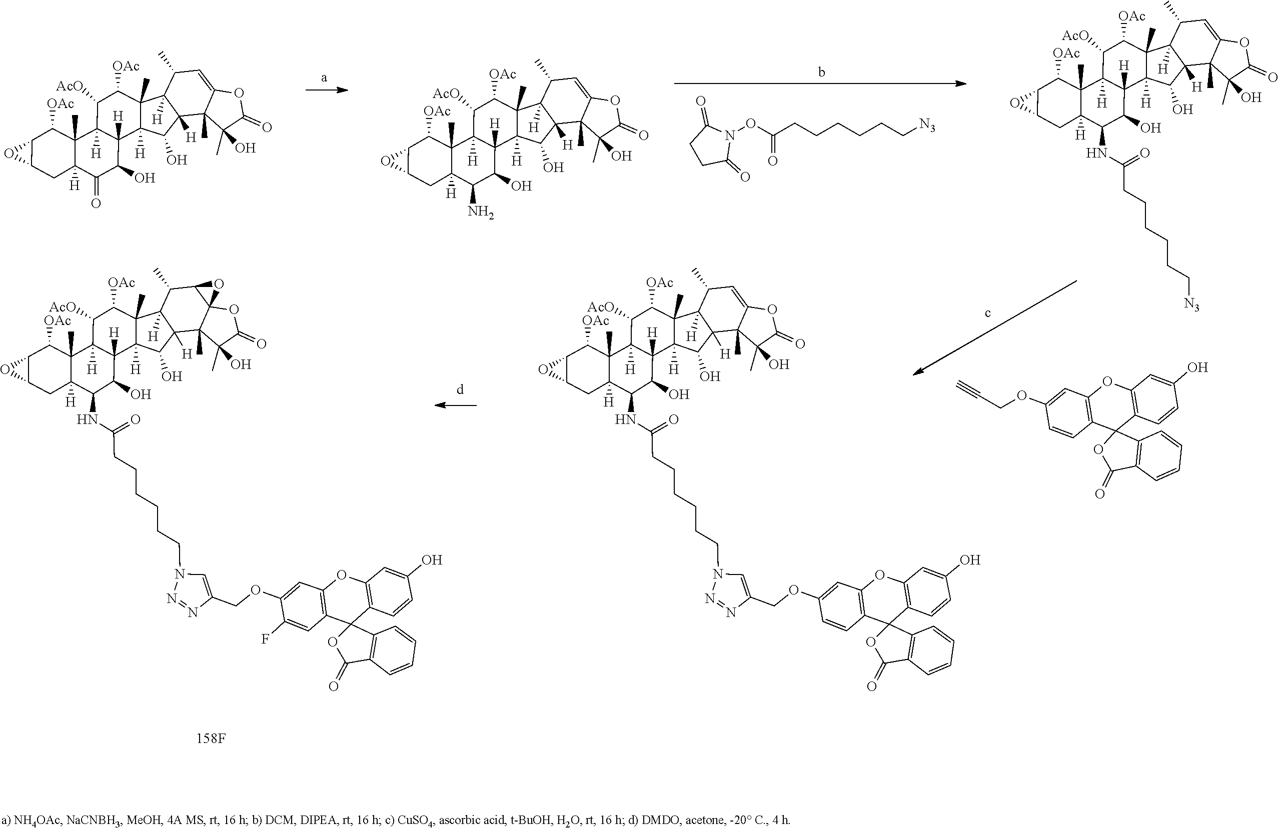

i. Compound no. 162C

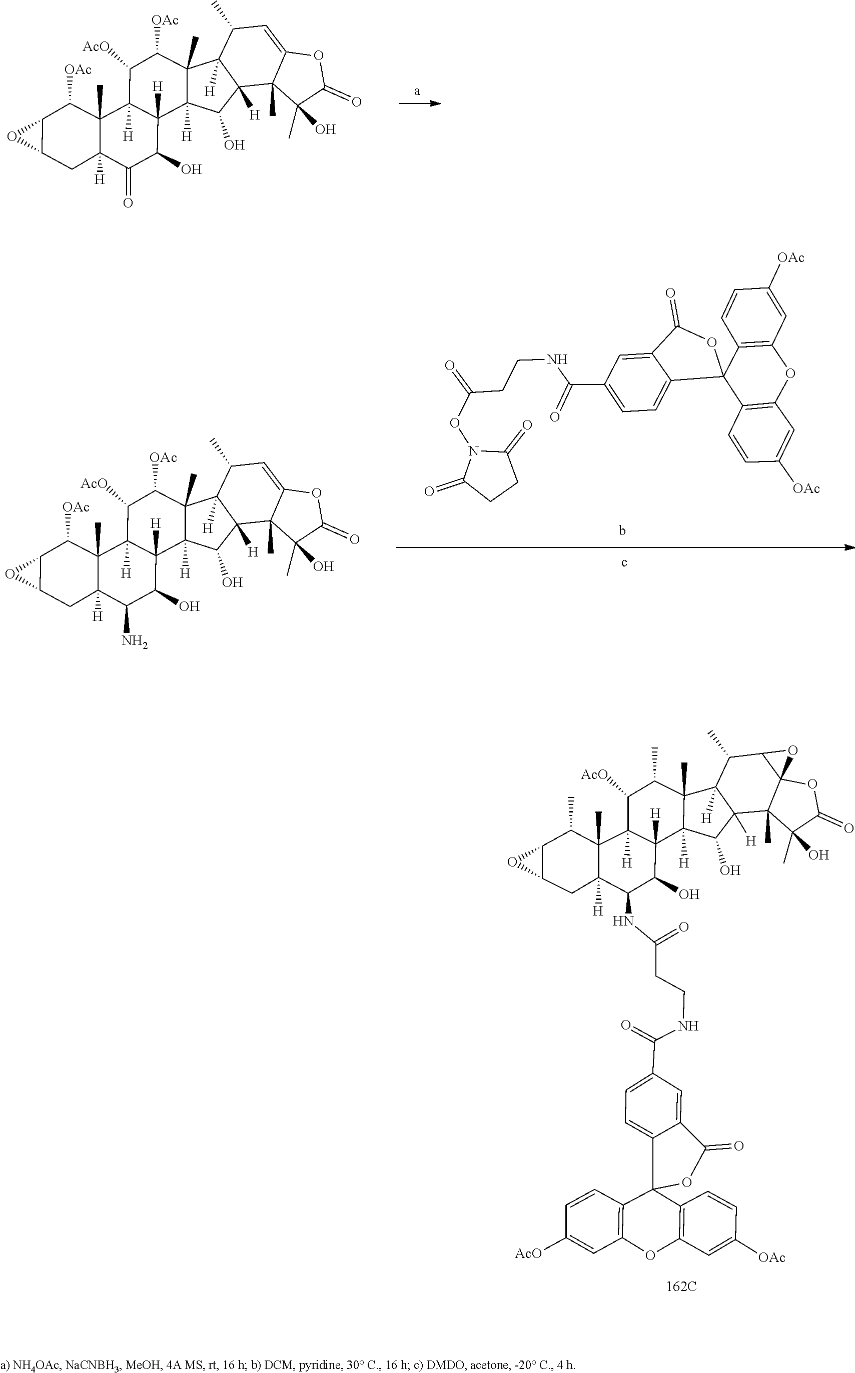

j. Compound No. 163D

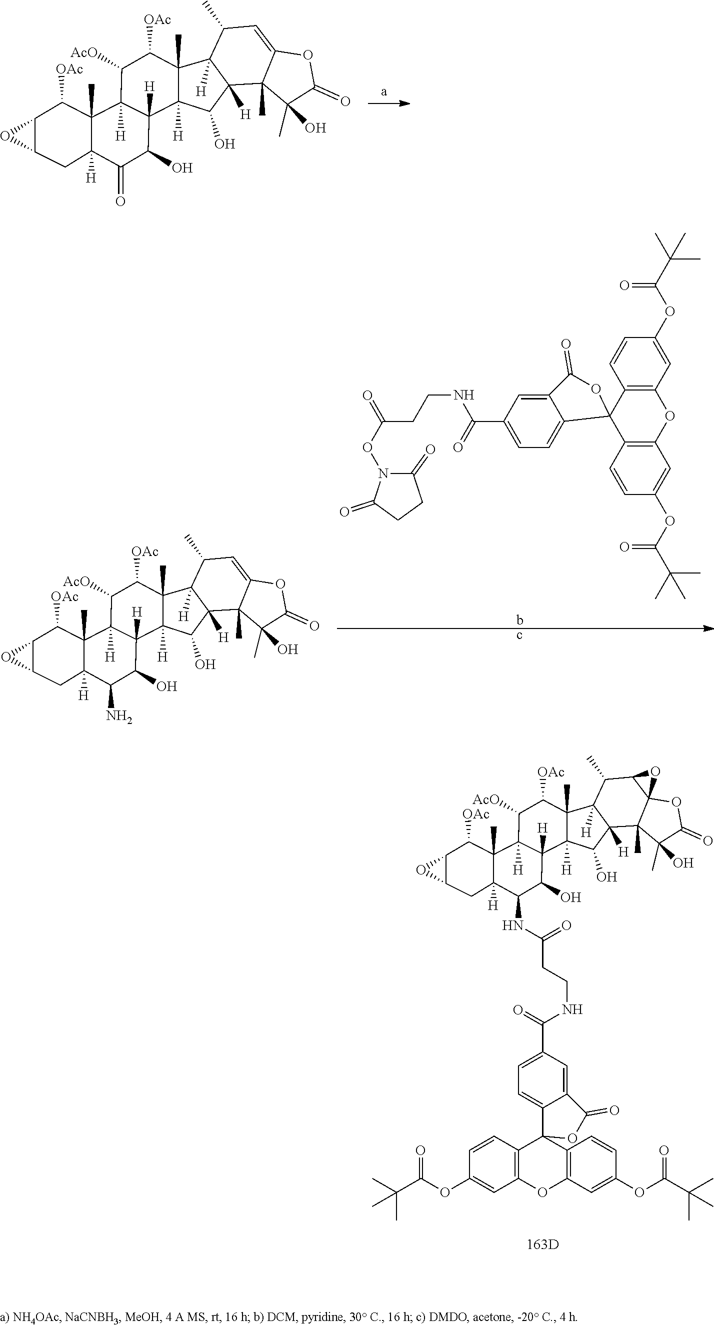

k. Compound No. 163E

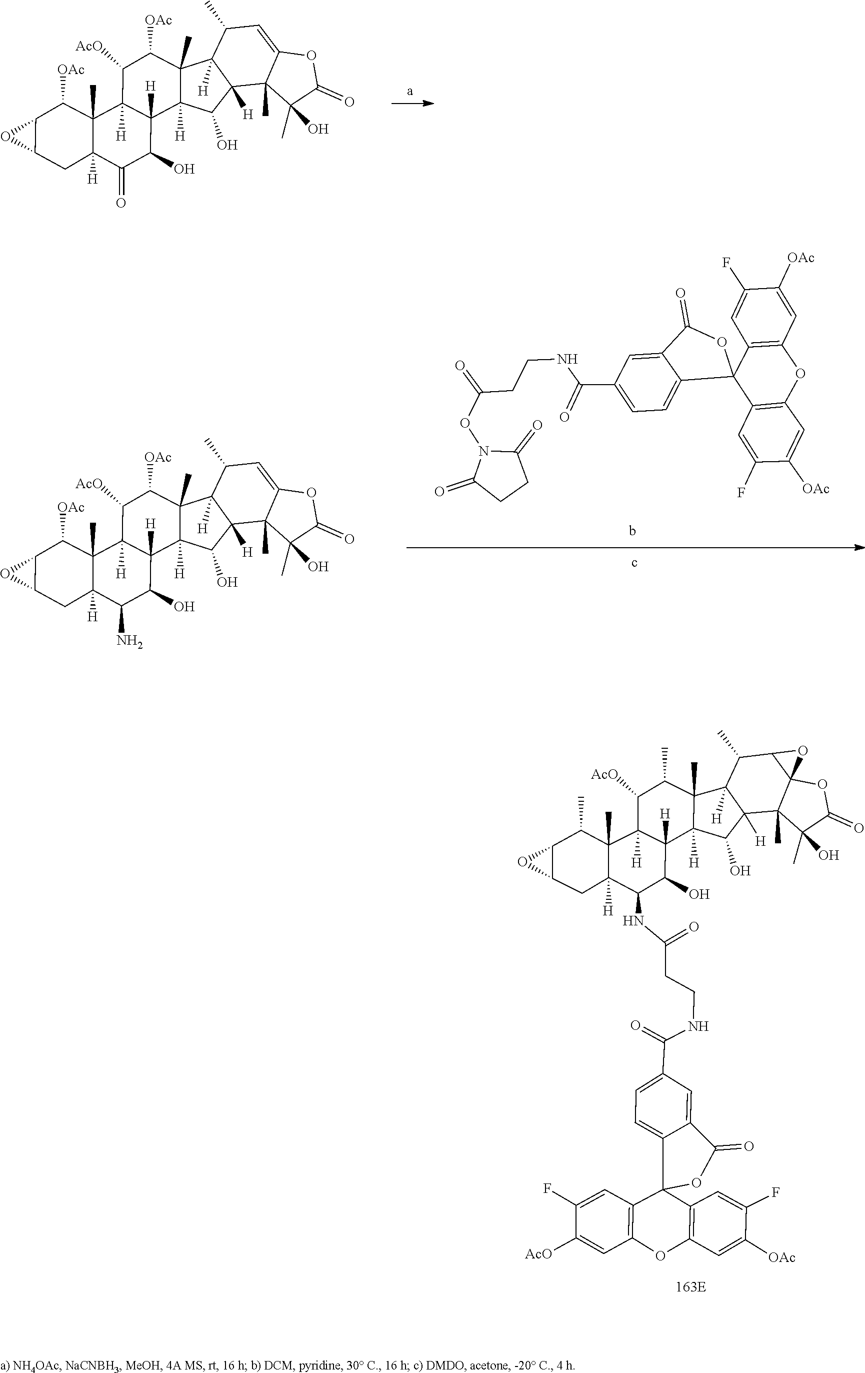

l. Compounds No. 164A and 164B

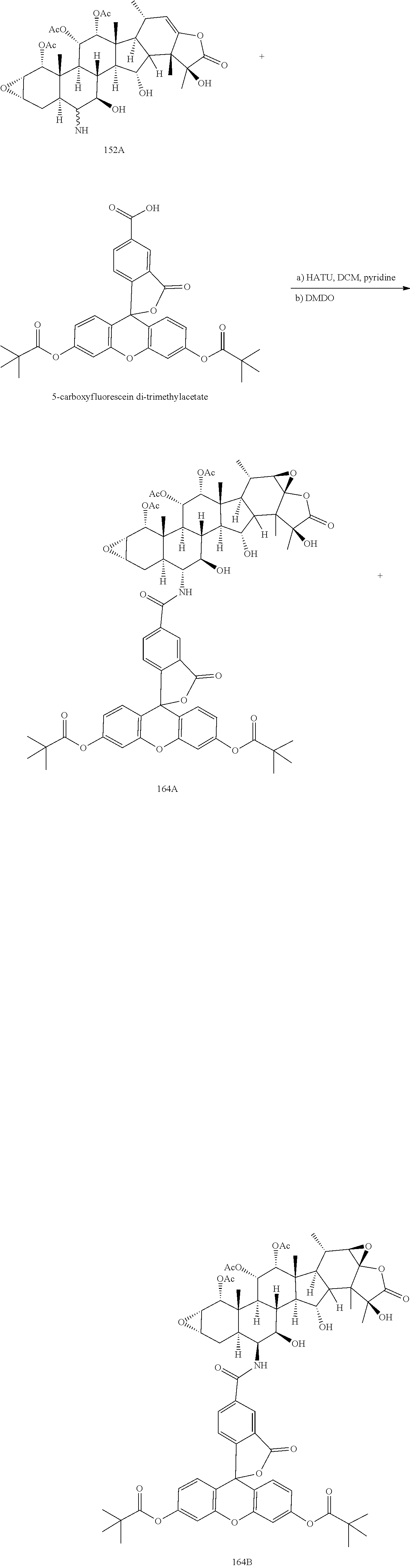

152A is a mixture of two 6-amino epimers resulted from the reductive amination of taccalonolide B. A sample of 152A (1 eq.) was stirred with 5-carboxyfluorescein di-trimethylacetate (1.5 eq.) and HATU (1 eq.) in a 1:1 mixture of DCM/pyridine at room temperature overnight. The solvents were removed in vacuo and the residue was purified by prep-HPLC using a C-18 column to yield compounds 164A (˜9% yield) and 164B (˜27% yield). Compound 164A: white powder; ¹H NMR (500 MHz, methanol-d₄) δ 8.51 (1H, s), 8.24 (1H, d, J=8.0 Hz), 7.37 (1H, d, J=8.0 Hz), 7.16 (2H, br s), 6.85-6.91 (4H, m), 5.33 (1H, dd, J=2.9, 12.0 Hz), 5.17 (1H, d, J=2.8 Hz), 4.73 (1H, d, J=5.3 Hz), 4.35 (1H, t, J=8.7 Hz), 4.02 (1H, t, J=11.1 Hz), 3.45 (1H, t, J=4.8 Hz), 4.39 (1H, t, J=10.0 Hz), 3.32 (1H, m), 3.30 (1H, m), 2.30 (1H, t, J=11.2 Hz), 2.23 (1H, dd, J=4.6, 15.4 Hz), 2.11 (3H, s), 2.09 (3H, s), 2.09-2.11 (2H, m), 1.97 (1H, dd, J=8.8, 10.5 Hz), 1.91 (3H, s), 1.87-1.93 (2H, m), 1.79 (1H, d, J=15.4 Hz), 1.74 (3H, s), 1.56 (1H, m), 1.35 (18H, s), 1.26 (3H, s), 1.02 (3H, s), 1.00 (3H, s, J=7.3 Hz), 0.90 (3H, s); Compound 164B: white powder; ¹H NMR (500 MHz, methanol-d₄) δ 8.36 (1H, s), 8.10 (1H, dd, J=1.6, 8.0 Hz), 7.35 (1H, d, J=8.0 Hz), 7.17 (2H, s), 6.85-6.91 (4H, m), 5.30 (1H, dd, J=2.9, 11.2 Hz), 5.16 (1H, d, J=2.9 Hz), 4.69 (1H, d, J=5.5 Hz), 4.60 (1H, m), 4.27 (1H, t, J=8.9 Hz), 3.78 (1H, dd, J=4.8, 10.0 Hz), 3.40 (1H, dd, J=3.8, 5.6 Hz), 3.33 (1H, m), 3.30 (1H, m), 2.29-2.37 (1H, m), 2.12 (3H, s), 2.11 (3H, s), 2.05-2.12 (5H, m), 1.97 (1H, t, J=9.3 Hz), 1.91 (3H, s), 1.74 (3H, s), 1.55 (1H, m), 1.35 (18H, s), 1.24 (3H, s), 1.08 (3H, s), 1.00 (3H, s, J=7.3 Hz), 0.91 (3H, s).

m. Compound No. 164E

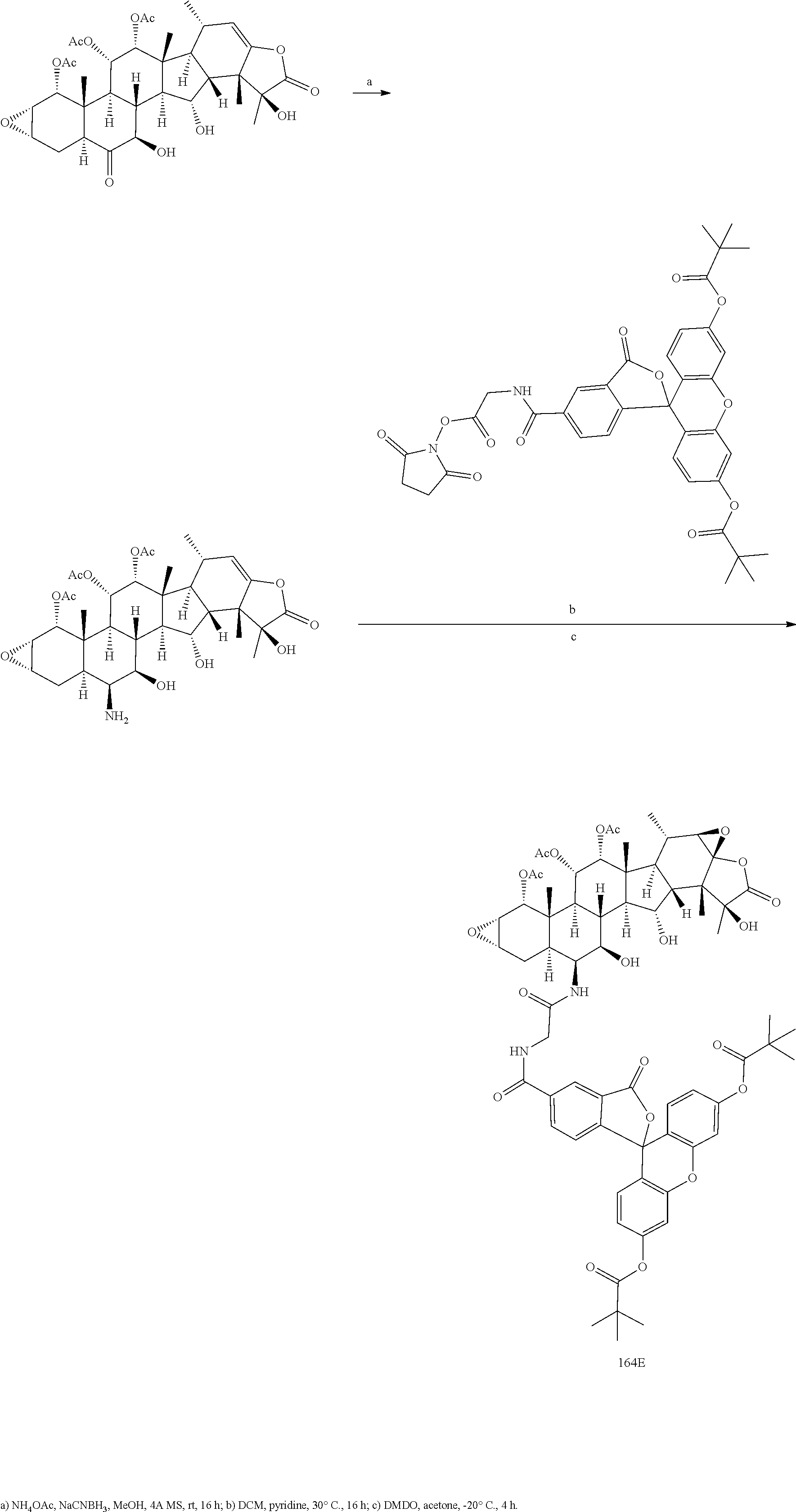

n. Compound No. 167C

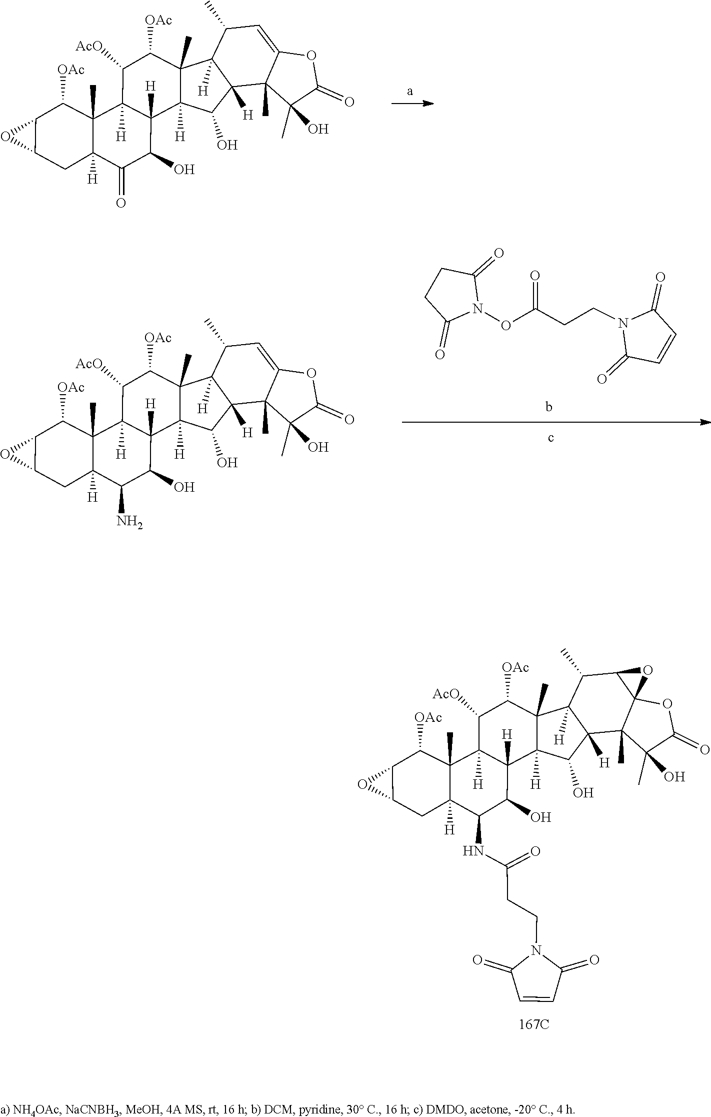

o. Conjugated Compound No. 167C

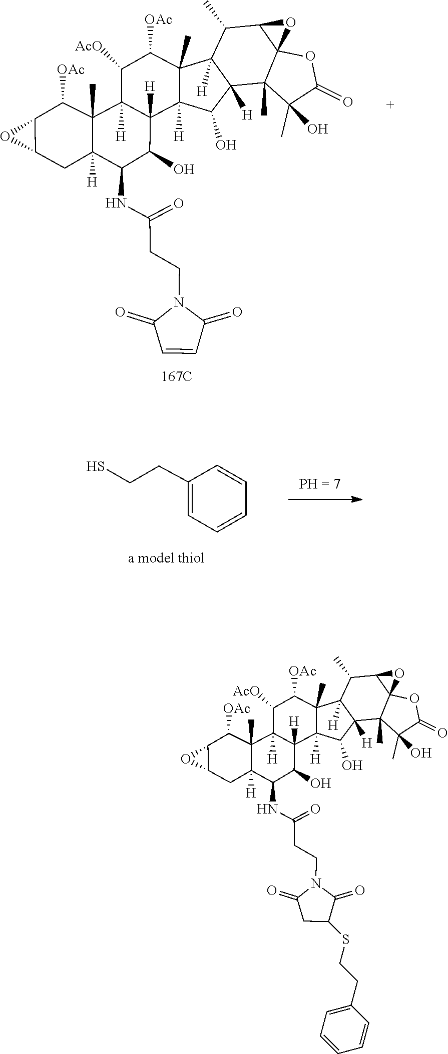

p. Compound No. 171B_TFA

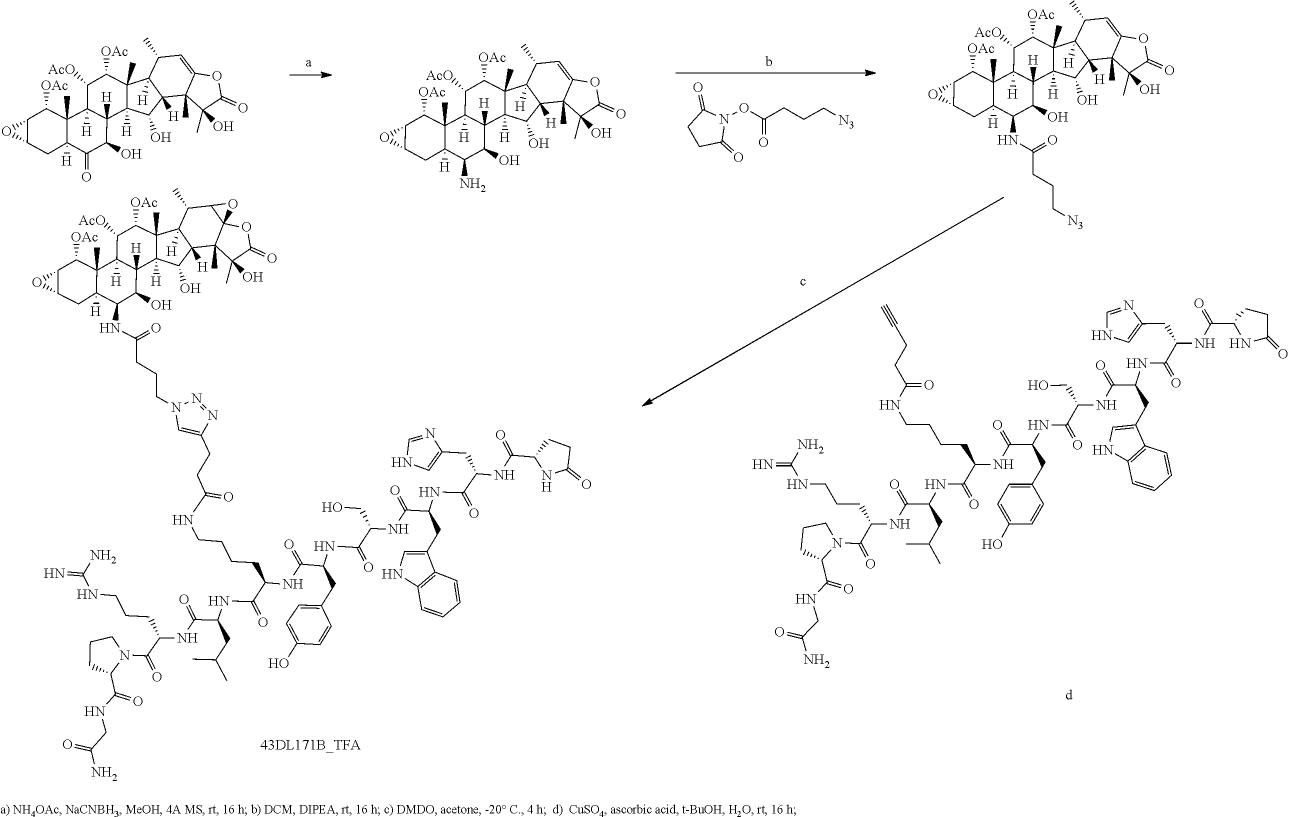

### 3. Reductive Amination of Taccalonolide B (13)

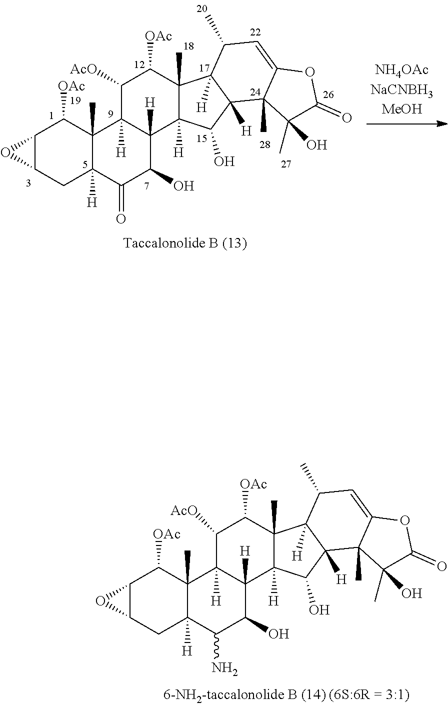

Taccalonolide B (13, 50 mg, 1 equiv) was mixed with ammonium acetate (165 mg, 30 equiv), sodium cyanoborohydride (45 mg, 10 equiv), and 4 Å molecular sieves (500 mg) in anhydrous methanol (MeOH) (4 mL). The reactant mixture was stirred at 35° C. overnight and the solvent was removed in vacuo. The residue was purified by preparative HPLC using a Luna 5 μm C1S column [isocratic, 40% acetonitrile (MeCN) in 0.1% trifluoroacetic acid (TFA)] to yield the TFA salt TFA-14 (45 mg) which was identified as a 3:1 mixture of the 6S and 6R epimers by analysis of 1D and 2D NMR data. The TFA salt was then stirred with the AMBERLYST™ A21 resin (450 mg) in methanol (4 mL) for 2 h at room temperature. The resin was filtered and washed with methanol (4 mL×2). The combined methanol filtrates were evaporated in vacuo to yield the free amine 6-NH₂-taccalonolide B (14) (33 mg).

6-NH₂-taccalonolide B (14): colorless solid; ¹H and ¹³C NMR data of TFA-14, see Table i; HRESIMS (m/z): [M+H]⁺ calcd. for C₃₄H₄₈NO₁₂, 662.3177; found 662.3177.

### 4. Synthesis of 5-carboxyfluorescein Dipivalate (16)

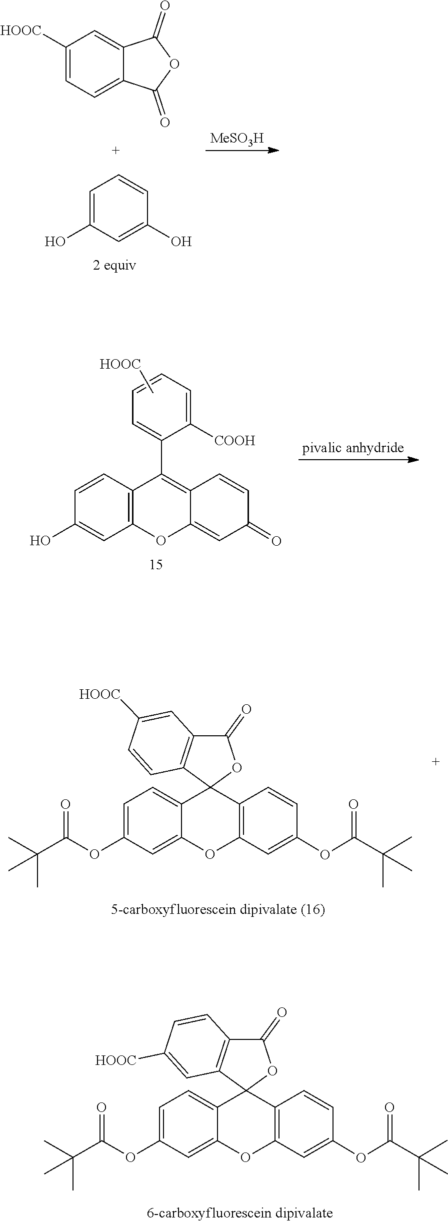

Trimellitic anhydride (436 mg, 1 equiv) and resorcinol (500 mg, 2 equiv) were stirred in methanesulfonic acid (12 mL) at 80° C. for 24 h. The resulted mixture was poured into ice water (H₂O). The precipitate was collected and washed with water to yield the crude 5(6)-carboxyfluorescein (15) (820 mg).

5(6)-carboxyfluorescein (15): yellow powder; ¹H NMR (500 MHz, methanol-d₄): δ 8.59 (d, J=1.5 Hz, 1H), 8.37 (dd, J=8.0, 1.5 Hz, 1H), 8.31 (dd, J=8.0, 1.5 Hz, 1H), 8.09 (d, J=8.0 Hz, 1H), 7.74 (s, 1H), 7.30 (d, J=8.0 Hz, 1H), 6.70-6.72 (d, J=9.7 Hz, 4H), 6.60-6.42 (br s, 4H), 6.53-6.57 (m, 4H). See also (Ueno, et al. (2004) Synthesis-Stuttgart, 2591-2593, doi:10.1055/s-2004-829194.

5(6)-carboxyfluorescein (15) (490 mg) was stirred in trimethylacetic anhydride (8 mL, 30 equiv) at 110° C. for 3 h. The resulted mixture was then stirred in the mixed solution of water (15 mL) and THE (30 mL) at room temperature for 2 days. The solvents were removed in vacuo and the residue was purified by preparative HPLC using a Luna 5 μm C18 column (isocratic, 80% MeCN in 0.1% TFA) to yield 5-carboxyfluorescein dipivalate (16) (221 mg) and 6-carboxyfluorescein dipivalate (198 mg).

5-carboxyfluorescein dipivalate (16): yellow powder; ¹H NMR (400 MHz, methanol-d₄): δ 8.56 (s, 1H), 8.23 (d, J=8.0 Hz, 1H), 7.12 (dd, J=8.0, 1.6 Hz, 1H), 7.05 (br s, 2H), 6.75-6.80 (m, 4H), 1.28 (s, 18H); ¹³C NMR (100 MHz, methanol-d₄): δ 177.7, 169.6, 167.6, 157.6, 154.1, 152.6, 137.6, 134.4, 130.0, 127.6, 127.5, 125.3, 119.1, 116.8, 111.4, 83.1, 40.0, 27.4. See also Oberg, et al. (2003) Bioconjugate Chem 14, 1289-1297, doi:10.1021/bc034130j.

### 5. Synthesis of 5-carboxyfluorescein Diacetate (17)

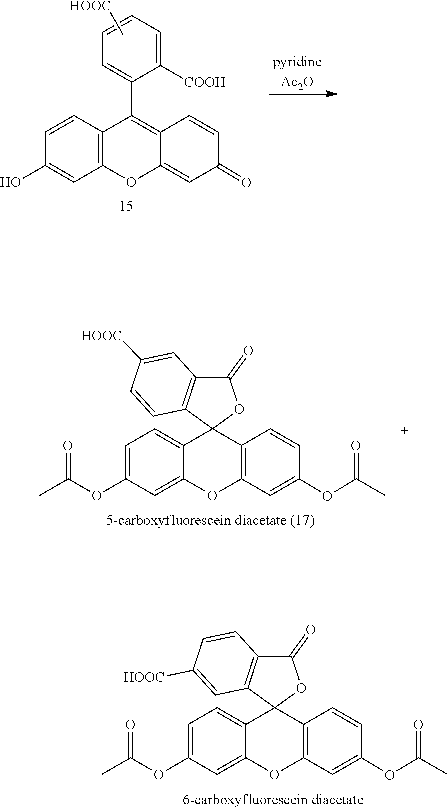

5(6)-carboxyfluorescein (15) (320 mg) and pyridine (260 μL) were stirred in acetic anhydride (5 mL) at 80° C. for 5 min. The solvents were removed in vacuo and the residue was purified by preparative HPLC using a Luna 5 μm C18 column (isocratic, 50% MeCN in 0.1% formic acid) to yield 5-carboxyfluorescein diacetate (117) (161 mg) and 6-carboxyfluorescein diacetate (152 mg).

5-carboxyfluorescein diacetate (17): yellow powder; ¹H NMR (400 MHz, methanol-d₄): δ 8.61 (s, 1H), 8.37 (d, J=8.0 Hz, 1H), 7.32 (d, J=8.0 Hz, 1H), 7.18 (br s, 2H), 6.88 (br s, 4H), 2.28 (s, 6H).

### 6. Synthesis of 5(6)-carboxy-Oregon Green 488 (19)

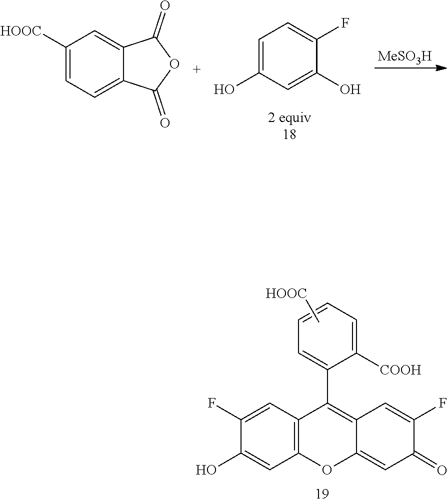

Trimellitic anhydride (375 mg, 1 equiv) and 4-fluororesorcinol (18) (500 mg, 2 equiv) were stirred in methanesulfonic acid (12 mL) at 80° C. for 24 h. The resulted mixture was poured into ice water. The precipitate was collected and washed with water to yield the crude 5(6)-carboxy-Oregon Green 488 (19) (930 mg).

5(6)-carboxy-Oregon Green 488 (19): yellow powder; ¹H NMR (500 MHz, methanol-d₄): δ 8.61 (d, J=1.5 Hz, 1H), 8.31 (dd, J=8.0, 1.5 Hz, 11H), 8.25 (dd, J=8.0, 1.5 Hz, 1H), 8.11 (d, J=8.0 Hz, 11H), 7.81 (s, 1H), 7.30 (d, J=8.0 Hz, 1H), 6.83 (s, 11H), 6.81 (s, 2H), 6.80 (s, 1H), 6.51 (s, 1H), 6.50 (s, 1H), 6.49 (s, 1H), 6.48 (s, 1H). See also Sun, et al. (1997). J Org Chem 62, 6469-6475, doi:DOI 10.1021/jo9706178.

### 7. Synthesis of Diacetyl-5-(2-carboxyethylaminocarbonyl) Oregon Green 488 (20)

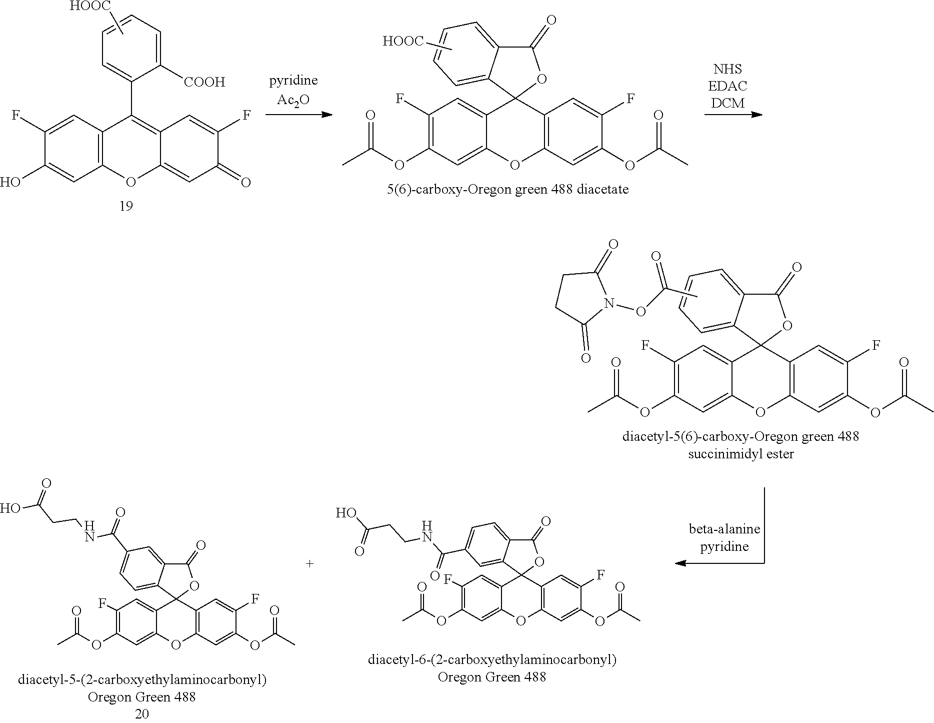

5(6)-carboxy-Oregon green (19) (167 mg) and pyridine (130 uL) were stirred in acetic anhydride (3.8 mL) at 80° C. for 5 min. The solvents were removed in vacuo to yield the crude 5(6)-carboxy-Oregon green diacetate (198 mg). A portion of the crude 5(6)-carboxy-Oregon green diacetate (30 mg) was stirred with N-hydroxysuccinimide (NHS) (8.3 mg, 1.2 equiv) and 1-Ethyl-3-(3-dimethylaminopropyl)carbodiimide (EDAC) (14 mg, 1.2 equiv) in dichloromethane (DCM) (2 mL) at room temperature for 3 h and then beta-alanine (54 mg, 10 equiv) and pyridine (2 mL) were added into the mixture which was further stirred at 40° C. overnight. The solvents were removed in vacuo and the residue was purified by preparative HPLC using a Luna 5 μm C18 column (isocratic, 35% MeCN in 0.1% TFA) to yield diacetyl-5-(2-carboxyethylaminocarbonyl) Oregon Green 488 (20) (15.2 mg) and diacetyl-6-(2-carboxyethylaminocarbonyl) Oregon Green 488 (13.4 mg).

diacetyl-5-(2-carboxyethylaminocarbonyl) Oregon Green 488 (20): yellow powder; ¹H NMR (500 MHz, methanol-d₄): δ 8.44 (d, J=1.6 Hz, 1H), 8.20 (dd, J=8.0, 1.6 Hz, 1H), 7.39 (d, J=8.0 Hz, 1H), 7.32 (s, 1H), 7.31 (s, 1H), 6.76 (s, 1H), 6.74 (s, 11H), 3.68 (t, J=6.8 Hz, 1H), 2.68 (t, J=6.8 Hz, 1H), 2.32 (s, 6H). See also Wu, X. L. et al. (2014) J Fluoresc 24, 775-786, doi:10.1007/s10895-014-1351-x.

### 8. Synthesis of Flu-Tacca-2 (4)

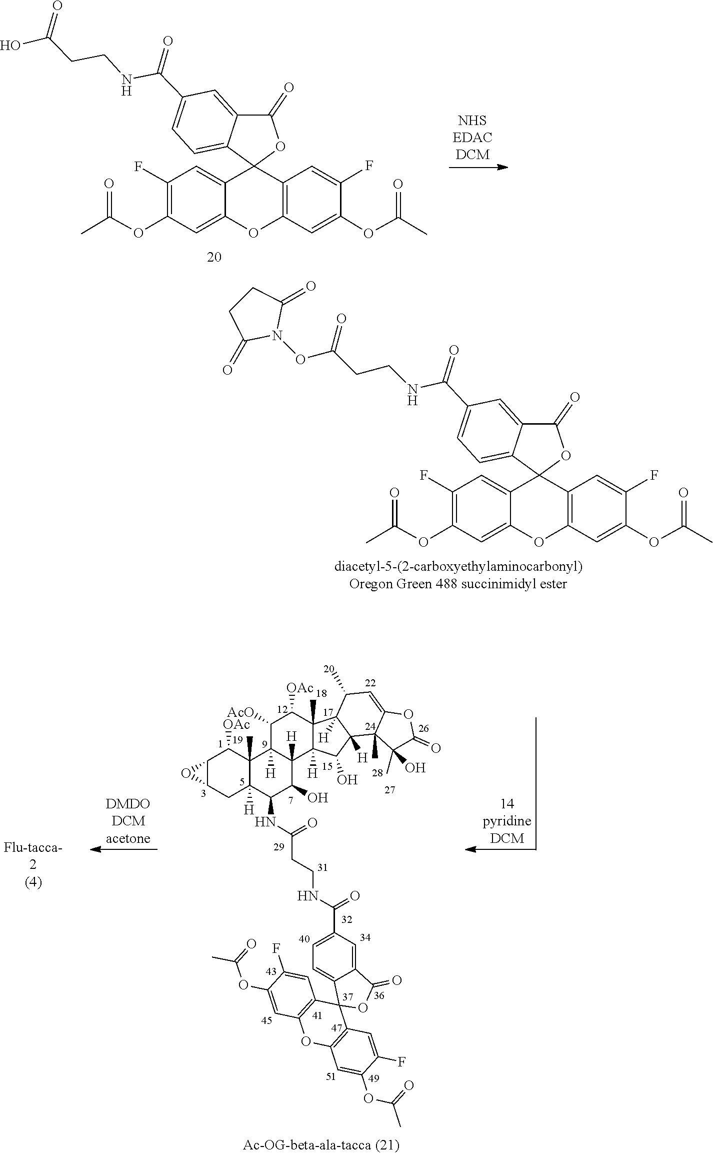

Compound 20 (24 mg) was stirred with NHS (5.6 mg, 1.2 equiv) and EDAC (7.8 mg, 1.2 equiv) in DCM (2 mL) at room temperature for 4 h followed by the removal of the solvent in vacuo. The residue was resuspended in saturated brine (2 mL) and partitioned with ethyl acetate (EtOAc) (4 mL×3). The combined organic layers were dried down in vacuo to yield the crude diacetyl-5-(2-carboxyethylaminocarbonyl) Oregon Green 488 succinimidyl ester which was directly mixed with 14 (11 mg) in a DCM (2 mL)/pyridine (2 mL) solution. The reactant mixture was further stirred at 40° C. for 24 h. The solvents were removed in vacuo and the residue was purified by semi-preparative HPLC using a Kinetex 5 μm F5 column (isocratic, 50% MeCN in 0.1% formic acid) to yield compound 21 (2.6 mg).

Ac-OG-beta-ala-tacca (21): light yellow solid; ¹H NMR data, see Table ii; ¹³C NMR (125 MHz, methanol-d₄) (some carbons were not detectable due to limited amount of material): δ 177.4, 175.3, 172.3, 171.6, 171.5, 169.4, 169.3, 168.1, 155.9, 153.1, 151.2, 148.4, 141.8, 138.5, 136.0, 127.5, 125.5, 125.4, 117.3, 117.2, 115.5, 115.4, 114.2, 83.9, 80.3, 75.6, 75.3, 73.6, 72.6, 72.5, 58.1, 54.1, 54.0, 51.9, 51.4, 49.2, 45.7, 42.1, 38.3, 38.1, 36.8, 36.4, 34.4, 32.2, 27.1, 25.4, 22.1, 21.5, 21.0, 20.7, 20.6, 20.2, 13.7, 13.5; HRESIMS (m/z): [M+Na]⁺ calcd. for C₆₂H₆₄F₂N₂NaO₂₁, 1233.3862; found 1233.3859.

Compound 21 (2.6 mg) was dissolved in 0.3 mL DCM and pre-chilled to −20° C. prior to the addition of 0.3 mL DMDO-acetone solution. The epoxidation reagent dimethyldioxirane (DMDO) was prepared as previous described⁵. The mixture was incubated at −20° C. for 4 h and then blown down by N₂. The residue was purified by semi-preparative HPLC using a Kinetex 5 μm F5 column (isocratic, 50% MeCN) to yield compound 4 (1.6 mg).

Flu-tacca-2 (4): light yellow solid; ¹H NMR data, see Table ii; HRESIMS (m/z): [M+Na]⁺ calcd. for C₆₂H₆₄F₂N₂NaO₂₂, 1249.3811; found 1249.3814.

### 9. Synthesis of Diacetyl-5-(2-carboxyethylaminocarbonyl) fluorescein (22)

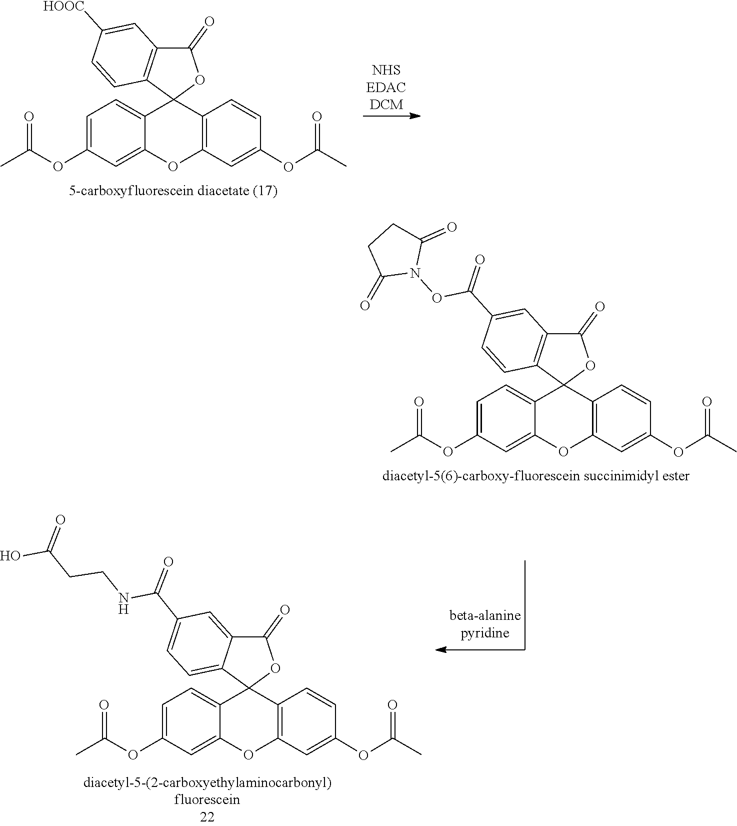 [SMILES: CC(=O)Oc1ccc2c(c1)Oc1cc(OC(C)=O)ccc1C21OC(=O)c2cc(C(=O)O)ccc21 ; CC(=O)Oc1ccc2c(c1)Oc1cc(OC(C)=O)ccc1C21OC(=O)c2cc(C(=O)ON3C(=O)CCC3=O)ccc21 ; CC(=O)Oc1ccc2c(c1)Oc1cc(OC(C)=O)ccc1C21OC(=O)c2cc(C(=O)NCCC(=O)O)ccc21]

5-Carboxyfluorescein diacetate (17) (28 mg) was stirred with NHS (8.3 mg, 1.2 equiv) and EDAC (14 mg, 1.2 equiv) in DCM (2 mL) at room temperature for 3 h and then beta-alanine (54 mg, 10 equiv) and pyridine (2 mL) were added into the mixture which was further stirred at 40° C. overnight. The solvents were removed in vacuo and the residue was purified by preparative HPLC using a Luna 5 μm C18 column (isocratic, 50% MeCN in 0.1% formic acid) to yield diacetyl-5-(2-carboxyethylaminocarbonyl) fluorescein (22) (20 mg).

Diacetyl-5-(2-carboxyethylaminocarbonyl) fluorescein (22): light yellow powder; ¹H NMR (400 MHz, methanol-d₄): δ 8.43 (d, J=1.6 Hz, 1H), 8.17 (dd, J=8.0, 1.6 Hz, 1H), 7.30 (d, J=8.0 Hz, 1H), 7.16 (s, 2H), 6.86 (m, 4H), 3.67 (t, J=6.8 Hz, 1H), 2.67 (t, J=6.8 Hz, 1H), 2.27 (s, 6H); ¹³C NMR (100 MHz, methanol-d₄): δ 170.5, 170.0, 168.2, 156.6, 154.0, 152.8, 138.2, 135.9, 130.0, 127.7, 125.5, 125.1, 119.4, 117.1, 111.6, 83.3, 37.3, 20.9. See also Wu, X. L. et al. (2014) J Fluoresc 24, 775-786, doi:10.1007/s10895-014-1351-x.

### 10. Synthesis of Flu-Tacca-3 (5)

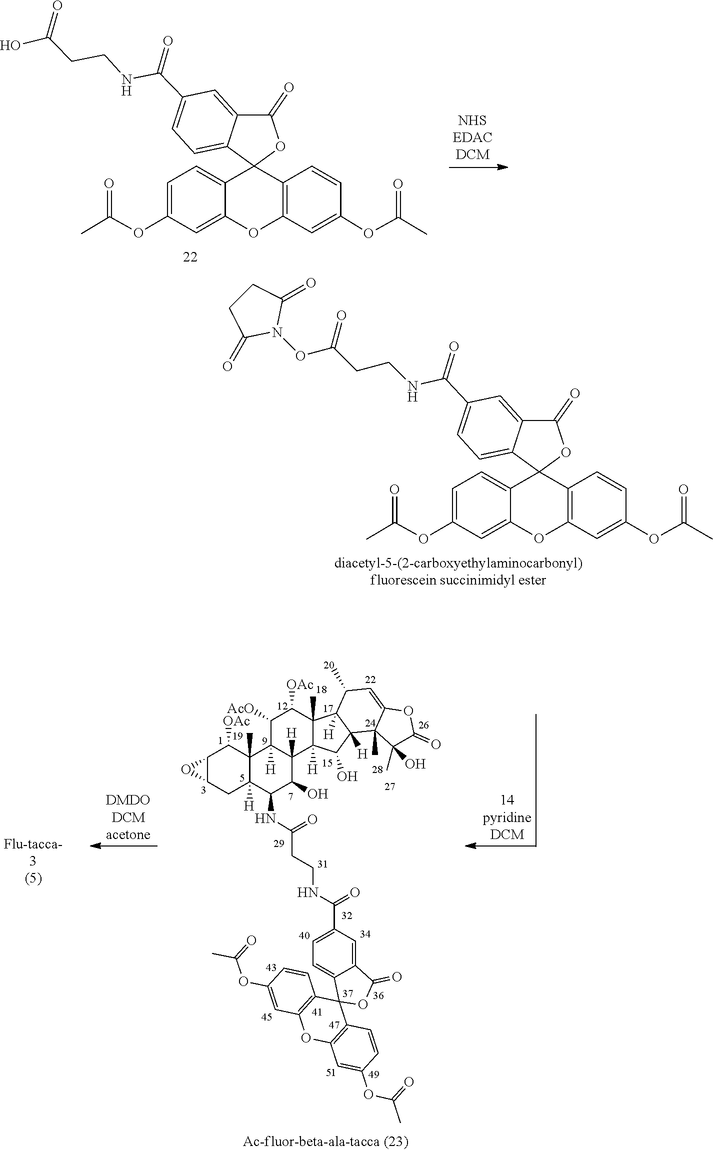

Compound 22 (20 mg) was stirred with NHS (5.2 mg, 1.2 equiv) and EDAC (8.7 mg, 1.2 equiv) in DCM (2 mL) at room temperature for 4 h followed by the removal of the solvent in vacuo. The residue was resuspended in saturated brine (2 mL) and partitioned with EtOAc (4 mL×3). The combined organic layers were dried down in vacuo to yield the crude diacetyl-5-(2-carboxyethylaminocarbonyl) fluorescein succinimidyl ester (24 mg) which was directly mixed with 14 (9 mg) in a DCM (2 mL)/pyridine (2 mL) solution. The reactant mixture was further stirred at 40° C. overnight. The solvents were removed in vacuo and the residue was purified by semi-preparative HPLC using a Kinetex 5 μm F5 column (isocratic, 45% MeCN in 0.1% formic acid) to yield compound 23 (5.0 mg).

Ac-fluor-beta-ala-tacca (23): light yellow solid; ¹H NMR data, see Table iii; ¹³C NMR (125 MHz, methanol-d₄): δ 177.3, 175.3, 172.3, 171.6, 171.5, 170.5, 170.4, 169.9, 168.0, 156.0, 155.9, 154.1, 152.8, 138.0, 135.9, 129.9, 129.8, 127.7, 125.6, 125.1, 119.5, 119.4, 117.0, 112.1, 111.8, 111.7, 83.3, 80.3, 75.6, 75.4, 73.6, 72.6, 72.5, 58.1, 54.1, 54.0, 51.9, 51.4, 49.3, 45.7, 42.1, 38.3, 38.1, 36.8, 36.4, 34.4, 32.2, 27.1, 25.4, 22.1, 21.5, 21.0, 20.9, 20.7, 20.6, 13.7, 13.5; HRESIMS (m/z): [M+Na]¹ calcd. for C₆₂H₆₆N₂NaO₂₁, 1197.4050; found 1197.4055.

Compound 23 (5.0 mg) was epoxidized in 1 mL DMDO-acetone/DCM (1:1) solution as previously described. The product was purified by semi-preparative HPLC using a Kinetex 5 μm F5 column (isocratic, 45% MeCN) to yield compound 5 (4.2 mg).

Flu-tacca-3 (5): light yellow solid; ¹H NMR data, see Table viii; HRESIMS (m/z): [M+Na]⁺ calcd. for C₆₂H₆₆N₂NaO₂₂, 1213.3999; found 1213.4006.

### 11. Synthesis of Flu-Tacca-4 (6)

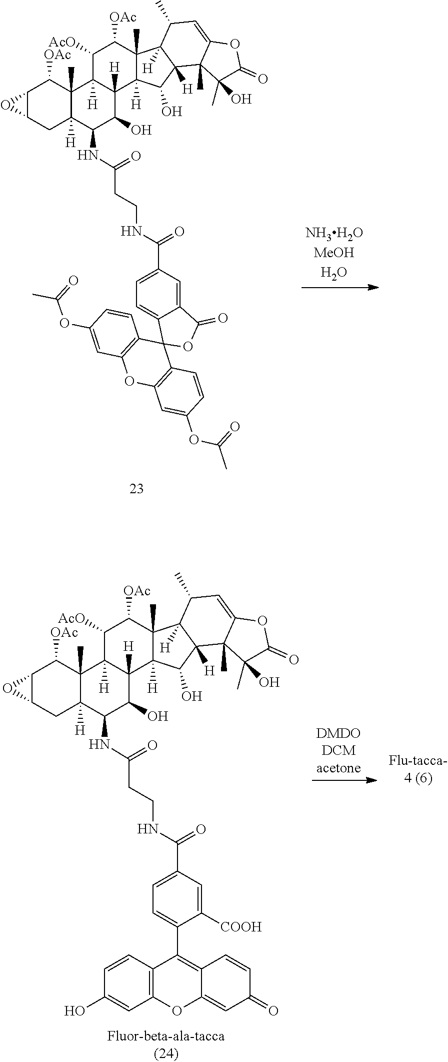

Compound 23 (11 mg) was dissolved in 3 mL MeOH/H₂O (2:1) followed by the addition of 50 μL ammonia hydroxide (NH₃.H₂O). The mixture was stirred at room temperature for 1 h and then the solvents were removed in vacuo. The residue was purified by preparative HPLC using a Kinetex 5 μm F5 column (isocratic, 40% MeCN in 0.1% TFA) to yield compound 24 (9.5 mg).

Fluor-beta-ala-tacca (24): yellow solid; ¹H NMR data, see Table iv; HRESIMS (m/z): [M+Na]⁺ calcd. for C₅₈H₆₂N₂NaO₁₉, 1113.3839; found 1113.3830.

Compound 24 (6.0 mg) was epoxidized in 1 mL DMDO-acetone/DCM (1:1) solution as previously described. The product was purified by preparative HPLC using a Kinetex 5 μm F5 column (isocratic, 45% MeCN in 0.1% TFA) to yield compound 6 (5.2 mg).

Flu-tacca-4 (6): yellow solid; ¹H NMR data, see Table iv; HRESIMS (m/z): [M+Na]⁴ calcd. for C₅₈H₆₂N₂NaO₂₀, 1129.3788; found 1129.3799.

### 12. Synthesis of Dipivalyl-5-(2-carboxyethylaminocarbonyl) fluorescein (25)

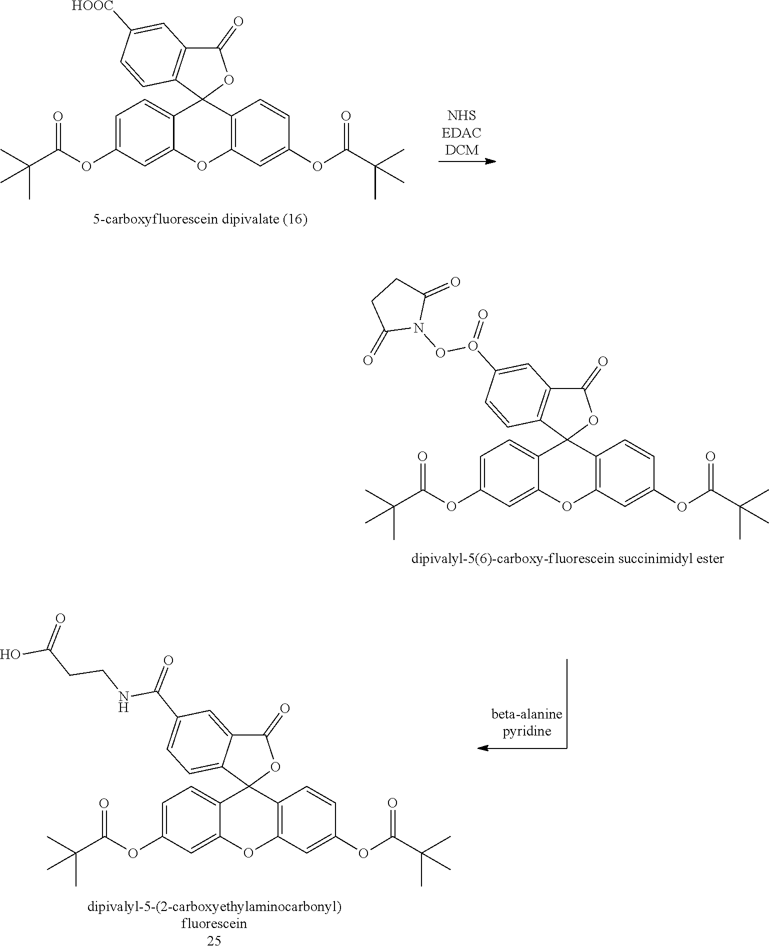

5-Carboxyfluorescein dipivalate (16) (33 mg) was stirred with NHS (8.3 mg, 1.2 equiv) and EDAC (14 mg, 1.2 equiv) in DCM (2 mL) at room temperature for 3 h and then beta-alanine (54 mg, 10 equiv) and pyridine (2 mL) were added into the mixture which was further stirred at 40° C. overnight. The solvents were removed in vacuo and the residue was purified by preparative HPLC using a Luna 5 μm C18 column (isocratic, 85% MeCN in 0.1% formic acid) to yield dipivalyl-5-(2-carboxyethylaminocarbonyl) fluorescein (25) (27 mg).

Dipivalyl-5-(2-carboxyethylaminocarbonyl) fluorescein (25): white powder; ¹H NMR (400 MHz, methanol-d₄): δ 8.44 (d, J=1.6 Hz, 1H), 8.17 (dd, J=8.0, 1.6 Hz, 1H), 7.30 (d, J=8.0 Hz, 1H), 7.13 (d, J=2.1 Hz, 2H), 6.86 (d, J=8.7 Hz, 2H), 6.86 (d, J=8.7 Hz, 2H), 6.83 (dd, J=8.7, 2.1 Hz, 2H), 3.67 (t, J=6.8 Hz, 1H), 2.67 (t, J=6.8 Hz, 1H), 1.34 (s, 18H); ¹³C NMR (100 MHz, methanol-d₄): δ 177.9, 169.9, 168.1, 156.6, 154.3, 152.8, 138.1, 135.9, 130.1, 127.8, 125.5, 125.1, 119.3, 117.1, 111.5, 83.3, 40.2, 37.3, 27.4. See also Wu, X. L. et al. (2014) J Fluoresc 24, 775-786, doi:10.1007/s10895-014-1351-x.

### 13. Synthesis of Flu-Tacca-5 (8)

Compound 25 (27 mg) was stirred with NHS (6.0 mg, 1.2 equiv) and EDAC (10 mg, 1.2 equiv) in DCM (2 mL) at room temperature for 4 h followed by the removal of the solvent in vacuo. The residue was resuspended in saturated brine (2 mL) and partitioned with EtOAc (4 mL×3). The combined organic layers were dried down in vacuo to yield the crude dipivalyl-5-(2-carboxyethylaminocarbonyl) fluorescein succinimidyl ester which was directly mixed with 14 (11 mg) in a DCM (2 mL)/pyridine (2 mL) solution. The reactant mixture was further stirred at 40° C. overnight. The solvents were removed in vacuo and the residue was purified by semi-preparative HPLC using a Kinetex 5 μm F5 column (isocratic, 55% MeCN in 0.1% formic acid) to yield compound 7 (6.8 mg).

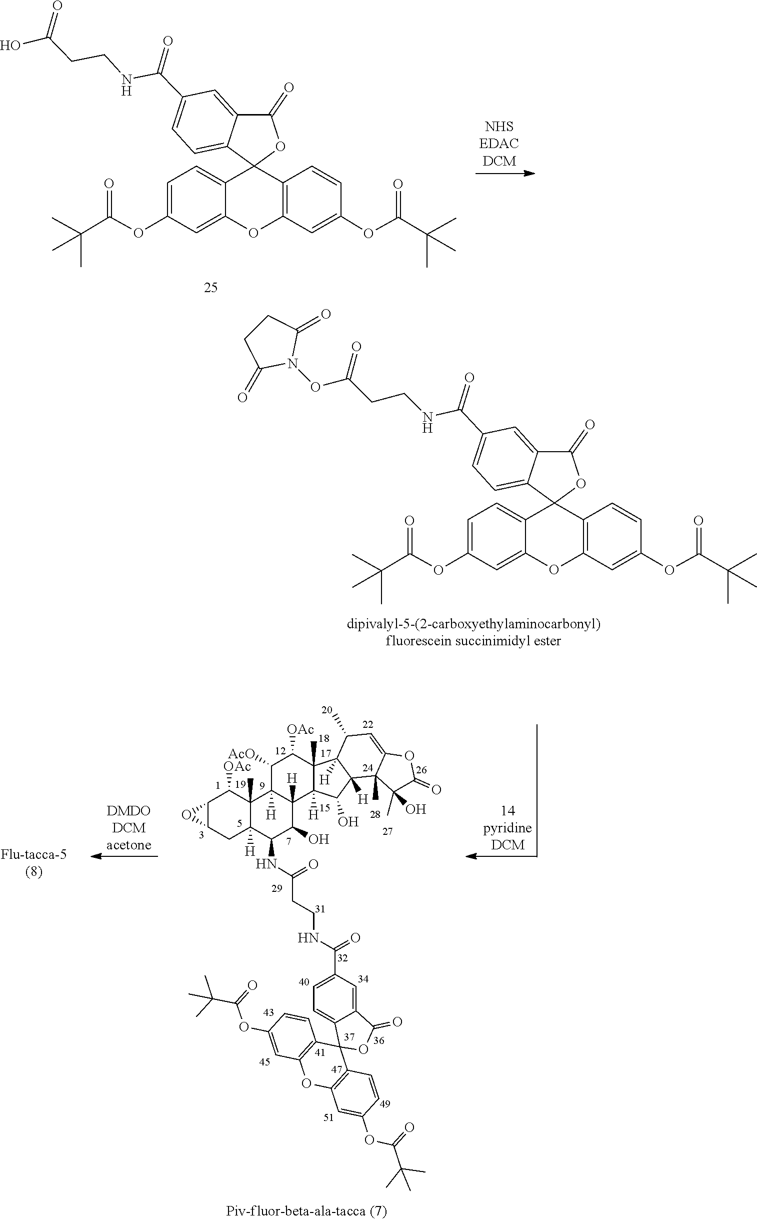

Piv-fluor-beta-ala-tacca (7): white solid; ¹H NMR data, see Table v; ¹³C NMR (125 MHz, methanol-d₄): δ 177.9 (2C), 177.3, 175.3, 172.4, 171.5, 171.4, 169.9, 167.9, 156.9, 155.9, 154.4, 152.8, 138.0, 135.9, 130.0, 129.9, 127.8, 125.7, 125.0, 119.4, 119.3, 117.0, 116.9, 112.1, 111.7, 111.6, 83.4, 80.3, 75.6, 75.4, 73.6, 72.6, 72.5, 58.1, 54.1, 54.0, 51.9, 51.3, 49.3, 45.7, 42.1, 40.2, 38.3, 38.1, 36.8, 36.4, 34.4, 32.2, 27.4, 27.1, 25.4, 22.1, 21.6, 21.0, 20.7, 20.6, 13.8, 13.6; HRESIMS (m/z): [M+Na]⁺ calcd. for C₆₈H₇₈N₂NaO₂₁, 1281.4989; found 1281.5015.

Compound 7 (6.0 mg) was epoxidized in 1 mL DMDO-acetone/DCM (1:1) solution as previously described. The product was purified by semi-preparative HPLC using a Kinetex 5 μm F5 column (isocratic, 65% MeCN) to yield compound 8 (5.8 mg).

Flu-tacca-5 (8): light yellow solid; ¹H NMR data, see Table v; HRESIMS (m/z): [M+Na]⁺ calcd. for C₆₈H₇₈N₂NaO₂₂, 1297.4938; found 1297.4880.

### 14. Synthesis of Fmoc-Gly-Tacca (26)

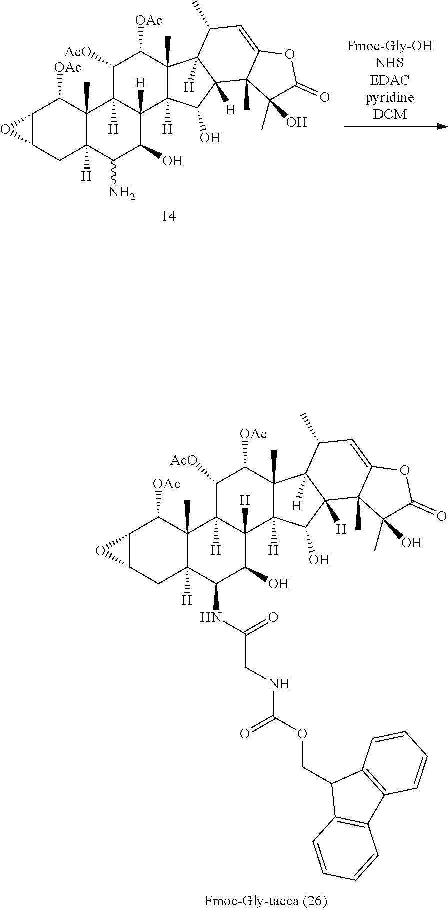

Compound 14 (10 mg) was stirred with Fmoc-Gly-OH (6.7 mg, 1.5 equiv), NHS (2.7 mg, 1.5 equiv) and EDAC (4.3 mg, 1.5 equiv) in 2 mL DCM/pyridine (1:1) at room temperature overnight followed by the removal of the solvents in vacuo. The residue was re-dissolved in MeCN and purified by semi-preparative HPLC using a Kinetex 5 μm F5 column (isocratic, 50% acetonitrile in 0.1% formic acid) to yield compound 26 (4.5 mg).

Fmoc-Gly-tacca (26): white solid; ¹H NMR (500 MHz, methanol-d₄): δ 7.79 (d, J=7.4 Hz, 2H), 7.65 (d, J=7.4 Hz, 2H), 7.39 (t, J=7.4 Hz, 2H), 7.32 (d, J=7.4 Hz, 2H), 5.27 (dd, J=11.8, 2.8 Hz, 1H), 5.23 (d, J=2.8 Hz, 1H), 4.63 (d, J=5.6 Hz, 1H), 4.40 (m, 2H), 4.32 (m, 1H), 4.27 (m, 1H), 4.24 (d, J=6.7 Hz, 1H), 3.83 (m, 2H), 3.63 (dd, J=10.1, 4.4 Hz, 1H), 3.35 (m, 1H), 3.25 (m, 1H), 2.42 (dd, J=13.4, 10.4 Hz, 1H), 2.25 (t, J=10.9 Hz, 1H), 2.19 (m, 1H), 2.09 (s, 3H), 2.07 (s, 3H), 1.99 (m, 1H), 1.96 (m, 1H), 1.95 (m, 1H), 1.91 (s, 3H), 1.81 (dd, J=13.4, 9.6 Hz, 1H), 1.61 (s, 3H), 1.24 (s, 3H), 0.98 (s, 3H), 0.90 (s, 3H), 0.88 (d, J=6.4 Hz, 3H); ¹³C NMR (125 MHz, methanol-d₄): δ 177.3, 173.6, 171.6, 171.5, 159.4, 155.9, 145.2, 145.1, 142.6, 128.9, 128.3, 128.2, 126.2, 126.1, 121.0, 112.0, 80.3, 75.7, 75.2, 73.7, 72.5, 72.4, 68.3, 57.9, 54.3, 54.1, 51.9, 51.4, 49.3, 48.3, 45.7, 45.2, 42.0, 38.3, 36.5, 34.2, 32.2, 26.9, 25.4, 22.1, 21.5, 21.0, 20.7, 20.6, 14.0, 13.5; HRESIMS (m/z): [M+Na]⁺ calcd. for C₅₁H₆₀N₂NaO₁₅, 963.3886; found 963.3892. See also Lee, et al. (2017) Angew Chem Int Edit 56, 6927-6931, doi:10.1002/anie.201703298.

### 15. Synthesis of Flu-Tacca-6 (9)

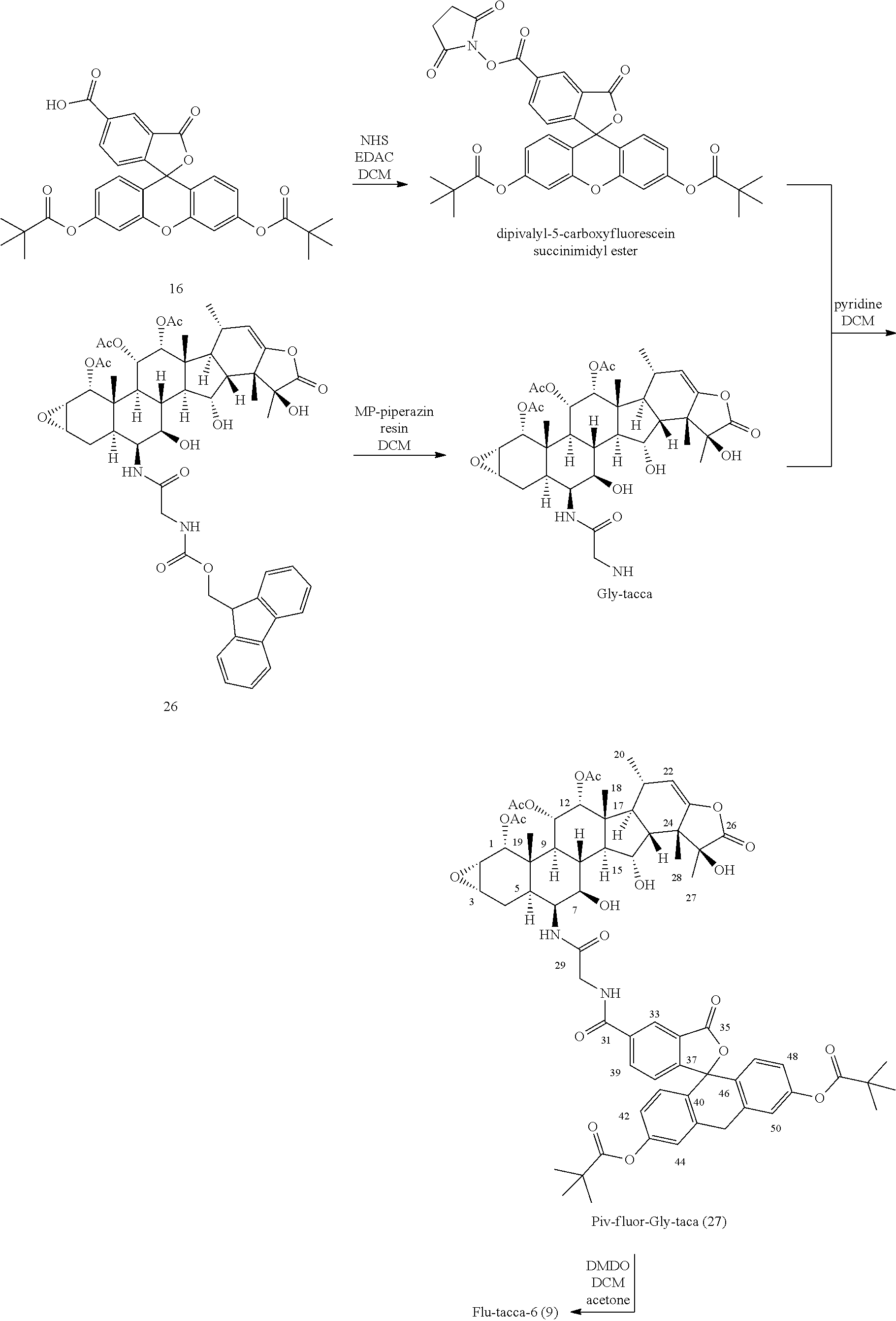

Compound 26 (10 mg) was stirred with MP-piperazine resin (200 mg) in DCM (1 mL) at 40° C. for 24 h followed by the removal of the solvent in vacuo. The residue was washed with MeOH (2 mL×3) and the combined MeOH wash was dried down in vacuo to yield the crude Gly-tacca which was directly mixed with the crude dipivalyl-5-carboxyfluorescein succinimidyl ester (13 mg) (prepared from 16 using the same method as previously described) in 2 mL DCM/pyridine (1:1). The reactant mixture was further stirred at 40° C. overnight. The solvents were removed in vacuo and the residue was purified by semi-preparative HPLC using a Kinetex 5 μm Biphenyl column (isocratic, 65% MeCN in 0.1% formic acid) to yield compound 27 (3.0 mg).

Piv-fluor-Gly-tacca (27): white solid; ¹H NMR data, see Table vi; ¹³C NMR (125 MHz, methanol-d₄) (some carbons were not detectable due to limited amount of material): δ 177.9, 177.3, 172.9, 172.3, 171.6, 171.5, 169.9, 168.8, 157.0, 155.9, 154.4, 152.8, 137.5, 136.0, 130.0, 127.8, 125.7, 125.3, 119.4, 117.0, 112.1, 111.6, 83.4, 80.3, 75.7, 75.3, 73.8, 72.5, 72.4, 57.9, 54.5, 54.1, 51.9, 51.5, 51.3, 49.1, 45.7, 44.8, 42.1, 40.2, 38.4, 36.6, 34.3, 32.2, 27.4, 27.0, 25.4, 22.1, 21.5, 21.0, 20.7, 20.6, 14.1, 13.6; HRESIMS (m/z): [M+Na]⁺ calcd. for C₆₇H₇₆N₂NaO₂₁, 1267.4833; found 1267.4845.

Compound 27 (3.0 mg) was epoxidized in 0.6 mL DMDO-acetone/DCM (1:1) solution as previously described. The product was purified by semi-preparative HPLC using a Kinetex 5 μm F5 column (isocratic, 65% MeCN) to yield compound 9 (1.8 mg).

Flu-tacca-6 (9): light yellow solid; ¹H NMR data, see Table vi; HRESIMS (m/z): [M+Na]⁺ calcd. for C₆₇H₇₆N₂NaO₂₂, 1283.4782; found 1283.4718.

### 16. Synthesis of Flu-Tacca-7 (11)

Compound 14 (10 mg) was stirred with 16 (13 mg, 1.5 equiv) and HATU (5.5 mg, 1.5 equiv) in 2 mL EtOAc/pyridine (1:1) at 35° C. overnight. The solvents were removed in vacuo and the residue was purified by preparative HPLC using a Kinetex 5 μm F5 column (isocratic, 70% MeCN in 0.1% formic acid) to yield compound 10 (3.8 mg).

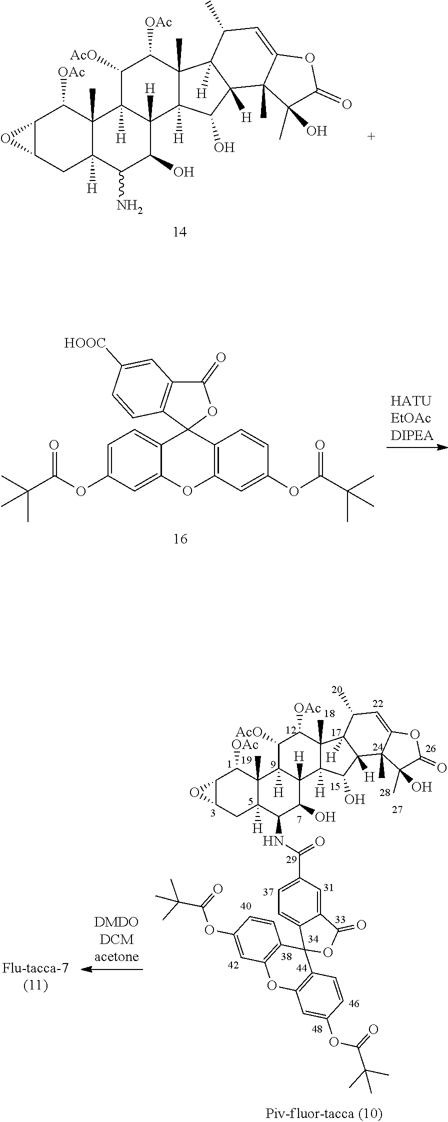

Piv-fluor-tacca (10): white solid; ¹H NMR data, see Table vii; ¹³C NMR (125 MHz, acetonitrile-d₃) (some carbons were not detectable due to limited amount of material): δ 177.5, 176.6, 171.4, 171.0, 170.7, 169.1, 156.3, 155.6, 154.0, 152.4, 138.2, 136.2, 130.0, 127.1, 125.4, 125.0, 119.3, 116.9, 111.6, 111.5, 82.5, 80.0, 75.0, 74.6, 74.1, 72.2, 72.0, 57.5, 54.7, 53.4, 51.7, 51.0, 51.7, 48.6, 45.3, 41.6, 39.8, 38.0, 36.5, 33.8, 31.9, 27.3, 26.6, 25.4, 22.4, 21.6, 21.1, 20.8, 20.4, 14.4, 13.7; HRESIMS (m/z): [M+Na]⁺ calcd. for C₆₅H₇₃NNaO₂₀, 1210.4618; found 1210.4676.

Compound 10 (3.0 mg) was epoxidized in 0.6 mL DMDO-acetone/DCM (1:1) solution as previously described. The product was purified by semi-preparative HPLC using a Kinetex 5 μm F5 column (isocratic, 70% MeCN) to yield compound 11 (1.4 mg).

Flu-tacca-7 (11): light yellow solid; ¹H NMR data, see Table vii; HRESIMS (m/z): [M+Na]^{t} calcd. for C₆₅H₇₃NNaO₂₁, 1226.4567; found 1226.4578.

### 17. Synthesis of Flu-Tacca-8 (12)

Compound 14 (9.5 mg) was stirred with 17 (9.9 mg, 1.5 equiv) and HATU (8.2 mg, 1.5 equiv) in 2 mL EtOAc/pyridine (1:1) at 35° C. overnight. The solvents were removed in vacuo to yield the crude Ac-fluor-tacca which was re-dissolved in 3 mL MeOH/H₂O (2:1) followed by the addition of 50 μL ammonia hydroxide (NH₃.H₂O). The mixture was stirred at room temperature for 1 h and then the solvents were removed in vacuo. The residue was purified by preparative HPLC using a Kinetex 5 μm F5 column (isocratic, 45% MeCN in 0.1% TFA) to yield compound 28 (7.1 mg).

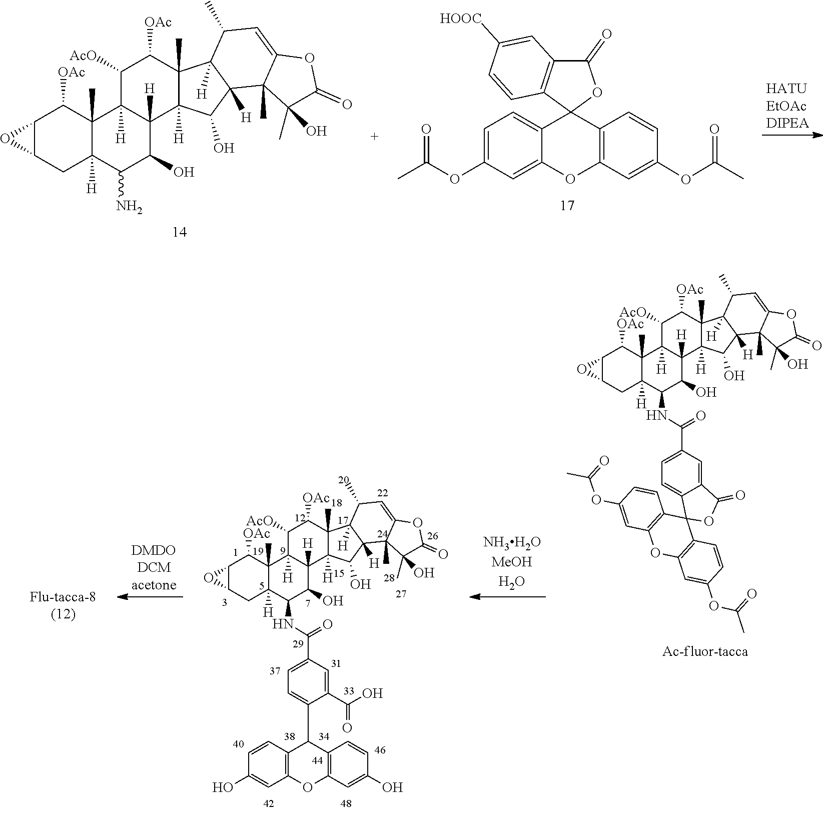

Fluor-tacca (28): yellow solid; ¹H NMR data, see Table viii; HRESIMS (m/z): [M+Na]⁺ calcd. for C₅₅H₅₇NNaO₁₉, 1042.3468; found 1042.3427.

Compound 28 (7.0 mg) was epoxidized in 1 mL DMDO-acetone/DCM (1:1) solution as previously described. The product was purified by preparative HPLC using a Kinetex 5 μm F5 column (isocratic, 50% MeCN in 0.1% TFA) to yield compound 12 (6.2 mg).

Flu-tacca-8 (12): yellow solid; ¹H NMR data, see Table viii; HRESIMS (m/z): [M+Na]⁺ calcd. for C₅₅H₅₇NNaO₁₈, 1058.3417; found 1058.34176.

### 18. Compound Characterization and Purity

Both 1D (¹H and ¹³C) and 2D (¹H-¹H COSY, HSQC, and HMBC) NMR data were collected for key new compounds to confirm their structures and assign the ¹H NMR data (Tables i-viii). In case of new compounds with sensitive structural features (i.e., 22,23-epoxide) and/or low quantities (<2 mg), time-consuming ¹³C NMR data were not collected to avoid potential structural decompositions under the NMR conditions. Instead, besides ¹H NMR data, the fast 2D NMR data (¹H-¹H COSY and HSQC) were also collected to confirm their structures and unambiguously assign their ¹H NMR data (Table ii-viii). TD and 2D NMR spectra for all synthetic compounds were attached as references and evidence of compound purity (95% or better). HRFSIMS data were collected and listed for key new compounds to confirm their identity

TABLE I

| No. | 6S-¹³C | 6S-¹H (J) | 6R-¹³C | 6R-¹H (J) |
|---|---|---|---|---|
| 1 | 75.2 | 4.69, d (5.4) | 73.9 | 4.74, d (5.6) |
| 2 | 51.2 | 3.43, m | 51.1 | 3.47, m |
| 3 | 53.4 | 3.40, m | 52.9 | 3.38, m |
| 4 | 26.4 | 2.19, m | 26.6 | 2.25, m |
| | | 2.02, m | | 1.83, m |
| 5 | 32.1 | 2.17, m | 34.2 | 1.90, m |
| 6 | 57.8 | 3.46, m | 58.0 | 2.97, t (11.6) |
| 7 | 71.6 | 3.78, m | 74.3 | 3.32, m |
| 8 | 35.9 | 2.05, m | 40.1 | 1.88, m |
| 9 | 41.5 | 2.34, m | 41.6 | 2.31, m |
| 10 | 37.9 | | 39.4 | |
| 11 | 72.3 | 5.30, dd | 72.1 | 5.32, dd |
| | | (11.6, 2.9) | | (11.7, 2.8) |
| 12 | 75.0 | 5.25, m | 75.2 | 5.24, m |
| 13 | 45.6 | | 45.2 | |
| 14 | 57.3 | 2.05, m | 57.8 | 2.00, m |
| 15 | 72.1 | 4.49, m | 72.1 | 4.49, m |
| 16 | 51.9 | 2.50, dd | 51.6 | 2.51, dd |
| | | (9.9, 13.6) | | (10.0, 13.5) |
| 17 | 49.2 | 1.82, m | 49.2 | 1.82, m |
| 18 | 13.5 | 1.03, s | 13.3 | 1.00, s |
| 19 | 13.8 | 0.97, s | 13.8 | 0.97, s |
| 20 | 20.6 | 0.90, d (7.1) | 20.5 | 0.89, d (7.1) |
| 21 | 31.9 | 2.23, m | 31.9 | 2.23, m |
| 22 | 112.1 | 5.04, m | 112.1 | 5.04, m |
| 23 | 156.4 | | 156.6 | |
| 24 | 51.3 | | 51.5 | |
| 25 | 80.1 | | 80.1 | |
| 26 | 177.5 | | 177.4 | |
| 27 | 21.4 | 1.64, s | 21.4 | 1.63, s |
| 28 | 25.4 | 1.32, s | 25.3 | 1.32, s |
| 1-OAc | 171.3 | | 171.3 | |
| | 20.6 | 2.10, s | 20.6 | 2.10, s |
| 11-OAc | 172.2 | | 172.2 | |
| | 21.4 | 1.91, s | 21.6 | 1.91, s |
| 12-OAc | 171.4 | | 171.4 | |
| | 20.9 | 2.08, s | 20.9 | 2.08, s |

TABLE II

| No. | 21 | 4 |
|---|---|---|
| 1 | 4.61, d (5.6) | 4.60, d (5.6) |
| 2 | 3.29, m | 3.29, m |
| 3 | 3.21, m | 3.20, m |
| 4 | 1.99, m | 2.00, m |
| | 1.89, m | 1.86, m |
| 5 | 1.96, m | 1.94, m |
| 6 | 4.33, br t (4.1) | 4.32, br t (4.0) |
| 7 | 3.65, dd (10.3, 4.1) | 3.66, dd (10.5, 4.0) |
| 8 | 2.21, m | 2.17, m |
| 9 | 2.25, t (11.1) | 2.24, t (11.1) |
| 11 | 5.28, dd (11.1, 2.8) | 5.24, br d (11.1) |
| 12 | 5.22, d (2.8) | 5.14, br s |
| 14 | 1.96, m | 1.94, m |
| 15 | 4.39, dd (10.4, 8.4) | 4.29, br t (8.4) |
| 16 | 2.47, dd (13.4, 10.4) | 2.07, m |
| 17 | 1.82, dd (13.4, 9.8) | 2.07, m |
| 18 | 1.04, s | 0.86, s |
| 19 | 0.89, s | 0.92, s |
| 20 | 0.89, d (7.1) | 0.99, d (7.2) |
| 21 | 2.20, m | 1.55, m |
| 22 | 5.00, d (1.6) | 3.33, d (1.3) |
| 27 | 1.62, s | 1.73, s |
| 28 | 1.24, s | 1.22, s |
| 30 | 2.79, m | 2.78, m |
| | 2.67, m | 2.66, m |
| 31 | 3.74, m | 3.73, m |
| 34 | 8.47, d (1.6) | 8.47, s |
| 39 | 8.23, dd (8.1, 1.6) | 8.23, d (8.1) |
| 40 | 7.39, d (8.1) | 7.39, d (8.1) |
| 42, 48 | 6.72, d (4.9) | 6.72, d (4.9) |
| | 6.70, d (4.9) | 6.70, d (4.9) |
| 45, 51 | 7.32, s | 7.33 s |
| | 7.31, s | 7.32, s |
| 1-OAc | 2.09, s | 2.09, s |
| 11-OAc | 1.89, s | 1.88, s |
| 12-OAc | 2.07, s | 2.09, s |
| 44,50-OAc | 2.32, s | 2.32, s |

TABLE III

| No. | 23 | 5 |
|---|---|---|
| 1 | 4.57, d (5.6) | 4.56, d (5.6) |
| 2 | 3.26, dd (5.6, 3.8) | 3.25, dd (5.6, 3.8) |
| 3 | 3.14, m | 3.13, m |
| 4 | 1.99, m | 2.00, m |
| | 1.87, m | 1.86, m |
| 5 | 1.95, m | 1.95, m |
| 6 | 4.33, br t (4.6) | 4.32, br t (4.0) |
| 7 | 3.65, dd (10.2, 4.6) | 3.66, dd (10.5, 4.0) |
| 8 | 2.21, m | 2.17, m |
| 9 | 2.25, t (11.2) | 2.23, t (11.5) |
| 11 | 5.29, dd (11.2, 2.9) | 5.24, dd (11.5, 2.8) |
| 12 | 5.22, d (2.9) | 5.13, d (2.8) |
| 14 | 1.96, m | 1.95, m |
| 15 | 4.39, dd (10.4, 8.4) | 4.29, br t (8.4) |
| 16 | 2.48, dd (13.5, 10.4) | 2.07, m |
| 17 | 1.82, dd (13.4, 9.8) | 2.09, m |
| 18 | 1.05, s | 0.82, s |
| 19 | 0.84, s | 0.92, s |
| 20 | 0.89, d (7.1) | 0.99, d (7.4) |
| 21 | 2.20, m | 1.55, m |
| 22 | 5.01, d (1.6) | 3.33, d (1.3) |
| 27 | 1.62, s | 1.73, s |
| 28 | 1.24, s | 1.22, s |
| 30 | 2.82, m | 2.81, m |
| | 2.65, m | 2.64, m |
| 31 | 3.74, m | 3.74, m |
| 34 | 8.48, d (1.6) | 8.47, d (1.6) |
| 39 | 8.23, dd (8.1, 1.6) | 8.23, dd (8.1, 1.6) |
| 40 | 7.36, d (8.1) | 7.36, d (8.1) |
| 42, 48 | 6.85, d (8.7) | 6.85, d (8.7) |
| | 6.83, d (8.7) | 6.83, d (8.7) |
| 43, 49 | 6.89, dd (8.7, 2.3) | 6.89, dd (8.7, 2.3) |
| | 6.91, dd (8.7, 2.3) | 6.91, dd (8.7, 2.3) |
| 45, 51 | 7.20, d (2.3) | 7.21, d (2.3) |
| | | 7.22, d (2.4) |
| 1-OAc | 2.09, s | 2.09, s |
| 11-OAc | 1.89, s | 1.88, s |
| 12-OAc | 2.07, s | 2.09, s |
| 44,50-OAc | 2.28, s | 2.28, s |

TABLE IV

| No. | 24 | 6 |
|---|---|---|
| 1 | 4.59, d (5.6) | 4.58, d (5.6) |
| 2 | 3.28, m | 3.28, m |
| 3 | 3.17, m | 3.17, m |
| 4 | 1.99, m | 1.99, m |
| | 1.88, m | 1.87, m |
| 5 | 1.96, m | 1.96, m |
| 6 | 4.34, br t (4.6) | 4.33, br t (4.6) |
| 7 | 3.66, dd (10.2, 4.6) | 3.66, dd (10.5, 4.6) |
| 8 | 2.21, m | 2.17, m |
| 9 | 2.26, t (11.2) | 2.25, t (11.2) |
| 11 | 5.31, dd (11.2, 2.9) | 5.26, dd (11.2, 2.8) |
| 12 | 5.22, d (2.9) | 5.14, d (2.8) |
| 14 | 1.97, m | 1.95, m |
| 15 | 4.39, dd (10.5, 8.4) | 4.29, br t (8.6) |
| 16 | 2.46, dd (13.5, 10.5) | 2.07, m |
| 17 | 1.82, dd (13.5, 9.9) | 2.09, m |
| 18 | 1.05, s | 0.86, s |
| 19 | 0.88, s | 0.94, s |
| 20 | 0.89, d (7.1) | 1.00, d (7.4) |
| 21 | 2.20, m | 1.56, m |
| 22 | 5.01, d (1.6) | 3.34, s |
| 27 | 1.62, s | 1.73, s |
| 28 | 1.23, s | 1.22, s |
| 30 | 2.82, m | 2.82, m |
| | 2.67, m | 2.67, m |
| 31 | 3.76, m | 3.75, m |
| 34 | 8.56, s | 8.54, s |
| 39 | 8.25, dd (8.1, 1.6) | 8.24, dd (8.1, 1.6) |
| 40 | 7.39, d (8.1) | 7.38, d (8.1) |
| 42, 48 | 6.83, d (9.0) | 6.79, m |
| | 6.80, d (9.0) | |
| 43, 49 | 6.73, d (9.0) | 6.72, m |
| 45, 51 | 6.88, s | 6.87, br s |
| 1-OAc | 2.09, s | 2.10, s |
| 11-OAc | 1.89, s | 1.89, s |
| 12-OAc | 2.07, s | 2.10, s |

TABLE V

| No. | 7 | 8 |
|---|---|---|
| 1 | 4.56, d (5.6) | 4.54, d (5.6) |
| 2 | 3.25, dd (5.6, 3.8) | 3.24, dd (5.6, 3.8) |
| 3 | 3.13, m | 3.12, m |
| 4 | 1.98, m | 1.98, m |
| | 1.87, m | 1.87, m |
| 5 | 1.96, m | 1.96, m |
| 6 | 4.32, br t (4.6) | 4.32, br t (4.6) |
| 7 | 3.65, dd (10.2, 4.6) | 3.65, dd (10.5, 4.6) |
| 8 | 2.20, m | 2.15, m |
| 9 | 2.24, t (11.2) | 2.23, t (11.2) |
| 11 | 5.28, dd (11.2, 2.9) | 5.24, dd (11.2, 2.8) |
| 12 | 5.21, d (2.9) | 5.13, d (2.8) |
| 14 | 1.95, m | 1.95, m |
| 15 | 4.38, dd (10.5, 8.4) | 4.28, br t (8.8) |
| 16 | 2.46, dd (13.5, 10.5) | 2.07, m |
| 17 | 1.81, dd (13.5, 9.7) | 2.09, m |
| 18 | 1.04, s | 0.81, s |
| 19 | 0.82, s | 0.92, s |
| 20 | 0.87, d (7.1) | 0.99, d (7.4) |
| 21 | 2.20, m | 1.54, m |
| 22 | 5.00, d (1.6) | 3.27, s |
| 27 | 1.62, s | 1.73, s |
| 28 | 1.24, s | 1.22, s |
| 30 | 2.82, m | 2.82, m |
| | 2.64, m | 2.63, m |
| 31 | 3.74, m | 3.74, m |
| 34 | 8.49, d, (1.6) | 8.49, d (1.6) |
| 39 | 8.25, dd (8.1, 1.6) | 8.24, dd (8.1, 1.6) |
| 40 | 7.38, d (8.1) | 7.38, d (8.1) |
| 42, 48 | 6.86, d (8.7) | 6.86, d (8.7) |
| | 6.83, d (8.7) | 6.82, d (8.7) |
| 43, 49 | 6.86, dd (8.7, 2.2) | 6.86, dd (8.7, 2.2) |
| 45, 51 | 7.16, br s | 7.16, d (2.2) |
| 1-OAc | 2.08, s | 2.09, s |
| 11-OAc | 1.88, s | 1.87, s |
| 12-OAc | 2.07, s | 2.08, s |
| 44,50-OPiv | 1.35, s | 1.35, s |

TABLE VI

| No. | 27 | 9 |
|---|---|---|
| 1 | 4.65, d (5.6) | 4.64, d (5.6) |
| 2 | 3.37, m | 3.38, dd (5.6, 3.8) |
| 3 | 3.30, m | 3.30, m |
| 4 | 2.00, m | 1.98, m |
| | | 1.87, m |
| 5 | 1.98, m | 2.01, m |
| 6 | 4.31, br s | 4.31, br s |
| 7 | 3.67, dd (10.6, 4.3) | 3.69, dd (10.6, 4.3) |
| 8 | 2.06, m | 2.05, m |
| 9 | 2.28, t (11.2) | 2.28, t (11.2) |
| 11 | 5.28, d (11.2) | 5.25, dd (11.2, 2.8) |
| 12 | 5.23, s | 5.15, d (2.8) |
| 14 | 1.98, m | 1.97, m |
| 15 | 4.36, br t (9.3) | 4.27, br t (8.6) |
| 16 | 2.47, br t (12.1) | 2.08, m |
| 17 | 1.82, dd (13.5, 10.2) | 2.09, m |
| 18 | 1.01, s | 0.89, s |
| 19 | 0.98, s | 0.96, s |
| 20 | 0.89, d (7.0) | 1.00, d (7.3) |
| 21 | 2.21, m | 1.55, m |
| 22 | 5.01, s | 3.28, s |
| 27 | 1.62, s | 1.74, s |
| 28 | 1.24, s | 1.24, s |
| 30 | 4.25, d (16.0) | 4.23, d (16.0) |
| | 4.11, d (16.0) | 4.12, d (16.0) |
| 33 | 8.54, s | 8.54, d (1.6) |
| 38 | 8.25, d (8.0) | 8.27, dd (8.1, 1.6) |
| 39 | 7.38, d (8.0) | 7.38, d (8.1) |
| 41, 47 | 6.90, d (8.7) | 6.89, m |
| 42, 48 | 6.87, d (8.7) | 6.89, m |
| 44, 50 | 7.16, br s | 7.16, d (2.2) |
| 1-OAc | 2.10, s | 2.10, s |
| 11-OAc | 1.90, s | 1.90, s |
| 12-OAc | 2.07, s | 2.10, s |
| 43,49-OPiv | 1.35, s | 1.35, s |

TABLE VII

| No. | 10 | 11 |
|---|---|---|
| 1 | 4.69, d (5.6) | 4.69, d (5.6) |
| 2 | 3.33, dd (5.6, 3.8) | 3.40, dd (5.6, 3.8) |
| 3 | 3.26, m | 3.34, m |
| 4 | 2.03, m | 2.11, m |
| 5 | 2.06, m | 2.12, m |
| 6 | 4.60, br s | 4.60, br s |
| 7 | 3.78, d (10.0) | 3.78, dd (10.0, 4.8) |
| 8 | 2.04, m | 2.33, m |
| 9 | 2.37, t (10.9) | 2.32, t (11.3) |
| 11 | 5.25, dd (10.9, 2.8) | 5.30, dd (11.3, 2.9) |
| 12 | 5.16, d (2.8) | 5.16, d (2.9) |
| 14 | 2.04, m | 1.97, t (8.9) |
| 15 | 4.33, dd (10.4, 8.4) | 4.28, t (8.9) |
| 16 | 2.47, dd (13.5, 10.4) | 2.06, m |
| 17 | 1.82, dd (13.5, 10.8) | 2.08, m |
| 18 | 1.01, s | 0.91, s |
| 19 | 1.05, s | 1.08, s |
| 20 | 0.86, d (7.0) | 1.00, d (7.3) |
| 21 | 2.18, m | 1.55, m |
| 22 | 4.99, d (1.7) | 3.28, s |
| 27 | 1.61, s | 1.74, s |
| 28 | 1.25, s | 1.24, s |
| 31 | 8.40, s | 8.36, d (1.6) |
| 36 | 8.14, d (8.0) | 8.11, dd (8.1, 1.6) |
| 37 | 7.37, d (8.0) | 7.36, d (8.1) |
| 39, 45 | 6.92, d (8.7) | 6.90, d (8.7) |
| | | 6.88, d (8.7) |
| 40, 46 | 6.86, dd (8.7, 2.2) | 6.85, dd (8.7, 2.2) |
| 42, 48 | 7.14, d (2.2) | 7.17, d (2.2) |
| 1-OAc | 2.07, s | 2.12, s |
| 11-OAc | 1.87, s | 1.91, s |
| 12-OAc | 2.04, s | 2.12, s |
| 41,47-OPiv | 1.34, s | 1.35, s |

TABLE VIII

| No. | 28 | 12 |
|---|---|---|
| 1 | 4.71, d (5.6) | 4.69, d (5.6) |
| 2 | 3.41, dd (5.6, 3.8) | 3.41, dd (5.6, 3.8) |
| 3 | 3.34, m | 3.35, m |
| 4 | 2.13, m | 2.13, m |
| 5 | 2.12, m | 2.12, m |
| 6 | 4.62, br s | 4.62, br s |
| 7 | 3.78, dd (10.2, 4.7) | 3.79, dd (10.1, 4.7) |
| 8 | 2.38, m | 2.35, m |
| 9 | 2.33, t (11.3) | 2.33, t (11.2) |
| 11 | 5.36, dd (11.3, 2.8) | 5.32, dd (11.2, 2.9) |
| 12 | 5.24, d (2.8) | 5.16, d (2.9) |
| 14 | 2.00, dd (10.1, 8.1) | 1.98, t (8.9) |
| 15 | 4.39, dd (10.4, 8.1) | 4.29, t (8.9) |
| 16 | 2.46, dd (13.5, 10.4) | 2.06, m |
| 17 | 1.84, dd (13.5, 9.7) | 2.08, m |
| 18 | 1.04, s | 0.93, s |
| 19 | 1.12, s | 1.10, s |
| 20 | 0.90, d (7.1) | 1.00, d (7.4) |
| 21 | 2.20, m | 1.55, m |
| 22 | 5.01, d (1.5) | 3.34, s |
| 27 | 1.64, s | 1.75, s |
| 28 | 1.28, s | 1.25, s |
| 31 | 8.40, s | 8.38, s |
| 36 | 8.12, d (8.0) | 8.10, dd (8.0, 1.6) |
| 37 | 7.35, d (8.0) | 7.34, d (8.0) |
| 39, 45 | 6.76, d (8.7) | 6.73, d (8.7) |
| | 6.75, d (8.7) | 6.71, d (8.7) |
| 40, 46 | 6.66, d (8.7) | 6.63, d (8.7) |
| | 6.64, d (8.7) | 6.61, d (8.7) |
| 42, 48 | 6.82, s | 6.79, s |
| 1-OAc | 2.12, s | 2.13, s |
| 11-OAc | 1.92, s | 1.91, s |
| 12-OAc | 2.09, s | 2.12, s |

### 19. Fluorescence Assays

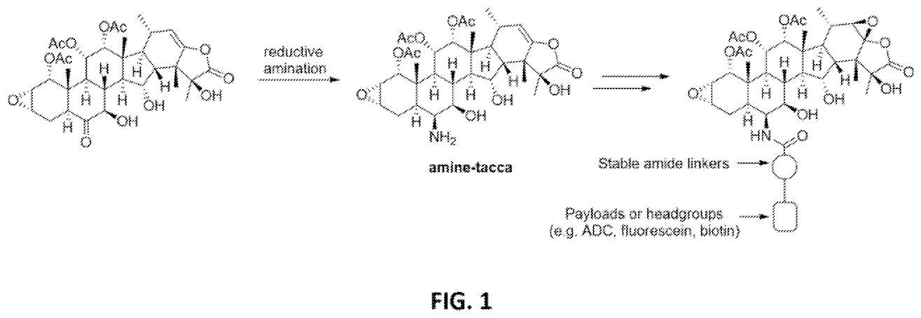

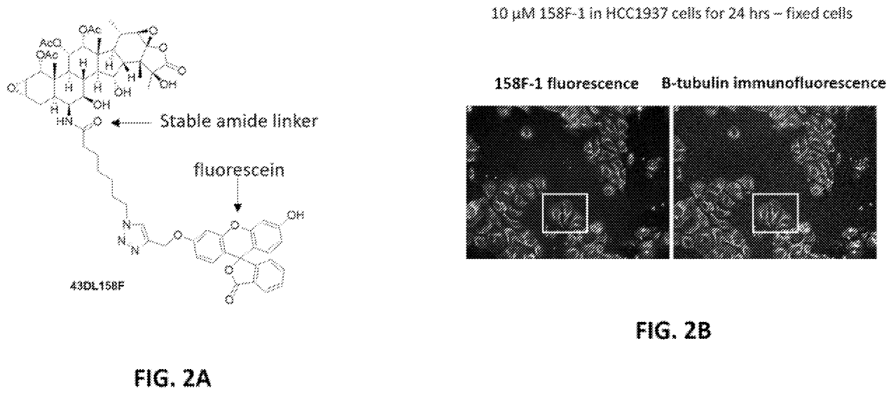

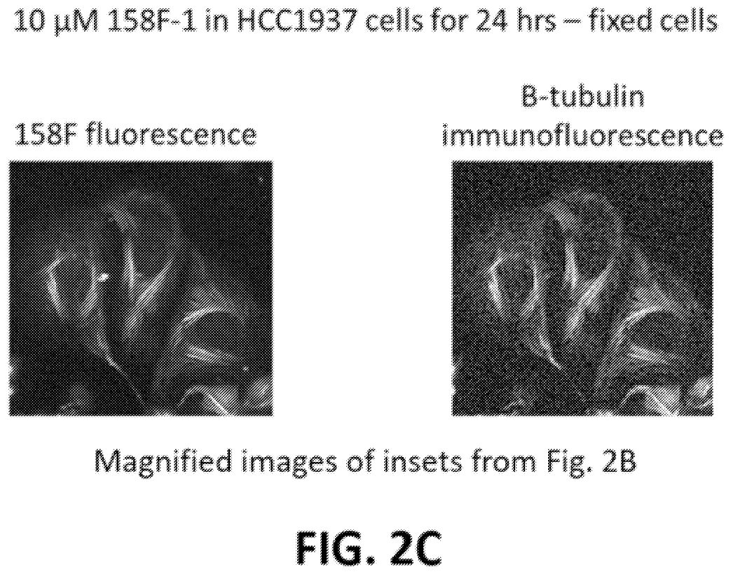

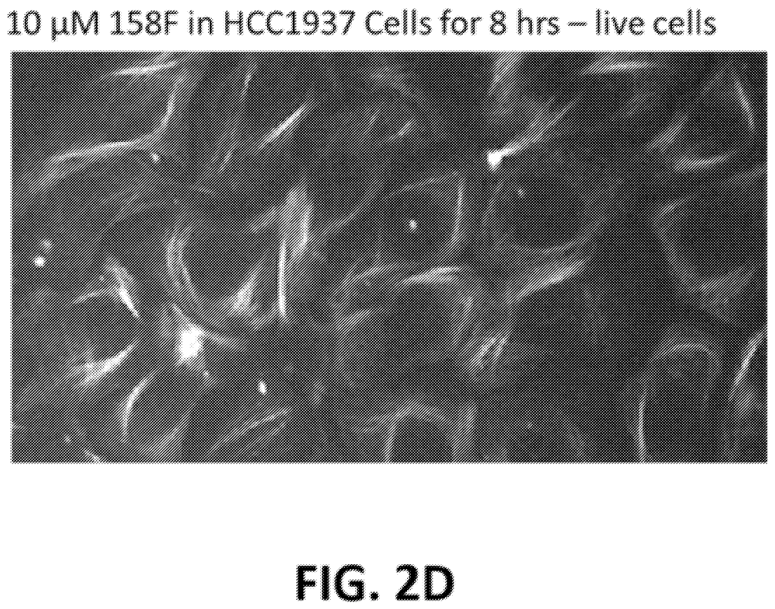

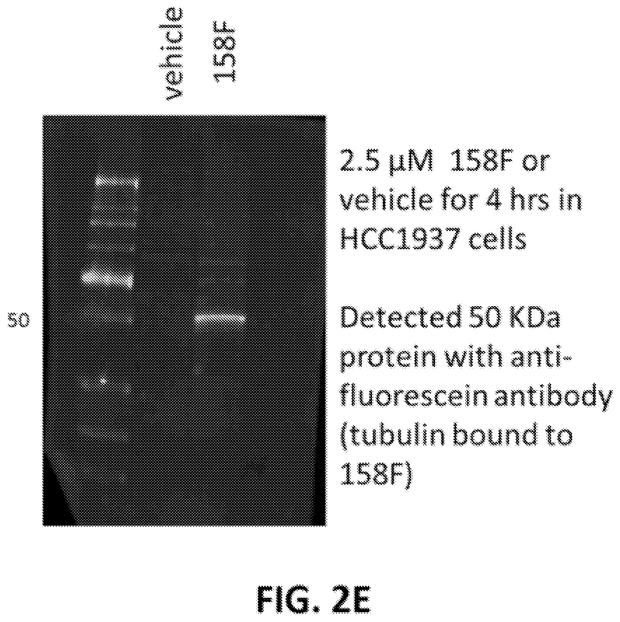

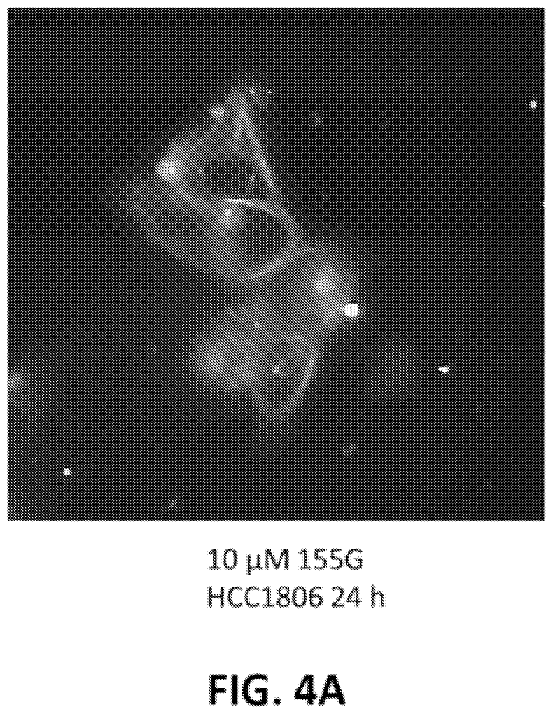

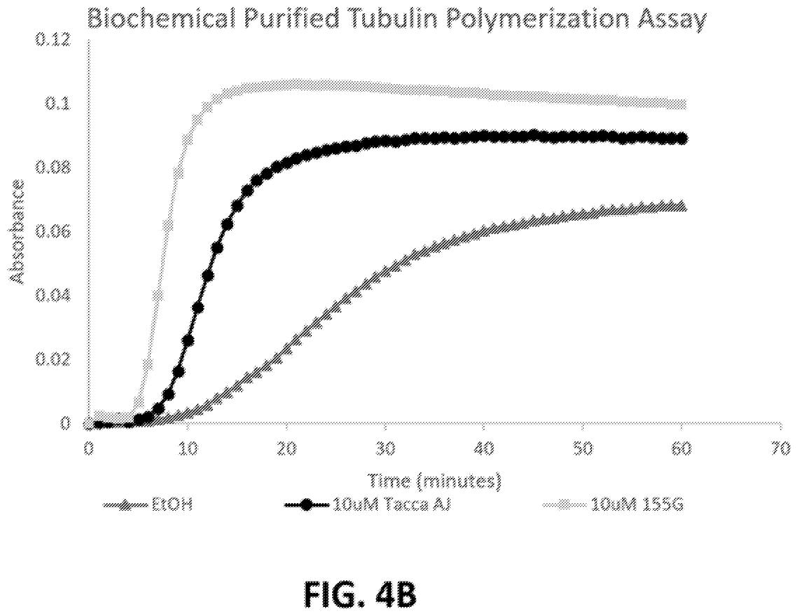

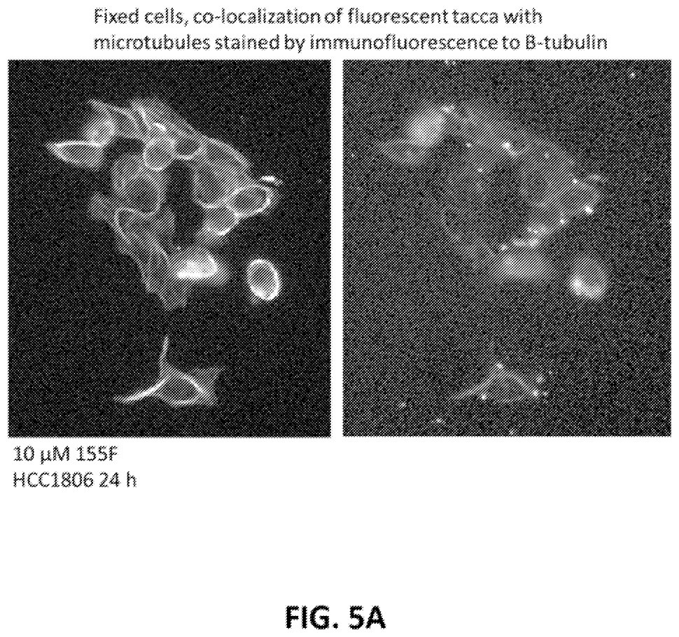

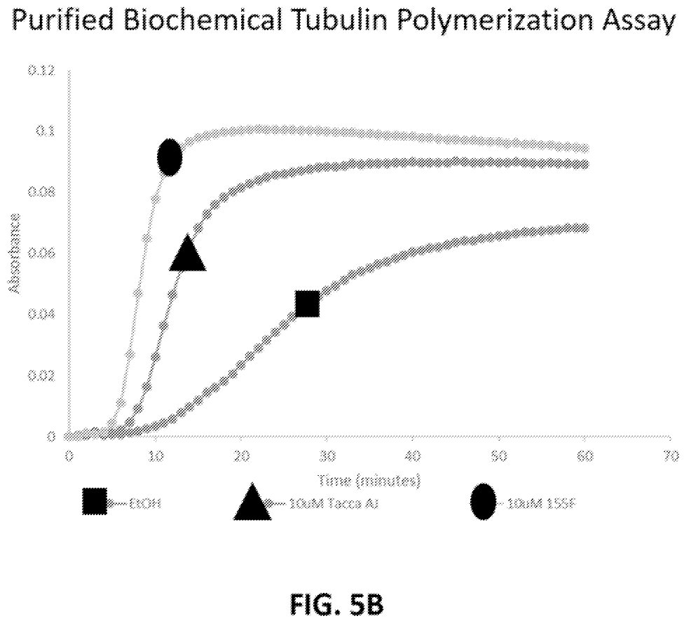

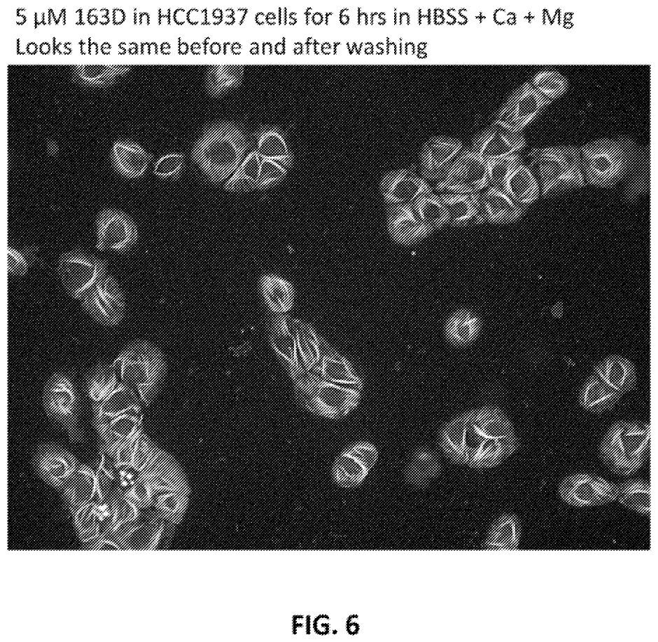

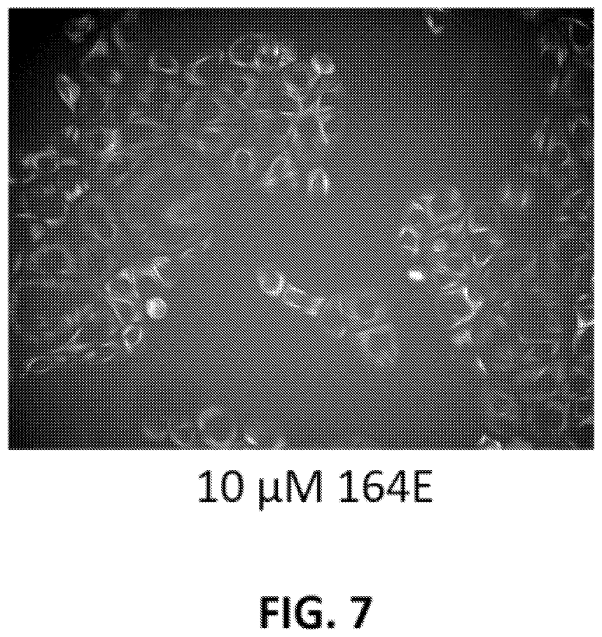

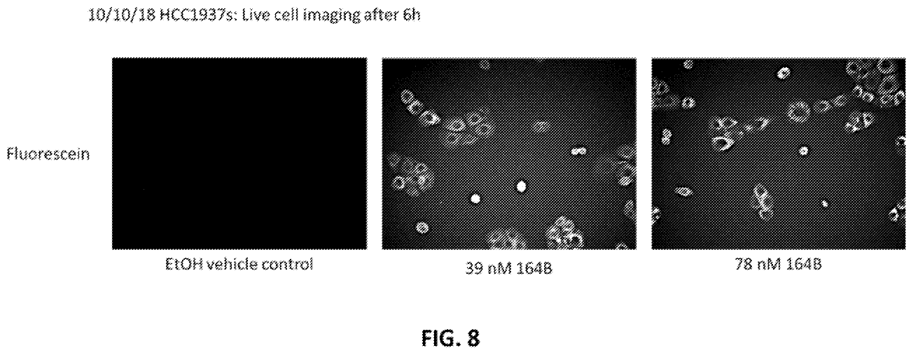

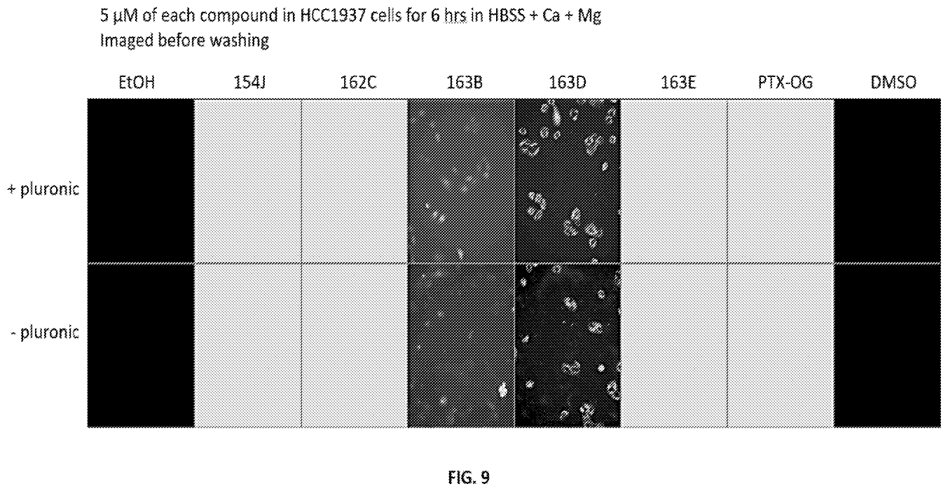

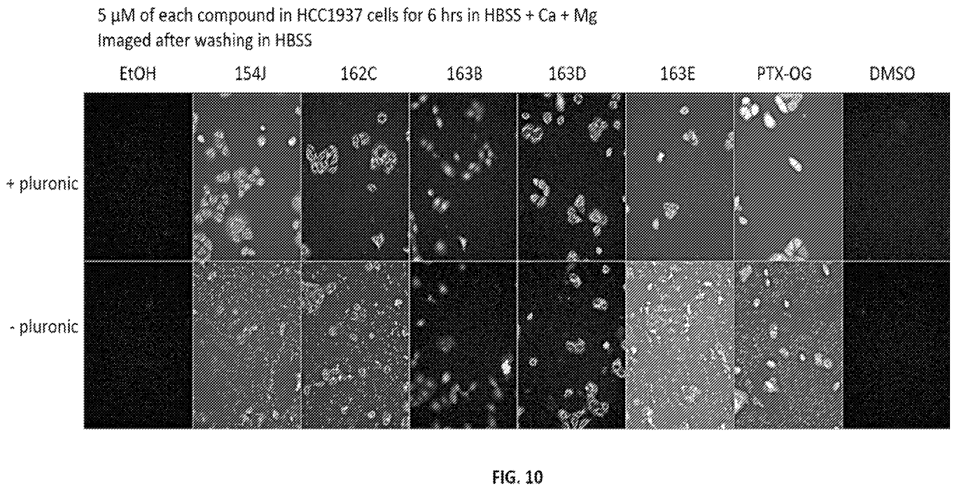

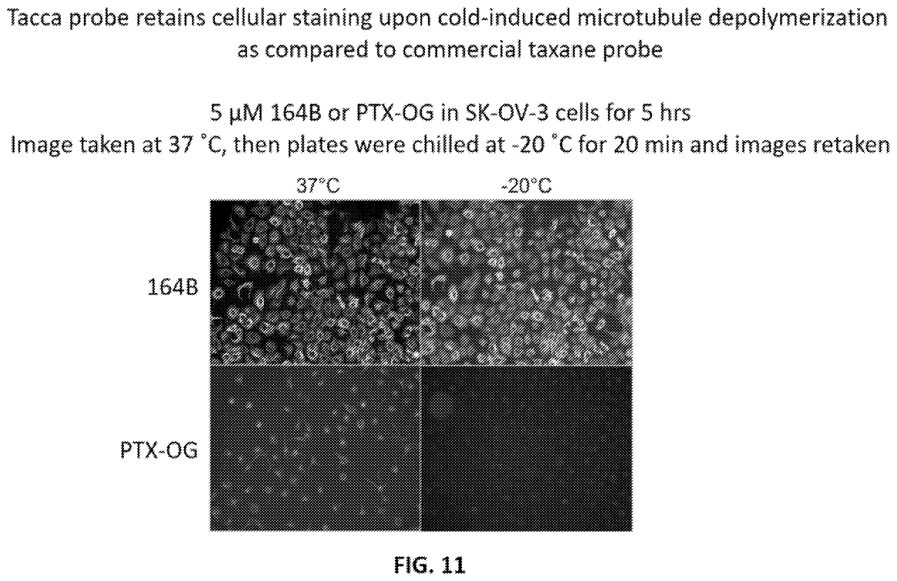

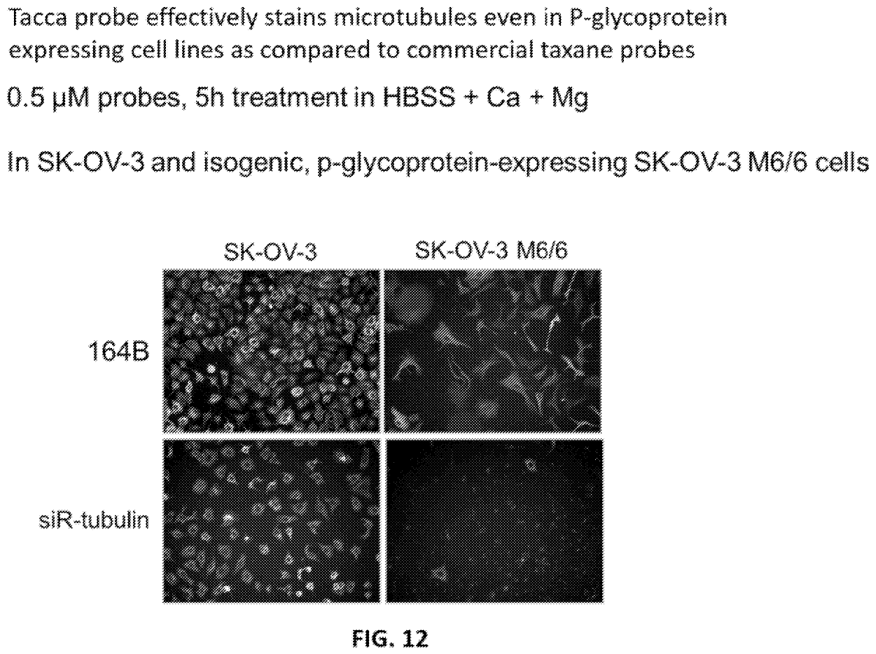

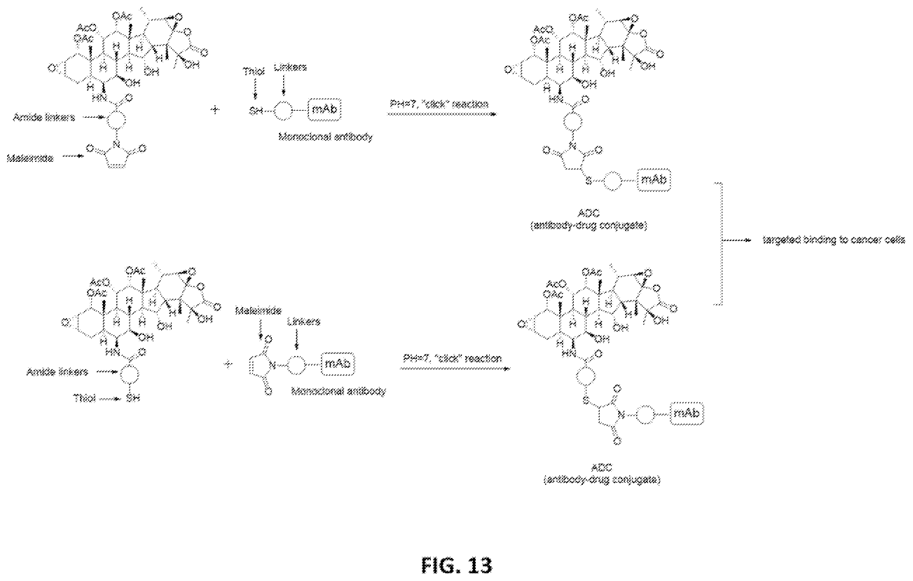

Exemplary results of the fluorescence assays are illustrated in FIG. 2A-E, FIG. 4A, FIG. 5A, and FIG. 6-12. These assays involve direct imaging of the fluorescence of conjugated taccalonolide probes in live cells or in fixed cells in combination with β-tubulin immunofluroecence as described (Du, Risinger, et al., Journal of Natural Products, 2019).

### 20. 51818 Tubulin Polymerization Assays

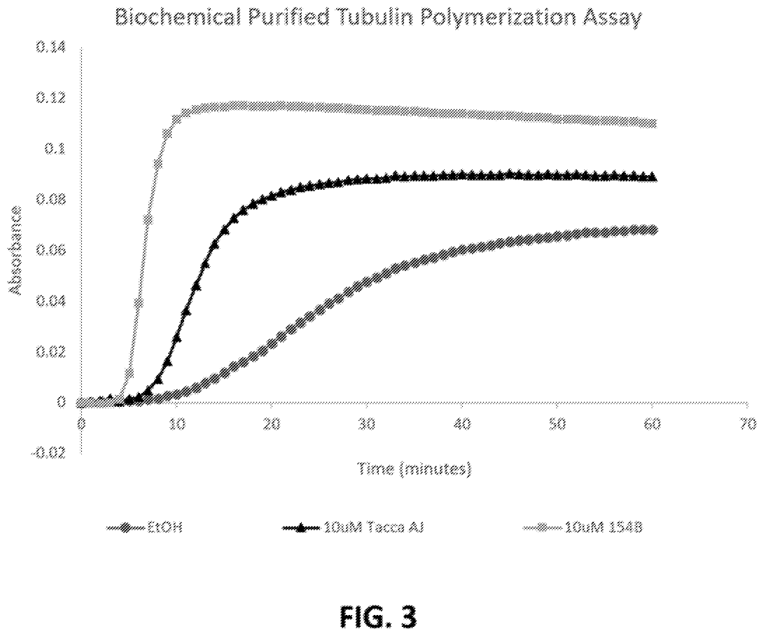

Exemplary results of the 51818 tubulin polymerization assay are illustrated in FIG. 3, FIG. 4B, and FIG. 5B and were obtained as described in Risinger et al., Cancer Research, 2013.

### 21. Computational Modeling

The computational modeling experiments were conducted using the Schrödinger Small-Molecule Drug Discovery Suite (2018-4). The crystal structure 5EZY was downloaded from PDB and optimized using the Protein Preparation Wizard following the standard protocol (Sastry, et al. (2013) J. Comput. Aid. Mol. Des. 27, 221-234). Briefly, the structure was first preprocessed by assigning bond orders, adding hydrogen atoms, creating zero-bond orders to metals, and creating disulfide bonds between two sulfur atoms within 3.2 Å from each other. Prime was used to predict and fill in the missing side chains. Water molecules beyond 5 Å from het groups were removed. To further optimize the model, the H-bond assignment was optimized by sampling water orientations and using PROPKA to assign protonation states of side chains at pH 7.0. All Asp, Glu, Arg, and Lys residues were left in their charged state and the proper His tautomer was also manually selected to maximize hydrogen bonding. Next, a brief relaxation was performed on the structure. This is a two-part procedure that consists of optimizing hydroxyl and thiol torsions in the first stage followed by an all-atom constrained minimization in the second stage to relieve clashes. The minimization was terminated when the RMSD reached a maximum value of 0.30 Å. The optimized protein structure was simplified by only retaining chain B comprising the taccalonolide AJ-β-tubulin complex and removing the other 5 chains. All water molecules were removed except for the one that formed H-bonds between β-tubulin T223 and the 26-carbonyl group of taccalonolide AJ.

The ligand structures were optimized using the Ligand Preparation Wizard (LigPrep) (Sastry, et al. (2013) J. Comput. Aid. Mol. Des. 27, 221-234). Briefly, energy minimization of the ligands was conducted using the OPLS3 force field. The ionization states were generated at pH 7.0 using Epik and the dominating tautomer of each ligand was retained for docking experiments.

Further covalent docking experiments were performed using CovDock (Zhu, K. et al. (2014) J. Chem. Inf. Model. 54, 1932-1940). In the CovDock wizard, D226 was selected as the reactive residue. The docking box was centered on the coordinates X 3.2/Y −63.5/Z 22.6 in the length of 20 Å. The covalent reaction was defined as an epoxide opening reaction that was constrained to take place only on C-22 of the ligands. The “thorough” docking mode was used for the optimization of poses. The cutoff of 2.5 kcal/mol was applied for retaining poses for further optimization in each cycle. The top 10 low-energy poses were generated and retained for each docking experiment. All the 10 docking models were visually checked for the binding interactions of the taccalonolide core structure to filter out the inappropriate binding models with the taccalonolide core structures that were significantly rotated or positioned outside the binding pocket. The lowest-energy pose showing correct spatial arrangement of the taccalonolide core structure was selected for analysis of the ligand-protein binding modes.

### 22. Cell Lines

HCC1806 (CRL-2335) and HCC1937 (CRL-2336) human triple-negative breast cancer cells, HeLa (CCL-2) cervical cancer cells and SK-OV-3 (HTB-77) ovarian cancer cells were obtained from ATCC (Manassas, Va.) and validated by STR profiling (Genetica). SK-OV-3 cells stably overexpressing Pgp by adenoviral-mediated expression of MDR1 were obtained from Dr. Susan Kane and subcloned by limiting dilution to isolate the single-cell clones utilized in these studies as SK-OV-3-MDR-1-6/6 (Risinger, A. L. et al. (2008) Cancer Res. 68, 8881-8888). A single-cell clone from transfection of HeLa cells with βIII-tubulin, designated wild type βIII, was constructed and obtained from Dr. Richard Luduena (Risinger, A. L. et al. (2008) Cancer Res. 68, 8881-8888). HCC1806 and HCC1937 cells were cultured in RPMI 1640 media (Corning) with 10% FBS (Cellgro) and 50 μg/mL gentamicin (Gibco). HeLa, Q111-tubulin expressing HeLa, SK-OV-3 and SK-OV-3/MDR-1-6/6 cells were grown in BME media with Earle's salts (Gibco) with 10% FBS, 1× final 1% GlutaMax™ Supplement (Gibco), and 50 sg/mL gentamicin. The use of HeLa cells allows for the direct comparison of the in vitro potency as compared to other compounds of this class, which have been predominantly reported in this line and compound potencies are consistent among three additional cancer cell lines. HeLa cells were also used for ectopic expression of tubulin mutants due to the high degree of transfectability of this cell line. Cells were tested for mycoplasma contamination using the Mycoplasma Detection Kit-Quick Test (Cat: B39032, Lot: JW004).

### 23. Antiproliferative Assays

The sulforhodamine B (SRB) assay was utilized to examine the antiproliferative and cytotoxic effects of the compounds (Skehan, P. et al. (1990) J. Natl. Cancer Inst. 82, 1107-1112; Vichai and Kirtikara (2006) Nat. Protoc. 1, 1112-1116). Approximately 2,000 cells per well (for SK-OV-3, HeLa and βIII-tubulin expressing HeLa) or 4,000 cells per well (for HCC1806 and HCC1937) were seeded in 96-well plates. For each biological replicate, cells were treated in triplicate with each concentration of compound or vehicle control for 48 h in a final volume of 200 μL. The plates were fixed with 10% trichloroacetic acid for protein precipitation of adherent cells and then washed with distilled water. 100 μL of SRB dye, which binds protein stoichiometrically, was added and then unbound dye removed with 1% acetic acid followed by the addition of 200 μL 10 mM Tris to solubilize the dye, which was quantified by absorbance at 560 nm. The percent growth of treated cells relative to the density at the time of drug addiction was calculated as compared to vehicle treated cells. Concentration-response curves were generated by non-linear regression analysis using Prism software 7.04 (GraphPad) and the GI₅₀ of each compound was calculated and defined as the concentration that caused a 50% decrease in cellular proliferation in the 48 h of drug incubation in comparison to vehicle control from 3 independent experiments.

### 24. Live Cell Fluorescence Imaging and Immunofluorescence

HCC1937, SK-OV-3, and SK-OV-3/MDR-1-6/6 cells were plated in PerkinElmer cell carrier imaging 96-well plates at a density of 8,000-10,000 cells/well. HeLa and HeLa 111-tubulin overexpressing human cervical cancer cells were plated in PerkinElmer cell carrier imaging 96-well plates at a density of 4,000 cells/well. Cells were treated with vehicle control or compounds at the indicated final concentration for each individual experiment. Tubulin Tracker Green and siR-Tubulin stock solutions were prepared in anhydrous DMSO (Sigma Aldrich) at concentrations of 2 mM or 1 mM, respectively. Pluronic® F-127 (Invitrogen) was added at a 1:1 ratio from a 20% (w/v) DMSO stock solution where indicated. For live cell imaging, cells were imaged 5 h after treatment with compound and then washed with fresh media or Hank's Balanced Salt Solution (HBSS) (Sigma-Aldrich) supplemented with 2 mM CaCl₂ and 0.8 mM MgSO₄ and imaged on the Operetta high content imager using Harmony software (PerkinElmer). HeLa and HeLa βIII-tubulin overexpressing human cervical cancer cells were treated with vehicle (ethanol), 0.5 μM or 5 μM of probes respectively for 5 h treatment in HBSS. Wells were washed prior to fixing with methanol. Images were taken with the Operetta at 20×. For co-localization experiments, cells were fixed with methanol after treatment and subjected to immunofluorescence for β-tubulin at 1:1000 (Sigma T-4026) with goat anti-mouse IgG (H+L) cross-absorbed secondary antibody, Texas Red-X at 1:200 (Invitrogen T-862), while the fluorescein-tagged taccalonolide was directly detected. Probe treated SK-OV-3 cells were imaged in medium prior to wash or in PBS after washing or chilling at −20° C. for 20 min and fixed with methanol. For confocal imaging, HCC1937 cells were treated with 0.05-5 μM taccalonolide probes for 6-24 h on glass coverslips in a 6-well plate and fixed with methanol prior to β-tubulin immunofluorescence at 1:1000 (Sigma T-4026) using goat anti-mouse IgG (H+L) cross-absorbed secondary antibody, Texas Red-X at 1:200 (Invitrogen T-862). Confocal images were acquired using a SP8 Leica DMi8 microscope using a 63× oil objective. All images are representative of the phenotypes observed from examining multiple fields from at least 2 independent experiments.

### 25. Tubulin Polymerization Assay

Biochemical tubulin polymerization assays were performed using purified porcine brain tubulin (Cytoskeleton). In individual wells of a 96-well plate, 1 μL of each 100× drug stock was incubated with 20 μM porcine brain tubulin in GPEM glycerol buffer (1 mM GTP, 10% glycerol, 80 mM PIPES pH 6.9, 2 mM MgCl2 and 0.5 mM EGTA) in a final volume of 100 μL. Pure porcine tubulin was prepared on ice at 4° C. to inhibit tubulin polymerization until the assay was initiated, while the plate reader was pre-warmed to 37° C. Tubulin polymerization was measured every minute for an hour by light scattering at 340 nm in a Spectramax plate reader using SoftMax software (Molecular Devices). Light scattering was normalized to the initial measurement for each well. For the probe-tubulin binding assay, samples were prepared on ice in tubes instead of a 96 well plate, and moved to a 37° C. heat block to initiate binding and polymerization. The time zero (0′) sample consisted of tubulin polymerization buffer prior to addition of taccalonolide probe. At each designated time point, 2 μL of the sample was added to 50 ML NuPAGE sample buffer with 20% Q-Mercaptoethanol and 10% of the resulting sample was subjected to PAGE and immunoblotted for β-tubulin at 1:1000 (abeam, ab6046) or fluorescein at 1:500 (abeam, ab19491) with IRDye 680 or 800 goat anti-rabbit secondary antibodies at 1:10,000 (LI-COR Biosciences) and imaged on an Odyssey FC (LI-COR Biosciences).

### 26. Site-Directed Mutagenesis

The QuikChange II XL Site-Directed Mutagenesis Kit (Agilent Technologies) was used according to the manufacturer's directions with any changes noted. The template for mutagenesis was the human TUBB1 ORF mammalian expression plasmid, C-GFPSpark tag from Sino Biological Inc. (HGI 1626-ACG) using the primers listed in Table 1. After Dpn I digestion, amplification products were stored at 4° C. until transformation into DH10B or XL10-Gold competent cells. DNA constructs were isolated using the QIAGEN Plasmid Midi Kit and Thermo Scientific GeneJet Plasmid Mini Kit. DNA concentrations were measured using a NanoDrop 2000 (Thermo Fisher Scientific). All constructs were sequenced using GENEWIZ and sequences verified using SnapGene.

TABLE 1

| Primer | | Tm | Nucleotide |
|---|---|---|---|
| Name | Primer sequence (5'→3') | (° C.) | # |
| K19A For | caaccagatcggtgccgcgttctgggaggtgatc (SEQ ID | 78.68 | 34 |
| | NO: 1) | | |
| K19A Rev | gatcacctcccagaacgcggcaccgatctggttg (SEQ ID | 78.68 | 34 |
| | NO: 2) | | |
| L217A For | gatatctgcttccgcactgcgaagctgaccacaccaac (SEQ ID | 80.05 | 38 |
| | NO: 3) | | |
| L217A Rev | gttggtgtggtcagcttcgcagtgcggaagcagatatc (SEQ ID | 80.05 | 38 |
| | NO: 4) | | |
| L219A For | cttccgcactctgaaggcgaccacaccaacctac (SEQ ID | 78.68 | 34 |
| | NO: 5) | | |
| L219A Rev | gtaggttggtgtggtcgccttcagagtgcggaag (SEQ ID NO: 6) | 78.68 | 34 |
| D226A For | caccaacctacggggctctgaaccaccttgt (SEQ ID NO: 7) | 78.98 | 31 |
| D226A Rev | acaaggtggttcagagccccgtaggttggtg (SEQ ID NO: 8) | 78.98 | 31 |
| D226N For | cacaccaacctacgggaatctgaaccaccttgt (SEQ ID NO: 9) | 79.14 | 33 |
| D226N Rev | acaaggtggttcagattcccgtaggttggtgtg (SEQ ID NO: 10) | 79.14 | 33 |
| T223A For | gaagctgaccacaccagcctacggggatct (SEQ ID NO: 11) | 78.9 | 30 |
| T223A Rev | agatccccgtaggctggtgtggtcagcttc (SEQ ID NO: 12) | 78.9 | 30 |
| H229A For | ctacggggatctgaacgcccttgtctcagccacc (SEQ ID | 79.88 | 34 |
| | NO: 13) | | |
| H229A Rev | ggtggctgagacaagggcgttcagatccccgtag (SEQ ID | 79.88 | 34 |
| | NO: 14) | | |
| R278A For | ccctctcaccagcgctggaagccagcag (SEQ ID NO: 15) | 78.07 | 28 |
| R278A Rev | ctgctggcttccagcgctggtgagaggg (SEQ ID NO: 16) | 78.07 | 28 |

### 27. Activity-Based Protein Profiling and Immunoblotting

For cellular binding studies, HeLa cells were seeded to 80-90% confluence in 6-well dishes and transiently transfected with the wild type or mutant tubulin constructs using Lipofectamine 3000 Transfection Reagent (Thermo Fisher Scientific) for 16-18 h. Media with the lipofectamine reagent were removed from the wells and replaced with fresh BME media for approximately 24 h prior to drug treatment. Cells were treated with 1 μM 11, 1 μM taccalonolide AJ or EtOH vehicle for 1-8 h, respectively. Cells were collected by scraping with a cell lifter and lysed with cell extraction buffer (Invitrogen) supplemented with protease inhibitor cocktail (Sigma-Aldrich), 50 mM NaF, 200 μM Na₃VO₄ (Sigma-Aldrich), and 1 mM phenylmethylsulfonyl fluoride (PMSF) (Sigma-Aldrich). Protein concentration was determined by a Coomassie Plus assay kit (Thermo Scientific), equal amounts of protein resolved by SDS-PAGE on NuPage Bolt 10% Bis-Tris gels (Life Technologies), and transferred to Immobilion-FL PVDF membranes (Millipore). Membranes were blocked in Odyssey blocking buffer (LI-COR Biosciences, Lincoln, Nebr., USA) and probed with anti-fluorescein at 1:500 (abcam, ab19491) or β-tubulin 1:1000 (abcam, ab6046) with IRDye 680 or 800 goat anti-rabbit secondary antibodies at 1:10,000 (LI-COR Biosciences, T8660) and imaged on an Odyssey FC (LI-COR Biosciences). βIII-tubulin was detected using a monoclonal antibody produced in mouse (1:400) (Sigma-Aldrich) clone SDL.3D10, ascites fluid.

Revert total protein staining was utilized to demonstrate relative equal total protein for each lysate (LI-COR Biosciences). The relative binding ratio of mutants was calculated as: (fluorescein signalₘᵤₜₐₙₜ/tubulin signalₘᵤₜₐₙₜ)/(fluorescein signal_{wildtype}/tubulin_{wildtype}) and expressed as percent of the wildtype signal for each independent experiment. For imaging studies, wild type or mutant TUBB1-GFP constructs were transfected into HeLa cells (40,000 cells/well in a 96-well plate) using Lipofectamine 3000 for 16-18 h prior to washing and replacing with fresh media. After 7 h recovery, cells were imaged before and after treatment with vehicle or 100 nM taccalonolide AJ for 22 h using the Operetta. Uncropped blots can be found in the source data file.

### 28. Cellular Biotransformation Assays

HCC1937 cells were grown to 90% confluence then treated with vehicle (ethanol), or 1 μM 10 for 8 h in a total volume of 5 mL. The media was harvested while the cell pellet was lysed by dounce homogenization in a hypotonic buffer (1 mM EGTA and 1 mM MgSO4, pH7) after 2 washes with 1×PBS. The cell lysates were extracted by ethyl acetate. The organic layers were dried down and re-dissolved in MeOH for LCMS analysis.

### 29. qRT-PCR

RNA was isolated from SK-OV-3 and SK-OV-3/MDR-1-6/6 ovarian cancer cells by Trizol and chloroform extraction. The RNA pellet was resuspended in nuclease-free water and quantified using a Nanodrop 2000. RNA was converted to cDNA with iScript Reverse Transcription Supermix for RT-qPCR (Bio-Rad) and qRT-PCR was completed using iTaq Universal SYBr Green Supermix (Bio-Rad). Human Pgp primers (Sigma-Aldrich) were generated based on previous reports (He, S. et al. (2010) Int. J. Mol. Sci. 11, 3309-3051) as Pgp1: 5′-AAAGCGACTGAATGTTCAGTGG-3′ (SEQ ID NO:17) and Pgp2: 5′-AATAGATGCCTTTCTGTGCCAG-3′ (SEQ ID NO:18) and specificity was confirmed using NCBI Primer-BLAST. Human GAPDH primers: 5′-GCAAATTCCATGGCACCGT-3′ (SEQ ID NO:19) and 5′-TCGCCCCACTTGATTTTGG-3′ (SEQ ID NO:20). Relative hPgp mRNA transcript levels were evaluated and presented from two biologically independent experiments, each performed in duplicate.

### 30. Statistics

For binding studies, a one-way ANOVA with Tukey's multiple comparison post-hoc test and adjustment for multiple comparisons were used to determine statistical significance between each condition and significance of mutants as compared to wild type depicted in the figure. Exact n values and P values are in Table 2. For qRT-PCR comparing human Pgp (hPgp) expression in SK-OV-3 and SK-OV-3/MDR-1-6/6 cell lines a one-tailed t-test was performed to give a P value of 0.0307.

TABLE 2

| | Adjusted P values for | Adjusted P values for |
|---|---|---|
| | binding to ectopically | binding to endogenous |
| Comparison | expressed tubulin | tubulin |
| WT vs. K19A | <0.0001 | >0.9999 |
| WT vs. L217A | 0.2353 | 0.9986 |
| WT vs. L219A | 0.0002 | 0.9998 |
| WT vs. T223A | 0.0169 | >0.9999 |
| WT vs. D226A | <0.0001 | 0.9155 |
| WT vs. H229A | 0.0043 | >0.9999 |
| WT vs. R278A | 0.9961 | >0.9999 |
| K19A vs. L217A | 0.0264 | 0.9992 |
| K19A vs. L219A | 0.9976 | 0.9999 |
| K19A vs. T223A | 0.2622 | >0.9999 |
| K19A vs. D226A | >0.9999 | 0.9548 |
| K19A vs. H229A | 0.5605 | >0.9999 |
| K19A vs. R278A | <0.0001 | >0.9999 |
| L217A vs. L219A | 0.0973 | >0.9999 |
| L217A vs. T223A | 0.9209 | >0.9999 |
| L217A vs. D226A | 0.0102 | 0.9997 |
| L217A vs. H229A | 0.6462 | 0.9999 |
| L217A vs. R278A | 0.1267 | 0.9993 |
| L219A vs. T223A | 0.6095 | >0.9999 |
| L219A vs. D226A | 0.995 | 0.9982 |
| L219A vs. H229A | 0.9009 | >0.9999 |
| L219A vs. R278A | 0.0002 | >0.9999 |
| T223A vs. D226A | 0.1579 | 0.9938 |
| T223A vs. H229A | 0.999 | >0.9999 |
| T223A vs. R278A | 0.0107 | >0.9999 |
| D226A vs. H229A | 0.4228 | 0.98 |
| D226A vs. R278A | <0.0001 | 0.9583 |
| H229A vs. R278A | 0.0031 | >0.9999 |

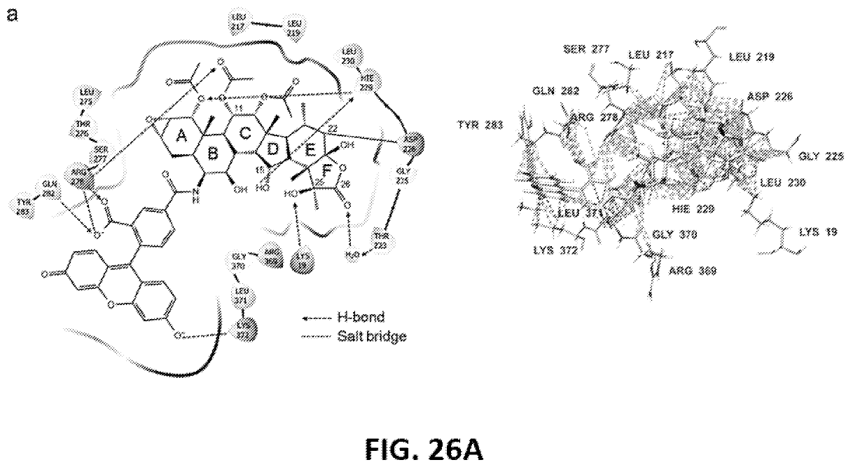

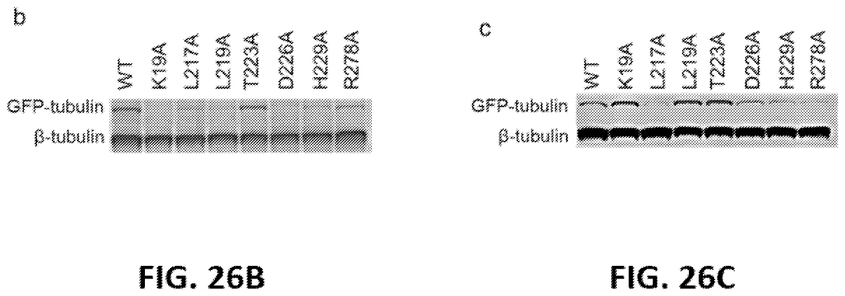

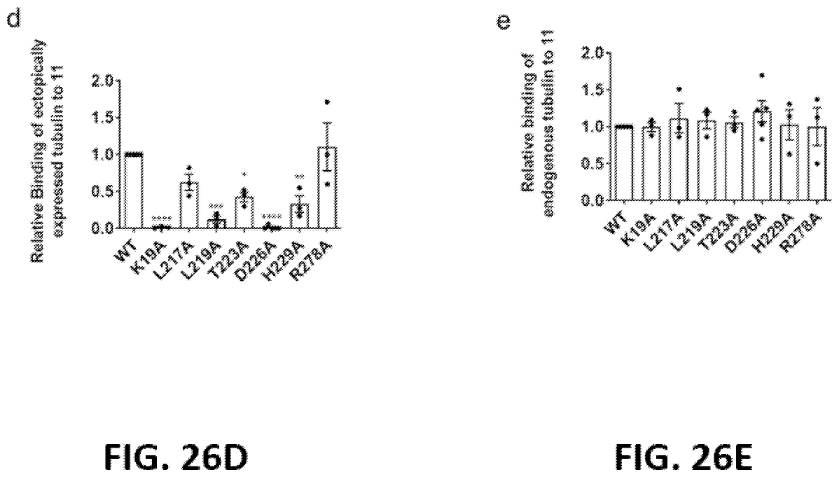

Referring to Table 2, multiply adjusted P values for the binding of 11 to ectopically expressed mutant tubulin (FIG. 26B and FIG. 26D) or endogenously expressed tubulin in the mutant expressing lines (FIG. 26C and FIG. 26E) are shown. Statistical analysis was performed using a one-way ANOVA (20 degrees of freedom) with Tukey's posthoc test. n=3 for all conditions other than WT and D226A controls, where n=5.

Referring to FIG. 26A, the key tubulin residues that mediate the binding affinity of 12 in the docking model structure generated by CovDock. The residues within the radius of 3.5 Å from the probe are displayed in the left graph.

Referring to FIG. 26B and FIG. 26C, HeLa cells were transfected with GFP-tagged TUBB1 constructs with indicated mutations then treated with 1 μM 11 for 8 h. Probe-treated cells were lysed and subjected to immunoblotting. An anti-fluorescein antibody was used to detect 11 bound to endogenous β-tubulin (lower band, 50 kDa) and the GFP-tubulin constructs (upper band, 77 kDa) (FIG. 26B). β-tubulin immunoblotting showed the expression of the GFP-tubulin constructs (upper band, 77 kDa) and endogenous tubulin (lower band, 50 kDa) (FIG. 26C). Referring to FIG. 26D and FIG. 26E, the ratio of ectopically expressed tubulin mutant (FIG. 26D) or endogenously expressed tubulin (FIG. 26E) that is bound to 11 normalized to the ratio of the wild type (WT) form bound to 11. Average±SEM for 2-4 independent experiments. One-way ANOVA and Tukey's post-hoc test were used to calculate statistical significance between each condition. Significance as compared to the R278A mutant that did not impact binding but was not used in data normalization is shown: *p<0.05, **p<0.01, ***p<0.001, ****p<0.0001.

### 31. Optimization of Fluorogenic Taccalonolide-Based Probes

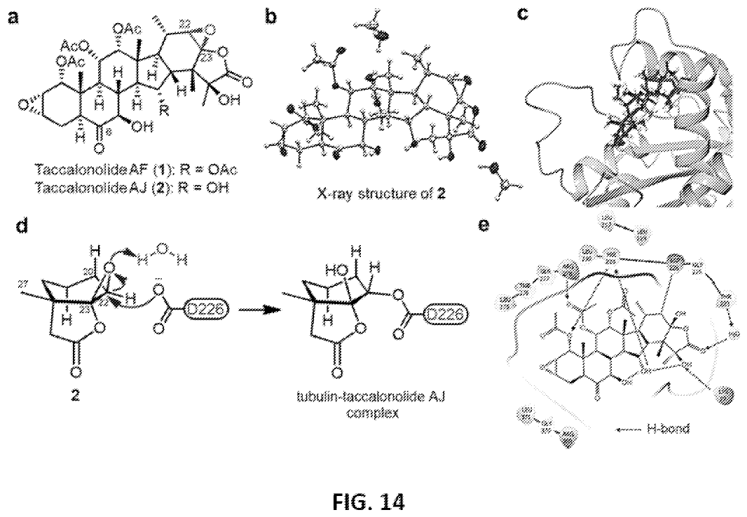

While strategies for generating an optimal taccalonolide-based chemical probe were being explored, Wang et al. reported the crystal structure (PDB ID: 5EZY) of tubulin complexed with taccalonolide AJ (2) (FIG. 14, images (a) and (b)) (Wang, Y. et al. (2017) Nat. Commun. 8, 15787). The authors proposed an unusual reaction mechanism intending to explain the covalent bond formation between the 22,23-epoxy moiety of 2 and β-tubulin D226 residue. However, on account of the existing conflicts in the literature about the absolute configuration of the 22,23-epoxy group (Ola, A. R. B. et al. (2018) J. Nat. Prod. 81, 579-593; Li, J. et al. (2011) J. Am. Chem. Soc. 133, 19064-19067), the 22R,23R configuration of 2 was unambiguously verified by single-crystal X-ray diffraction analysis (FIG. 14, (b)) (Risinger, A. L. et al. (2017) J. Nat. Prod 80, 409-414). Therefore, the opening of the 22,23-epoxy group of 2 is likely facilitated via direct nucleophilic attack by the carboxylate of β-tubulin D226 (FIG. 14, image (d)) (Sanchez-Murcia, et al. (2019) J. Comput. Aided Mol. Des., 33). This epoxide opening mechanism was supported by covalent docking of 2 into 3-tubulin using CovDock affording a lowest-energy docking model that perfectly matched the 5EZY crystal structure (RMSD=0.221, FIG. 14, image (c)) (Zhu, K. et al. (2014) J. Chem. Inf Model. 54, 1932-1940). Analysis of the docking data also disclosed that several other key β-tubulin residues (e.g., K19, H229, R278, L217, L219, and T223) were likely to play important roles in mediating the binding affinity of 2 (FIG. 14, image (e)). But more importantly, the careful examination of the chemical environment in the binding pocket revealed that the C-6 ketone group of 2 was positioned relatively remote from all β-tubulin residues and was not involved in any inter- and intramolecular interactions (FIG. 14, image (e)). Thus, the taccalonolide C-6 position was identified as an optimal site for linker/payload conjugation to generate a stable taccalonolide probe that was likely to maintain the native biological properties of 2.

Referring to FIG. 14, image (a) the structures of taccalonolides AF (1) and AJ (2) showing verified absolute configuration of the 22,23-epoxy moiety are shown. Image (b) shows an ORTEP drawing of the single-crystal X-ray structure of 2 (CCDC ID: 1907790). Image (c) shows the published crystal structure (PDB ID: 5EZY) of 2 (red) bound to β-tubulin superimposed with a model of 2 (blue) docked into β-tubulin (RMSD=0.221) generated by CovDock. Image (d) shows the proposed reaction mechanism between 2 and β-tubulin D226 residue. Image (e) shows the key tubulin residues that mediate the binding affinity of 2 in the docking model structure of c. The residues within a radius of 3.5 Å from the probe are displayed. The key H₂O molecule bridging 2 and T223 was retained as it improved the accuracy of docking experiments.

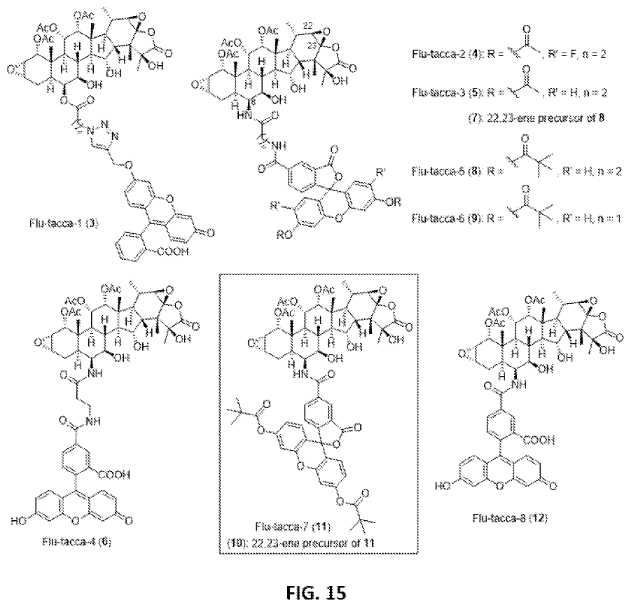

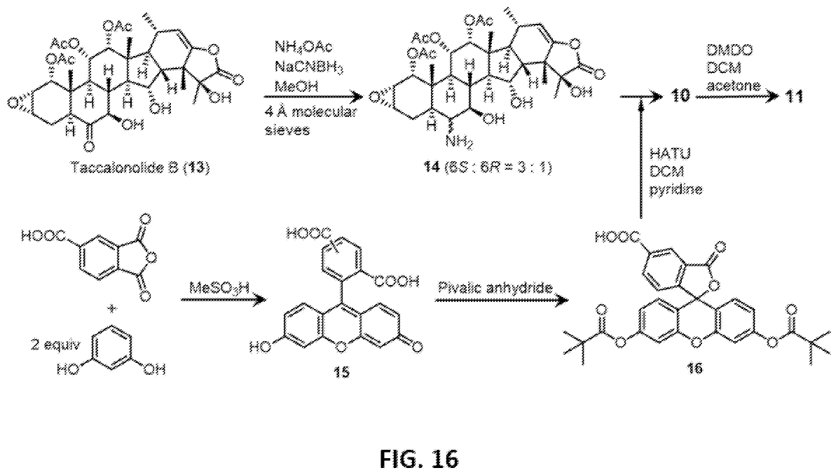

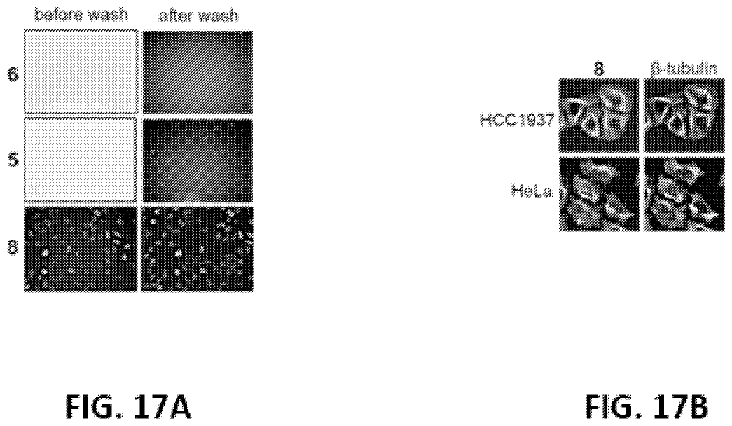

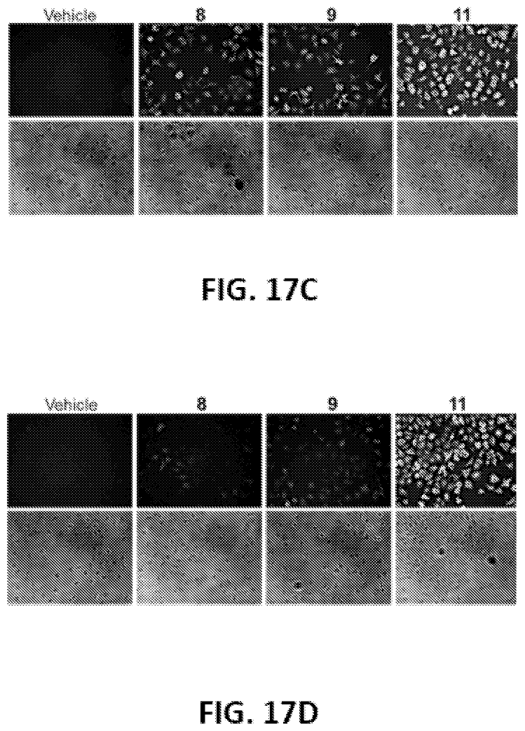

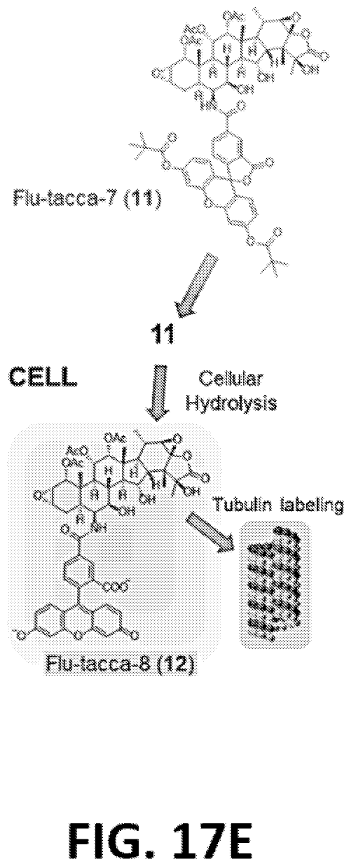

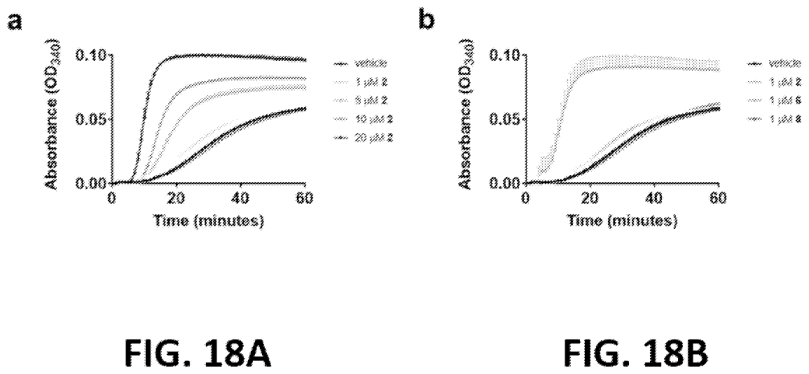

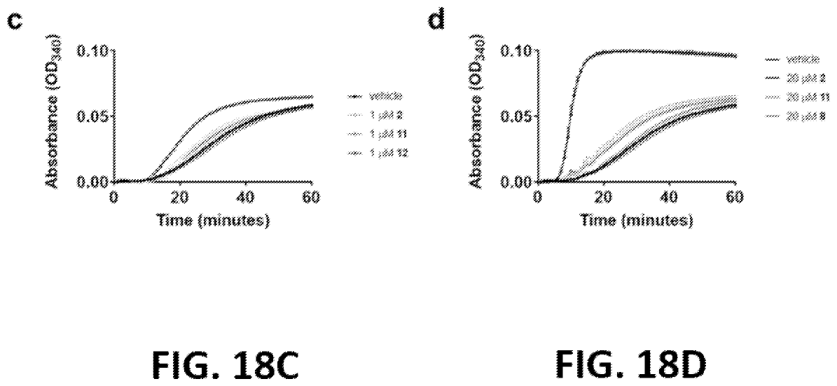

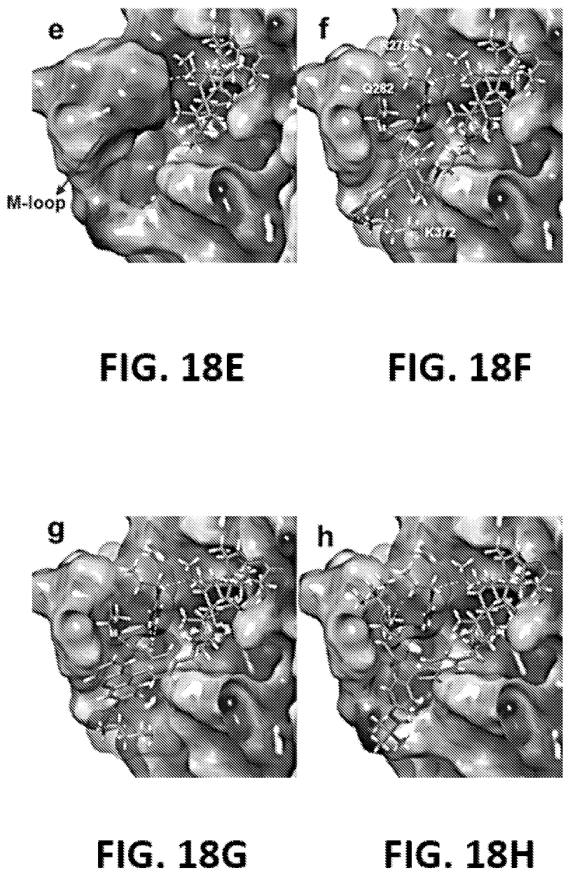

The strategy to functionally characterize taccalonolide-tubulin binding using fluorescent taccalonolide probes is based on the well-established activity-based protein profiling (ABPP) approach (Bottcher, et al. (2010) Angew. Chem. Int. Edit. 49, 2680-2698), which facilitates determination of drug-target interactions in a cellular context and is particularly suited to compounds that covalently bind their targets. Initial attempts to generate a stable taccalonolide probe by modification of taccalonolide C-6 led to the synthesis of Flu-tacca-1 (3) (FIG. 15), a fluorescent probe that enabled direct visualization of the taccalonolides in live cancer cells (Du, L. et al. (2019) J. Nat. Prod. 82, 583-588). However, there were several disadvantages of this probe, including the lability of the ester-based linker, the weak micromolar cellular potency, poor fluorescence properties due to the masked phenolic hydroxyl group of the fluorescein moiety, and high background fluorescence that necessitated removal of excess probe from the media prior to imaging. Without wishing to be bound by theory, these limitations urged for additional taccalonolide probes to be generated that were more suitable for ABPP studies. By means of a survey of various strategies to effectively modify the C-6 position of the taccalonolides, a convenient approach was identified to convert taccalonolide B (13) to its C-6 amino analogue 14 through reductive amination (FIG. 16). The employment of the 4 Å molecular sieve as a dewatering agent in the reaction played a vital role in suppressing the formation of the C-6 hydroxy side product (<5% yield) (Du, L. et al. (2019) J. Nat. Prod. 82, 583-588). With 14 in hand as a key intermediate, a set of stable amide-based fluorescent/fluorogenic probes 4-12 were generated, which employ varying linker length, fluorescent moieties, and prodrug strategies (FIG. 15). The optimization of the taccalonolide probes was guided by evaluation of their biological properties and comparison with the untagged taccalonolide AJ (2) in a series of cellular and biochemical experiments (Table 3, FIG. 17A-E, and FIG. 18A-H).

TABLE 3

| Compound | HeLa | SK-OV-3 |
|---|---|---|
| 2 | 8.5 ± 0.1 | 6.2 ± 1.4 |
| 3 | 2100 ± 200²⁶ | ND |
| 4 | >20000 | >20000 |
| 5 | 12200 ± 1700 | 11800 ± 2100 |
| 6 | 8300 ± 600 | 10800 ± 1600 |
| 7 | >20000 | >20000 |
| 8 | 1500 ± 400 | 4500 ± 900^{a} |
| 9 | 740 ± 60 | 970 ± 60 |
| 10 | >20000 | >20000 |
| 11 | 31 ± 2 | 47 ± 8^{a} |
| 12 | 3000 ± 200^{a} | 7200 ± 400 |

^{a}data from 4 independent experiments

Referring to Table 3, concentrations (nM) of taccalonolide probes that cause a 50% decrease in the proliferation (GI₅₀) of HeLa or SK-OV-3 cells are shown. GI₅₀ values were obtained from three independent experiments (unless otherwise noted) each performed in triplicate and presented as mean±SEM.

Referring to FIG. 17A, visualization of unprotected (6), acetyl-protected (5), and pivaloyl-protected (8) probes in live SK-OV-3 cells 5 h after the addition of 5 μM probe either before (left) or after (right) medium containing probe were removed and replaced by fresh medium are shown. FIG. 17B shows the co-localization of 8 (green) with β-tubulin (orange) immunofluorescence in fixed HCC1937 and HeLa cells after 10 μM probe addition for 6 h. Referring to FIG. 17C and FIG. 17D, pivaloyl-protected taccalonolide-fluorescein probes with decreasing linker sizes (left to right) were added to SK-OV-3 human ovarian cancer cells at a concentration of 5 μM (FIG. 17C) or 0.5 μM (FIG. 17D) and incubated for 5 h prior to imaging. The same image acquisition and processing conditions were used for each image to directly compare relative fluorescence properties (top rows) along with brightfield images of the field (bottom row). Referring to FIG. 17E, a no-wash fluorogenic labeling system for cellular tubulin employing the dipivaloyl-protected taccalonolide probe Flu-tacca-7 (11) is shown.

Referring to FIG. 18A-D, the effects of free taccalonolide AJ (2) and taccalonolide-fluorescein probes (6, 8, 11, 12) on the polymerization of purified porcine tubulin are shown. Specifically, FIG. 18A shows concentration-dependent (1-20 μM) effects of 2 on tubulin polymerization. The deprotected taccalonolide probes (6) (FIG. 18B) and (12) (FIG. 18C) are more potent than 2 in their ability to polymerize purified tubulin, however introduction of pivaloyl protecting groups in 8 and 11 diminish this activity even at equimolar concentrations with tubulin (20 μM, FIG. 18D).

Referring to FIG. 18E-H, a comparison of the published structure 5EZY (2 bound to β-tubulin) (FIG. 18E) with a model of β-tubulin docked to taccalonolide probes (6) (FIG. 18F), (12) (FIG. 18G), and (11) (FIG. 18H) is shown. The selected H-bonds and salt bridges are displayed as yellow and magenta dashed lines, respectively.

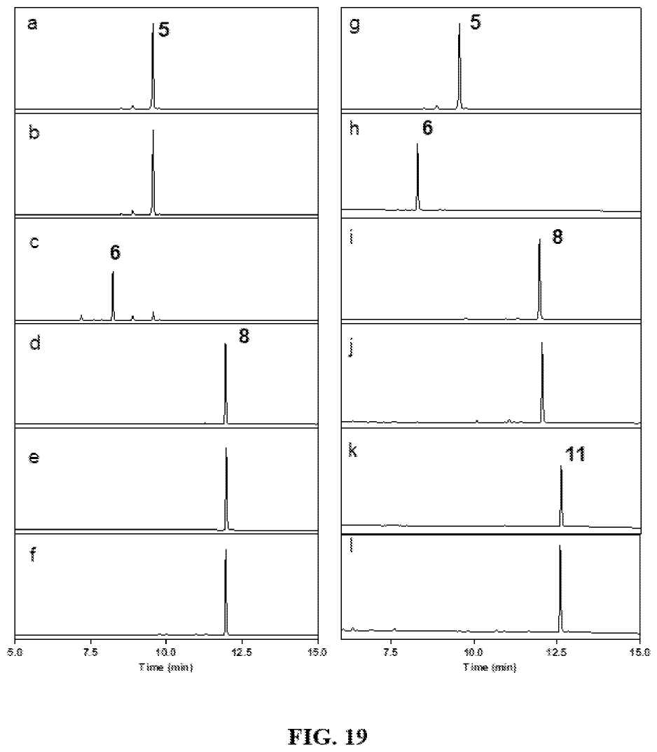

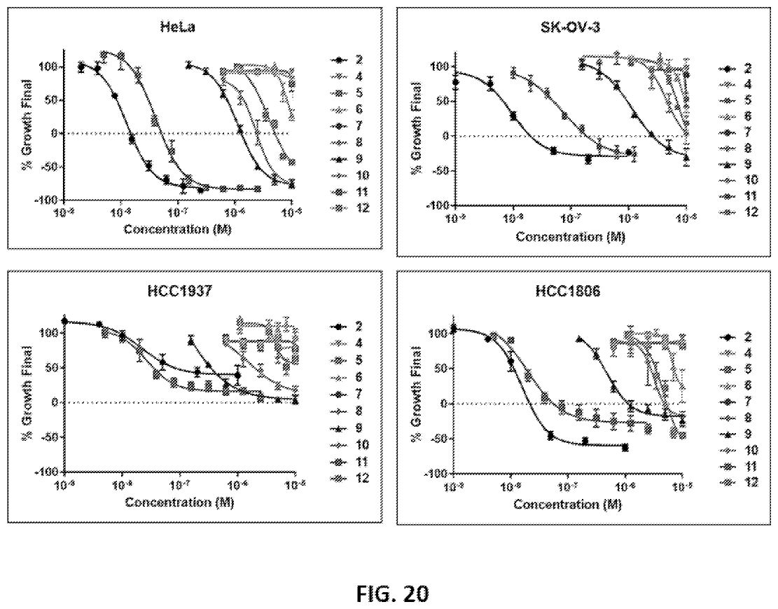

The synthesis of the amide-based probes, Flu-tacca-2 (4) and Flu-tacca-3 (5), was inspired by the structure of the commercial taxane-based probe, Tubulin Tracker Green (Thermo Fisher Scientific, Oregon Green™ 488 Taxol, Bis-Acetate). Specifically, a protected fluorescent moiety (Oregon Green 488 for 4 and fluorescein for 5, diacetyl form) was conjugated with 14 via a β-alanine linker. According to the manufacturer's description (ThermoFisher. www.thermofisher.com/order/catalog/product/T34075?SID=srch-srp-T34075), the diacetyl protection on Oregon Green is intended to quench fluorescence prior to intracellular hydrolysis of the acetyl groups by esterases to decrease background fluorescence of any unincorporated probe. However, the Oregon Green probe 4 was rapidly hydrolyzed even in methanol solutions likely due to the relatively low pKₐ of the Oregon Green moiety (Oregon Green, pKₐ 4.8 and fluorescein, pKₐ 6.5, unprotected form) (Mottram, L. F., et al. (2006) Org. Lett. 8, 581-584) and did not have antiproliferative potency up to the concentration of 20 μM. In contrast, the fluorescein probe 5 showed improved stability in organic solutions but was readily hydrolyzed to yield the deprotected form 6 in a 50% methanol/PBS solution and in the RPMI 1640 medium (FIG. 19, panels (a)-(c), (g), and (h)). Both 5 and 6 were over 125-fold less potent than the untagged 2 against HeLa cervical, SK-OV-3 ovarian, and two triple-negative breast (HCC1806 and HCC1937) human cancer cell lines as determined by the concentration that caused a 50% inhibition of proliferation as compared to vehicle treated controls (GI₅₀) (Table 3 and FIG. 20). Furthermore, both 5 and 6 showed strong extracellular fluorescence in the cell culture medium that remained at a low level even after excess probe was removed from the medium prior to imaging (FIG. 17A). Without wishing to be bound by theory, these results indicated that the diacetyl protection of fluorescein or Oregon Green is not an optimal strategy for generating stable, cell-permeable taccalonolide probes for imaging applications.

Referring to FIG. 19, the MeOH solutions of pure 5 (panel (a)) and 8 (panel (d)) were analyzed by LCMS to provide the controls. The MeOH [panels (b) and (e)] and 50% MeOH/PBS solutions [panels (c) and (f)] of 5 and 8 were kept overnight in static followed by LCMS analysis. Panels (g)-(l) show the hydrolytic stability of 5, 8, and 11 in complete RPMI 1640 medium with 10% FBS. The MeOH solutions of pure 5 (panel (g)), 8 (panel (i)), and 11 (panel (k)) were analyzed by LCMS to provide the controls. Each compound (100 μM) was incubated in 0.1 mL RPMI 1640 medium at 37° C., respectively, for 1 hr (5, panel (h)) or 16 hrs (8, panel (j), and 11, (panel (1)) in static.

Referring to FIG. 20, for each graph, 2 (black circle), 4 (orange upside-down triangle), 5 (brown square), 6 (grey triangle), 7 (purple circle), 8 (green diamond), 9 (dark blue triangle), 10 (light blue diamond), 11 (red square), and 12 (pink square). All points are from 3 biologically independent experiments, each performed in triplicate represented as mean f SEM with the exception of the following: n=4 for 5, 6, 8, 11, and 12 in SK-OV-3, n=2 for 10 in HeLa and HCC1937.

As efficient separation protocols for the synthetic isomeric mixture 5(6)-carboxyfluorescein (15) (FIG. 16) were being explored, it was noticed that the dipivaloyl protection of 15 to give 16 resulted in a complete quenching of carboxyfluorescein fluorescence in aqueous solutions and enabled baseline-separation of the two isomers by HPLC using a preparative C18 column. Intrigued by the quenched fluorescence of 5-carboxyfluorescein dipivalate (16), the taccalonolide probe Flu-tacca-5 (8), a dipivaloyl-protected analogue of 5 and 6 (FIG. 15), was synthesized. As expected, compound 8 exhibited exceptional hydrolytic stability in both organic and aqueous solutions as well as in the cell culture medium (FIG. 19). Moreover, the dipivaloyl protection effectively quenched the fluorescence of the extracellular probe in cell culture media enabling comparable intracellular staining in live cells before and after excess probe was removed from the medium with virtually no background fluorescence (FIG. 17A). Further intracellular localization studies indicated 8 promoted cellular microtubule stabilization and colocalized with β-tubulin immunofluorescence in both fixed HCC1937 and HeLa cell lines (FIG. 17B). Despite the improved visualization of the dipivaloyl probe in both live and fixed cells, the micromolar antiproliferative potency of 8 (HeLa, GI₅₀: 1.5 JIM; SK-OV-3, GI₅₀: 4.5 μM) as compared to the nanomolar potency of untagged 2 (HeLa, GI₅₀: 8.5 nM; SK-OV-3, GI₅₀: 6.2 nM) prompted further optimization of the intracellular potency of dipivaloyl-protected probes.

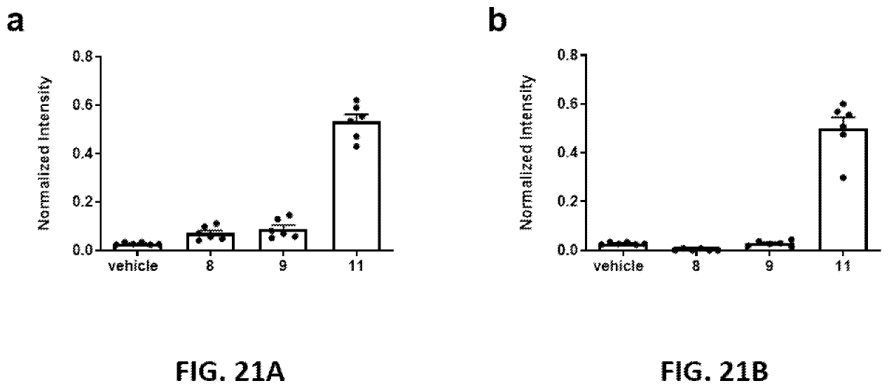

Inspired by a recent publication showing improved antiproliferative activities can be achieved for taxane-based probes by replacing a β-alanine linker with a shorter glycine linker (Lee, et al. (2017) Angew. Chem. Int. Edit. 56, 6927-6931), two dipivaloyl-protected taccalonolide probes were synthesized, including Flu-tacca-6 (9) utilizing a glycine linker and Flu-tacca-7 (11) featuring direct conjugation of the fluorescein moiety with the taccalonolide skeleton by an amide bond (FIG. 15 and FIG. 16). Indeed, a progressive shortening of the linker between the taccalonolide and the dipivaloyl-protected fluorescein moiety enhanced the antiproliferative potency of 9 and 11 as compared to 8 against HeLa and SK-OV-3 cell lines (Table 3). In particular, the direct drug-fluorophore conjugation in 11 led to GI₅₀ values of 30-50 nM, a 50-fold improvement in potency as compared to 8 and less than 10-fold difference as compared to the untagged 2 (Table 3). Furthermore, the potent probe 11 had significantly improved intracellular fluorescence brightness as compared to 8 and 9 when used at equimolar concentrations and imaged under identical acquisition and visualization conditions (FIG. 17C, FIG. 17D, FIG. 21A, and FIG. 21B). Together, these data demonstrate that Flu-tacca-7 (11) represents a cell-permeable, fluorogenic probe that combines the potent antiproliferative activities of taccalonolide AJ with excellent fluorescence properties, including the complete quenching of fluorescence until the dipivaloyl moieties are cleaved, likely by intracellular esterases (FIG. 17E), for the imaging of tubulin in both live and fixed cells.

Referring to FIG. 21A and FIG. 21B, the intensity values from FIG. 4c (FIG. 21A) and 4d (FIG. 21B) were obtained from 6 independent wells, normalized on a log scale to the weakest signal, and presented as mean±SEM.

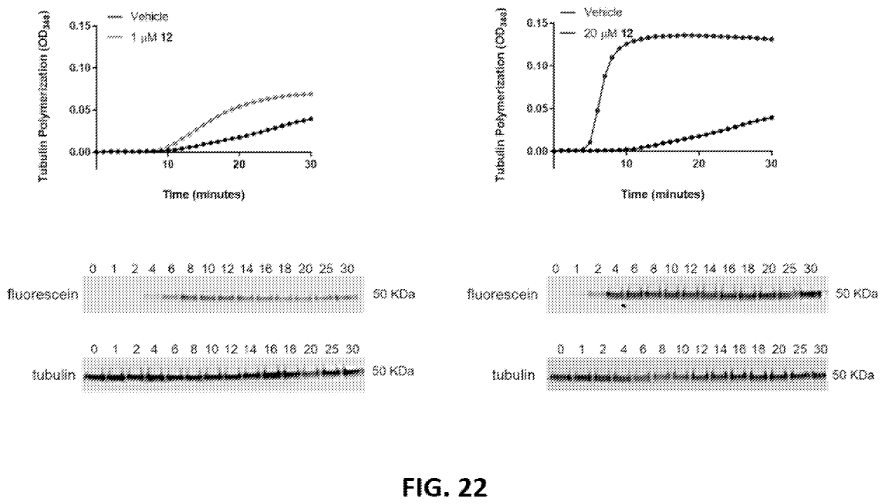

To determine whether the differences in cellular potency among the taccalonolide probes correlated with target engagement, the potency and efficacy of the unprotected probes 6 and 12 and dipivaloyl-protected probes 8 and 11 was evaluated as compared to 2 in a biochemical tubulin polymerization assay. The untagged taccalonolide AJ (2) promoted the polymerization of purified tubulin (20 μM) in a concentration-dependent manner over a range of 5-20 μM (FIG. 18A) as previously described (Risinger, A. L. et al. (2013) Cancer Res. 73, 6780-6792). Unexpectedly, the most potent taccalonolide probe in cellular assays, 11, only slightly enhanced microtubule polymerization at the highest tested concentration (20 μM) (FIG. 18A). In contrast, 12, the deprotected analogue of 11, was more potent than 11 or 2 in this biochemical assay (FIG. 18B). A more dramatic case was observed for the dipivaloyl-protected 8, which did not promote polymerization even at 20 μM, as compared to its deprotected analogue 6, which promoted robust polymerization at 1 μM that was equivalent to the effect of 20 μM of 2 (FIG. 18A and FIG. 18B). Due to the covalent nature of the interaction of the taccalonolide probes with tubulin, it was determined that the time course of binding of 12 to purified tubulin correlated with microtubule polymerization at both 1 and 20 μM (FIG. 22). Without wishing to be bound by theory, this confirms that the lag time associated with tubulin polymerization seen with the taccalonolides (which is distinct from the immediate polymerization observed with the taxanes) is associated with slow initial binding of the drug that then increases rapidly concomitant with microtubule nucleation (Balaguer, F. A. et al. (2019) Int. J. Mol. Sci. 20, E1392).

Referring to FIG. 22, pure tubulin at a concentration of 20 μM was incubated with vehicle (black) or 12 at a concentration of either 1 (top line, left) or 20 μM (top line, right). The extent of microtubule polymerization or 12 binding was determined by the extent of fluorescein signal as compared to tubulin signal by immunoblotting at the indicated times after warming the samples to 37° C. Data are representative of two independent experiments.

Covalent docking using CovDock was employed to rationalize the increased potency of the taccalonolide probes 6 and 12 for biochemical tubulin polymerization as compared to the unmodified taccalonolide 2. The top 10 low-energy poses (ligand-receptor binding models) were generated for 6, 8, 11 and 12, respectively, that were docked into the optimized 5EZY structure. The representative lowest-energy pose obtained from each docking experiment was displayed (FIG. 18C-F). The taccalonolide core structure of 12 (FIG. 18E) was correctly positioned into the taccalonolide binding pocket (based on the 5EZY crystal structure of 2, FIG. 18C) for each of the top 10 low-energy β-tubulin binding models. Interestingly, the fluorescein moiety of 12 occupied an adjacent binding pocket on β-tubulin close to the M-loop affording additional interactions with β-tubulin residues via hydrophobic interactions, H-bonds, and/or salt bridges (FIG. 18E). A similar binding mode was predicted for 6 (FIG. 18D), suggesting that the enhanced ability of 6 and 12 to promote microtubule stabilization in biochemical assays could be attributed to improved binding affinity to β-tubulin afforded by these additional contacts. In contrast, the lowest-energy β-tubulin binding model for 11 clearly showed that the two dipivaloyl protecting groups hampered the ability of the fluorescein moiety to be positioned into this additional binding pocket and the taccalonolide core structure was only correctly positioned in 2 of the 10 low-energy poses (FIG. 18F). In the other 8 low-energy poses for 11, the taccalonolide core structures were “forced” into the binding pocket but the structures were significantly rotated implying those binding models were not reliable and suggesting that 11 was poorly bound to β-tubulin. Similarly, the taccalonolide core structure of 8 failed to correctly fit into the taccalonolide binding pocket in any of the 10 low-energy β-tubulin binding models consistent with the inability of 8 to enhance tubulin polymerization in biochemical assays as compared to vehicle controls (FIG. 18A). Thus, the analysis of taccalonolide probe binding based on covalent docking data was qualitatively consistent with the biochemical tubulin polymerization assay. Although more comprehensive computational and experimental analyses would be required to provide a more complete understanding of the mode of action for the taccalonolide probes, the current binding analysis provides guidance for future efforts to generate taccalonolide analogues with improved binding affinity to tubulin by engaging an additional binding pocket adjacent to M-loop.

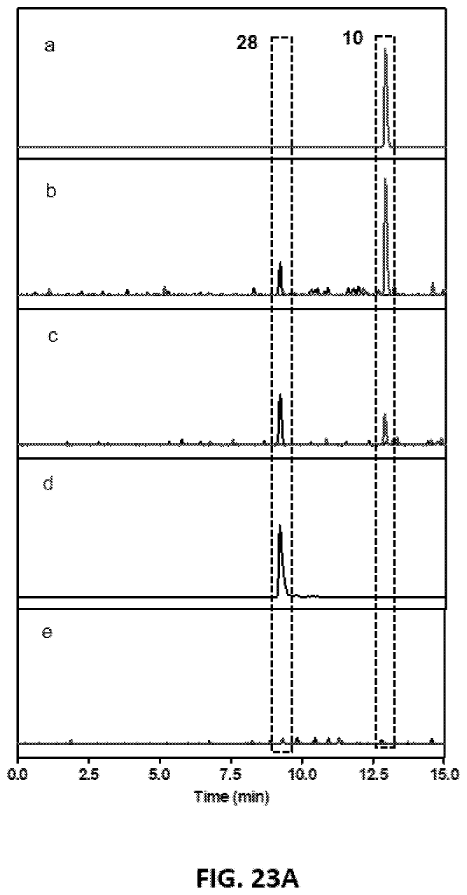

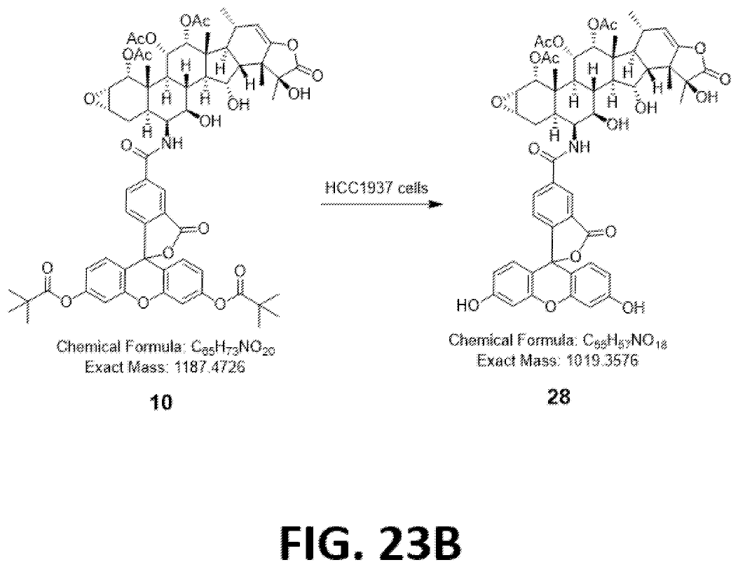

The finding that the dipivaloyl-protected fluorogenic probes 11 and 8 are unable to directly interact with and polymerize tubulin or exhibit fluorescence in the medium, but effectively stabilize microtubules and exhibit fluorescence in cellular assays suggests that upon cellular entry these probes are activated via the hydrolysis of the dipivaloyl groups, likely by cellular esterases, (FIG. 17E). Indeed, when HCC1937 cells were treated with the dipivaloyl protected 22,23-ene analogue (10) of Flu-tacca-7 (11) to prevent irreversible binding to its target, its deprotected, fluorescent form 28 was able to be detected from cellular lysates by LCMS analysis (FIG. 23A, panels (a)-(e) and FIG. 23B). Thus, the dipivaloyl protective groups serve two distinct roles for the taccalonolide probes: effectively quenching extracellular fluorescence and retaining the taccalonolides in a binding-deficient form prior to entry into cells where fluorescence and tubulin binding can both occur.

Referring to FIG. 23A, compound 10 prior to addition to cells (panel (a)) or after addition to HCC1937 cells at 1 μM for 8 h (panel (b)) and (panel (c)), biological replicates, is shown. Left-side trace: negative mode, selected ion at m/z 1018.35 (28); right-side trace: negative mode, selected ion at m/z 1186.46 (10). The LCMS trace of the 28 alone is shown in panel (d) and a control for cells treated with vehicle is in panel (e). Intracellular compounds were detected after the removal of medium and PBS wash 2× prior to lysis, extraction with EtOAc, and re-dissolving the dried extracts in 100 μM MeOH followed by LCMS analysis.

### 32. Target Specificity of the Taccalonolides

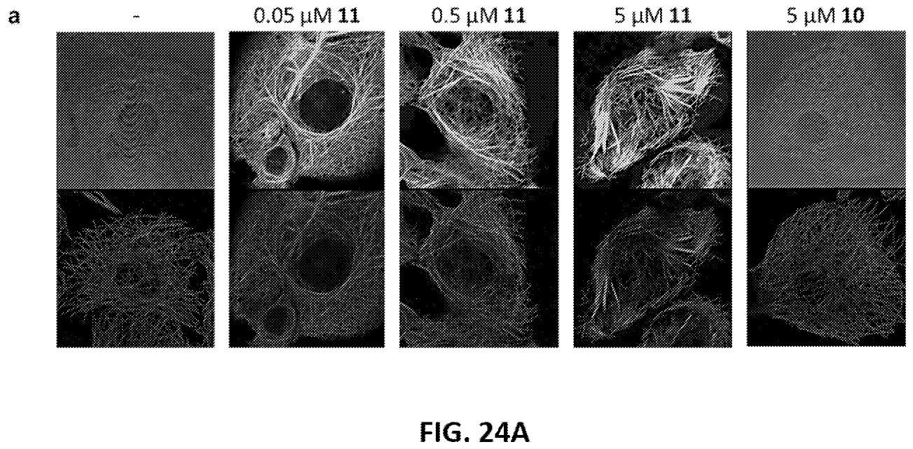

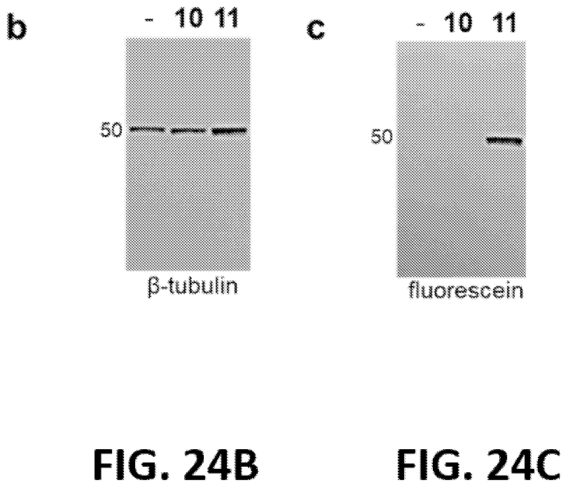

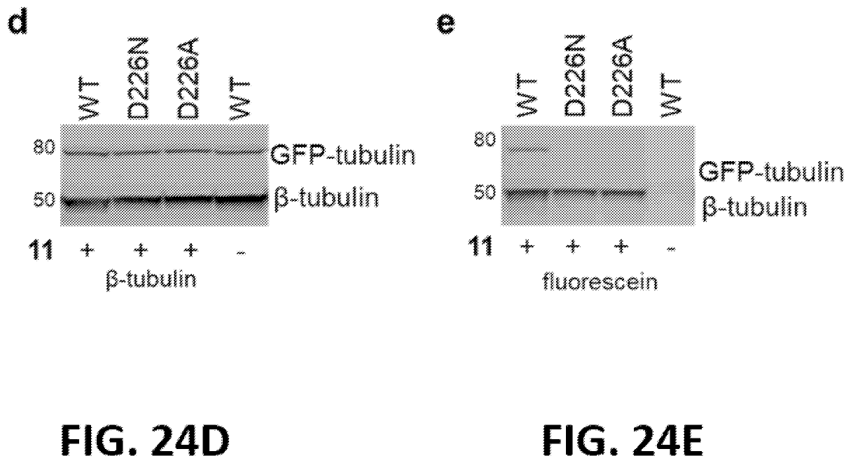

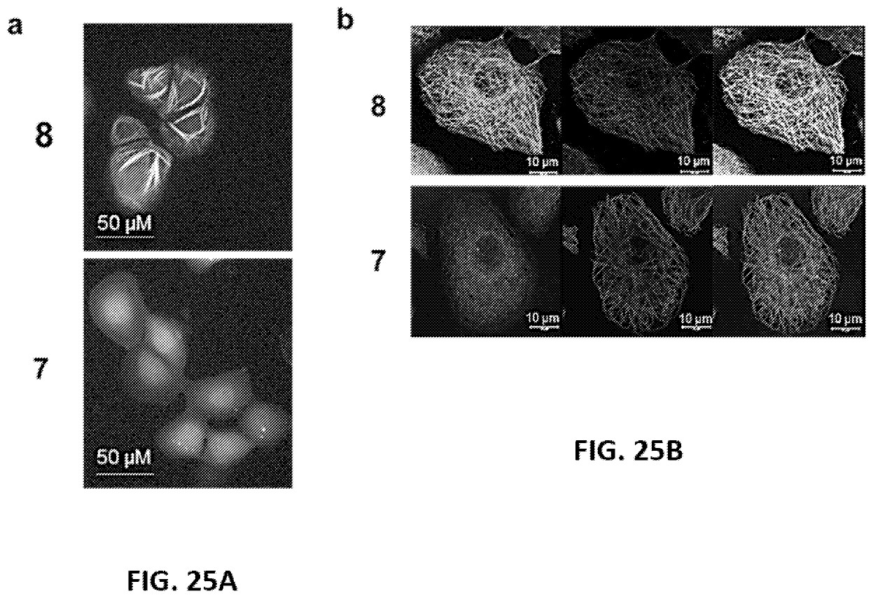

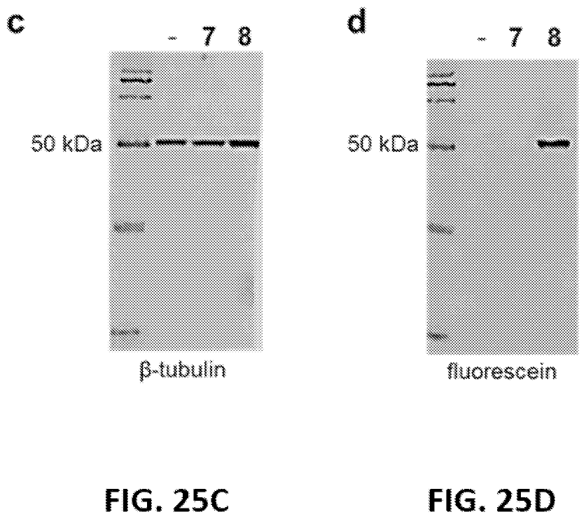

The generation of the potent fluorogenic taccalonolide probe Flu-tacca-7 (11) provides the opportunity to evaluate the covalent binding specificity of the taccalonolides in cell-based assays and fully define the key structural elements of both taccalonolides and β-tubulin that are essential for drug binding. The 22,23-epoxide moiety has been suggested to be critical for the antiproliferative and microtubule stabilizing effects of the taccalonolides (Wang, Y. et al. (2017) Nat. Commun. 8, 15787; Li, J. et al. (2011) J. Am. Chem. Soc. 133, 19064-19067); Peng, J., et al. (2014) J. Med. Chem. 57, 6141-6149). Thus, the antiproliferative activities, cellular localization, and proteome reactivity profiles of Flu-tacca-7 (11) and its 22,23-ene analogue 10 were compared. The single replacement of 22,23-epoxide in 11 by 22,23-ene in 10 completely abrogated the antiproliferative activities in all four human cancer cell lines (i.e., HeLa, SK-OV-3, HCC1806, and HCC1937) up to 20 μM (see Table 3 and FIG. 20). Additionally, 11 promoted a concentration-dependent (0.05 μM-5 μM) increase in the polymerization of cellular tubulin, as detected by immunofluorescence and confocal imaging (FIG. 24A, bottom panels), in line with its antiproliferative potency in HCC1937 cells (FIG. 20). The intrinsic fluorescence of 11 (FIG. 24A, top panels) colocalized completely with the cellular microtubules. In contrast, the 22,23-ene analogue 10 failed to either promote cellular tubulin polymerization (FIG. 24A, bottom panels) or co-localize with microtubules (FIG. 24A, top panels) at 5 μM. This 22,23-epoxide-dependent cellular microtubule stabilization and co-localization was also observed by comparing the localization of 22,23-epoxide and 22,23-ene probes in both live and fixed cells (FIG. 25A and FIG. 25B). On account of the covalent nature of the taccalonolide interaction with tubulin (Risinger, A. L. et al. (2013) Cancer Res. 73, 6780-6792), the protein binding profiles of 10 and 11 were also evaluated by immunoblotting under denaturing and reducing conditions that would disrupt non-covalent interactions using an anti-fluorescein antibody. While the cells treated with 10 or 11 showed equivalent expression of β-tubulin (FIG. 24B), a single 50 kDa fluorescein-containing band was readily identified in cells treated with 11, but not 10 (FIG. 24C). Without wishing to be bound by theory, the results demonstrated that the 22,23-epoxy group was essential for the taccalonolide probes to efficiently form a covalent bond with its major cellular target of β-tubulin in the cellular environment, demonstrating the specificity of this interaction. Similar results were obtained from the comparison of Flu-tacca-5 (8) and its 22,23-ene analogue 7 (FIG. 25C and FIG. 24D). Together, these data strongly demonstrate that the 22,23-epoxy moiety is critical for the covalent reaction of the taccalonolides with β-tubulin.

Referring to FIG. 24A, HCC1937 cells were treated with 0.05-5 μM taccalonolide probes with (11) or without (10) the 22,23-epoxide for 24 h. Confocal imaging-based co-localization of taccalonolide probes (green) with β-tubulin immunofluorescence (red).

Referring to FIG. 24B and FIG. 24C, HCC1937 cells treated with 5 μM 10 or 11 for 6 h were lysed and subjected to immunoblotting using an anti-β-tubulin antibody (FIG. 24B) or an anti-fluorescein antibody (FIG. 24C).

Referring to FIG. 24D and FIG. 24E, HeLa cells were transfected with GFP-tagged TUBB1 constructs with indicated mutations then treated with 1 μM 11 for 8 h. Cell lysates were harvested for immunoblotting. β-tubulin immunoblotting (FIG. 24D) showed the expression of endogenous tubulin (50 kDa) and the GFP-tubulin constructs (77 kDa). In FIG. 24E, an anti-fluorescein antibody was used to detect 11 bound to endogenously expressed β-tubulin (lower band, 50 kDa) and the GFP-tubulin constructs (upper band, 77 kDa).

Referring to FIG. 25A, the visualization of taccalonolide probes 8 and 7 in live HCC1937 cells is shown.

Referring to FIG. 25B, confocal imaging-based co-localization of taccalonolide probe 8 (green) with β-tubulin (red) immunofluorescence in fixed HCC1937 cells as compared to 7.

Referring to FIG. 25C and FIG. 25D, HCC1937 cells treated with 7 or 8 were lysed and subjected to immunoblotting using an anti-β-tubulin antibody (FIG. 25C) or an anti-fluorescein antibody (FIG. 25D).

Inspired by the ability to detect the cellular binding of Flu-tacca-7 (11) to endogenous β-tubulin by immunoblot, a system to evaluate the relative contribution of individual β-tubulin amino acid residues to taccalonolide binding was engineered by performing site-directed mutagenesis on an ectopically expressed β-tubulin construct tagged with GFP at the C-terminus to distinguish it by size on an immunoblot. In order to test the feasibility of this approach, the D226 residue of β-tubulin was first mutagenized to either an asparagine or alanine (see Table 1). HeLa cells that expressed wild type or mutant GFP-tubulin constructs were treated with the probe 11 at 1 μM for 8 h followed by immunoblotting using anti-β-tubulin and anti-fluorescein antibodies. In the β-tubulin immunoblot, bands were detected for each of the GFP-tagged form of β-tubulin (wild type, D226N, or D226A) at 77 kDa that were distinct from endogenous β-tubulin (50 kDa) (FIG. 24D). A fluorescein immunoblot of these same samples demonstrated that 11 was able to interact with the ectopically expressed wild type form of GFP-tubulin, but not either D226 mutant, although it interacted with the endogenously expressed tubulin for all cases as an internal control (FIG. 24E). These results confirm the critical role of the 22,23-epoxide and β-tubulin D226 in taccalonolide-tubulin binding (Wang, Y. et al. (2017) AJ. Nat. Commun. 8, 15787).

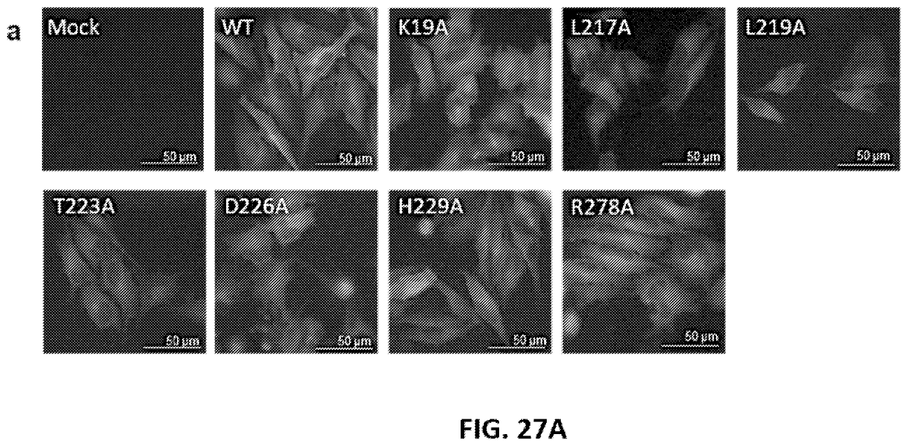

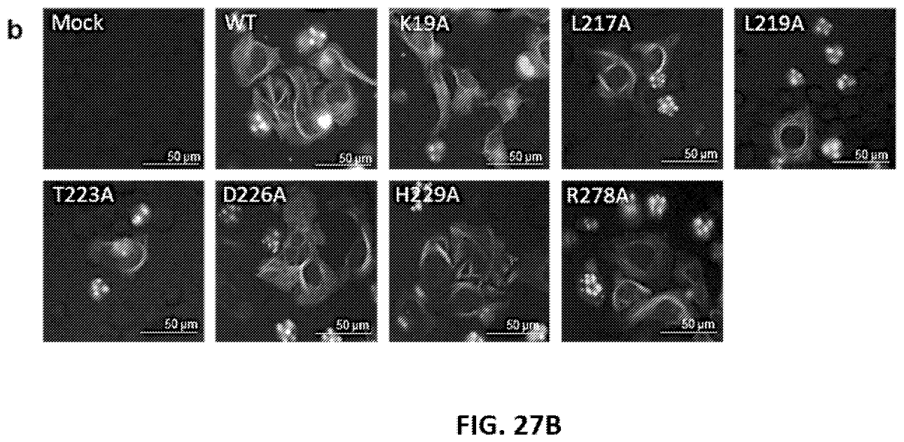

Encouraged by the results of the D226 mutagenesis, additional β-tubulin mutants were constructed in a similar fashion. The analysis of the 5EZY crystal structure (Wang, Y. et al. (2017) AJ. Nat. Commun. 8, 15787) and the covalent docking models of both 2 (FIG. 14, image (e)) and 12 (FIG. 26A) revealed a potential for interaction of the taccalonolide skeleton with 6 residues via H-bonds and/or salt bridges and hydrophobic interactions (i.e., H229, R278, K19, Q282, T223, and K372) and 9 additional β-tubulin residues via only hydrophobic interactions (i.e., L217, R369, L219, L230, L371, Y283, G225, G370, and S277). Overall, the prediction of key interacting residues based on covalent docking to the 5EZY crystallographic structure qualitatively matched the molecular dynamics simulation results of 2 using the cryo-EM structure of a mammalian microtubule (Sanchez-Murcia, et al. (2019) J. Comput. Aided Mol. Des., 33). Thus, besides D226, 6 β-tubulin residues (i.e. K19, H229, R278, L217, L219, and T223) were selected for mutagenesis with similar procedures as described above (see Table 1) followed by immunoblotting to determine their impact on taccalonolide binding. It is important to note that although some of these mutants were expressed at lower levels than wild type GFP-tubulin (FIG. 26B), they were each able to be incorporated into microtubules that were effectively stabilized by the addition of untagged taccalonolide AJ as determined by visualization of the GFP-tag (FIG. 27A and FIG. 27B). The binding ratio of the probe 11 to each GFP-tubulin mutant or the endogenous β-tubulin was determined by quantification and a ratio of fluorescein signal (FIG. 26B) compared to the β-tubulin signal (FIG. 26C) for each band. The binding ratio for each mutant was normalized to that of wild type β-tubulin to determine the relative significance of each tested β-tubulin residue for probe binding to β-tubulin (FIG. 26D).

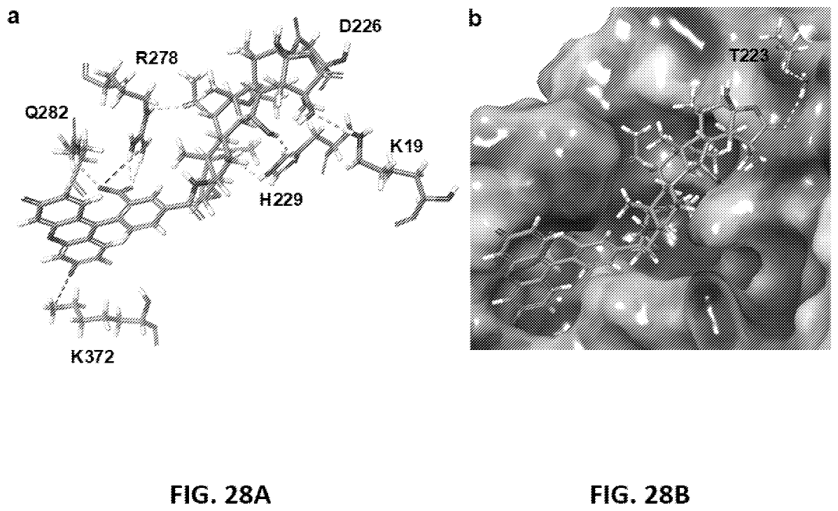

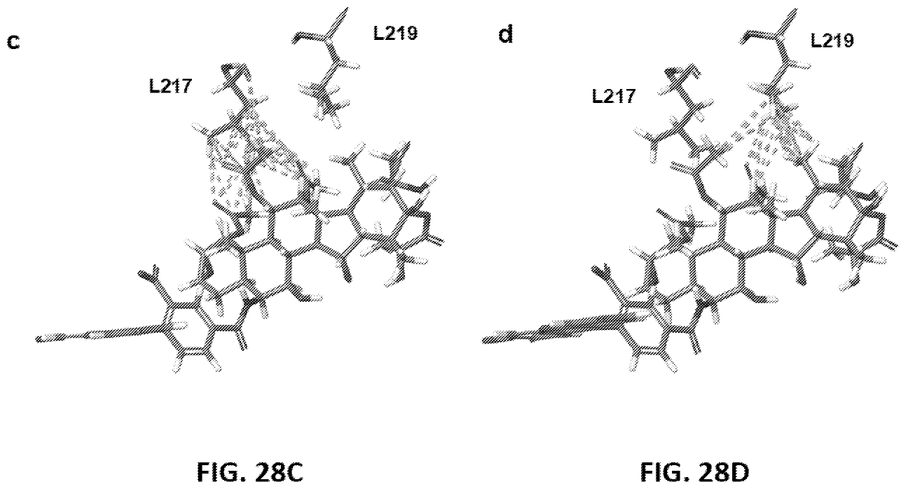

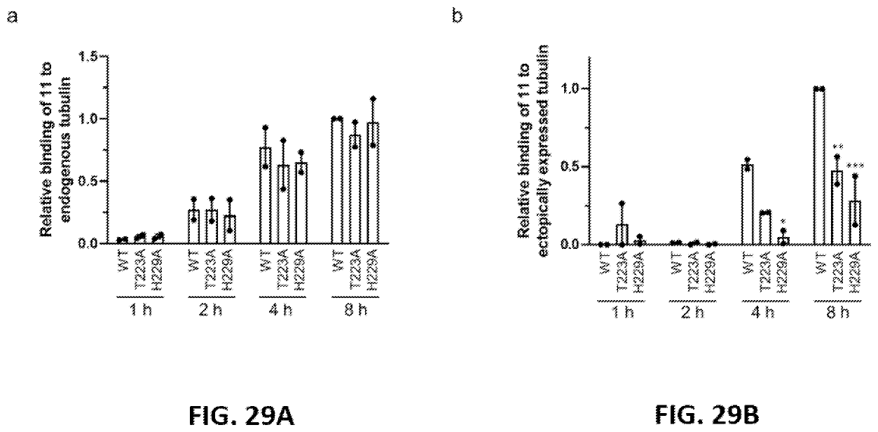

For all of the samples, the probe bound to the endogenous β-tubulin at a similar level (FIG. 26E) indicating the mutagenic manipulations did not affect the extent of the intrinsic binding of the taccalonolide to β-tubulin. Further analysis of the relative binding ratios for GFP-tubulin (FIG. 26D) clearly showed that the mutation of different β-tubulin residues distinctly affected the binding of the probe to β-tubulin. The extent by which different β-tubulin residues affected taccalonolide binding correlated with the type of interaction (FIG. 26Aa and FIG. 28A-D) as well as the distance of the residue to the covalent binding site (C-22 of 11) (Table 4). Specifically, the β-tubulin residues K19, H229, and R278 were predicted to form both H-bonds and hydrophobic interactions with specific moieties on the probe (FIG. 26A), which were progressively distanced from the site of covalent binding (C-22 on probe ring E) (FIG. 28A and Table 4). The K19 side chain strongly interacted with several moieties on ring F (i.e., 25-OH, 26-CO, and 28-Me) which were all close to the covalent binding site (shortest distance, 3.3 Å from C-26 to C-22) (FIG. 26A). Accordingly, the K19A mutation almost completely abrogated the probe binding to a similar extent as the D226 mutations (FIG. 24E, FIG. 26B, and FIG. 26D). In contrast, the H229 side chain mainly interacted with the moieties on rings B-D which were relatively remote from the covalent binding site (shortest distance, 15-OH, 5.2 Å to C-22) (FIG. 26A). Thus, the H229A mutation only moderately inhibited binding (FIG. 26B and FIG. 26D). This observation was consistent with previous SAR studies demonstrating that 25-OH esterification of the 22,23-epoxy taccalonolides dramatically suppressed antiproliferative potency while modification of 15-OH only slightly affected potency (Ola, A. R. B. et al. (2018) J. Nat. Prod. 81, 579-593). Although R278 was predicted to interact with both the taccalonolide core structure (e.g., 11-Me on ring B and 11-Ac on ring C) and the fluorescein moiety via hydrophobic interactions, two H-bonds, and a salt bridge (FIG. 26A), the R278A mutation did not affect probe binding most likely due to the remote location of the interacting sites from the covalent binding site (e.g., 11-Ac, 8.8 Å to C-22). A similar trend was observed for L217 and L219, which were predicted to interact with the taccalonolide core structure via hydrophobic interactions (FIG. 28C and FIG. 28D). Although both L217 and L219 strongly interacted with 11-Ac and 12-Ac on taccalonolide ring C, only L219 showed hydrophobic interactions with 21-Me that collocated with the binding site on ring E. Therefore, the L219A mutation significantly suppressed the probe binding, while the L217A mutation only exhibited a weak inhibitory effect (FIG. 26A and FIG. 26D). An exception was observed for T223 which was predicted to interact with 26-CO on ring F through a H-bond-connected H₂O bridge in the 5EZY crystal structure (FIG. 14, image (e) and FIG. 26A). The T223 hydroxyl was also predicted to play an important role in fixing the carboxylate of D226 to facilitate the covalent reaction with 22,23-epoxide on the basis of molecular dynamics simulation of 2 (Sanchez-Murcia, et al. (2019) J. Comput. Aided. Mol. Des., 33). Interestingly, the T223A mutation only had a moderate effect on binding (FIG. 26A and FIG. 26D) despite the predicted importance of this residue for taccalonolide binding. To gain additional insight into the impact of the T223A and H229A mutants on the rate of taccalonolide binding, the binding of the probe 11 was evaluated at 1, 2, 4 and 8 h. The extent of binding of 11 to endogenous tubulin was minimal at 1 h with increased binding observed at 2-4 h regardless of what form of tubulin (mutant or wild type) was ectopically expressed (FIG. 29A and FIG. 29B). Binding to ectopic wild type tubulin was detectable at 4-8 h with the T223A and H229A mutants showing delays in the rate and extent of binding with a more pronounced effect for the H229A mutant, consistent with FIG. 26A-E (FIG. 29A and FIG. 29B).

TABLE 4

| | | | | Probe atom |
|---|---|---|---|---|
| | | Residue atom to | | to probe C22 |
| Residue | Residue atom | probe C22 (Å) | Probe atom | (Å) |
| D226 | O6840 (-δO—C22—)^{a} | 1.4 | — | — |
| L219 | H3390 (-δCH₃)^{b} | 4.5 | C21^{e} | 2.5 |
| K19 | H395 (-ζNH₃⁺)^{a} | 6.4 | C25—OH^{d} | 4.8 |
| H229 | N3539 (-δN═)^{a} | 7.0 | C15—OH^{d} | 5.2 |
| | H6836 (-εNH—)^{a} | 8.7 | C1—O—Ac^{d} | 8.7 |
| L217 | H3347 (-γCH₂—)^{b} | 8.3 | C12—O—(C═O)—CH₃^{e} | 5.2 |
| R278 | H4313 (-εNH—)^{a} | 9.8 | C11—O—(C═O)—CH₃^{d} | 8.8 |
| T223 | H3450 (-γOH)^{c} | 4.6 | C26═O^{d} | 4.4 |

Referring to FIG. 28A, five β-tubulin residues (R278, Q282, K19, H229, and K372) directly interact with 12 via H-bonds and salt bridges. FIG. 28B shows that T223 indirectly interacts with 12 via H₂O-bridged H-bonds. FIG. 28C shows the hydrophobic interactions between L217 and 12. FIG. 28D shows the hydrophobic interactions between L219 and 12.

Without wishing to be bound by theory, the analysis above suggests that the structures of the taccalonolide rings E and F and their direct interactions with β-tubulin residues (e.g., K19 and L219) are critical for the correct positioning of the taccalonolide core structure into its binding pocket to facilitate the covalent reaction between D226 and the 22,23-epoxide. It is also reasonable to hypothesize that other β-tubulin residues, including L217, H229, and R278, that interact with the taccalonolides at sites relatively remote from the site of the covalent interaction (FIG. 26A) are less important for covalent binding but may play an important role in mediating the post-reaction stability of the taccalonolide-tubulin complex. Thus, these findings that empirically establish the critical β-tubulin residues and the potential pharmacophore of taccalonolides may provide important guidance for the rational design and generation of “drug-like” taccalonolide analogues via strategies such as semi-synthesis and structural simplification (Wang, et al. (2019) Chem. Rev. 119, 4180-4220).

### 33. Flu-Tacca-7 as a Superior Tubulin Probe

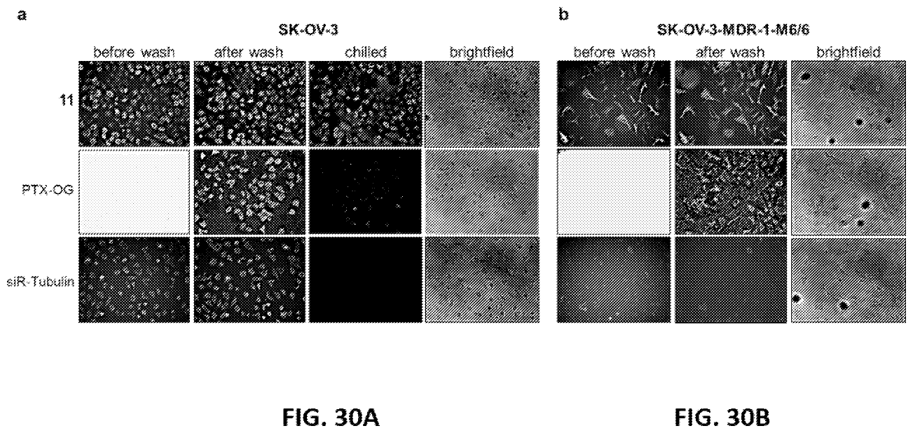

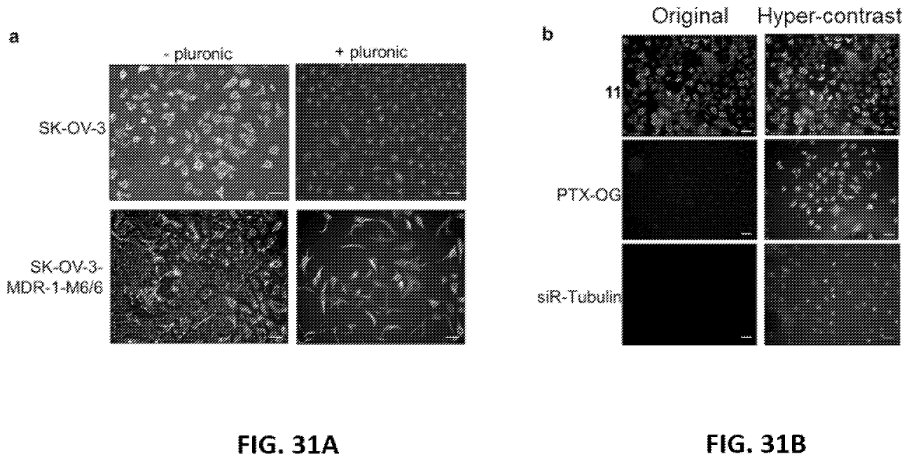

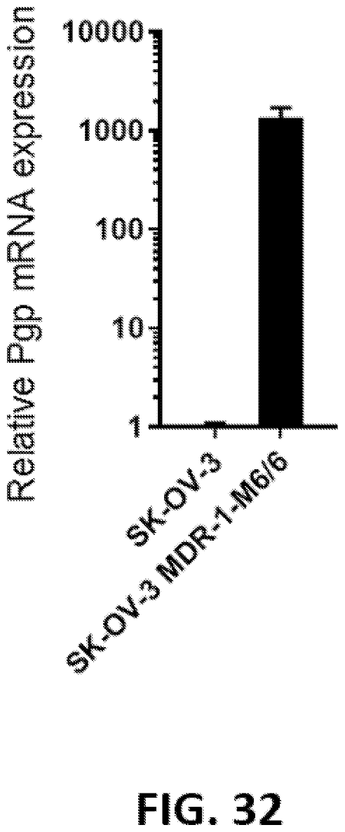

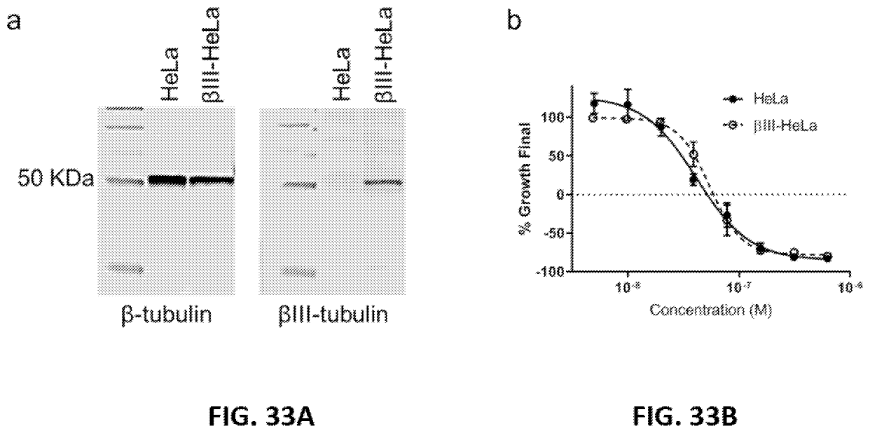

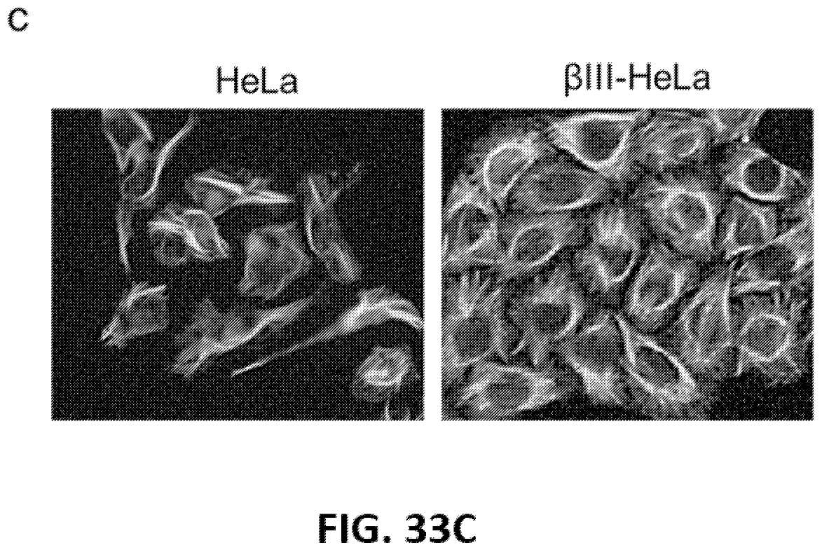

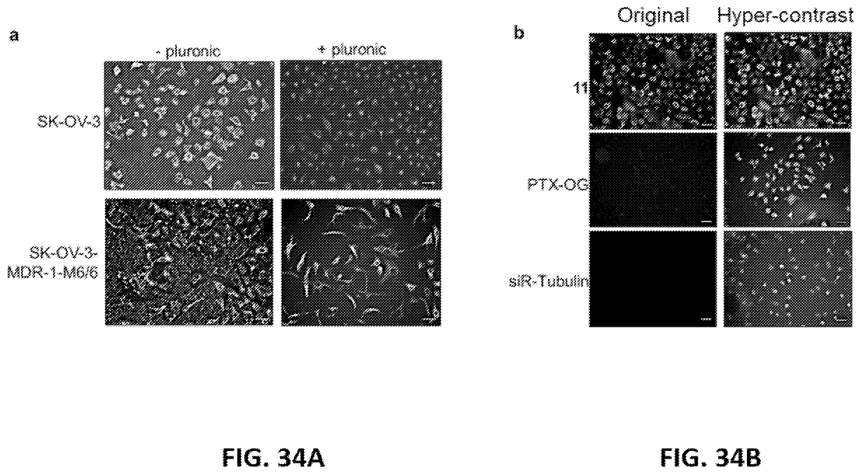

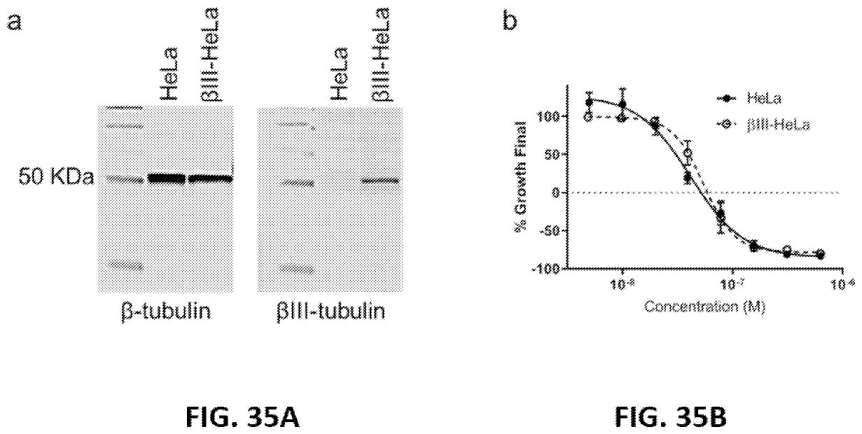

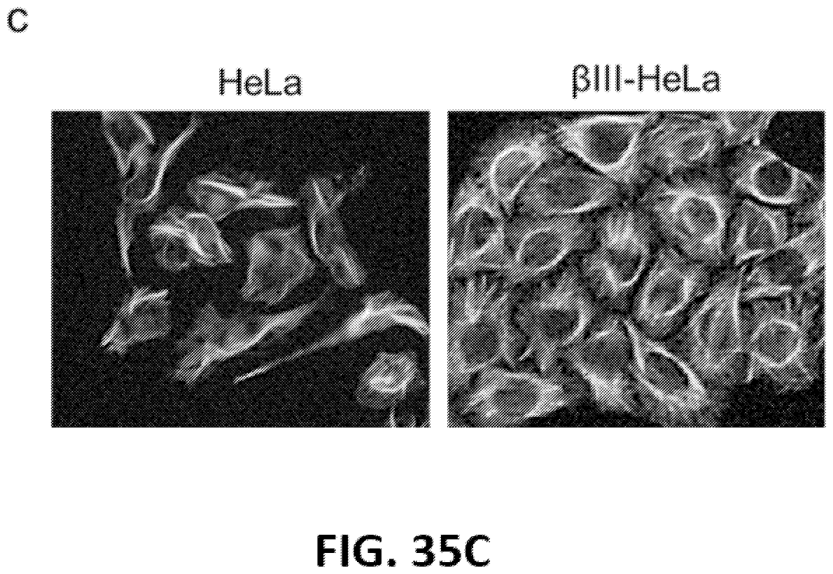

The Flu-tacca probes represent a class of irreversible, fluorogenic microtubule probes that can be utilized for cellular microtubule imaging and binding studies under conditions that have conventionally been unfavorable for the use of non-covalent probes. To demonstrate the utility of these cell-permeable probes for cellular imaging applications, the cellular microtubule staining activities of Flu-tacca-7 (11) were compared to Tubulin Tracker Green (Thermo Fisher Scientific, Oregon Green™ 488 Taxol, Bis-Acetate) and the far-red probe siR-Tubulin (Cytoskeleton), two commercial taxane-based probes commonly used for microtubule imaging in live cells. Tubulin Tracker Green was not amenable to visualization prior to washing excess probe from the media (FIG. 30A) and pluronic F-127 was necessary to facilitate drug loading into cells and decrease background fluorescence (FIG. 31A and FIG. 31B). In contrast, Flu-tacca-7 was effectively visualized without washing or the use of additional reagents to facilitate cell loading providing comparable convenience to the far-red probe siR-Tubulin (FIG. 30A). However, the intracellular staining of Flu-tacca-7 was superior to both Tubulin Tracker Green and siR-Tubulin when microtubules were depolymerized under chilled conditions or in cells expressing drug efflux transporters (FIG. 30A). Brief chilling was sufficient to destabilize microtubules in the presence of the commercial taxane probes, resulting in loss of probe signal, which could only be detected slightly over background when images were hyper-contrasted (FIG. 31A and FIG. 31B). In contrast, visualization of the taccalonolide probe 11 was retained at a similar level before and after chilling due to the covalent and irreversible nature of its binding to β-tubulin (FIG. 30A). The tubulin labeling efficacy of 11, Tubulin Tracker Green, and siR-Tubulin were also compared in SK-OV-3-MDR1-M6/6 ovarian cancer cells, which express 1000-fold increased levels of the P-glycoprotein drug efflux pump MDR-1 as compared to the parental SK-OV-3 cell line (FIG. 30B and FIG. 32). While 11 was effectively visualized in SK-OV-3-MDR-1-M6/6 cells, cellular staining of either Tubulin Tracker Green or siR-Tubulin was barely detectable above background in these multi-drug resistant cells (FIG. 30B). This observation is consistent with the fact that siR-Tubulin is recommended to be used in combination with verapamil to block probe efflux as both it and Tubulin Tracker Green are P-glycoprotein substrates. Additionally, 11 retained potency, efficacy, and microtubule binding in a βIII-tubulin expressing HeLa cell line (FIG. 33A-C), representing another clinically relevant form of taxane drug resistance. Therefore, the Flu-tacca irreversible microtubule probes disclosed herein are superior to commercial taxane-based probes, combining a high degree of specificity, brightness, and convenience with their ability to be used under conventionally unfavorable conditions (i.e., chilling and drug-resistant cells) without the need for additional pharmacological manipulations.

Referring to FIG. 30A, SK-OV-3 human ovarian cancer cells were treated with 0.5 μM of 11, Tubulin Tracker Green (PTX-OG; Thermo Fisher; middle), or siR-Tubulin (Cytoskeleton, Inc; bottom) for 5 h and imaged before treated media was removed (before wash), after the treated media were replaced by fresh medium (after wash), or after the treated cells were chilled at −20° C. for 20 min and fixed (chilled).

Referring to FIG. 30B, SK-OV-3-MDR1-M6/6 human ovarian cancer cells expressing P-glycoprotein were treated with 0.5 μM of each probe for 5 h and imaged at 37° C. before and after washing. The same image acquisition and visualization conditions were used for all images obtained for each probe. Brightfield images are shown in the far right column for each figure.

Referring to FIG. 31A, a comparison of Tubulin tracker green in SK-OV-3 or SK-OV-3-MDR-1-M6/6 cells with or without pluronic F-127 which facilitates probe loading and reduces background signal is shown.

Referring to FIG. 31B, images of chilled SK-OV-3 cells from FIG. 26A-D were hyper-contrasted to visualize low signal intensity. Scale bars=50 μm.

Referring to FIG. 32, levels of Pgp mRNA are over 1000× greater in the SK-OV-3 MDR-1-M6/6 cells than the parental SK-OV-3 cells. A one-tailed t-test was performed to give a P value of 0.0307 between the SK-OV-3 MDR-1-M6/6 cell line and the parental SK-OV-3 cells for Pgp expression where mean relative Pgp mRNA expression±SEM for n=2 biologically independent experiments each performed in duplicate.

Referring to FIG. 33A, the expression of total β-tubulin and the βIII-isotype of tubulin in HeLa cells and an isogenic line that overexpresses this isotype (βIII-HeLa) is shown.

Referring to FIG. 33B, the taccalonolide probe 11 retains antiproliferative and cytotoxic potency and efficacy in the βIII-tubulin expressing cell line (open circle) as compared to the parental HeLa cell line (closed circle). Each point represents mean±SEM from n=4 biologically independent experiments for βIII-HeLa cells and n=3 independent experiments for HeLa cells.

Referring to FIG. 33C, the taccalonolide probe 11 retains the ability to bind cellular microtubules in the βIII-tubulin expressing cell line as compared to the parental HeLa cell line. Cells were treated with 0.5 μM 11 for 5 h and imaged under identical acquisition conditions.

### 34. Evaluation of Taccalonolide Analogs

A summary of the activity of representative taccalonolide analogs is shown below.

TABLE 5

| | | GI₅₀ in |
|---|---|---|
| No. | Structure | HeLa (μM) |
| 158F | 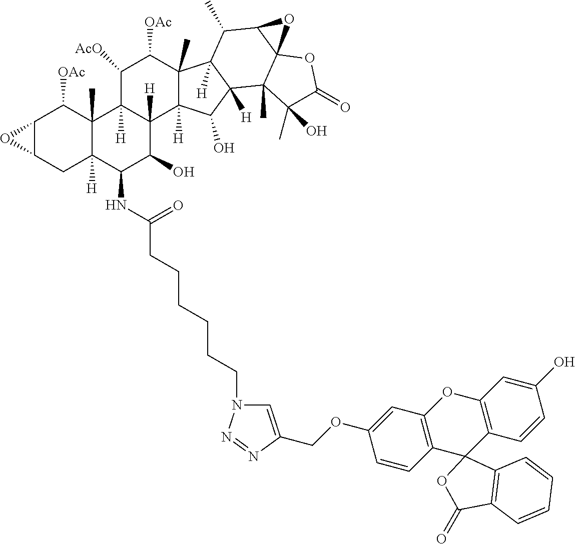 | 6 |
| 158E | 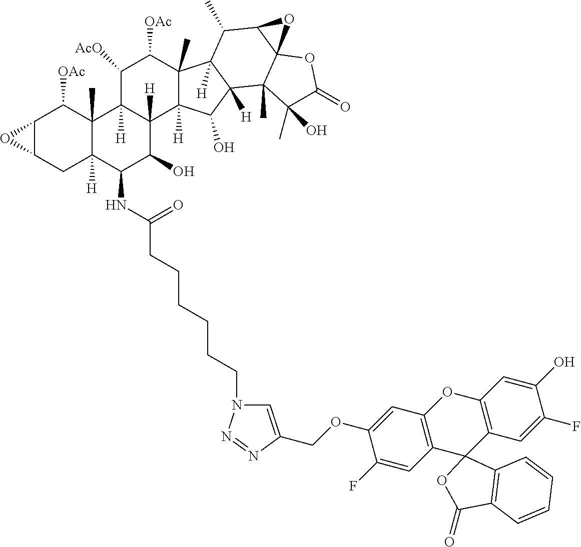 | >10 |
| 154J | 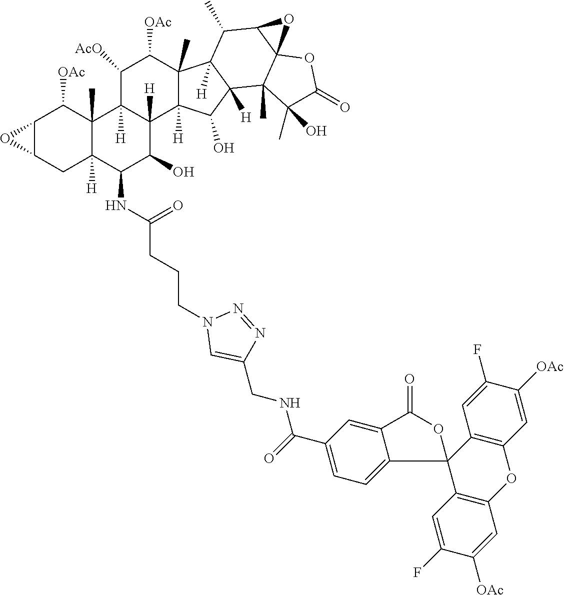 | >10 |
| 154B | 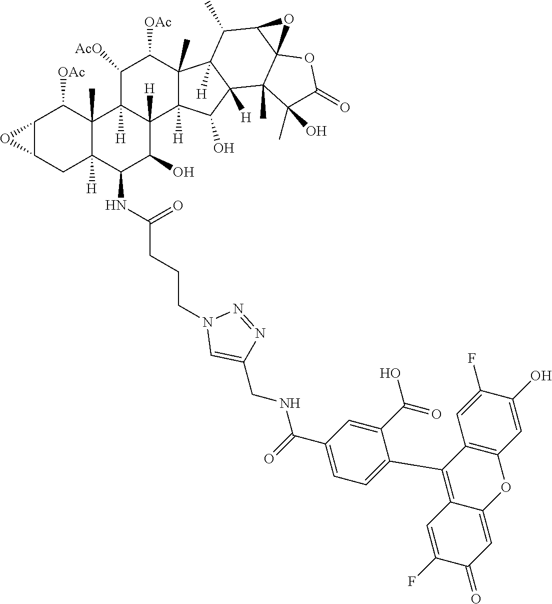 | >10 |
| 155G | 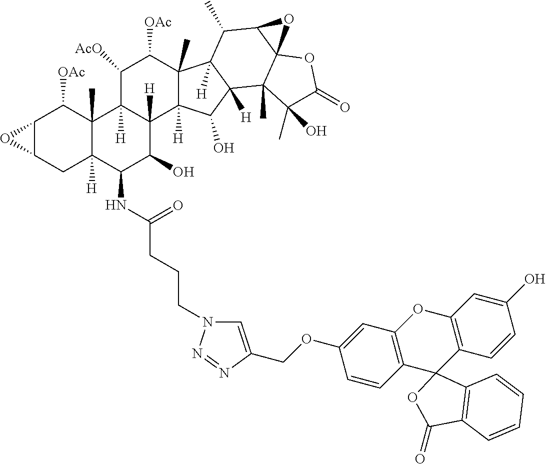 | 8.4 |
| 155F | 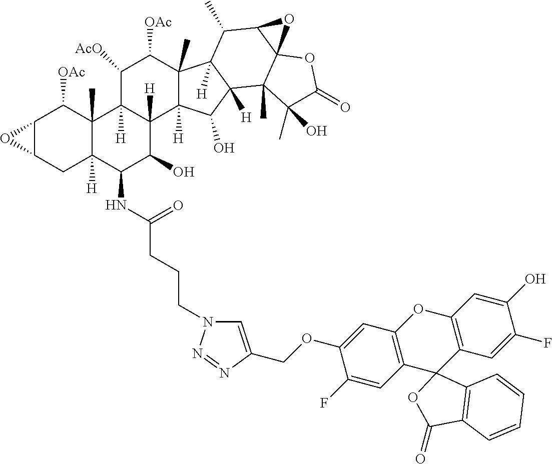 | 3.7 |
| 152D | 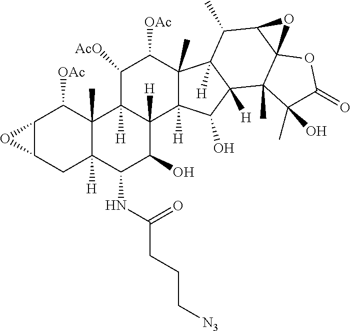 | 0.12 |
| 152E | 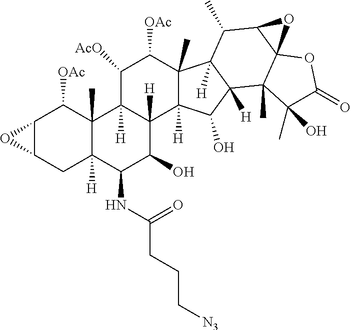 | 0.09 |
| 154C | 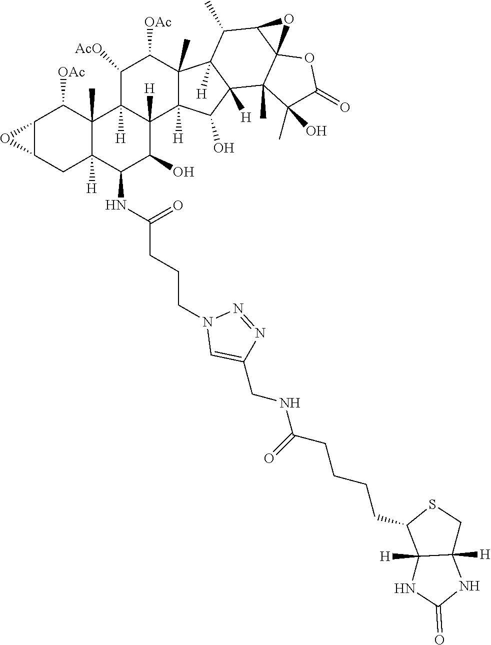 | >10 |
| 162C | 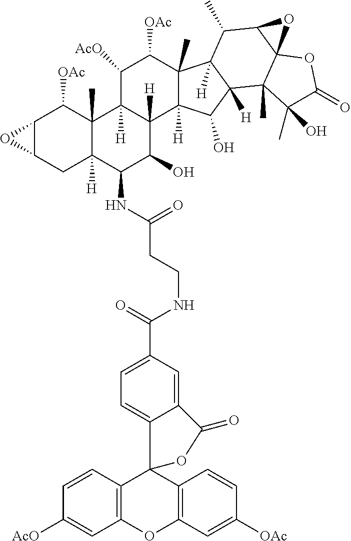 | 10 |
| 163E | 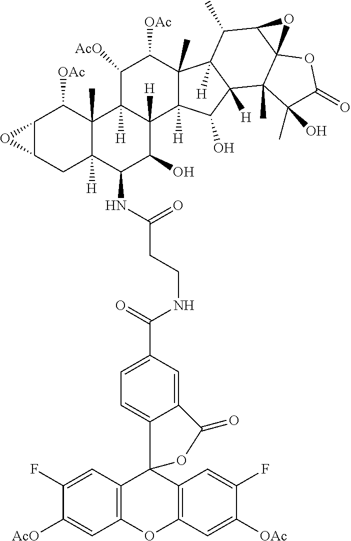 | >10 |
| 163D | 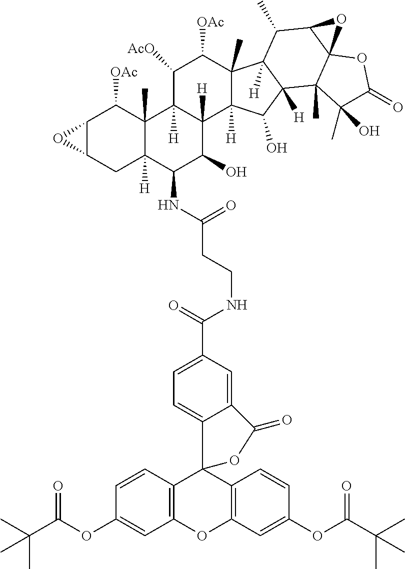 | 2 |
| 164E | 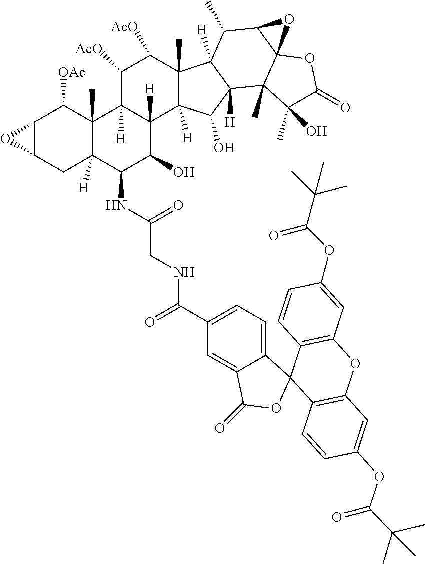 | 0.74 |
| 164B | 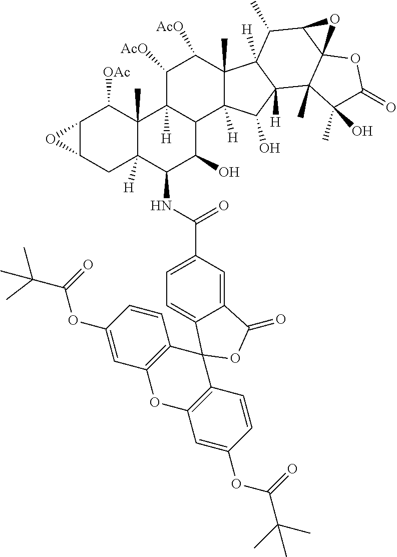 | 0.031 |
| 167C | 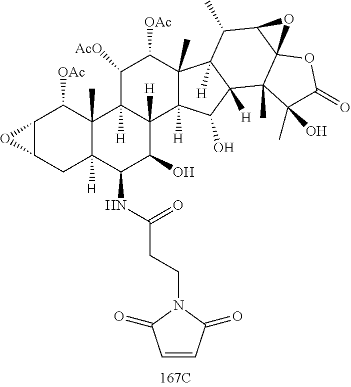 | — |
| 171B | 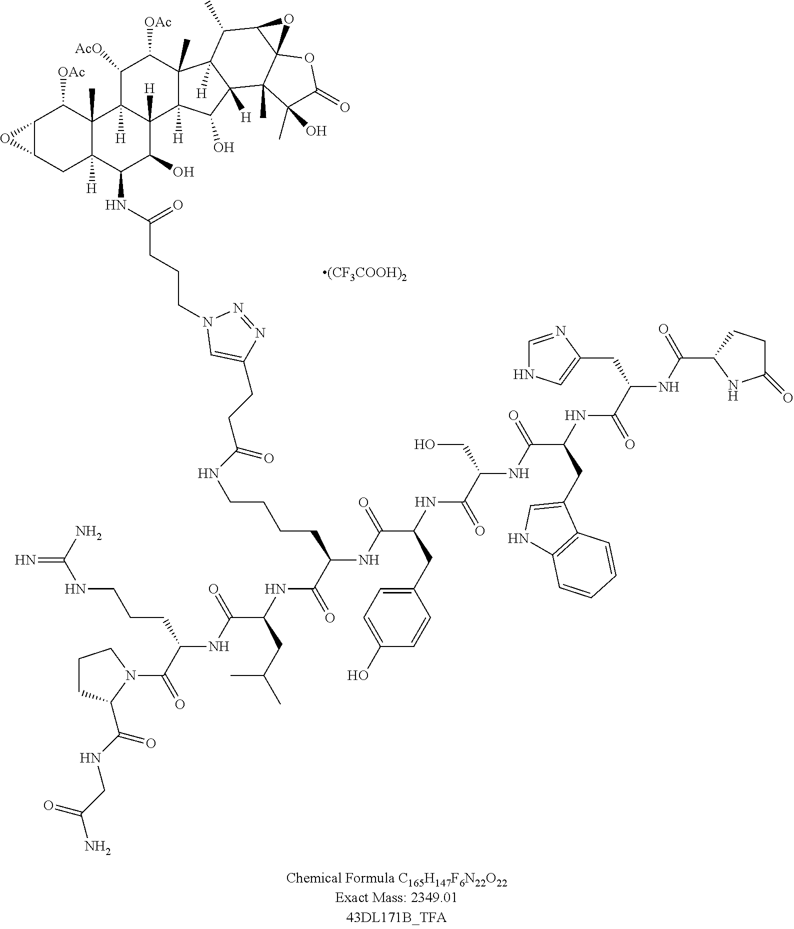 | 2.6 |

In sum, stable tacca-conjugates that retain the biological properties of taccalonolides have been prepared. Without wishing to be bound by theory, this linking technology can generate stable tacca-ADC conjugates, which may lead to new tumor-targeted therapeutic methods, especially for taxol-resistant cancers. In addition, the disclosed protocols can be useful in generating new tubulin labeling probes (e.g., fluorescent tags and biotin) that exhibit distinct and superior biological properties compared to the commercial taxol-based probes. As the taxol-based probes bind tubulin non-covalently (i.e., unstable) they often provide inconsistent labeling results, especially for quantificational studies. In contrast, the disclosed tacca-based probes generate permanent (i.e., covalent binding) labeling of tubulin and won't be compromised during sample processing/manipulation and retain efficacy under conditions that limit the use of commercial taxane probes, including cold and in the presence of drug efflux pumps. Thus, the disclosed analogs may be particularly good for quantificational studies.

### 35. Elucidating Target Specificity of the Taccalonolide Covalent Microtubule Stabilizers Employing a Combinatorial Chemical Approach

While three distinct classes of covalent microtubule stabilizing agents (i.e. the taccalonolides, cyclostreptin and dactylolide/zampanolide) have been evaluated for their potential as cancer therapeutics that circumvent taxane associated drug resistance in biochemical and cellular assays³², the taccalonolides are the only class that has demonstrated in vivo efficacy in both drug sensitive and resistant tumor models^{17,20,21,25}. Here we describe the development of a combinatorial chemical proteomics approach that enabled confirmation of the target specificity of taccalonolide binding to β-tubulin in cellular assays and identification of key structural features of both the taccalonolides and β-tubulin that are critical for drug-target binding. Our combinatorial strategy to empirically establish the key taccalonolide binding residues in cell-based assays should be broadly useful for detailed ligand-protein binding studies and structure-based optimization of quite a number of existing drugs and/or drug candidates that covalently modify their targets.

While the immunofluorescence and immunoblotting with the Flu-tacca probes clearly demonstrate that the predominant cellular target of the taccalonolides is indeed a covalent interaction with β-tubulin, we cannot rule out the possibility of other minor covalent or non-covalent targets. One important consideration is the finding that taccalonolides lacking the critical covalent binding moiety, the 22,23-epoxide, show no antiproliferative, cytotoxic, or microtubule bundling activities in cells and do not directly interact with tubulin in biochemical assays. The lack of any detectable target engagement or bioactivity of probes lacking this epoxide in the current study lends further support to this point.

An unanticipated finding from the generation of the Flu-tacca probes in this study is the identification of a strategy to improve the binding affinity and microtubule stabilizing potency of the taccalonolides by targeting a binding pocket nearby the taccalonolide binding site that engages the M-loop of β-tubulin, which is integral in pharmacological microtubule stabilization^{18,35}. These findings provide an opportunity to develop additional ‘drug-like’ taccalonolide analogues with sub-nanomolar cellular potency, which is desired for targeted drug delivery strategies (i.e. peptide- and antibody-drug conjugates) and paves the way for further development of the taccalonolides as microtubule stabilizers for cancer therapy. A further evaluation of the critical taccalonolide moieties and β-tubulin residues that mediate binding and biological activity will continue to provide mechanistic insight to optimize our models of taccalonolide binding and design more optimal semi-synthetic and possibly fully-synthetic taccalonolide-like compounds that could have efficacy in drug resistant models due to their irreversible target binding. Additionally, the fluorogenic taccalonolide-based probes represent highly specific, irreversible tubulin-labeling probes that have superior utilities as compared to commercial taxane-based probes.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 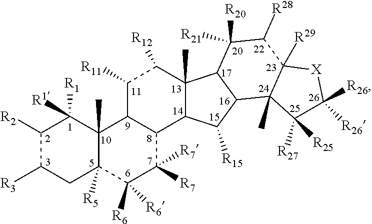
wherein each occurrence of - - - - - - is a single or double covalent bond;
wherein X is selected from O, NR^{x}, and C(R^{x})₂;
wherein each occurrence of R^{x}, when present, is independently selected from hydrogen and C1-C6 alkyl;
wherein R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy1, Ar1, (C1-C12 alkyl)Ar1, and —OAr¹;
wherein each occurrence of R³¹, R³², R³⁴, R^{35a}, and R^{35b}, when present, is independently selected from hydrogen and C1-C12 alkyl;
wherein each occurrence of R³³, when present, is independently selected from hydrogen, C1-C12 alkyl, and monocyclic aryl monosubstituted with a methyl group;
wherein each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino;
wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and
wherein R^{1′} is hydrogen;
or wherein each of R¹ and R^{1′} together comprise ═O or ═NR³⁶;
wherein each occurrence of R³⁶, when present, is independently selected from hydrogen and C1-C12 alkyl;
wherein each of R² and R³ is independently selected from hydrogen, —OH, C1-C12 hydroxy, and halogen;
or wherein each of R₂ and R₃ together comprise —O—;
wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino;
or wherein R⁵ is absent;
wherein each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr¹, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)R⁴⁰, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰;
wherein each occurrence of R⁴⁰, when present, is independently a C1-C30 alkyl functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide;
wherein each occurrence of R⁴¹ and R⁴², when present, is independently selected from hydrogen and C1-C12 alkyl;
wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴;
wherein each occurrence of Ar⁴, when present, is a structure represented by a formula selected from: 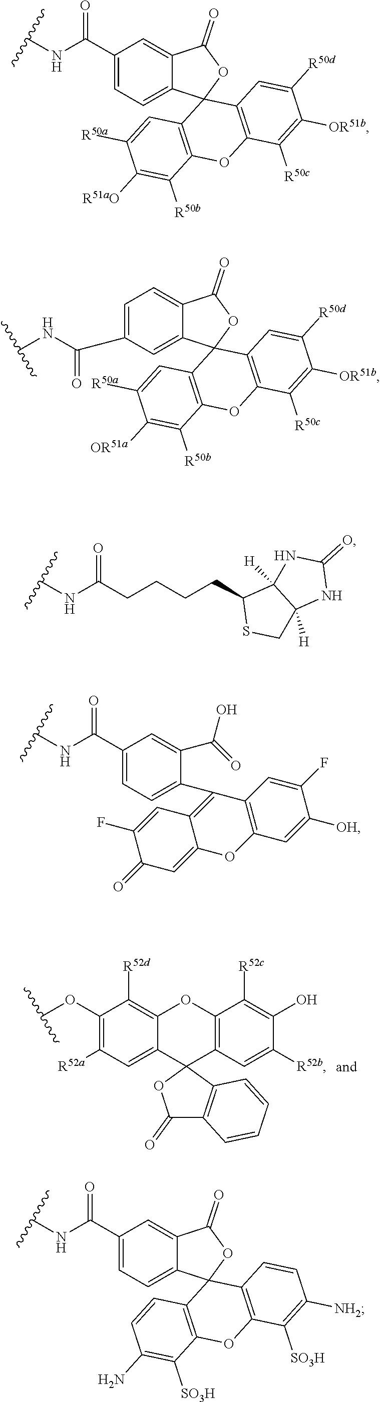
wherein each of R^{50a}, R^{50b}, R^{50c}, and R^{50d}, when present, is independently selected from hydrogen, —F, and —Cl;
wherein each of R^{51a} and R^{51b}, when present, is independently selected from hydrogen and —C(O)(C1-C12 alkyl);
wherein each of R^{52a}, R^{52b}, R^{52c}, and R^{52d}, when present, is independently selected from hydrogen, —F, and —Cl;
wherein each occurrence of Ar³, when present, is a structure represented by a formula selected from: 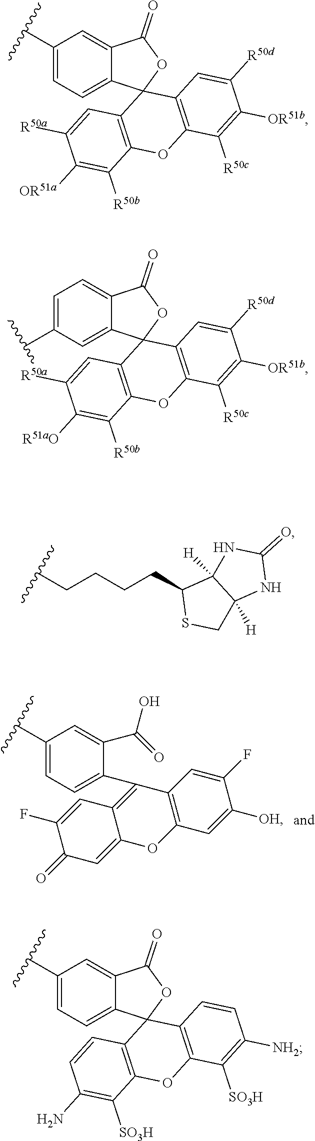
or wherein one of R⁶ and R^{6′} is absent;
wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl);
or wherein each of R⁷ and R^{7′} together comprise ═O;
or wherein one of R⁷ and R^{7′} is absent;
wherein each of R¹¹ and R¹² is independently selected from hydrogen, —OH, C1-C8 hydroxy, C1-C6 alkyl, C1-C8 alkoxy, and —OC(O)(C1-C8 alkyl);
wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide);
wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl);
wherein R²¹ is selected from hydrogen and C1-C6 alkyl;
wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide);
wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino;
wherein each of R²⁶ and R^{26′} is independently selected from hydrogen, —OH, C1-C8 hydroxy, and C1-C8 alkoxy;
or wherein each of R²⁶ and R^{26′} together comprise ═O;
wherein R²⁷ is selected from hydrogen and C1-C6 alkyl;
wherein each of R²⁸ and R²⁹ is independently selected from hydrogen and halogen;
or wherein each of R²⁸ and R²⁹ together comprise —O— or —N(R³⁷)—;
wherein R³⁷, when present, is selected from hydrogen, C1-C4 alkyl, —SO₂R⁷¹, and a structure having a formula: 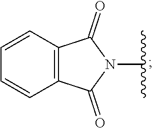 [SMILES: *N1C(=O)c2ccccc2C1=O]
and
wherein R⁷¹, when present, is selected from hydrogen, C1-C4 alkyl, —CH₂CH₂Si(CH₃)₃, and monocyclic aryl monosubstituted with a methyl group,
provided that at least one of R⁶ and R^{6′} is —NR⁴¹C(O)(C1-C30 alkyl), —NR⁴¹C(O)Ar², —NR⁴¹C(O)(C1-C30 alkyl)Ar2, —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, or —NR⁴¹C(O)R⁴⁰, and
provided that when one or both of R²⁸ and R²⁹ is hydrogen then the occurrence of - - - - - - at C-22/C-23 is a double covalent bond,
or a pharmaceutically acceptable salt thereof.

2. The compound of claim 1, wherein R⁶ is selected from —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴¹ and R^{6′} is hydrogen.

3. The compound of claim 1, wherein R⁶ is —NR⁴¹C(O)R⁴⁰.

4. The compound of claim 1, wherein R⁴⁰ is a C1-C30 alkyl functionalized with a maleimide group.

5. The compound of claim 1, wherein R⁴⁰ is a structure:

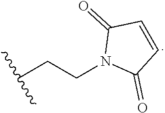 [SMILES: *CCN1C(=O)C=CC1=O]

6. The compound of claim 1, each occurrence of Ar2, when present, is triazolyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar³, and Ar³.

7. The compound of claim 1, wherein each occurrence of Ar², when present, is triazolyl substituted with 1 Ar³ group.

8. The compound of claim 1, having a structure represented by a formula:

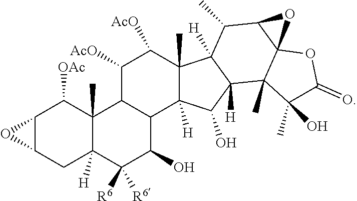

9. The compound of claim 1, having a structure represented by a formula:

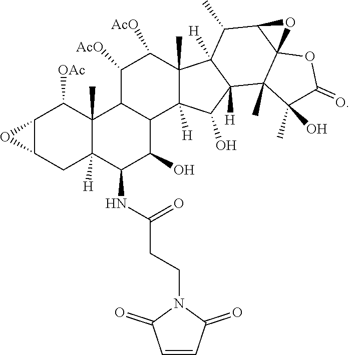 [SMILES: CC(=O)O[C@H]1C2C([C@@H](O)[C@@H](NC(=O)CCN3C(=O)C=CC3=O)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

10. The compound of claim 1, having a structure represented by a formula:

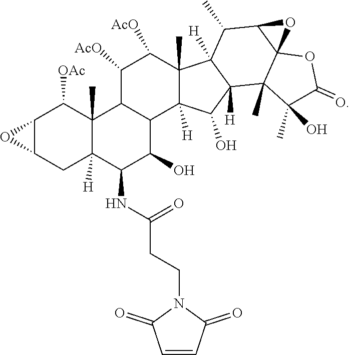 [SMILES: CC(=O)O[C@H]1C2C([C@@H](O)[C@@H](NC(=O)CCN3C(=O)C=CC3=O)[C@H]3C[C@@H]4O[C@@H]4[C@H](OC(C)=O)[C@]23C)[C@@H]2[C@@H](O)[C@@H]3[C@H]([C@H](C)[C@H]4O[C@]45OC(=O)[C@@](C)(O)[C@]35C)[C@@]2(C)[C@H]1OC(C)=O]

11. The compound of claim 1, having a structure selected from:

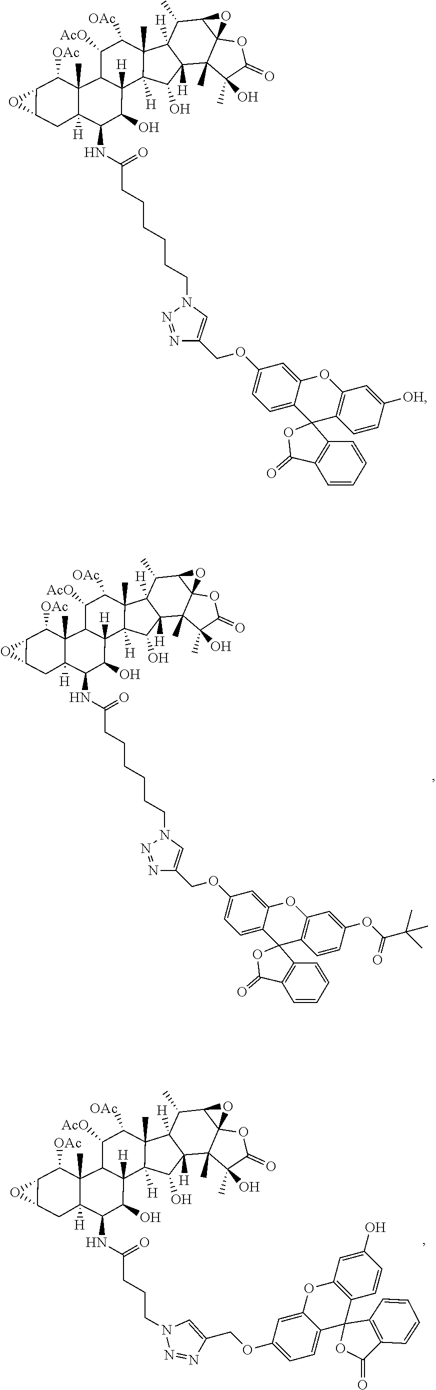

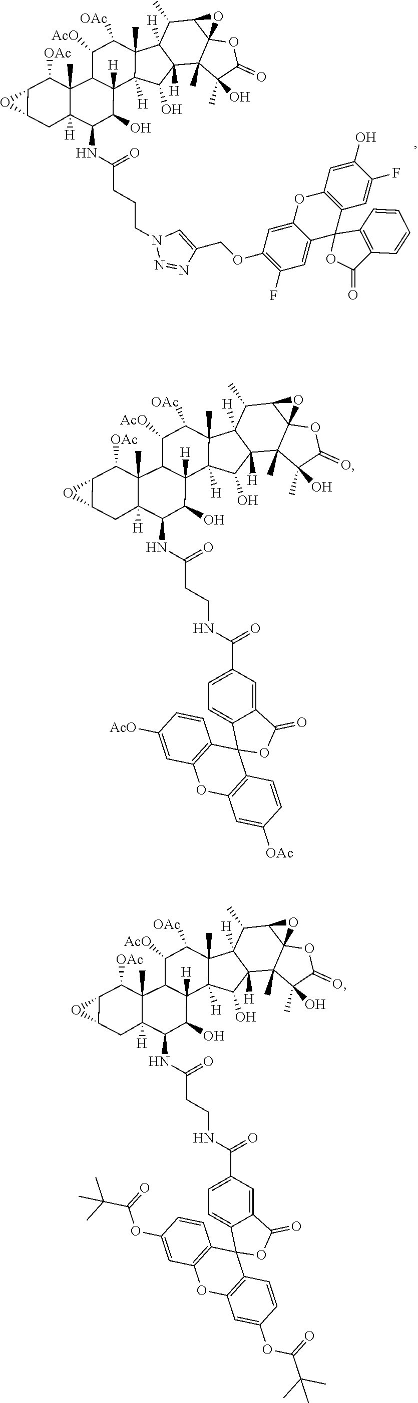

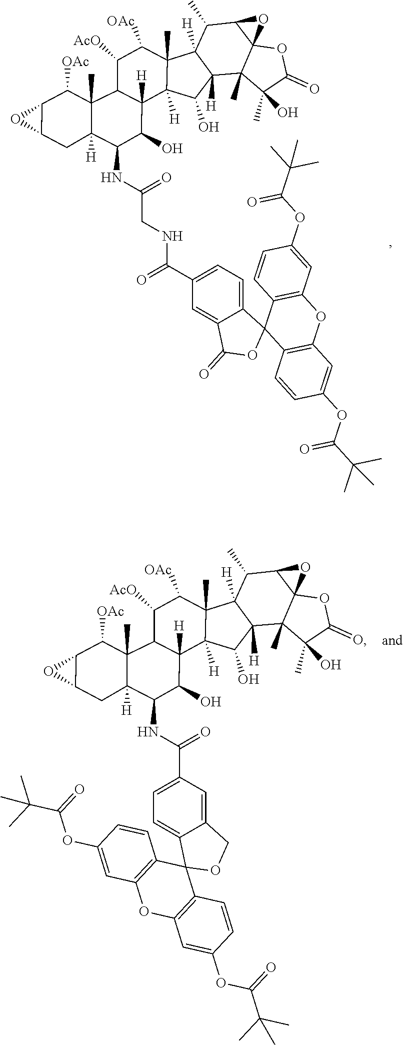

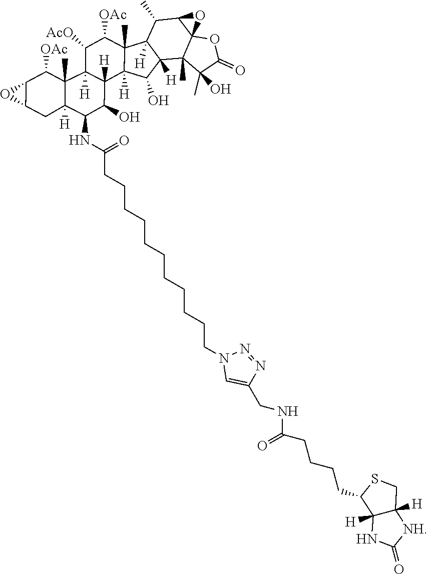

12. The compound of claim 1, having a structure selected from:

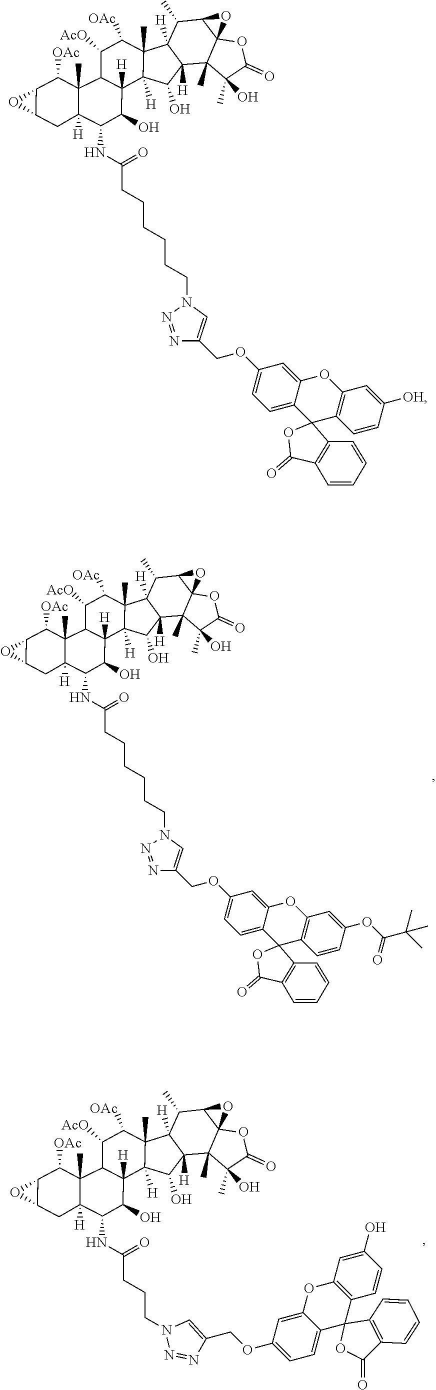

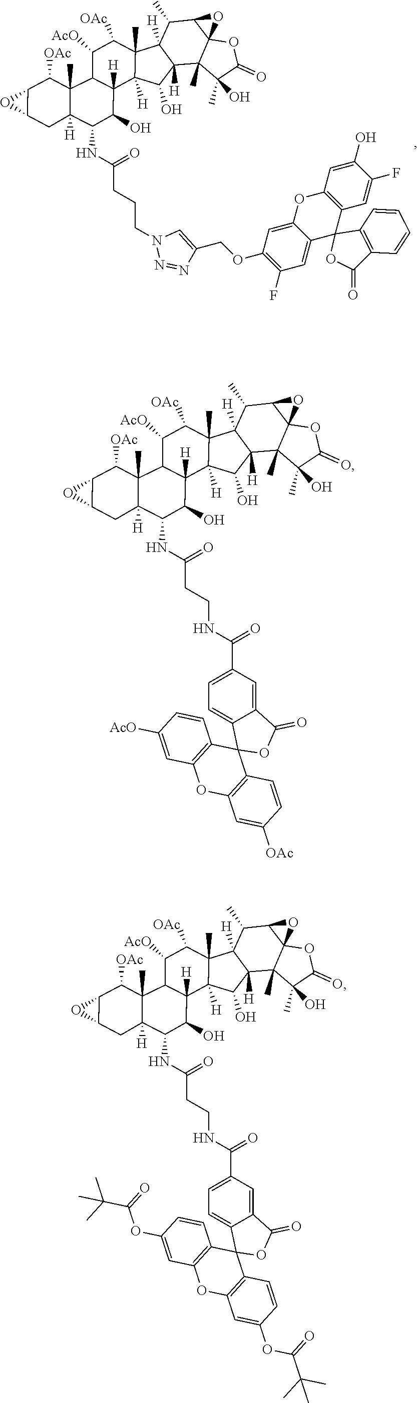

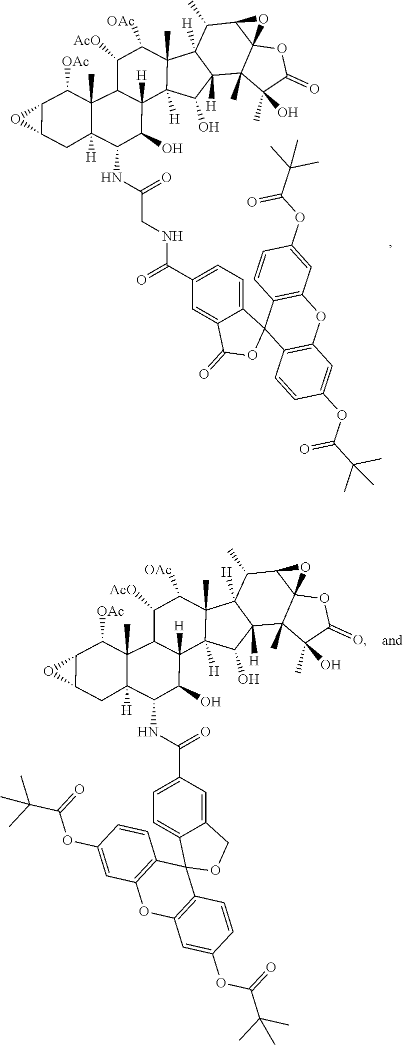

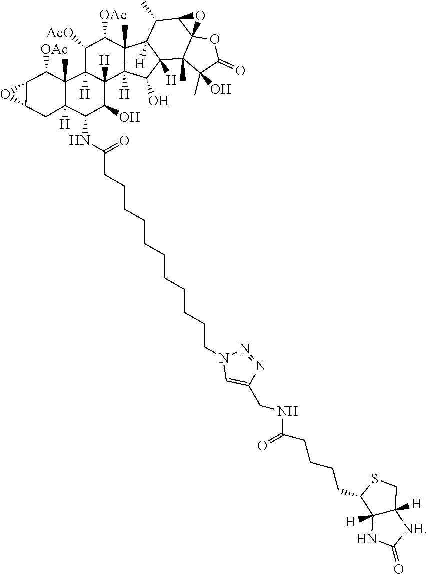

13. A pharmaceutical composition comprising a therapeutically effective amount of the compound of claim 1, and a pharmaceutically acceptable carrier.

14. The composition of claim 13, wherein the composition comprises at least 90 wt % of the compound, based on the total weight of the composition.

15. A method for the treatment of a hyperproliferative disorder in a subject, the method comprising administering to the subject an effective amount of at least one compound of claim 1.

16. The method of claim 15, wherein the subject is a mammal.

17. The method of claim 15, wherein the subject has been diagnosed with a need for treatment of a hyperproliferative disorder prior to the administering step.

18. The method of claim 15, further comprising the step of identifying a subject in need of treatment of a hyperproliferative disorder.

19. The method of claim 15, wherein the hyperproliferative disorder is a cancer.

20. A kit comprising at least one compound of claim 1, and one or more of:
(a) at least one agent associated with the treatment of a hyperproliferative disorder;
(b) instructions for administering the compound in connection with treating a hyperproliferative disorder; and
(c) instructions for treating a hyperproliferative disorder.

21. A compound having a structure represented by a formula: 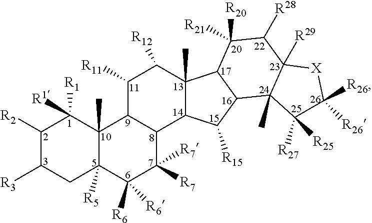
wherein each occurrence of - - - - - - is a single or double covalent bond;
wherein X is selected from O, NR^{x}, and C(R^{x})₂;
wherein each occurrence of R^{x}, when present, is independently selected from hydrogen and C1-C6 alkyl;
wherein R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹;
wherein each occurrence of R³¹, R³², R³⁴, R^{35a}, and R^{35b}, when present, is independently selected from hydrogen and C1-C12 alkyl;
wherein each occurrence of R³³, when present, is independently selected from hydrogen, C1-C12 alkyl, and monocyclic aryl monosubstituted with a methyl group;
wherein each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino;
wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and
wherein R^{1′} is hydrogen;
or wherein each of R¹ and R^{1′} together comprise ═O or ═NR₃₆;
wherein each occurrence of R³⁶, when present, is independently selected from hydrogen and C1-C12 alkyl;
wherein each of R² and R³ is independently selected from hydrogen, —OH, C1-C12 hydroxy, and halogen;
or wherein each of R₂ and R₃ together comprise —O—;
wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino;
or wherein R⁵ is absent;
wherein each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr1, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)R⁴⁰, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰;
wherein each occurrence of R⁴⁰, when present, is independently a C1-C30 alkyl functionalized with a group selected from —SH, —NH2, —NHOH, terminal alkyne, haloacetyl, maleimide, isothiocyanate, N-hydroxysuccinimde, succinimidyl ester, tetrafluorophenyl ester, sulfodichlorophenol ester, and hydrazide;
wherein each occurrence of R⁴¹ and R⁴², when present, is independently selected from hydrogen and C1-C12 alkyl;
wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴;
wherein each occurrence of Ar⁴, when present, is a structure represented by a formula: 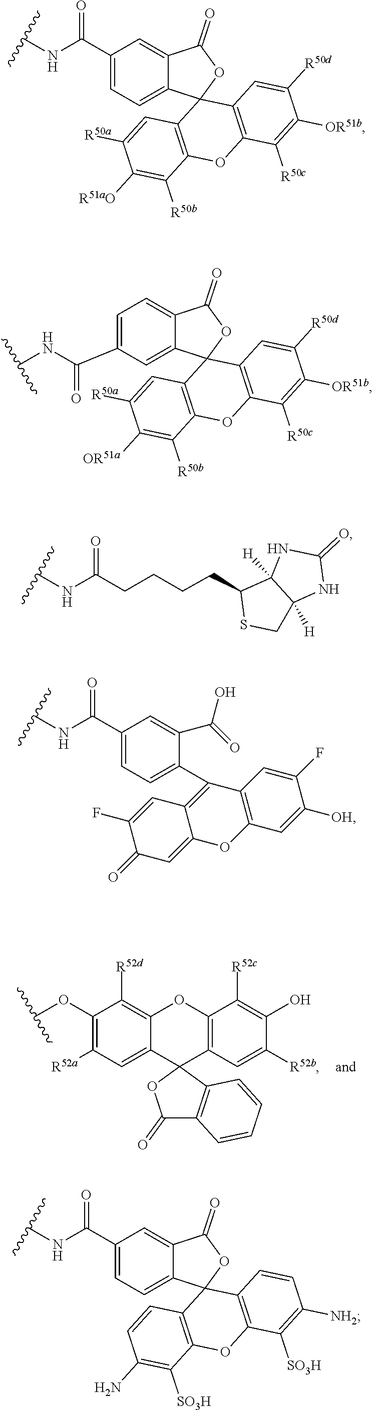
wherein each of R^{50a}, R^{50b}, R^{50c}, and R^{50d}, when present, is independently selected from hydrogen, —F, and —Cl;
wherein each of R^{51a} and R^{51b}, when present, is independently selected from hydrogen and —C(O)(C1-C12 alkyl);
wherein each of R^{52a}, R^{52b}, R^{52c}, and R^{52d}, when present, is independently selected from hydrogen, —F, and —Cl;
wherein each occurrence of Ar³, when present, is a structure represented by a formula selected from: 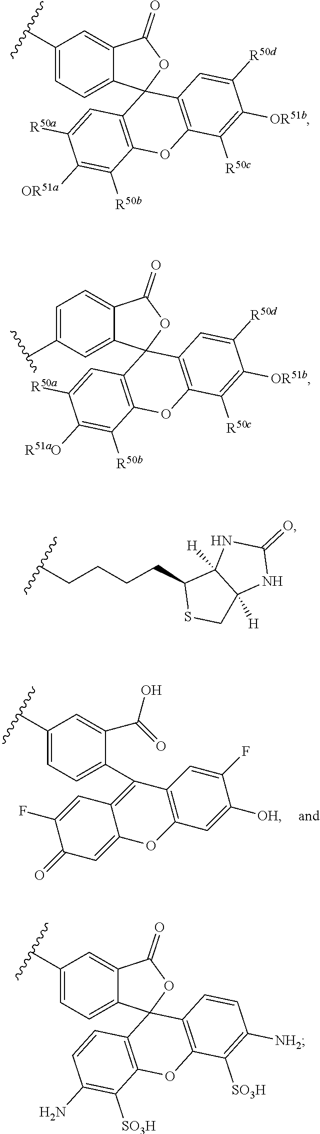
or wherein one of R⁶ and R^{6′} is absent;
wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl);
or wherein each of R⁷ and R^{7′} together comprise ═O;
or wherein one of R⁷ and R^{7′} is absent;
wherein each of R¹¹ and R¹² is independently selected from hydrogen, —OH, C1-C8 hydroxy, C1-C6 alkyl, C1-C8 alkoxy, and —OC(O)(C1-C8 alkyl);
wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide);
wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl);
wherein R²¹ is selected from hydrogen and C1-C6 alkyl;
wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide);
wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino;
wherein each of R²⁶ and R^{26′} is independently selected from hydrogen, —OH, C1-C8 hydroxy, and C1-C8 alkoxy;
or wherein each of R²⁶ and R^{26′} together comprise ═O;
wherein R²⁷ is selected from hydrogen and C1-C6 alkyl;
wherein each of R²⁸ and R²⁹ is independently selected from hydrogen and halogen;
or wherein each of R²⁸ and R²⁹ together comprise —O— or N(R³⁷)—;
wherein R³⁷, when present, is selected from hydrogen, C1-C4 alkyl, —SO₂R⁷¹, and a structure having a formula: 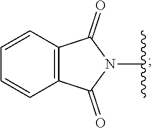 [SMILES: *N1C(=O)c2ccccc2C1=O]
and
wherein R⁷¹, when present, is selected from hydrogen, C1-C4 alkyl, —CH₂CH₂Si(CH₃)₃, and monocyclic aryl monosubstituted with a methyl group,
provided that at least one of R⁶ and R^{6′} is —OC(O)R⁴⁰ or —NR⁴¹C(O)R⁴⁰, and
provided that when one or both of R²⁸ and R²⁹ is hydrogen then the occurrence of - - - - - at C-22/C-23 is a double covalent bond,
or a pharmaceutically acceptable salt thereof.

22. The compound of claim 21, wherein R⁴⁰ is a C1-C30 alkyl functionalized with a maleimide group.

23. The compound of claim 21, wherein R⁴⁰ is a structure:

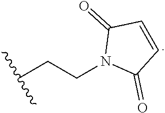 [SMILES: *CCN1C(=O)C=CC1=O]

24. A compound having a structure represented by a formula: 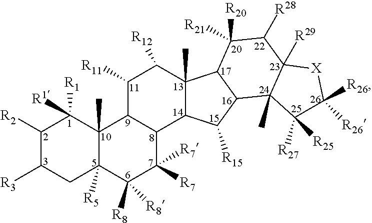
wherein each occurrence of - - - - - - is a single or double covalent bond;
wherein X is selected from O, NR^{x}, and C(R^{x})₂;
wherein each occurrence of R^{x}, when present, is independently selected from hydrogen and C1-C6 alkyl;
wherein R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹;
wherein each occurrence of R³¹, R³², R³⁴, R^{35a}, and R^{35b}, when present, is independently selected from hydrogen and C1-C12 alkyl;
wherein each occurrence of R³³, when present, is independently selected from hydrogen, C1-C12 alkyl, and monocyclic aryl monosubstituted with a methyl group;
wherein each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino;
wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and
wherein R^{1′} is hydrogen;
or wherein each of R¹ and R^{1′} together comprise ═O or ═NR³⁶;
wherein each occurrence of R³⁶, when present, is independently selected from hydrogen and C1-C12 alkyl;
wherein each of R2 and R3 is independently selected from hydrogen, —OH, C1-C12 hydroxy, and halogen;
or wherein each of R₂ and R₃ together comprise —O—;
wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino;
or wherein R⁵ is absent;
wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl);
or wherein each of R⁷ and R^{7′} together comprise ═O;
or wherein one of R⁷ and R^{7′} is absent;
wherein each of R⁸ and R^{8′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr¹, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, —NR⁴¹C(O)(C1-C30 alkyl)-L-Z, —NR⁴¹C(O)(C1-C30 alkyl)-L-(C1-C30 alkyl)-Z, and —NR⁴¹C(O)-L-(C1-C30 alkyl)-Z;
wherein L is a linker;
wherein Z is selected from an antibody, an antibody fragment, a vitamin, a hormone, a carbohydrate, a molecular ligand, an aptamer, a non-antibody protein, a peptide, a nucleic acid, a fluorophore, and a drug;
wherein each occurrence of R⁴¹ and R⁴², when present, is independently selected from hydrogen and C1-C12 alkyl;
wherein each occurrence of Ar², when present, is independently selected from aryl and heteroaryl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴;
wherein each occurrence of Ar⁴, when present, is a structure represented by a formula: 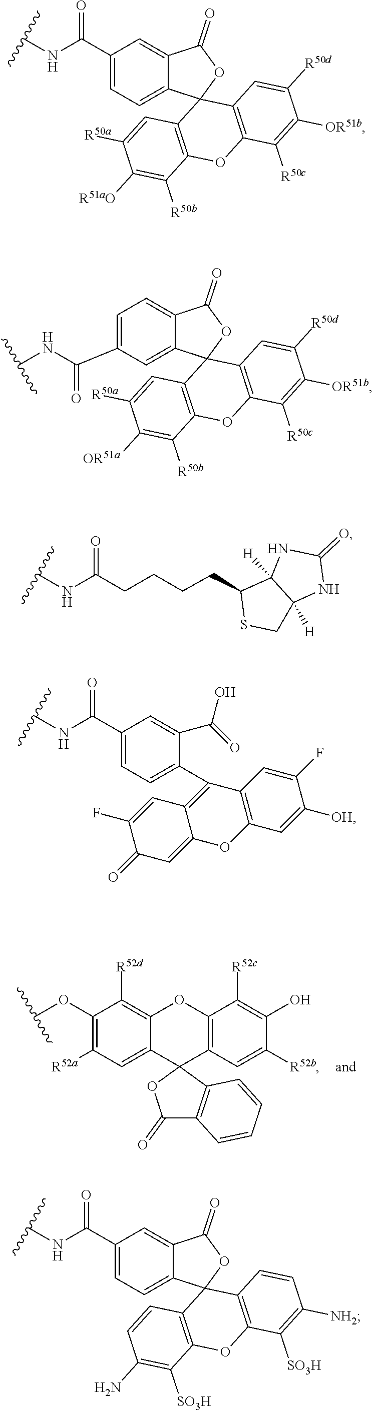
wherein each of R^{50a}, R^{50b}, R^{50c}, and R^{50d}, when present, is independently selected from hydrogen, —F, and —Cl;
wherein each of R^{51a} and R^{51b}, when present, is independently selected from hydrogen and —C(O)(C1-C12 alkyl);
wherein each of R^{52a}, R^{52b}, R^{52c}, and R^{52d}, when present, is independently selected from hydrogen, —F, and —Cl;
wherein each occurrence of Ar³, when present, is a structure represented by a formula selected from: 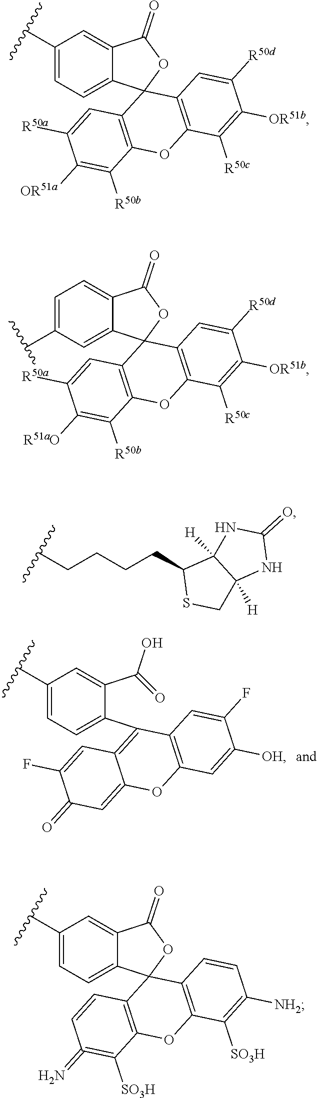
or wherein one of R⁸ and R^{8′} is absent;
wherein each of R¹¹ and R¹² is independently selected from hydrogen, —OH, C1-C8 hydroxy, C1-C6 alkyl, C1-C8 alkoxy, and —OC(O)(C1-C8 alkyl);
wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide);
wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl);
wherein R²¹ is selected from hydrogen and C1-C6 alkyl;
wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide);
wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino;
wherein each of R²⁶ and R^{26′} is independently selected from hydrogen, —OH, C1-C8 hydroxy, and C1-C8 alkoxy;
or wherein each of R²⁶ and R^{26′} together comprise ═O;
wherein R²⁷ is selected from hydrogen and C1-C6 alkyl;
wherein each of R²⁸ and R²⁹ is independently selected from hydrogen and halogen;
or wherein each of R²⁸ and R²⁹ together comprise —O— or N(R³⁷)—;
wherein R³⁷, when present, is selected from hydrogen, C1-C4 alkyl, —SO₂R⁷¹, and a structure having a formula: 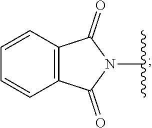 [SMILES: *N1C(=O)c2ccccc2C1=O]
and
wherein R⁷¹, when present, is selected from hydrogen, C1-C4 alkyl, —CH₂CH₂Si(CH₃)₃, and monocyclic aryl monosubstituted with a methyl group,
provided that one and only one of R⁸ and R^{8′} is —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl)-L-Z, —NR⁴¹C(O)(C1-C30 alkyl)-L-(C1-C30 alkyl)-Z, or —NR⁴¹C(O)-L-(C1-C30 alkyl)-Z, and
provided that when one or both of R²⁸ and R²⁹ is hydrogen then the occurrence of - - - - - at C-22/C-23 is a double covalent bond,
or a pharmaceutically acceptable salt thereof.

25. The compound of claim 24, wherein L is selected from —NR⁶¹C(O)—, —C(O)NR⁶¹—, —NR⁶¹C(S)NR⁶²—, —SCH₂C(O)—, —C(O)SCH₂—, 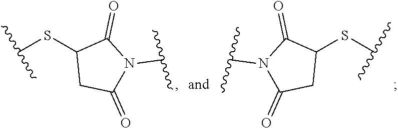 [SMILES: *SC1CC(=O)N(*)C1=O ; *SC1CC(=O)N(*)C1=O]
and
wherein each of R⁶¹ and R⁶², when present, is independently selected from hydrogen and C1-C12 alkyl.

26. The compound of claim 24, wherein L is selected from

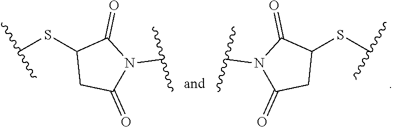 [SMILES: *SC1CC(=O)N(*)C1=O ; *SC1CC(=O)N(*)C1=O]

27. The compound of claim 24, wherein Z is selected from an antibody and an antibody fragment.

28. The compound of claim 24, wherein the compound is:

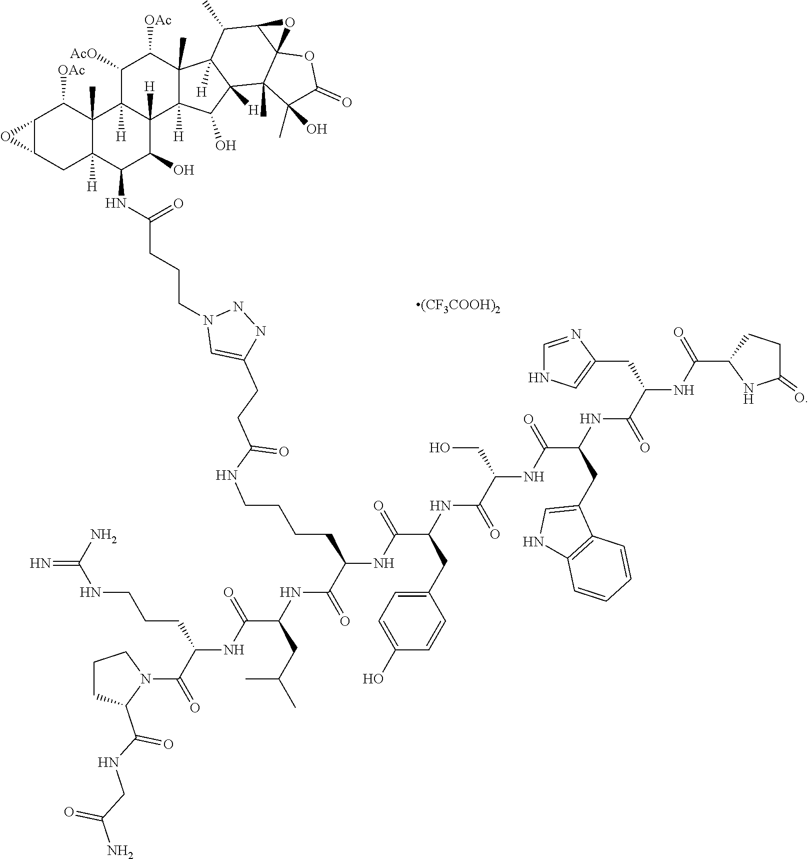

29. A method of making a conjugated taccalonolide compound having a structure represented by a formula: 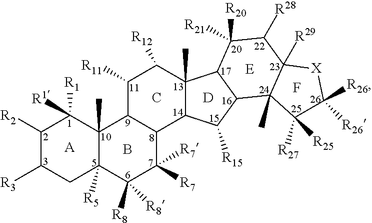
wherein each occurrence of - - - - - - is a single or double covalent bond;
wherein X is selected from O, NR^{x} and C(R^{x})₂;
wherein each occurrence of R^{x}, when present, is independently selected from hydrogen and C1-C6 alkyl;
wherein R¹ is selected from hydrogen, halogen, —OH, —CN, —NC, —NCO, —OCN, —NO₂, —ONO₂, —ONO, —NO, —N₃, —NH₂, —NH₃, —N═NR³¹, —NHOH, C1-C12 alkyl, C2-C12 alkenyl, C2-C12 alkynyl, C1-C12 hydroxy, C1-C12 alkoxy, C1-C12 thioalkyl, C1-C12 alkylthiol, C1-C12 aminoalkyl, C1-C12 alkylamino, (C1-C12)(C1-C12) dialkylamino, —OC(O)(C1-C12 alkyl), —OP(O)(OR³²)₂, —OSO₂R³³, —C(O)(C1-C12 alkyl), —CO₂R³⁴, —C(O)NR^{35a}R^{35b}, —(C1-C12 alkyl)C(O)NR^{35a}R^{35b}, —OC(O)NR^{35a}R^{35b}, —(C1-C12 alkyl)OC(O)NR^{35a}R^{35b}, Cy¹, Ar¹, (C1-C12 alkyl)Ar¹, and —OAr¹;
wherein each occurrence of R³¹, R³², R³⁴, R^{35a}, and R^{35b}, when present, is independently selected from hydrogen and C1-C12 alkyl;
wherein each occurrence of R³³, when present, is independently selected from hydrogen, C1-C12 alkyl, and monocyclic aryl monosubstituted with a methyl group;
wherein each occurrence of Cy¹, when present, is heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino;
wherein each occurrence of Ar¹, when present, is selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; and
wherein R^{1′} is hydrogen;
or wherein each of R¹ and R^{1′} together comprise ═O or ═NR³⁶;
wherein each occurrence of R³⁶, when present, is independently selected from hydrogen and C1-C12 alkyl;
wherein each of R² and R³ is independently selected from hydrogen, —OH, C1-C12 hydroxy, and halogen;
or wherein each of R₂ and R₃ together comprise —O—;
wherein R⁵ is selected from hydrogen, —OH, —NH₂, C1-C9 alkyl, C1-C9 hydroxy, C1-C9 alkoxy, C1-C9 aminoalkyl, C1-C6 alkylamino, and (C1-C6)(C1-C6) dialkylamino;
or wherein R⁵ is absent;
or wherein each of R⁷ and R^{7′} together comprise ═O;
or wherein one of R⁷ and R^{7′} is absent;
wherein each of R⁸ and R^{8′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr¹, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, —NR⁴¹C(O)(C1-C30 alkyl)-L-Z, —NR⁴¹C(O)(C1-C30 alkyl)-L-(C1-C30 alkyl)-Z, and —NR⁴¹C(O)-L-(C1-C30 alkyl)-Z;
wherein L is a linker;
wherein Z is selected from an antibody, an antibody fragment, a vitamin, a hormone, a carbohydrate, a molecular ligand, an aptamer, a non-antibody protein, a peptide, a nucleic acid, a fluorophore, and a drug;
wherein each occurrence of R⁴¹ and R⁴², when present, is independently selected from hydrogen and C1-C12 alkyl;
wherein each occurrence of Ar², when present, is independently selected from aryl and heteroaryl, and is substituted with 0, 1, 2, or 3 groups independently selected from halogen, —OH, —NH2, C1-C4 alkoxy, C1-C4 hydroxy, C1-C4 aminoalkyl, C1-C4 alkylamino, (C1-C4)(C1-C4) dialkylamino, —(C1-C12 alkyl)Ar⁴, and Ar⁴;
wherein each occurrence of Ar⁴, when present, is a structure represented by a formula: 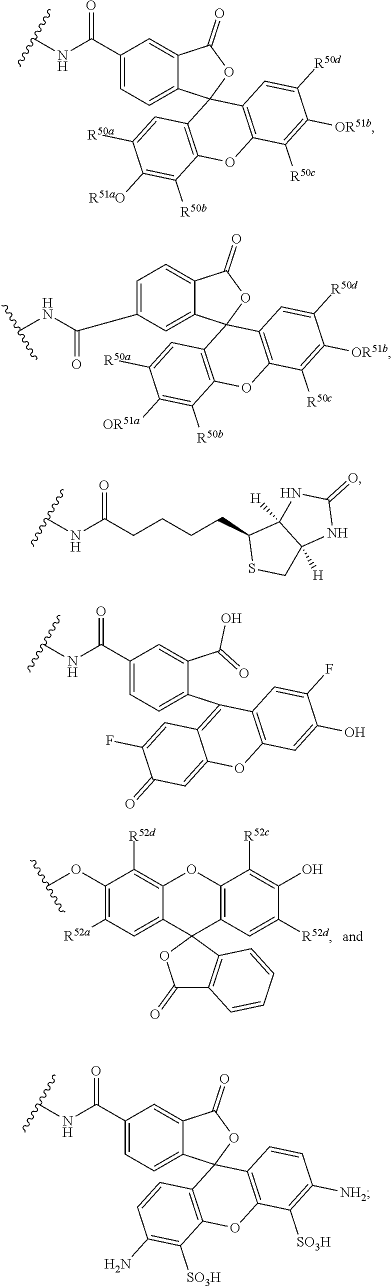
wherein each of R^{50a}, R^{50b}, R^{50c}, and R^{50d}, when present, is independently selected from hydrogen, —F, and —C;
wherein each of R^{51a} and R^{51b}, when present, is independently selected from hydrogen and —C(O)(C1-C12 alkyl);
wherein each of R^{52a}, R^{52b}, R^{52c}, and R^{52d}, when present, is independently selected from hydrogen, —F, and —Cl;
wherein each occurrence of Ar³, when present, is a structure represented by a formula selected from: 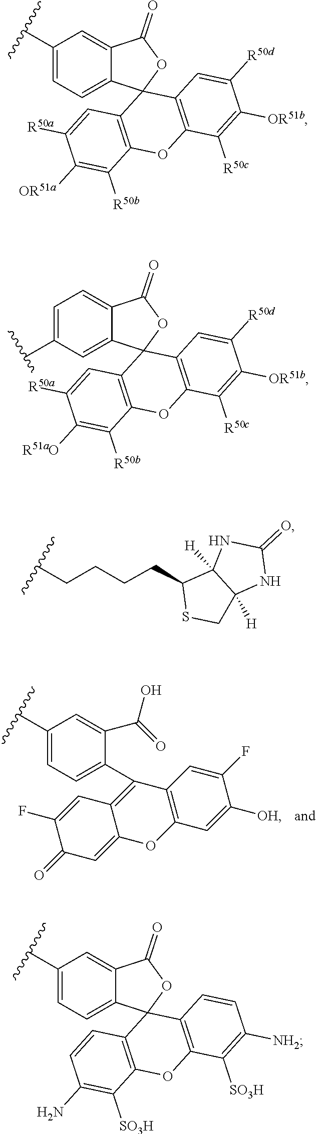
or wherein one of R⁸ and R^{8′} is absent;
wherein R⁷ is selected from hydrogen, —OH, C1-C30 hydroxy, C1-C30 alkoxy, —OC(O)(C1-C30 alkyl), and —OC(O)NR^{35a}R^{35b}, and wherein R^{7′} is selected from hydrogen, —OH, C1-C30 hydroxy, C1-C30 alkoxy, and —OC(O)(C1-C30 alkyl);
wherein each of R¹¹ and R¹² is independently selected from hydrogen, —OH, C1-C8 hydroxy, C1-C6 alkyl, C1-C8 alkoxy, and —OC(O)(C1-C8 alkyl);
wherein R¹⁵ is selected from hydrogen, —OH, C1-C30 hydroxy, C1-C30 alkyl, C1-C30 alkoxy, —OC(O)(C1-C30 alkyl), —OC(O)NR^{35a}R^{35b}, —OC(O)Ar², —OC(O)(C1-C4 alkyl)Ar², and —OC(O)(C1-C8 azide);
wherein R²⁰ is selected from hydrogen, —OH, —OOH, C1-C8 alkyl, C1-C8 hydroxy, C1-C8 alkoxy, C1-C8 hydroperoxy, and —OC(O)(C1-C8 alkyl);
wherein R²¹ is selected from hydrogen and C1-C6 alkyl;
wherein R²⁵ is selected from hydrogen, —OH, C1-C8 hydroxy, C1-C8 alkoxy, —OC(O)(C1-18 alkyl), —OC(O)NR^{35a}R^{35b}, —OC(O)Ar⁵, and —OC(O)(C1-C8 azide);
wherein Ar⁵, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino;
wherein each of R²⁶ and R^{26′} is independently selected from hydrogen, —OH, C1-C8 hydroxy, and C1-C8 alkoxy;
or wherein each of R²⁶ and R^{26′} together comprise ═O;
wherein R²⁷ is selected from hydrogen and C1-C6 alkyl;
wherein each of R²⁸ and R²⁹ is independently selected from hydrogen and halogen;
or wherein each of R²⁸ and R²⁹ together comprise —O— or —N(R³⁷)—;
wherein R³⁷, when present, is selected from hydrogen, C1-C4 alkyl, —SO₂R⁷¹, and a structure having a formula: 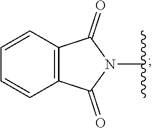 [SMILES: *N1C(=O)c2ccccc2C1=O]
and
wherein R⁷¹, when present, is selected from hydrogen, C1-C4 alkyl, —CH₂CH₂Si(CH₃)₃, and monocyclic aryl monosubstituted with a methyl group,
provided that one and only one of R⁸ and R^{8′} is —OC(O)(C1-C30 alkyl)-L-Z, —OC(O)(C1-C30 alkyl)-L-(C1-C30 alkyl)-Z, —OC(O)-L-(C1-C30 alkyl)-Z, —NR⁴¹C(O)(C1-C30 alkyl)-L-Z, —NR⁴¹C(O)(C1-C30 alkyl)-L-(C1-C30 alkyl)-Z, or —NR⁴¹C(O)-L-(C1-C30 alkyl)-Z, and
provided that when one or both of R²⁸ and R²⁹ is hydrogen then the occurrence of - - - - - - at C-22/C-23 is a double covalent bond,
or a pharmaceutically acceptable salt thereof, the method comprising reacting a taccalonolide compound having a structure represented by a formula: 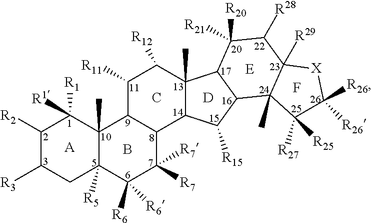
wherein each of R⁶ and R^{6′} is independently selected from hydrogen, halogen, —OH, —CN, —NC, —NCO, —OCN, —NO₂, —ONO₂, —ONO, —NO, —N₃, —NH₂, —NH₃, —N═NR⁴¹, —NHOH, C1-C30 alkyl, C2-C30 alkenyl, C2-C30 alkynyl, C1-C30 hydroxy, C1-C30 alkoxy, C1-C30 thioalkyl, C1-C30 alkylthiol, C1-C30 aminoalkyl, C1-C30 alkylamino, (C1-C30)(C1-C30) dialkylamino, —C(O)(C1-C30 alkyl), —OP(O)(OR³²)₂, —OSO₂R³³, —CO₂R³⁴, —C(O)NR^{35a}R^{35b}, —(C1-C30 alkyl)C(O)NR^{35a}R^{35b}, —OC(O)NR^{35a}R^{35b}, —(C1-C30 alkyl)OC(O)NR^{35a}R^{35b}, Cy¹, Ar¹, —(C1-C30 alkyl)Ar¹, —OAr1, —OC(O)(C1-C30 alkyl), —OC(O)Ar², —OC(O)(C1-C30 alkyl)Ar², —OC(O)Ar³, —OC(O)(C1-C30 alkyl)NR⁴²C(O)Ar³, —OC(O)(C1-C30 alkyl)OC(O)Ar³, —OC(O)R⁴⁰, —NR⁴¹C(O)(C1-C30 alkyl), —NR⁴¹C(O)Ar², —NR⁴¹C(O)(C1-C30 alkyl)Ar², —NR⁴¹C(O)Ar³, —NR⁴¹C(O)(C1-C30 alkyl)OC(O)Ar³, —NR⁴¹C(O)(C1-C30 alkyl)NR⁴²C(O)Ar³, and —NR⁴¹C(O)R⁴⁰; and
wherein R⁴⁰ is a C1-C30 alkyl functionalized with a group selected from —N₃, —SH, —OH, —NH₂, —NHOH, an ester, a disulfide, a sulfonamide, a terminal alkyne, haloacetyl, maleimide, isothiocyanate, N-hydroxysuccinimde, an acylhydrazine, an acylhydrazone, a hydrazine, a hydrazone, and hydrazide,
provided that one and only one of R⁶ and R^{1′} is —OC(O)R⁴⁰ or —NR⁴¹C(O)R⁴⁰,
with a nucleophile having a structure represented by a formula selected from:
H-L-(C1-C30 alkyl)-Z, H—(C1-C30 alkyl)-L-Z, and H—(C1-C30 alkyl)-L-(C1-C30 alkyl)-Z.

30. The method of claim 29, wherein R⁴⁰ is an ester selected from a succinimidyl ester, a tetrafluorophenyl ester, and a sulfodichlorophenol ester.


SEQUENCE LISTING (20 sequences)

SEQ ID NO: 1 - protein, 34 aa, Artificial sequence
  synthetic construct; primer
CAACCAGATCGGTGCCGCGTTCTGGGAGGTGATC

SEQ ID NO: 2 - protein, 34 aa, Artificial sequence
  synthetic construct; primer
GATCACCTCCCAGAACGCGGCACCGATCTGGTTG

SEQ ID NO: 3 - protein, 38 aa, Artificial sequence
  synthetic construct; primer
GATATCTGCTTCCGCACTGCGAAGCTGACCACACCAAC

SEQ ID NO: 4 - protein, 38 aa, Artificial sequence
  synthetic construct; primer
GTTGGTGTGGTCAGCTTCGCAGTGCGGAAGCAGATATC

SEQ ID NO: 5 - protein, 34 aa, Artificial sequence
  synthetic construct; primer
CTTCCGCACTCTGAAGGCGACCACACCAACCTAC

SEQ ID NO: 6 - protein, 34 aa, Artificial sequence
  synthetic construct; primer
GTAGGTTGGTGTGGTCGCCTTCAGAGTGCGGAAG

SEQ ID NO: 7 - protein, 31 aa, Artificial sequence
  synthetic construct; primer
CACCAACCTACGGGGCTCTGAACCACCTTGT

SEQ ID NO: 8 - protein, 31 aa, Artificial sequence
  synthetic construct; primer
ACAAGGTGGTTCAGAGCCCCGTAGGTTGGTG

SEQ ID NO: 9 - protein, 33 aa, Artificial sequence
  synthetic construct; primer
CACACCAACCTACGGGAATCTGAACCACCTTGT

SEQ ID NO: 10 - protein, 33 aa, Artificial sequence
  synthetic construct; primer
ACAAGGTGGTTCAGATTCCCGTAGGTTGGTGTG

SEQ ID NO: 11 - protein, 30 aa, Artificial sequence
  synthetic construct; primer
GAAGCTGACCACACCAGCCTACGGGGATCT

SEQ ID NO: 12 - protein, 30 aa, Artificial sequence
  synthetic construct; primer
AGATCCCCGTAGGCTGGTGTGGTCAGCTTC

SEQ ID NO: 13 - protein, 34 aa, Artificial sequence
  synthetic construct; primer
CTACGGGGATCTGAACGCCCTTGTCTCAGCCACC

SEQ ID NO: 14 - protein, 34 aa, Artificial sequence
  synthetic construct; primer
GGTGGCTGAGACAAGGGCGTTCAGATCCCCGTAG

SEQ ID NO: 15 - protein, 28 aa, Artificial sequence
  synthetic construct; primer
CCCTCTCACCAGCGCTGGAAGCCAGCAG

SEQ ID NO: 16 - protein, 28 aa, Artificial sequence
  synthetic construct; primer
CTGCTGGCTTCCAGCGCTGGTGAGAGGG

SEQ ID NO: 17 - protein, 22 aa, Artificial sequence
  synthetic construct; human Pgp primer
AAAGCGACTGAATGTTCAGTGG

SEQ ID NO: 18 - protein, 22 aa, Artificial sequence
  synthetic construct; Human Pgp primer
AATAGATGCCTTTCTGTGCCAG

SEQ ID NO: 19 - protein, 19 aa, Artificial sequence
  synthetic construct; Human GAPDH primer
GCAAATTCCATGGCACCGT

SEQ ID NO: 20 - protein, 19 aa, Artificial sequence
  synthetic construct; Human GAPDH primer
TCGCCCCACTTGATTTTGG